(19) United States
(12) Patent Application Publication
(10) Pub. No.: US 2023/0077723 A1
(43) Pub. Date: Mar. 16, 2023

(54) N-SUBSTITUTED-3,4-(FUSED 5-RING)-5-PHENYL-PYRROLIDINE-2-ONE COMPOUNDS AS INHIBITORS OF ISOQC AND/OR QC ENZYME

(71) Applicant: Scenic Immunology B.V., Amsterdam (NL)

(72) Inventors: Bastiaan Evers, Amsterdam (NL); Paul E. Brennan, Oxford (GB)

(73) Assignee: Scenic Immunology B.V., Amsterdam (NL)

(21) Appl. No.: 17/626,614

(22) PCT Filed: Jul. 10, 2020
(86) PCT No.: PCT/EP2020/069630

§ 371 Date: Jan. 12, 2022

(30) Foreign Application Priority Data
Jul. 12, 2019 (EP) 19185997.4

(51) Int. Cl. C07D 487/04; C07D 513/04; C07D 519/00

(52) U.S. Cl. CPC C07D 487/04; C07D 519/00; C07D 513/04

(57) ABSTRACT

The present invention pertains generally to the field of therapeutic compounds. More specifically the present invention pertains to certain N-substituted-3,4-(fused 5-ring)-5-phenyl-pyrrolidin-2-one compounds (also referred to herein as “FRPPO compounds”), that, inter alia, inhibit glutaminyl-peptide cyclotransferase-like (isoQC) enzyme and/or glutaminyl-peptide cyclotransferase (QC) enzyme (e.g., inhibit or reduce or block the activity or function of isoQC and/or QC enzyme). The present invention also pertains to pharmaceutical compositions comprising such compounds, and the use of such compounds and compositions, both in vitro and in vivo, to inhibit isoQC and/or QC enzyme; to treat disorders that are ameliorated by the inhibition of isoQC and/or QC enzyme; to treat cancer, atherosclerosis, fibrotic diseases, infectious diseases, Alzheimer's disease, etc.

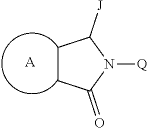

### TECHNICAL FIELD

The present invention pertains generally to the field of therapeutic compounds. More specifically the present invention pertains to certain N-substituted-3,4-(fused 5-ring)-5-phenyl-pyrrolidin-2-one compounds (also referred to herein as “FRPPO compounds”), that, inter alia, inhibit glutaminyl-peptide cyclotransferase-like (isoQC) enzyme and/or glutaminyl-peptide cyclotransferase (QC) enzyme (e.g., inhibit or reduce or block the activity or function of isoQC and/or QC enzyme). The present invention also pertains to pharmaceutical compositions comprising such compounds, and the use of such compounds and compositions, both in vitro and in vivo, to inhibit isoQC and/or QC enzyme; to treat disorders that are ameliorated by the inhibition of isoQC and/or QC enzyme; to treat cancer, atherosclerosis, fibrotic diseases, infectious diseases, Alzheimer's disease, etc.

### BACKGROUND

Publications are cited herein in order to more fully describe the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

This disclosure includes information that may be useful in understanding the present invention. It is not an admission that any of the information provided herein is prior art or relevant to the presently claimed invention, or that any publication specifically or implicitly referenced is prior art.

### Cancer

Cancer is a leading cause of death worldwide. Several therapies to treat and/or cure cancer have been developed over the years including, e.g., chemotherapy, radiation, surgery and cancer immunotherapy.

Cancer immunotherapy represents a type of cancer treatment designed to boost the body's natural immune defences to fight cancer. Overall, the purpose of cancer immunotherapy is to promote the ability of the immune system, including the innate immune system, to specifically detect and destroy cancer cells (e.g., via phagocytosis) while leaving healthy cells unaffected.

However, cancer cells are able to evade immune surveillance in many ways, for instance, by evading phagocytosis by e.g., macrophages or neutrophils through the expression of so-called “anti-phagocytic” or “don't eat me” signals. One prominent example of such signal is the interaction between the “Cluster of Differentiation 47” and “signal-regulatory protein alpha” proteins.

### The CD47-SIRPα Signalling Axis

The term “Cluster of Differentiation 47” (abbreviated as “CD47”) as used herein refers to a 50 kDa transmembrane protein (receptor) encoded by the CD47 gene (Ensembl reference: ENSG00000196776 in human). CD47 is also known as integrin associated protein (IAP). CD47 is expressed by many cells in the body as revealed by CD47 mRNA expression and CD47 immunohistochemical staining studies (see, e.g., Wiersma et al., 2015; Lindberg et al., 1993). CD47 has been implicated in a range of cellular processes, including apoptosis, proliferation, adhesion, and migration as well as angiogenic and immune responses. CD47 binds to or interacts with several ligands with signal-regulatory protein alpha (SIRPα) being considered a main ligand for CD47.

The term “signal-regulatory protein alpha” (abbreviated “SIRPα” or “SIRPa”, also termed CD172a or SHPS-1) as used herein refers to a regulatory transmembrane glycoprotein from the SIRP family, which is encoded by the SIRPα gene (Ensembl reference: ENSG00000198053 in human). SIRPα is an inhibitory transmembrane receptor of various cell types, including myeloid cells (e.g., macrophages, monocytes, neutrophils, basophils, eosinophils, dendritic cells), neurons, and (in vitro) cardiomyocytes derived from induced pluripotent stem cells (see, e.g., Matozaki et al., 2009; Dubois et al., 2011).

The interaction between CD47 and SIRPα mediates or conveys “anti-phagocytic” or “don't eat me” signals between two cells, which ultimately inhibit phagocytosis and other cytotoxic effects. When CD47 interacts or binds with SIRPα, it initiates a cascade of signaling events in the cells (i.e., the cell expressing CD47 and the cell expressing SIRPα). Specifically, CD47-SIRPα interaction causes tyrosine phosphorylation of SIRPα cytoplasmic immunoreceptor tyrosine-based inhibitory motifs (ITIM) motifs, which in turn leads to concomitant recruitment of Src homology 2 domain tyrosine phosphatase 1 (SHP-1) and Src homology 2 domain tyrosine phosphatase 2 (SHP-2). SHP-1 and SHP-2 are cytoplasmic protein tyrosine phosphatases, which mediate signaling events causing inhibition of phagocytosis by for instance dephosphorylating myosin-IIA (see, e.g., Wiersma et al., 2015).

Because it triggers a cascade of signaling events leading to inhibition of phagocytosis, the binding or interacting of CD47 with SIRPα is often referred to as a “don't eat me signal” or “anti-phagocytic signal”. Importantly, the binding of CD47 to SIRPα can also inhibit death of CD47 expressing cells by other mechanisms, such as antibody dependent cellular cytotoxicity (ADCC). Together, blocking of the CD47-SIRPα interaction between target cells and immune cells may be exploited to enhance death of the CD47 expressing cells.

It has been reported that cancer cells upregulate the expression of CD47 at their cell surface, which results in CD47 levels which are higher compared to CD47 levels found in normal cells (see, e.g., Majeti et al., 2009; Chao et al., 2012). As a result, cancer cells can evade destruction by the immune system or evade immune surveillance, e.g., by evading phagocytosis by immune cells such as phagocyte cells (e.g., macrophages, neutrophils). It was also found that diseased cells in conditions other than cancer, such as, e.g., atherosclerosis, fibrotic diseases, infectious diseases caused by pathogens (e.g., viruses), etc., upregulate the expression of CD47 at their cell surface compared to the CD47 levels found in normal/healthy cells to evade phagocytosis by phagocytes. Hence, inhibition of the CD47-SIRPα interaction may also be therapeutically exploited in these and yet other, to be discovered, disease areas. However, also in conditions where CD47 is not specifically upregulated, blocking of the CD47-SIRPα interaction may be therapeutically beneficial by shifting the balance towards phagocytosis of target cells.

In the case of cancer, several approaches to interfere with the CD47-SIRPα interaction have principally targeted CD47. For instance, several anti-CD47 antibodies and recombinant SIRPα proteins, either or not fused to immunomodulatory peptide sequences, aimed at interfering or blocking CD47-SIRPα interactions are currently being developed or tested in clinical trials. Although promising, such strategies have important drawbacks. For instance, antibodies and large polypeptides are known to have poor tissue penetration, especially into solid tumors, as compared to small molecule inhibitors. Furthermore, since CD47 is widely distributed throughout the body, including healthy tissues, the available pool of molecules able to bind to the intended cells is limited. This is referred to as the antigen sink effect. Other disadvantages associated with the use of anti-CD47 antibodies include the lack of oral bioavailability and undesirable side effects such as the development of anemia (which may occur as a result of a dose-dependent loss of red blood cells) as well as hemagglutination (clumping of red blood cells) and thrombocytopenia (lack of blood platelets). Alternative therapeutic strategies that do not rely on large biomolecules (e.g., antibodies or recombinant proteins) could yield improved efficacy, less toxic side-effects, and increased ease of use.

Pyroglutamylation of the N-Terminal Glutamine Moiety of CD47: isoQC and QC

Recently, it has been demonstrated that binding of SIRPα to CD47 depends on pyroglutamylation of the N-terminal glutamine moiety of CD47. Pyroglutamylation is a post-translational modification in which either a glutamine or glutamate amino acid is converted into a pyroglutamate moiety. The human genome contains two genes that encode two enzymes that catalyse this N-terminal pyroglutamylation reaction, i.e., the glutaminyl-peptide cyclotransferase-like (QPCTL) gene encoding the glutaminyl-peptide cyclotransferase-like (isoQC) protein/enzyme and the glutaminyl-peptide cyclotransferase (QPCT) gene encoding the glutaminyl-peptide cyclotransferase (QC) protein/enzyme. While isoQC is localized in the Golgi apparatus and QC is secreted, there is an overlap in substrate preference and enzymatic characteristics.

In case of the interaction between SIRPα and CD47, this interaction was shown to primarily depend on the glutaminyl cyclase activity of the isoQC protein, encoded by the glutaminyl-peptide cyclotransferase-like (QPCTL) gene. As such, it was shown that chemical inhibition of isoQC or genetic depletion of QPCTL reduced SIRPα binding to CD47. Furthermore, increased phagocytosis of antibody opsonized cells in vitro and increased clearance of opsonized tumour cells in vivo were shown to be effects of blocking isoQC activity or production (see, e.g., Logtenberg et al., 2019). Due to the overlap in enzymatic characteristics and substrate preference, it is possible that, in certain circumstances, glutaminyl cyclase activity of the QC protein can also play a role in the interaction between SIRPα and CD47 and therefore in the CD47-SIRPα signalling axis.

Accordingly, reducing or blocking or inhibiting the activity of the enzyme referred to as glutaminyl-peptide cyclotransferase-like (isoQC) and/or the enzyme referred to as glutaminyl-peptide cyclotransferase (QC) is associated with a reduction or inhibition or blockade of the interaction or binding between CD47 and SIRPα. This reduction of interaction or binding between CD47 and SIRPα results in a reduction or inhibition or blockade of the CD47-SIRPα signaling axis.

Generation of a proficient “anti-phagocytic signal” or “do not eat me signal” by a cell (for instance a cell in a disease or condition involving the CD47-SIRPα signaling axis, such as a cancer cell) could therefore be blocked by interfering with the enzyme(s) responsible for the pyroglutamylation of CD47, including isoQC and possibly under certain conditions QC.

In addition to CD47, QC and isoQC pyroglutamylate other proteins. For example, the amyloid beta protein involved in Alzheimer's disease is known to be pyroglutamylated by QC and the C—C Motif Chemokine Ligand 2 (CCL2) protein is known to be pyroglutamylated by isoQC. Due to the large overlap in enzymatic characteristics and substrate preference it can, however, not be ruled out that there is a certain amount of functional overlap between QC and isoQC in pyroglutamylating these targets.

The inventors have identified a class of small molecule compounds that inhibit the glutaminyl-peptide cyclotransferase-like (isoQC) enzyme and/or the glutaminyl-peptide cyclotransferase (QC) enzyme (e.g., inhibit or reduce or block the activity or function of isoQC and/or QC enzyme), and may offer, for example, improved potency and/or improved selectivity as compared to known inhibitors.

### Some Known Compounds

Each of Blank et al., 2014a, Blank et al., 2014b, Blank et al., 2014c, and Blank et al., 2014d describes certain compounds of the following formula as bromodomain and extra-terminal motif (BET) inhibitors for use in the treatment of cancer.

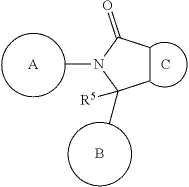

Blank et al., 2015 describes certain compounds of the following formula as bromodomain and extra-terminal motif (BET) inhibitors for use in the treatment of cancer.

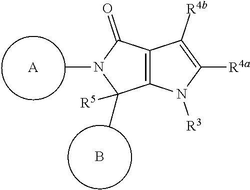

### SUMMARY OF THE INVENTION

One aspect of the invention pertains to certain N-substituted-3,4-(fused 5-ring)-5-phenyl-pyrrolidin-2-one compounds (referred to herein as FRPPO compounds), as described herein.

Another aspect of the invention pertains to a composition (e.g., a pharmaceutical composition) comprising a FRPPO compound, as described herein, and a pharmaceutically acceptable carrier or diluent.

Another aspect of the invention pertains to a method of preparing a composition (e.g., a pharmaceutical composition) comprising the step of mixing a FRPPO compound, as described herein, and a pharmaceutically acceptable carrier or diluent.

Another aspect of the present invention pertains to a method of inhibiting glutaminyl-peptide cyclotransferase-like (isoQC) enzyme and/or glutaminyl-peptide cyclotransferase (QC) enzyme (e.g., inhibiting or reducing or blocking the activity or function of isoQC and/or QC enzyme), in vitro or in vivo, comprising contacting the isoQC and/or QC enzyme with an effective amount of a FRPPO compound, as described herein.

Another aspect of the present invention pertains to a method of inhibiting glutaminyl-peptide cyclotransferase-like (isoQC) enzyme and/or glutaminyl-peptide cyclotransferase (QC) enzyme (e.g., inhibiting or reducing or blocking the activity or function of isoQC and/or QC enzyme) in a cell, in vitro or in vivo, comprising contacting the cell with an effective amount of a FRPPO compound, as described herein.

Another aspect of the present invention pertains to a FRPPO compound as described herein for use in a method of treatment of the human or animal body by therapy, for example, for use in a method of treatment of a disorder (e.g., a disease) as described herein.

Another aspect of the present invention pertains to use of a FRPPO compound as described herein in a method of treatment of the human or animal body by therapy, for example, in a method of treatment of a disorder (e.g., a disease) as described herein.

Another aspect of the present invention pertains to use of a FRPPO compound, as described herein, in the manufacture of a medicament, for example, for use in a method of treatment, for example, for use in a method of treatment of a disorder (e.g., a disease) as described herein.

Another aspect of the present invention pertains to a method of treatment, for example, a method of treatment of a disorder (e.g., a disease) as described herein, comprising administering to a subject in need of treatment a therapeutically-effective amount of a FRPPO compound, as described herein, preferably in the form of a pharmaceutical composition.

In one embodiment, the disorder is a disorder that is ameliorated by the inhibition of glutaminyl-peptide cyclotransferase-like (isoQC) enzyme and/or glutaminyl-peptide cyclotransferase (QC) enzyme (e.g., by the inhibition or reduction or blockage of the activity or function of isoQC and/or QC enzyme).

In one embodiment, the disorder is, for example, cancer, atherosclerosis, a fibrotic disease, an infectious disease, Alzheimer's disease, etc., as described herein.

Another aspect of the present invention pertains to a kit comprising (a) a FRPPO compound, as described herein, preferably provided as a composition (e.g., a pharmaceutical composition) and in a suitable container and/or with suitable packaging; and (b) instructions for use, for example, in a method of treatment of a disorder (e.g., a disease) as described herein, for example, written instructions on how to administer the compound.

Another aspect of the present invention pertains to a FRPPO compound obtainable by a method of synthesis as described herein, or a method comprising a method of synthesis as described herein.

Another aspect of the present invention pertains to a FRPPO compound obtained by a method of synthesis as described herein, or a method comprising a method of synthesis as described herein.

Another aspect of the present invention pertains to novel intermediates, as described herein, which are suitable for use in the methods of synthesis described herein.

Another aspect of the present invention pertains to the use of such novel intermediates, as described herein, in the methods of synthesis described herein.

As will be appreciated by one of skill in the art, features and preferred embodiments of one aspect of the invention will also pertain to other aspects of the invention.

### DETAILED DESCRIPTION OF THE INVENTION

### Compounds

One aspect of the present invention relates to compounds of the following general formula, wherein Ring A, -J and -Q are as defined herein (for convenience, collectively referred to herein as “N-substituted-3,4-(fused 5-ring)-5-phenyl-pyrrolidin-2-one compounds” or “FRPPO compounds”):

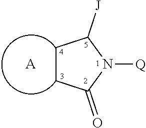

Some embodiments of the compounds include the following:

(1) A compound of the following formula, or a pharmaceutically acceptable salt, hydrate, or solvate thereof:

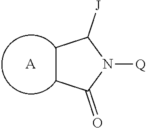

wherein Ring A is a 5-membered heteroaromatic ring having:
exactly 1 ring heteroatom, wherein the ring heteroatom is N; or
exactly 2 ring heteroatoms, wherein each ring heteroatom is N; or
exactly 2 ring heteroatoms, wherein one ring heteroatom is N and the other ring heteroatom is O; or
exactly 2 ring heteroatoms, wherein one ring heteroatom is N and the other ring heteroatom is S; or
exactly 3 ring heteroatoms, wherein each ring heteroatom is N;
   and wherein, in Ring A:
a non-bridging ring atom that is N may optionally be substituted with a group —R^{ANN};
a non-bridging ring atom that is C may optionally be substituted with a group —R^{ACC};
   wherein —R^{ACC} or each —R^{ACC} if there are two or more, is independently selected from:
—R^{T},
—R^{TX},
F, —Cl, —Br, —I,
—OH, —OR^{TT}, —OR^{TX},
L^{TT}-OH, -L^{TT}-OR^{TT}, -L^{TT}-OR^{TX},
—NH₂, —NHR^{TT}, —NR^{TT}₂, —NHR^{TX},
L^{TT}-NH₂, -L^{TT}-NHR^{TT}, -L^{TT}-NR^{TT}₂,
—C(═O)R^{TT},
—C(═O)OH, —C(═O)OR^{TT}, —OC(═O)R^{TT},
—C(═O)NH₂, —C(═O)NHR^{TT}, —C(═O)NR^{TT}₂,
—NHC(═O)R^{TT}, —NR^{TN}C(═O)R^{TT}
—NHC(═O)NH₂, —NHC(═O)NHR^{TT}, —NHC(═O)NR^{TT}₂,
—NR^{TN}C(═O)NH₂, —NR^{TN}C(═O)NHR^{TT}, —NR^{TN}C(═O)NR^{TT}₂,
—NHC(═O)OR^{TT}, —NR^{TN}C(═O)OR^{TT},
—OC(═O)NH₂, —OC(═O)NHR^{TT}, —OC(═O)NR^{TT},
—S(═O)₂NH₂, —S(═O)₂NHR^{TT}, —S(═O)₂NR^{TT}₂,
—NHS(═O)₂R^{TT}, —NR^{TN}S(═O)₂R^{TT},
—S(═O)(═NH)—NH₂, —S(═O)(═NH)—NHR^{TT}, —S(═O)(═NH)—NR^{TT}₂,
—S(═O)(═NR^{TT})—NH₂, —S(═O)(═NR^{TT})—NHR^{TT}, —S(═O)(═NR^{TT})—NR^{TT}₂,
—N═S(═O)(R^{TT})—NH₂, —N═S(═O)(R^{TT})—NHR^{TT}, —N═S(═O)(R^{TT})—NR^{TT}₂,
—NH—S(═O)(═NH)—R^{TT}, —NH—S(═O)(═NR_{TT})—R^{TT},
—NR^{TN}—S(═O)(═NH)—R^{TT}, —NR^{TN}—S(═O)(═NR^{TT})—R^{TT},
—S(═O)R^{TT}, —S(═O)₂R^{TT},
—SH, —SR^{TT}, —SR^{TX},
—CN, and —NO₂;
   wherein —R^{ANN}, or each —R^{ANN} if there are two or more, is independently selected from:
—R^{T},
—R^{TX},
L^{TT}-OH, -L^{TT}-OR^{TT}, -L^{TT}-OR^{TX},
L^{TT}-NH₂, -L^{TT}-NHR^{TT}, -L^{TT}-NR^{TT}₂,
—C(═O)R^{TT},
—C(═O)OR^{TT},
—C(═O)NH₂, —C(═O)NHR^{TT}, —C(═O)NR^{TT}₂,
—S(═O)₂NH₂, —S(═O)₂NHR^{TT}, —S(═O)₂NR^{TT}₂,
—S(═O)R^{TT}, and —S(═O)₂R^{TT};
   wherein:
each —R^{T} is independently selected from:
   —R^{T1}, —R^{T2}, —R^{T3}, —R^{T4}, —R^{T5},
   LT-R^{T2}, -LT-R^{T3}, -LT-R^{T4}, and -LT-R^{T5};
each —R^{TT} is independently selected from:
   —R^{T1}, —R^{T2}, —R^{T3}, —R^{T4}, —R^{T5},
   LT-R^{T2}, -LT-R^{T3}, -LT-R^{T4}, and -LT-R^{T5};
each —R^{TX} is independently linear or branched saturated C₁₋₄fluoroalkyl;
each —R^{TN} is independently linear or branched saturated C₁₋₄alkyl;
each -L^{TT}- is independently linear or branched saturated C₁₋₄alkylene;
   wherein:
each —R^{T1} is independently linear or branched saturated C₁₋₆alkyl;
each —R^{T2} is saturated C₃₋₆cycloalkyl;
each —R^{T3} is non-aromatic C₄₋₉heterocyclyl;
each —R^{T4} is independently phenyl or naphthyl;
each —R^{T5} is C₅₋₁₂heteroaryl;
each -L^{T}- is independently linear or branched saturated C₁₋₄alkylene;
   wherein each —R^{T2}, —R^{T3}, —R^{T4}, and —R^{T5} is optionally substituted with one or more groups independently selected from:
—R^{TTT}, —R^{TTTX},
—F, —Cl, —Br, —I,
—OH, —OR^{TTT}, —OR^{TTTX},
—NH₂, —NHR^{TTT}, —NHR^{TTTX}, —NR^{TTT}₂,
—C(═O)R^{TTT}, —C(═O)OH, and —C(═O)OR^{TTT};
   wherein:
each —R^{TTT} is independently selected from linear or branched saturated C₁₋₄alkyl, saturated C₃₋₆cycloalkyl, phenyl, and benzyl;
each —R^{TTTX} is independently linear or branched saturated C₁₋₄fluoroalkyl;
   and wherein -Q is independently selected from:

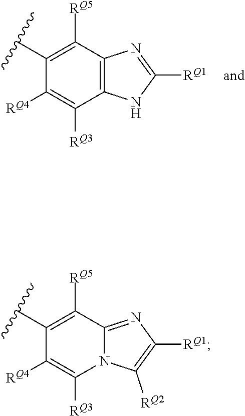

wherein:
each —R^{Q1} is independently —H or —R^{QQ1};
—R^{Q2} is independently —H or —R^{QQ2};
each —R^{Q3} is independently —H or —R^{QQ3};
each —R^{Q4} is independently —H or —R^{QQ4};
each —R_{Q5} is independently —H or —R_{QQ5}; and
each —R^{QQ1}, —R^{QQ2}, —R^{QQ3} —R^{QQ4}, and —R^{QQ5} is independently —R^{Q};
   wherein each —R^{Q} is independently selected from:
—R^{QQ},
—R^{QX},
—F, —Cl, —Br, —I,
—OH, —OR^{QQ}, —OR^{QX},
—NH₂, —NHR^{QQ}, —NHR^{QX}, —NR^{QQ2}, and
—CN;
   wherein:
each —R^{QQ} is independently —R^{QQQ1} or —R^{QQQ2};
each —R^{QQQ1} is independently linear or branched saturated C₁₋₄alkyl;
each —R^{QQQ2} is saturated C₃₋₆cycloalkyl;
each —R^{QX} is independently linear or branched saturated C₁₋₄fluoroalkyl;
   and wherein -J is the following group:

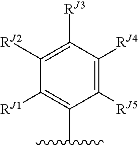

wherein:
—R^{J1} is independently —H or —R^{JJ1};
—R^{J2} is independently —H or —R^{JJ2};
—R^{J3} is independently —H or —R_{JJ3};
—R^{J4} is independently —H or —R_{JJ4}; and
—R^{J5} is independently —H or —R^{JJ5};
   wherein:
each of —R^{JJ1}, —R^{JJ2}, —R^{JJ3} —R^{JJ4} and —R^{JJ5} is independently —R^{J};
   wherein each —R^{J} is independently selected from:
—R^{P},
—R^{PX},
F, —Cl, —Br, —I,
—OH, —OR^{PP}, —OR^{PX},
L^{PP}-OH, -L^{PP}-OR^{PP}, -L^{PP}-OR^{PX},
—NH₂, —NHR^{PP}, —NR^{PP}₂, —NHR^{PX},
L^{PP}-NH₂, -L^{PP}-NHR^{PP}, -L^{PP}-NR^{PP}₂,
—C(═O)R^{PP},
—C(═O)OH, —C(═O)OR^{PP}, —OC(═O)R^{PP},
—C(═O)NH₂, —C(═O)NHR^{PP}, —C(═O)NR^{PP}₂,
—NHC(═O)R^{PP}, —NR^{PN}C(═O)R^{PP},
—NHC(═O)NH₂, —NHC(═O)NHR^{PP}, —NHC(═O)NR^{PP}₂,
—NR^{PN}C(═O)NH₂, —NR^{PN}C(═O)NHR^{PP}, —NR^{PN}C(═O)NR^{PP}₂,
—NHC(═O)OR^{PP}, —NR^{PN}C(═O)OR^{PP},
—OC(═O)NH₂, —OC(═O)NHR^{PP}, —OC(═O)NR^{PP}₂,
—S(═O)₂NH₂, —S(═O)₂NHR^{PP}, —S(═O)₂NR^{PP}₂,
—NHS(═O)₂R^{PP}, —NR^{PN}S(═O)₂R^{PP},
—S(═O)(═NH)—NH₂, —S(═O)(═NH)—NHR^{PP}, —S(═O)(═NH)—NR^{PP}₂,
—S(═O)(═NR^{PP})—NH₂, —S(═O)(═NR^{PP})—NHR^{PP}, —S(═O)(═NR^{PP})—NR^{PP}₂,
—N═S(═O)(R^{PP})—NH₂, —N═S(═O)(R^{PP})—NHR^{PP}, —N═S(═O)(R^{PP})—NR^{PP}₂,
—NH—S(═O)(═NH)—R^{PP}, —NH—S(═O)(═NR^{PP})—R^{PP},
—NR^{PN}—S(═O)(═NH)—R^{PP}, —NR^{PN}—S(═O)(═NR^{PP})—R^{PP},
—S(═O)R^{PP}, —S(═O)₂R^{PP},
—SH, —SR^{PP}, —SR^{PX},
—CN, and —NO₂;
   wherein:
each —R^{P} is independently selected from:
   —R^{P1}, —R^{P2}, —R^{P3}, —R^{P4}, —R^{P5},
   LP-R^{P2}, -LP-R^{P3}, -LP-R^{P4}, and -LP-R^{P5};
each —R^{PP} is independently selected from:
   —R^{P1}, —R^{P2}, —R^{P3}, —R^{P4}, —R^{P5},
   LP-R^{P2}, -LP-R^{P3}, -LP-R^{P4}, and -LP-R^{P5};
each —R^{P}X is independently linear or branched saturated C₁₋₄fluoroalkyl;
each —R^{PN} is independently linear or branched saturated C₁₋₄-alkyl;
each -L^{PP}- is independently linear or branched saturated Cu-alkylene;
   wherein:
each —R^{P1} is independently linear or branched saturated C₁₋₆alkyl;
each —R^{P2} is saturated C₃₋₆cycloalkyl;
each —R^{P3} is non-aromatic C₄₋₉heterocyclyl;
each —R^{P4} is independently phenyl or naphthyl;
each —R^{P5} is C₅₋₁₂heteroaryl;
each -LP- is independently linear or branched saturated C₁₋₄alkylene;
   wherein each —R^{P2}, —R^{P3}, —R^{P4}, and —R^{P5} is optionally substituted with one or more groups independently selected from:
—R^{PPP}, —R^{PPPX},
—F, —Cl, —Br, —I,
—OH, —OR^{PPP}, —OR^{PPPX},
—NH₂, —NHR^{PPP}, —NHR^{PPPX}, —NR^{PPP}₂,
—C(═O)R^{PPP}, —C(═O)OH, and —C(═O)OR^{PPP},
—S(═O)₂R^{PPP}; and
—CN;
   and wherein, additionally, each —R^{P2} and —R^{P3} is optionally substituted with ═O (e.g., one or two ═O);
   wherein:
each —R^{PPP} is independently selected from linear or branched saturated C₁₋₄alkyl, saturated C₃₋₆cycloalkyl, phenyl, and benzyl;
each —R^{PPPX} is independently linear or branched saturated C₁₋₄fluoroalkyl;
   and additionally:

—R^{JJ1} and —R^{JJ2}, if present, taken together with the atoms to which they are attached, may form a fused 5- or 6-membered ring (i.e., fused to the phenyl ring to which they are attached); or

—R^{JJ2} and —R^{JJ3}, if present, taken together with the atoms to which they are attached, may form a fused 5- or 6-membered ring (i.e., fused to the phenyl ring to which they are attached).

For the avoidance of doubt, it is not intended that the groups -Q and -J are linked other than via the ring atoms to which they are attached. For example, it is not intended that -Q and -J together form a fused ring structure.

For the avoidance of doubt, it is not intended that Ring A and the group -J are linked other than via the ring atoms to which they are attached. For example, it is not intended that Ring A and -J together form a fused ring structure.

For the avoidance of doubt, it is not intended that Ring A and the group -Q are linked other than via the ring atoms to which they are attached. For example, it is not intended that Ring A and -Q together form a fused ring structure.

For the avoidance of doubt, when Q is benzimidazole, or substituted benzimidazole, it is not intended that the N ring atom at the 1-position is substituted; instead, it is intended that the N ring atom at the 1-position is unsubstituted.

Unless otherwise indicated, where a compound is shown or described which has one or more chiral centres, and two or more stereoisomers are possible, all such stereoisomers are disclosed and encompassed, both individually (e.g., as isolated from the other stereoisomer(s)) and as mixtures (e.g., as equimolar or non-equimolar mixtures of two or more stereoisomers). For example, unless otherwise indicated, where a compound has one chiral centre, each of the (R) and (S) enantiomers are disclosed and encompassed, both individually (e.g., as isolated from the other enantiomer) and as a mixture (e.g., as equimolar or non-equimolar mixtures of the two enantiomers).

Note that the compounds have at least one chiral centre, specifically, the ring carbon atom to which -J is attached, marked with an asterisk (*) in the following formula. Unless otherwise stated, the carbon atom at this position may be in either (R) or (S) configuration.

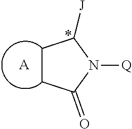

Note that, depending upon the identity of Ring A, and any substituents thereon, Ring A may be susceptible to tautomerism. For example, compounds of the following formulae are tautomers of each other:

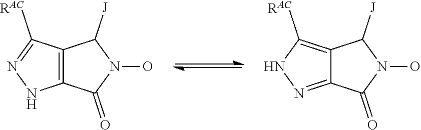

Unless otherwise indicated, where a compound is shown or described which is susceptible to tautomerism, and two tautomers are possible, both tautomers are disclosed and encompassed, both individually (e.g., as isolated from the other tautomer) and as mixtures (e.g., as equimolar or non-equimolar mixtures of two tautomer).

The phrase “on carbon”, used in respect of substituents, —R^{Q}, on the group -Q means that each —R^{Q}, if present, is attached to a non-bridging aromatic ring carbon atom of the group -Q.

The term “saturated linear or branched C₁₋₃alkyl” means —CH₃ (methyl), —CH₂CH₃ (ethyl), —CH₂CH₂CH₃ (n-propyl), and —CH(CH₃)₂ (iso-propyl).

The term “saturated linear or branched C₁₋₄alkyl” additionally includes —CH₂CH₂CH₂CH₃ (n-butyl), —CH₂CH(CH₃)₂ (iso-butyl), —CH(CH₃)CH₂CH₃ (sec-butyl), and —C(CH₃)₃ (tert-butyl).

The term “saturated linear or branched C₁₋₆alkyl” additionally includes, e.g., —CH₂CH₂CH₂CH₂CH₃ (n-pentyl), —CH₂CH₂CH(CH₃)₂ (iso-pentyl), —CH₂CH₂CH₂CH₂CH₂CH₃ (n-hexyl), —CH₂CH₂CH₂CH(CH₃)₂ (iso-hexyl), etc.

The term “saturated linear or branched C₁₋₄fluoroalkyl” means a saturated linear or branched C₁₋₄alkyl group substituted with one or more fluoro groups, and includes, e.g., —CF₃, —CHF₂, —CH₂CF₃, —CH₂CH₂F, —CH₂CHF₂, —CH(CH₃)CF₃, —CH₂C(CH₃)₂F, —CH₂CF₂CH₃, —CH₂CH₂CF₂CH₃, —CH₂CH₂CHF₂, —CH₂CH₂CF₃, etc.

The term “saturated C₃₋₆cycloalkyl” means cyclopropyl, cyclobutyl, cyclopentyl, and cyclohexyl.

The term “linear or branched saturated C₁₋₄alkylene” means a bi-dentate saturated linear or branched C₁₋₄alkyl group, and includes, e.g., —CH₂—, —CH₂CH₂—, —CH₂CH₂CH₂—, —CH₂CH₂CH₂CH₂—, —CH(CH₃)—, —CH₂CH(CH₃)—, etc.

The term “non-aromatic C₄₋₉heterocyclyl” means a non-aromatic cyclic group having 5 to 7 ring atoms, wherein exactly 1, exactly 2, or exactly 3 of the ring atoms is a ring heteroatom, wherein each ring heteroatom is selected from O, N, and S (wherein a ring S atom may optionally be in an oxidized form, e.g., S(═O) or S(═O)₂). Such groups may be monocyclic or polycyclic, e.g., bridged or spiro. Examples include, e.g., non-aromatic monocyclic C₇heterocyclyl, such as oxetanyl, tetrahydrofuranyl, tetra hydropyranyl, oxanyl, dioxanyl, azetidinyl, pyrrolidinyl, piperidinyl, piperazinyl, morpholinyl, thiomorpholinyl, 1,4-thiazinane 1,1-dioxide, azepanyl, oxazepanyl, and diazepanyl; non-aromatic bridged C₇₋₉heterocyclyl, such as those derived from the compounds shown below; and non-aromatic spiro C₇₋₉heterocyclyl, such as those derived from the compounds shown below.

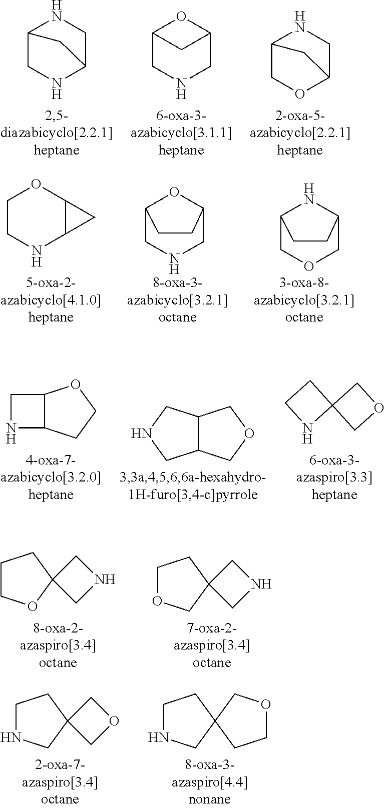

The term “C₅₋₁₂heteroaryl” means an aromatic group having 5 to 12 ring atoms, wherein exactly 1, exactly 2, or exactly 3 of the aromatic ring atoms is a ring heteroatom, wherein each ring heteroatom is selected from O, N, and S. Such groups may be monocyclic or polycyclic, e.g., fused. Examples include, e.g., C₅₋₆heteroaryl groups, such as furanyl, thienyl, pyrrolyl, imidazolyl, oxazolyl, isoxazolyl, thiazolyl, isothiazolyl, pyrazolyl, triazolyl, oxadiazolyl, thiadiazolyl, pyridyl, pyridazinyl, pyrimidinyl, and pyrazinyl; and C₉₋₁₂heteroaryl groups, such as indolyl, benzimidazolyl, indazolyl, benzofuranyl, benzothienyl, benzoxazolyl, benzothiazolyl, benzisoxazolyl, benzisothiazolyl, quinolinyl, isoquinolinyl, cinnolinyl, quinoxalinyl, quinazolinyl, and phthalazinyl.

### Ring A: N₁

(2) A compound according to (1), wherein Ring A is a 5-membered heteroaromatic ring having exactly 1 ring heteroatom, wherein the ring heteroatom is N.
(3) A compound according to (1), which is compound of one of the following formulae, or a pharmaceutically acceptable salt, hydrate, or solvate thereof:

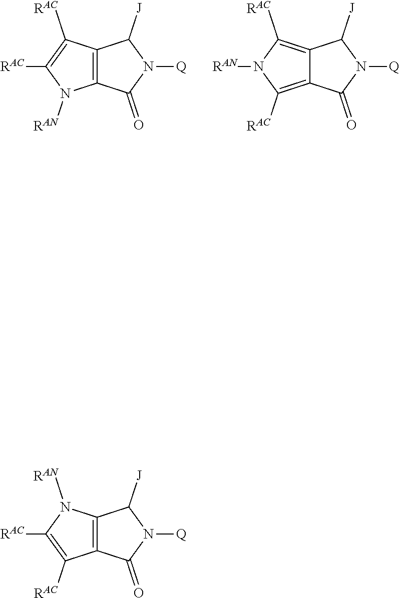

wherein:
each —R^{AC} is independently —H or —R^{ACC}; and
each —R^{AN} is independently —H or —R^{ANN}.
   (4) A compound according to (1), which is compound of the following formula, or a pharmaceutically acceptable salt, hydrate, or solvate thereof:

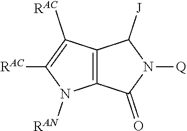

wherein:
each —R^{AC} is independently —H or —R^{ACC}; and
—R^{AN} is independently —H or —R^{ANN}.
   (5) A compound according to (1), which is compound of the following formula, or a pharmaceutically acceptable salt, hydrate, or solvate thereof:

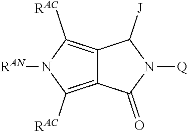

wherein:
each —R^{AC} is independently —H or —R^{ACC}; and
—R^{AN} is independently —H or —R^{ANN}.
   (6) A compound according to (1), which is compound of the following formula, or a pharmaceutically acceptable salt, hydrate, or solvate thereof:

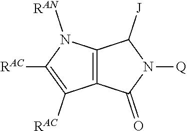

wherein:
each —R^{AC} is independently —H or —R^{ACC}; and
—R^{AN} is independently —H or —R^{ANN}.

### Ring A: N₂

(7) A compound according to (1), wherein Ring A is a 5-membered heteroaromatic ring having exactly 2 ring heteroatoms, wherein each ring heteroatom is N.
(8) A compound according to (1), which is compound of one of the following formulae, or a pharmaceutically acceptable salt, hydrate, or solvate thereof:

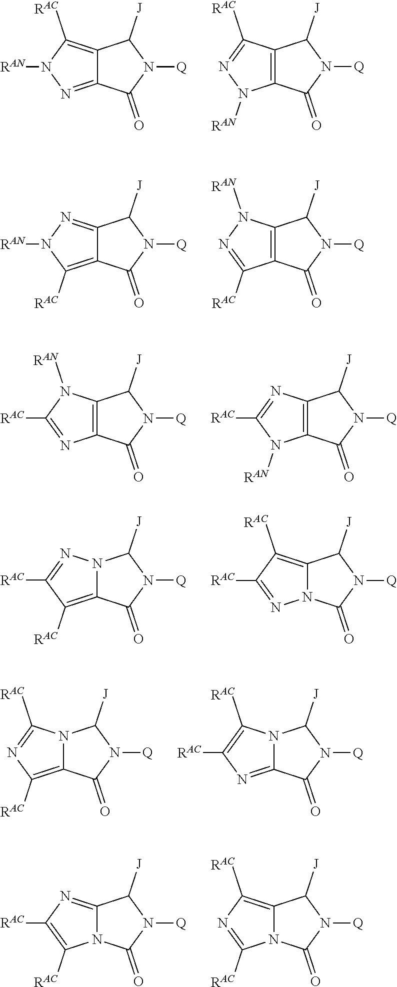

wherein:
each —R^{AC} is independently —H or —R^{ACC}; and
each —R^{AN} is independently —H or —R^{ANN}.
   (9) A compound according to (1), which is compound of one of the following formulae, or a pharmaceutically acceptable salt, hydrate, or solvate thereof:

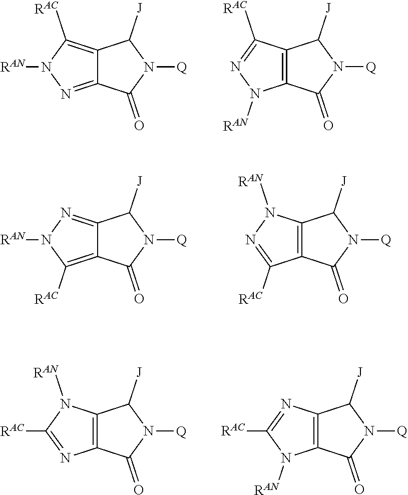

wherein:
each —R^{AC} is independently —H or —R^{ACC}; and
each —R^{AN} is independently —H or —R^{ANN}.
   (10) A compound according to (1), which is compound of one of the following formulae, or a pharmaceutically acceptable salt, hydrate, or solvate thereof:

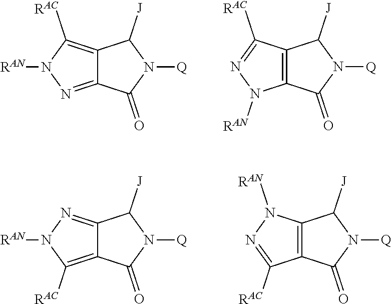

wherein:
each —R^{AC} is independently —H or —R^{ACC}; and
each —R^{AN} is independently —H or —R^{ANN}.
   (11) A compound according to (1), which is compound of one of the following formulae, or a pharmaceutically acceptable salt, hydrate, or solvate thereof:

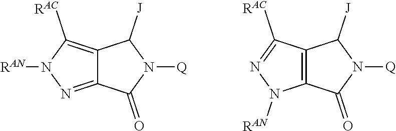

wherein:
each —R^{AC} is independently —H or —R^{ACC}; and
each —R^{AN} is independently —H or —R^{ANN}.
   (12) A compound according to (1), which is compound of the following formula, or a pharmaceutically acceptable salt, hydrate, or solvate thereof:

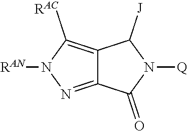

wherein:
—R^{AC} is independently —H or —R^{ACC}; and
—R^{AN} is independently —H or —R^{ANN}.
   (13) A compound according to (1), which is compound of the following formula, or a pharmaceutically acceptable salt, hydrate, or solvate thereof:

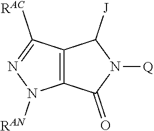

wherein:
—R^{AC} is independently —H or —R^{ACC}; and
—R^{AN} is independently —H or —R^{ANN}.
   (14) A compound according to (1), which is compound of one of the following formulae, or a pharmaceutically acceptable salt, hydrate, or solvate thereof:

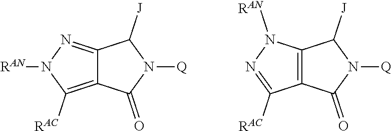

wherein:
each —R^{AC} is independently —H or —R^{ACC}; and
each —R^{AN} is independently —H or —R^{ANN}.
   (15) A compound according to (1), which is compound of the following formula, or a pharmaceutically acceptable salt, hydrate, or solvate thereof:

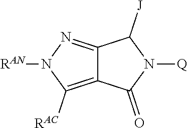

wherein:
—R^{AC} is independently —H or —R^{ACC}; and
—R^{AN} is independently —H or —R^{ANN}.
   (16) A compound according to (1), which is compound of the following formula, or a pharmaceutically acceptable salt, hydrate, or solvate thereof:

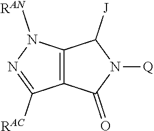

wherein:
—R^{AC} is independently —H or —R^{ACC}; and
—R^{AN} is independently —H or —R^{ANN}.
   (17) A compound according to (1), which is compound of one of the following formulae, or a pharmaceutically acceptable salt, hydrate, or solvate thereof:

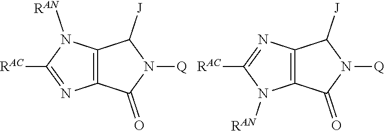

wherein:
each —R^{AC} is independently —H or —R^{ACC}; and
each —R^{AN} is independently —H or —R^{ANN}.
   (18) A compound according to (1), which is compound of the following formula, or a pharmaceutically acceptable salt, hydrate, or solvate thereof:

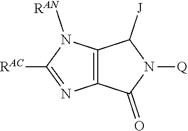

wherein:
—R^{AC} is independently —H or —R^{ACC}; and
—R^{AN} is independently —H or —R^{ANN}.
   (19) A compound according to (1), which is compound of the following formula, or a pharmaceutically acceptable salt, hydrate, or solvate thereof:

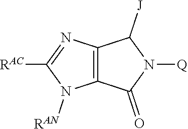

wherein:
—R^{AC} is independently —H or —R^{ACC}; and
—R^{AN} is independently —H or —R^{ANN}.
   (20) A compound according to (1), which is compound of one of the following formulae, or a pharmaceutically acceptable salt, hydrate, or solvate thereof:

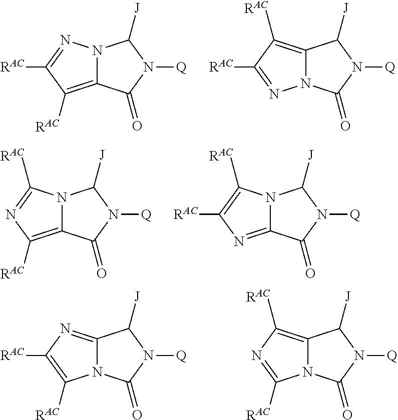

wherein:
each —R^{AC} is independently —H or —R^{ACC}.
   (21) A compound according to (1), which is compound of one of the following formulae, or a pharmaceutically acceptable salt, hydrate, or solvate thereof:

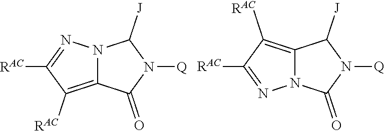

wherein:
each —R^{AC} is independently —H or —R^{ACC}.
   (22) A compound according to (1), which is compound of the following formula, or a pharmaceutically acceptable salt, hydrate, or solvate thereof:

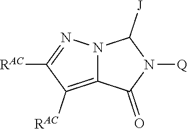

wherein:
each —R^{AC} is independently —H or —R^{ACC}.
   (23) A compound according to (1), which is compound of the following formula, or a pharmaceutically acceptable salt, hydrate, or solvate thereof:

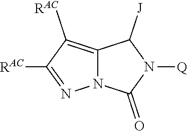

wherein:
each —R^{AC} is independently —H or —R^{ACC}.
   (24) A compound according to (1), which is compound of one of the following formulae, or a pharmaceutically acceptable salt, hydrate, or solvate thereof:

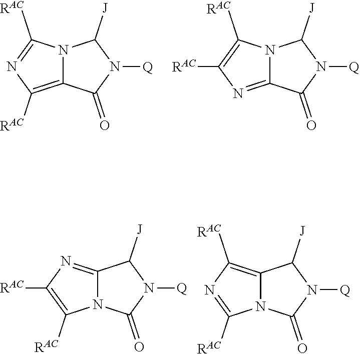

wherein:
each —R^{AC} is independently —H or —R^{ACC}.
   (25) A compound according to (1), which is compound of one of the following formulae, or a pharmaceutically acceptable salt, hydrate, or solvate thereof:

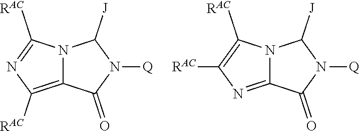

wherein:
each —R^{AC} is independently —H or —R^{ACC}.
   (26) A compound according to (1), which is compound of the following formula, or a pharmaceutically acceptable salt, hydrate, or solvate thereof:

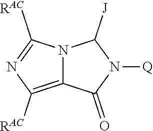

wherein:
each —R^{AC} is independently —H or —R^{ACC}.
   (27) A compound according to (1), which is compound of the following formula, or a pharmaceutically acceptable salt, hydrate, or solvate thereof:

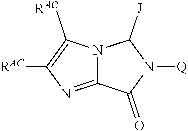

wherein:
each —R^{AC} is independently —H or —R^{ACC}.
   (28) A compound according to (1), which is compound of one of the following formulae, or a pharmaceutically acceptable salt, hydrate, or solvate thereof:

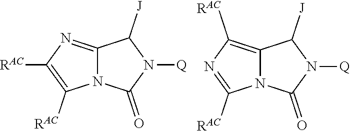

wherein:
each —R^{AC} is independently —H or —R^{ACC}.
   (29) A compound according to (1), which is compound of the following formula, or a pharmaceutically acceptable salt, hydrate, or solvate thereof:

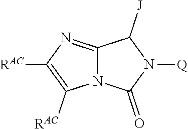

wherein:
each —R^{AC} is independently —H or —R^{ACC}.
   (30) A compound according to (1), which is compound of the following formula, or a pharmaceutically acceptable salt, hydrate, or solvate thereof:

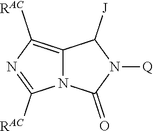

wherein:
each —R^{AC} is independently —H or —R^{ACC}.

### Ring A: NO

(31) A compound according to (1), wherein Ring A is a 5-membered heteroaromatic ring having exactly 2 ring heteroatoms, wherein one ring heteroatom is N and the other ring heteroatom is O.
(32) A compound according to (1), which is compound of one of the following formulae, or a pharmaceutically acceptable salt, hydrate, or solvate thereof:

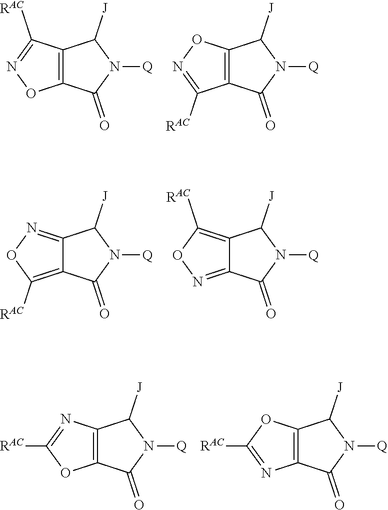

wherein:
each —R^{AC} is independently —H or —R^{ACC}.
   (33) A compound according to (1), which is compound of one of the following formulae, or a pharmaceutically acceptable salt, hydrate, or solvate thereof:

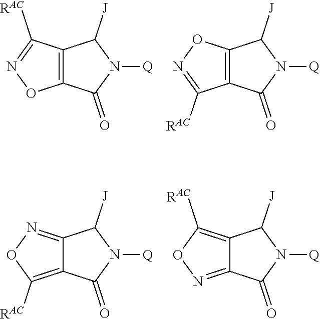

wherein:
each —R^{AC} is independently —H or —R^{ACC}.
   (34) A compound according to (1), which is compound of one of the following formulae, or a pharmaceutically acceptable salt, hydrate, or solvate thereof:

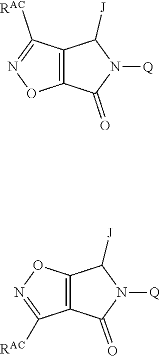

wherein:
each —R^{AC} is independently —H or —R^{ACC}.
   (35) A compound according to (1), which is compound of the following formula, or a pharmaceutically acceptable salt, hydrate, or solvate thereof:

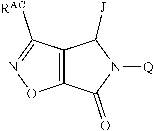

wherein:
—R^{AC} is independently —H or —R^{ACC}.
   (36) A compound according to (1), which is compound of the following formula, or a pharmaceutically acceptable salt, hydrate, or solvate thereof:

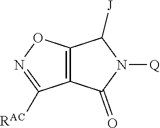

wherein:
—R^{AC} is independently —H or —R^{ACC}.
   (37) A compound according to (1), which is compound of one of the following formulae, or a pharmaceutically acceptable salt, hydrate, or solvate thereof:

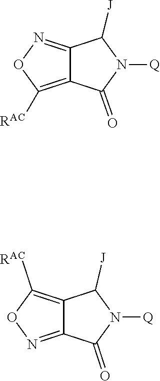

wherein:
each —R^{AC} is independently —H or —R^{ACC}.
   (38) A compound according to (1), which is compound of the following formula, or a pharmaceutically acceptable salt, hydrate, or solvate thereof:

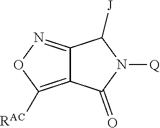

wherein:
—R^{AC} is independently —H or —R^{ACC}.
   (39) A compound according to (1), which is compound of the following formula, or a pharmaceutically acceptable salt, hydrate, or solvate thereof:

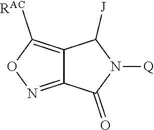

wherein:
—R^{AC} is independently —H or —R^{ACC}.
   (40) A compound according to (1), which is compound of one of the following formulae, or a pharmaceutically acceptable salt, hydrate, or solvate thereof:

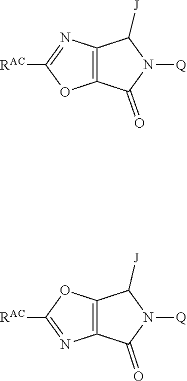

wherein:
each —R^{AC} is independently —H or —R^{ACC}.
   (41) A compound according to (1), which is compound of the following formula, or a pharmaceutically acceptable salt, hydrate, or solvate thereof:

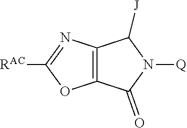

wherein:
—R^{AC} is independently —H or —R^{ACC}.
   (42) A compound according to (1), which is compound of the following formula, or a pharmaceutically acceptable salt, hydrate, or solvate thereof:

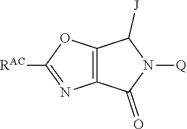

wherein:
—R^{AC} is independently —H or —R^{ACC}.

### Ring A: NS

(43) A compound according to (1), wherein Ring A is a 5-membered heteroaromatic ring having exactly 2 ring heteroatoms, wherein one ring heteroatom is N and the other ring heteroatom is S.
(44) A compound according to (1), which is compound of one of the following formulae, or a pharmaceutically acceptable salt, hydrate, or solvate thereof:

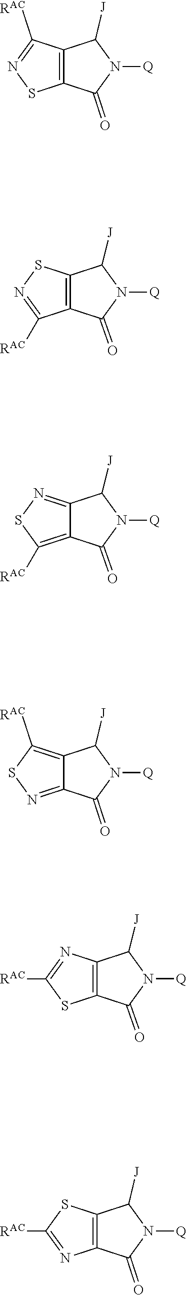

wherein:
each —R^{AC} is independently —H or —R^{ACC}.
   (45) A compound according to (1), which is compound of one of the following formulae, or a pharmaceutically acceptable salt, hydrate, or solvate thereof:

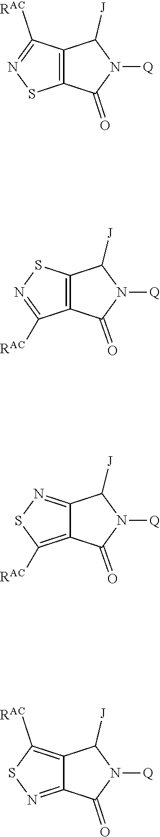

wherein:
each —R^{AC} is independently —H or —R^{ACC}.
   (46) A compound according to (1), which is compound of one of the following formulae, or a pharmaceutically acceptable salt, hydrate, or solvate thereof:

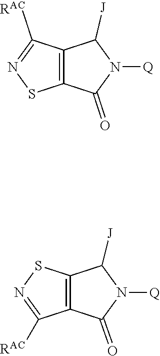

wherein:
each —R^{AC} is independently —H or —R^{ACC}.
   (47) A compound according to (1), which is compound of the following formula, or a pharmaceutically acceptable salt, hydrate, or solvate thereof:

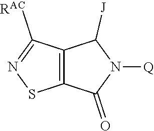

wherein:
—R^{AC} is independently —H or —R^{ACC}.
   (48) A compound according to (1), which is compound of the following formula, or a pharmaceutically acceptable salt, hydrate, or solvate thereof:

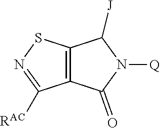

wherein:
—R^{AC} is independently —H or —R^{ACC}.
   (49) A compound according to (1), which is compound of one of the following formulae, or a pharmaceutically acceptable salt, hydrate, or solvate thereof:

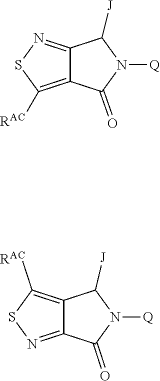

wherein:
each —R^{AC} is independently —H or —R^{ACC}.
   (50) A compound according to (1), which is compound of the following formula, or a pharmaceutically acceptable salt, hydrate, or solvate thereof:

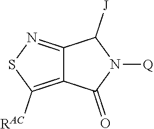

wherein:
—R^{AC} is independently —H or —R^{ACC}.
   (51) A compound according to (1), which is compound of the following formula, or a pharmaceutically acceptable salt, hydrate, or solvate thereof:

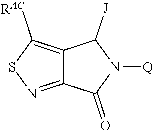

wherein:
—R^{AC} is independently —H or —R^{ACC}.
   (52) A compound according to (1), which is compound of one of the following formulae, or a pharmaceutically acceptable salt, hydrate, or solvate thereof:

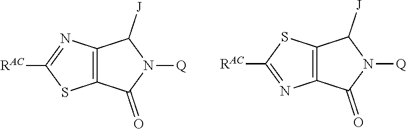

wherein:
each —R^{AC} is independently —H or —R^{ACC}.
   (53) A compound according to (1), which is compound of the following formula, or a pharmaceutically acceptable salt, hydrate, or solvate thereof:

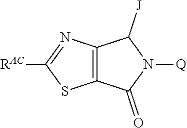

wherein:
—R^{AC} is independently —H or —R^{ACC}.
   (54) A compound according to (1), which is compound of the following formula, or a pharmaceutically acceptable salt, hydrate, or solvate thereof:

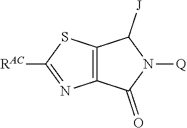

wherein:
—R^{AC} is independently —H or —R^{ACC}.

### Ring A: N₃

(55) A compound according to (1), wherein Ring A is a 5-membered heteroaromatic ring having exactly 3 ring heteroatoms, wherein each ring heteroatom is N.
(56) A compound according to (1), which is compound of one of the following formulae, or a pharmaceutically acceptable salt, hydrate, or solvate thereof:

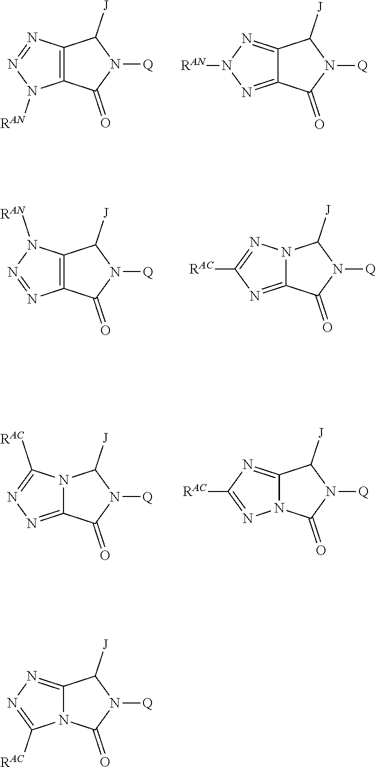

wherein:
each —R^{AC} is independently —H or —R^{ACC}; and
each —R^{AN} is independently —H or —R^{ANN}.
   (57) A compound according to (1), which is compound of one of the following formulae, or a pharmaceutically acceptable salt, hydrate, or solvate thereof:

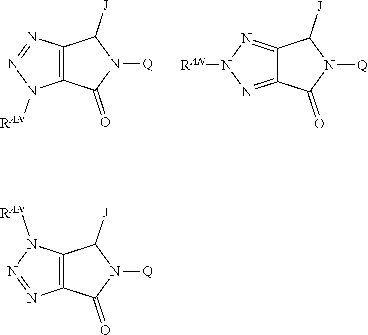

wherein:
each —R^{AN} is independently —H or —R^{ANN}.
   (58) A compound according to (1), which is compound of the following formula, or a pharmaceutically acceptable salt, hydrate, or solvate thereof:

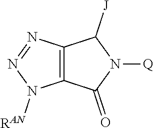

wherein:
—R^{AN} is independently —H or —R^{ANN}.
   (59) A compound according to (1), which is compound of the following formula, or a pharmaceutically acceptable salt, hydrate, or solvate thereof:

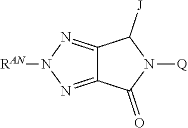

wherein:
—R^{AN} is independently —H or —R^{ANN}.
   (60) A compound according to (1), which is compound of the following formula, or a pharmaceutically acceptable salt, hydrate, or solvate thereof:

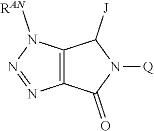

wherein:
—R^{AN} is independently —H or —R^{ANN}.
   (61) A compound according to (1), which is compound of one of the following formulae, or a pharmaceutically acceptable salt, hydrate, or solvate thereof:

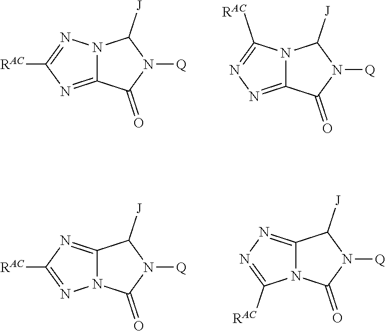

wherein:
each —R^{AC} is independently —H or —R^{ACC}; and
each —R^{AN} is independently —H or —R^{ANN}.
   (62) A compound according to (1), which is compound of one of the following formulae, or a pharmaceutically acceptable salt, hydrate, or solvate thereof:

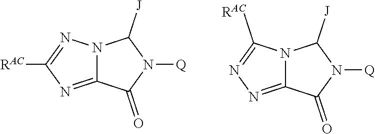

wherein:
each —R^{AC} is independently —H or —R^{ACC}.
   (63) A compound according to (1), which is compound of the following formula, or a pharmaceutically acceptable salt, hydrate, or solvate thereof:

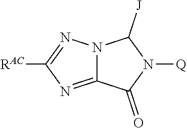

wherein:
—R^{AC} is independently —H or —R^{ACC}.
   (64) A compound according to (1), which is compound of the following formula, or a pharmaceutically acceptable salt, hydrate, or solvate thereof:

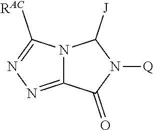

wherein:
—R^{AC} is independently —H or —R^{ACC}.
   (65) A compound according to (1), which is compound of one of the following formulae, or a pharmaceutically acceptable salt, hydrate, or solvate thereof:

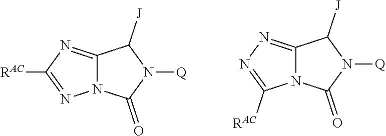

wherein:
each —R^{AC} is independently —H or —R^{ACC}.
   (66) A compound according to (1), which is compound of the following formula, or a pharmaceutically acceptable salt, hydrate, or solvate thereof:

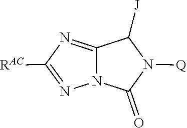

wherein:
—R^{AC} is independently —H or —R^{ACC}.
   (67) A compound according to (1), which is compound of the following formula, or a pharmaceutically acceptable salt, hydrate, or solvate thereof:

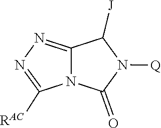

wherein:
—R^{AC} is independently —H or —R^{ACC}.

### The Substituents —R^{AC}

(68) A compound according to any one of (1) to (67), wherein —R^{AC}, if present, or each —R^{AC} if there are two or more, is H.
(69) A compound according to any one of (1) to (67), wherein —R^{AC}, if present, or each —R^{AC} if there are two or more, is —R^{ACC}.

### The Substituents —R^{AN}

(70) A compound according to any one of (1) to (69), wherein —R^{AN}, if present, or each —R^{AN} if there are two or more, is H.
(71) A compound according to any one of (1) to (69), wherein —R^{AN}, if present, or each —R^{AN} if there are two or more, is —R^{ANN}.

### The Substituents —R^{ACC}.

(72) A compound according to any one of (1) to (71), wherein —R^{ACC} if present, or each —R^{ACC} if there are two or more, is independently selected from:
—R^{T},
—R^{TX},
F, —Cl,
—OR^{TT}, —OR^{TX},
L^{TT}-OR^{TT}, -L^{TT}-OR^{TX},
—NHR^{TT}, —NR^{TT}₂,
L^{TT}-NR^{TT}₂,
—C(═O)R^{TT},
—S(═O)₂R^{TT},
—SR^{TT}, —SR^{TX}, and
—CN.
   (73) A compound according to any one of (1) to (71), wherein —R^{ACC}, if present, or each —R^{ACC} if there are two or more, is independently selected from:
—R^{T},
—R^{TX},
F, —Cl,
—OR^{TT}, —OR^{TX},
L^{TT}-OR^{TT}, -L^{TT}-OR^{TX},
—NHR^{TT}, —NR^{TT}₂,
—C(═O)R^{TT}, and
—CN.
   (74) A compound according to any one of (1) to (71), wherein —R^{ACC} if present, or each —R^{ACC} if there are two or more, is independently selected from:
—R^{T},
—R^{TX},
—F, —Cl,
—OR^{TT}, —OR^{TX},
—NHR^{TT}, —NR^{TT}₂, and
—CN.
   (75) A compound according to any one of (1) to (71), wherein —R^{ACC} if present, or each —R^{ACC} if there are two or more, is independently selected from: —R^{T} and —R^{TX}.
   (76) A compound according to any one of (1) to (71), wherein —R^{ACC}, if present, or each —R^{ACC} if there are two or more, is —R^{T}.

### The Substituents —R^{ANN}.

(77) A compound according to any one of (1) to (76), wherein —R^{ANN}, if present, or each —R^{ANN} if there are two or more, is independently selected from:
—R^{T},
—R^{TX},
L^{TT}-OH, -L^{TT}-OR^{TT}, -L^{TT}-OR^{TX},
L^{TT}-NR^{TT}₂,
—C(═O)R^{TT},
—C(═O)OR^{TT},
—C(═O)NHR^{TT}, —C(═O)NR^{TT}₂,
—S(═O)₂NHR^{TT}, —S(═O)₂NR^{TT}₂, and
—S(═O)₂R^{TT}.
   (78) A compound according to any one of (1) to (76), wherein —R^{ANN}, if present, or each —R^{ANN} if there are two or more, is independently selected from:
—R^{T},
—R^{TX},
L^{TT}-OR^{TT}, and -L^{TT}-OR^{TX}.
   (79) A compound according to any one of (1) to (76), wherein —R^{ANN}, if present, or each —R^{ANN} if there are two or more, is independently selected from: —R^{T} and —R^{TX}.
   (80) A compound according to any one of (1) to (76), wherein —R^{ANN}, if present, or each —R^{ANN} if there are two or more, is —R^{T}.

### The Group —R^{T}

(81) A compound according to any one of (1) to (80), wherein each —R^{T}, if present, is independently selected from:
—R^{T1}, —R^{T2}, —R^{T4}, -LT-R^{T2}, and -LT-R^{T4}.
   (82) A compound according to any one of (1) to (80), wherein each —R^{T}, if present, is independently selected from:
—R^{T1}, —R^{T2}, and -LT-R^{T2}.
   (83) A compound according to any one of (1) to (80), wherein each —R^{T}, if present, is —R^{T1}.

### The Group —R^{TT}

(84) A compound according to any one of (1) to (83), wherein each —R^{TT}, if present, is independently selected from:
—R^{T1}, —R^{T2}, —R^{T4}, -LT-R^{T2}, and -LT-R^{T4}.
   (85) A compound according to any one of (1) to (83), wherein each —R^{TT}, if present, is independently selected from:
—R^{T1}, —R^{T2}, and -L^{T}-R^{T2}.
   (86) A compound according to any one of (1) to (83), wherein each —R^{TT}, if present, is independently selected from: —R^{T1} and —R^{T2}.
   (87) A compound according to any one of (1) to (83), wherein each —R^{TT}, if present, is —R^{T1}.

### The Group —R^{TX}

(88) A compound according to any one of (1) to (87), wherein each —R^{TX}, if present, is independently selected from: —CF₃, —CHF₂, —CH₂CF₃, —CH₂CH₂F, —CH₂CHF₂, —CH(CH₃)CF₃, —CH₂C(CH₃)₂F, —CH₂CF₂CH₃, —CH₂CH₂CF₂CH₃, —CH₂CH₂CHF₂, and —CH₂CH₂CF₃.
(89) A compound according to any one of (1) to (87), wherein each —R^{TX}, if present, is independently selected from: —CF₃, —CHF₂, —CH₂CF₃, —CH₂CH₂F, —CH₂CHF₂, —CH(CH₃)CF₃, and —CH₂C(CH₃)₂F.
(90) A compound according to any one of (1) to (87), wherein each —R^{TX}, if present, is —CF₃.

### The Group —R^{TN}

(91) A compound according to any one of (1) to (90), wherein each —R^{TN}, if present, is independently linear or branched saturated C₁₋₃alkyl.
(92) A compound according to any one of (1) to (90), wherein each —R^{TN}, if present, is -Me.

### The Group -L^{TT}-

(93) A compound according to any one of (1) to (92), wherein each -L^{TT}-, if present, is independently selected from: —CH₂—, —CH₂CH₂—, —CH(CH₃)—, and —CH₂CH₂CH₂—.
(94) A compound according to any one of (1) to (92), wherein each -L^{TT}-, if present, is independently selected from: —CH₂— and —CH₂CH₂—.
(95) A compound according to any one of (1) to (92), wherein each -L^{TT}-, if present, is —CH₂—.

### The Group —R^{T1}

(96) A compound according to any one of (1) to (95), wherein each —R^{T1}, if present, is independently linear or branched saturated C₁₋₄alkyl.
(97) A compound according to any one of (1) to (95), wherein each —R^{T1}, if present, is independently linear or branched saturated C₁₋₃alkyl.
(98) A compound according to any one of (1) to (95), wherein each —R^{T1}, if present, is -Me.

### The Group —R^{T2}

(99) A compound according to any one of (1) to (98), wherein each —R^{T2}, if present, is independently selected from: cyclopropyl and cyclobutyl.

### The Group —R^{T3}

(100) A compound according to any one of (1) to (99), wherein each —R^{T3}, if present, is independently selected from: non-aromatic monocyclic C₄₋₇heterocyclyl; non-aromatic bridged C₇₋₉heterocyclyl; and non-aromatic spiro C₇₋₉heterocyclyl.
(101) A compound according to any one of (1) to (99), wherein each —R^{T3}, if present, is independently selected from: oxetanyl; tetrahydrofuranyl; tetrahydropyranyl; oxanyl; dioxanyl; azetidinyl; pyrrolidinyl; piperidinyl; piperazinyl; morpholinyl; thiomorpholinyl, 1,4-thiazinane 1,1-dioxide; azepanyl; oxazepanyl; diazepanyl; 2,5-diazabicyclo[2.2.1]heptane; 6-oxa-3-azabicyclo[3.1.1]heptane; 2-oxa-5-azabicyclo[2.2.1]heptane; 5-oxa-2-azabicyclo[4.1.0]heptane; 8-oxa-3-azabicyclo[3.2.1]octane; 3-oxa-8-azabicyclo[3.2.1]octane; 4-oxa-7-azabicyclo[3.2.0]heptane; 3,3a,4,5,6,6a-hexahydro-1H-furo[3,4-c]pyrrole; 6-oxa-3-azaspiro[3.3]heptane; 8-oxa-2-azaspiro[3.4]octane; 7-oxa-2-azaspiro[3.4]octane; 2-oxa-7-azaspiro[3.4]octane; and 8-oxa-3-azaspiro[4.4]nonane.
(102) A compound according to any one of (1) to (99), wherein each —R^{T3}, if present, is non-aromatic monocyclic C₄₋₇heterocyclyl.
(103) A compound according to any one of (1) to (99), wherein each —R^{T3}, if present, is independently selected from: oxetanyl, tetrahydrofuranyl, tetrahydropyranyl, oxanyl, dioxanyl, azetidinyl, pyrrolidinyl, piperidinyl, piperazinyl, morpholinyl, thiomorpholinyl, 1,4-thiazinane 1,1-dioxide, azepanyl, oxazepanyl, and diazepanyl.
(104) A compound according to any one of (1) to (99), wherein each —R^{T3}, if present, is independently selected from: azetidinyl, pyrrolidinyl, piperidinyl, piperazinyl, morpholinyl, thiomorpholinyl, azepanyl, and diazepanyl.
(105) A compound according to any one of (1) to (99), wherein each —R^{T3}, if present, is independently selected from: azetidino, pyrrolidino, piperidino, piperazino, morpholino, thiomorpholino, azepano, and diazepano.
(106) A compound according to any one of (1) to (99), wherein each —R^{T3}, if present, is independently selected from: non-aromatic bridged C₇₋₉heterocyclyl or non-aromatic spiro C₇₋₉heterocyclyl.
(107) A compound according to any one of (1) to (99), wherein each —R^{T3}, if present, is independently selected from: 2,5-diazabicyclo[2.2.1]heptane; 6-oxa-3-azabicyclo[3.1.1]heptane; 2-oxa-5-azabicyclo[2.2.1]heptane; 5-oxa-2-azabicyclo[4.1.0]heptane; 8-oxa-3-azabicyclo[3.2.1]octane; 3-oxa-8-azabicyclo[3.2.1]octane; 4-oxa-7-azabicyclo[3.2.0]heptane; 3,3a,4,5,6,6a-hexahydro-1H-furo[3,4-c]pyrrole; 6-oxa-3-azaspiro[3.3]heptane; 8-oxa-2-azaspiro[3.4]octane; 7-oxa-2-azaspiro[3.4]octane; 2-oxa-7-azaspiro[3.4]octane; and 8-oxa-3-azaspiro[4.4]nonane.
(108) A compound according to any one of (1) to (99), wherein each —R^{T3}, if present, is independently selected from N-linked: 2,5-diazabicyclo[2.2.1]heptane; 6-oxa-3-azabicyclo[3.1.1]heptane; 2-oxa-5-azabicyclo[2.2.1]heptane; 5-oxa-2-azabicyclo[4.1.0]heptane; 8-oxa-3-azabicyclo[3.2.1]octane; 3-oxa-8-azabicyclo[3.2.1]octane; 4-oxa-7-azabicyclo[3.2.0]heptane; 3,3a,4,5,6,6a-hexahydro-1H-furo[3,4-c]pyrrole; 6-oxa-3-azaspiro[3.3]heptane; 8-oxa-2-azaspiro[3.4]octane; 7-oxa-2-azaspiro[3.4]octane; 2-oxa-7-azaspiro[3.4]octane; and 8-oxa-3-azaspiro[4.4]nonane.

### The Group —R^{T4}

(109) A compound according to any one of (1) to (108), wherein each —R^{T4}, if present, is phenyl.

### The Group —R^{T5}

(110) A compound according to any one of (1) to (109), wherein each —R^{T5}, if present, is independently selected from: C₅₋₆heteroaryl or C₉₋₁₂heteroaryl.
(111) A compound according to any one of (1) to (109), wherein each —R^{T5}, if present, is independently selected from: furanyl; thienyl; pyrrolyl; imidazolyl; oxazolyl; isoxazolyl; thiazolyl; isothiazolyl; pyrazolyl; triazolyl; oxadiazolyl; thiadiazolyl; pyridyl; pyridazinyl; pyrimidinyl; pyrazinyl; indolyl; benzimidazolyl; indazolyl; benzofuranyl; benzothienyl; benzoxazolyl; benzothiazolyl; benzisoxazolyl; benzisothiazolyl; quinolinyl; isoquinolinyl; cinnolinyl; quinoxalinyl; quinazolinyl; and phthalazinyl.
(112) A compound according to any one of (1) to (109), wherein each —R^{T5}, if present, is C₅₋₆heteroaryl.
(113) A compound according to any one of (1) to (109), wherein each —R^{T5}, if present, is independently selected from: furanyl; thienyl; pyrrolyl; imidazolyl; oxazolyl; isoxazolyl; thiazolyl; isothiazolyl; pyrazolyl; triazolyl; oxadiazolyl; thiadiazolyl; pyridyl; pyridazinyl; pyrimidinyl; and pyrazinyl.
(114) A compound according to any one of (1) to (109), wherein each —R^{T5}, if present, is independently selected from: furanyl; pyrrolyl; imidazolyl; oxazolyl; isoxazolyl; pyrazolyl; triazolyl; pyridyl; pyridazinyl; pyrimidinyl; and pyrazinyl.
(115) A compound according to any one of (1) to (109), wherein each —R^{T5}, if present, is independently selected from: furanyl; pyrrolyl; imidazolyl; pyrazolyl; pyridyl; pyridazinyl; pyrimidinyl; and pyrazinyl.
(116) A compound according to any one of (1) to (109), wherein each —R^{T5}, if present, is C₉₋₁₂heteroaryl.
(117) A compound according to any one of (1) to (109), wherein each —R^{T5}, if present, is independently selected from: indolyl; benzimidazolyl; indazolyl; benzofuranyl; benzothienyl; benzoxazolyl; benzothiazolyl; benzisoxazolyl; benzisothiazolyl; quinolinyl; isoquinolinyl; cinnolinyl; quinoxalinyl; quinazolinyl; and phthalazinyl.

### The Group -L^{T}-

(118) A compound according to any one of (1) to (117), wherein each -L^{T}-, if present, is independently selected from: —CH₂—, —CH₂CH₂—, —CH(CH₃)—, and —CH₂CH₂CH₂—.
(119) A compound according to any one of (1) to (117), wherein each -L^{T}-, if present, is independently selected from: —CH₂— and —CH₂CH₂—.
(120) A compound according to any one of (1) to (117), wherein each -L^{T}-, if present, is —CH₂—.
Substituents on the Groups —R^{T2}, —R^{T3}, —R^{T4}, and —R^{T}
(121) A compound according to any one of (1) to (120), wherein each —R^{T2}, —R^{T3}, —R^{T4}, and —R^{T5}, if present, is optionally substituted with one or more groups independently selected from:
—R^{TTT}, —R^{TTTX},
—F,
—OH, —OR^{TTT}, and —OR^{TTTX},

### The Group —R^{TTT},

(122) A compound according to any one of (1) to (121), wherein each —R^{TTT}, if present, is independently selected from linear or branched saturated C₁₋₄alkyl, phenyl, and benzyl;
(123) A compound according to any one of (1) to (121), wherein each —R^{TTT}, if present, is independently linear or branched saturated C₁₋₄alkyl.
(124) A compound according to any one of (1) to (121), wherein each —R^{TTT}, if present, is independently linear or branched saturated C₁₋₃alkyl.
(125) A compound according to any one of (1) to (121), wherein each —R^{TTT}, if present, is -Me.

### The Group —R^{TTTX}

(126) A compound according to any one of (1) to (125), wherein each —R^{TTTX}, if present, is independently selected from: —CF₃, —CHF₂, —CH₂CF₃, —CH₂CH₂F, —CH₂CHF₂, —CH(CH₃)CF₃, —CH₂C(CH₃)₂F, —CH₂CF₂CH₃, —CH₂CH₂CF₂CH₃, —CH₂CH₂CHF₂, and —CH₂CH₂CF₃.
(127) A compound according to any one of (1) to (125), wherein each —R^{TTTX}, if present, is independently selected from: —CF₃, —CHF₂, —CH₂CF₃, —CH₂CH₂F, —CH₂CHF₂, —CH(CH₃)CF₃, and —CH₂C(CH₃)₂F.
(128) A compound according to any one of (1) to (125), wherein each —R^{TTTX}, if present, is —CF₃.

### The Group -Q

(129) A compound according to any one of (1) to (128), wherein -Q is:

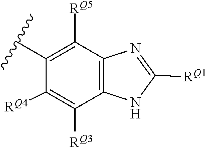

wherein:
—R^{Q1} is independently —H or —R^{QQ1};
—R^{Q3} is independently —H or —R^{QQ3};
—R^{Q4} is independently —H or —R^{QQ4};
—R^{Q5} is independently —H or —R^{QQ5}; and
each of —R^{QQ1}, —R^{QQ3} —R^{QQ4}, and —R^{QQ5} is independently —R^{Q}.
   (130) A compound according to any one of (1) to (128), wherein -Q is:

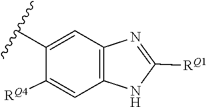

wherein:
—R^{Q1} is independently —H or —R^{QQ1};
—R^{Q4} is independently —H or —R^{QQ4}; and
each of —R^{QQ1} and —R^{QQ4} is independently —R^{Q}.
   (131) A compound according to any one of (1) to (128), wherein -Q is:

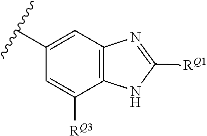

wherein:
—R^{Q1} is independently —H or —R^{QQ1};
—R^{Q3} is independently —H or —R^{QQ3}; and
each of —R^{QQ1} and —R^{QQ3} is independently —R^{Q}.
   (132) A compound according to any one of (1) to (128), wherein -Q is:

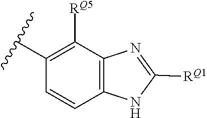

wherein:
—R^{Q1} is independently —H or —R^{QQ1};
—R^{Q5} is independently —H or —R^{QQ5}; and
each of —R^{QQ1} and —R^{QQ5} is independently —R^{Q}.
   (133) A compound according to any one of (1) to (128), wherein -Q is:

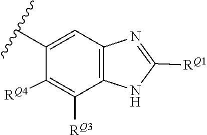

wherein:
—R^{Q1} is independently —H or —R^{QQ1};
—R^{Q3} is independently —H or —R^{QQ3};
—R^{Q4} is independently —H or —R^{QQ4}; and
each of —R^{QQ1}, —R^{QQ3}, and —R^{QQ4} is independently —R^{Q}.
   (134) A compound according to any one of (1) to (128), wherein -Q is:

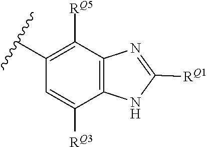

wherein:
—R^{Q1} is independently —H or —R^{QQ1};
—R^{Q3} is independently —H or —R^{QQ3};
—R^{Q5} is independently —H or —R^{QQ5}; and
each of —R^{QQ1}, —R^{QQ3}, and —R^{QQ5} is independently —R^{Q}.
   (135) A compound according to any one of (1) to (128), wherein -Q is:

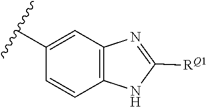

wherein:
—R^{Q1} is independently —H or —R^{QQ1}; and
—R^{QQ1} is independently —R^{Q}.
   (136) A compound according to any one of (1) to (128), wherein -Q is:

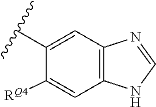

wherein:
—R^{Q4} is independently —H or —R^{QQ4}; and
—R^{QQ4} is independently —R^{Q}.
   (137) A compound according to any one of (1) to (128), wherein -Q is:

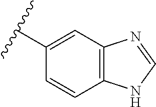 [SMILES: *c1ccc2[nH]cnc2c1]

(138) A compound according to any one of (1) to (128), wherein -Q is:

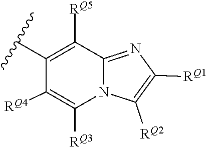

wherein:
—R^{Q1} is independently —H or —R^{QQ1};
—R^{Q2} is independently —H or —R^{QQ2};
—R^{Q3} is independently —H or —R^{QQ3};
—R^{Q4} is independently —H or —R^{QQ4};
—R^{Q5} is independently —H or —R^{QQ5}; and
each of —R^{QQ1}, —R^{QQ2}, —R^{QQ3} —R^{QQ4}, and —R^{QQ5} is independently —R^{Q}.
   (139) A compound according to any one of (1) to (128), wherein -Q is:

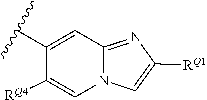

wherein:
—R^{Q1} is independently —H or —R^{QQ1};
—R^{Q4} is independently —H or —R^{QQ4}; and
each of —R^{QQ1} and —R^{QQ4} is independently —R^{Q}.
   (140) A compound according to any one of (1) to (128), wherein -Q is:

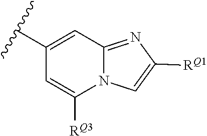

wherein:
—R^{Q1} is independently —H or —R^{QQ1}; and
—R^{Q3} is independently —H or —R^{QQ3};
each of —R^{QQ1} and —R^{QQ3} is independently —R^{Q}.
   (141) A compound according to any one of (1) to (128), wherein -Q is:

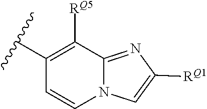

wherein:
—R^{Q1} is independently —H or —R^{QQ1};
—R^{Q5} is independently —H or —R^{QQ5}; and
each of —R^{QQ1} and —R^{QQ5} is independently —R^{Q}.
   (142) A compound according to any one of (1) to (128), wherein -Q is:

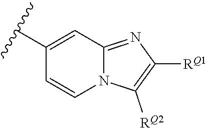

wherein:
—R^{Q1} is independently —H or —R^{QQ1};
—R^{Q2} is independently —H or —R^{QQ2}; and
each of —R^{QQ1} and —R^{QQ2} is independently —R^{Q}.
   (143) A compound according to any one of (1) to (128), wherein -Q is:

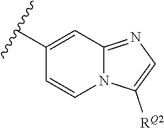

wherein:
—R^{Q2} is independently —H or —R^{QQ2}; and
—R^{QQ2} is independently —R^{Q}.
   (144) A compound according to any one of (1) to (128), wherein -Q is:

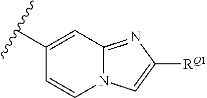

wherein:
—R^{Q1} is independently —H or —R^{QQ1}; and
—R^{QQ1} is independently —R^{Q}.
   (145) A compound according to any one of (1) to (128), wherein -Q is:

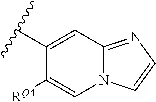

wherein:
—R^{Q4} is independently —H or —R^{QQ4}; and
—R^{QQ4} is independently —R^{Q}.
   (146) A compound according to any one of (1) to (128), wherein -Q is:

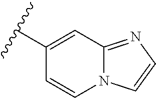 [SMILES: *c1ccn2ccnc2c1]

### The Substituents —R^{Q}

(147) A compound according to any one of (1) to (146), wherein each —R^{Q}, if present, is independently selected from:
—R^{QQ},
—R^{QX},
—F, —Cl, —Br, —I,
—OR^{QQ}, —OR^{QX},
—NH₂, and
—CN.
   (148) A compound according to any one of (1) to (146), wherein each —R^{Q}, if present, is independently selected from:
—R^{QQ},
—F, —Cl,
—OH, —OR^{QQ},
—NH₂.
   (149) A compound according to any one of (1) to (146), wherein each —R^{Q}, if present, is independently selected from:
—F, —Cl,
—OR^{QQ}, and
—NH₂.
   (150) A compound according to any one of (1) to (146), wherein each —R^{Q}, if present, is —R^{QQ}.

### The Group —R^{QQ}

(151) A compound according to any one of (1) to (150), wherein each —R^{QQ}, if present, is independently —R^{QQQ1}.
(152) A compound according to any one of (1) to (150), wherein each —R^{QQ}, if present, is independently —R^{QQQ2}.

### The Group —R^{QQQ1}

(153) A compound according to any one of (1) to (152), wherein each —R^{QQQ1}, if present, is independently linear or branched saturated C₁₋₃alkyl.
(154) A compound according to any one of (1) to (152), wherein each —R^{QQQ1}, if present, is -Me.

### The Group —R^{QQQ2}

(155) A compound according to any one of (1) to (154), wherein each —R^{QQQ2}, if present, is independently selected from: cyclopropyl and cyclobutyl.

### The Group —R^{QX}

(156) A compound according to any one of (1) to (155), wherein each —R^{QX}, if present, is independently selected from: —CF₃, —CHF₂, —CH₂CF₃, —CH₂CH₂F, —CH₂CHF₂, —CH(CH₃)CF₃, —CH₂C(CH₃)₂F, —CH₂CF₂CH₃, —CH₂CH₂CF₂CH₃, —CH₂CH₂CHF₂, and —CH₂CH₂CF₃.
(157) A compound according to any one of (1) to (155), wherein each —R^{QX}, if present, is independently selected from: —CF₃, —CHF₂, —CH₂CF₃, —CH₂CH₂F, —CH₂CHF₂, —CH(CH₃)CF₃, and —CH₂C(CH₃)₂F.
(158) A compound according to any one of (1) to (155), wherein each —R^{QX}, if present, is —CF₃.

### The Position of the Substituents on the Group -J

(159) A compound according to any one of (1) to (158), wherein:
—R^{J1} is —H; and
—R^{J5} is —H.
   (160) A compound according to any one of (1) to (158), wherein:
—R^{J2} is —H; and
—R^{J4} is —H.
   (161) A compound according to any one of (1) to (158), wherein:
—R^{J4} is —H; and
—R^{J5} is —H.
   (162) A compound according to any one of (1) to (158), wherein -J is independently selected from the following groups:

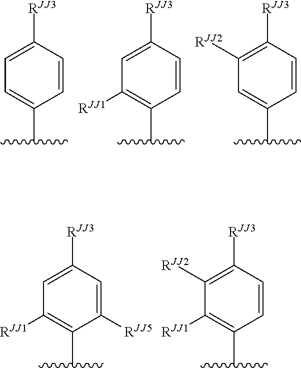

(163) A compound according to any one of (1) to (158), wherein -J is independently selected from the following group:

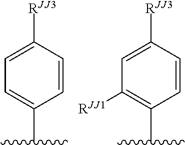

(164) A compound according to any one of (1) to (158), wherein -J is the following group:

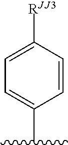

(165) A compound according to any one of (1) to (158), wherein -J is the following group:

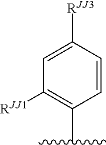

(166) A compound according to any one of (1) to (158), wherein -J is the following group:

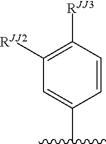

(167) A compound according to any one of (1) to (158), wherein -J is the following group:

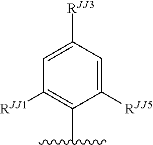

(168) A compound according to any one of (1) to (158), wherein -J is the following group:

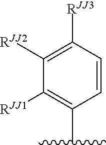

### The Substituents —R^{J}

(169) A compound according to any one of (1) to (168), wherein each —R^{J}, if present, is independently selected from:
—R^{P},
—R^{PX},
—F, —Cl, —Br, —I,
—OH, —OR^{PP}, —OR^{PX},
L^{PP}-OH, -L^{PP}-OR^{PP}, -L^{PP}-OR^{PX},
—NH₂, —NHR^{PP}, —NR^{PP}₂, —NHR^{PX},
L^{PP}-NH₂, -L^{PP}-NHR^{PP}, -L^{PP}-NR^{PP}₂,
—C(═O)R^{PP},
—C(═O)OH, —C(═O)OR^{PP}, —OC(═O)R^{PP},
—C(═O)NH₂, —C(═O)NHR^{PP}, —C(═O)NR^{PP}₂,
—NHC(═O)R^{PP}, —NR^{PN}C(═O)R^{PP},
—S(═O)₂NH₂, —S(═O)₂NHR^{PP}, —S(═O)₂NR^{PP}₂,
—NHS(═O)₂R^{PP}, —NR^{PN}S(═O)₂R^{PP},
—S(═O)R^{PP}, —S(═O)₂R^{PP},
—SH, —SR^{PP}, —SR^{PX},
—CN, and —NO₂.
   (170) A compound according to any one of (1) to (168), wherein each —R^{J}, if present, is independently selected from:
—R^{P},
—R^{PX},
—F, —Cl, —Br,
—OH, —OR^{PP}, —OR^{PX},
—NH₂, —NHR^{PP}, —NR^{PP}₂, —NHR^{PX},
—C(═O)NH₂, —C(═O)NHR^{PP}, —C(═O)NR^{PP}₂,
—NHC(═O)R^{PP},
—S(═O)₂NH₂, —S(═O)₂NHR^{PP}, —S(═O)₂NR^{PP}₂,
—NHS(═O)₂R^{PP},
—S(═O)₂R^{PP},
—SR^{PP}, —SR^{PX},
—CN.

### The Group —R^{JJ1}

(171) A compound according to any one of (1) to (170), wherein —R^{JJ1}, if present, is independently selected from:
—R^{P},
—R^{PX},
—F, —Cl, —Br,
—OH, —OR^{PP}, —OR^{PX},
—NHR^{PP}, —NR^{PP}₂,
—SR^{PP}, —SR^{PX}, and
—CN.
   (172) A compound according to any one of (1) to (170), wherein —R^{JJ1}, if present, is independently selected from:
—R^{P},
—R^{PX},
—F, —Cl, —Br,
—OH, —OR^{PP}, and —OR^{PX}.
   (173) A compound according to any one of (1) to (170), wherein —R^{JJ1}, if present, is independently selected from:
—R^{P},
—F, —Cl,
—OH, and —OR^{PP}.
   (174) A compound according to any one of (1) to (170), wherein —R^{JJ1}, if present, is independently selected from: —R^{P}, —F, and —Cl.
   (175) A compound according to any one of (1) to (170), wherein —R^{JJ1}, if present, is independently —F.

### The Group —R^{JJ5}

(176) A compound according to any one of (1) to (175), wherein —R^{JJ5} if present, is independently selected from:
—R^{P},
—R^{PX},
—F, —Cl, —Br,
—OH, —OR^{PP}, —OR^{PX},
—NHR^{PP}, —NR^{PP}₂,
—SR^{PP}, —SR^{PX}, and
—CN.
   (177) A compound according to any one of (1) to (175), wherein —R^{JJ5} if present, is independently selected from:
—R^{P},
—R^{PX},
—F, —Cl, —Br,
—OH, —OR^{PP}, and —OR^{PX}.
   (178) A compound according to any one of (1) to (175), wherein —R^{JJ5} if present, is independently selected from:
—R^{P},
—F, —Cl,
—OH, and —OR^{PP}.

### The Group —R^{JJ2}

(179) A compound according to any one of (1) to (178), wherein —R^{JJ2}, if present, is independently selected from:
—R^{P},
—R^{PX},
—F, —Cl, —Br,
—OH, —OR^{PP}, —OR^{PX},
—NHR^{PP}, —NR^{PP}₂,
—SR^{PP}, —SR^{PX}, and
—CN.
   (180) A compound according to any one of (1) to (178), wherein —R^{JJ2}, if present, is independently selected from:
—R^{P},
—R^{PX},
—F, —Cl, —Br,
—OH, —OR^{PP}, and —OR^{PX}.
   (181) A compound according to any one of (1) to (178), wherein —R^{JJ2}, if present, is independently selected from:
—R^{P},
—F, —Cl,
—OH, and —OR^{PP}.
   (182) A compound according to any one of (1) to (178), wherein —R^{JJ2}, if present, is independently selected from: —R^{P}, —F, and —Cl.
   (183) A compound according to any one of (1) to (178), wherein —R^{JJ2}, if present, is independently —F.

### The Group —R^{JJ4}

(184) A compound according to any one of (1) to (183), wherein —R^{JJ4}, if present, is independently selected from:
—R^{P},
—R^{PX},
—F, —Cl, —Br,
—OH, —OR^{PP}, —OR^{PX},
—NHR^{PP}, —NR^{PP}₂; and
—CN.
   (185) A compound according to any one of (1) to (183), wherein —R^{JJ4}, if present, is independently selected from:
—R^{P},
—R^{PX},
—F, —Cl, —Br,
—OH, —OR^{PP}, and —OR^{PX}.
   (186) A compound according to any one of (1) to (183), wherein —R^{JJ4}, if present, is independently selected from:
—R^{P},
—F, —Cl,
—OH, and —OR^{PP}.

### The Group —R^{JJ3}

(187) A compound according to any one of (1) to (186), wherein —R^{JJ3}, if present, is independently selected from:
—R^{P},
—R^{PX},
—F, —Cl, —Br,
—OH, —OR^{PP}, —OR^{PX},
—NH₂, —NHR^{PP}, —NR^{PP}₂, —NHR^{PX},
—C(═O)NH₂, —C(═O)NHR^{PP}, —C(═O)NR^{PP}₂,
—NHC(═O)R^{PP},
—S(═O)₂NH₂, —S(═O)₂NHR^{PP}, —S(═O)₂NR^{PP}₂,
—NHS(═O)₂R^{PP},
—S(═O)₂R^{PP},
—SR^{PP}, —SR^{PX},
—CN.
   (188) A compound according to any one of (1) to (186), wherein —R^{JJ3}, if present, is independently selected from:
—R^{P},
—R^{PX},
—OH, —OR^{PP}, —OR^{PX},
—NH₂, —NHR^{PP}, —NR^{PP}₂, —NHR^{PX},
—C(═O)NH₂, —C(═O)NHR^{PP},
—NHC(═O)R^{PP},
—S(═O)₂NH₂, —S(═O)₂NHR^{PP},
—NHS(═O)₂R^{PP},
—S(═O)₂R^{PP},
—SR^{PP}, and —SR^{PX}.
   (189) A compound according to any one of (1) to (186), wherein —R^{JJ3}, if present, is independently selected from:
—R^{P},
—R^{PX},
—OR^{PP}, —OR^{PX},
—NH₂, —NHR^{PP}, —NR^{PP}₂, —NHR^{PX},
—S(═O)₂NH₂, —S(═O)₂NHR^{PP},
—NHS(═O)₂R^{PP},
—S(═O)₂R^{PP},
—SR^{PP}, and —SR^{PX}.
   (190) A compound according to any one of (1) to (186), wherein —R^{JJ3}, if present, is independently selected from: —R^{P}, —OR^{PP}, —OR^{PX}, —NH₂, —NHR^{PP}, and —NR^{PP}₂.
   (191) A compound according to any one of (1) to (186), wherein —R^{JJ3}, if present, is independently selected from: —R^{P}, —OR^{PP}, and —OR^{PX}.
   (192) A compound according to any one of (1) to (186), wherein —R^{JJ3}, if present, is —R^{P}.
   (193) A compound according to any one of (1) to (186), wherein —R^{JJ3}, if present, is independently selected from: —OR^{PP} and —OR^{PX}.
   (194) A compound according to any one of (1) to (186), wherein —R^{JJ3}, if present, is —OR^{PP}.
   (195) A compound according to any one of (1) to (186), wherein —R^{JJ3}, if present, is —OR^{PX}.
   (196) A compound according to any one of (1) to (186), wherein —R^{JJ3}, if present, is independently selected from: —NH₂, —NHR^{PP}, and —NR^{PP}₂.
   (197) A compound according to any one of (1) to (186), wherein —R^{JJ3}, if present, is independently selected from: —NHR^{PP} and —NR^{PP}₂.

### The Group —R^{P}

(198) A compound according to any one of (1) to (197), wherein each —R^{P}, if present, is independently selected from:
—R^{P1}, —R^{P2}, —R^{P3}, —R^{P4} and —R^{P5}.
   (199) A compound according to any one of (1) to (197), wherein each —R^{P}, if present, is independently selected from:
—R^{P1}, —R^{P2}, —R^{P3}, and —R^{P5}.
   (200) A compound according to any one of (1) to (197), wherein each —R^{P}, if present, is independently selected from:
—R^{P1}, —R^{P3}, and —R^{P5}.
   (201) A compound according to any one of (1) to (197), wherein each —R^{P}, if present, is —R^{P1}.
   (202) A compound according to any one of (1) to (197), wherein each —R^{P}, if present, is —R^{P3}.
   (203) A compound according to any one of (1) to (197), wherein each —R^{P}, if present, is —R^{P5}.

### The Group —R^{PP}

(204) A compound according to any one of (1) to (203), wherein each —R^{PP}, if present, is independently selected from:
—R^{P1}, —R^{P2}, —R^{P4}, -LP-R^{P2}, and -LP-R^{P4}.
   (205) A compound according to any one of (1) to (203), wherein each —R^{PP}, if present, is independently selected from:
—R^{P1}, —R^{P2}, and -LP-R^{P2}.
   (206) A compound according to any one of (1) to (203), wherein each —R^{PP}, if present, is —R^{P1}.

### The Group —R^{PX}

(207) A compound according to any one of (1) to (206), wherein each —R^{PX}, if present, is independently selected from: —CF₃, —CHF₂, —CH₂CF₃, —CH₂CH₂F, —CH₂CHF₂, —CH(CH₃)CF₃, —CH₂C(CH₃)₂F, —CH₂CF₂CH₃, —CH₂CH₂CF₂CH₃, —CH₂CH₂CHF₂, and —CH₂CH₂CF₃.
(208) A compound according to any one of (1) to (206), wherein each —R^{PX}, if present, is independently selected from: —CF₃, —CHF₂, —CH₂CF₃, —CH₂CH₂F, —CH₂CHF₂, —CH(CH₃)CF₃, and —CH₂C(CH₃)₂F.
(209) A compound according to any one of (1) to (206), wherein each —R^{PX}, if present, is —CF₃.

### The Group —R^{PN}

(210) A compound according to any one of (1) to (209), wherein each —R^{PN}, if present, is independently linear or branched saturated C₁₋₃alkyl.
(211) A compound according to any one of (1) to (209), wherein each —R^{PN}, if present, is -Me.

### The Group -L^{PP}-

(212) A compound according to any one of (1) to (211), wherein each -L^{PP}-, if present, is independently selected from: —CH₂—, —CH₂CH₂—, —CH(CH₃)—, and —CH₂CH₂CH₂—.
(213) A compound according to any one of (1) to (211), wherein each -L^{PP}-, if present, is independently selected from: —CH₂— and —CH₂CH₂—.
(214) A compound according to any one of (1) to (211), wherein each -L^{PP}-, if present, is —CH₂—.

### The Group —R^{P1}

(215) A compound according to any one of (1) to (214), wherein each —R^{P1}, if present, is independently linear or branched saturated C₁₋₄alkyl.
(216) A compound according to any one of (1) to (214), wherein each —R^{P1}, if present, is independently linear or branched saturated C₁₋₃alkyl.
(217) A compound according to any one of (1) to (214), wherein each —R^{P1}, if present, is -Me.

### The Group —R^{P2}

(218) A compound according to any one of (1) to (217), wherein each —R^{P2}, if present, is independently selected from: cyclopropyl and cyclobutyl.

### The Group —R^{P3}

(219) A compound according to any one of (1) to (218), wherein each —R^{P3}, if present, is independently selected from: non-aromatic monocyclic C₄₋₇heterocyclyl; non-aromatic bridged C₇₋₉heterocyclyl; and non-aromatic spiro C₇₋₉heterocyclyl.
(220) A compound according to any one of (1) to (218), wherein each —R^{P3}, if present, is independently selected from: oxetanyl; tetrahydrofuranyl; tetrahydropyranyl; oxanyl; dioxanyl; azetidinyl; pyrrolidinyl; piperidinyl; piperazinyl; morpholinyl; thiomorpholinyl, 1,4-thiazinane 1,1-dioxide; azepanyl; oxazepanyl; diazepanyl; 2,5-diazabicyclo[2.2.1]heptane; 6-oxa-3-azabicyclo[3.1.1]heptane; 2-oxa-5-azabicyclo[2.2.1]heptane; 5-oxa-2-azabicyclo[4.1.0]heptane; 8-oxa-3-azabicyclo[3.2.1]octane; 3-oxa-8-azabicyclo[3.2.1]octane; 4-oxa-7-azabicyclo[3.2.0]heptane; 3,3a,4,5,6,6a-hexahydro-1H-furo[3,4-c]pyrrole; 6-oxa-3-azaspiro[3.3]heptane; 8-oxa-2-azaspiro[3.4]octane; 7-oxa-2-azaspiro[3.4]octane; 2-oxa-7-azaspiro[3.4]octane; and 8-oxa-3-azaspiro[4.4]nonane.
(221) A compound according to any one of (1) to (218), wherein each —R^{P3}, if present, is non-aromatic monocyclic C₄₋₇heterocyclyl.
(222) A compound according to any one of (1) to (218), wherein each —R^{P3}, if present, is independently selected from: oxetanyl, tetrahydrofuranyl, tetrahydropyranyl, oxanyl, dioxanyl, azetidinyl, pyrrolidinyl, piperidinyl, piperazinyl, morpholinyl, thiomorpholinyl, 1,4-thiazinane 1,1-dioxide, azepanyl, oxazepanyl, and diazepanyl.
(223) A compound according to any one of (1) to (218), wherein each —R^{P3}, if present, is independently selected from: azetidinyl, pyrrolidinyl, piperidinyl, piperazinyl, morpholinyl, thiomorpholinyl, azepanyl, and diazepanyl.
(224) A compound according to any one of (1) to (218), wherein each —R^{P3}, if present, is independently selected from: azetidino, pyrrolidino, piperidino, piperazino, morpholino, thiomorpholino, azepano, and diazepano.
(225) A compound according to any one of (1) to (218), wherein each —R^{P3}, if present, is independently selected from: non-aromatic bridged C₇₋₉heterocyclyl and non-aromatic spiro C₇₋₉heterocyclyl.
(226) A compound according to any one of (1) to (218), wherein each —R^{P3}, if present, is independently selected from: 2,5-diazabicyclo[2.2.1]heptane; 6-oxa-3-azabicyclo[3.1.1]heptane; 2-oxa-5-azabicyclo[2.2.1]heptane; 5-oxa-2-azabicyclo[4.1.0]heptane; 8-oxa-3-azabicyclo[3.2.1]octane; 3-oxa-8-azabicyclo[3.2.1]octane; 4-oxa-7-azabicyclo[3.2.0]heptane; 3,3a,4,5,6,6a-hexahydro-1H-furo[3,4-c]pyrrole; 6-oxa-3-azaspiro[3.3]heptane; 8-oxa-2-azaspiro[3.4]octane; 7-oxa-2-azaspiro[3.4]octane; 2-oxa-7-azaspiro[3.4]octane; and 8-oxa-3-azaspiro[4.4]nonane.
(227) A compound according to any one of (1) to (218), wherein each —R^{P3}, if present, is independently selected from N-linked: 2,5-diazabicyclo[2.2.1]heptane; 6-oxa-3-azabicyclo[3.1.1]heptane; 2-oxa-5-azabicyclo[2.2.1]heptane; 5-oxa-2-azabicyclo[4.1.0]heptane; 8-oxa-3-azabicyclo[3.2.1]octane; 3-oxa-8-azabicyclo[3.2.1]octane; 4-oxa-7-azabicyclo[3.2.0]heptane; 3,3a,4,5,6,6a-hexahydro-1H-furo[3,4-c]pyrrole; 6-oxa-3-azaspiro[3.3]heptane; 8-oxa-2-azaspiro[3.4]octane; 7-oxa-2-azaspiro[3.4]octane; 2-oxa-7-azaspiro[3.4]octane; and 8-oxa-3-azaspiro[4.4]nonane.
(228) A compound according to any one of (1) to (218), wherein each —R^{P3}, if present, is independently selected from the following, and is optionally substituted with one or more groups as described herein:

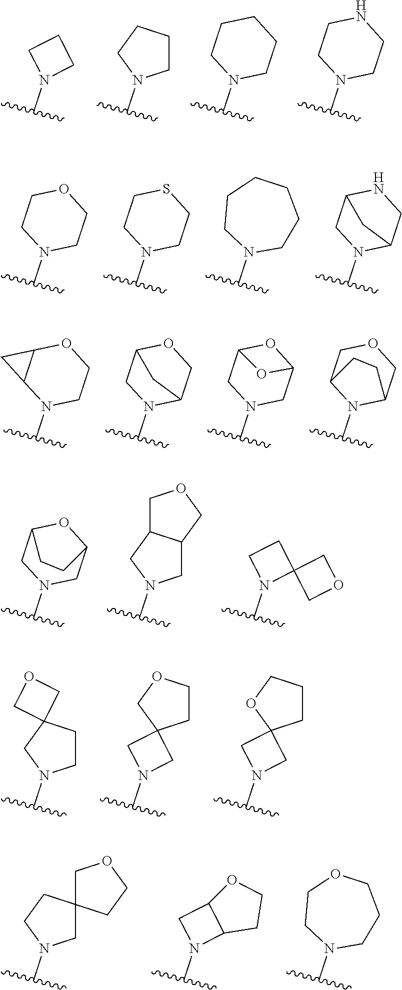

### The Group —R^{P4}

(229) A compound according to any one of (1) to (228), wherein each —R^{P4}, if present, is phenyl.

### The Group —R^{P5}

(230) A compound according to any one of (1) to (229), wherein each —R^{P5}, if present, is independently selected from: C₅₋₆heteroaryl and C₉₋₁₂heteroaryl.
(231) A compound according to any one of (1) to (229), wherein each —R^{P5}, if present, is independently selected from: furanyl; thienyl; pyrrolyl; imidazolyl; oxazolyl; isoxazolyl; thiazolyl; isothiazolyl; pyrazolyl; triazolyl; oxadiazolyl; thiadiazolyl; pyridyl; pyridazinyl; pyrimidinyl; pyrazinyl; indolyl; benzimidazolyl; indazolyl; benzofuranyl; benzothienyl; benzoxazolyl; benzothiazolyl; benzisoxazolyl; benzisothiazolyl; quinolinyl; isoquinolinyl; cinnolinyl; quinoxalinyl; quinazolinyl; and phthalazinyl.
(232) A compound according to any one of (1) to (229), wherein each —R^{P5}, if present, is C₅₋₆heteroaryl.
(233) A compound according to any one of (1) to (229), wherein each —R^{P5}, if present, is independently selected from: furanyl; thienyl; pyrrolyl; imidazolyl; oxazolyl; isoxazolyl; thiazolyl; isothiazolyl; pyrazolyl; triazolyl; oxadiazolyl; thiadiazolyl; pyridyl; pyridazinyl; pyrimidinyl; and pyrazinyl.
(234) A compound according to any one of (1) to (229), wherein each —R^{P5}, if present, is independently selected from: furanyl; pyrrolyl; imidazolyl; oxazolyl; isoxazolyl; pyrazolyl; triazolyl; pyridyl; pyridazinyl; pyrimidinyl; and pyrazinyl.
(235) A compound according to any one of (1) to (229), wherein each —R^{P5}, if present, is independently selected from: furanyl; pyrrolyl; imidazolyl; pyrazolyl; pyridyl; pyridazinyl; pyrimidinyl; and pyrazinyl.
(236) A compound according to any one of (1) to (229), wherein each —R^{P5}, if present, is C₅heteroaryl.
(237) A compound according to any one of (1) to (229), wherein each —R^{P5}, if present, is independently selected from: furanyl; thienyl; pyrrolyl; imidazolyl; oxazolyl; isoxazolyl; thiazolyl; isothiazolyl; pyrazolyl; triazolyl; oxadiazolyl; thiadiazolyl; and tetrazolyl.
(238) A compound according to any one of (1) to (229), wherein each —R^{P5}, if present, is independently selected from: imidazolyl; oxazolyl; isoxazolyl; thiazolyl; isothiazolyl; and pyrazolyl.
(239) A compound according to any one of (1) to (229), wherein each —R^{P5}, if present, is thiazolyl; e.g., thiazol-2-yl; thiazol-4-yl; or thiazol-5-yl.
(240) A compound according to any one of (1) to (229), wherein each —R^{P5}, if present, is independently selected from: thiazol-4-yl; and thiazol-5-yl.
(241) A compound according to any one of (1) to (229), wherein each —R^{P5}, if present, is pyrazolyl; e.g., pyrazol-1-yl; pyrazol-3-yl; pyrazol-4-yl; or pyrazol-5-yl.
(242) A compound according to any one of (1) to (229), wherein each —R^{P5}, if present, is independently selected from: pyrazol-1-yl; and pyrazol-4-yl.
(243) A compound according to any one of (1) to (229), wherein each —R^{P5}, if present, is C₉₋₁₂heteroaryl.
(244) A compound according to any one of (1) to (229), wherein each —R^{P5}, if present, is independently selected from: indolyl; benzimidazolyl; indazolyl; benzofuranyl; benzothienyl; benzoxazolyl; benzothiazolyl; benzisoxazolyl; benzisothiazolyl; quinolinyl; isoquinolinyl; cinnolinyl; quinoxalinyl; quinazolinyl; and phthalazinyl.

### The Group -L^{P}-

(245) A compound according to any one of (1) to (244), wherein each -L^{P}-, if present, is independently selected from: —CH₂—, —CH₂CH₂—, —CH(CH₃)—, and —CH₂CH₂CH₂—.
(246) A compound according to any one of (1) to (244), wherein each -L^{P}-, if present, is independently selected from: —CH₂— and —CH₂CH₂—.
(247) A compound according to any one of (1) to (244), wherein each -L^{P}-, if present, is —CH₂—.
Substituents on the Groups —R^{P2}, —R^{P3}, —R^{P4}, and —R^{P5}
(248) A compound according to any one of (1) to (247), wherein each —R^{P2}, —R^{P3}, —R^{P4}, and —R^{P5}, if present, is optionally substituted with one or more groups independently selected from:
—R^{PPP}, —R^{PPPX},
—F,
—OH, —OR^{PPP}, and —OR^{PPPX};
   and wherein, additionally, each —R^{P2} and —R^{P3} is optionally substituted with ═O (e.g., one or two ═O).
   (249) A compound according to any one of (1) to (247), wherein each —R^{P3}, if present, is optionally substituted with one or more groups independently selected from: —R^{PPP}, —R^{PPPX}, —F, and ═O.

### The Group —R^{PPP}

(250) A compound according to any one of (1) to (249), each —R^{PPP} is independently selected from linear or branched saturated C₁₋₄alkyl, saturated C₃₋₆cycloalkyl, phenyl, and benzyl.
(251) A compound according to any one of (1) to (249), wherein each —R^{PPP}, if present, is independently linear or branched saturated C₁₋₄alkyl.
(252) A compound according to any one of (1) to (249), wherein each —R^{PPP}, if present, is independently linear or branched saturated C₁₋₃alkyl.
(253) A compound according to any one of (1) to (249), wherein each —R^{PPP}, if present, is -Me.

### The Group —R^{PPPX}

(254) A compound according to any one of (1) to (253), wherein each —R^{PPPX}, if present, is independently selected from: —CF₃, —CHF₂, —CH₂CF₃, —CH₂CH₂F, —CH₂CHF₂, —CH(CH₃)CF₃, —CH₂C(CH₃)₂F, —CH₂CF₂CH₃, —CH₂CH₂CF₂CH₃, —CH₂CH₂CHF₂, and —CH₂CH₂CF₃.
(255) A compound according to any one of (1) to (253), wherein each —R^{PPPX}, if present, is independently selected from: —CF₃, —CHF₂, —CH₂CF₃, —CH₂CH₂F, —CH₂CHF₂, —CH(CH₃)CF₃, and —CH₂C(CH₃)₂F.
(256) A compound according to any one of (1) to (253), wherein each —R^{PPPX}, if present, is —CF₃.

### Some Preferred Combinations of Substituents on -J

(257) A compound according to any one of (1) to (186), wherein —R^{J3} is —R^{JJ3}; and —R^{JJ3} is —R^{P}; and that —R^{P} is —R^{P3}.
(258) A compound according to any one of (1) to (186), wherein —R^{J3} is —R^{JJ3}; and —R^{JJ3} is —R^{P}; and that —R^{P} is —R^{P3}; and:
—R^{P3} is independently selected from: azetidino, pyrrolidino, piperidino, piperazino, morpholino, thiomorpholino, azepano, and diazepano; or
—R^{P3} is independently selected from: from N-linked: 2,5-diazabicyclo[2.2.1]heptane; 6-oxa-3-azabicyclo[3.1.1]heptane; 2-oxa-5-azabicyclo[2.2.1]heptane; 5-oxa-2-azabicyclo[4.1.0]heptane; 8-oxa-3-azabicyclo[3.2.1]octane; 3-oxa-8-azabicyclo[3.2.1]octane; 4-oxa-7-azabicyclo[3.2.0]heptane; 3,3a,4,5,6,6a-hexahydro-1H-furo[3,4-c]pyrrole; 6-oxa-3-azaspiro[3.3]heptane; 8-oxa-2-azaspiro[3.4]octane; 7-oxa-2-azaspiro[3.4]octane; 2-oxa-7-azaspiro[3.4]octane; and 8-oxa-3-azaspiro[4.4]nonane.
   (259) A compound according to any one of (1) to (186), wherein —R^{J3} is —R^{JJ3}; and —R^{JJ3} is —R^{P}; and that —R^{P} is —R^{P3}; and —R^{P3} is independently selected from the following, (and is optionally substituted with one or more groups as described herein):

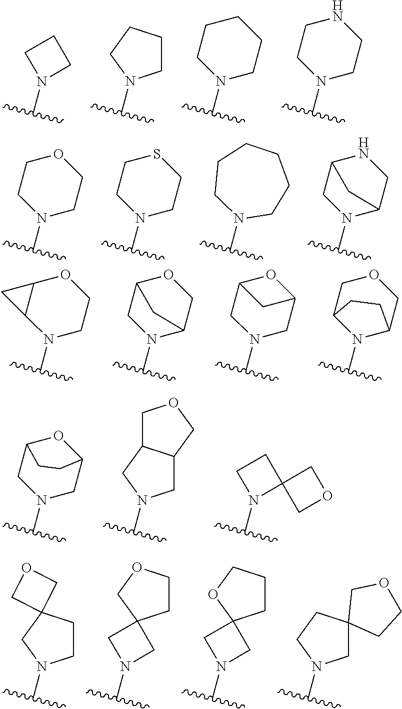

(260) A compound according to any one of (1) to (186), wherein —R^{J3} is —R^{JJ3}; and —R^{JJ3} is —R^{P}; and that —R^{P} is —R^{P3}; and —R^{P3} is independently selected from the following:

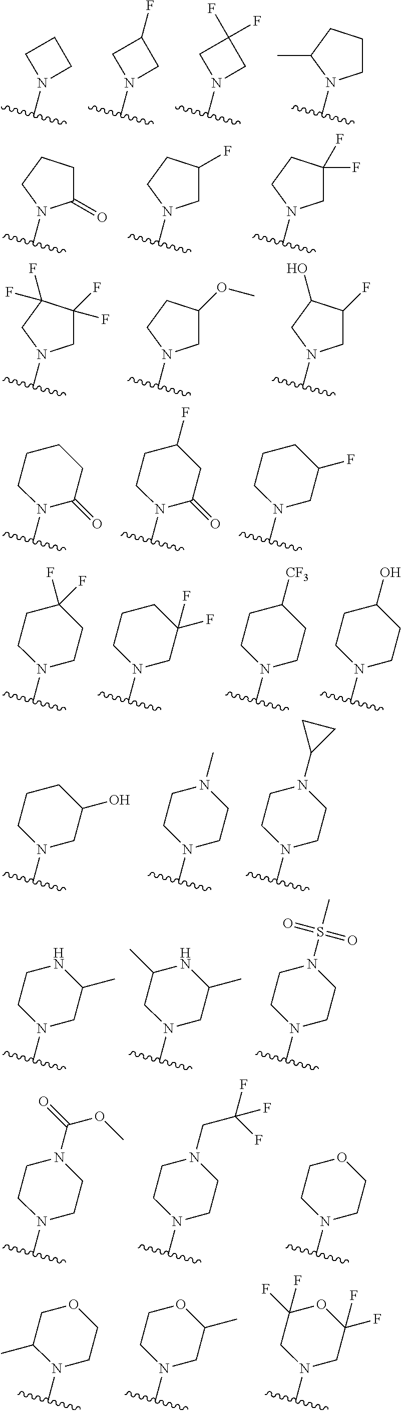

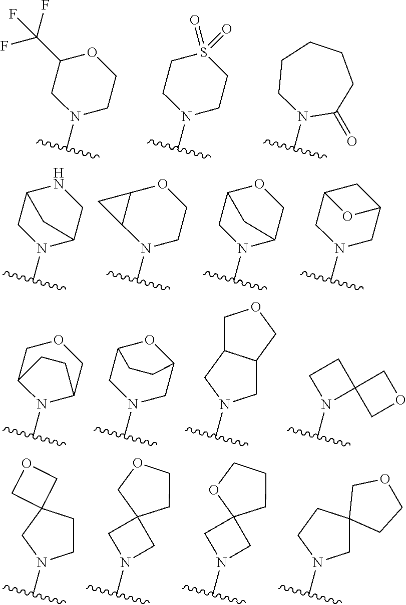

(261) A compound according to any one of (1) to (186), wherein —R^{J3} is —R^{JJ3}; and —R^{JJ3} is —R^{P}; and that —R^{P} is —R^{P5}.
(262) A compound according to any one of (1) to (186), wherein —R^{J3} is —R^{JJ3}; and —R^{JJ3} is —R^{P}; and that —R^{P} is —R^{P5}; and:
—R^{P5} is C₅heteroaryl.
   (263) A compound according to any one of (1) to (186), wherein —R^{J3} is —R^{JJ3}; and —R^{JJ3} is —R^{P}; and that —R^{P} is —R^{P5}; and:
—R^{P5} is independently selected from: furanyl; thienyl; pyrrolyl; imidazolyl; oxazolyl; isoxazolyl; thiazolyl; isothiazolyl; pyrazolyl; triazolyl; oxadiazolyl; thiadiazolyl; and tetrazolyl.
   (264) A compound according to any one of (1) to (186), wherein —R^{J3} is —R^{JJ3}; and —R^{JJ3} is —R^{P}; and that —R^{P} is —R^{P5}; and:
—R^{P5} is independently selected from: imidazolyl; oxazolyl; isoxazolyl; thiazolyl; isothiazolyl; and pyrazolyl.
   (265) A compound according to any one of (1) to (186), wherein —R^{J3} is —R^{JJ3}; and —R^{JJ3} is —R^{P}; and that —R^{P} is —R^{P5}; and:
—R^{P5} is thiazolyl; e.g., thiazol-2-yl; thiazol-4-yl; or thiazol-5-yl.
   (266) A compound according to any one of (1) to (186), wherein —R^{J3} is —R^{JJ3}; and —R^{JJ3} is —R^{P}; and that —R^{P} is —R^{P5}; and:
—R^{P5} is pyrazolyl; e.g., pyrazol-1-yl; pyrazol-3-yl; pyrazol-4-yl; or pyrazol-5-yl.
   (267) A compound according to any one of (1) to (186), wherein —R^{J}3 is —R^{JJ3}; and —R^{JJ3} is —R^{P}; and that —R^{P} is —R^{P5}; and —R^{P5} is independently selected from the following (and is optionally substituted with one or more groups as described herein):

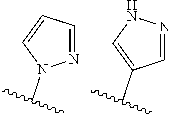

(268) A compound according to any one of (1) to (186), wherein —R^{J3} is —R^{JJ3}; and —R^{JJ3} is —R^{P}; and that —R^{P} is —R^{P5}; and —R^{P5} is independently selected from the following:

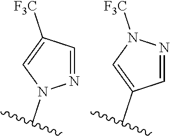

(269) A compound according to any one of (1) to (186), wherein —R^{J3} is —R^{JJ3}; and —R^{JJ3} is —R^{P}; and that —R^{P} is —R^{P5}; and —R^{P5} is independently selected from the following (and is optionally substituted with one or more groups as described herein):

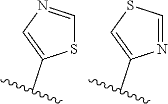

(270) A compound according to any one of (1) to (186), wherein —R^{J3} is —R^{JJ3}; and —R^{JJ3} is —R^{P}; and that —R^{P} is —R^{P5}; and —R^{P5} is independently selected from the following:

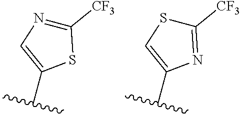 [SMILES: *c1cnc(C(F)(F)F)s1 ; *c1csc(C(F)(F)F)n1]

(271) A compound according to any one of (1) to (186), wherein —R^{J3} is —R^{JJ3}; and —R^{JJ3} is —OR^{PP}; and that —R^{PP} is —R^{P1}.
(272) A compound according to any one of (1) to (186), wherein —R^{J3} is —R^{JJ3}; and —R^{JJ3} is —OR^{PX}.
(273) A compound according to any one of (1) to (186), wherein —R^{J3} is —R^{JJ3}; and R^{JJ3} is —NHR^{PP} and —NR^{PP}₂; and those —R^{PP} groups are —R^{P1} or —R^{P2}.

### Some Preferred Compounds

(274) A compound according to (1), which is a compound of one of the following formulae, or a pharmaceutically acceptable salt, hydrate, or solvate thereof:

| Code No | Chemical Structure |
|---|---|
| FRPPO-001 | 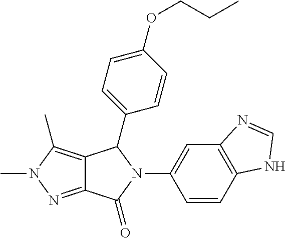 [SMILES: CCCOc1ccc(C2c3c(nn(C)c3C)C(=O)N2c2ccc3[nH]cnc3c2)cc1] |
| FRPPO-002 | 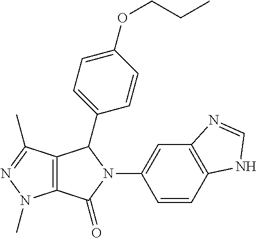 [SMILES: CCCOc1ccc(C2c3c(C)nn(C)c3C(=O)N2c2ccc3[nH]cnc3c2)cc1] |
| FRPPO-003 | 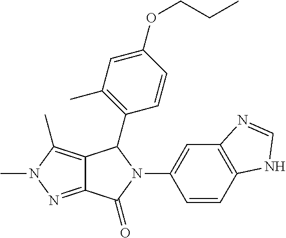 [SMILES: CCCOc1ccc(C2c3c(nn(C)c3C)C(=O)N2c2ccc3[nH]cnc3c2)c(C)c1] |
| FRPPO-004 | 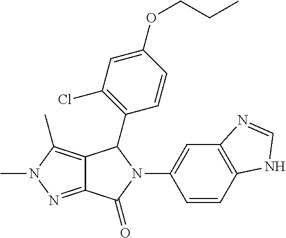 [SMILES: CCCOc1ccc(C2c3c(nn(C)c3C)C(=O)N2c2ccc3[nH]cnc3c2)c(Cl)c1] |
| FRPPO-005 | 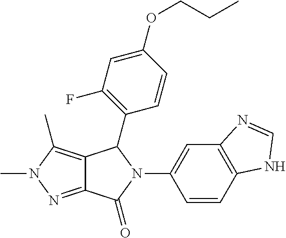 [SMILES: CCCOc1ccc(C2c3c(nn(C)c3C)C(=O)N2c2ccc3[nH]cnc3c2)c(F)c1] |
| FRPPO-006 | 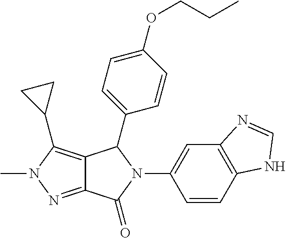 [SMILES: CCCOc1ccc(C2c3c(nn(C)c3C3CC3)C(=O)N2c2ccc3[nH]cnc3c2)cc1] |
| FRPPO-007 | 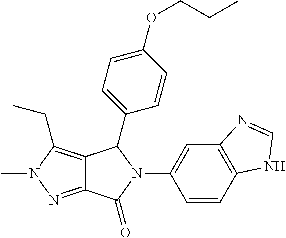 [SMILES: CCCOc1ccc(C2c3c(nn(C)c3CC)C(=O)N2c2ccc3[nH]cnc3c2)cc1] |
| FRPPO-008 | 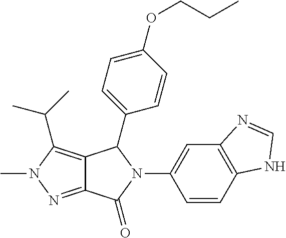 [SMILES: CCCOc1ccc(C2c3c(nn(C)c3C(C)C)C(=O)N2c2ccc3[nH]cnc3c2)cc1] |
| FRPPO-009 | 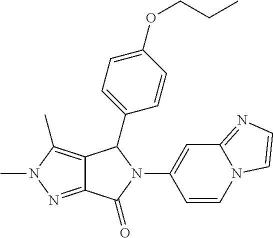 [SMILES: CCCOc1ccc(C2c3c(nn(C)c3C)C(=O)N2c2ccn3ccnc3c2)cc1] |
| FRPPO-010 | 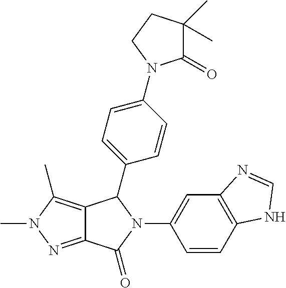 [SMILES: Cc1c2c(nn1C)C(=O)N(c1ccc3[nH]cnc3c1)C2c1ccc(N2CCC(C)(C)C2=O)cc1] |
| FRPPO-011 | 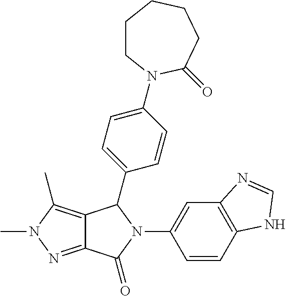 [SMILES: Cc1c2c(nn1C)C(=O)N(c1ccc3[nH]cnc3c1)C2c1ccc(N2CCCCCC2=O)cc1] |
| FRPPO-012 | 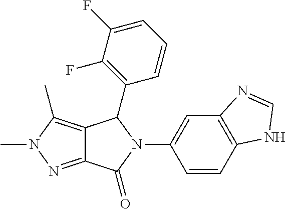 [SMILES: Cc1c2c(nn1C)C(=O)N(c1ccc3[nH]cnc3c1)C2c1cccc(F)c1F] |
| FRPPO-013 | 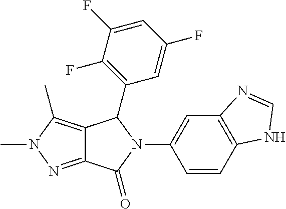 [SMILES: Cc1c2c(nn1C)C(=O)N(c1ccc3[nH]cnc3c1)C2c1cc(F)cc(F)c1F] |
| FRPPO-014 | 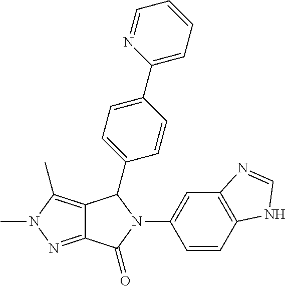 [SMILES: Cc1c2c(nn1C)C(=O)N(c1ccc3[nH]cnc3c1)C2c1ccc(-c2ccccn2)cc1] |
| FRPPO-015 | 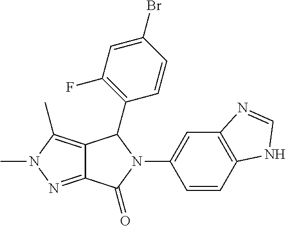 [SMILES: Cc1c2c(nn1C)C(=O)N(c1ccc3[nH]cnc3c1)C2c1ccc(Br)cc1F] |
| FRPPO-016 | 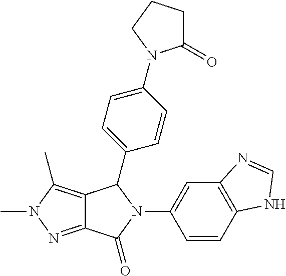 [SMILES: Cc1c2c(nn1C)C(=O)N(c1ccc3[nH]cnc3c1)C2c1ccc(N2CCCC2=O)cc1] |
| FRPPO-017 | 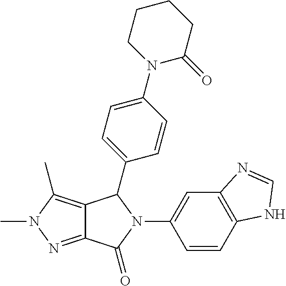 [SMILES: Cc1c2c(nn1C)C(=O)N(c1ccc3[nH]cnc3c1)C2c1ccc(N2CCCCC2=O)cc1] |
| FRPPO-018 | 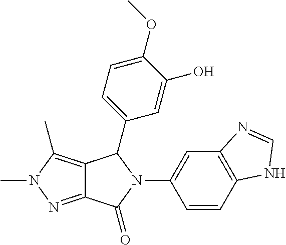 [SMILES: COc1ccc(C2c3c(nn(C)c3C)C(=O)N2c2ccc3[nH]cnc3c2)cc1O] |
| FRPPO-019 | 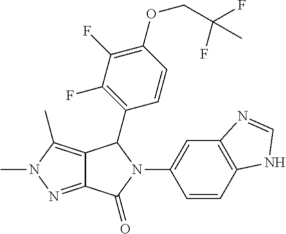 [SMILES: Cc1c2c(nn1C)C(=O)N(c1ccc3[nH]cnc3c1)C2c1ccc(OCC(C)(F)F)c(F)c1F] |
| FRPPO-020 | 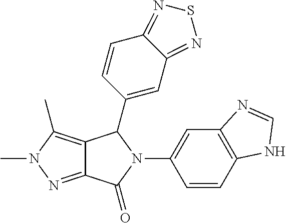 [SMILES: Cc1c2c(nn1C)C(=O)N(c1ccc3[nH]cnc3c1)C2c1ccc2nsnc2c1] |
| FRPPO-021 | 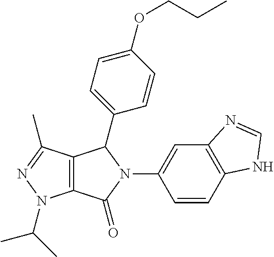 [SMILES: CCCOc1ccc(C2c3c(C)nn(C(C)C)c3C(=O)N2c2ccc3[nH]cnc3c2)cc1] |
| FRPPO-022 | 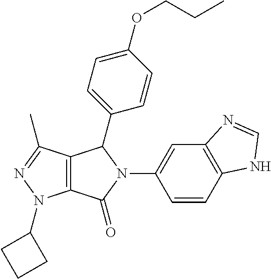 [SMILES: CCCOc1ccc(C2c3c(C)nn(C4CCC4)c3C(=O)N2c2ccc3[nH]cnc3c2)cc1] |
| FRPPO-023 | 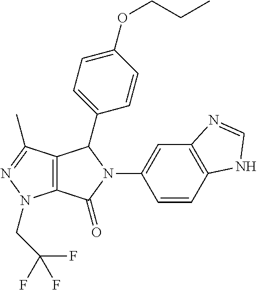 [SMILES: CCCOc1ccc(C2c3c(C)nn(CC(F)(F)F)c3C(=O)N2c2ccc3[nH]cnc3c2)cc1] |
| FRPPO-024 | 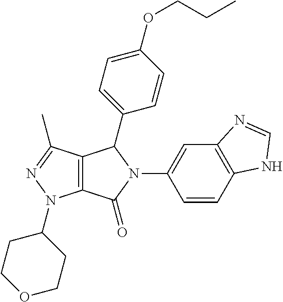 [SMILES: CCCOc1ccc(C2c3c(C)nn(C4CCOCC4)c3C(=O)N2c2ccc3[nH]cnc3c2)cc1] |
| FRPPO-025 | 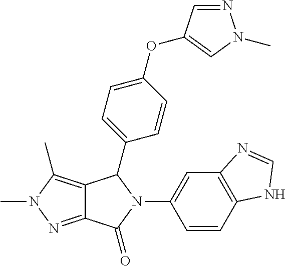 [SMILES: Cc1c2c(nn1C)C(=O)N(c1ccc3[nH]cnc3c1)C2c1ccc(Oc2cnn(C)c2)cc1] |
| FRPPO-026 | 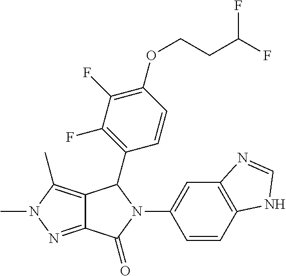 [SMILES: Cc1c2c(nn1C)C(=O)N(c1ccc3[nH]cnc3c1)C2c1ccc(OCCC(F)F)c(F)c1F] |
| FRPPO-027 | 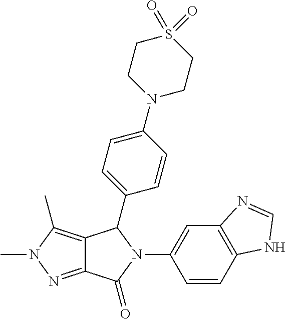 [SMILES: Cc1c2c(nn1C)C(=O)N(c1ccc3[nH]cnc3c1)C2c1ccc(N2CCS(=O)(=O)CC2)cc1] |
| FRPPO-028 | 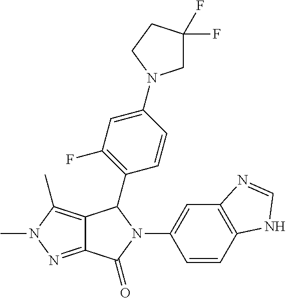 [SMILES: Cc1c2c(nn1C)C(=O)N(c1ccc3[nH]cnc3c1)C2c1ccc(N2CCC(F)(F)C2)cc1F] |
| FRPPO-029 | 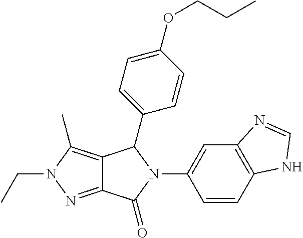 [SMILES: CCCOc1ccc(C2c3c(nn(CC)c3C)C(=O)N2c2ccc3[nH]cnc3c2)cc1] |
| FRPPO-030 | 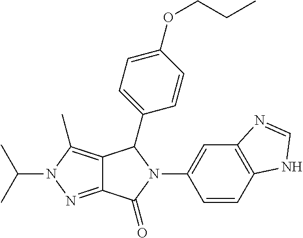 [SMILES: CCCOc1ccc(C2c3c(nn(C(C)C)c3C)C(=O)N2c2ccc3[nH]cnc3c2)cc1] |
| FRPPO-031 | 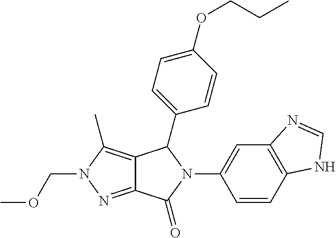 [SMILES: CCCOc1ccc(C2c3c(nn(COC)c3C)C(=O)N2c2ccc3[nH]cnc3c2)cc1] |
| FRPPO-032 | 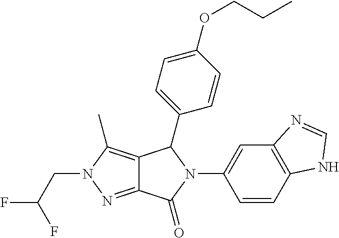 [SMILES: CCCOc1ccc(C2c3c(nn(CC(F)F)c3C)C(=O)N2c2ccc3[nH]cnc3c2)cc1] |
| FRPPO-033 | 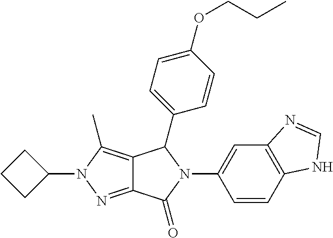 [SMILES: CCCOc1ccc(C2c3c(nn(C4CCC4)c3C)C(=O)N2c2ccc3[nH]cnc3c2)cc1] |
| FRPPO-034 | 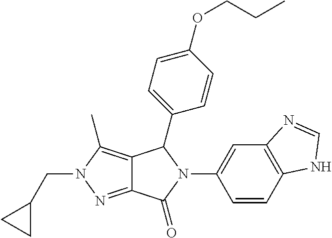 [SMILES: CCCOc1ccc(C2c3c(nn(CC4CC4)c3C)C(=O)N2c2ccc3[nH]cnc3c2)cc1] |
| FRPPO-035 | 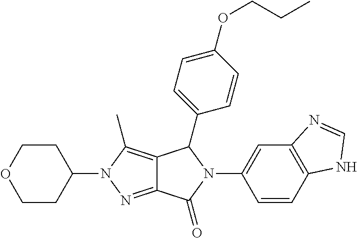 [SMILES: CCCOc1ccc(C2c3c(nn(C4CCOCC4)c3C)C(=O)N2c2ccc3[nH]cnc3c2)cc1] |
| FRPPO-036 | 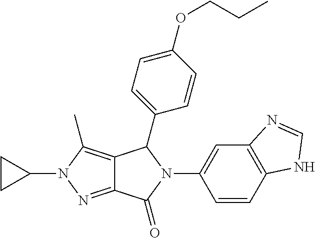 [SMILES: CCCOc1ccc(C2c3c(nn(C4CC4)c3C)C(=O)N2c2ccc3[nH]cnc3c2)cc1] |
| FRPPO-037 | 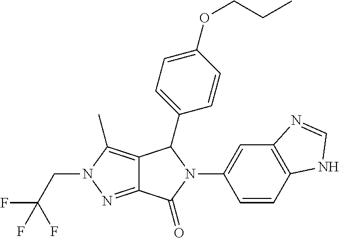 [SMILES: CCCOc1ccc(C2c3c(nn(CC(F)(F)F)c3C)C(=O)N2c2ccc3[nH]cnc3c2)cc1] |
| FRPPO-038 | 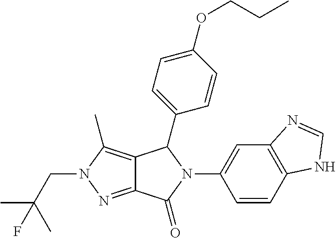 [SMILES: CCCOc1ccc(C2c3c(nn(CC(C)(C)F)c3C)C(=O)N2c2ccc3[nH]cnc3c2)cc1] |
| FRPPO-039 | 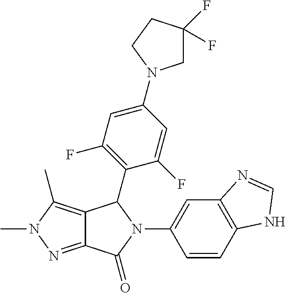 [SMILES: Cc1c2c(nn1C)C(=O)N(c1ccc3[nH]cnc3c1)C2c1c(F)cc(N2CCC(F)(F)C2)cc1F] |
| FRPPO-046 | 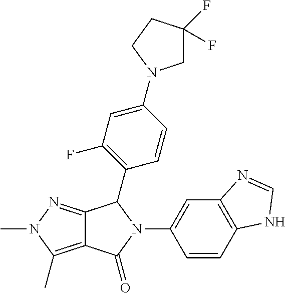 [SMILES: Cc1c2c(nn1C)C(c1ccc(N3CCC(F)(F)C3)cc1F)N(c1ccc3[nH]cnc3c1)C2=O] |
| FRPPO-047 | 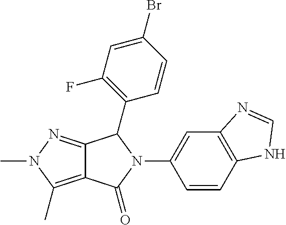 [SMILES: Cc1c2c(nn1C)C(c1ccc(Br)cc1F)N(c1ccc3[nH]cnc3c1)C2=O] |
| FRPPO-048 | 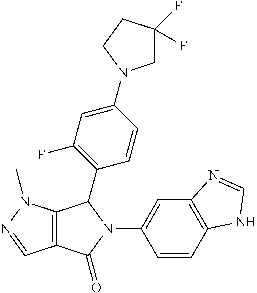 [SMILES: Cn1ncc2c1C(c1ccc(N3CCC(F)(F)C3)cc1F)N(c1ccc3[nH]cnc3c1)C2=O] |
| FRPPO-049 | 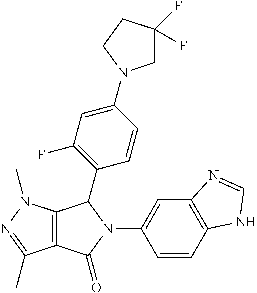 [SMILES: Cc1nn(C)c2c1C(=O)N(c1ccc3[nH]cnc3c1)C2c1ccc(N2CCC(F)(F)C2)cc1F] |
| FRPPO-050 | 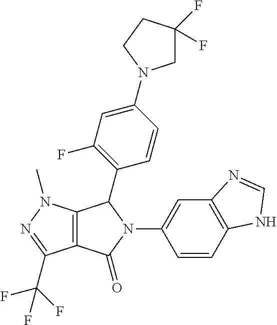 [SMILES: Cn1nc(C(F)(F)F)c2c1C(c1ccc(N3CCC(F)(F)C3)cc1F)N(c1ccc3[nH]cnc3c1)C2=O] |
| FRPPO-051 | 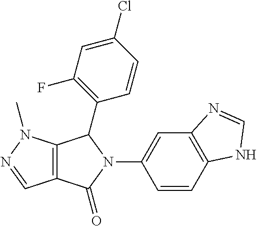 [SMILES: Cn1ncc2c1C(c1ccc(Cl)cc1F)N(c1ccc3[nH]cnc3c1)C2=O] |
| FRPPO-052 | 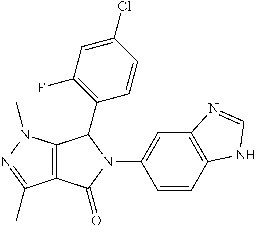 [SMILES: Cc1nn(C)c2c1C(=O)N(c1ccc3[nH]cnc3c1)C2c1ccc(Cl)cc1F] |
| FRPPO-053 | 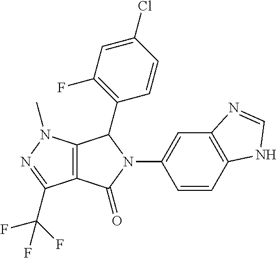 [SMILES: Cn1nc(C(F)(F)F)c2c1C(c1ccc(Cl)cc1F)N(c1ccc3[nH]cnc3c1)C2=O] |
| FRPPO-054 | 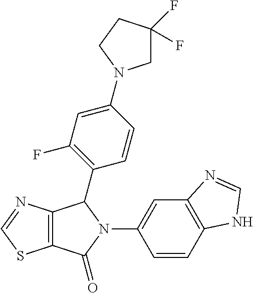 [SMILES: O=C1c2scnc2C(c2ccc(N3CCC(F)(F)C3)cc2F)N1c1ccc2[nH]cnc2c1] |
| FRPPO-057 | 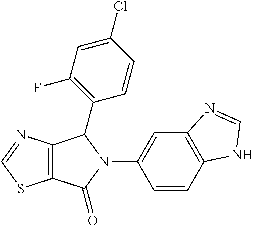 [SMILES: O=C1c2scnc2C(c2ccc(Cl)cc2F)N1c1ccc2[nH]cnc2c1] |
| FRPPO-058 | 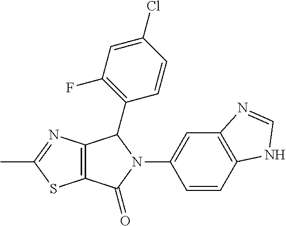 [SMILES: Cc1nc2c(s1)C(=O)N(c1ccc3[nH]cnc3c1)C2c1ccc(Cl)cc1F] |
| FRPPO-061 | 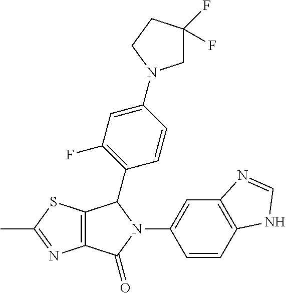 [SMILES: Cc1nc2c(s1)C(c1ccc(N3CCC(F)(F)C3)cc1F)N(c1ccc3[nH]cnc3c1)C2=O] |
| FRPPO-063 | 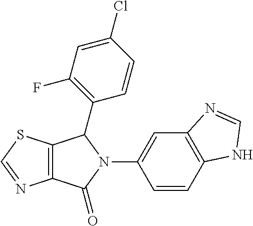 [SMILES: O=C1c2ncsc2C(c2ccc(Cl)cc2F)N1c1ccc2[nH]cnc2c1] |
| FRPPO-064 | 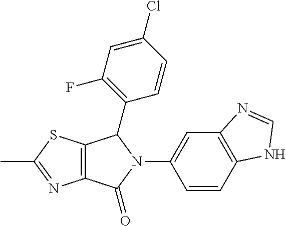 [SMILES: Cc1nc2c(s1)C(c1ccc(Cl)cc1F)N(c1ccc3[nH]cnc3c1)C2=O] |
| FRPPO-066 | 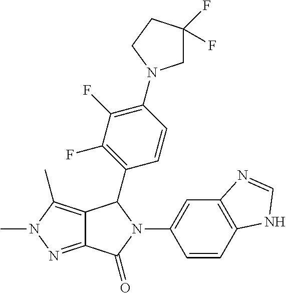 [SMILES: Cc1c2c(nn1C)C(=O)N(c1ccc3[nH]cnc3c1)C2c1ccc(N2CCC(F)(F)C2)c(F)c1F] |
| FRPPO-067 | 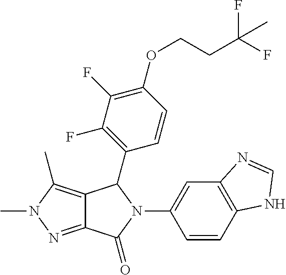 [SMILES: Cc1c2c(nn1C)C(=O)N(c1ccc3[nH]cnc3c1)C2c1ccc(OCCC(C)(F)F)c(F)c1F] |
| FRPPO-068 | 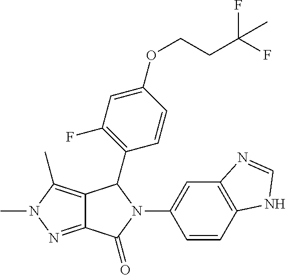 [SMILES: Cc1c2c(nn1C)C(=O)N(c1ccc3[nH]cnc3c1)C2c1ccc(OCCC(C)(F)F)cc1F] |
| FRPPO-069 | 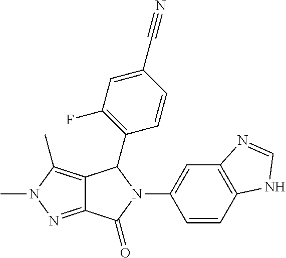 [SMILES: Cc1c2c(nn1C)C(=O)N(c1ccc3[nH]cnc3c1)C2c1ccc(C#N)cc1F] |
| FRPPO-070 | 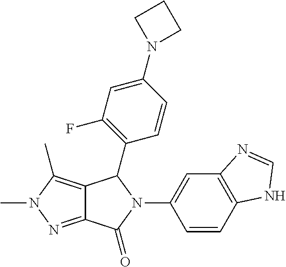 [SMILES: Cc1c2c(nn1C)C(=O)N(c1ccc3[nH]cnc3c1)C2c1ccc(N2CCC2)cc1F] |
| FRPPO-072 | 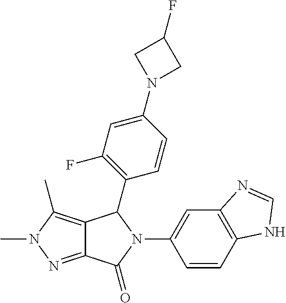 [SMILES: Cc1c2c(nn1C)C(=O)N(c1ccc3[nH]cnc3c1)C2c1ccc(N2CC(F)C2)cc1F] |
| FRPPO-073 | 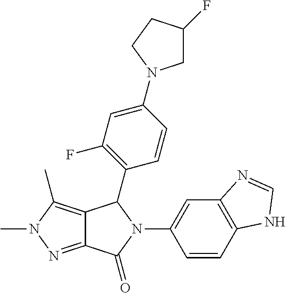 [SMILES: Cc1c2c(nn1C)C(=O)N(c1ccc3[nH]cnc3c1)C2c1ccc(N2CCC(F)C2)cc1F] |
| FRPPO-074 | 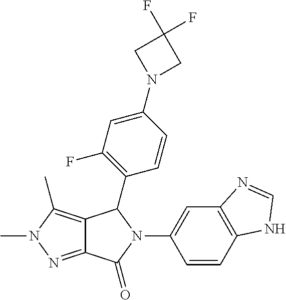 [SMILES: Cc1c2c(nn1C)C(=O)N(c1ccc3[nH]cnc3c1)C2c1ccc(N2CC(F)(F)C2)cc1F] |
| FRPPO-076 | 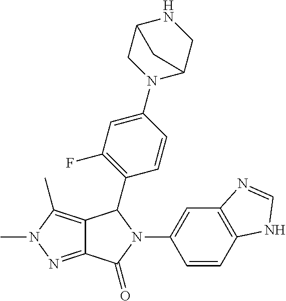 [SMILES: Cc1c2c(nn1C)C(=O)N(c1ccc3[nH]cnc3c1)C2c1ccc(N2CC3CC2CN3)cc1F] |
| FRPPO-077 | 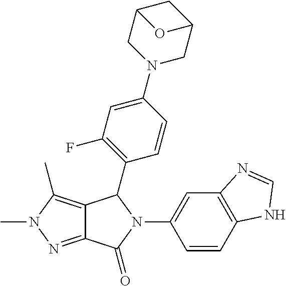 [SMILES: Cc1c2c(nn1C)C(=O)N(c1ccc3[nH]cnc3c1)C2c1ccc(N2CC3CC(C2)O3)cc1F] |
| FRPPO-078 | 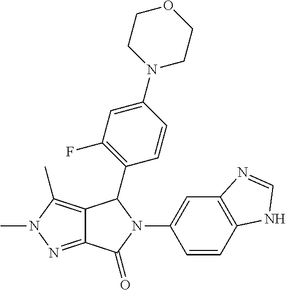 [SMILES: Cc1c2c(nn1C)C(=O)N(c1ccc3[nH]cnc3c1)C2c1ccc(N2CCOCC2)cc1F] |
| FRPPO-079 | 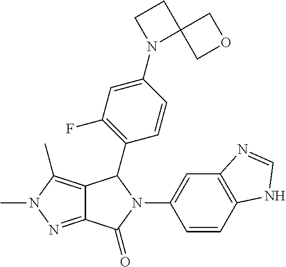 [SMILES: Cc1c2c(nn1C)C(=O)N(c1ccc3[nH]cnc3c1)C2c1ccc(N2CCC23COC3)cc1F] |
| FRPPO-081 | 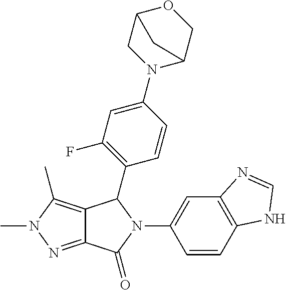 [SMILES: Cc1c2c(nn1C)C(=O)N(c1ccc3[nH]cnc3c1)C2c1ccc(N2CC3CC2CO3)cc1F] |
| FRPPO-082 | 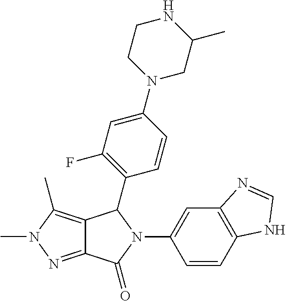 [SMILES: Cc1c2c(nn1C)C(=O)N(c1ccc3[nH]cnc3c1)C2c1ccc(N2CCNC(C)C2)cc1F] |
| FRPPO-083 | 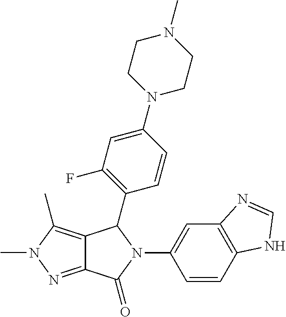 [SMILES: Cc1c2c(nn1C)C(=O)N(c1ccc3[nH]cnc3c1)C2c1ccc(N2CCN(C)CC2)cc1F] |
| FRPPO-084 | 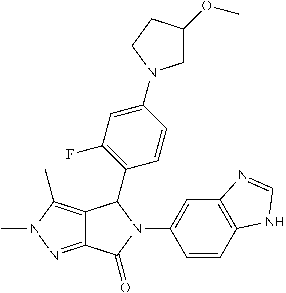 [SMILES: COC1CCN(c2ccc(C3c4c(nn(C)c4C)C(=O)N3c3ccc4[nH]cnc4c3)c(F)c2)C1] |
| FRPPO-085 | 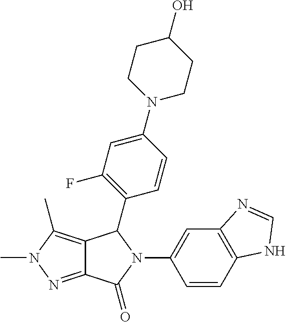 [SMILES: Cc1c2c(nn1C)C(=O)N(c1ccc3[nH]cnc3c1)C2c1ccc(N2CCC(O)CC2)cc1F] |
| FRPPO-086 | 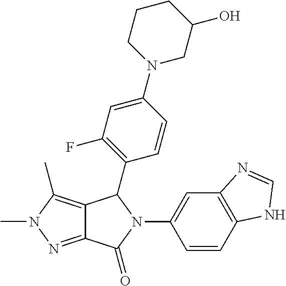 [SMILES: Cc1c2c(nn1C)C(=O)N(c1ccc3[nH]cnc3c1)C2c1ccc(N2CCCC(O)C2)cc1F] |
| FRPPO-087 | 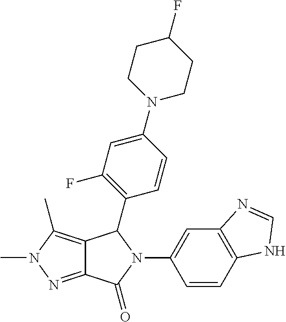 [SMILES: Cc1c2c(nn1C)C(=O)N(c1ccc3[nH]cnc3c1)C2c1ccc(N2CCC(F)CC2)cc1F] |
| FRPPO-088 | 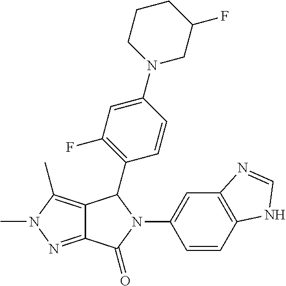 [SMILES: Cc1c2c(nn1C)C(=O)N(c1ccc3[nH]cnc3c1)C2c1ccc(N2CCCC(F)C2)cc1F] |
| FRPPO-089 | 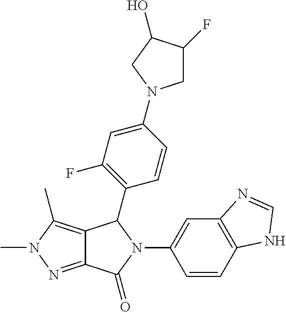 [SMILES: Cc1c2c(nn1C)C(=O)N(c1ccc3[nH]cnc3c1)C2c1ccc(N2CC(O)C(F)C2)cc1F] |
| FRPPO-090 | 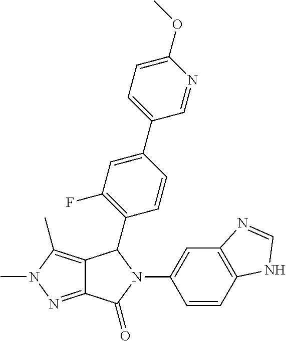 [SMILES: COc1ccc(-c2ccc(C3c4c(nn(C)c4C)C(=O)N3c3ccc4[nH]cnc4c3)c(F)c2)cn1] |
| FRPPO-091 | 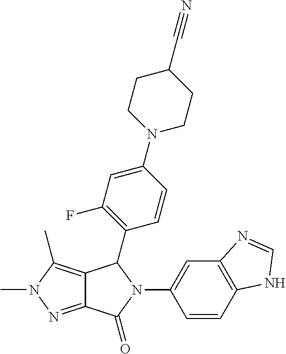 [SMILES: Cc1c2c(nn1C)C(=O)N(c1ccc3[nH]cnc3c1)C2c1ccc(N2CCC(C#N)CC2)cc1F] |
| FRPPO-092 | 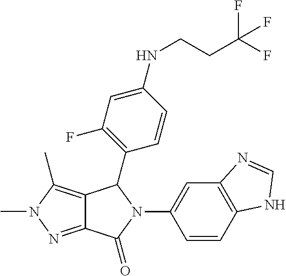 [SMILES: Cc1c2c(nn1C)C(=O)N(c1ccc3[nH]cnc3c1)C2c1ccc(NCCC(F)(F)F)cc1F] |
| FRPPO-093 | 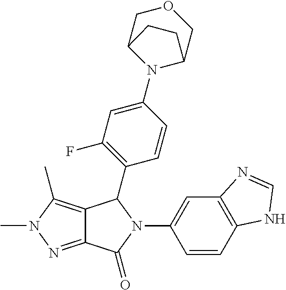 [SMILES: Cc1c2c(nn1C)C(=O)N(c1ccc3[nH]cnc3c1)C2c1ccc(N2C3CCC2COC3)cc1F] |
| FRPPO-094 | 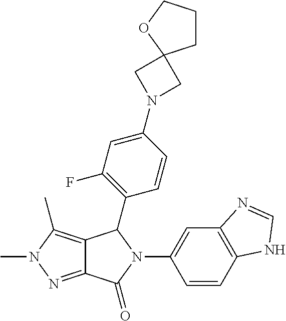 [SMILES: Cc1c2c(nn1C)C(=O)N(c1ccc3[nH]cnc3c1)C2c1ccc(N2CC3(CCCO3)C2)cc1F] |
| FRPPO-095 | 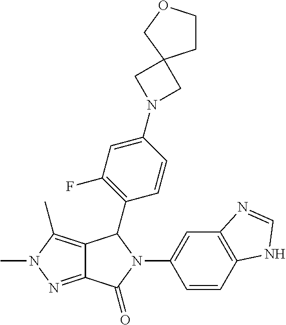 [SMILES: Cc1c2c(nn1C)C(=O)N(c1ccc3[nH]cnc3c1)C2c1ccc(N2CC3(CCOC3)C2)cc1F] |
| FRPPO-096 | 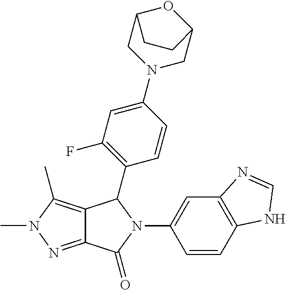 [SMILES: Cc1c2c(nn1C)C(=O)N(c1ccc3[nH]cnc3c1)C2c1ccc(N2CC3CCC(C2)O3)cc1F] |
| FRPPO-097 | 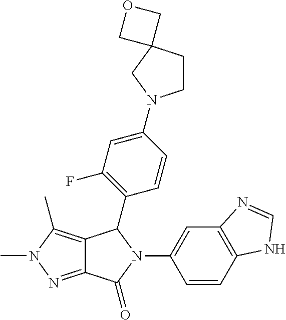 [SMILES: Cc1c2c(nn1C)C(=O)N(c1ccc3[nH]cnc3c1)C2c1ccc(N2CCC3(COC3)C2)cc1F] |
| FRPPO-098 | 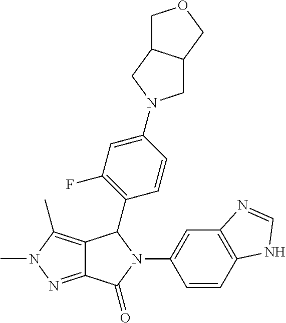 [SMILES: Cc1c2c(nn1C)C(=O)N(c1ccc3[nH]cnc3c1)C2c1ccc(N2CC3COCC3C2)cc1F] |
| FRPPO-100 | 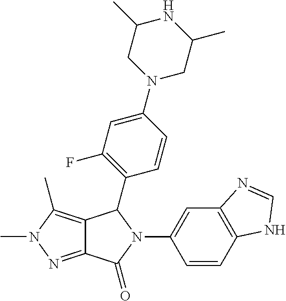 [SMILES: Cc1c2c(nn1C)C(=O)N(c1ccc3[nH]cnc3c1)C2c1ccc(N2CC(C)NC(C)C2)cc1F] |
| FRPPO-101 | 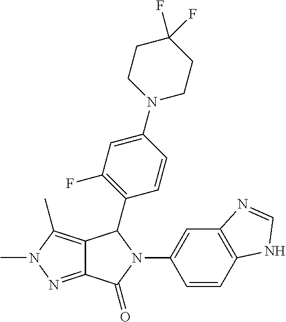 [SMILES: Cc1c2c(nn1C)C(=O)N(c1ccc3[nH]cnc3c1)C2c1ccc(N2CCC(F)(F)CC2)cc1F] |
| FRPPO-102 | 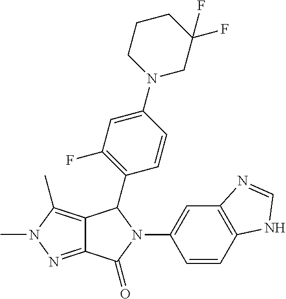 [SMILES: Cc1c2c(nn1C)C(=O)N(c1ccc3[nH]cnc3c1)C2c1ccc(N2CCCC(F)(F)C2)cc1F] |
| FRPPO-103 | 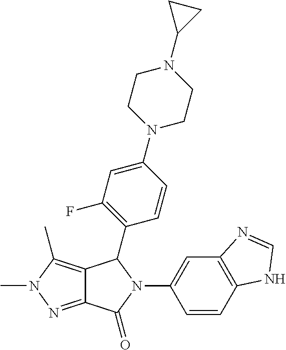 [SMILES: Cc1c2c(nn1C)C(=O)N(c1ccc3[nH]cnc3c1)C2c1ccc(N2CCN(C3CC3)CC2)cc1F] |
| FRPPO-104 | 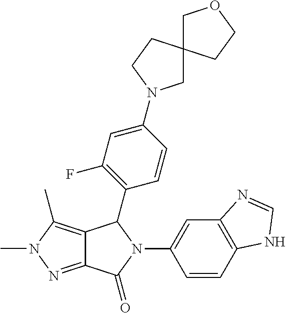 [SMILES: Cc1c2c(nn1C)C(=O)N(c1ccc3[nH]cnc3c1)C2c1ccc(N2CCC3(CCOC3)C2)cc1F] |
| FRPPO-105 | 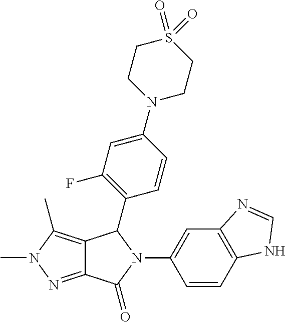 [SMILES: Cc1c2c(nn1C)C(=O)N(c1ccc3[nH]cnc3c1)C2c1ccc(N2CCS(=O)(=O)CC2)cc1F] |
| FRPPO-106 | 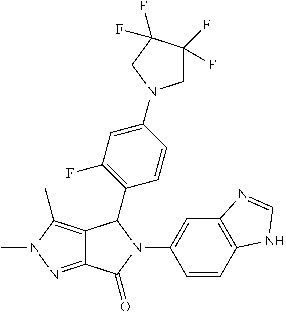 [SMILES: Cc1c2c(nn1C)C(=O)N(c1ccc3[nH]cnc3c1)C2c1ccc(N2CC(F)(F)C(F)(F)C2)cc1F] |
| FRPPO-107 | 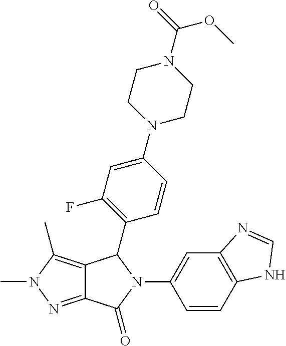 [SMILES: COC(=O)N1CCN(c2ccc(C3c4c(nn(C)c4C)C(=O)N3c3ccc4[nH]cnc4c3)c(F)c2)CC1] |
| FRPPO-108 | 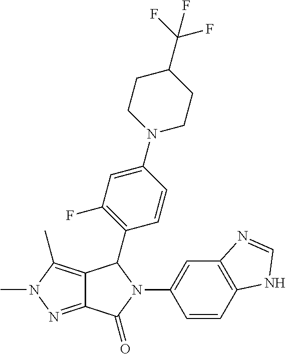 [SMILES: Cc1c2c(nn1C)C(=O)N(c1ccc3[nH]cnc3c1)C2c1ccc(N2CCC(C(F)(F)F)CC2)cc1F] |
| FRPPO-109 | 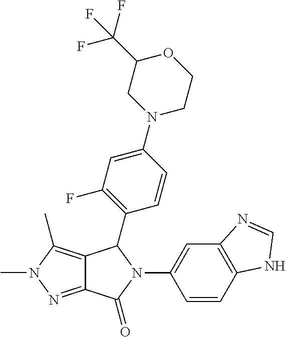 [SMILES: Cc1c2c(nn1C)C(=O)N(c1ccc3[nH]cnc3c1)C2c1ccc(N2CCOC(C(F)(F)F)C2)cc1F] |
| FRPPO-110 | 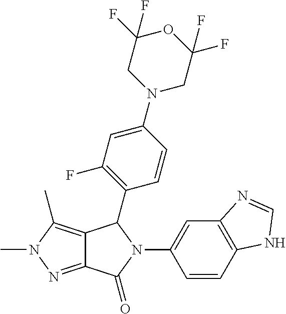 [SMILES: Cc1c2c(nn1C)C(=O)N(c1ccc3[nH]cnc3c1)C2c1ccc(N2CC(F)(F)OC(F)(F)C2)cc1F] |
| FRPPO-111 | 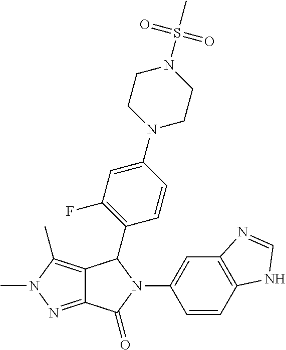 [SMILES: Cc1c2c(nn1C)C(=O)N(c1ccc3[nH]cnc3c1)C2c1ccc(N2CCN(S(C)(=O)=O)CC2)cc1F] |
| FRPPO-112 | 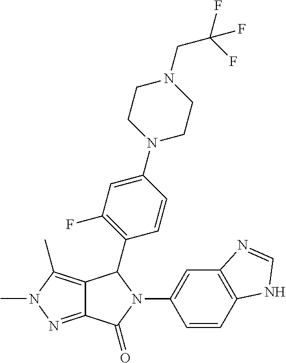 [SMILES: Cc1c2c(nn1C)C(=O)N(c1ccc3[nH]cnc3c1)C2c1ccc(N2CCN(CC(F)(F)F)CC2)cc1F] |
| FRPPO-113 | 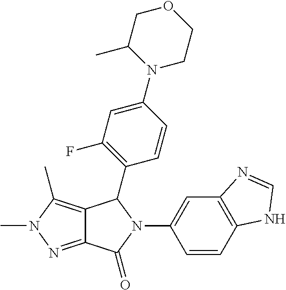 [SMILES: Cc1c2c(nn1C)C(=O)N(c1ccc3[nH]cnc3c1)C2c1ccc(N2CCOCC2C)cc1F] |
| FRPPO-114 | 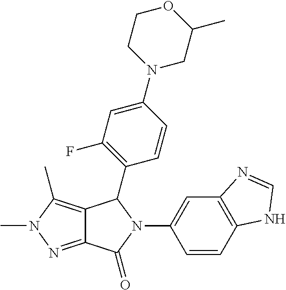 [SMILES: Cc1c2c(nn1C)C(=O)N(c1ccc3[nH]cnc3c1)C2c1ccc(N2CCOC(C)C2)cc1F] |
| FRPPO-115 | 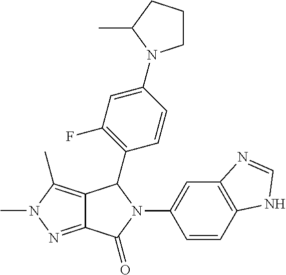 [SMILES: Cc1c2c(nn1C)C(=O)N(c1ccc3[nH]cnc3c1)C2c1ccc(N2CCCC2C)cc1F] |
| FRPPO-126 | 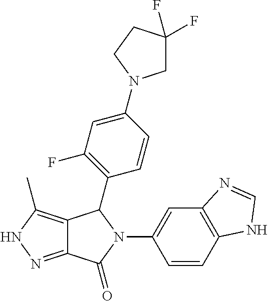 [SMILES: Cc1[nH]nc2c1C(c1ccc(N3CCC(F)(F)C3)cc1F)N(c1ccc3[nH]cnc3c1)C2=O] |
| FRPPO-127 | 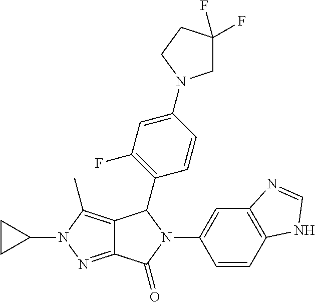 [SMILES: Cc1c2c(nn1C1CC1)C(=O)N(c1ccc3[nH]cnc3c1)C2c1ccc(N2CCC(F)(F)C2)cc1F] |
| FRPPO-134 | 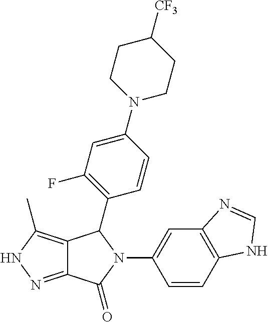 [SMILES: Cc1[nH]nc2c1C(c1ccc(N3CCC(C(F)(F)F)CC3)cc1F)N(c1ccc3[nH]cnc3c1)C2=O] |
| FRPPO-135 | 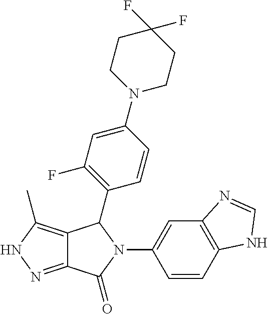 [SMILES: Cc1[nH]nc2c1C(c1ccc(N3CCC(F)(F)CC3)cc1F)N(c1ccc3[nH]cnc3c1)C2=O] |
| FRPPO-136 | 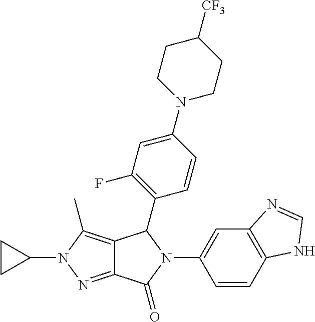 [SMILES: Cc1c2c(nn1C1CC1)C(=O)N(c1ccc3[nH]cnc3c1)C2c1ccc(N2CCC(C(F)(F)F)CC2)cc1F] |
| FRPPO-137 | 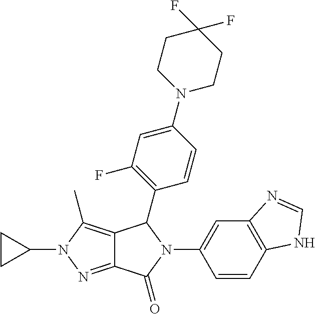 [SMILES: Cc1c2c(nn1C1CC1)C(=O)N(c1ccc3[nH]cnc3c1)C2c1ccc(N2CCC(F)(F)CC2)cc1F] |
| FRPPO-142 | 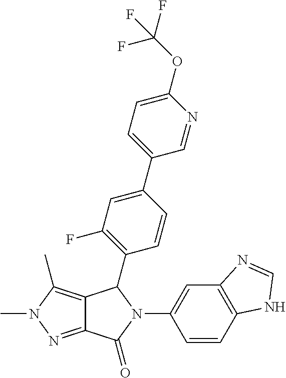 [SMILES: Cc1c2c(nn1C)C(=O)N(c1ccc3[nH]cnc3c1)C2c1ccc(-c2ccc(OC(F)(F)F)nc2)cc1F] |
| FRPPO-143 | 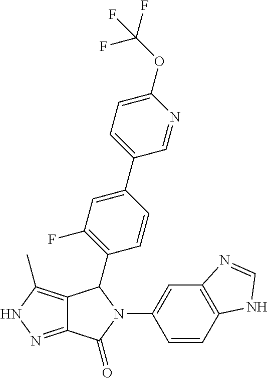 [SMILES: Cc1[nH]nc2c1C(c1ccc(-c3ccc(OC(F)(F)F)nc3)cc1F)N(c1ccc3[nH]cnc3c1)C2=O] |
| FRPPO-144 | 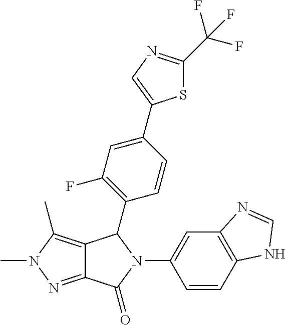 [SMILES: Cc1c2c(nn1C)C(=O)N(c1ccc3[nH]cnc3c1)C2c1ccc(-c2cnc(C(F)(F)F)s2)cc1F] |
| FRPPO-145 | 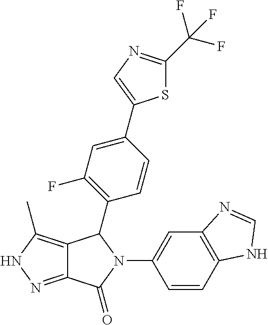 [SMILES: Cc1[nH]nc2c1C(c1ccc(-c3cnc(C(F)(F)F)s3)cc1F)N(c1ccc3[nH]cnc3c1)C2=O] |
| FRPPO-146 | 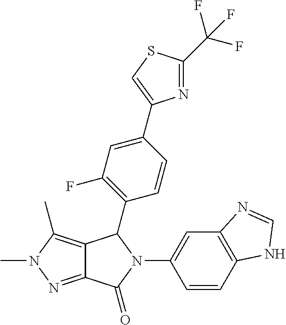 [SMILES: Cc1c2c(nn1C)C(=O)N(c1ccc3[nH]cnc3c1)C2c1ccc(-c2csc(C(F)(F)F)n2)cc1F] |
| FRPPO-147 | 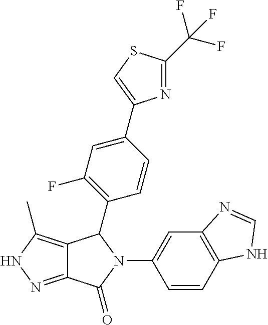 [SMILES: Cc1[nH]nc2c1C(c1ccc(-c3csc(C(F)(F)F)n3)cc1F)N(c1ccc3[nH]cnc3c1)C2=O] |
| FRPPO-148 | 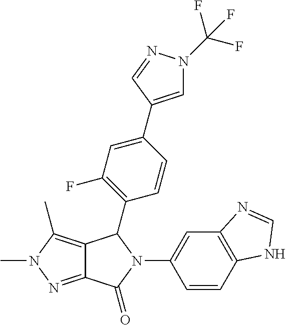 [SMILES: Cc1c2c(nn1C)C(=O)N(c1ccc3[nH]cnc3c1)C2c1ccc(-c2cnn(C(F)(F)F)c2)cc1F] |
| FRPPO-149 | 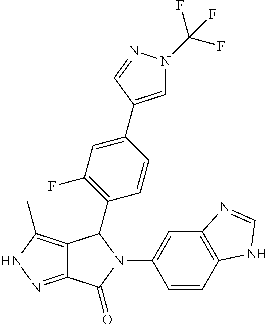 [SMILES: Cc1[nH]nc2c1C(c1ccc(-c3cnn(C(F)(F)F)c3)cc1F)N(c1ccc3[nH]cnc3c1)C2=O] |
| FRPPO-150 | 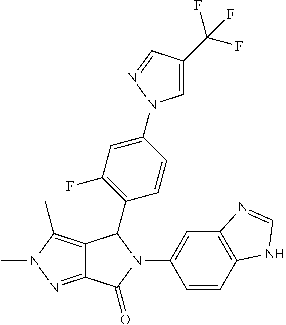 [SMILES: Cc1c2c(nn1C)C(=O)N(c1ccc3[nH]cnc3c1)C2c1ccc(-n2cc(C(F)(F)F)cn2)cc1F] |
| FRPPO-151 | 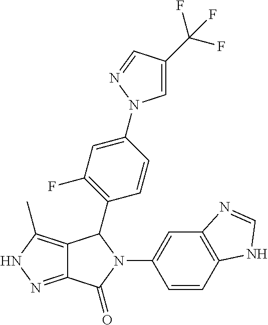 [SMILES: Cc1[nH]nc2c1C(c1ccc(-n3cc(C(F)(F)F)cn3)cc1F)N(c1ccc3[nH]cnc3c1)C2=O] |
| FRPPO-152 | 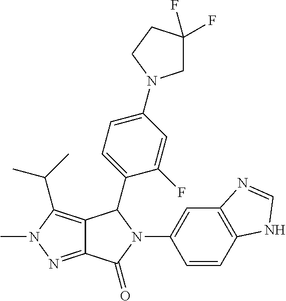 [SMILES: CC(C)c1c2c(nn1C)C(=O)N(c1ccc3[nH]cnc3c1)C2c1ccc(N2CCC(F)(F)C2)cc1F] |
| FRPPO-153 | 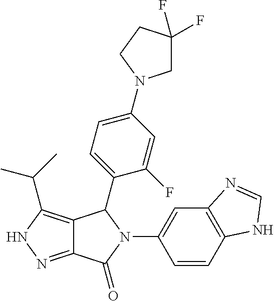 [SMILES: CC(C)c1[nH]nc2c1C(c1ccc(N3CCC(F)(F)C3)cc1F)N(c1ccc3[nH]cnc3c1)C2=O] |
| FRPPO-154 | 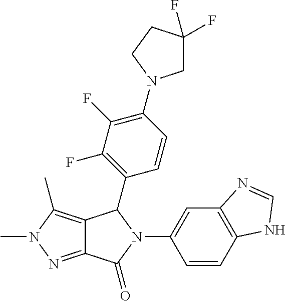 [SMILES: Cc1c2c(nn1C)C(=O)N(c1ccc3[nH]cnc3c1)C2c1ccc(N2CCC(F)(F)C2)c(F)c1F] |
| FRPPO-155 | 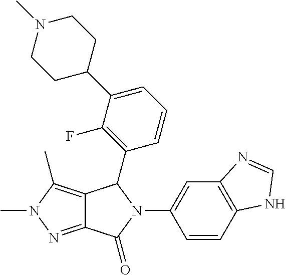 [SMILES: Cc1c2c(nn1C)C(=O)N(c1ccc3[nH]cnc3c1)C2c1cccc(C2CCN(C)CC2)c1F] |
| FRPPO-156 | 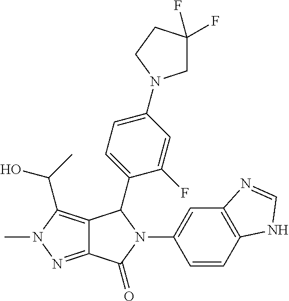 [SMILES: CC(O)c1c2c(nn1C)C(=O)N(c1ccc3[nH]cnc3c1)C2c1ccc(N2CCC(F)(F)C2)cc1F] |
| FRPPO-157 | 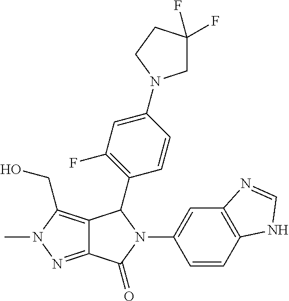 [SMILES: Cn1nc2c(c1CO)C(c1ccc(N3CCC(F)(F)C3)cc1F)N(c1ccc3[nH]cnc3c1)C2=O] |
| FRPPO-158 | 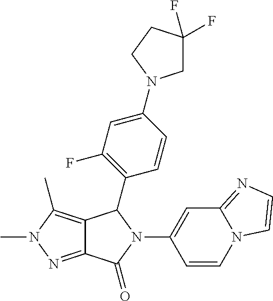 [SMILES: Cc1c2c(nn1C)C(=O)N(c1ccn3ccnc3c1)C2c1ccc(N2CCC(F)(F)C2)cc1F] |
| FRPPO-159 | 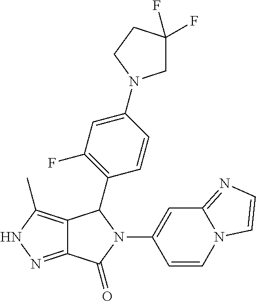 [SMILES: Cc1[nH]nc2c1C(c1ccc(N3CCC(F)(F)C3)cc1F)N(c1ccn3ccnc3c1)C2=O] |
| FRPPO-160 | 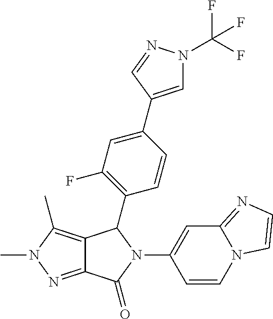 [SMILES: Cc1c2c(nn1C)C(=O)N(c1ccn3ccnc3c1)C2c1ccc(-c2cnn(C(F)(F)F)c2)cc1F] |
| FRPPO-161 | 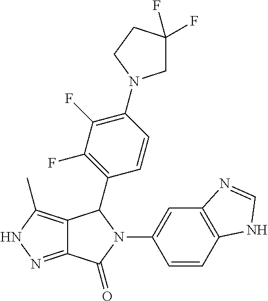 [SMILES: Cc1[nH]nc2c1C(c1ccc(N3CCC(F)(F)C3)c(F)c1F)N(c1ccc3[nH]cnc3c1)C2=O] |
| FRPPO-162 | 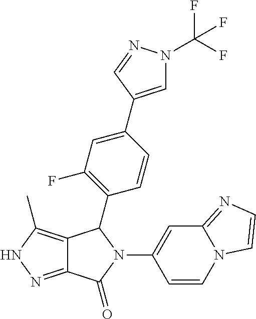 [SMILES: Cc1[nH]nc2c1C(c1ccc(-c3cnn(C(F)(F)F)c3)cc1F)N(c1ccn3ccnc3c1)C2=O] |
| FRPPO-164 | 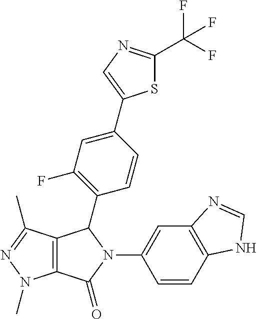 [SMILES: Cc1nn(C)c2c1C(c1ccc(-c3cnc(C(F)(F)F)s3)cc1F)N(c1ccc3[nH]cnc3c1)C2=O] |
| FRPPO-165 | 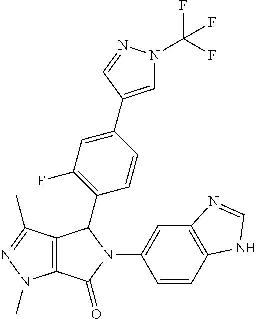 [SMILES: Cc1nn(C)c2c1C(c1ccc(-c3cnn(C(F)(F)F)c3)cc1F)N(c1ccc3[nH]cnc3c1)C2=O] |
| FRPPO-166 | 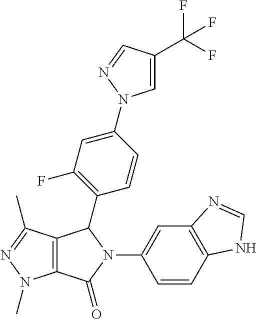 [SMILES: Cc1nn(C)c2c1C(c1ccc(-n3cc(C(F)(F)F)cn3)cc1F)N(c1ccc3[nH]cnc3c1)C2=O] |
| FRPPO-167 | 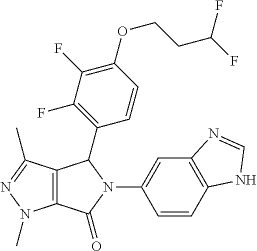 [SMILES: Cc1nn(C)c2c1C(c1ccc(OCCC(F)F)c(F)c1F)N(c1ccc3[nH]cnc3c1)C2=O] |
| FRPPO-168 | 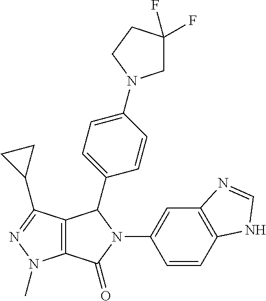 [SMILES: Cn1nc(C2CC2)c2c1C(=O)N(c1ccc3[nH]cnc3c1)C2c1ccc(N2CCC(F)(F)C2)cc1] |
| FRPPO-169 | 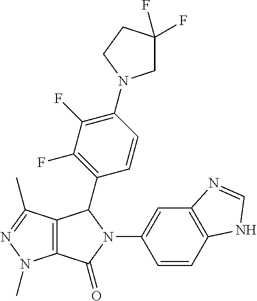 [SMILES: Cc1nn(C)c2c1C(c1ccc(N3CCC(F)(F)C3)c(F)c1F)N(c1ccc3[nH]cnc3c1)C2=O] |
| FRPPO-170 | 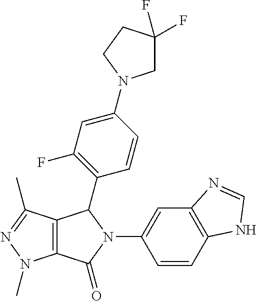 [SMILES: Cc1nn(C)c2c1C(c1ccc(N3CCC(F)(F)C3)cc1F)N(c1ccc3[nH]cnc3c1)C2=O] |
| FRPPO-171 | 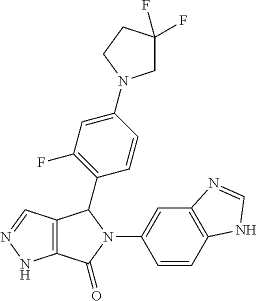 [SMILES: O=C1c2[nH]ncc2C(c2ccc(N3CCC(F)(F)C3)cc2F)N1c1ccc2[nH]cnc2c1] |
| FRPPO-173 | 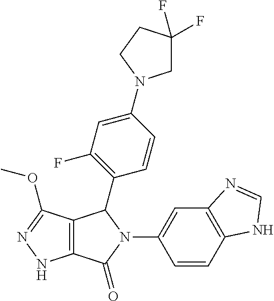 [SMILES: COc1n[nH]c2c1C(c1ccc(N3CCC(F)(F)C3)cc1F)N(c1ccc3[nH]cnc3c1)C2=O] |
| FRPPO-174 | 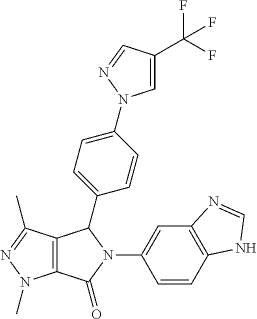 [SMILES: Cc1nn(C)c2c1C(c1ccc(-n3cc(C(F)(F)F)cn3)cc1)N(c1ccc3[nH]cnc3c1)C2=O] |
| FRPPO-175 | 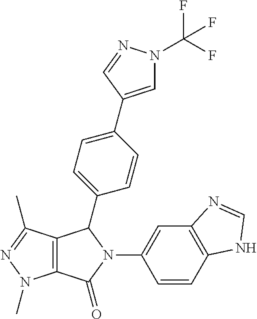 [SMILES: Cc1nn(C)c2c1C(c1ccc(-c3cnn(C(F)(F)F)c3)cc1)N(c1ccc3[nH]cnc3c1)C2=O] |
| FRPPO-176 | 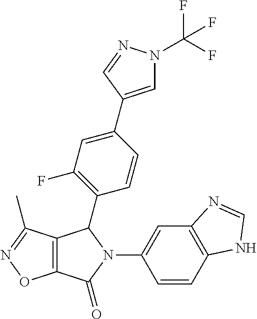 [SMILES: Cc1noc2c1C(c1ccc(-c3cnn(C(F)(F)F)c3)cc1F)N(c1ccc3[nH]cnc3c1)C2=O] |
| FRPPO-177 | 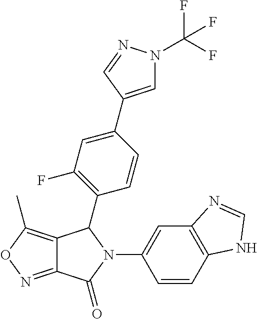 [SMILES: Cc1onc2c1C(c1ccc(-c3cnn(C(F)(F)F)c3)cc1F)N(c1ccc3[nH]cnc3c1)C2=O] |
| FRPPO-197 | 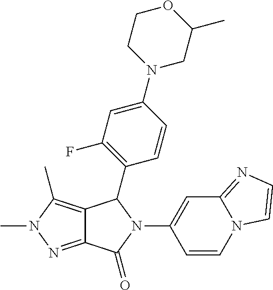 [SMILES: Cc1c2c(nn1C)C(=O)N(c1ccn3ccnc3c1)C2c1ccc(N2CCOC(C)C2)cc1F] |
| FRPPO-198 | 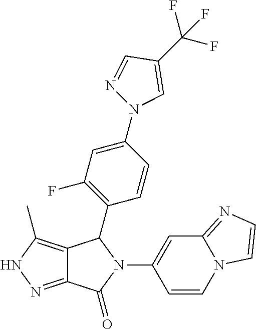 [SMILES: Cc1[nH]nc2c1C(c1ccc(-n3cc(C(F)(F)F)cn3)cc1F)N(c1ccn3ccnc3c1)C2=O] |

### Stereochemistry

(275) A compound according to any one of (1) to (274), wherein the ring atom to which -J is attached, marked with an asterisk (*) in the following formula, is in the (R) configuration:

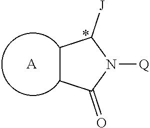

(276) A compound according to any one of (1) to (274), wherein the ring atom to which -J is attached, marked with an asterisk (*) in the following formula, is in the (S) configuration:

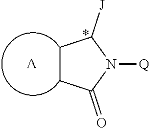

(277) A compound according to any one of (1) to (274), wherein the ring atom to which -J is attached, marked with an asterisk (*) in the following formula, is in the following configuration:

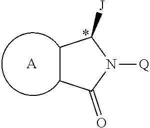

(278) A compound according to any one of (1) to (274), wherein the ring atom to which -J is attached, marked with an asterisk (*) in the following formula, is in the following configuration:

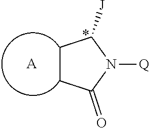

### Combinations

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the chemical groups represented by the variables (e.g., Ring A, -Q, -J, etc.) are specifically embraced by the present invention and are disclosed herein just as if each and every combination was individually and explicitly disclosed, to the extent that such combinations embrace compounds that are stable compounds (i.e., compounds that can be isolated, characterised, and tested for biological activity). In addition, all sub-combinations of the chemical groups listed in the embodiments describing such variables are also specifically embraced by the present invention and are disclosed herein just as if each and every such sub-combination of chemical groups was individually and explicitly disclosed herein.

### Substantially Purified Forms

One aspect of the present invention pertains to FRPPO compounds, as described herein, in substantially purified form and/or in a form substantially free from contaminants.

In one embodiment, the substantially purified form is at least 50% by weight, e.g., at least 60% by weight, e.g., at least 70% by weight, e.g., at least 80% by weight, e.g., at least 90% by weight, e.g., at least 95% by weight, e.g., at least 97% by weight, e.g., at least 98% by weight, e.g., at least 99% by weight.

Unless otherwise specified, the substantially purified form refers to the compound in any stereoisomeric or enantiomeric form. For example, in one embodiment, the substantially purified form refers to a mixture of stereoisomers, i.e., purified with respect to other compounds. In one embodiment, the substantially purified form refers to one stereoisomer, e.g., optically pure stereoisomer. In one embodiment, the substantially purified form refers to a mixture of enantiomers. In one embodiment, the substantially purified form refers to a equimolar mixture of enantiomers (i.e., a racemic mixture, a racemate). In one embodiment, the substantially purified form refers to one enantiomer, e.g., optically pure enantiomer.

In one embodiment, the contaminants represent no more than 50% by weight, e.g., no more than 40% by weight, e.g., no more than 30% by weight, e.g., no more than 20% by weight, e.g., no more than 10% by weight, e.g., no more than 5% by weight, e.g., no more than 3% by weight, e.g., no more than 2% by weight, e.g., no more than 1% by weight. Unless specified, the contaminants refer to other compounds, that is, other than stereoisomers or enantiomers. In one embodiment, the contaminants refer to other compounds and other stereoisomers. In one embodiment, the contaminants refer to other compounds and the other enantiomer.

In one embodiment, the substantially purified form is at least 60% optically pure (i.e., 60% of the compound, on a molar basis, is the desired stereoisomer or enantiomer, and 40% is the undesired stereoisomer or enantiomer), e.g., at least 70% optically pure, e.g., at least 80% optically pure, e.g., at least 90% optically pure, e.g., at least 95% optically pure, e.g., at least 97% optically pure, e.g., at least 98% optically pure, e.g., at least 99% optically pure.

### Isomers

Certain compounds may exist in one or more particular geometric, optical, enantiomeric, diastereoisomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

A reference to a class of structures may well include structurally isomeric forms falling within that class (e.g., C₁₋₇-alkyl includes n-propyl and iso-propyl; butyl includes n-, iso-, sec-, and tert-butyl; methoxyphenyl includes ortho-, meta-, and para-methoxyphenyl). However, reference to a specific group or substitution pattern is not intended to include other structural (or constitutional isomers) which differ with respect to the connections between atoms rather than by positions in space. For example, a reference to a methoxy group, —OCH₃, is not to be construed as a reference to its structural isomer, a hydroxymethyl group, —CH₂OH. Similarly, a reference specifically to ortho-chlorophenyl is not to be construed as a reference to its structural isomer, meta-chlorophenyl.

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro. A reference herein to one tautomer is intended to encompass both tautomers.

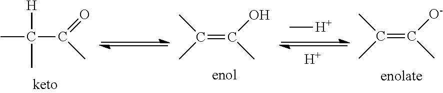

For example, 1H-pyridin-2-one-5-yl and 2-hydroxyl-pyridin-5-yl (shown below) are tautomers of one another. A reference herein to one is intended to encompass both.

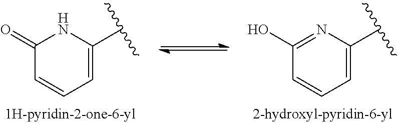 [SMILES: *c1cccc(=O)[nH]1 ; *c1cccc(O)n1]

For example, 1H-benzo[d]imidazol-5-yl and 1H-benzo[d]imidazol-6-yl (shown below) are tautomers of one another. A reference herein to one is intended to encompass both.

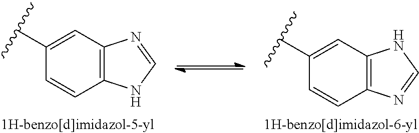 [SMILES: *c1ccc2[nH]cnc2c1 ; *c1ccc2nc[nH]c2c1]

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including mixtures (e.g., racemic mixtures) thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

### Salts

It may be convenient or desirable to prepare, purify, and/or handle a corresponding salt of the compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group, which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺ as well as the ammonium ion (i.e., NH₄⁺). Examples of suitable organic cations include, but are not limited to substituted ammonium ions (e.g., NH₃R⁺, NH₂R₂⁺, NHR₃⁺, NR₄⁺), for example, where each R is independently linear or branched saturated C₁₋₁₈alkyl, C₃₋₈cycloalkyl, C₃₋₈cycloalkyl-C₁₋₆alkyl, and phenyl-C₁₋₆alkyl, wherein the phenyl group is optionally substituted. Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group, which upon protonation may become cationic (e.g., —NH₂ may become —NH₃⁺), then a salt may be formed with a suitable anion.

For example, if a parent structure contains a cationic group (e.g., —NMe₂⁺), or has a functional group, which upon protonation may become cationic (e.g., —NH₂ may become —NH₃⁺), then a salt may be formed with a suitable anion. In the case of a quaternary ammonium compound a counter-anion is generally always present in order to balance the positive charge. If, in addition to a cationic group (e.g., —NMe₂⁺, —NH₃⁺), the compound also contains a group capable of forming an anion (e.g., —COOH), then an inner salt (also referred to as a zwitterion) may be formed.

For example, in the FRPPO compounds described herein, when -Q is a benzimidazolyl group, the —NH— group in the imidazole ring may be protonated, and a salt may be formed with a suitable anion.

Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyloxybenzoic, acetic, trifluoroacetic, ascorbic, aspartic, benzoic, camphorsulfonic, cinnamic, citric, edetic, 1,2-ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Solvates and Hydrates

It may be convenient or desirable to prepare, purify, and/or handle a corresponding solvate of the compound. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, etc.

Unless otherwise specified, a reference to a particular compound also includes solvate and hydrate forms thereof.

### Chemically Protected Forms

It may be convenient or desirable to prepare, purify, and/or handle the compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, reactive chemical reagents, and the like). In practice, well-known chemical methods are employed to reversibly render unreactive a functional group, which otherwise would be reactive, under specified conditions. In a chemically protected form, one or more reactive functional groups are in the form of a protected or protecting group (alternatively as a masked or masking group or a blocked or blocking group). By protecting a reactive functional group, reactions involving other unprotected reactive functional groups can be performed, without affecting the protected group; the protecting group may be removed or the masking group transformed, usually in a subsequent step, without substantially affecting the remainder of the molecule. See, for example, Protective Groups in Organic Synthesis (T. Green and P. Wuts; 4th Edition; John Wiley and Sons, 2006).

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, an amine group may be protected, for example, as an amide (—NRCO—R), for example: as an acetamide (—NHCO—CH₃); or as a carbamate (—NRCO—OR), for example: as a benzyloxy carbamate (—NHCO—OCH₂C₆H₅, —NH-Cbz), as a t-butoxy carbamate (—NHCO—OC(CH₃)₃, —NH-Boc); as a 2-biphenyl-2-propoxy carbamate (—NHCO—OC(CH₃)₂C₆H₄C₆H₅, —NH-Bpoc), as a 9-fluorenylmethoxy carbamate (—NH—Fmoc), as a 6-nitroveratryloxy carbamate (—NH—Nvoc), as a 2-trimethylsilylethyloxy carbamate (—NH-Teoc), a 2,2,2-trichloroethyloxy carbamate (—NH-Troc), as an allyloxy amide (—NH-Alloc), or as a 2(-phenylsulfonyl)ethyloxy carbamate (—NH—Psec); or, in suitable cases (e.g., cyclic amines), as a nitroxide radical (>N—O.); or, in suitable cases (e.g., heterocyclic nitrogens), as a 2-trimethylsilylethoxymethyl (N-SEM).

### Prodrugs

It may be convenient or desirable to prepare, purify, and/or handle the compound in the form of a prodrug. The term “prodrug,” as used herein, pertains to a compound, which yields the desired active compound in vivo. Typically, the prodrug is inactive, or less active than the desired active compound, but may provide advantageous handling, administration, or metabolic properties.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Also, some prodrugs are activated enzymatically to yield the active compound, or a compound, which, upon further chemical reaction, yields the active compound (for example, as in antibody directed enzyme prodrug therapy (ADEPT), gene directed enzyme prodrug therapy (GDEPT), lipid directed enzyme prodrug therapy (LIDEPT), etc.). For example, the prodrug may be a sugar derivative or other glycoside conjugate, or may be an amino acid ester derivative.

### General Chemical Synthesis

Several methods for the chemical synthesis of the FRPPO compounds are described herein. These and/or other well-known methods may be modified and/or adapted in known ways in order to facilitate the synthesis of additional compounds described herein.

In one approach, certain compounds described herein may conveniently be prepared by a multi-component 4,5-dihydropyrrolo[3,4-c]pyrazol-6(2H)-one formation using an aldehyde, amine, 2,4-dioxoester and a hydrazine.

For example, union of a suitably substituted aldehyde with a suitably substituted amine compound, a suitably substituted alkyl 2,4-dioxoester, and a suitably substituted hydrazine gives the corresponding 4,5-dihydropyrrolo[3,4-c]pyrazol-6(2H)-one. This heterocycle formation may be carried out using a variety of conditions, for example, in ethanol and acetic acid or other solvents and acid catalysts.

An example of such a method is illustrated in the following scheme.

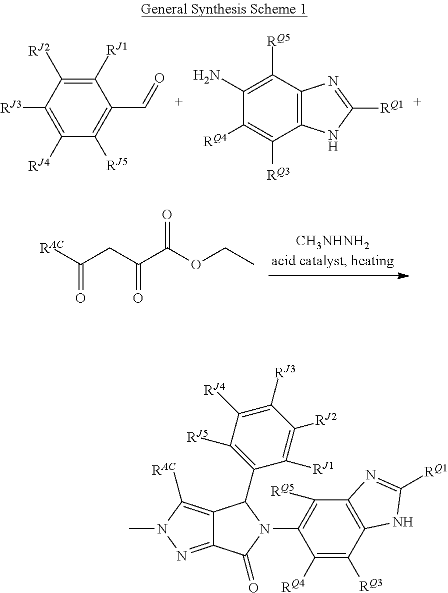

An additional example of such a method is illustrated in the following scheme.

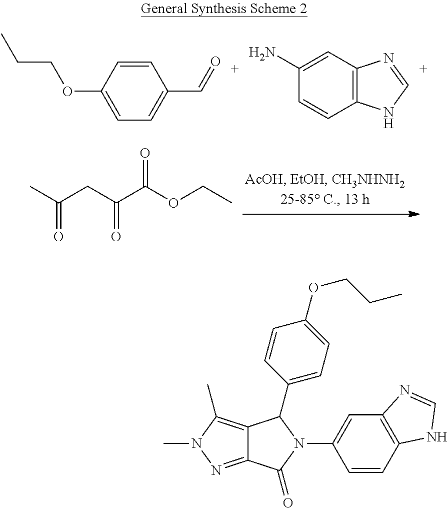

In another approach, certain compounds described herein may conveniently be prepared by a multi-component 4-acyl-3-hydroxy-1,5-dihydro-2H-pyrrol-2-one (or tautomer) formation using an aldehyde, amine and 2,4-dioxoester, followed by condensation with a substituted hydrazine to give a 4,5-dihydropyrrolo[3,4-c]pyrazol-6(2H)-one.

For example, union of a suitably substituted aldehyde with a suitably substituted amine compound and a suitably substituted alkyl 2,4-dioxoester gives the corresponding 4-acyl-3-hydroxy-1,5-dihydro-2H-pyrrol-2-one (or tautomer), e.g., Intermediate X1. This heterocycle formation may be carried out using a variety of conditions, for example, in ethanol and acetic acid or other solvents and acid catalysts. Subsequent reaction with methylhydrazine using an acidic catalyst gives a substituted 4,5-dihydropyrrolo[3,4-c]pyrazol-6(2H)-one.

Examples of such a method are illustrated in the following schemes.

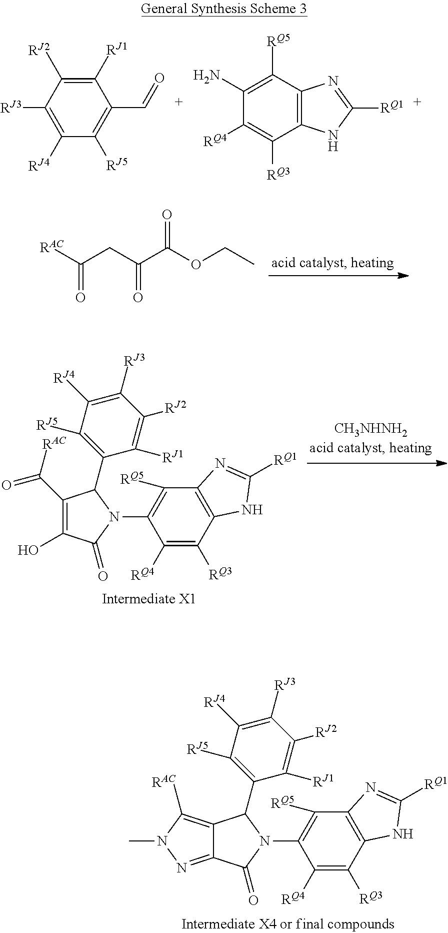

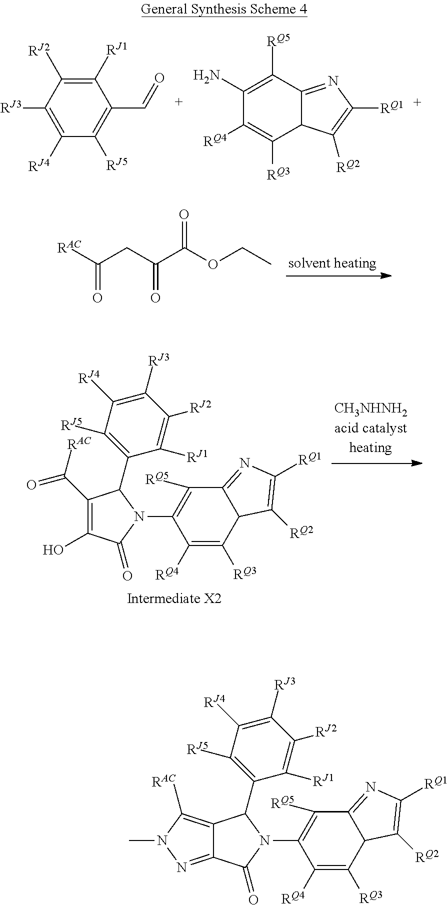

Additional examples of such a method are illustrated in the following schemes.

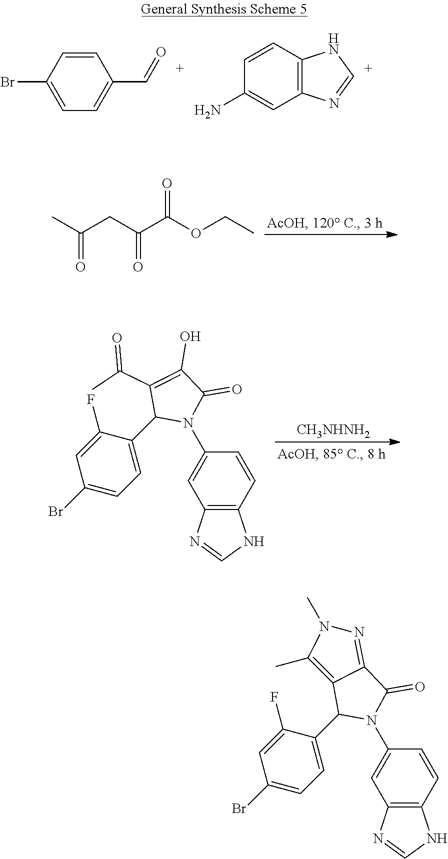

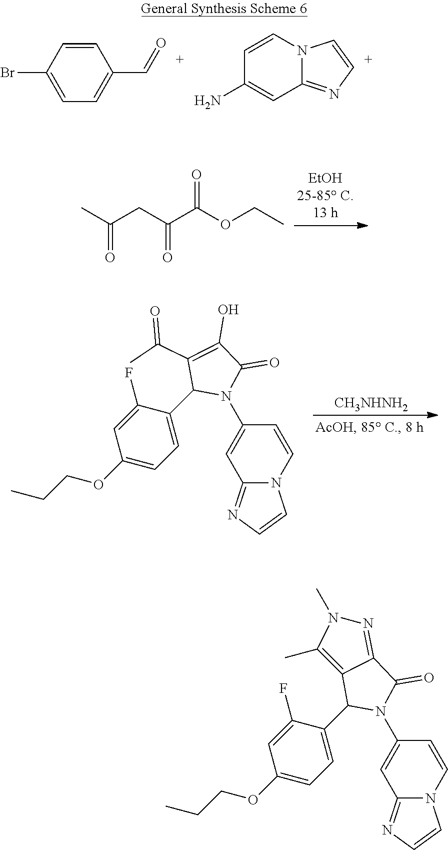

In another approach, certain compounds described herein may conveniently be prepared by reacting a 4-acyl-3-hydroxy-1,5-dihydro-2H-pyrrol-2-one (or tautomer) intermediate with a substituted hydrazine to give a 4,5-dihydropyrrolo[3,4-c]pyrazol-6(2H)-one.

For example, union of a suitably substituted 4-acyl-3-hydroxy-1,5-dihydro-2H-pyrrol-2-one (or tautomer) Intermediate X1 and a suitably substituted hydrazine gives the corresponding 4,5-dihydropyrrolo[3,4-c]pyrazol-6(2H)-one. This heterocycle formation may be carried out using a variety of conditions, for example, in ethanol and acetic acid or other solvents and acid catalysts.

An example of such a method is illustrated in the following scheme.

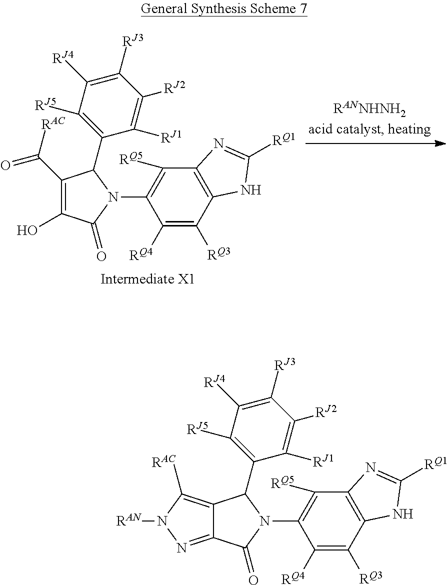

An additional example of such a method is illustrated in the following scheme.

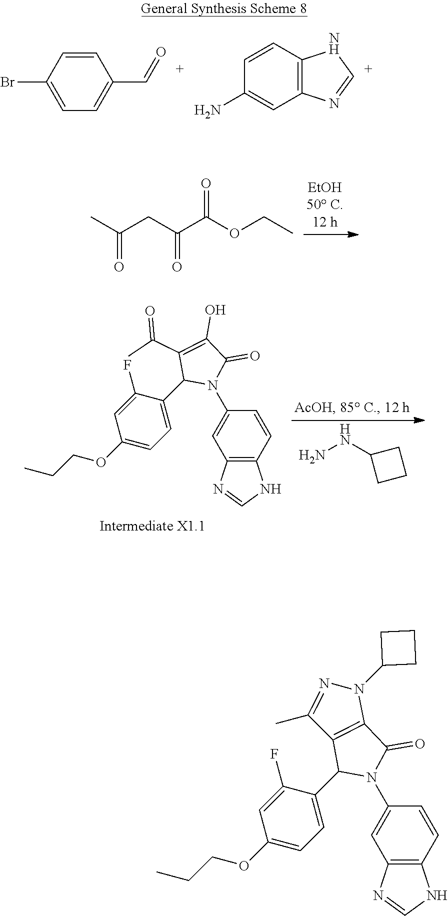 [SMILES: O=Cc1ccc(Br)cc1 ; Nc1ccc2[nH]cnc2c1 ; CCOC(=O)C(=O)CC(C)=O ; CCCOc1ccc(C2C(C(C)=O)=C(O)C(=O)N2c2ccc3[nH]cnc3c2)c(F)c1 ; NNC1CCC1 ; CCCOc1ccc(C2c3c(C)nn(C4CCC4)c3C(=O)N2c2ccc3[nH]cnc3c2)c(F)c1]

In another approach, certain compounds described herein may conveniently be prepared by reacting a 4-acyl-3-hydroxy-1,5-dihydro-2H-pyrrol-2-one (or tautomer) intermediate with methyl hydrazine to give 4,5-dihydropyrrolo[3,4-c]pyrazol-6(2H)-one and 4,5-dihydropyrrolo[3,4-c]pyrazol-6(1H)-one isomers. The isomeric products can be individually separated by chiral chromatography to give the stereoisomers.

For example, union of a suitably substituted 4-acyl-3-hydroxy-1,5-dihydro-2H-pyrrol-2-one (or tautomer) Intermediate X1 and methyl hydrazine gives the corresponding 4,5-dihydropyrrolo[3,4-c]pyrazol-6(2H)-one and 4,5-dihydropyrrolo[3,4-c]pyrazol-6(1H)-one. This heterocycle formation may be carried out using a variety of conditions, for example, in ethanol and acetic acid or other solvents and acid catalysts. Chiral separation gives all four possible region- and stereo-isomers.

An example of such a method is illustrated in the following scheme.

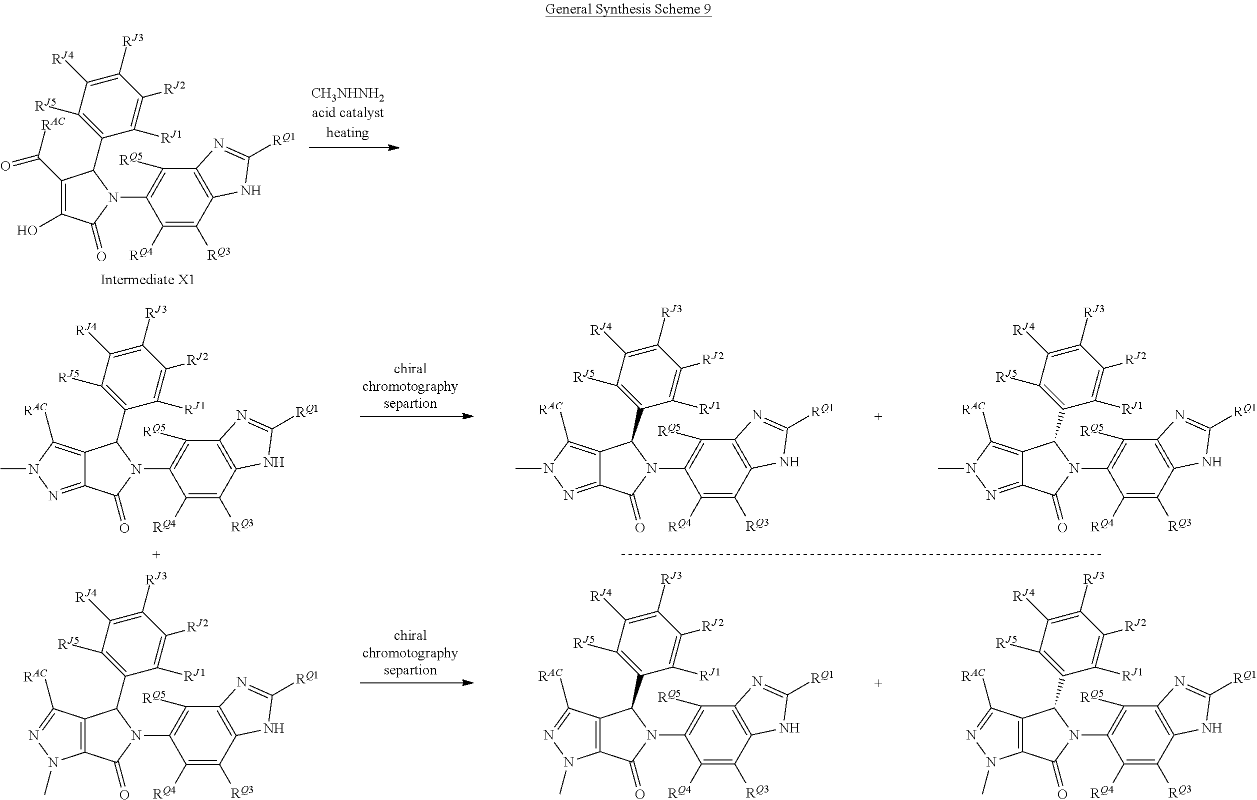

An additional example of such a method is illustrated in the following scheme.

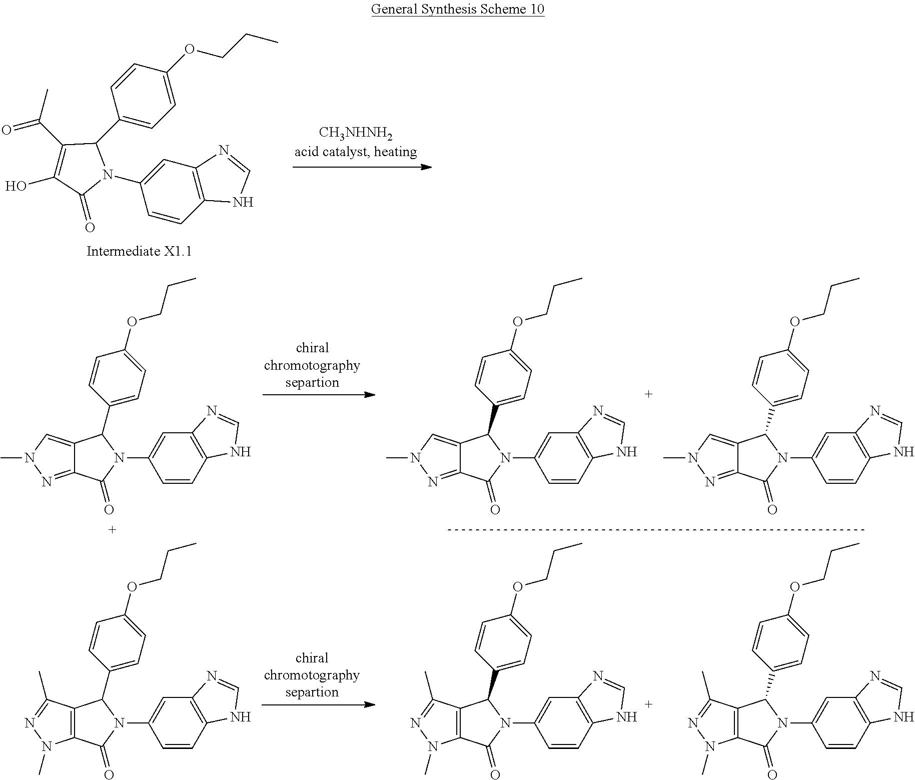

4,5-dihydropyrrolo-fused heterocycles may also be prepared in a synthetic sequence that begins with the halogenation of an ester-substituted heterocycle, followed by metalation of the halogen using metal or organometallic reagents and subsequent reaction with an aldehyde to generate a substituted secondary alcohol. Activation of the hydroxyl group via halogenation or derivatization and displacement with an amine, gives a secondary amine. Hydrolysis of the ester and cyclization with the nascent amines closes the 4,5-dihydropyrrolo-fused ring and furnishes the bicyclic heterocycle. If protecting groups are used on intermediate compounds such as the amines, they can be removed with suitable reagents in the final step.

For example, a suitably substituted alkyl este can be substituted with a halogen such as bromine or iodine using standard electrophilic halogenation reagents such as NBS, NIS or I₂ to give an intermediate such as Intermediate X3. Metal halogen exchange or transmetalation using for example an alkyl lithium, alkyl magnesium or magnesium, and reaction of the resulting heterocyclic organometallic species with a suitably substituted aryl aldehyde gives a secondary alcohol, which in turn is activated by chlorination with SOCl₂, bromination with PBr₃ or sulfonylation with MsCl. Substitution with a suitably substituted aniline under basic conditions, gives an intermediate substituted amino ester such as Intermediate X6. Conversion of the ester to a carboxylic acid using NaOH or similar base, or other suitable reagents, and cyclization using a dehydrating reagent, such as Ghosez's reagent, and a final deprotection step, for example removal of a SEM group with TBAF or TFA, gives a substituted 4,5-dihydropyrrolo-fused heterocycle.

An example of such a method is illustrated in the following scheme.

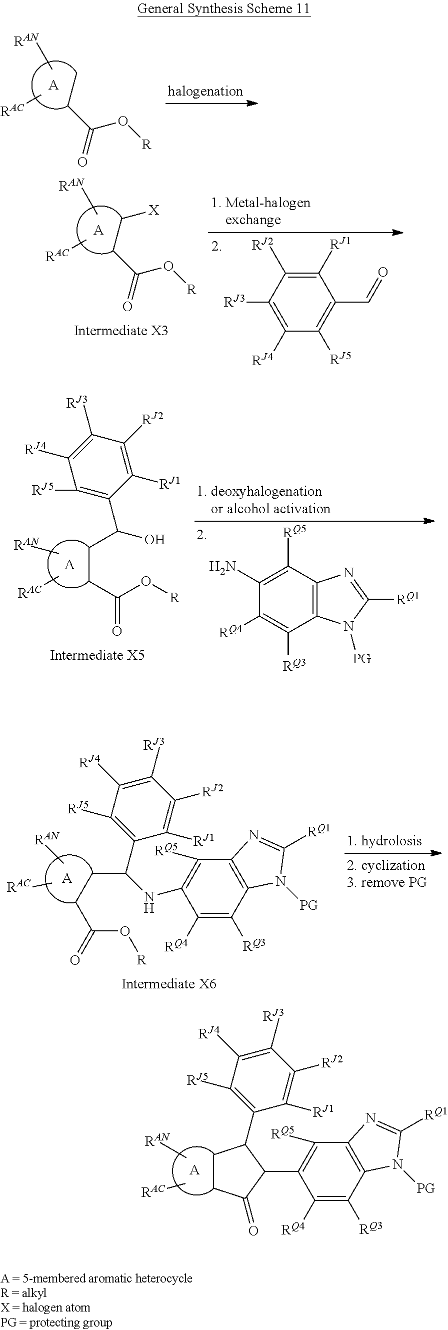

An additional example of such a method is illustrated in the following scheme.

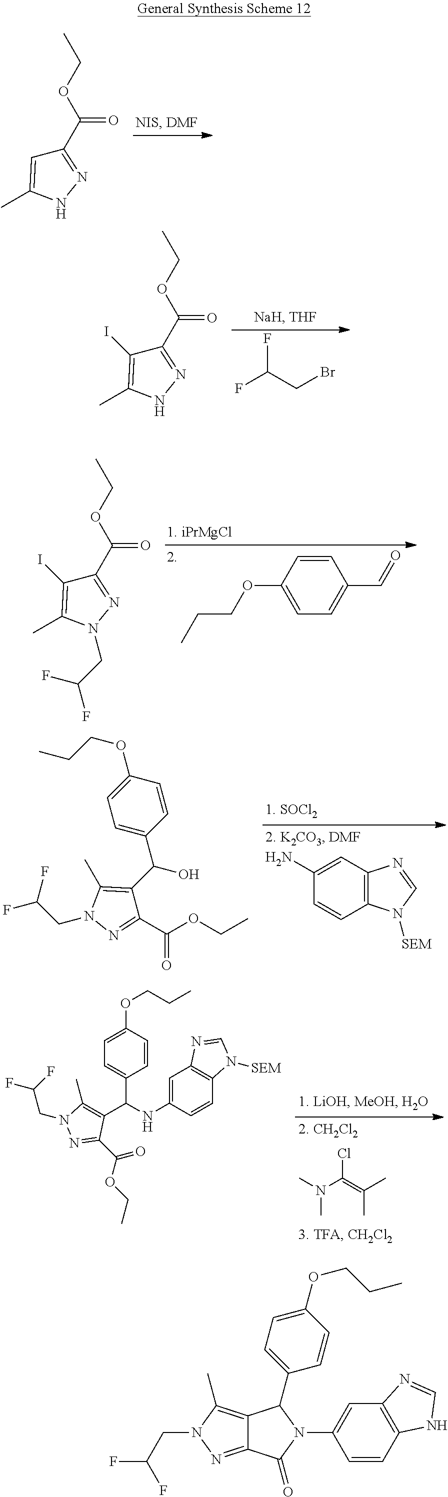

Alternatively, a substituted halogenated heterocyclic ester can be prepared by initial halogenation of a heterocyclic ester followed by further substitution.

For example, a pyrazole ester, can be halogenated with an atom such as bromine or iodine using standard electrophilic halogenation reagents such as NBS, NIS or I₂. Subsequent alkylation with a suitably substituted electrophilic alkane activated with a halogen atom or activated oxygen moiety, under basic conditions generates a pyrazole with up to four substituents, such as Intermediate X3.

An example of such a method is illustrated in the following scheme.

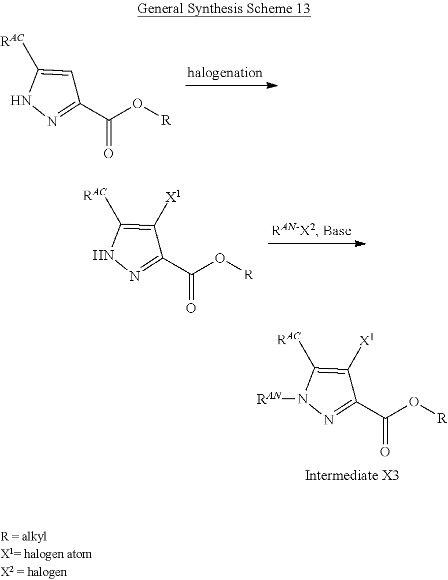

An intermediate alcohol, e.g., Intermediate X5, can also be generated by direct metalation of a substituted heterocycle and reaction with an aldehyde.

For example, a suitably substituted heterocyclic ester, can be deprotonated using an alkyl lithium, lithium amide, alkyl magnesium or similar base. Reaction of the resulting heterocyclic organometallic species with a suitably substituted aryl aldehyde, gives a secondary alcohol such as Intermediate X5.

An example of such a method is illustrated in the following scheme.

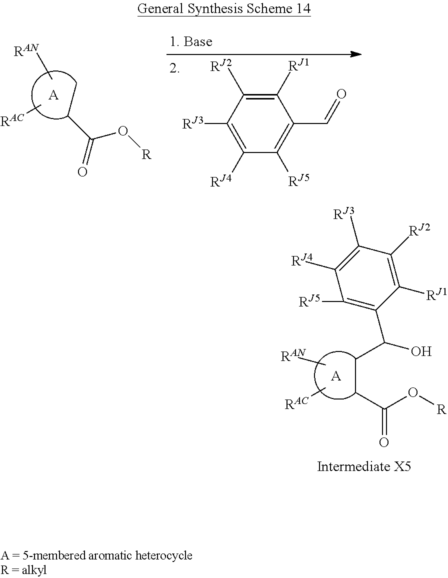

An additional example of such a method is illustrated in the following scheme.

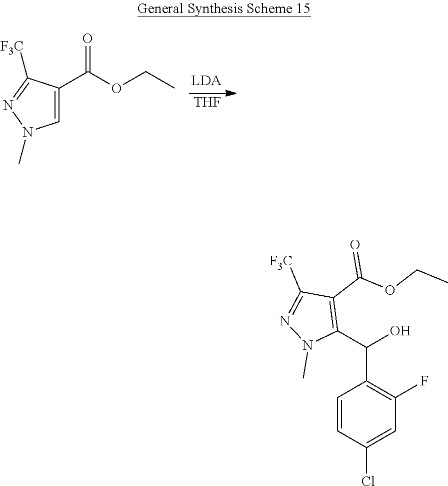 [SMILES: CCOC(=O)c1cn(C)nc1C(F)(F)F ; CCOC(=O)c1c(C(F)(F)F)nn(C)c1C(O)c1ccc(Cl)cc1F ; "THF"]

An intermediate secondary alcohol can also be prepared by addition of an aryl metallic reagent to a suitably substituted aldehyde.

For example, a hydroxymethyl substituted heterocycle can be oxidized to the suitably substituted aldehyde with an oxidizing agent such as MnO₂ or PCC. An aryl metallic reagent can be prepared from a suitably substituted aryl halide by reaction with an organometallic or metal reagent, such as iPrMgBr, nBuLi or Mg, and then added to the aldehyde to give the desired suitably substituted intermediate secondary alcohol, Intermediate X5.

An example of such a method is illustrated in the following scheme.

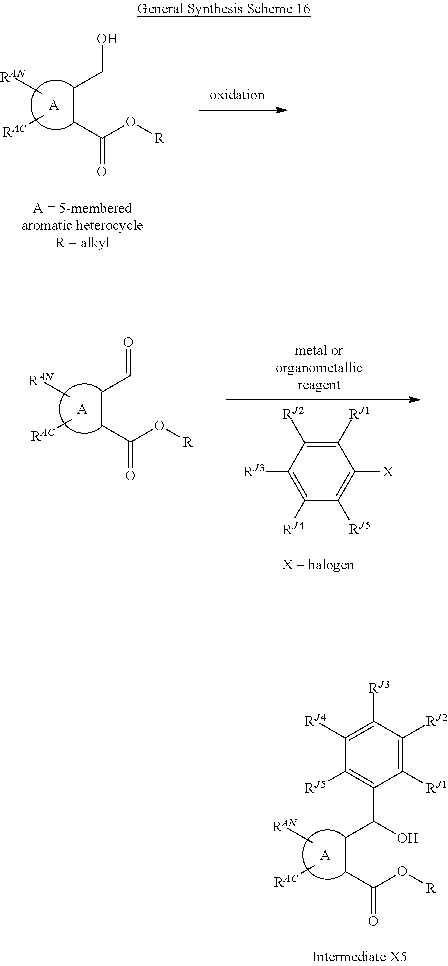

An additional example of such a method is illustrated in the following scheme.

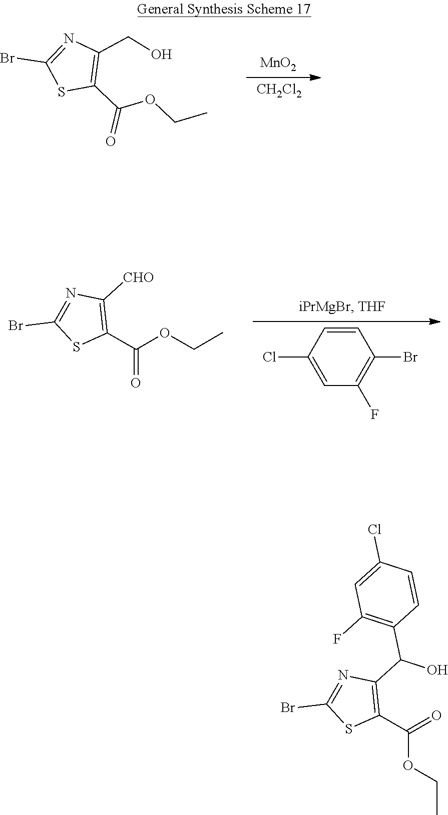

There are many suitable ways to prepare the substituted aryl aldehydes used as intermediates from diverse substrates. Some methods are reduction of a nitrile, oxidation of an aryl methyl group, formylation of an aromatic ring, and functionalization of an aryl aldehyde precursor.

For example, nucleophilic aromatic substitution of a suitably substituted fluoro benzonitrile, with a suitably substituted amine under basic conditions using a tertiary amine or other base, followed by reduction using DIBAL-H or other suitable reducing agent gives a substituted aryl aldehyde.

An example of such a method is illustrated in the following scheme.

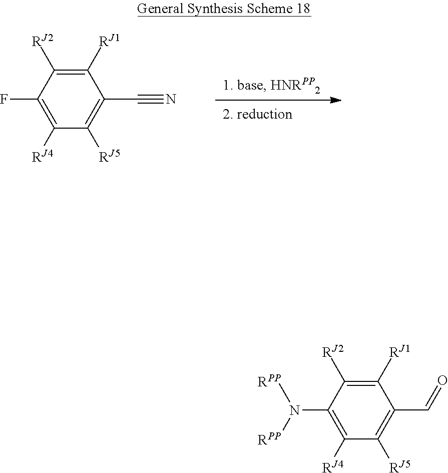

An additional example of such a method is illustrated in the following scheme.

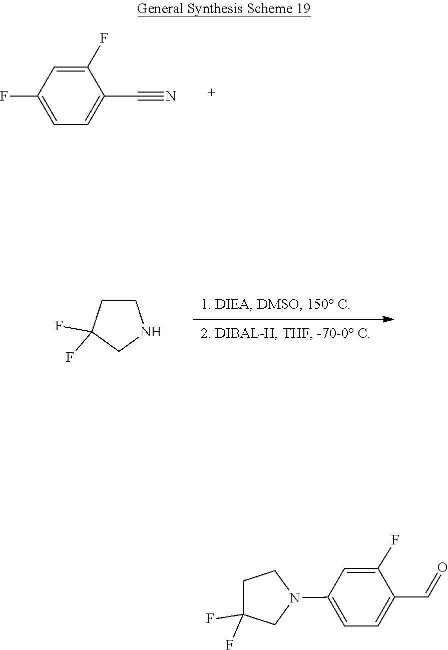 [SMILES: N#Cc1ccc(F)cc1F ; FC1(F)CCNC1 ; O=Cc1ccc(N2CCC(F)(F)C2)cc1F]

As an example, nucleophilic aromatic substitution of a suitably substituted fluoro benzaldehyde with a suitably substituted alcohol under basic conditions using K₂CO₃, an amine or other base, gives a substituted aryl aldehyde.

An example of such a method is illustrated in the following scheme.

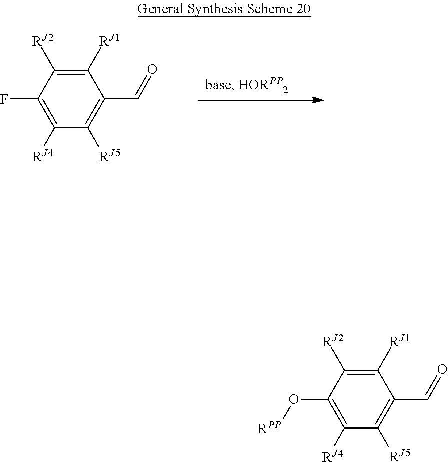

An additional example of such a method is illustrated in the following scheme.

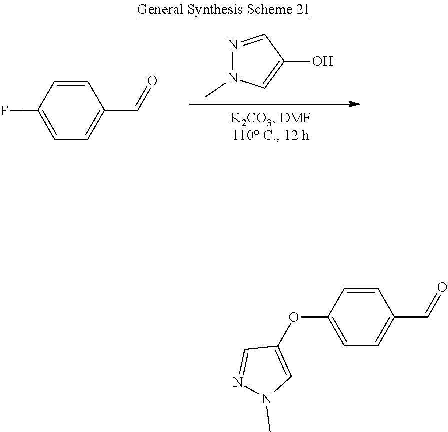

As another example, substitution of a suitably substituted halogenated benzaldehyde with a suitably substituted amine or amide using a transition metal catalyst and ligand, such as Pd₂dba₃/XPhos and basic conditions using Cs₂CO₃ or other base, gives a substituted aryl aldehyde. There are many transition metal, ligand and base combinations that can be used in an amination reaction of this type.

An example of such a method is illustrated in the following scheme.

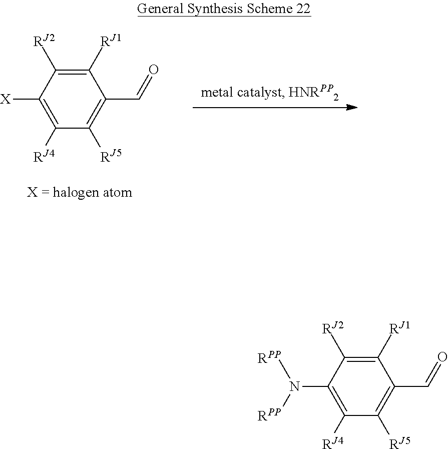

An additional example of such a method is illustrated in the following scheme.

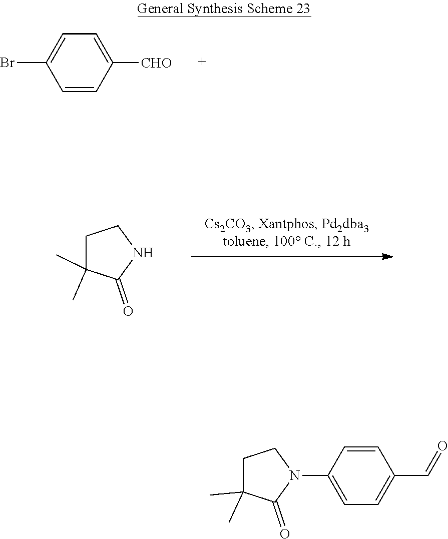

As another example, Suzuki reaction of a suitably substituted formyl-substituted aryl boronic acid or boronic ester with a suitably substituted aryl- or hetero-aryl halide using a transition metal catalyst and ligand, such as Pd(PPh₃)₄ and basic conditions using NaOH or other base, gives a substituted aryl aldehyde. There are many transition metal, ligand and base combinations that can be used in a Suzuki reaction of this type.

An example of such a method is illustrated in the following scheme.

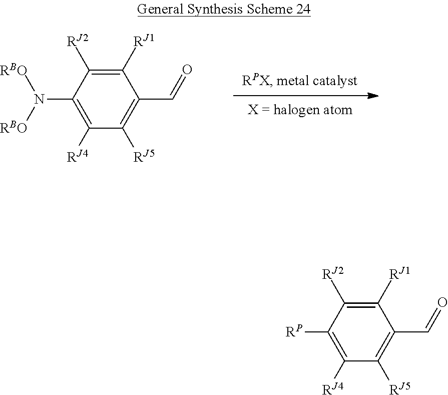

An additional example of such a method is illustrated in the following scheme.

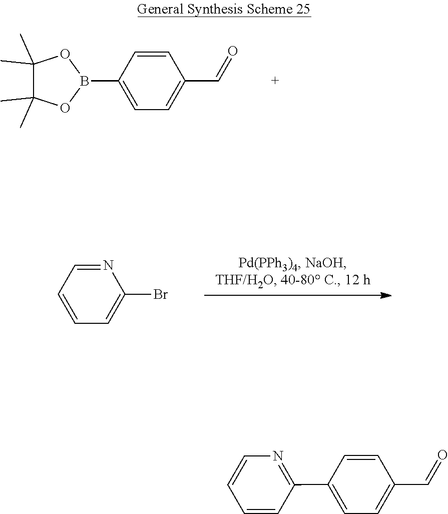 [SMILES: CC1(C)OB(c2ccc(C=O)cc2)OC1(C)C ; Brc1ccccn1 ; O=Cc1ccc(-c2ccccn2)cc1]

As another example, radical halogenation of a suitably substituted arylmethane with Br₂, NBS or similar reagent and a radical initiator such as benzoyl peroxide, followed by hydrolysis of the resulting benzyl bromide with an aqueous solution of Cs₂CO₃, or other base, gives a suitably substituted benzyl alcohol. Oxidation using a reagent such as MnO₂ gives the desired aryl aldehyde.

An example of such a method is illustrated in the following scheme.

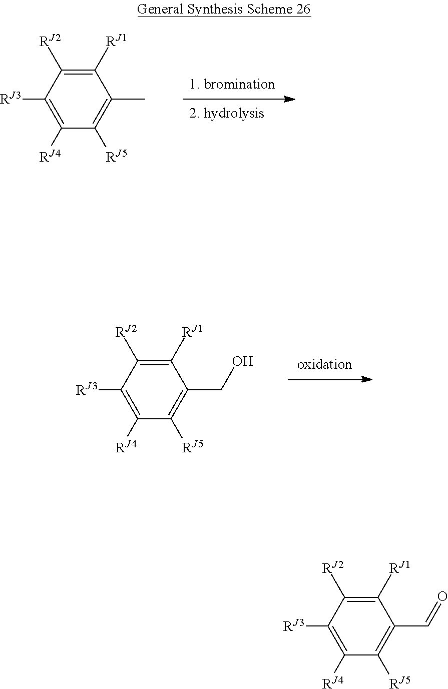

An additional example of such a method is illustrated in the following scheme.

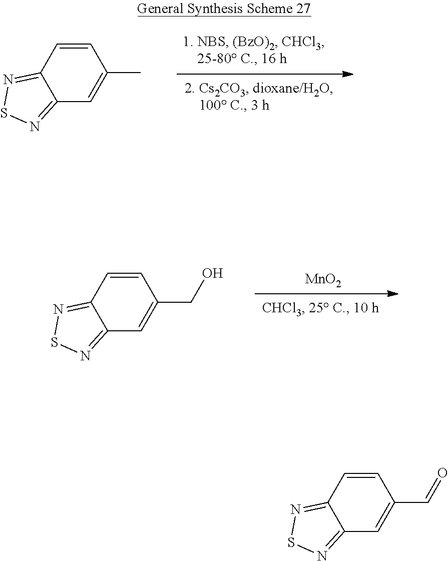

As another example, reaction between a suitably substituted electron-rich aromatic aldehyde and a formylating reagent, such as the Vilsmeier reagent generated in situ from POCl₃ and DMF, will furnish a suitably substituted aryl aldehyde.

An example of such a method is illustrated in the following scheme.

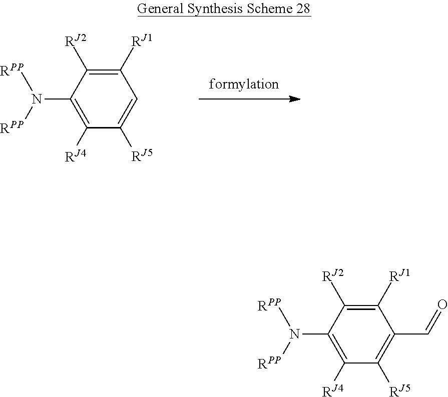

An additional example of such a method is illustrated in the following scheme.

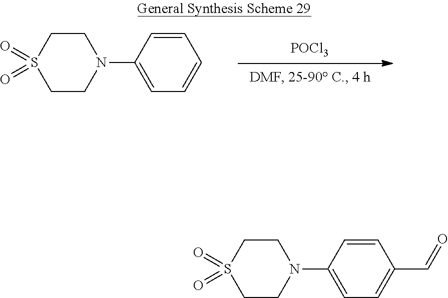 [SMILES: O=S1(=O)CCN(c2ccccc2)CC1 ; "POCl3" ; O=Cc1ccc(N2CCS(=O)(=O)CC2)cc1]

Another method for the synthesis of suitably substitute aryl aldehydes is via modification of a benzonitrile prior to reduction to the aldehyde.

For example a hydroxyl substituted benzonitrile can be alkylated with a suitably-substituted, electrophilic alkane activated with a halogen atom or activated oxygen moiety under basic conditions. When there are oxygen-containing functional groups present on the alkyl group, they can be converted to fluorine using a fluorinating reagent such as DAST. If desired, the oxygen-containing functional group can be oxidized further with IBX or another oxidizing agents, prior to fluorination. Reduction of the benzonitrile moiety with, for example, DIBAL-H, furnishes the suitably substituted aryl aldehyde.

An example of such a method is illustrated in the following scheme.

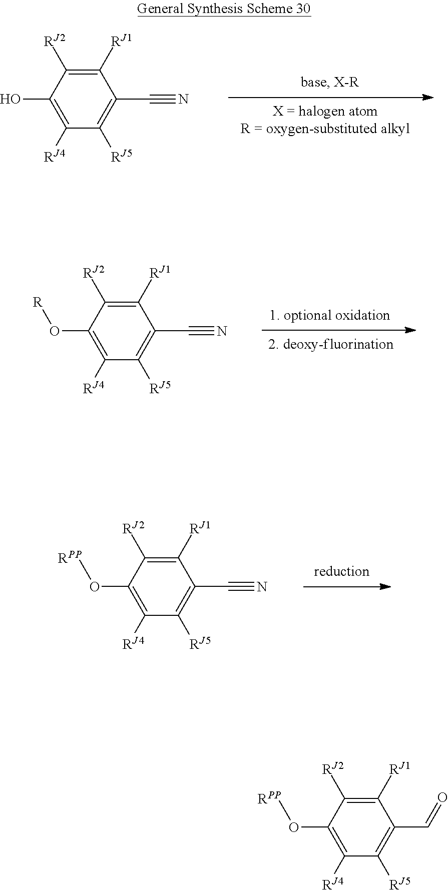

An additional example of such a method is illustrated in the following scheme.

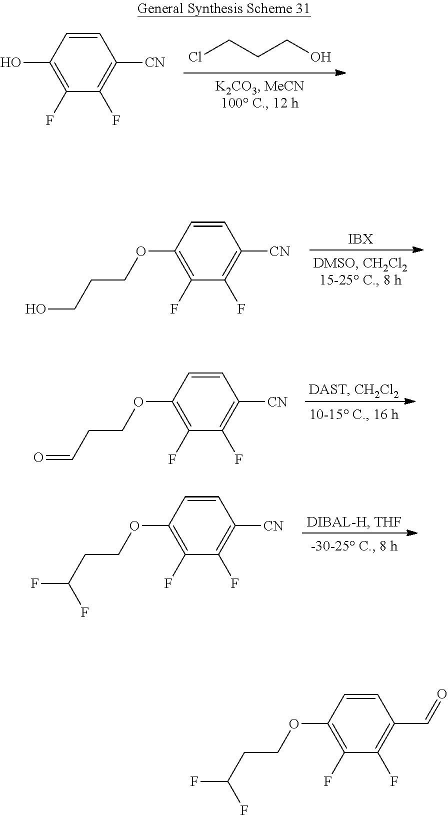

In another approach, certain compounds described herein may conveniently be prepared from a 4-(haloaryl)-4,5-dihydropyrrolo[3,4-c]pyrazol-6(2H)-one, e.g., Intermediate X4, via protection of any potentially reactive functionalities with a suitable protecting group, followed by displacement of the halo group with one of a number of possible reactions such as nucleophilic substitution or metal catalysed amination.

For example, a suitably substituted a 4-(haloaryl)-4,5-dihydropyrrolo[3,4-c]pyrazol-6(2H)-one is reacted with an activated protecting group (PG) such as SEMCl, under basic conditions. A halogen atom such as chlorine, bromine or iodine is displaced with an amine nucleophile using a transition metal catalyst and ligand, such as Pd₂dba₃/XPhos and a base, such as LiHMDS. There are many transition metal, ligand and base combinations that can be used in an amination reaction of this type. The protecting group is then removed, for example using TFA or TBAF for a SEM group.

An example of such a method is illustrated in the following scheme.

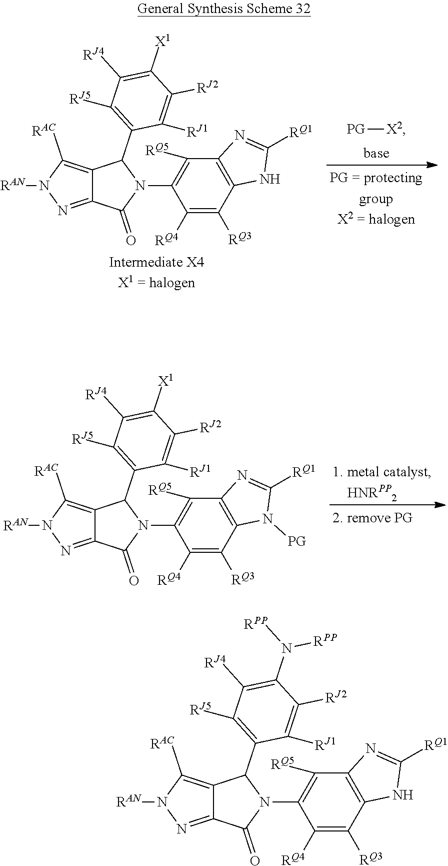

An additional example of such a method is illustrated in the following scheme.

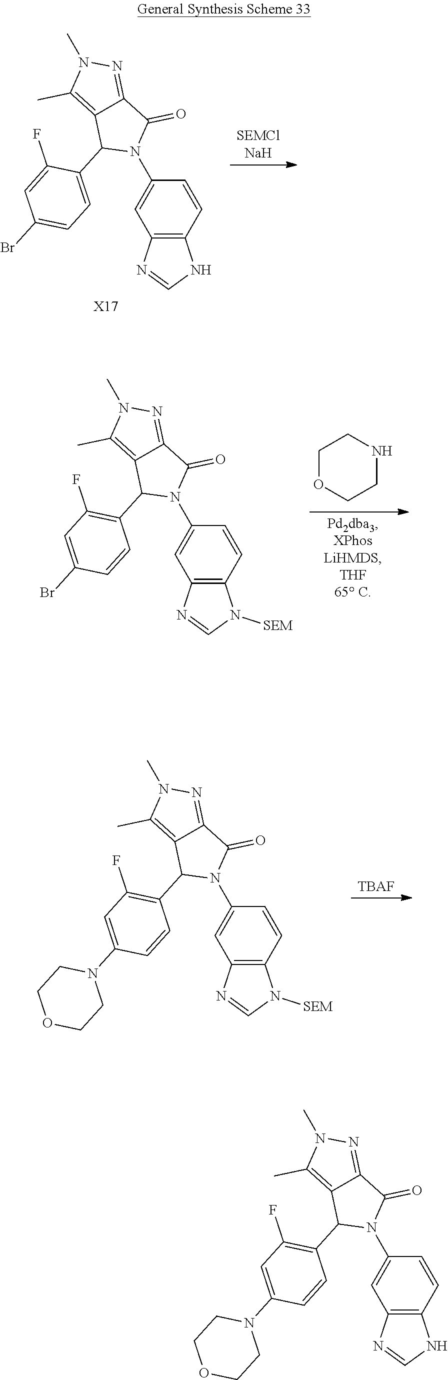

When a halogen atom is present in an intermediate it can be substituted with another group.

For an example in an intermediate 4,5-dihydropyrrolo[3,4-c]pyrazol-6(2H)-one substituted with a halogen X, such as Cl, Br or I the halogen can be reduced using a transition metal catalyst such as Pd/C and a hydrogen source, such as H₂ gas, formic acid or triethylsilane. Alternatively the halogen can be substituted in a Suzuki reaction using a boronic acid or ester, such as methylboronic acid, a Pd complex, such as Pd(PPh₃)₄ and base, such as K₂CO₃. There are many transition metal, ligand and base combinations that can be used in a Suzuki reaction of this type. In both cases, a protecting group, such as SEM, can be removed if necessary with a suitable reagent, such as TFA or TBAF.

Examples of such a method are illustrated in the following scheme.

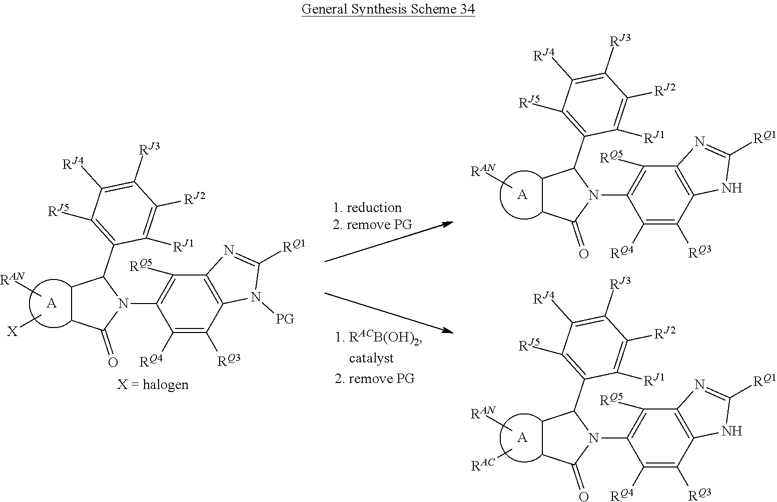

Additional examples of such a method are illustrated in the following schemes.

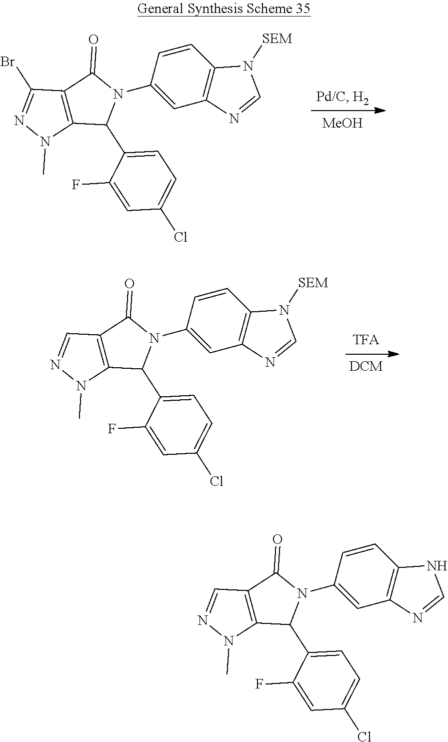

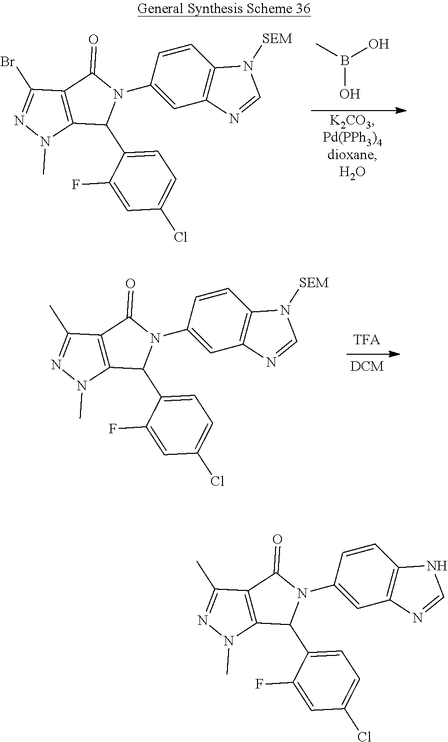

In another approach bicyclic heterocycles with a nitrogen atom at the ring junction, can be prepared by deprotonation at a carbon adjacent to a protected nitrogen in a suitably substituted heterocycle, followed by reaction with a suitably substituted aryl aldehyde. Activation of the resultant alcohol by conversion to a halide or other derivative, followed by displacement with a suitably protected and substituted amine gives an intermediate amine. Deprotection of the heterocyclic nitrogen and cyclization with an activated carbonic acid equivalent and deprotection gives a suitably substituted bicyclic imidazol-5-one.

For example, a protected heterocycle is deprotonated with a base such as an alkyl lithium or lithium amide and reacted with a substituted aldehyde. The resulting alcohol is converted to a leaving group with a reagent such as SOCl₂ or MsCl and base. Displacement with an amine gives an intermediate which is deprotected, reacted with an activated carbonic acid such as CDI or COCl₂ and further deprotected to give a suitably substituted, bicyclic imidazol-5-one.

An example of such a method is illustrated in the following scheme.

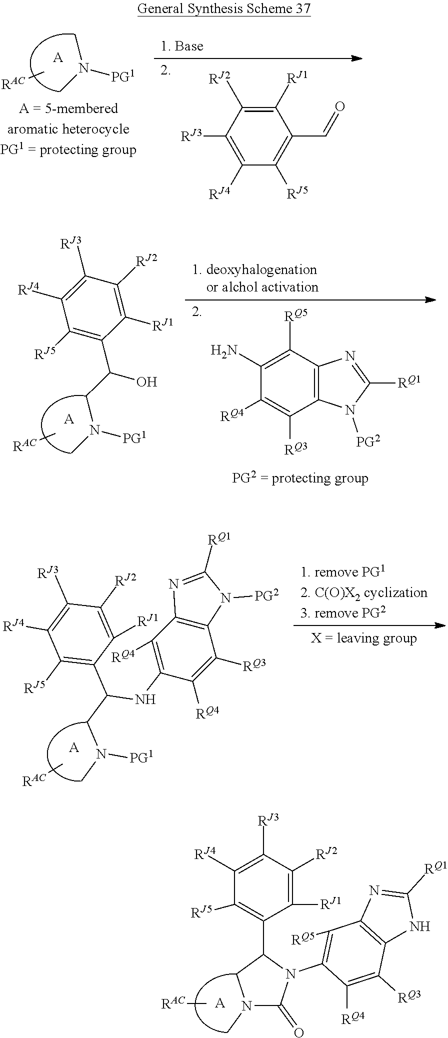

In another approach, certain compounds described herein may conveniently be prepared from a 4-(haloaryl)-4,5-dihydropyrrolo-fused heterocycle, e.g., Intermediate X4, via protection of any potentially reactive functionalities with a suitable protecting group, followed by displacement of the halo group with one of a number of possible reactions such a Suzuki reaction, or metal catalysed heterocycle coupling. Alternatively, the protected Intermediate X7 can be converted to a boronic ester via a Miyaura borylation, and then coupled to an aryl or heteroaryl bromide in a Suzuki reaction.

For example, a suitably substituted a 4-(haloaryl)-4,5-dihydropyrrolo[3,4-c]pyrazol-6(2H)-one is reacted with an activated protecting group (PG) such as SEMCl, under basic conditions. A halogen atom such as chlorine, bromine or iodine is coupled to a boronic ester using a transition metal catalyst and ligand, such as Pd(dppf)Cl₂ and a base, such as Na₂CO₃. There are many transition metal, ligand, and base combinations that can be used in an amination reaction of this type. The protecting group is then removed, for example using TFA or TBAF for a SEM group or may be removed concomitantly under the conditions of the coupling reaction.

Examples of such a method are illustrated in the following schemes.

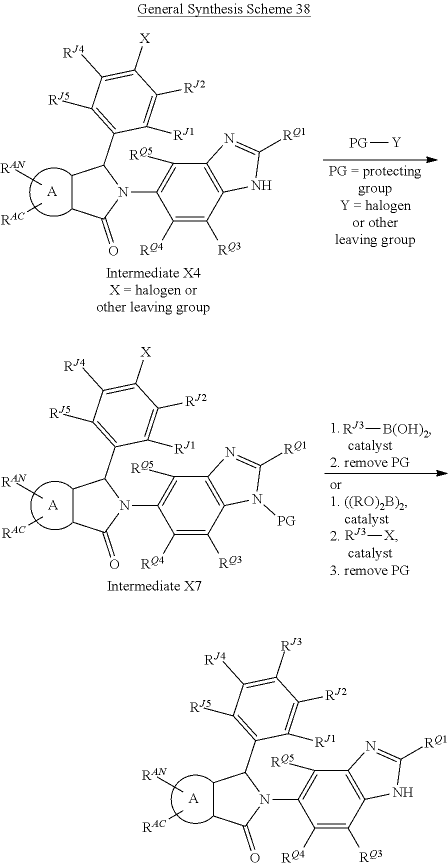

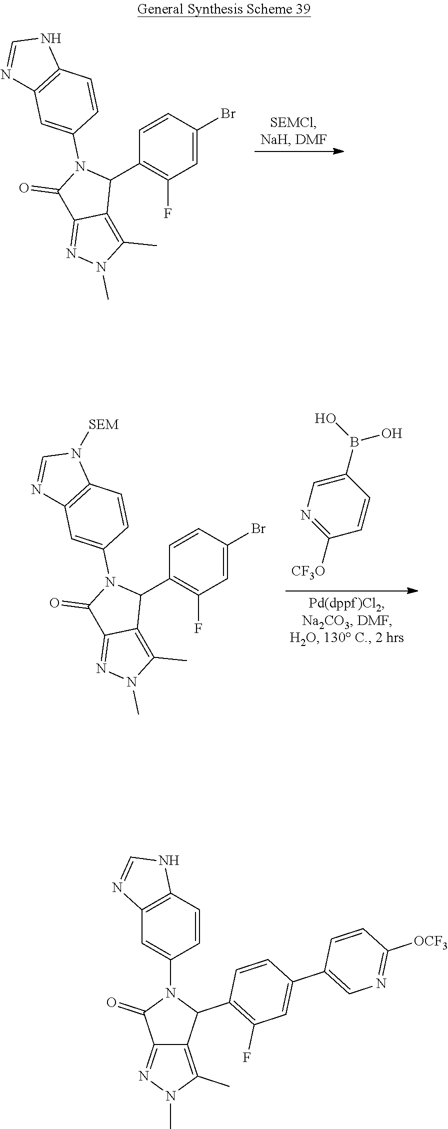

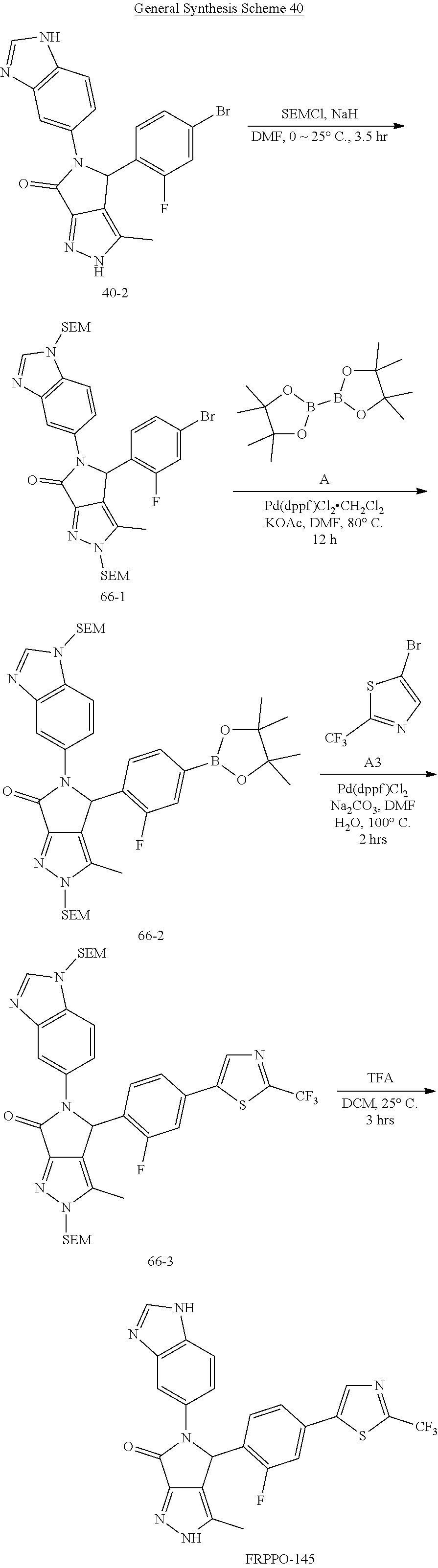

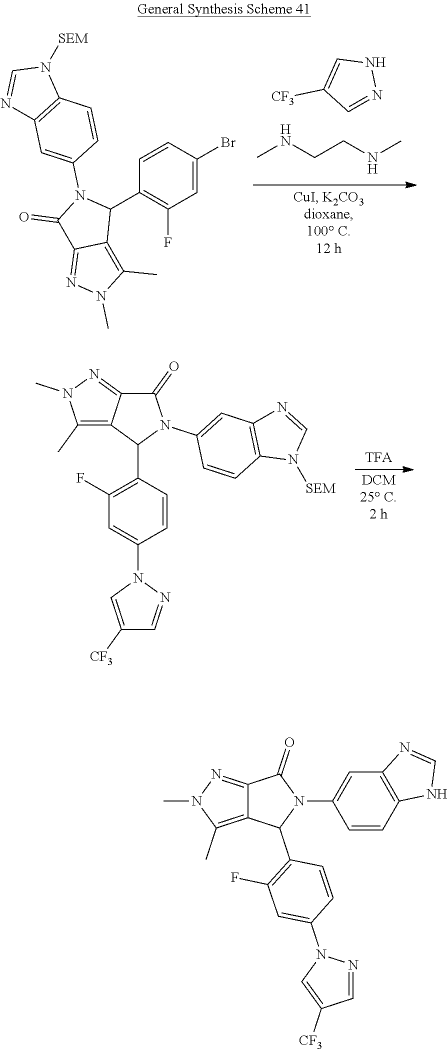

In another approach, certain hydroxy-substituted compounds described herein may conveniently be prepared by a method similar to that exemplified in General Synthesis Schemes 3 and 4 where a suitably substituted and protected 2,5-dihydroxy-4-oxopent-2-enoate ester Intermediate X8 is prepared by base catalysed condensation of a protected hydroxyacetone and an alkyl oxalate. For example, the hydroxy group can be protected with a benzyl group which is removed later via hydrogenation. Intermediate X8 can be condensed with a substituted benzaldehyde and aminoheterocycle to give Intermediate X9. Further condensation with a substituted hydrazine, substitution of a suitable halogen or other leaving group X and final deprotection will give the desired product. Alternatively, the Intermediate X9 can deprotected, oxidised to an aldehyde, reacted with an organic metallic reagent and deprotected to give the desired product.

Examples of such a method are illustrated in the following schemes.

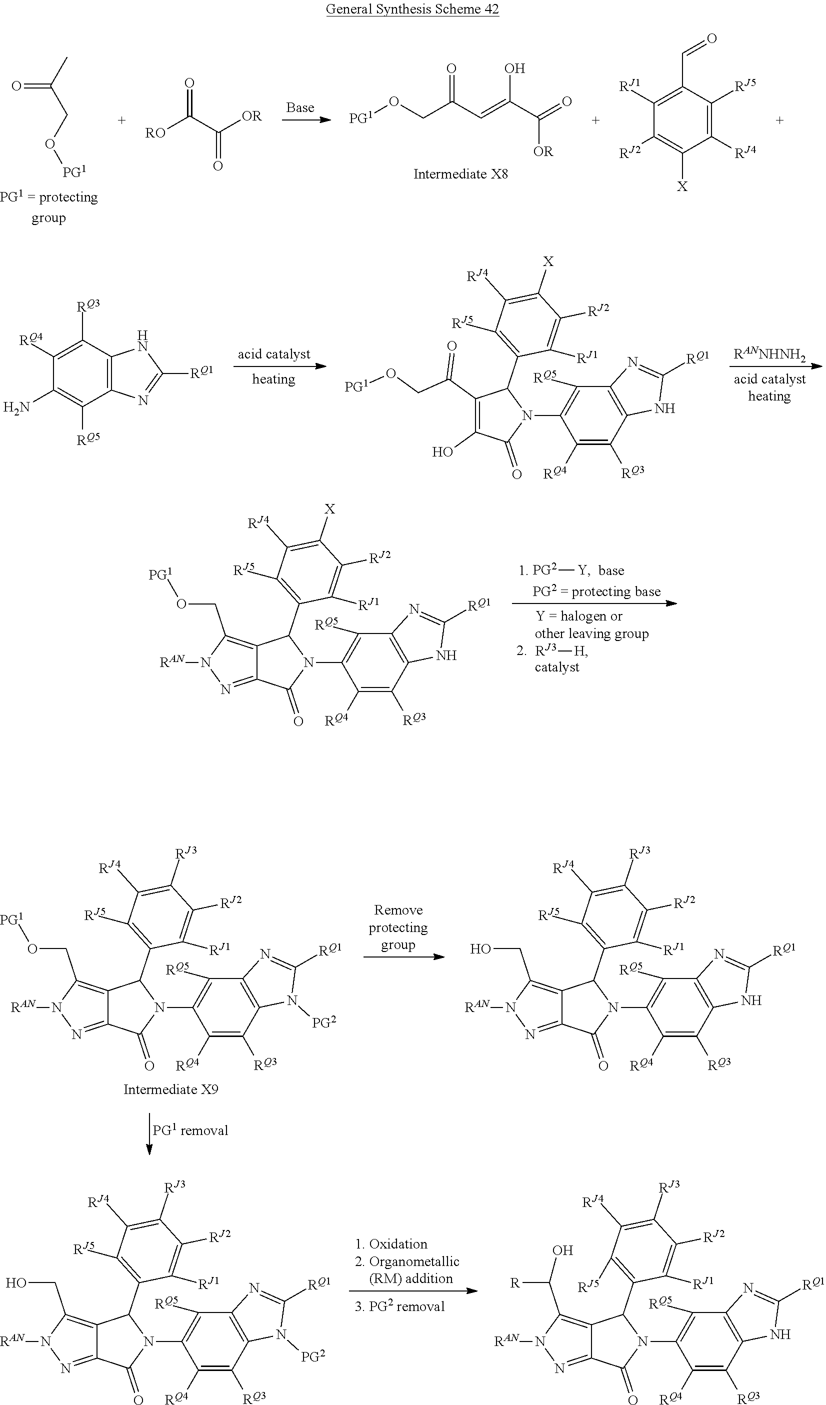

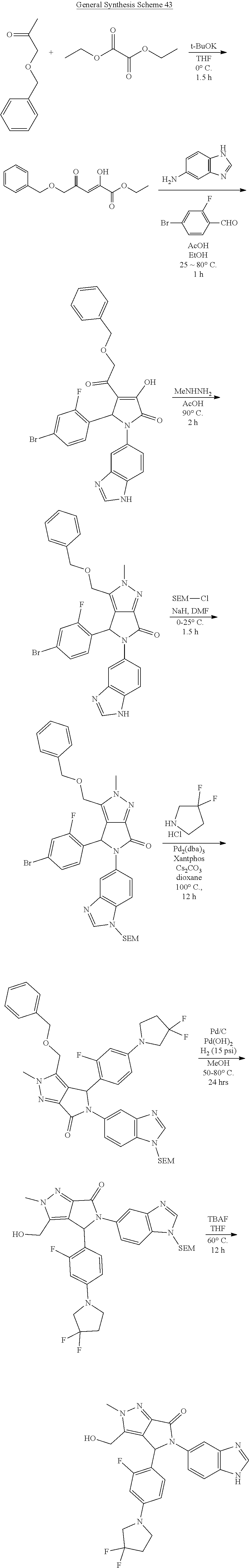

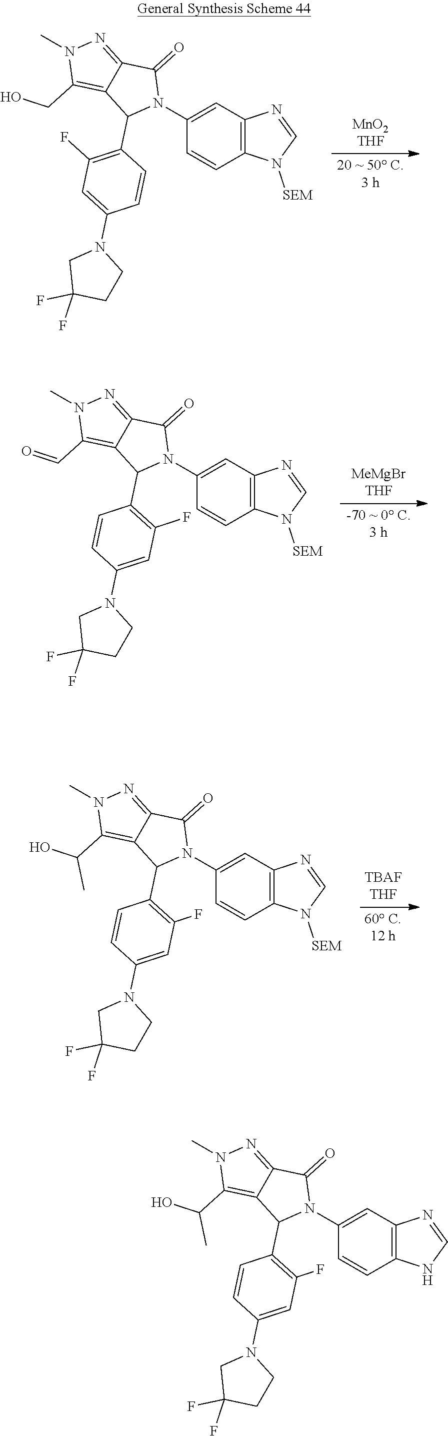 [SMILES: Cn1nc2c(c1CO)C(c1ccc(N3CCC(F)(F)C3)cc1F)N(c1ccc3c(c1)ncn3COCC[Si](C)(C)C)C2=O ; Cn1nc2c(c1C=O)C(c1ccc(N3CCC(F)(F)C3)cc1F)N(c1ccc3c(c1)ncn3COCC[Si](C)(C)C)C2=O ; CC(O)c1c2c(nn1C)C(=O)N(c1ccc3c(c1)ncn3COCC[Si](C)(C)C)C2c1ccc(N2CCC(F)(F)C2)cc1F ; CC(O)c1c2c(nn1C)C(=O)N(c1ccc3[nH]cnc3c1)C2c1ccc(N2CCC(F)(F)C2)cc1F]

In another approach, certain compounds described herein may conveniently be prepared by cyclization of a substituted 2,4-dioxobutanoic ester with a substituted benzaldehyde and an ammonia source such as ammonium acetate in the presence of an acid catalyst. The resulting 4-acyl-3-hydroxy-1,5-dihydro-2H-pyrrol-2-one (or tautomer) intermediate can be cyclized to a 4,5-dihydropyrrolo[3,4-c]pyrazol-6(2H)-one by treatment with a substituted hydrazine and an acid catalyst such a acetic acid. Further reaction with a leaving group (for example a halogen such as bromide) substituted heteroaromatic compound with a metal catalyst and ligand gives the desired final compound. There are many transition metal, ligand and base combinations that can be used in a substitution reaction of this type including CuI, N,N′-dimethylethylenediamine and K₃PO₄.

An example of such a method is illustrated in the following schemes.

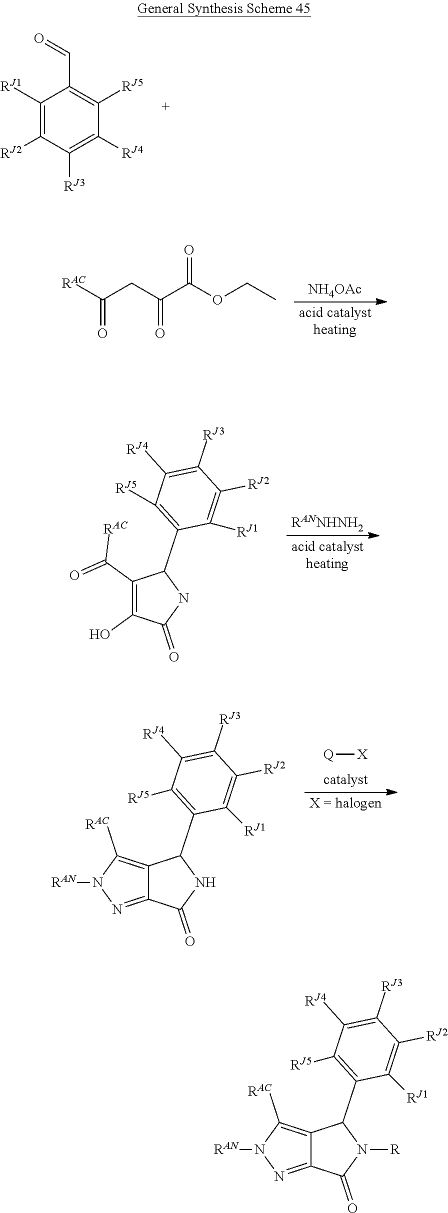

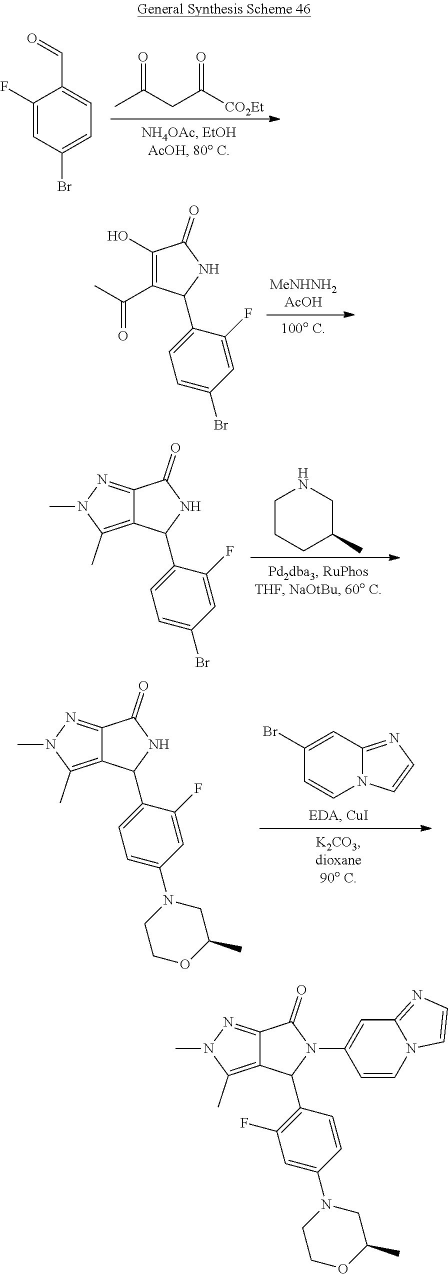

### Compositions

One aspect of the present invention pertains to a composition (e.g., a pharmaceutical composition) comprising a FRPPO compound, as described herein, and a pharmaceutically acceptable carrier, diluent, or excipient.

Another aspect of the present invention pertains to a method of preparing a composition (e.g., a pharmaceutical composition) comprising mixing a FRPPO compound, as described herein, and a pharmaceutically acceptable carrier, diluent, or excipient.

### Uses

The FRPPO compounds, as described herein, inhibit glutaminyl-peptide cyclotransferase-like (isoQC) enzyme and/or glutaminyl-peptide cyclotransferase (QC) enzyme (e.g., inhibit or reduce or block the activity or function of isoQC and/or QC enzyme).

In one embodiment, the FRPPO compounds inhibit glutaminyl-peptide cyclotransferase-like (isoQC) enzyme (e.g., inhibit or reduce or block the activity or function of isoQC enzyme).

In one embodiment, the FRPPO compounds inhibit glutaminyl-peptide cyclotransferase (QC) enzyme (e.g., inhibit or reduce or block the activity or function of QC enzyme).

The FRPPO compounds, as described herein, inhibit both glutaminyl-peptide cyclotransferase-like (isoQC) enzyme and glutaminyl-peptide cyclotransferase (QC) enzyme (e.g., inhibit or reduce or block the activity or function of isoQC and/or QC enzyme).

Accordingly, the FRPPO compounds, as described herein, are useful, for example, in the treatment of disorders (e.g., diseases) that are ameliorated by the inhibition of isoQC and/or QC enzyme (e.g., by the inhibition or reduction or blockage of the activity or function of isoQC and/or QC enzyme).

Use in Methods of Inhibiting isoQC and/or QC Enzyme

Another aspect of the present invention pertains to a method of inhibiting glutaminyl-peptide cyclotransferase-like (isoQC) enzyme and/or glutaminyl-peptide cyclotransferase (QC) enzyme (e.g., inhibiting or reducing or blocking the activity or function of isoQC and/or QC enzyme), in vitro or in vivo, comprising contacting the isoQC and/or QC enzyme with an effective amount of a FRPPO compound, as described herein. Another aspect of the present invention pertains to a method of inhibiting glutaminyl-peptide cyclotransferase-like (isoQC) enzyme and/or glutaminyl-peptide cyclotransferase (QC) enzyme (e.g., inhibiting or reducing or blocking the activity or function of isoQC and/or QC enzyme) in a cell, in vitro or in vivo, comprising contacting the cell with an effective amount of a FRPPO compound, as described herein.

In one embodiment, the method is performed in vitro.

In one embodiment, the method is performed in vivo.

In one embodiment, the FRPPO compound is provided in the form of a pharmaceutically acceptable composition.

One of ordinary skill in the art is readily able to determine whether or not a candidate compound inhibits isoQC and/or QC enzyme (e.g., inhibits or reduces or blocks or the activity or function of isoQC and/or QC enzyme). For example, suitable assays are described herein and/or are known in the art.

One of ordinary skill in the art is readily able to determine whether or not a candidate compound inhibits isoQC and/or QC enzyme (e.g., inhibits or reduces or blocks or the activity or function of isoQC and/or QC enzyme) in a cell. For example, a sample of cells may be grown in vitro and a compound brought into contact with said cells, and the effect of the compound on those cells observed. As an example of “effect,” the morphological status of the cells (e.g., alive or dead, etc.) may be determined. Where the compound is found to exert an influence on the cells, this may be used as a prognostic or diagnostic marker of the efficacy of the compound in methods of treating a patient carrying cells of the same cellular type.

### Use in Methods of Inhibiting Cell Proliferation, etc.

The FRPPO compounds described herein, may e.g., (a) regulate (e.g., inhibit) cell proliferation; (b) inhibit cell cycle progression; (c) promote apoptosis; or (d) a combination of one or more of these.

Another aspect of the present invention pertains to a method of regulating (e.g., inhibiting) cell proliferation (e.g., proliferation of a cell), inhibiting cell cycle progression, promoting apoptosis, or a combination of one or more these, in vitro or in vivo, comprising contacting a cell with an effective amount of a FRPPO compound, as described herein.

The FRPPO compounds described herein may be used in methods to reduce the formation of a pyroglutamyl residue at the N-terminus of CD47 expressed at the surface of a first cell in the subject. This can result in a reduction or inhibition of the binding or other interaction between the CD47 on the surface of the first cell, and SIRPα on the surface of a second cell.

The FRPPO compounds described herein may be used in methods to reduce binding or other interaction between CD47 on the surface of a first cell and SIRPα on the surface of a second cell.

The first cell with CD47 on the surface may be a cell which is a diseased cell, or other undesirable cell. The cell may be selected from: a cancer cell expressing or overexpressing CD47, a vascular smooth muscle cells expressing or overexpressing CD47, a diseased endothelial cell expressing or overexpressing CD47, a diseased cell infected by a pathogen, which is optionally a virus, expressing or overexpressing CD47, and a diseased cell undergoing fibrosis expressing or overexpressing CD47 on its cell surface.

The first cell may overexpress CD47 on its surface (i.e., it is upregulated).

The second cell expressing the SIRPα on its surface may be an immune cell such as a phagocyte cell (e.g., macrophage, neutrophil). Optionally the second cell is a myeloid cell, which is optionally selected from the group consisting of a macrophage, monocyte, neutrophil, basophil, eosinophil, and dendritic cell.

As explained above, reducing binding or other interaction may be achieved by inhibiting QPCTL in the first cell, and thereby inhibiting pyroglutamylation of the N-terminal glutamine moiety of CD47 on the surface of the first cell.

Reducing binding between said CD47 on the surface of said first cell and said SIRPα on the surface of said second cell may target said first cell with CD47 on the surface for phagocytosis, antibody-dependent cellular cytotoxicity (ADCC) or antibody-dependent cellular phagocytosis (abbreviated ADCP). In particular, the recognition of tumor-specific antigens (or other antigens associated with diseased or undesirable cells) by therapeutic antibodies can result in the coating, or opsonization, of the cells, and this can lead to ADCP.

The FRPPO compounds described herein may be used in methods to promote immunotherapy.

The FRPPO compounds described herein may be used in methods to promote immune-cell mediated killing of diseased or other undesirable cells expressing, or upregulating CD47.

The diseased or other undesirable cells may be those discussed above.

The immune cells may be those discussed above.

In one embodiment, the method is performed in vitro.

In one embodiment, the method is performed in vivo.

In one embodiment, the FRPPO compound is provided in the form of a pharmaceutically acceptable composition.

Any type of cell may be treated or targeted, including but not limited to, blood (including, e.g., neutrophils, eosinophils, basophils, lymphocytes, monocytes, erythrocytes, thrombocytes), lung, gastrointestinal (including, e.g., bowel, colon), breast (mammary), ovarian, prostate, liver (hepatic), kidney (renal), bladder, pancreas, brain, and skin cells.

One of ordinary skill in the art is readily able to determine whether or not a candidate compound regulates (e.g., inhibits) cell proliferation, etc. For example, assays which may conveniently be used to assess the activity offered by a particular compound are described herein and/or are known in the art.

In the light of the disclosure herein, one of ordinary skill in the art is readily able to confirm that a candidate compound inhibits formation of a pyroglutamyl residue at the N-terminus of CD47 and/or inhibits binding between said CD47 on the surface of said first cell and said SIRPα on the surface of said second cell. For example, assays which may conveniently be used to assess or confirm the activity offered by a particular compound are described herein and/or are known in the art. Assays may be performed in vitro e.g. using purified enzymes. Alternatively assays may be cell-based. For example they may assess whether the compound reduces the formation of a pyroglutamyl residue at the N-terminus of CD47 expressed at the surface of a first cell, with a resultant reduction or inhibition of the binding or other interaction between the CD47 on the surface of that first cell, and SIRPα on the surface of a second cell.

Alternatively, for example, a sample of cells (e.g., from a tumour) may be grown in vitro and a compound brought into contact with said cells, and the effect of the compound on those cells observed. As an example of “effect,” the morphological status of the cells (e.g., alive or dead, etc.) may be determined. Where the compound is found to exert an influence on the cells, this may be used as a prognostic or diagnostic marker of the efficacy of the compound in methods of treating a patient carrying cells of the same cellular type.

### Use in Methods of Therapy

Another aspect of the present invention pertains to a FRPPO compound as described herein for use in a method of treatment of the human or animal body by therapy, for example, for use in a method of treatment of a disorder (e.g., a disease) as described herein.

Another aspect of the present invention pertains to use of a FRPPO compound as described herein in a method of treatment of the human or animal body by therapy, for example, in a method of treatment of a disorder (e.g., a disease) as described herein.

### Use in the Manufacture of Medicaments

Another aspect of the present invention pertains to use of a FRPPO compound, as described herein, in the manufacture of a medicament, for example, for use in a method of treatment, for example, for use in a method of treatment of a disorder (e.g., a disease) as described herein.

In one embodiment, the medicament comprises the FRPPO compound.

### Methods of Treatment

Another aspect of the present invention pertains to a method of treatment, for example, a method of treatment of a disorder (e.g., a disease) as described herein, comprising administering to a subject in need of treatment a therapeutically-effective amount of a FRPPO compound, as described herein, preferably in the form of a pharmaceutical composition.

Disorders Treated—Disorders Ameliorated by the Inhibition of isoQC and/or QC Enzyme

In one embodiment (e.g., of compounds for use in methods of therapy, of use in methods of therapy, of use in the manufacture of medicaments, of methods of treatment), the treatment is treatment of a disorder (e.g., a disease) that is ameliorated by the inhibition of isoQC and/or QC enzyme (e.g., by the inhibition or reduction or blockage of the activity or function of isoQC and/or QC enzyme).

### Disorders Treated

Disorders which may be treated by the FRPPO compounds as described herein include those the aetiology of which involve or requires the CD47-SIRPα signalling axis. Such diseases include those in which diseased cells, or other undesirable cells, evade immune surveillance by expression or over-expression of CD47. Such diseases may thus be treated by reducing pyroglutamylation of CD47 in such cells, resulting in reduced binding between CD47 on the surface of such cells and SIRPα on the surface of a second cell, such as an immune cell.

In one embodiment (e.g., of compounds for use in methods of therapy, of use in methods of therapy, of use in the manufacture of medicaments, of methods of treatment), the treatment is treatment of a disorder, for example, cancer, atherosclerosis, fibrotic diseases, infectious diseases, Alzheimer's disease etc., as described herein.

### Proliferative Disorders

In one embodiment, the disorder is: a proliferative disorder.

The term “proliferative disorder,” as used herein, pertains to an unwanted or uncontrolled cellular proliferation of excessive or abnormal cells which is undesired, such as neoplastic or hyperplastic growth.

In one embodiment, the proliferative disorder is characterised by benign, pre-malignant, malignant, pre-metastatic, metastatic, or non-metastatic cellular proliferation, including for example: neoplasms, hyperplasias, and tumours (e.g., histocytoma, glioma, astrocyoma, osteoma), cancers (see below), psoriasis, bone diseases, fibroproliferative disorders (e.g., of connective tissues), pulmonary fibrosis, atherosclerosis, smooth muscle cell proliferation in the blood vessels, such as stenosis or restenosis following angioplasty.

### Cancer

In one embodiment, the disorder is: cancer.

In one embodiment, the disorder is: leukemia, acute myeloid leukemia (AML), acute promyelocytic leukemia (APL), chronic myeloid leukemia (CML), acute lymphoblastic leukemia (ALL), T-cell acute lymphoblastic leukemia (T-ALL), lymphoma, B-cell lymphoma, T-cell lymphoma, Hodgkin's disease, non-Hodgkin's lymphoma (NHL), hairy cell lymphoma, Burkett's lymphoma, multiple myeloma (MM), myelodysplastic syndrome, lung cancer, adenocarcinoma, small cell lung cancer (SCLC), non-small cell lung cancer (NSCLC), mediastinum cancer, peritoneal cancer, mesothelioma, gastrointestinal cancer, gastric cancer, stomach cancer, bowel cancer, small bowel cancer, large bowel cancer, colon cancer, colon adenocarcinoma, colon adenoma, rectal cancer, colorectal cancer, leiomyosarcoma, breast cancer, gynaecological cancer, genito-urinary cancer, ovarian cancer, endometrial cancer, cervical cancer, prostate cancer, testicular cancer, seminoma, teratocarcinoma, liver cancer, kidney cancer, bladder cancer, urothelial cancer, biliary tract cancer, pancreatic cancer, exocrine pancreatic carcinoma, esophageal cancer, nasopharyngeal cancer, head and neck squamous cell carcinoma (HNSCC), skin cancer, squamous cancer, squamous cell carcinoma, Kaposi's sarcoma, melanoma, malignant melanoma, xeroderma pigmentosum, keratoacanthoma, bone cancer, bone sarcoma, osteosarcoma, rhabdomyosarcoma, fibrosarcoma, thyroid gland cancer, thyroid follicular cancer, adrenal gland cancer, nervous system cancer, brain cancer, astrocytoma, neuroblastoma, glioma, schwannoma, glioblastoma, or sarcoma.

In one embodiment, the disorder is: leukemia, acute myeloid leukemia (AML), acute promyelocytic leukemia (APL), chronic myeloid leukemia (CML), acute lymphoblastic leukemia (ALL), T cell acute lymphoblastic leukemia (T ALL), lymphoma, B-cell lymphoma, T-cell lymphoma, Hodgkin's disease, non-Hodgkin's lymphoma (NHL), hairy cell lymphoma, Burkett's lymphoma, multiple myeloma (MM), myelodysplastic syndrome, lung cancer, adenocarcinoma, small cell lung cancer (SCLC), non-small cell lung cancer (NSCLC), gastrointestinal cancer, gastric cancer, stomach cancer, bowel cancer, small bowel cancer, large bowel cancer, colon cancer, colon adenocarcinoma, colon adenoma, rectal cancer, colorectal cancer, breast cancer, gynaecological cancer, ovarian cancer, prostate cancer, bladder cancer, pancreatic cancer, exocrine pancreatic carcinoma, esophageal cancer, head and neck squamous cell carcinoma (HNSCC), skin cancer, squamous cancer, squamous cell carcinoma, Kaposi's sarcoma, melanoma, malignant melanoma, xenoderoma pigmentoum, osteosarcoma, nervous system cancer, brain cancer, astrocytoma, neuroblastoma, glioma, schwannoma, glioblastoma, or sarcoma.

In one embodiment, the disorder is: gastrointestinal cancer, gastric cancer, stomach cancer, esophageal cancer, head and neck squamous cell carcinoma (HNSCC), non-small cell lung cancer (NSCLC), breast cancer, colorectal cancer, bowel cancer, large bowel cancer, colon cancer, colon adenocarcinoma, colon adenoma, rectal cancer, ovarian cancer, pancreatic cancer, exocrine pancreatic carcinoma, leukemia, acute myeloid leukemia (AML), myelodysplastic syndrome, lymphoma, B-cell lymphoma, non-Hodgkin's lymphoma (NHL), urothelial cancer, or peritoneal cancer.

### Atherosclerosis

In one embodiment, the disorder is: atherosclerosis.

The term “atherosclerosis” as used herein refers to condition recognized as the main disease process underlying heart attack and stroke. More specifically, atherosclerosis is characterized as a systemic, progressive disease process in which the arterial wall thickens through a pathological process involving inflammation, oxidative stress, and dyslipidemia. This pathological process leads to plaque formation and flow limitation in the vessel lumen of subjects afflicted with the condition.

The mechanisms underlying atherosclerosis are being actively studied. For example, it has been reported that the accumulation of diseased vascular cells (e.g., diseased vascular smooth muscle cells), diseased endothelial cells, and apoptotic cellular debris in the vessel lumen debris contributes to worsen the pathological process leading to plaque formation. A recent study has revealed that diseased cells (such as diseased vascular smooth muscle cells and diseased endothelial cells) upregulate the expression of CD47 at their cell surface thereby conveying a “don't eat me signal”, which allows said diseased cells to evade phagocytosis by phagocyte cells, e.g., macrophages, so that diseased cells are not cleared by the immune system (see, e.g., Kojima et al., 2016). This is consistent with the observation that CD47 is consistently upregulated in human atherosclerotic plaque compared to non-atherosclerotic vascular tissue, and in subjects with symptomatic cerebrovascular disease (stroke or transient ischemic attack) as compared to those with stable asymptomatic lesions (see, e.g., Kojima et al., 2016). It was further reported that inhibiting the CD47-SIRPα axis by administration of an anti-CD47 antibody improved clearance of diseased cells by phagocyte cells and ameliorated atherosclerosis (see, e.g., Kojima et al., 2016). Accordingly, isoQC and/or QC inhibitors, such as those described herein, may be useful therapeutic agents for the treatment of atherosclerosis.

### Fibrotic Diseases

In one embodiment, the disorder is: a fibrotic disease.

In one embodiment, the disorder is: scleroderma, idiopathic pulmonary fibrosis, liver cirrhosis, kidney fibrosis, lung fibrosis, bladder fibrosis, heart fibrosis, pancreas fibrosis, or myelofibrosis.

The term “fibrotic disease” as used herein refers to a condition that is characterized by the accumulation of excess extracellular matrix components (e.g., collagen, fibronectin) that forms fibrous connective tissue in and around an inflamed or damaged tissue. Fibrosis may cause overgrowth, hardening, and/or scarring that disrupts the architecture of the underlying organ or tissue. While controlled tissue remodeling and scarring is part of the normal wound healing process promoted by transdifferentiation of fibroblasts into myofibroblasts, excessive and persistent scarring due to severe or repetitive injury or dysregulated wound healing (e.g., persistence of myofibroblasts) can eventually result in permanent scarring, organ dysfunction and failure, and even death.

Fibrotic changes can occur in vascular disorders (e.g., peripheral vascular disease, cardiac disease, cerebral disease, etc.) and in all main tissue and organ systems (e.g., lung, liver, kidney, heart, skin, pancreas). Fibrotic disorders include a wide range of clinical presentations, including multisystemic disorders, such as systemic sclerosis, multifocal fibrosclerosis, scleroderma, myelofibrosis, and organ-specific disorders, such as pulmonary (e.g., idiopathic pulmonary fibrosis (IPF)), liver fibrosis, kidney fibrosis, pancreas fibrosis, heart fibrosis, and bladder fibrosis (see, e.g., Rosenbloom et al., 2010; Wynn et al., 2004; Wernig et al., 2017). The mechanisms underlying fibrotic diseases are being actively studied. For example, it has been reported that diseased cells such as diseased fibroblasts upregulate the expression of CD47 at their cell surface thereby conveying a “don't eat me signal”, which allows the diseased cells to evade phagocytosis by phagocyte cells, e.g., macrophages and/or neutrophils, so that diseased cells are not cleared by the immune system (see, e.g., Wernig et al., 2017). It was further found that inhibiting the CD47-SIRPα axis by treatment with an anti-CD47 antibody led to an increased phagocytosed diseased fibroblast, which in turn reduced fibrosis in the tissue (see, e.g., Wernig et al., 2017). Accordingly, isoQC and/or QC inhibitors, such as those described herein, may be useful therapeutic agents for the treatment of fibrotic diseases.

### Infectious Diseases

In one embodiment, the disorder is: an infectious disease (e.g., an infection).

In one embodiment, the disorder is: an infectious disease caused by a virus, bacterium, or protozoan.

In one embodiment, the disorder is: an infectious disease caused by a virus.

In one embodiment, the disorder is: an infectious disease caused by a bacterium.

In one embodiment, the disorder is: an infectious disease caused by a protozoan.

In one embodiment, the disorder is: an infectious disease caused by a pathogen selected from: a lentivirus, human T-lymphotropic virus (HTLV), an hepadna virus, hepatitis B virus, a herpes virus, human papilloma virus, la crosse virus, Yersinia sp., Yersinia pestis, Yersinia pseudotuberculosis, Yersinia enterocolitica, Franciscella sp., Helicobacter sp., Helicobacter pylori, Pasteurella sp., Vibrio sp., Vibrio cholerae, Vibrio parahemolyticus, Legionella sp., Legionella pneumophila, Listeria sp., Listeria monocytogenes, Mycoplasma sp., Mycoplasma hominis, Mycoplasma pneumoniae, Mycobacterium sp., Mycobacterium tuberculosis, Mycobacterium leprae, Rickettsia sp., Rickettsia rickettsii, Rickettsia typhi, a Plasmodium, a Trypanosoma, a Giardia, a Toxoplasma, and a Leishmania.

Since the physiological function of the SIRPa-CD47 axis is thought to allow the immune system to discriminate self from non-self, inhibition of isoQC and/or QC in cells that, due to an infection, possess a pro-phagocytic signal may help to combat the infection. See, e.g., van den Berg et al., 2008. Accordingly, isoQC and/or QC inhibitors, such as those described herein, may be useful therapeutic agents for the treatment of infectious diseases (e.g., infections).

### Alzheimer's Disease (AD)

In one embodiment, the disorder is: Alzheimer's disease.

Schilling et al., 2008, demonstrates that pyroglutamylation of the Abeta protein is important in the pathogenesis of Alzheimer's disease. Accordingly, isoQC and/or QC inhibitors, such as those described herein, may be useful therapeutic agents for the treatment of Alzheimer's disease.

### Non-Alcoholic Steatohepatitis (NASH)

In one embodiment, the disorder is: non-alcoholic steatohepatitis (NASH).

Cynis et al., 2013, describes the potential application of pyroglutamylation inhibitors for the treatment of non-alcoholic steatohepatitis (NASH) through inhibition of CCL2 mediated inflammation. Accordingly, isoQC and/or QC inhibitors, such as those described herein, may be useful therapeutic agents for the treatment of NASH.

### Septic Arthritis

In one embodiment, the disorder is: septic arthritis.

Hellvard et al., 2012, describe the use of glutaminyl cyclase inhibitors for the treatment of septic arthritis. Accordingly, isoQC and/or QC inhibitors, such as those described herein, may be useful therapeutic agents for the treatment of septic arthritis.

### COPD/Asthma/Allergies

In one embodiment, the disorder is: chronic obstructive pulmonary disease (COPD), asthma, or an allergy.

In one embodiment, the disorder is: chronic obstructive pulmonary disease (COPD).

In one embodiment, the disorder is: asthma.

In one embodiment, the disorder is: an allergy.

Raymond et al., 2009, demonstrate that the CD47-SIRPα axis is important for Th2 chronic inflammation and that this is attenuated in CD47 deficiency. Accordingly, isoQC and/or QC inhibitors, such as those described herein, may be useful therapeutic agents for the treatment of COPD, asthma, and allergies.

### Parasitic Infections

In one embodiment, the disorder is: a parasitic infection.

In one embodiment, the disorder is: malaria.

Nagaoka et al., 2019, demonstrate an interaction between a malarial protein and erythrocyte CD47 is important for malaria infection. As this interaction may depend on CD47 to be pyroglutamylated, isoQC and/or QC inhibitors, such as those described herein, may be useful therapeutic agents for the treatment of parasitic infections, for example, malaria.

### Sickle-Cell Anemia

In one embodiment, the disorder is: sickle-cell anemia.

TSP-1 interaction with CD47 of sickle cell anemia patients makes the RBCs adhere to the vascular wall, which causes vaso-occlusion and other problems. Novelli et al., June 2019, show that interfering with this may be of therapeutic use. As the interaction between TSP-1 and CD47 may be dependent on pyroglutamylation, isoQC and/or QC inhibitors, such as those described herein, may be useful therapeutic agents for the treatment of sickle-cell anemia.

### Huntington's Disease

In one embodiment, the disorder is: Huntington's disease.

Jimenez-Sanchez et al., 2015, describes how glutaminyl cyclase inhibition suppresses mutant HTT induced toxicity. Accordingly, isoQC and/or QC inhibitors, such as those described herein, may be useful therapeutic agents for the treatment of Huntington's disease.

Ischemia/Reperfusion Injury (renal/myocardial/liver/cerebral)

In one embodiment, the disorder is: ischemia or reperfusion injury (also known as ischemia-reperfusion injury).

In one embodiment, the disorder is: ischemia.

In one embodiment, the disorder is: reperfusion injury.

In one embodiment, the disorder is: renal ischemia or reperfusion injury; myocardial ischemia or reperfusion injury; liver ischemia or reperfusion injury; or cerebral ischemia or reperfusion injury.

In one embodiment, the disorder is: renal ischemia.

In one embodiment, the disorder is: renal reperfusion injury.

In one embodiment, the disorder is: myocardial ischemia.

In one embodiment, the disorder is: myocardial reperfusion injury.

In one embodiment, the disorder is: liver ischemia.

In one embodiment, the disorder is: liver reperfusion injury.

In one embodiment, the disorder is: cerebral ischemia.

In one embodiment, the disorder is: cerebral reperfusion injury.

When the supply of oxygen is returned after ischemia, ensuing tissue damage leads to prolonged inflammation if the damaged cells are not efficiently removed by phagocytosis. The efficiency of this removal can be greatly inhibited by active CD47-SIRPa signaling, and the inhibition of this axis can therefore limit reperfusion injury after ischemia. See, e.g., Li et al., April 2019; Isenberg et al., 2018; Xu et al., 2017; Wang et al., 2017; Zhang et al., 2017; Wang et al., 2016; Xiao et al., 2016; Rogers et al., 2016; Xiao et al., 2014; Lin et al., 2014; Zhou et al., 2014; Rogers et al., 2012; Jin et al., 2009; Isenberg et al., 2007. Accordingly, isoQC and/or QC inhibitors, such as those described herein, may be useful therapeutic agents for the treatment of ischemia and reperfusion injury.

### Treatment

The term “treatment,” as used herein in the context of treating a disorder, pertains generally to treatment of a human or an animal (e.g., in veterinary applications), in which some desired therapeutic effect is achieved, for example, the inhibition of the progress of the disorder, and includes a reduction in the rate of progress, a halt in the rate of progress, alleviation of symptoms of the disorder, amelioration of the disorder, and cure of the disorder. Treatment as a prophylactic measure (i.e., prophylaxis) is also included. For example, use with patients who have not yet developed the disorder, but who are at risk of developing the disorder, is encompassed by the term “treatment.”

For example, treatment of cancer includes the prophylaxis of cancer, reducing the incidence of cancer, alleviating the symptoms of cancer, etc.

The term “therapeutically-effective amount,” as used herein, pertains to that amount of a compound, or a material, composition, or dosage form comprising a compound, which is effective for producing some desired therapeutic effect, commensurate with a reasonable benefit/risk ratio, when administered in accordance with a desired treatment regimen.

### Combination Therapies

The FRPPO compounds described herein may be used as monotherapies, for example where inhibition of pyroglutamylation of CD47 or other proteins provides a therapeutic benefit per se, as described herein e.g. to promote phagocytosis.

However, the term “treatment” includes combination treatments and therapies, in which two or more treatments or therapies are combined, for example, sequentially or simultaneously. For example, the FRPPO compounds described herein may also be used in combination therapies, e.g., in conjunction with other agents.

Combination treatments or therapies include (without limitation) the following combinations of pairs of agents or modalities. It will be understood that second agents or modalities may fall into one or more of these categories.
FRPPO compounds and second agents which target (inhibit) the CD47-SIRPα signaling axis, such as agents which bind to CD47 or SIRPα. Such combinations may provide enhanced inhibition of that axis. Non-limiting examples include antibodies such as anti-CD47 antibodies and anti-SIRPα antibodies, and recombinant Fc-fusion proteins such as CD47-Fc and SIRPa-Fc;
FRPPO compounds and second agents which induce prophagocytic signals whether by virtue of the CD47-SIRPα signaling axis or otherwise, such as treatments based on Ionising Radiation (IR) or anthracyclines or anti-CD38 antibodies. Non-limiting examples include the anthracycline derivatives Doxorubicin (DOX), Daunarubicin (DNR), Epirubicin (EPI) and Idarubicin (IDA) Daratumumab;
FRPPO compounds and second agents which are independently suitable for treating any of the indications described herein. Such combinations may provide enhanced efficacy in treating such indications, particularly by targeting different aetiologies of the disorders. Non-limiting examples include Temozolomide (TMZ), used to treat, e.g., glioblastoma multiforme (GBM); Carfilzomib (Kyprolis®); Azacytidine; decitabine, etc.
FRPPO compounds and second agents which are large molecules such as proteins, e.g., antibodies, or different treatment modalities such as IR. Combinations of different types of treatments administered in different ways may provide advantages in terms of tolerance and compliance. Non-limiting examples include not only antibodies approved for use in the relevant indications, but also antibodies induced by vaccines, anti neoantigen antibodies, antibodies which have proven safe but ineffective as monotherapies; antibodies used at sub-effective (when used as monotherapy) dosages.

Combination treatments or therapies also include (without limitation) triple combinations of agents or modalities. Second and third agents may each fall into one or more of the categories of second agents described above.

In one embodiment FRPPO compounds are used to complement or enhance the effects of a monotherapy therapeutic antibody treatment, or of IR.

In one embodiment FRPPO compounds are used in combination with a therapeutic antibody which is an anti-CD47 antibody and/or anti-SIRPα antibody. Examples include Hu5F9-G4, ALX148, CC-95251, CC-90002, and IBI-188.

In one embodiment FRPPO compounds are used in combination with a recombinant Fc-fusion protein. Examples include TTI-621 and TTI-622.

In one embodiment FRPPO compounds are used in combination with a therapeutic antibody which is a PD1 or PD-L1 inhibitor such as an anti PD1 or anti PD-L1 antibody. Examples include Atezolizumab, Avelumab, and Durvalumab.

In one embodiment FRPPO compounds are used in combination with a therapeutic antibody which is selected from the list consisting of: an anti-Her2 antibody, an anti-EGFR antibody, and an anti-PDGFR antibody; an anti-GD2 (Ganglioside G2) antibody. Examples include Dinutuximab, Olaratumab, Trastuzumab, Pertuzumab, Ertumaxomab, Cetuximab, Necitumumab, Nimotuzumab, Panitumumab. Such combinations may be particularly beneficial when targeting solid tumors.

In one embodiment FRPPO compounds are used in combination with a therapeutic antibody which is selected from the list consisting of: an anti-CD19 antibody; an anti-CD20 antibody; an anti-CD38 antibody; an anti-SLAMF7 antibody; an anti-CCR40 antibody. Examples include Rituximab, Tafasitamab, Daratumumab, Elotuzumab, Mogamulizumab, Ofatumumab, Tositumomab, Obinutuzumab. Such combinations may be particularly beneficial when targeting liquid tumors.

In one embodiment FRPPO compounds are used in combination with a therapeutic antibody which is an anti-CD56 antibody or an anti-CD271-sporin antibody.

In one embodiment FRPPO compounds are used in combination with a therapeutic modality shown in the following table, i.e., as a double or triple combination, optionally for treating the disorder (“indication”) shown therein. See, e.g., see Uger et al., 2020.

Combination Therapies

| Compound | | | | Combination |
|---|---|---|---|---|
| (Company) | Target | Format | Indication | Cohorts |
| Hu5F9-G4 | CD47 | Antibody | Solid tumors | N/A |
| (Forty Seven) | | | AML, MDS | N/A |
| | | | B-cell NHL | Rituximab |
| | | | Solid tumors | Cetuximab |
| | | | and colorectal | |
| | | | cancer | |
| | | | Hematological | Azacitidine |
| | | | malignancies | |
| | | | NHL | Rituximab + |
| | | | | Acalabrutinib |
| | | | Ovarian cancer | Avelumab |
| | | | Urothelial | Atezolizumab |
| | | | carcinoma | |
| | | | AML | Atezolizumab |
| CC-90002 | CD47 | Antibody | AML, MDS | N/A |
| (Celgene) | | | Solid and | Rituximab |
| | | | hematologic | |
| | | | cancers | |
| TTI-621 | CD47 | SIRPαFc | Hematologic | Rituximab |
| (Trillium | | | malignancies, | Nivolumab |
| Therapeutics) | | | selected solid | |
| | | | tumors | |
| | | | Solid tumors, | Anti-PD-1/PD- |
| | | | mycosis | L1 Peg IFN-α2a |
| | | | fungoides | T-Vec |
| | | | | Radiation |
| ALX148 (ALX | CD47 | Mutated | Solid tumors, | Pembrolizumab |
| Oncology) | | SIRPαFc | lymphoma | Trastuzumab |
| | | | | Rituximab |
| IBI188 | CD47 | Antibody | Advanced | Rituximab |
| (Innovent | | | malignancies | |
| Biologics) | | | Advanced | N/A |
| | | | malignant | |
| | | | tumors and | |
| | | | lymphoma | |
| SRF231 | CD47 | Antibody | Solid and | N/A |
| (Surface | | | hematologic | |
| Oncology) | | | cancers | |
| TTI-622 | CD47 | SIRPαFc | Lymphoma, | Rituximab Anti- |
| (Trillium | | | multiple | PD-1/PD-L1 |
| Therapeutics) | | | myeloma | Proteasome- |
| | | | | Inhibitor |
| CC-95251 | SIRPα | Antibody | Solid tumors | Cetuximab |
| (Celgene) | | | | |
| AO-176 (Arch | CD47 | Antibody | Solid tumors | N/A |
| Oncology) | | | | |
| TJ011133 (I- | CD47 | Antibody | Solid tumors, | Pembrolizumab |
| Mab | | | lymphoma | Rituximab |
| Biopharma) | | | | |
| TG-1801 (TG | CD47/CD19 | Bispecific | B-cell | N/A |
| Therapeutics) | | antibody | lymphoma | |
| BI 765063 | SIRPα | Antibody | Solid tumors | Anti-PD-1 |
| (Boehringer | | | | |
| Ingelheim/OSE) | | | | |
| SGN-CD47M | CD47 | Antibody | Solid tumors | N/A |
| (Seattle | | | | |
| Genetics) | | | | |

As used herein, the term “antibody” is used in a general sense to include any polypeptide or protein comprising an antibody antigen-binding site described herein, including Fab, Fab₂, Fab₃, diabodies, triabodies, tetrabodies, minibodies and single-domain antibodies, as well as whole antibodies of any isotype or sub-class.

Both monospecific and bispecific antibodies are included. An example of a bispecific antibody is an anti-CD20-CD47 bispecific antibody or anti-CD19-CD47 bispecific antibody.

An antibody may, for example, be a single-chain variable fragment (scFv) or single-chain antibody (scAb). An scFv fragment is a fusion of a variable heavy (VH) and variable light (VL) chain. A scAb has a constant light (CL) chain fused to the VL chain of an scFv fragment. The CL chain is optionally the human kappa light chain (HuCκ). A single chain Fv (scFv) may be comprised within a mini-immunoglobulin or small immunoprotein (SIP), e.g., as described in Li et al., 1997. A SIP may comprise an scFv molecule fused to the CH4 domain of the human IgE secretory isoform IgE-S2 (ε_{S2}-CH4; see, e.g., Batista et al., 1996) forming an homo-dimeric mini-immunoglobulin antibody molecule.

Antibodies and methods for their construction and use are known in the art and described, in for example, Holliger et al., 2005 and Liu et al., 2020.

Antibodies used in the treatments herein may lack antibody constant regions.

However in preferred embodiments, antibodies are whole antibodies. For example, the antibody may be an IgG, IgA, IgE or IgM or any of the isotype sub-classes, particularly IgG1.

In one embodiment FRPPO compounds are used in combination with a therapeutic antibody which is a monoclonal antibody, optionally a human or humanised monoclonal antibody.

In one embodiment FRPPO compounds are used in combination with a therapeutic antibody which is an IgG antibody.

In one embodiment FRPPO compounds are used in combination with a therapeutic antibody which is an IgA antibody.

One aspect of the present invention pertains to a FRPPO compound as described herein, in combination with one or more (e.g., 1, 2, 3, 4, etc.) additional therapeutic agents.

The particular combination would be at the discretion of the physician who would select dosages using their common general knowledge and dosing regimens known to a skilled practitioner.

The agents (e.g., the FRPPO compound as described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

The agents (e.g., the FRPPO compound described here, plus one or more other agents) may be formulated together in a single dosage form, or alternatively, the individual agents may be formulated separately, and may optionally be presented together in the form of a kit, optionally with instructions for their use.

### Other Uses

The FRPPO compounds described herein may also be used as cell culture additives to inhibit glutaminyl-peptide cyclotransferase-like (isoQC) enzyme and/or glutaminyl-peptide cyclotransferase (QC) enzyme (e.g., to inhibit or reduce or block the activity or function of isoQC and/or QC enzyme).

The FRPPO compounds described herein may also be used as part of an in vitro assay, for example, in order to determine whether a candidate host is likely to benefit from treatment with the compound in question.

The FRPPO compounds described herein may also be used as a standard, for example, in an assay, in order to identify other active compounds, other isoQC and/or QC enzyme inhibitors, etc.

### Kits

Another aspect of the present invention pertains to a kit comprising (a) a FRPPO compound, as described herein, preferably provided as a composition (e.g., a pharmaceutical composition) and in a suitable container and/or with suitable packaging; and (b) instructions for use, for example, in a method of treatment of a disorder (e.g., a disease) as described herein, for example, written instructions on how to administer the compound.

The written instructions may also include a list of indications for which the FRPPO compound is a suitable treatment.

### Routes of Administration

The FRPPO compound or pharmaceutical composition comprising the FRPPO compound may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### The Subject/Patient

Groups, sub-groups or cohorts of subjects/patients who may particularly benefit from the treatments of the present invention may be selected for treatment with one or more FRPPO compounds. Such selection may be performed as an active step in any of the aspects or embodiments of the invention relating to treatment. For example such subjects/patients may be those suffering a disorder which may be associated with aberrant expression (e.g., upregulation) of CD47 on diseased or other undesirable cells, and the existence of such aberrant expression may be used as a selection criterion. Selected subjects/patients may additionally or alternatively be those who would benefit from reduced signalling or binding between CD47 on the surface of a first cell and SIRPα on the surface of a second cell e.g. where it is undesirable to utilise antibody therapies, or other protein or large biomolecule therapies, targeting CD47 and/or SIRPα e.g. as the subject/patient is refractory to such therapies, or they are otherwise unsuitable for the subject/patient (for example for the reasons explained in the section entitled “The CD47-SIRPα Signalling Axis” above). Since the FRPPO compounds may demonstrate benefit via mechanisms such as phagocytosis, ADCC or ADCP, subjects/patients may additionally or alternatively be selected according to immune status to ensure such immunotherapies are most likely to succeed—for example subjects/patients having or demonstrating high levels of prophagocytic signals/macrophage infiltration e.g. in disease tissues.

The subject/patient may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutan, gibbon), or a human.

Furthermore, the subject/patient may be any of its forms of development, for example, a foetus.

In one preferred embodiment, the subject/patient is a human.

### Formulations

While it is possible for a FRPPO compound to be administered alone, it is preferable to present it as a pharmaceutical formulation (e.g., composition, preparation, medicament) comprising at least one FRPPO compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, also described herein are pharmaceutical compositions, as defined above, and methods of making a pharmaceutical composition comprising mixing at least one FRPPO compound, as described herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington: The Science and Practice of Pharmacy, 21st edition, Lippinott Williams and Wilkins, 2005; Remington: The Science and Practice of Pharmacy, 22nd edition, Pharmaceutical Press, 2012; and Handbook of Pharmaceutical Excipients, 7th edition, Pharmaceutical Press, 2012.

The formulations may be prepared by any methods well known in the art of pharmacy. Such methods include the step of bringing into association the compound with a carrier which constitutes one or more accessory ingredients. In general, the formulations are prepared by uniformly and intimately bringing into association the compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, losenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Formulations may suitably be provided as a patch, adhesive plaster, bandage, dressing, or the like which is impregnated with one or more compounds and optionally one or more other pharmaceutically acceptable ingredients, including, for example, penetration, permeation, and absorption enhancers. Formulations may also suitably be provided in the form of a depot or reservoir.

The compound may be dissolved in, suspended in, or mixed with one or more other pharmaceutically acceptable ingredients. The compound may be presented in a liposome or other microparticulate which is designed to target the compound, for example, to blood components or one or more organs.

Formulations suitable for oral administration (e.g., by ingestion) include liquids, solutions (e.g., aqueous, non-aqueous), suspensions (e.g., aqueous, non-aqueous), emulsions (e.g., oil-in-water, water-in-oil), elixirs, syrups, electuaries, tablets, granules, powders, capsules, cachets, pills, ampoules, boluses.

Formulations suitable for buccal administration include mouthwashes, losenges, pastilles, as well as patches, adhesive plasters, depots, and reservoirs. Losenges typically comprise the compound in a flavoured basis, usually sucrose and acacia or tragacanth. Pastilles typically comprise the compound in an inert matrix, such as gelatin and glycerin, or sucrose and acacia. Mouthwashes typically comprise the compound in a suitable liquid carrier.

Formulations suitable for sublingual administration include tablets, losenges, pastilles, capsules, and pills.

Formulations suitable for oral transmucosal administration include liquids, solutions (e.g., aqueous, non-aqueous), suspensions (e.g., aqueous, non-aqueous), emulsions (e.g., oil-in-water, water-in-oil), mouthwashes, losenges, pastilles, as well as patches, adhesive plasters, depots, and reservoirs.

Formulations suitable for non-oral transmucosal administration include liquids, solutions (e.g., aqueous, non-aqueous), suspensions (e.g., aqueous, non-aqueous), emulsions (e.g., oil-in-water, water-in-oil), suppositories, pessaries, gels, pastes, ointments, creams, lotions, oils, as well as patches, adhesive plasters, depots, and reservoirs.

Formulations suitable for transdermal administration include gels, pastes, ointments, creams, lotions, and oils, as well as patches, adhesive plasters, bandages, dressings, depots, and reservoirs.

Tablets may be made by conventional means, e.g., compression or moulding, optionally with one or more accessory ingredients. Compressed tablets may be prepared by compressing in a suitable machine the compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); preservatives (e.g., methyl p-hydroxybenzoate, propyl p-hydroxybenzoate, sorbic acid); flavours, flavour enhancing agents, and sweeteners. Moulded tablets may be made by moulding in a suitable machine a mixture of the powdered compound moistened with an inert liquid diluent. The tablets may optionally be coated or scored and may be formulated so as to provide slow or controlled release of the compound therein using, for example, hydroxypropylmethyl cellulose in varying proportions to provide the desired release profile. Tablets may optionally be provided with a coating, for example, to affect release, for example an enteric coating, to provide release in parts of the gut other than the stomach.

Ointments are typically prepared from the compound and a paraffinic or a water-miscible ointment base.

Creams are typically prepared from the compound and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the compound through the skin or other affected areas. Examples of such dermal penetration enhancers include dimethylsulfoxide and related analogues.

Emulsions are typically prepared from the compound and an oily phase, which may optionally comprise merely an emulsifier (otherwise known as an emulgent), or it may comprise a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulfate. The choice of suitable oils or fats for the formulation is based on achieving the desired cosmetic properties, since the solubility of the compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for intranasal administration, where the carrier is a liquid, include, for example, nasal spray, nasal drops, or by aerosol administration by nebuliser, include aqueous or oily solutions of the compound.

Formulations suitable for intranasal administration, where the carrier is a solid, include, for example, those presented as a coarse powder having a particle size, for example, in the range of about 20 to about 500 microns which is administered in the manner in which snuff is taken, i.e., by rapid inhalation through the nasal passage from a container of the powder held close up to the nose.

Formulations suitable for pulmonary administration (e.g., by inhalation or insufflation therapy) include those presented as an aerosol spray from a pressurised pack, with the use of a suitable propellant, such as dichlorodifluoromethane, trichlorofluoromethane, dichorotetrafluoroethane, carbon dioxide, or other suitable gases.

Formulations suitable for ocular administration include eye drops wherein the compound is dissolved or suspended in a suitable carrier, especially an aqueous solvent for the compound.

Formulations suitable for rectal administration may be presented as a suppository with a suitable base comprising, for example, natural or hardened oils, waxes, fats, semi-liquid or liquid polyols, for example, cocoa butter or a salicylate; or as a solution or suspension for treatment by enema.

Formulations suitable for vaginal administration may be presented as pessaries, tampons, creams, gels, pastes, foams or spray formulations containing in addition to the compound, such carriers as are known in the art to be appropriate.

Formulations suitable for parenteral administration (e.g., by injection), include aqueous or non-aqueous, isotonic, pyrogen-free, sterile liquids (e.g., solutions, suspensions), in which the compound is dissolved, suspended, or otherwise provided (e.g., in a liposome or other microparticulate). Such liquids may additionally contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the compound in the liquid is from about 1 ng/mL to about 10 μg/mL, for example from about 10 ng/mL to about 1 μg/mL. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

### Dosage

It will be appreciated by one of skill in the art that appropriate dosages of the FRPPO compounds, and compositions comprising the FRPPO compounds, can vary from patient to patient. Determining the optimal dosage will generally involve the balancing of the level of therapeutic benefit against any risk or deleterious side effects. The selected dosage level will depend on a variety of factors including, but not limited to, the activity of the particular FRPPO compound, the route of administration, the time of administration, the rate of excretion of the FRPPO compound, the duration of the treatment, other drugs, compounds, and/or materials used in combination, the severity of the disorder, and the species, sex, age, weight, condition, general health, and prior medical history of the patient. The amount of FRPPO compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the FRPPO compound is in the range of about 0.01 mg to about 5000 mg (more typically about 0.1 mg to about 300 mg) per kilogram body weight of the subject per day.

Where the compound is a salt, an ester, an amide, a prodrug, or the like, the amount administered is calculated on the basis of the parent compound and so the actual weight to be used is increased proportionately.

### EXAMPLES

### Chemical Synthesis

### Abbreviations

ACN—acetonitrile
BAST—bis(2-methoxyethyl)aminosulfur trifluoride
Boc—tert-butoxycarbonyl
dba—dibenzylidineacetone
DCM—dichloromethane
DEA—diethylamine
DIBAL, DIBAL-H—diisobutylaluminum hydride

### DIEA—N,N-diisopropylethylamine

DMEDA—N,N′-dimethylethylene-1,2-diamine

### DMF—N,N-dimethylformamide

DMSO—dimethylsulfoxice
dppf—1,1′-bis(diphenylphosphino)ferrocene
EDA—Ethylene-1,2-diamine
FCC—flash column chromatography
HPLC—high performance liquid chromatography
IBX—2-iodoxybenzoic acid
LDA—lithium diisopropylamide
LiHMDS—lithium hexamethyldisilazane

### NBS—N-bromosuccinimide

NMR—nuclear magnetic resonance spectroscopy
PCC—pyridinium chlorochromate
PG—protecting group
RuPhos—2-dicyclohexylphosphino-2′,6′-diisopropoxybiphenyl
SEMCl—2-(trimethylsilyl)ethoxymethyl chloride
SFC—supercritical fluid chromatography
TFA—trifluoroacetic acid
THF—tetrahydrofuran
XantPhos—4,5-bis(diphenylphosphino)-9,9-dimethylxanthene
XPhos—2-dicyclohexylphosphino-2′,4′,6′-triisopropylbiphenyl

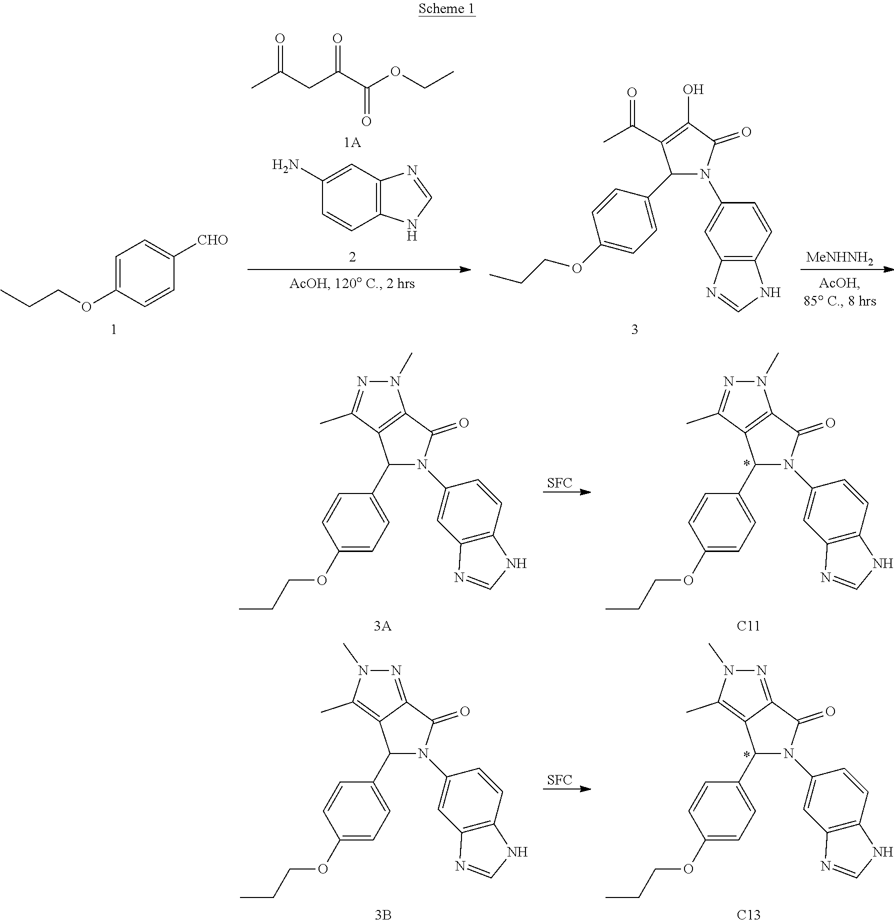

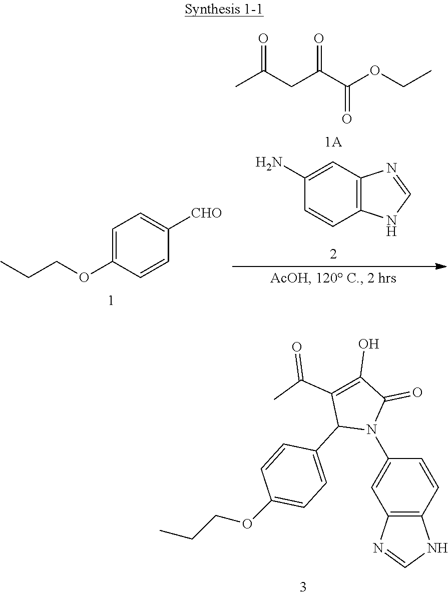

To a solution of compound 1 (0.500 g, 3.05 mmol, 481 μL, 1.00 eq) in AcOH (5 mL) was added compound 2 (405 mg, 3.05 mmol, 1.00 eq) and ethyl compound 1A (481 mg, 3.05 mmol, 429 μL, 1.00 eq). The mixture was stirred at 120° C. for 2 hours. LCMS showed that the desired mass was detected. The crude product was purified by reversed-phase HPLC (0.1% formic acid H₂O/MeCN). Compound 3 (320 mg, 817 μmol, 26.9% yield) was obtained as a solid and used without further purification

LCMS: RT=0.955 min. m/z=392 (M+1)⁺.

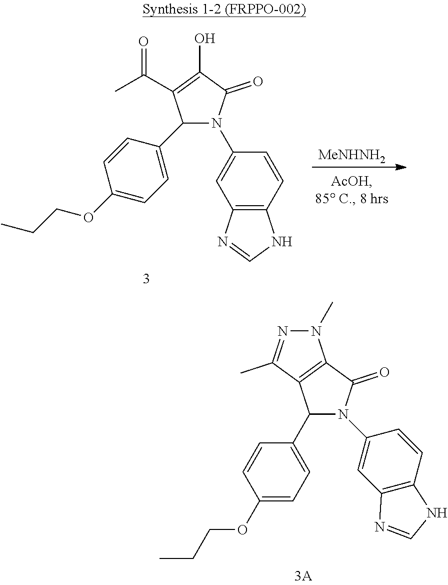 [SMILES: "MeNHNH2" ; CCCOc1ccc(C2C(C(C)=O)=C(O)C(=O)N2c2ccc3[nH]cnc3c2)cc1 ; CCCOc1ccc(C2c3c(C)nn(C)c3C(=O)N2c2ccc3[nH]cnc3c2)cc1]

To a solution of compound 3 (0.320 g, 817 μmol, 1.00 eq) in AcOH (5 mL) was added methylhydrazine (0.180 g, 1.56 mmol, 205 μL, 1.91 eq). The mixture was stirred at 85° C. for 8 hours. TLC (ethyl acetate) showed two major spots. The reaction mixture was concentrated and then diluted with EtOAc (20 mL). The resulting solution was washed with saturated NaHCO₃ solution (20 mL×2) and saturated brine (20 mL×2), dried over anhydrous Na₂SO₄, filtered and concentrated to give a residue which was purified by prep-TLC (SiO₂, ethyl acetate) and the spot with Rf=0.45 was isolated. Compound 3A (0.150 g, 366 μmol, 44.8% yield, 98.0% purity) was obtained as a yellow solid. The identity of the pyrazole regioisomer was confirmed by 1D- and 2D-¹H NMR.

¹H NMR: 400 MHz CDCl₃: δ 7.77 (s, 1H), 7.51 (s, 1H), 7.43-7.41 (m, 1H), 7.08-7.01 (m, 3H), 6.74-6.64 (m, 2H), 5.81 (s, 1H), 4.09 (s, 3H), 3.85-3.79 (m, 2H), 2.07 (s, 3H), 1.78-1.69 (m, 2H), 0.98 (t, J=7.4 Hz, 3H).

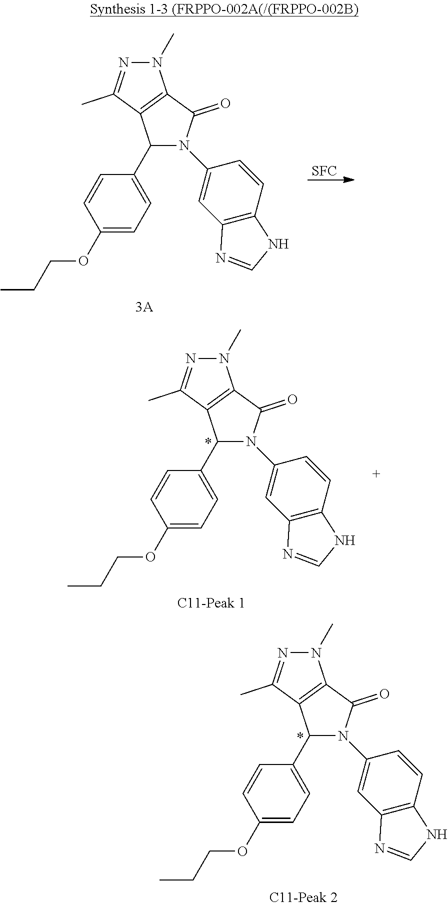

Compound 3A (150 mg, 366 μmol, 1.00 eq) was purified by multiple injections on chiral SFC (column DAICEL CHIRALPAK AD 250×30 mm, 10 μm; mobile phase A—supercritical CO₂, B-iPrOH (0.1% NH₄OH); isocratic 30% B, 10 min).

C11-Peak 1 (RT=4.7 min, 39.5 mg, 98.5 μmol, 53.8% yield, 100% purity) was obtained as a light yellow solid, which was confirmed by ¹H NMR, LCMS, and SFC.

LCMS: RT=2.38 min, m/z=402 (M+1)⁺.

Chiral SFC: RT=1.724 min.

¹H NMR: 400 MHz CDCl₃: δ 7.85 (s, 1H), 7.54 (s, 1H), 7.47 (s, 1H), 7.14-7.12 (m, 1H), 7.04-7.01 (m, 2H), 6.74-6.72 (m, 2H), 5.83 (s, 1H), 4.09 (s, 3H), 3.86-3.78 (m, 2H), 2.07 (s, 3H), 1.79-1.72 (m, 2H), 1.06-0.97 (m, 3H).

C11-Peak 2 (RT=8.4 min, 39.8 mg, 99.2 μmol, 54.2% yield, 100% purity) was obtained as a light yellow solid, which was confirmed by ¹H NMR, LCMS, and SFC.

LCMS: RT=2.34 min, m/z=402 (M+1)⁺.

Chiral SFC: RT=1.946 min.

¹H NMR: 400 MHz CDCl₃: δ 7.81 (s, 1H), 7.52 (s, 1H), 7.45 (s, 1H), 7.11-7.09 (m, 1H), 7.04-7.02 (m, 2H), 6.74-6.72 (m, 2H), 5.82 (s, 1H), 4.09 (s, 3H), 3.87-3.77 (m, 2H), 2.07 (s, 3H), 1.77-1.72 (m, 2H), 1.06-0.97 (m, 3H).

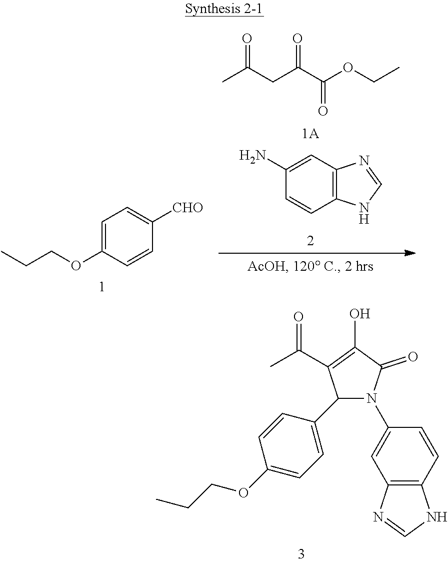 [SMILES: CCOC(=O)C(=O)CC(C)=O ; CCCOc1ccc(C=O)cc1 ; Nc1ccc2[nH]cnc2c1 ; CCCOc1ccc(C2C(C(C)=O)=C(O)C(=O)N2c2ccc3[nH]cnc3c2)cc1]

A mixture of compound 1A (481 mg, 3.05 mmol, 429 μL, 1.00 eq), compound 1 (0.500 g, 3.05 mmol, 480 μL, 1.00 eq) and compound 2 (405 mg, 3.05 mmol, 1.00 eq) in AcOH (5 mL) was degassed and purged with N₂ 3 times, and then the mixture was stirred at 120° C. for 2 hours under N₂ atmosphere. LCMS showed that the desired mass was present. The crude product was purified by reversed-phase HPLC (0.1% formic acid H₂O/MeCN). Compound 3 (250 mg, 638 μmol, 20.9% yield) was obtained as a solid and used without further purification.

LCMS: RT=0.787 min. m/z=392 (M+1)⁺.

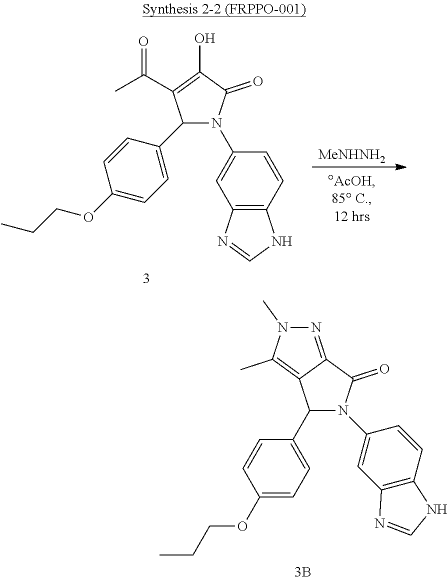

To a solution of compound 3 (0.200 g, 511 μmol, 1.00 eq) in AcOH (5 mL) was added methylhydrazine (0.210 g, 4.56 mmol, 240 μL, 8.92 eq). The mixture was stirred at 85° C. for 12 hours. TLC (ethyl acetate) showed two major spots. The reaction mixture was concentrated and then diluted with EtOAc (20 mL). The resulting solution was washed with saturated NaHCO₃ solution (20 mL×2) and saturated brine (20 mL×2), dried over anhydrous Na₂SO₄, filtered and concentrated to residue which was purified by prep-TLC (SiO₂, ethyl acetate) and the spot with Rf=0.20 was isolated. Compound 3B (90 mg, 221 μmol, 43.4% yield, 98.9% purity) was obtained as a yellow solid. The identity of the pyrazole regioisomer was confirmed by 1D- and 2D-¹H NMR.

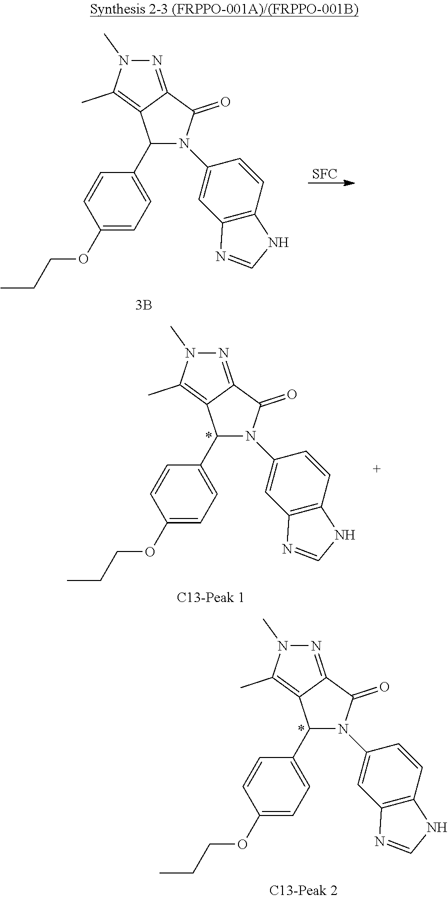

Compound 3B (90 mg, 224 μmol, 1.00 eq) was concentrated under reduced pressure to give a residue which was purified by multiple injections on chiral SFC (column DAICEL CHIRALPAK AD 250×30 mm, 10 μm; mobile phase A—supercritical CO₂, B—MeOH (0.1% NH₄OH); isocratic 40% B, 7 min).

C13-Peak 1 (RT=3.0 min, 34.3 mg, 85.4 μmol, 76.13% yield, 100% purity) was obtained as a light yellow solid, which was confirmed by ¹H NMR, LCMS, and SFC.

LCMS: RT=1.79 min, m/z=402 (M+1)⁺.

Chiral SFC: RT=1.445 min.

¹H NMR: 400 MHz MeOD: δ 8.14 (s, 1H), 7.56 (s, 2H), 7.28-7.26 (m, 1H), 7.08-7.06 (m, 2H), 6.77-6.75 (m, 2H), 6.14 (s, 1H), 3.93 (s, 3H), 3.83-3.80 (m, 2H), 2.14 (s, 3H), 1.75-1.67 (m, 2H), 0.99-0.95 (m, 3H).

C13-Peak 2 (RT=4.1 min, 27.2 mg, 67.7 μmol, 60.4% yield, 100% purity) was obtained as a yellow solid, which was confirmed by ¹H NMR, LCMS, and SFC.

LCMS: RT=1.77 min, m/z=402 (M+1)⁺.

Chiral SFC: RT=1.693 min.

¹H NMR: 400 MHz MeOD: δ 8.13 (s, 1H), 7.57-7.52 (m, 2H), 7.28-7.25 (m, 1H), 7.08-7.06 (m, 2H), 6.77-6.75 (m, 2H), 6.13 (s, 1H), 3.92 (s, 3H), 3.83-3.77 (m, 2H), 2.14 (s, 3H), 1.75-1.66 (m, 2H), 0.99-0.95 (m, 3H).

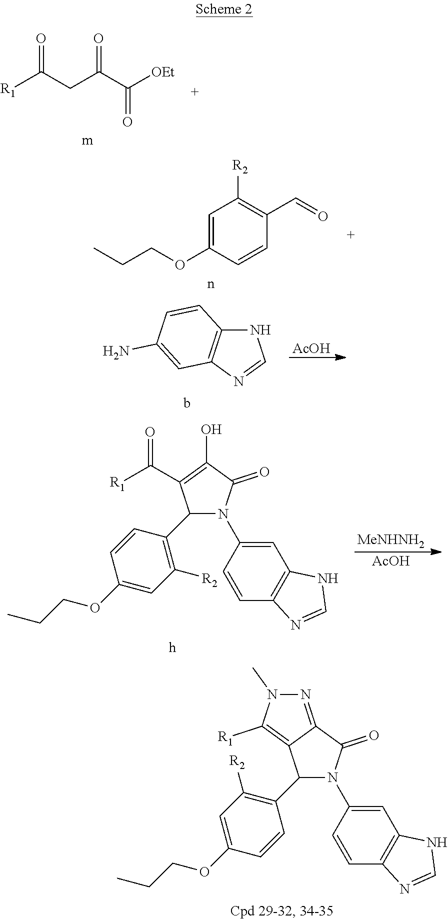

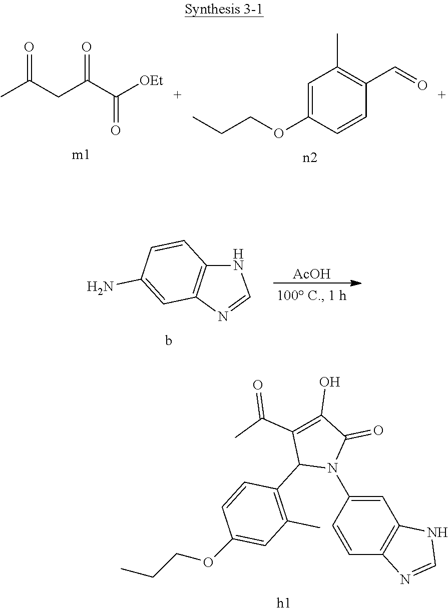 [SMILES: "AcOH" ; CCOC(=O)C(=O)CC(C)=O ; CCCOc1ccc(C=O)c(C)c1 ; Nc1ccc2[nH]cnc2c1 ; CCCOc1ccc(C2C(C(C)=O)=C(O)C(=O)N2c2ccc3nc[nH]c3c2)c(C)c1]

The mixture of compound n2 (669 mg, 3.76 mmol, 1.00 eq), compound m1 (594 mg, 3.76 mmol, 530 μL, 1.00 eq) and compound b (500 mg, 3.76 mmol, 1.00 eq) in AcOH (5 mL) was stirred at 100° C. for 1 hour. LCMS detected the desired product mass. The mixture was concentrated under reduced pressure to give a residue. The crude product was purified by reversed-phase HPLC (0.1% formic acid H₂O/MeCN) to give compound h1 (200 mg, 493 μmol, 13.1% yield) as a black brown solid which was used without further purification.

LCMS: RT=0.802 min, m/z=406.1 (M+1)⁺.

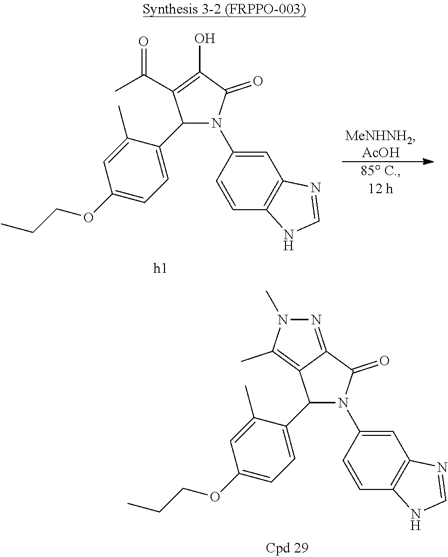

To a solution of compound h1 (250 mg, 617 μmol, 1.00 eq) in AcOH (10 mL) was added methylhydrazine (710 mg, 6.17 mmol, 812 μL, 10.0 eq) and the mixture was stirred at 85° C. for 12 hours. The mixture was concentrated under reduced pressure to give a residue which was purified by preparative HPLC (column Waters Xbridge 150×50 mm, 10 μm; mobile phase (0.05% NH₄OH) A—water, B—ACN; gradient 25-55% B, 11.5 min) to give Cpd 29 (9.45 mg, 22.70 μmol, 3.68% yield, 99.8% purity) as a yellow solid.

LCMS: RT=0.925 min, m/z=416.4 (M+1)⁺.

HPLC: RT=2.113 min.

¹H NMR: 400 MHz MeOD: δ 8.14 (s, 1H), 7.54-7.49 (m, 2H), 7.23-7.22 (m, 1H), 6.79-6.77 (m, 1H), 6.64-6.62 (m, 2H), 6.41 (s, 1H), 3.93 (s, 3H), 3.83-3.79 (m, 2H), 2.39-2.30 (m, 3H), 2.12 (s, 3H), 1.73-1.68 (m, 2H), 0.99-0.96 (m, 3H).

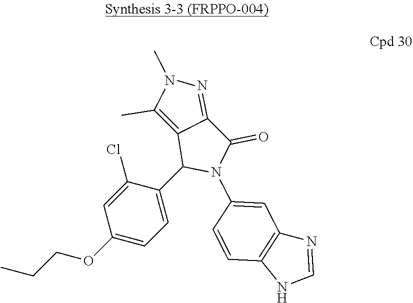 [SMILES: CCCOc1ccc(C2c3c(nn(C)c3C)C(=O)N2c2ccc3[nH]cnc3c2)c(Cl)c1]

The compound was obtained using an analogous method. Cpd 30 (20.1 mg, 45.6 μmol, 8.4% yield, 99% purity) was obtained as a yellow solid.

LCMS: RT=0.951 min, m/z=436.3 (M+1)⁺.

HPLC: RT=2.214 min.

¹H NMR: 400 MHz MeOD: δ 8.14 (s, 1H), 7.67 (s, 1H), 7.55-7.53 (m, 1H), 7.35-7.33 (m, 1H), 6.93-6.90 (m, 2H), 6.74-6.73 (m, 2H), 3.92 (s, 3H), 3.84-3.81 (m, 3H), 2.20 (s, 3H), 1.73-1.68 (m, 2H), 0.98-0.95 (m, 3H).

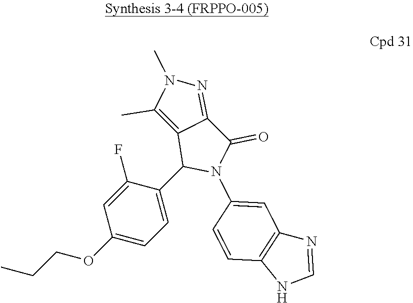 [SMILES: CCCOc1ccc(C2c3c(nn(C)c3C)C(=O)N2c2ccc3[nH]cnc3c2)c(F)c1]

The compound was obtained using an analogous method. Cpd 31 (17.09 mg, 38.7 μmol, 26% yield, 95% purity) was obtained as a yellow solid.

LCMS: RT=0.812 min, m/z=420.3 (M+1)⁺.

HPLC: RT=1.698 min.

¹H NMR: 400 MHz MeOD: δ 8.16 (s, 1H), 7.63-7.62 (m, 1H), 7.56-7.54 (d, J=8.00, 1H), 7.32-7.29 (m, 1H), 7.04-7.00 (m, 1H), 6.62-6.55 (m, 2H), 6.43 (m, 1H), 3.93 (s, 3H), 3.84-3.80 (m, 2H), 2.18-2.16 (m, 3H), 1.75-1.66 (m, 2H), 0.99-0.95 (m, 3H).

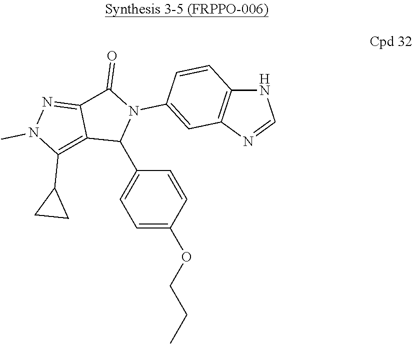 [SMILES: CCCOc1ccc(C2c3c(nn(C)c3C3CC3)C(=O)N2c2ccc3[nH]cnc3c2)cc1]

The compound was obtained using an analogous method. Cpd 32 (37.8 mg, 85.3 μmol, 7.1% yield, 96.4% purity) was obtained as a light yellow solid.

LCMS: RT=0.918 min, m/z=428.3 (M+1)⁺.

HPLC: RT=2.207 min.

¹H NMR: 400 MHz MeOH: δ 12.4 (s, 1H), 8.15 (s, 1H), 7.63-7.38 (m, 3H), 7.27-7.06 (m, 2H), 6.75-6.73 (m, 2H), 6.31 (s, 1H), 3.97 (s, 3H), 3.83-3.77 (m, 2H), 1.85-1.83 (m, 1H), 1.66-1.60 (m, 2H), 0.92-0.90 (m, 3H), 0.88-0.83 (m, 1H), 0.68-0.67 (m, 1H), 0.46-0.45 (m, 1H), 0.24-0.22 (m, 1H).

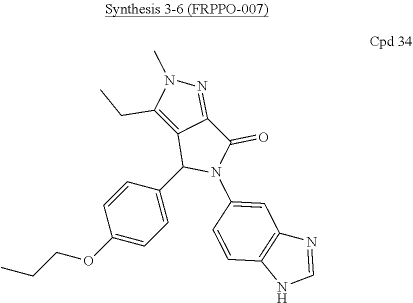 [SMILES: CCCOc1ccc(C2c3c(nn(C)c3CC)C(=O)N2c2ccc3[nH]cnc3c2)cc1]

The compound was obtained using an analogous method. Cpd 34 (11.98 mg, 28.80 μmol, 2.2% yield, 99.9% purity) was obtained as a yellow solid.

LCMS: RT=0.914 min, m/z=416.3 (M+1)⁺.

HPLC: RT=2.173 min.

¹H NMR: 400 MHz MeOD: δ 8.13 (s, 1H), 7.54 (s, 2H), 7.26-7.23 (m, 1H), 7.07-7.04 (m, 2H), 6.77-6.74 (m, 2H), 6.16 (s, 1H), 3.94 (s, 3H), 3.82-3.79 (m, 2H), 2.70-2.50 (m, 2H), 1.75-1.66 (m, 2H), 0.99-0.93 (m, 6H).

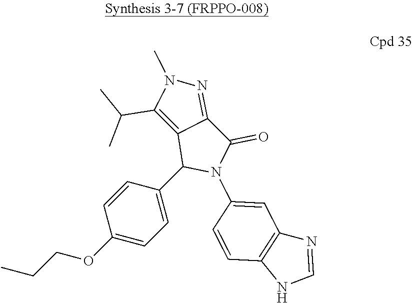 [SMILES: CCCOc1ccc(C2c3c(nn(C)c3C(C)C)C(=O)N2c2ccc3[nH]cnc3c2)cc1]

The compound was obtained using an analogous method. Cpd 35 (8.06 mg, 18.4 μmol, 3.9% yield, 97.9% purity) was obtained as a light yellow solid.

LCMS: RT=0.958 min, m/z=430.3 (M+1)⁺.

HPLC: RT=2.273 min.

¹H NMR: 400 MHz MeOD: δ 12.39 (s, 1H), 8.16-8.15 (m, 1H), 7.65-7.51 (m, 1H), 7.40-7.38 (m, 1H), 7.30-7.26 (m, 1H), 7.08-7.06 (m, 2H), 6.75-6.73 (d, J=8.00, 2H), 6.35-6.33 (d, J=8.00, 1H), 3.91 (s, 3H), 3.79-3.76 (m, 2H), 3.10-3.03 (m, 1H), 1.66-1.58 (m, 2H), 1.18-1.16 (m, 3H), 0.91-0.87 (m, 3H), 0.62-0.60 (d, J=8.00, 3H).

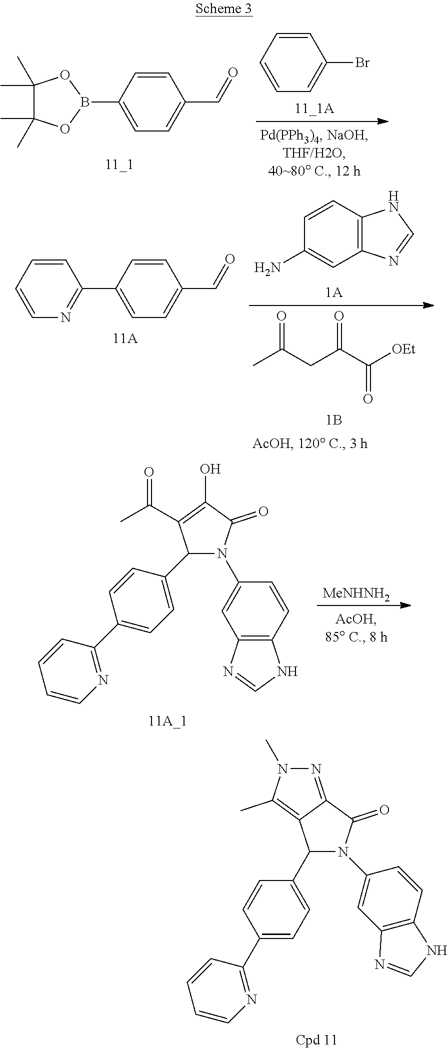

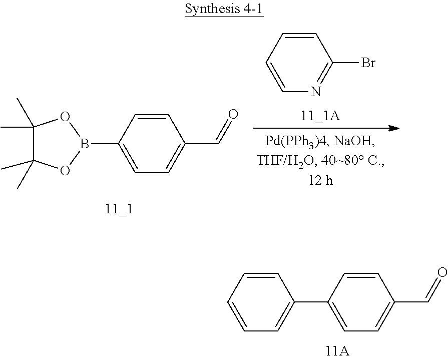 [SMILES: Brc1ccccn1 ; CC1(C)OB(c2ccc(C=O)cc2)OC1(C)C ; O=Cc1ccc(-c2ccccc2)cc1]

A mixture of compound 11_1 (3.00 g, 12.9 mmol, 1.50 eq), compound 11_1A (1.36 g, 8.62 mmol, 820 μL, 1.00 eq), Pd(PPh₃)₄ (498 mg, 431 μmol, 0.05 eq), Na₂CO₃ (2M, 12.9 mL, 3.00 eq) in dioxane (13 mL) was degassed and purged with N₂ 3 times, and then the mixture was stirred at 100° C. for 18 hours under N₂ atmosphere. LCMS showed starting material was consumed completely, and one main peak with the desired mass was detected. The reaction mixture was partitioned between water (20 mL) and ethyl acetate (20 mL). The organic phase was separated, washed with ethyl acetate (10 mL×3), dried over Na₂SO₄, filtered and concentrated under reduced pressure to give a yellow oil. The crude product was purified by column chromatography (SiO₂, petroleum ether/ethyl acetate=9/1 to 3/1, product R_{f}=0.60) to give compound 11A (1.10 g, 5.88 mmol, 68% yield, 98.0% purity) as a white solid.

LCMS: product RT=0.68 min, m/z=184.0 (M+1)⁺.

HPLC: RT=1.79 min, 98.8% purity.

¹H NMR: CDCl₃, 400 MHz: δ 10.1 (s, 1H), 8.76 (d, J=3.60 Hz, 1H), 8.19 (d, J=8.40 Hz, 2H), 8.01 (d, J=8.40 Hz, 2H), 7.84 (d, J=3.20 Hz, 2H), 7.34-7.30 (m, 1H).

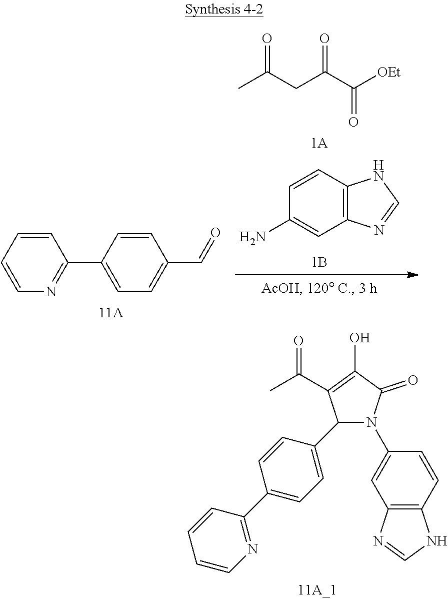

A mixture of 11A (500 mg, 2.70 mmol, 1.00 eq), 1B (457 mg, 2.70 mmol, 1.00 eq, HCl) and 1A (426 mg, 2.70 mmol, 381 μL, 1.00 eq) in AcOH (5 mL) was stirred at 120° C. for 2 hours. LCMS showed 11A was consumed completely and one main peak with the desired mass was detected. The reaction was concentrated under vacuum and the residue was purification by HPLC (mobile phase (0.1% formic acid) A—water, B—MeOH; gradient 20-40% B) to give 11A_1 (750 mg, 1.44 mmol, 53% yield, 78.8% purity) as a yellow solid. The crude product was used the next step without further purification.

LCMS: product RT=0.24 min, m/z=411.0 (M+1)⁺.

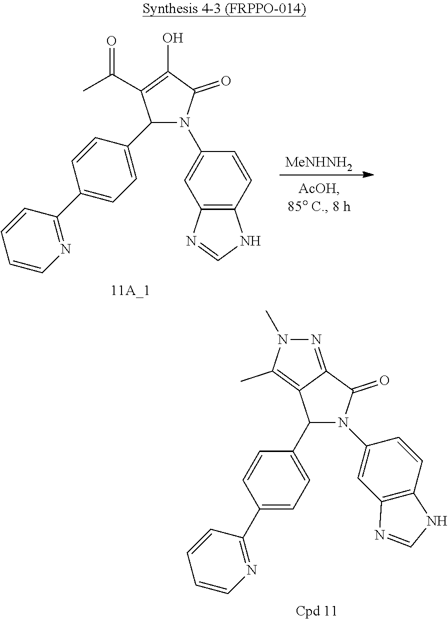 [SMILES: "MeNHNH2" ; CC(=O)C1=C(O)C(=O)N(c2ccc3[nH]cnc3c2)C1c1ccc(-c2ccccn2)cc1 ; Cc1c2c(nn1C)C(=O)N(c1ccc3[nH]cnc3c1)C2c1ccc(-c2ccccn2)cc1]

To a mixture of 11A_1 (430 mg, 826 μmol, 1.00 eq) in AcOH (5 mL) was added methylhydrazine (200 mg, 1.74 mmol, 229 μL, 2.10 eq). The mixture was stirred at 85° C. for 8 hours. LCMS showed 11A_1 was consumed completely and two main peaks with the desired mass was detected. The reaction mixture was concentrated under vacuum. The residual was purification by preparative HPLC (column Xtimate C18 150×25 mm, 5 μm; mobile phase (0.05% NH₄OH) A—water, B—ACN; gradient 24-54% B, 10 min) to give Cpd 11 (81.5 mg, 194 μmol, 24% yield, 100% purity) as a yellow solid.

LCMS: product RT=0.85 min, m/z=421.3 (M+1)⁺.

HPLC: RT=1.81 min, 95.9% purity.

¹H NMR: CDCl₃, 400 MHz: δ 8.64 (d, J=4.40 Hz, 1H), 7.91 (s, 1H) 7.86 (d, J=8.00 Hz, 2H), 7.73 (m, 2H), 7.63 (d, J=8 Hz, 1H), 7.53-7.48 (m, 1H), 7.30-7.28 (m, 3H), 7.22 (t, J=5.20 Hz, 1H), 6.04 (s, 1H), 3.93 (s, 3H), 2.14 (s, 3H).

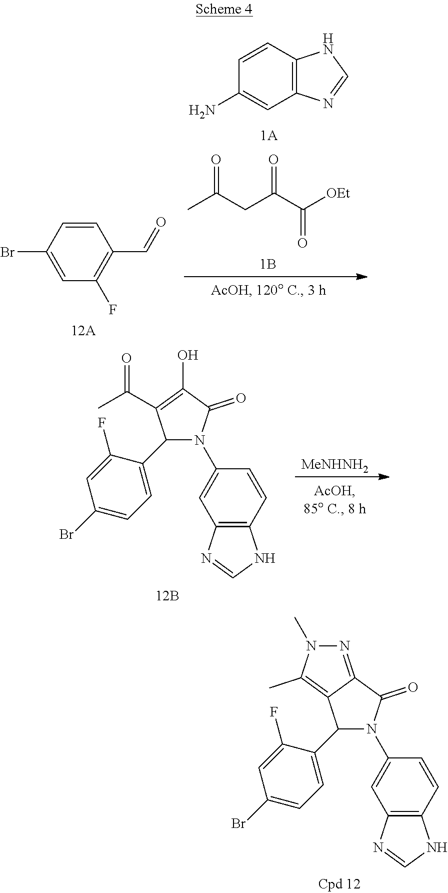

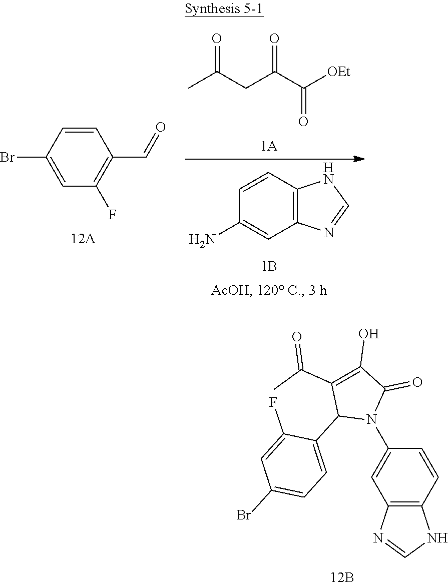 [SMILES: CCOC(=O)C(=O)CC(C)=O ; O=Cc1ccc(Br)cc1F ; Nc1ccc2[nH]cnc2c1 ; CC(=O)C1=C(O)C(=O)N(c2ccc3[nH]cnc3c2)C1c1ccc(Br)cc1F]

A mixture of 12A (500 mg, 2.46 mmol, 1.00 eq), 1B (418 mg, 2.46 mmol, 1.00 eq, HCl) and 1A (389 mg, 2.46 mmol, 348 μL, 1.00 eq) in AcOH (5 mL) was stirred at 120° C. for 2 hours. LCMS showed 12A was consumed completely and one main peak with the desired mass was detected. The mixture was poured into MeCN (5 mL). The mixture was filtered and the filter cake was washed with MeCN (2.5 mL×2). The residue was dried under reduced pressure to give 12B (500 mg, crude) as a dark brown solid, which was used the next reaction without further purification.

LCMS: product RT=0.61 min. m/z=430.0 (M+1)⁺.

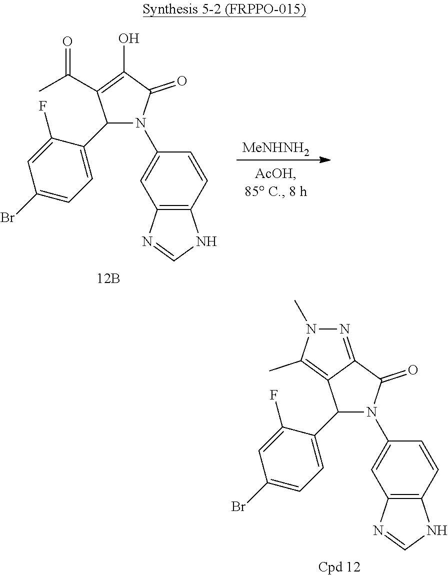 [SMILES: "MeNHNH2" ; CC(=O)C1=C(O)C(=O)N(c2ccc3[nH]cnc3c2)C1c1ccc(Br)cc1F ; Cc1c2c(nn1C)C(=O)N(c1ccc3[nH]cnc3c1)C2c1ccc(Br)cc1F]

To a mixture of 12B (480 mg, 982 μmol, 1.00 eq) in AcOH (5 mL) was added methylhydrazine (410 mg, 3.56 mmol, 469 μL, 3.63 eq). The mixture was stirred at 85° C. for 8 hours. LCMS showed one main peak with the desired mass. The reaction mixture was concentrated under vacuum. The residue was purified by preparative HPLC (column Phenomenex Synergi Max RP 150×50 mm, 10 μm; mobile phase (0.23% formic acid) A—water, B—ACN; gradient 15-45% B, 10 min) to give Cpd 12 (126 mg, 133 μmol, 14% yield, 51.5% purity) as brown solid. The product was repurified by preparative HPLC (column Xtimate C18 150×25 mm, 5 um; mobile phase (0.05% NH₄OH) A—water, B—ACN; gradient 25-55% B, 10 min) to give Cpd 12 (19.8 mg, 44.2 μmol, 15.4% yield, 97.9% purity) as a light yellow solid.

LCMS: product RT=0.88 min, m/z=440.2 (M+1)⁺.

HPLC: RT=1.72 min, 98.0% purity.

¹H NMR: DMSO, 400 MHz: δ 12.5 (s, 1H), 8.19 (s, 1H), 7.71 (s, 1H), 7.49 (d, J=10 Hz, 2H), 7.29-7.21 (m, 3H), 6.62 (s, 1H), 3.87 (s, 3H), 2.13 (s, 3H).

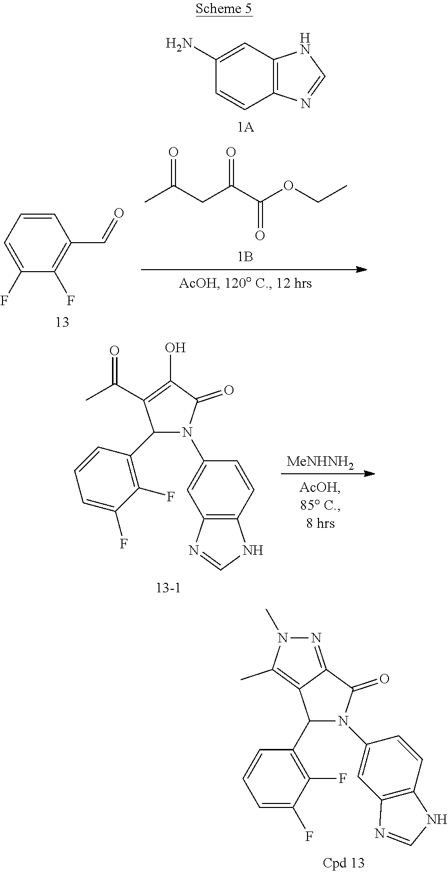

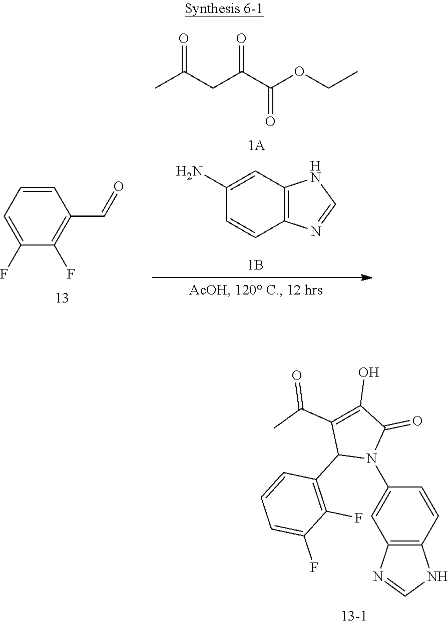 [SMILES: CCOC(=O)C(=O)CC(C)=O ; O=Cc1cccc(F)c1F ; Nc1ccc2nc[nH]c2c1 ; CC(=O)C1=C(O)C(=O)N(c2ccc3[nH]cnc3c2)C1c1cccc(F)c1F]

To a solution of compound 13 (1.00 g, 7.04 mmol, 769 μL, 1.00 eq) in AcOH (20.0 mL) was added compound 1B (HCl salt, 937 mg, 7.04 mmol, 1.00 eq) and compound 1A (1.11 g, 7.04 mmol, 994 μL, 1 eq). The mixture was stirred at 120° C. for 12 hours. LCMS showed the starting material was consumed completely and the desired mass was detected. The reaction solution was poured into water (80.0 mL) and filtered. Then the residue was washed by MeCN (20.0 mL×3) and collected to give compound 13-1 (1.75 g, crude) as a brown solid which was used without further purification.

¹H NMR: DMSO-d₆ 400 MHz: δ 8.33 (s, 1H), 7.75-7.74 (m, J=4 Hz, 1H), 7.57-7.55 (m, 1H), 7.36-7.33 (m, 1H), 7.17-7.14 (m, 1H), 7.11-6.96 (m, 2H), 6.31 (s, 1H), 2.38 (s, 3H).

LCMS: RT=0.882 min, m/z=370 (M+1)⁺.

HPLC: purity: 83.2%.

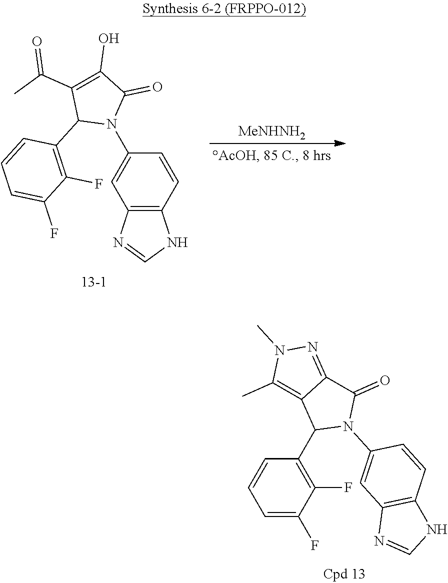 [SMILES: "MeNHNH2" ; CC(=O)C1=C(O)C(=O)N(c2ccc3[nH]cnc3c2)C1c1cccc(F)c1F ; Cc1c2c(nn1C)C(=O)N(c1ccc3[nH]cnc3c1)C2c1cccc(F)c1F]

To a solution of compound 13-1 (0.50 g, 1.35 mmol, 1.00 eq) in AcOH (5.00 mL) was added methylhydrazine (0.39 g, 3.39 mmol, 446 μL, 40.0% purity, 2.50 eq). The mixture was stirred at 85° C. for 8 hours. LCMS showed starting material was consumed completely and the desired mass was detected. The reaction solution was concentrated under reduced pressure to crude product. The crude product was purified by preparative HPLC (column Phenomenex Luna C18 200×40 mm, 10 μm; mobile phase (0.1% formic acid) A—water, B—ACN; gradient 7-37% B, 10 min) to give Cpd 13 (101 mg, 237 μmol, 18% yield, FA) as a brown solid (101 mg, 237 μmol, 17.5% yield, FA) which was repurified by preparative HPLC (column Waters Xbridge 150×50 mm, 10 μm; mobile phase (0.05% NH₄OH) A—water, B—ACN; gradient 12-42% B, 11.5 min) to give Cpd 13 (11.9 mg, 31.4 μmol, 13% yield) as yellow solid.

LCMS: RT=0.894 min, m/z=380 (M+1)⁺.

HPLC: purity: 99.2%.

¹H NMR: MeOD 400 MHz: δ 8.15 (s, 1H), 7.69 (s, 1H), 7.57-7.57 (d, J=8 Hz, 1H), 7.36-7.33 (m, 1H), 7.16-6.95 (m, 3H), 6.58 (s, 1H), 3.93 (s, 3H), 2.19 (s, 3H).

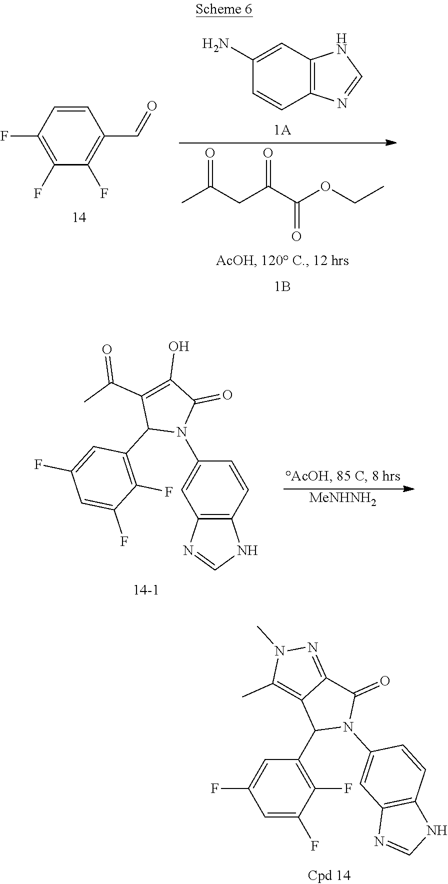

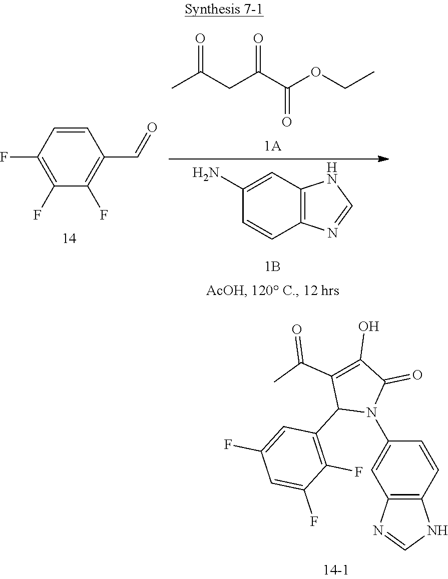

To a solution of compound 14 (1.00 g, 6.25 mmol, 709 μL, 1.00 eq) in AcOH (20.0 mL) was added compound 1B (832 mg, 4.90 mmol, 0.79 eq, HCl) and compound 1A (988 mg, 6.25 mmol, 882 μL, 1.00 eq). The mixture was stirred at 120° C. for 12 hours. LCMS showed the starting material was consumed completely and the desired mass was detected. The reaction solution was poured into water (80 mL) and filtered. Then the residue was washed by MeCN (20 mL×3) and collected to give compound 14-1 (1.40 g, crude) as a brown solid which was used without further purification.

LCMS: RT=0.877 min, m/z=388 (M+1)⁺.

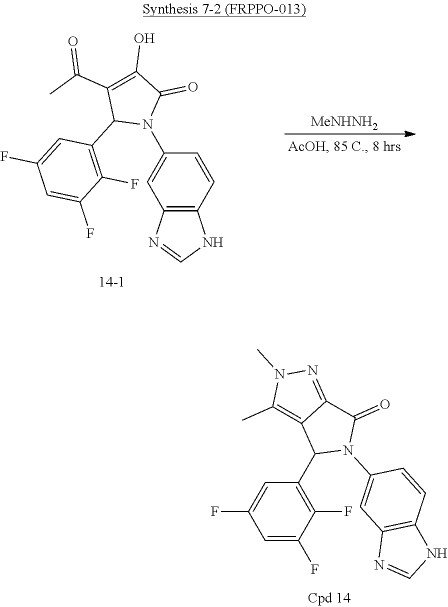 [SMILES: "MeNHNH2" ; CC(=O)C1=C(O)C(=O)N(c2ccc3[nH]cnc3c2)C1c1cc(F)cc(F)c1F ; Cc1c2c(nn1C)C(=O)N(c1ccc3[nH]cnc3c1)C2c1cc(F)cc(F)c1F]

To a solution of compound 14-1 (0.50 g, 1.29 mmol, 1.00 eq) in AcOH (5.00 mL) was added methylhydrazine (340 mg, 2.95 mmol, 389 μL, 40.0% purity, 2.29 eq). The mixture was stirred at 85° C. for 8 hours. LCMS showed starting material was consumed completely and the desired mass was detected. The reaction solution was concentrated under reduced pressure to give crude product. The crude product was purified by preparative HPLC (column Phenomenex Synergi Max RP 150×50 mm, 10 μm; mobile phase (0.26% formic acid) A—water, B—ACN; gradient 10-40% B, 10 min) to give Cpd 14 (102 mg, 228 μmol, 17.6% yield, 99.9% purity, FA) as a yellow solid.

Cpd 14 (102 mg, 228 μmol, 17.6% yield, 99.9% purity, FA) was repurified by preparative HPLC (column Waters Xbridge 150×50 mm, 10 μm; mobile phase (0.05% NH₄OH) A—water, B—ACN; gradient 15-45% B, 11.5 min) to give Cpd 14 (38.5 mg, 96.9 μmol, 42.1% yield) as white solid.

LCMS: RT=0.900 min, m/z=398 (M+1)⁺.

HPLC: purity: 97.6%.

¹H NMR: MeOD 400 MHz: δ 8.17 (s, 1H), 7.70 (s, 1H), 7.60-7.58 (d, J=8 Hz, 1H), 7.37-7.35 (d, J=8 Hz, 1H), 7.04-7.03 (m, 1H), 6.83-6.80 (m, 1H), 6.60 (s, 1H), 3.94 (s, 3H), 2.22 (s, 3H).

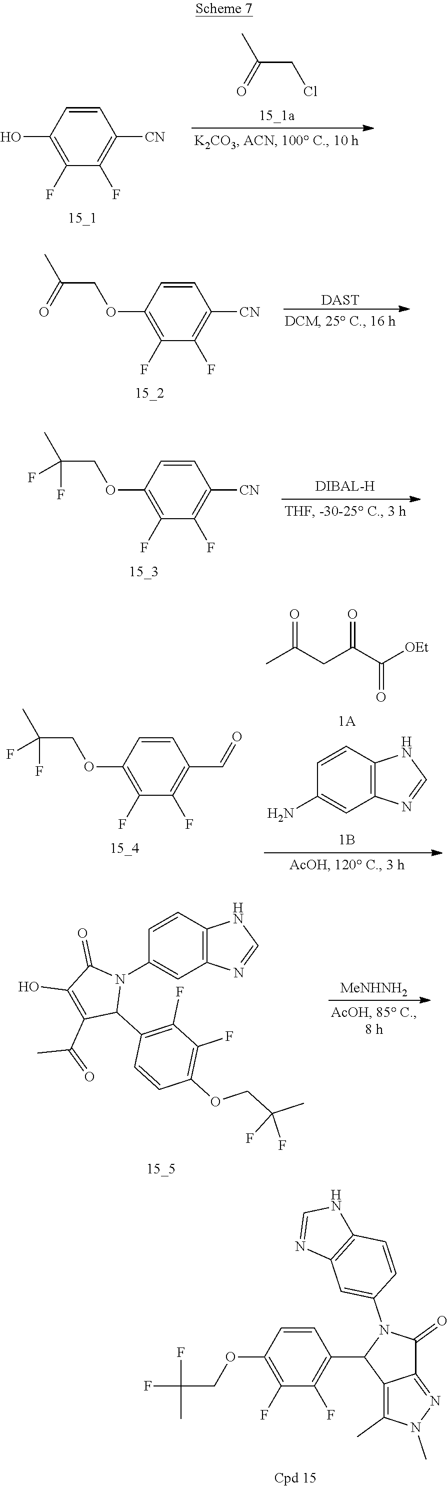

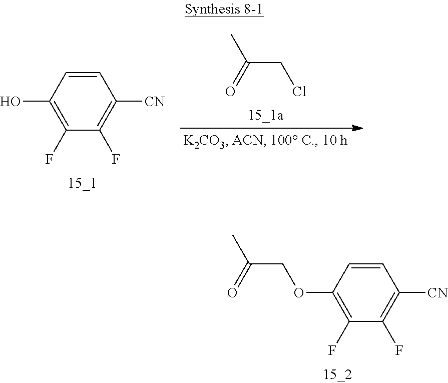

A mixture of compound 15_1, compound 15_1a and K₂CO₃ (8.91 g, 64.5 mmol, 2.00 eq) in ACN (25 mL) was stirred at 100° C. for 10 hours. LCMS showed the desired mass. The reaction mixture was filtered, and the cake was washed with ethyl acetate (40 mL×3), and then the filtrate was added to water (50 mL), then extracted with ethyl acetate (50 mL×3), combined the organic layer and dried over with Na₂SO₄, and then concentrated to give a residue which was purified by preparative HPLC (HCl condition) to give compound 15_2 (2.28 g, 9.58 mmol, 29.7% yield, 88.7% purity) as an off-white solid.

LCMS: RT=0.68 min, m/z=212.1 (M+H)⁺.

¹H NMR: CDCl₃, 400 MHz: δ 7.37-7.32 (m, 1H), 6.76-6.47 (m, 1H), 4.74 (s, 2H), 2.26 (s, 3H).

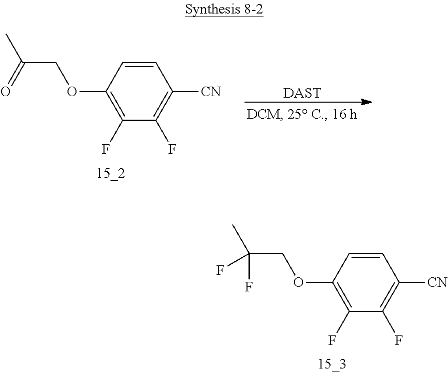

To a solution of compound 15_2 (2.00 g, 9.47 mmol, 1.00 eq) in DCM (10 mL) was added DAST (3.06 g, 19.0 mmol, 2.51 mL, 2.00 eq). The mixture was stirred at 25° C. for 16 hours. TLC (Petroleum ether:Ethyl acetate=10:1) indicated material was consumed completely and one new spot formed. The mixture was poured onto ice water (30 mL) and extracted with ethyl acetate (2×10 mL). The combined organic layers were dried (Na₂SO₄), filtered and concentrated under reduced pressure. The residue was purified by column chromatography (SiO₂, petroleum ether:ethyl acetate=5:1) and concentrated to give compound 15_3 (2.10 g, 9.01 mmol, 95.1% yield) as a yellow oil.

¹H NMR: CDCl₃, 400 MHz: δ 7.39-7.34 (m, 1H), 6.88-6.85 (m, 1H), 4.28 (t, J=6.8 Hz, 2H), 1.79 (t, J=12.0 Hz, 3H).

FNMR: CDCl₃, 400 MHz: δ −154.5, −129.1, −98.1.

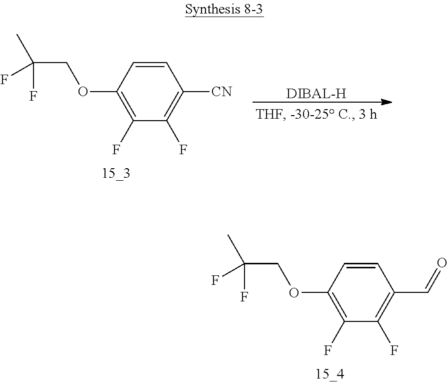 [SMILES: CC(F)(F)COc1ccc(C#N)c(F)c1F ; "DIBAL-H" ; CC(F)(F)COc1ccc(C=O)c(F)c1F]

DIBAL-H (1 M, 17.1 mL, 2.00 eq) was added drop-wise to a solution of compound 15_3 (2.00 g, 8.58 mmol, 1.00 eq) in THF (40 mL) at −30° C., and the mixture was stirred at 25° C. for 3 hours. TLC (petroleum ether:ethyl acetate=5:1, starting material R_{f}=0.45; product R_{f}=0.50) showed the starting material was consumed and LCMS showed the desired mass. The reaction mixture was quenched with NH₄Cl (12.00 mL) at 10° C. and Na₂SO₄ (12.00 g) was added. The mixture was stirred at 10° C. for 5 min, filtered and concentrated under reduce pressure to give a residue which was purified by column chromatography (SiO₂, petroleum ether:ethyl acetate=1:0 to 0:1) and concentrated under reduce pressure to give compound 15_4 as a yellow liquid. TLC (petroleum ether:ethyl acetate=5:1, R_{f}=0.50).

LCMS (crude): RT=0.91 min, m/z=236.9 (M+H)⁺.

¹H NMR: CDCl₃, 400 MHz: δ 10.36 (s, 1H), 7.64 (t, J=2.0 Hz, 1H), 6.90 (t, J=1.6 Hz, 1H), 4.28 (t, J=11.2 Hz, 2H), 1.82 (t, J=18.8 Hz, 3H).

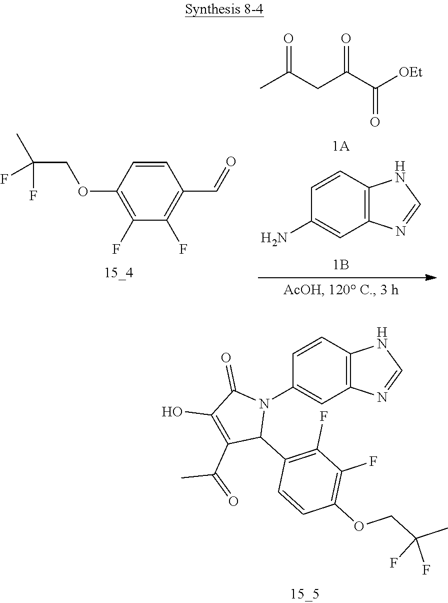 [SMILES: CCOC(=O)C(=O)CC(C)=O ; CC(F)(F)COc1ccc(C=O)c(F)c1F ; Nc1ccc2[nH]cnc2c1 ; CC(=O)C1=C(O)C(=O)N(c2ccc3[nH]cnc3c2)C1c1ccc(OCC(C)(F)F)c(F)c1F]

To a solution of compound 15_4 (500.0 mg, 2.12 mmol, 1.00 eq) in AcOH (2.50 mL) was added compound 1B (282 mg, 2.12 mmol, 1.00 eq) and compound 1A (335 mg, 2.12 mmol, 299 μL, 1.00 eq). The mixture was stirred at 120° C. for 3 hours. LCMS showed the desired mass. The mixture was condensed to give the crude product. The crude product was purified by reversed-phase HPLC (0.1% formic acid H₂O/MeCN) to give compound 15_5 (210.0 mg, 453.2 μmol, 21.4% yield) as a yellow solid which was used without further purification.

LCMS (crude): product: RT=0.64 min, m/z=464.1 (M+H)⁺.

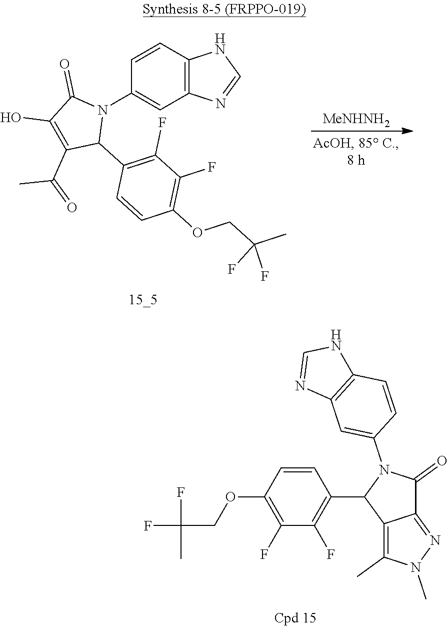 [SMILES: "MeNHNH2" ; CC(=O)C1=C(O)C(=O)N(c2ccc3[nH]cnc3c2)C1c1ccc(OCC(C)(F)F)c(F)c1F ; Cc1c2c(nn1C)C(=O)N(c1ccc3[nH]cnc3c1)C2c1ccc(OCC(C)(F)F)c(F)c1F]

To a solution of compound 15_5 (210 mg, 453 μmol, 1.00 eq) in AcOH (2.50 mL) was added methylhydrazine (140 mg, 3.04 mmol, 160 μL, 6.71 eq). The mixture was stirred at 85° C. for 8 hours. LCMS showed the desired mass. The mixture was concentrated to give the crude product which was purified by preparative HPLC HPLC (column Waters Xbridge 150×50 mm, 10 μm; mobile phase (0.05% NH₄OH) A—water, B—ACN; gradient 22-52% B, 10 min) and lyophilized to give product Cpd 15 (12.95 mg, 27.2 μmol, 6.0% yield, 99.4% purity) as yellow solid.

LCMS: product: RT=0.65 min, m/z=474.1 (M+H)⁺.

HPLC: purity: 99.4%

¹H NMR: DMSO, 400 MHz: δ 12.41 (s, 1H), 8.18 (s, 1H), 7.69 (s, 1H), 7.50 (d, J=8.8 Hz, 1H), 7.29 (d, J=10.8 Hz, 1H), 7.07 (s, 1H), 6.93 (s, 1H), 6.58 (s, 1H), 4.33 (t, J=2.4 Hz, 2H), 3.88 (s, 3H), 2.13 (s, 3H), 1.66 (t, J=19.2 Hz, 3H).

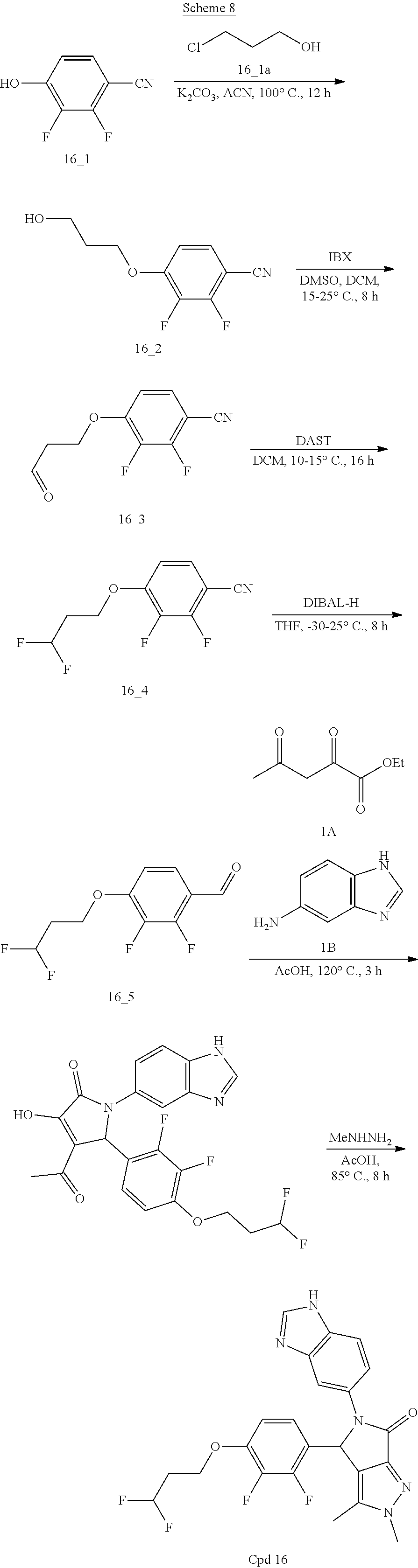

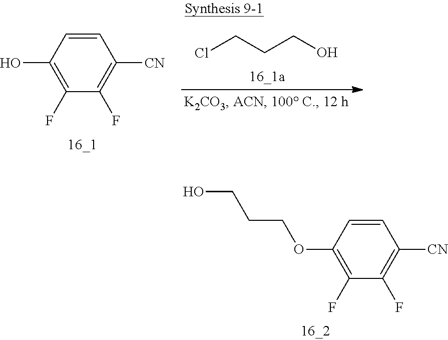 [SMILES: N#Cc1ccc(O)c(F)c1F ; OCCCCl ; N#Cc1ccc(OCCCO)c(F)c1F]

A solution of compound 16_1 (5.00 g, 32.2 mmol, 1.00 eq), compound 16_1a (9.14 g, 96.7 mmol, 8.09 mL, 3.00 eq), K₂CO₃ (8.91 g, 64.5 mmol, 2.00 eq) and KI (535 mg, 3.22 mmol, 0.10 eq) in ACN (25.0 mL) was stirred at 100° C. for 12 hours. LCMS showed the desired mass. The reaction mixture was filtered, and then the filtrate was concentrated to give a residue which was purified by reversed-phase HPLC (0.1% HCl H₂O/ACN) to give compound 16_2 (3.65 g, 15.7 mmol, 48.6% yield, 91.5% purity) as a yellow oil.

LCMS: RT=0.65 min, m/z=214.1 (M+H)⁺.

¹H NMR: CDCl₃, 400 MHz: δ 7.36-7.31 (m, 1H), 6.88-6.84 (m, 1H), 4.28 (t, J=6.4 Hz, 2H), 3.88 (t, J=5.6 Hz, 2H), 2.14-2.04 (m, 2H).

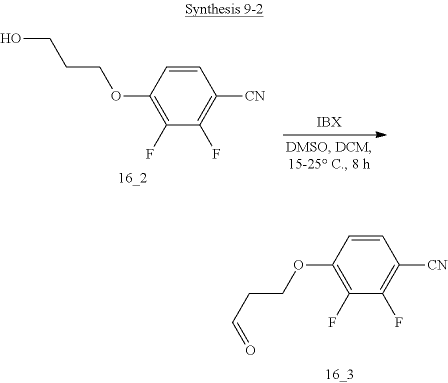 [SMILES: N#Cc1ccc(OCCCO)c(F)c1F ; N#Cc1ccc(OCCC=O)c(F)c1F]

To a solution of compound 16_2 (3.00 g, 14.0 mmol, 1.00 eq) in DCM (20.0 mL) and DMSO (20.0 mL) was added IBX (31.5 g, 112 mmol, 8.00 eq) at 15° C. The mixture was stirred at 25° C. for 8 hours. TLC (petroleum ether:ethyl acetate=1:1, starting material-R_{f}=0.25, product-R_{f}=0.45) indicated material was consumed completely and a new spot formed. The reaction mixture was filtered and washed with DCM (50 mL). The combined filtrate washed with water (100×2 mL) and saturated sodium thiosulfate aqueous solution (100 mL). The organic solution was washed with saturated NaCl (40 mL), dried over Na₂SO₄, filtered and concentrated under reduced pressure to give compound 16_3 (2.50 g, crude) as a yellow oil.

¹H NMR: CDCl₃, 400 MHz: δ 9.81 (s, 1H), 7.34-7.30 (m, 1H), 6.89-6.85 (m, 1H), 4.41 (t, J=5.6 Hz, 2H), 3.01 (t, J=6.0 Hz, 2H).

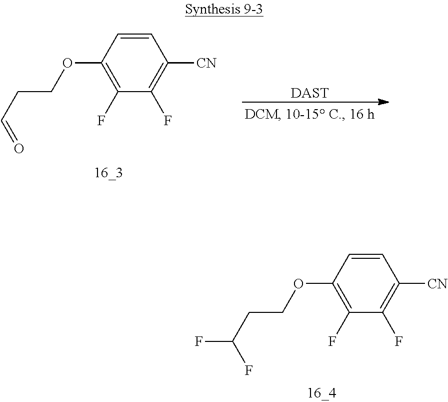 [SMILES: N#Cc1ccc(OCCC=O)c(F)c1F ; "DAST" ; N#Cc1ccc(OCCC(F)F)c(F)c1F]

A solution of compound 16_3 (1.80 g, 8.52 mmol, 1.00 eq) in DCM (18.0 mL) added DAST (3.29 g, 20.4 mmol, 2.70 mL, 2.40 eq) to the solution at 10-15° C. and stirred for 16 hours. TLC (petroleum ether:ethyl acetate=2:1, material-R_{f}=0.10, product-R_{f}=0.60) showed a new spot formed. The solution mixture was poured into ice water (10.00 mL). The mixture was extracted with ethyl acetate (10 mL×3), dried by Na₂SO₄, filtered and concentrated under reduce pressure to give a residue which was purified by column chromatography (SiO₂, petroleum ether:ethyl acetate=1:0 to 0:1) and concentrated under reduce pressure to give compound 16_4 (1.70 g, 7.29 mmol, 85.5% yield) as a yellow solid.

¹H NMR: CDCl₃, 400 MHz: δ 7.39-7.36 (m, 1H), 6.85-6.83 (m, 1H), 6.25-6.00 (m, 1H), 4.30 (t, J=6.0 Hz, 2H), 2.47-2.38 (m, 2H).

FNMR: CDCl₃, 400 MHz: δ −155.3, -129.5, -118.4.

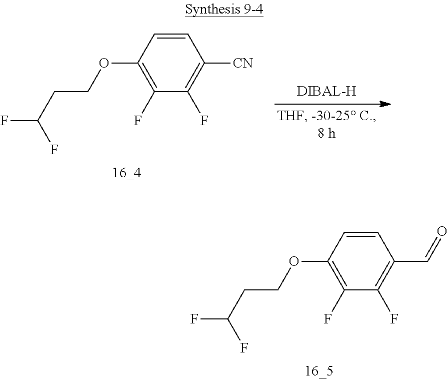 [SMILES: N#Cc1ccc(OCCC(F)F)c(F)c1F ; O=Cc1ccc(OCCC(F)F)c(F)c1F]

DIBAL-H (1.00 M in THF, 9.44 mL, 2.00 eq) was added drop-wise to a solution of compound 16_4 (1.10 g, 4.72 mmol, 1.00 eq) in THF (25 mL) at −30° C., and the mixture was stirred at 25° C. for 3 hours. TLC (petroleum ether:ethyl acetate=3:1, starting material-R_{f}=0.64, product-R_{f}=0.68) showed remaining starting material and a new spot was formed. DIBAL-H (1.00 M in THF, 3.30 mL, 0.70 eq) was added to the mixture at -30° C., the mixture was stirred at 25° C. for 5 hours. The reaction mixture was quenched with NH₄Cl (12 . mL) and Na₂SO₄ (12 g) added, the mixture was stirred at 25° C. for 10 min, filtered to remove the filtrate and concentrated under reduce pressure to give a residue which was purified by reversed-phase HPLC (0.1% formic acid H₂O/ACN) and concentrated under reduce pressure to give compound 16_5 (0.30 g, 1.07 mmol, 22.7% yield, 84.2% purity) as a yellow oil.

LCMS: product: RT=0.85 min, m/z=236.9 (M+H)⁺.

¹H NMR: CDCl₃, 400 MHz: δ 10.21 (s, 1H), 7.64 (t, J=7.2 Hz, 1H), 6.86 (t, J=16.0 Hz, 1H), 6.26-5.97 (m, 1H), 4.32 (t, J=6.0 Hz, 2H), 2.85-2.40 (m, 2H).

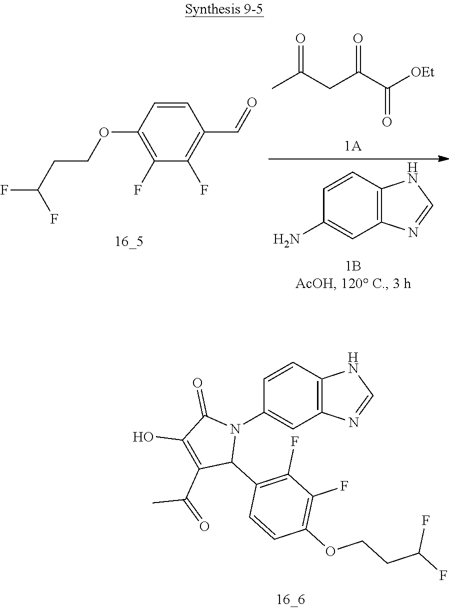

To a solution of compound 16_5 (200 mg, 846 μmol, 1.00 eq) in AcOH (1.00 mL) was added compound 1B (112 mg, 846 μmol, 1.00 eq) and compound 1A (133 mg, 846 μmol, 119 μL, 1.00 eq). The mixture was stirred at 120° C. for 3 hours. LCMS showed the desired mass. The mixture was concentrated under reduce pressure to give a crude product. The crude product was purified by preparative HPLC (column Phenomenex Luna C18 200×40 mm, 10 μm; mobile phase (0.1% TFA) A—water, B—ACN; gradient 13-43% B, 10 min) to give compound 16_6 (150 mg, 323 μmol, 38.2% yield) as a yellow solid.

LCMS (crude): product: RT=0.74 min, m/z=464.0 (M+H)⁺.

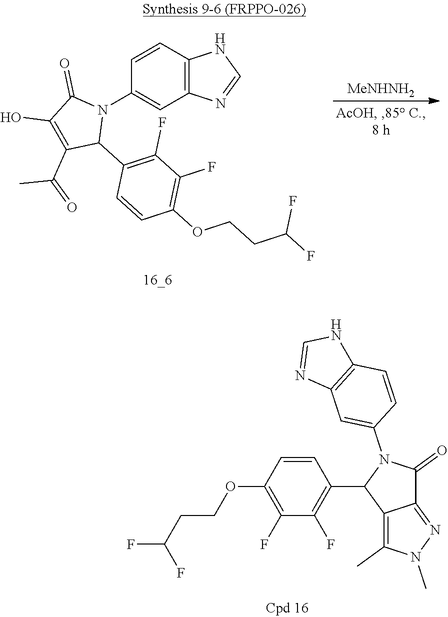 [SMILES: "MeNHNH2" ; CC(=O)C1=C(O)C(=O)N(c2ccc3[nH]cnc3c2)C1c1ccc(OCCC(F)F)c(F)c1F ; Cc1c2c(nn1C)C(=O)N(c1ccc3[nH]cnc3c1)C2c1ccc(OCCC(F)F)c(F)c1F]

To a solution of compound 16_6 (150.0 mg, 323.7 μmol, 1.00 eq) in AcOH (1.00 mL) was added methylhydrazine (50.00 mg, 1.09 mmol, 57.14 μL, 3.35 eq). The mixture was stirred at 85° C. for 8 hours. The desired mass was detected by LCMS. The mixture was concentrated to give the crude product which was purified by preparative HPLC (column Xtimate C18 150×25 mm, 5 μm; mobile phase (0.05% NH₄OH) A—water, B—ACN; gradient 23-53% B, 10 min) and lyophilized to give Cpd 16 (15.46 mg, 32.30 μmol, 9.98% yield, 98.9% purity) as a light yellow solid.

LCMS (crude): product: RT=0.85 min, m/z=474.1 (M+H)⁺.

LCMS: product: RT=0.62 min. m/z=474.0 (M+H)⁺.

HPLC: purity 98.9%.

¹H NMR: DMSO, 400 MHz: δ 12.44 (d, J=6.4 Hz, 1H), 8.19 (d, J=4.4 Hz, 1H), 7.68 (d, J=9.6 Hz, 1H), 7.56 (dd, J₁=56.8 Hz, J₂=36.4 Hz, 1H), 7.32 (dd, J₁=33.2 Hz, J₂=30.4 Hz, 1H), 7.08-7.02 (m, 1H), 6.92 (t, J=8.4 Hz, 1H), 6.59 (d, J=10.4 Hz, 1H), 6.59-6.00 (m, 1H), 4.14-4.10 (m, 2H), 3.88 (s, 3H), 2.34-2.23 (m, 2H), 2.13 (s, 3H).

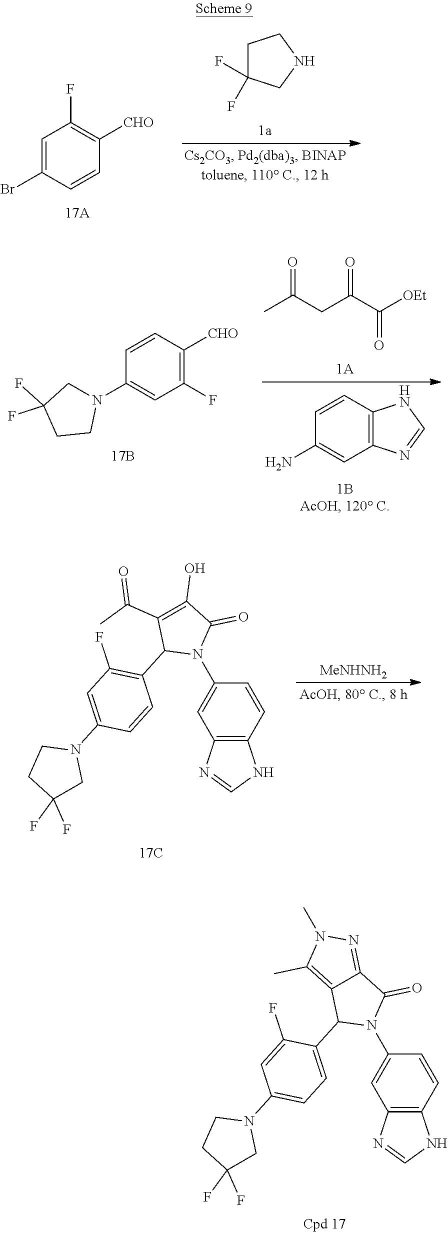

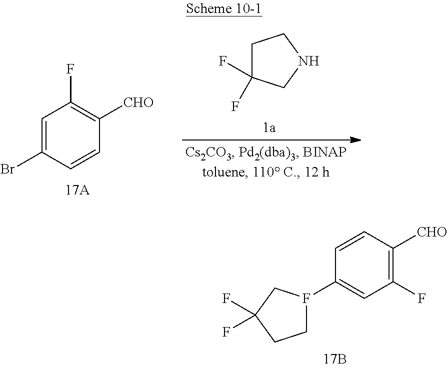

To a mixture of compound 17A (1.00 g, 4.52 mmol, 1.00 eq.) and compound 1a (820 mg, 5.43 mmol, 1.20 eq., HCl) in toluene (10 mL) Cs₂CO₃ (5.90 g, 18.1 mmol, 4.00 eq.) was added BINAP (563 mg, 904 μmol, 0.20 eq.) and Pd₂(dba)₃ (414 mg, 452 μmol, 0.10 eq.) in one portion under N₂. The mixture was stirred at 110° C. for 12 hours under N₂. LCMS showed compound 17A was consumed completely and one peak with the desired mass was detected. The reaction mixture was filtered and then water (50 mL) added and extracted with ethyl acetate (50 mL×3). The combined organic layers were washed with brine (50 mL), dried over Na₂SO₄, filtered and concentrated under reduced pressure to give a residue which was purified by column chromatography (SiO₂, petroleum ether/ethyl acetate=1:0 to 0:1, TLC, petroleum ether/ethyl acetate=3:1, R_{f}=0.40) to give a crude product. The crude product was purified by preparative HPLC (column Phenomenex Synergi Max RP 150×50 mm, 10 μm; mobile phase (0.23% formic acid) A—water, B—ACN; gradient 28-58% B, 10 min). Compound 17B (250 mg, 1.03 mmol, 22.7% yield, 97.0% purity) was obtained as a yellow solid.

LCMS: RT=0.856 min, m/z=237.2 (M+1)⁺.

HPLC: purity=97.0%.

¹H NMR: 400 MHz CDCl₃: δ 10.12 (s, 1H), 7.92 (d, J=8.8 Hz, 1H), 6.82 (d, J=6.4 Hz, 1H), 6.79-6.76 (m, 1H), 3.79 (t, J=12.8 Hz, 2H), 3.69 (t, J=7.2 Hz, 2H), 2.65-2.54 (m, 2H).

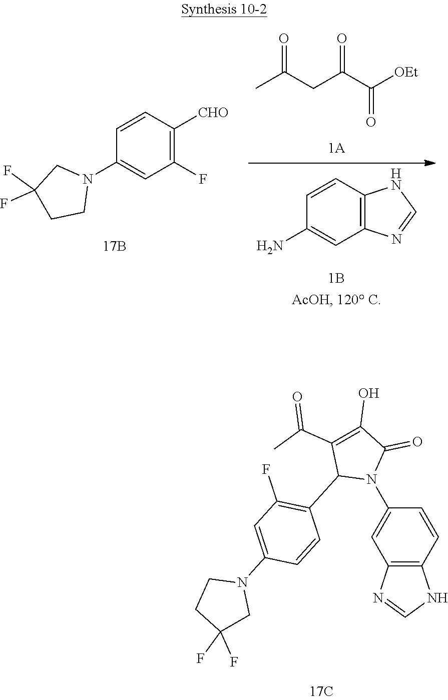 [SMILES: O=Cc1ccc(N2CCC(F)(F)C2)cc1F ; CCOC(=O)C(=O)CC(C)=O ; Nc1ccc2[nH]cnc2c1 ; "AcOH, 120° C." ; CC(=O)C1=C(O)C(=O)N(c2ccc3[nH]cnc3c2)C1c1ccc(N2CCC(F)(F)C2)cc1F]

To a solution of compound 17B (0.20 g, 872 μmol, 1.00 eq.) in AcOH (3.0 mL), 1B (148 mg, 872 μmol, 1.00 eq., HCl) and 1A (138 mg, 872 μmol, 123 μL, 1.00 eq.) were added and the mixture was heated to 120° C. and stirred for 2 hours. TLC (petroleum ether:ethyl acetate=3:1, reactant R_{f}=0.5) showed compound 17B was consumed completely and LCMS showed the desired mass. The mixture was cooled to 25° C. and concentrated in vacuum to give a residue which was purified by preparative HPLC (column Phenomenex Synergi Max RP 150×50 mm, 10 μm; mobile phase (0.23% formic acid) A—water, B—ACN; gradient 13-43% B, 11 min) to give compound 17C (0.08 g, 76.6 μmol, 8.79% yield, 43.7% purity) as a dark brown solid.

LCMS: RT=0.855 min, m/z=392 (M+1)⁺.

HPLC: RT=0.955 min, 11.2% purity.

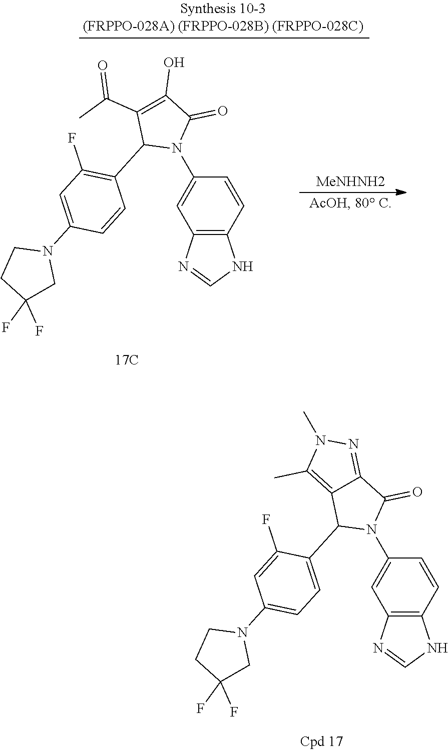 [SMILES: CC(=O)C1=C(O)C(=O)N(c2ccc3[nH]cnc3c2)C1c1ccc(N2CCC(F)(F)C2)cc1F ; "AcOH, 80° C." ; "MeNHNH2" ; Cc1c2c(nn1C)C(=O)N(c1ccc3[nH]cnc3c1)C2c1ccc(N2CCC(F)(F)C2)cc1F]

To a solution of compound 17C (0.08 g, 175 μmol, 1.00 eq) in AcOH (1.00 mL) AcOH (1.00 mL), methylhydrazine (0.31 g, 6.73 mmol, 354 μL, 38.4 eq) was added and the mixture was heated to 85° C. for 8 hours. The mixture was cooled to 25° C. and concentrated to give a residue which was purified by preparative HPLC (column Kromasil 150×25 mm, 10 μm; mobile phase (0.1% TFA) A—water, B—ACN; gradient 25-55% B, 10 min) to give a the product, which was repurified by preparative HPLC (column Waters Xbridge 150×50 mm, 10 μm; mobile phase (0.05% NH₄OH) A—water, B—ACN; gradient 20-50% B, 10 min) to give Cpd 17 (0.01 g, 15.8 μmol, 9.02% yield, 73.8% purity) as an off-white solid. Cpd 17 was purified again by HPLC (column Waters Xbridge 150×50 mm, 10 μm; mobile phase (0.05% NH₄OH) A—water, B—ACN; gradient 22-52% B, 11.5 min) to give Cpd 17 (0.17 mg, 0.36 μmol, 1.70% yield, 100% purity) as a white solid.

LCMS: RT=0.860 min, m/z=467.3 (M+1)⁺.

HPLC: RT=2.446 min, 100% purity.

¹H NMR: 400 MHz, CDCl₃: δ 7.94-7.97 (m, 1H) 7.68-7.85 (m, 1H) 7.43-7.59 (m, 1H) 7.13-7.22 (m, 1H) 6.81-6.89 (m, 1H) 6.31-6.34 (m, 1H) 6.13 (s, 1H) 6.09-6.12 (m, 1H) 3.92-3.94 (s, 3H) 3.51-3.60 (m, 2H) 3.39-3.44 (m, 2H) 2.36-2.49 (m, 2H) 2.16-2.19 (s, 3H).

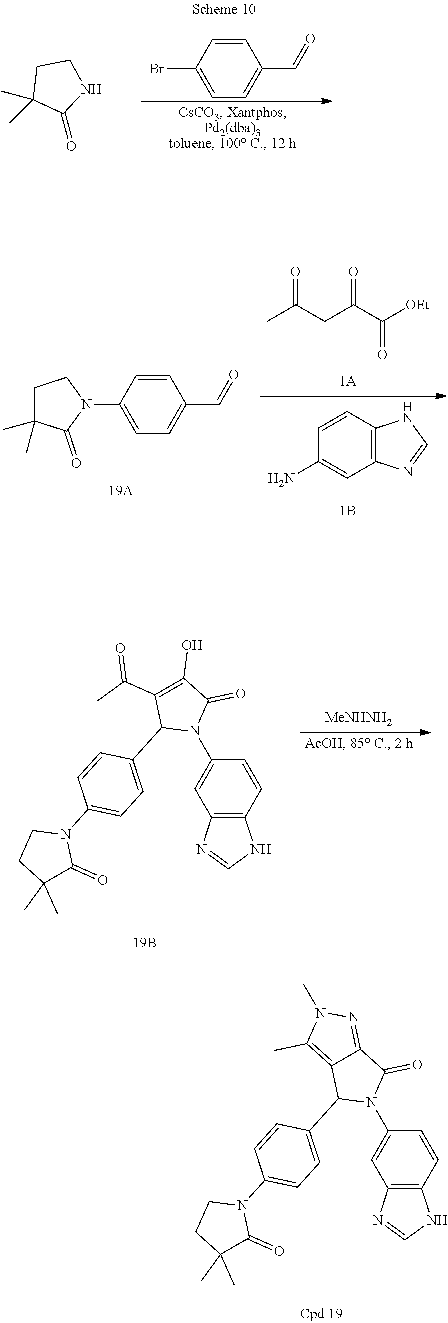

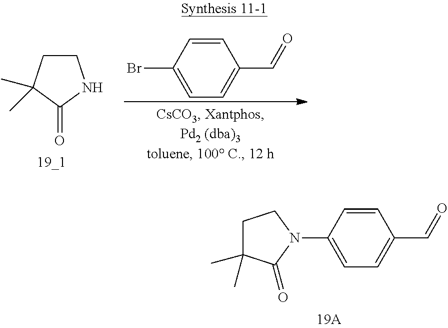

A mixture of compound 1 (500 mg, 2.57 mmol, 1.00 eq), compound 19 (348 mg, 3.08 mmol, 1.20 eq), Pd₂(dba)₃ (117 mg, 128 μmol, 0.05 eq), XantPhos (148 mg, 256 μmol, 0.10 eq) and Cs₂CO₃ (2.51 g, 7.70 mmol, 3.00 eq) in toluene (5 mL) was degassed and purged with N₂ 3 times, and then the mixture was stirred at 100° C. for 12 hours under N₂ atmosphere. LCMS showed compound 1 was consumed completely and one main peak with the desired mass was detected. TLC (petroleum ether:ethyl acetate=2:1) indicated compound 1 was consumed completely and two new spots formed. The reaction mixture was concentrated under reduced pressure to remove toluene. The crude product was purified by flash silica gel chromatography (ISCO; 20 g SepaFlash Silica Flash Column, Eluent of 0-20% ethyl acetate/petroleum ether gradient 15 mL/min). Compound 19A (500 mg, 2.17 mmol, 84.4% yield, 94.1% purity) was obtained as a white solid.

LCMS: RT=0.686 min, m/z=218.1 (M+H)⁺.

¹H NMR: 400 MHz, CDCl₃: δ 9.96 (s, 1H), 7.93-7.87 (m, 4H), 3.87-3.78 (t, J=6.8 Hz, 2H), 2.11-2.00 (t, J=7.2 Hz, 2H), 1.29-1.25 (s, 6H).

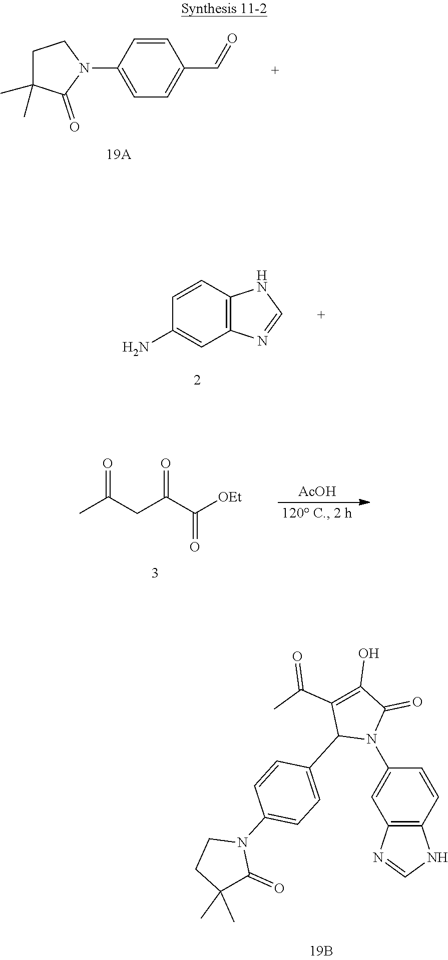

To a mixture of compound 19A (400 mg, 1.84 mmol, 1.00 eq) in AcOH (5 mL) was added compound 2 (245 mg, 1.84 mmol, 1.00 eq) and ethyl ester 3 (291 mg, 1.84 mmol, 259 μL, 1.00 eq). The mixture was stirred at 120° C. for 2 hours. LCMS showed compound 19A was consumed and one main peak with the desired mass was detected. The mixture was concentrated in vacuum to remove AcOH. The mixture was purified by reversed-phase HPLC (0.1% formic acid H₂O/MeCN) to give compound 19B (0.2 g, 449 μmol, 24.4% yield) as a brown solid.

LCMS: RT=0.628 min, m/z=445.11 (M+H)⁺.

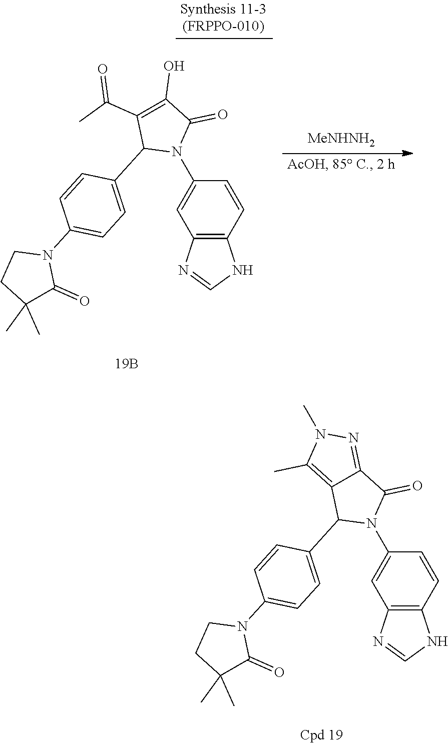 [SMILES: "MeNHNH2" ; CC(=O)C1=C(O)C(=O)N(c2ccc3[nH]cnc3c2)C1c1ccc(N2CCC(C)(C)C2=O)cc1 ; "AcOH, 85° C., 2 h" ; Cc1c2c(nn1C)C(=O)N(c1ccc3[nH]cnc3c1)C2c1ccc(N2CCC(C)(C)C2=O)cc1]

To a mixture of compound 19B (200 mg, 449 μmol, 1.00 eq) in AcOH (2 mL) was added methylhydrazine (41.4 mg, 899 μmol, 47.3 μL, 2.00 eq). The mixture was stirred at 85° C. for 2 hours. LCMS (RT=0.614 min) showed compound 19B was consumed and one main peak with the desired mass was detected. The mixture was concentrated in vacuum. The crude product was purified by flash column chromatography followed by HPLC HPLC (column Shim-pack C18 150×25, 10 μm; mobile phase (0.23% formic acid) A—water, B—ACN; gradient 9-39% B, 10 min) to give Cpd 19 (150 mg). Cpd 19 was repurified by column chromatography followed by preparative HPLC (column Xtimate C18 150×25 mm, 5 μm; mobile phase (0.05% NH₄OH) A—water, B—ACN; gradient 21-51% B, 10 min) to give Cpd 19 (50.0 mg, 110 μmol, 24.4% yield, 97% purity) as a white solid.

LCMS: RT=0.872 min, m/z=455.4 (M+H)⁺.

HPLC: RT=1.874 min, purity: 97.3%

¹H NMR: 400 MHz, CDCl₃: δ 7.95 (s, 1H), 7.85-7.74 (m, 1H), 7.69-7.57 (m, 1H), 7.57-7.55 (d, J=8 Hz, 2H), 7.25 (m, 1H), 7.19-7.17 (d, J=8.4 Hz, 2H), 5.99 (s, 1H), 3.94 (s, 3H), 3.78-3.77 (m, 2H), 2.13 (s, 3H), 1.98-1.95 (m, 2H), 1.22 (s, 6H).

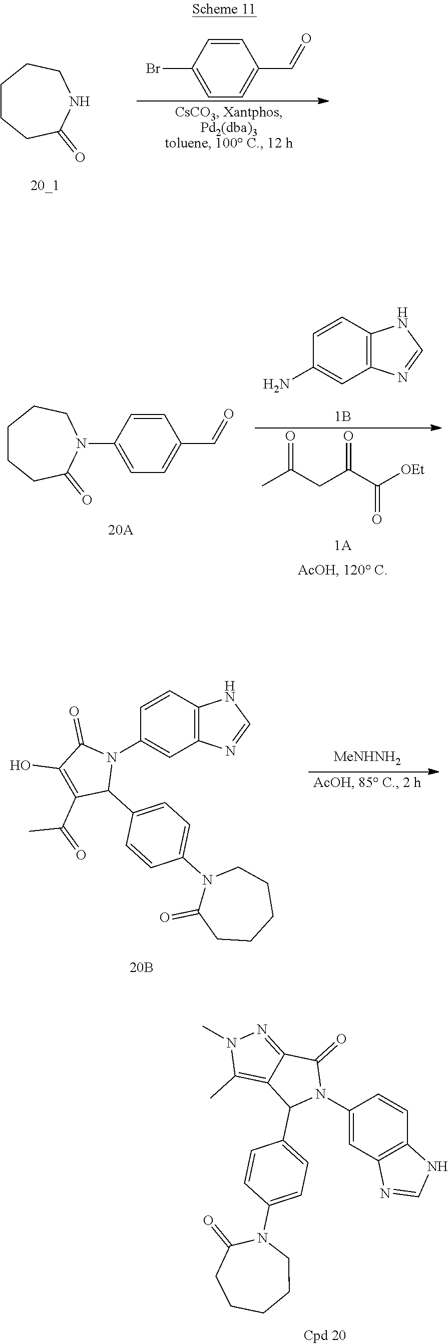

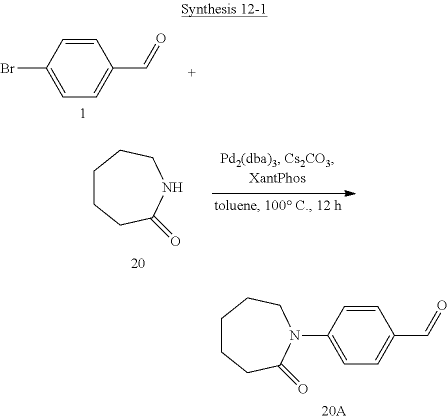 [SMILES: O=Cc1ccc(Br)cc1 ; O=C1CCCCCN1 ; O=Cc1ccc(N2CCCCCC2=O)cc1]

A mixture of compound 1 (3 g, 16.2 mmol, 1.00 eq), compound 20 (2.20 g, 19.4 mmol, 1.20 eq), Pd₂(dba)₃ (743 mg, 811 μmol, 0.05 eq), XantPhos (938 mg, 1.62 mmol, 0.10 eq) and Cs₂CO₃ (15.8 g, 48.6 mmol, 3.00 eq) in toluene (30.0 mL) was degassed and purged with N₂ 3 times, and then the mixture was stirred at 100° C. for 12 hours under N₂ atmosphere. LCMS showed compound 1 was consumed completely and one main peak with the desired mass was detected. TLC (petroleum ether:ethyl acetate=0:1, starting material: R_{f}=0.78, product: R_{f}=0.46) indicated compound 1 was consumed completely and one new spot formed. The reaction mixture was filtered. The crude product was purified by flash silica gel chromatography (ISCO; 40 g SepaFlash Silica Flash Column, Eluent of 0-20% ethyl acetate/petroleum ether gradient 30 mL/min). Compound 20A (2.05 g, 8.53 mmol, 52.5% yield, 90.3% purity) was obtained as a yellow liquid.

LCMS: RT=0.634 min, m/z=218.1 (M+H)⁺.

¹H NMR: 400 MHz, CDCl₃: δ 9.98 (s, 1H), 7.93-7.86 (m, 2H), 7.46-7.40 (m, 2H), 3.87-3.79 (m, 2H), 2.78-2.69 (m, 2H), 1.91-1.82 (m, 6H).

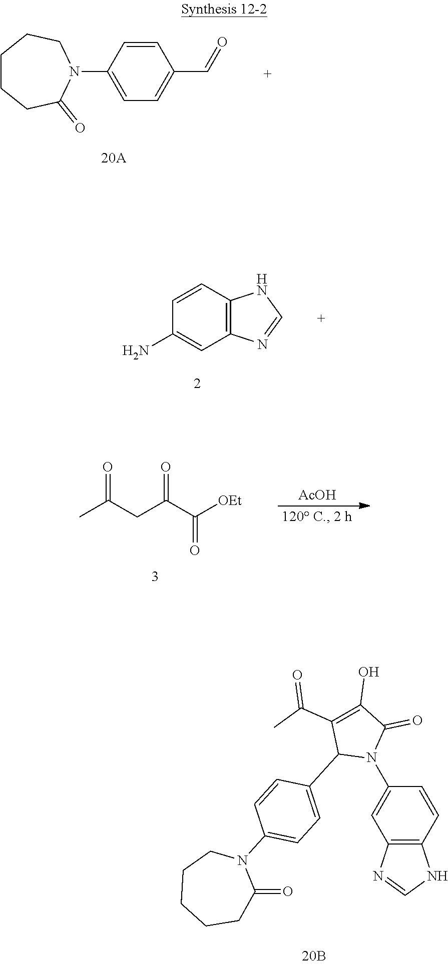 [SMILES: O=Cc1ccc(N2CCCCCC2=O)cc1 ; "AcOH" ; Nc1ccc2[nH]cnc2c1 ; CCOC(=O)C(=O)CC(C)=O ; "120° C., 2 h" ; CC(=O)C1=C(O)C(=O)N(c2ccc3[nH]cnc3c2)C1c1ccc(N2CCCCCC2=O)cc1]

To a mixture of compound 20A (0.50 g, 2.30 mmol, 1.00 eq) in AcOH (5 mL) was added compound 2 (306 mg, 2.30 mmol, 1.00 eq) and compound 3 (363 mg, 2.30 mmol, 324 μL, 1.00 eq). The mixture was stirred at 120° C. for 2 hours. LCMS showed compound 20A was consumed and one main peak with the desired mass was detected. The mixture was concentrated in vacuum to remove AcOH. The mixture was purified by reversed-phase HPLC (0.1% formic acid H₂O/MeCN) to give compound 20B (0.30 g, 674 μmol, 29.3% yield) as yellow solid.

LCMS: RT=0.709 min. m/z=455.2 (M+H)⁺.

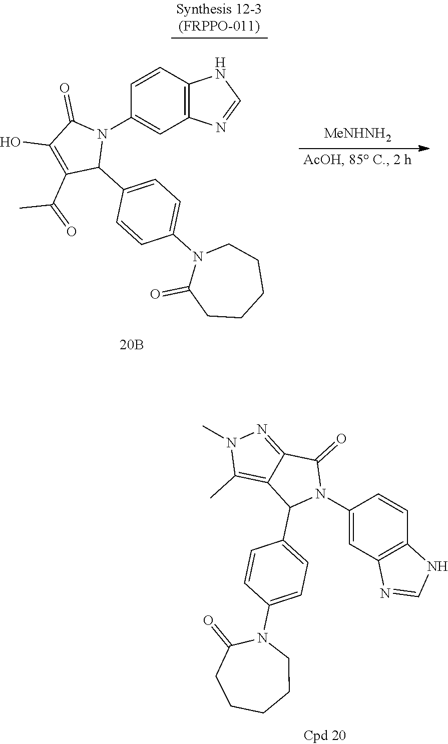 [SMILES: "MeNHNH2" ; CC(=O)C1=C(O)C(=O)N(c2ccc3[nH]cnc3c2)C1c1ccc(N2CCCCCC2=O)cc1 ; "AcOH, 85° C., 2 h" ; Cc1c2c(nn1C)C(=O)N(c1ccc3[nH]cnc3c1)C2c1ccc(N2CCCCCC2=O)cc1]

To a mixture of compound 20B (0.30 g, 674 μmol, 1.00 eq) in AcOH (5 mL) was added methylhydrazine (148 mg, 1.29 mmol, 169 μL, 1.91 eq). The mixture was stirred at 85° C. for 2 hours. LCMS showed compound 20B (RT=0.591 min) was consumed and one main peak with the desired mass was detected. The mixture was concentrated in vacuum. The crude was purified by column chromatography followed by preparative HPLC (column Phenomenex Luna C18 200×40 mm, 10 μm; mobile phase (0.1% formic acid) A—water, B—ACN; gradient 3-33% B, 10 min) to give Cpd 20 (150 mg, 330.02 μmol, 48.90% yield) as a brown solid.

HPLC: RT=1.732 min, purity: 96.3%.

Cpd 20 was further purified by preparative HPLC (column Xtimate C18 150×25 mm, 5 μm; mobile phase (0.05% NH₄OH) A—water, B—ACN; gradient 13-43% B, 10 min) to give Cpd 20 (20.0 mg, 43.6 μmol, 9.91% yield, 99.1% purity) as an off-yellow solid.

LCMS: RT=0.998 min, m/z=455.2 (M+H)⁺; RT=0.848 min, m/z=455.2 (M+H⁺).

HPLC: RT=1.451 min, purity: 87.3%; RT=1.732 min, purity: 99.1%.

¹H NMR: CDCl₃: δ 7.88 (s, 1H), 7.63 (s, 1H), 7.23-7.21 (m, 1H), 7.17 (d, J=8.4 Hz, 3H), 7.10 (d, J=8.4 Hz, 2H), 5.99 (s, 1H), 3.94 (s, 3H), 3.67 (d, J=7.6 Hz, 2H), 2.67 (d, J=6.4 Hz, 2H), 2.15 (a, 3H), 1.79 (s, 6H).

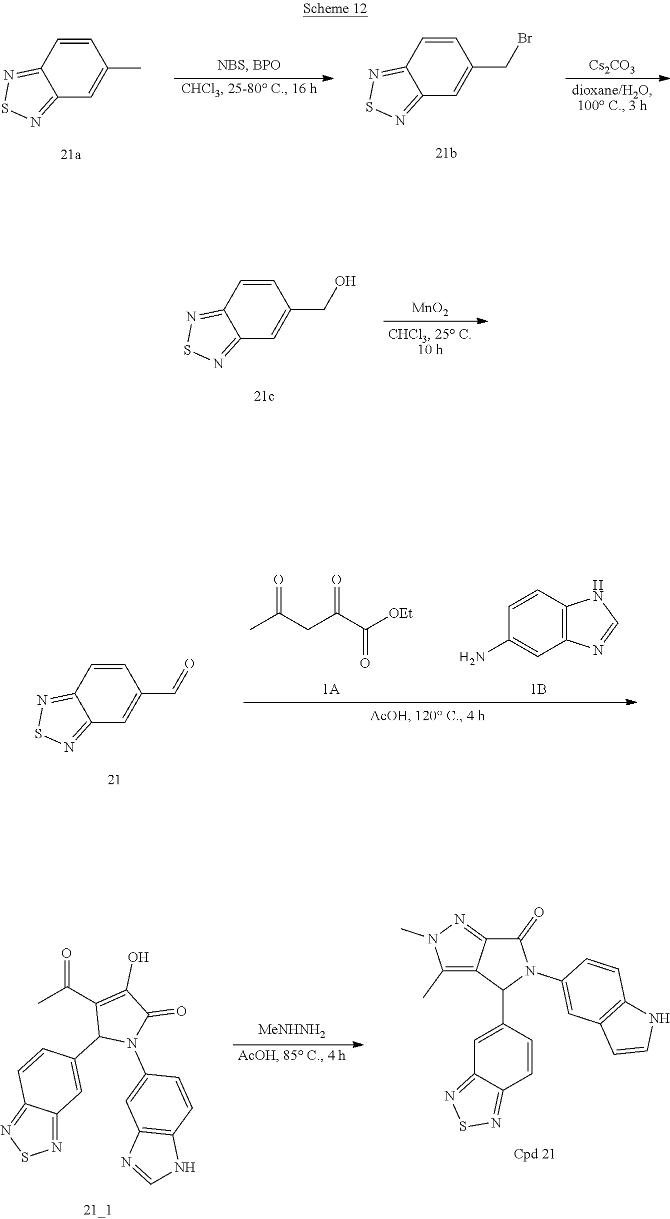

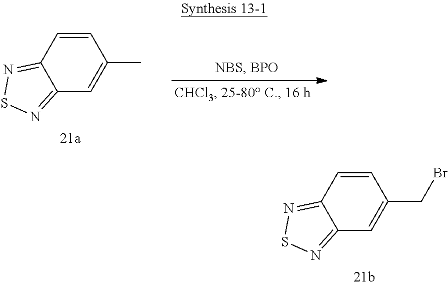

To a solution of compound 21a (4.2 g, 27.9 mmol, 1.00 eq) in CCl₄ (110.0 mL) was added NBS (5.08 g, 28.5 mmol, 1.02 eq) and benzoyl peroxide (677.3 mg, 2.80 mmol, 0.10 eq) at 25° C. The mixture was stirred at 80° C. for 16 hours. LCMS showed compound 21a was consumed and a main peak with the desired mass was formed. The resulting mixture was poured into water (100 mL), then extracted with DCM (100 mL×2). The combined organic layers was dried over Na₂SO₄, filtered, and concentrated under vacuum. The residue was purified by silica gel chromatography (petroleum ether/ethyl acetate=100:1, R_{f}=0.5) to give compound 21b (6 g, 19.9 mmol, 71.2% yield, 76.0% purity) as a light yellow solid.

LCMS: product RT=0.842 min, m/z=230.7 (M+1)⁺.

¹H NMR: 400 MHz, CDCl₃: δ 8.01-7.97 (m, 2H), 7.67-7.63 (m, 1H), 4.67 (s, 1H).

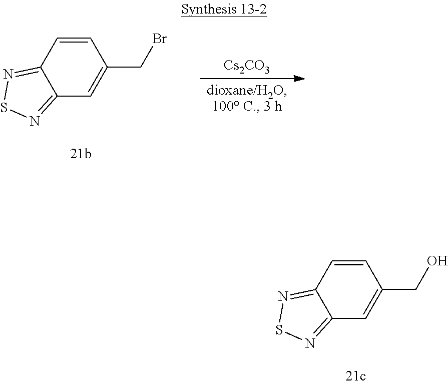

Compound 21b (5.60 g, 18.6 mmol, 1 eq) and Cs₂CO₃ (30.3 g, 92.9 mmol, 5.00 eq) in dioxane (60.0 mL) and H₂O (60.0 mL) were stirred at 100° C. for 3 hours. LCMS showed reactant 21b was consumed and a main peak with the desired mass was formed. The mixture was concentrated under vacuum to give the crude product. The crude product was purified by reversed-phase C18 column chromatography HPLC (0.1% formic acid, 0-25% MeCN in H₂O) to give compound 21c (1.70 g, 10.1 mmol, 54.5% yield, 98.9% purity) as a white solid.

LCMS: product RT=0.457 min, m/z=167.1 (M+1)⁺.

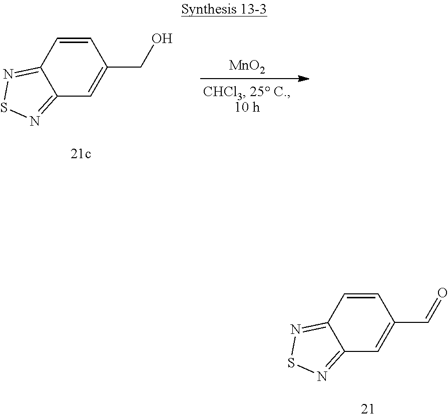 [SMILES: OCc1ccc2nsnc2c1 ; "MnO2" ; "CHCl3, 25° C.,10 h" ; O=Cc1ccc2nsnc2c1]

To a solution of compound 21c (1.70 g, 10.0 mmol, 1.00 eq) in CHCl₃ (87.0 mL) was added MnO₂ (8.71 g, 100.2 mmol, 10.0 eq) at 25° C. The mixture was stirred 10 hours at 25° C. LCMS showed reactant 21c was consumed completely and one main peak with the desired mass was detected. The mixture was filtered and the organic phase was concentrated in vacuum to give compound 21 (1.20 g, crude) as a white solid.

LCMS (crude): product RT=0.684 min; m/z=165.1 (M+1)⁺.

H NMR: 400 MHz CDCl₃: δ 10.23 (s, 1H), 8.52 (t, J=1.2 Hz, 1H), 8.13 (d, J=1.2 Hz, 2H).

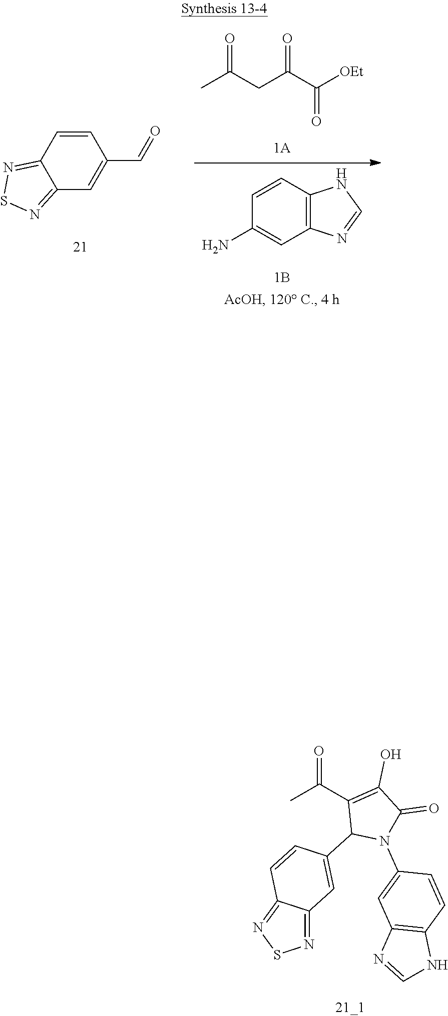 [SMILES: CCOC(=O)C(=O)CC(C)=O ; O=Cc1ccc2nsnc2c1 ; Nc1ccc2[nH]cnc2c1 ; "AcOH, 120° C., 4 h" ; CC(=O)C1=C(O)C(=O)N(c2ccc3[nH]cnc3c2)C1c1ccc2nsnc2c1]

To a solution of compound 21 (600.0 mg, 3.65 mmol, 1.00 eq) in AcOH (10.0 mL) was added compound 1A (578 mg, 3.65 mmol, 516 μL, 1.00 eq) and compound 1B (486 mg, 3.65 mmol, 1.00 eq). The mixture was stirred at 120° C. for 4 hours. LCMS showed compound 21 was consumed and a main peak with the desired mass was detected. The mixture was concentrated under vacuum to give the crude product which was purified by preparative HPLC (column Phenomenex Luna C18 250×50 mm, 10 μm; mobile phase (0.05% HCl) A—water, B—ACN; gradient 5-35% B, 29 min) to give compound 21_1 (0.80 g, 1.40 mmol, 38.4% yield, 68.5% purity) as a brown solid.

LCMS: product RT=0.874 min, m/z=392.1 (M+1)⁺.

HPLC: product RT=1.706 min, 68.5% purity.

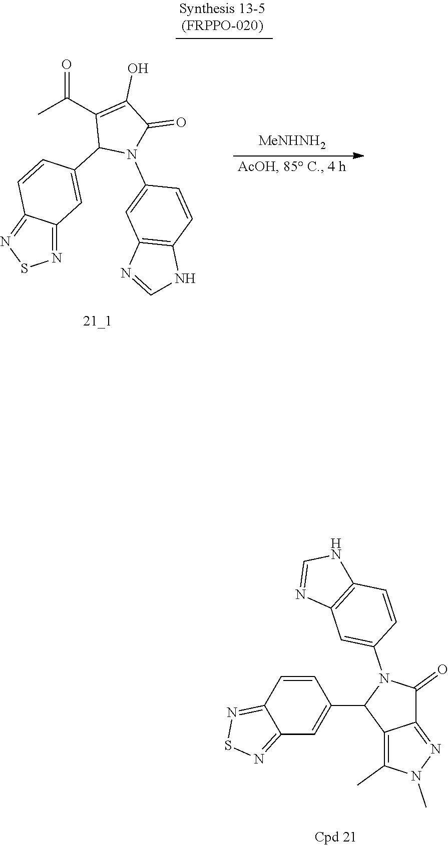 [SMILES: "MeNHNH2" ; CC(=O)C1=C(O)C(=O)N(c2ccc3[nH]cnc3c2)C1c1ccc2nsnc2c1 ; "AcOH, 85° C., 4 h" ; Cc1c2c(nn1C)C(=O)N(c1ccc3[nH]cnc3c1)C2c1ccc2nsnc2c1]

To a mixture of compound 21_1 (0.80 g, 2.04 mmol, 1.00 eq) in AcOH (15.0 mL) was added MeNHNH₂ (530 mg, 11.5 mmol, 605 μL, 5.63 eq). The mixture was stirred at 85° C. for 4 h. LCMS showed compound 21_1 was consumed and two peaks with the desired mass were detected. The mixture was concentrated under vacuum to give the crude product which was purified by preparative HPLC (column Phenomenex Synergi Max RP 150×50 mm, 10 μm; mobile phase (0.23% formic acid) A—water, B—ACN; gradient 5-35% B, 10 min) and adjusted pH to 7-8 with saturated NaHCO₃ aqueous solution. The mixture was extracted with DCM (30 mL×3). The combined organic extracts were washed with brine, dried over Na₂SO₄, filtered, and concentrated under vacuum to give Cpd 21 (6.80 mg, 16.8 μmol, 0.822% yield, 99.2% purity) as yellow solid.

LCMS: product RT=0.888 min, m/z=402.1 (M+1)⁺.

HPLC: product RT=2.291 min.

¹H NMR: 400 MHz, DMSO-d₆: δ 12.40 (br s, 1H), 8.34-8.08 (m, 2H), 7.93 (d, J=9.2 Hz, 1H), 7.77 (br s, 1H), 7.58-7.25 (m, 3H), 6.68 (br s, 1H), 3.87 (s, 3H), 2.09 (s, 3H).

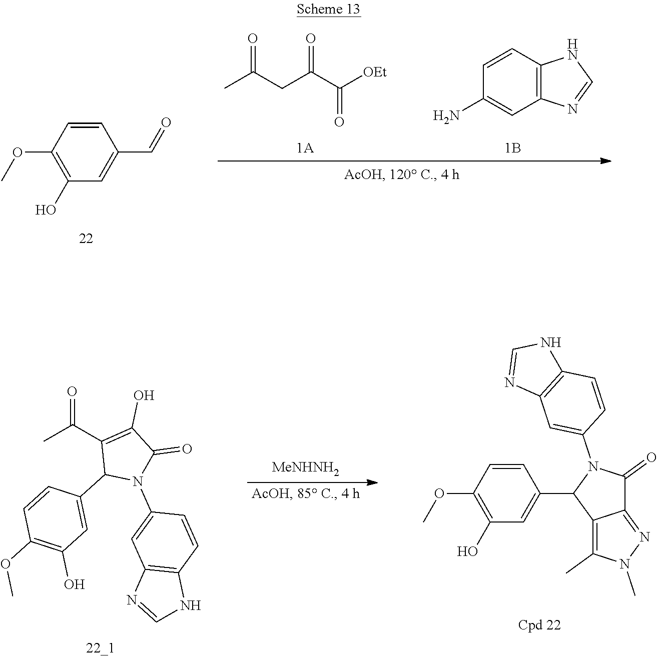

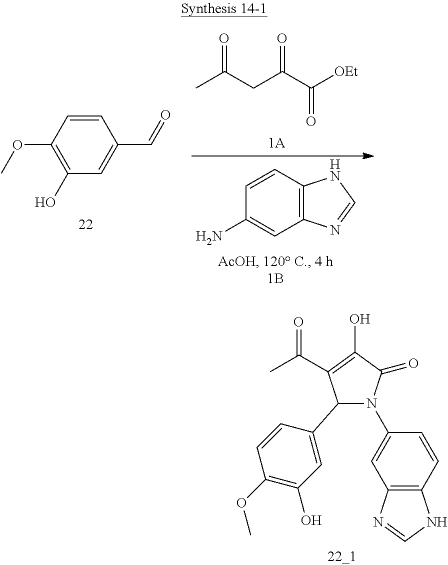

To a solution of compound 22 (5.00 g, 32.9 mmol, 1.00 eq) in AcOH (30 mL) was added compound 1B (4.38 g, 32.9 mmol, 1.00 eq) and compound 1A (5.20 g, 32.9 mmol, 4.64 mL, 1.00 eq). The mixture was stirred at 120° C. for 2 hours. LCMS showed compound 22 was consumed and a main peak with the desired mass was detected. Water (100.0 mL) was added to the mixture which was filtered and washed with CH₃CN (3×20.0 mL) to obtain a filter cake. The filter cake was dried under vacuum and the crude produce was purified by preparative HPLC (column Phenomenex Luna C18 250×50 mm, 10 μm; mobile phase (0.1% TFA) A—water, B—ACN; gradient 1-25% B, 32 min) to give compound 22_1 (1.10 g, 2.44 mmol, 7.41% yield, 84.0% purity) as a brown solid.

LCMS: product RT=0.770 min, m/z=380.1 (M+1)⁺.

HPLC: product RT=2.291 min.

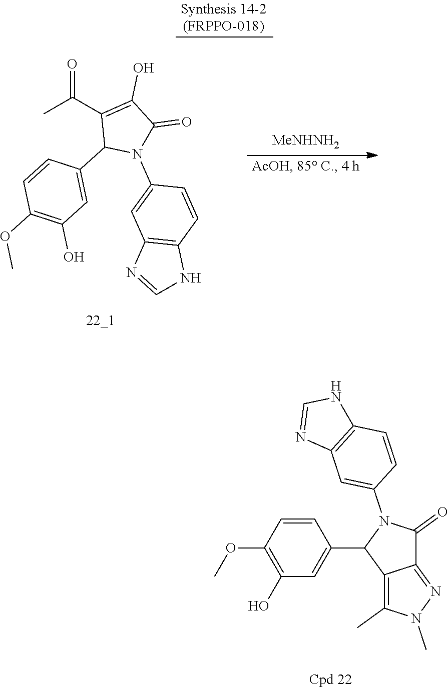 [SMILES: "MeNHNH2" ; COc1ccc(C2C(C(C)=O)=C(O)C(=O)N2c2ccc3[nH]cnc3c2)cc1O ; "AcOH, 85° C., 4 h" ; COc1ccc(C2c3c(nn(C)c3C)C(=O)N2c2ccc3[nH]cnc3c2)cc1O]

To a mixture of compound 22_1 (500 mg, 1.11 mmol, 1 eq) in AcOH (6.00 mL) was added methylhydrazine (100 mg, 2.17 mmol, 114 μL, 1.96 eq). The mixture was stirred at 85° C. for 8 hours. LCMS showed compound 22_1 was consumed and a main peak with the desired mass was detected. The crude was purified by preparative HPLC (column Waters Xbridge 150×50 mm, 10 μm; mobile phase (0.05% NH₄OH) A—water, B—ACN; gradient 8-38% B, 11.5 min) and adjusted pH to 7-8 with saturated NaHCO₃ aqueous solution. The mixture was extracted with DCM (30 mL×3). The combined organic layer was washed with brine, dried over Na₂SO₄, filtered, and concentrated under vacuum to give Cpd 22 (6.00 mg, 15.2 μmol, 1.38% yield, 98.9% purity) as off-white solid.

LCMS: product RT=0.798 min, m/z=390.1 (M+1)⁺.

HPLC: product RT=2.004 min.

¹H NMR: 400 MHz, DMSO-d₆: δ=12.41 (br s, 1H), 8.92 (s, 1H), 8.17 (d, J=4.4 Hz, 1H), 7.68 (br d, J=2.0 Hz, 1H), 7.54 (d, J=8.8 Hz, 0.5H), 7.49-7.33 (m, 1H), 7.27 (br d, J=8.8 Hz, 0.5H), 6.82-6.67 (m, 2H), 6.54 (s, 1H), 6.26 (br d, J=7.2 Hz, 1H), 3.85 (s, 3H), 3.64 (s, 3H), 2.09 (s, 3H).

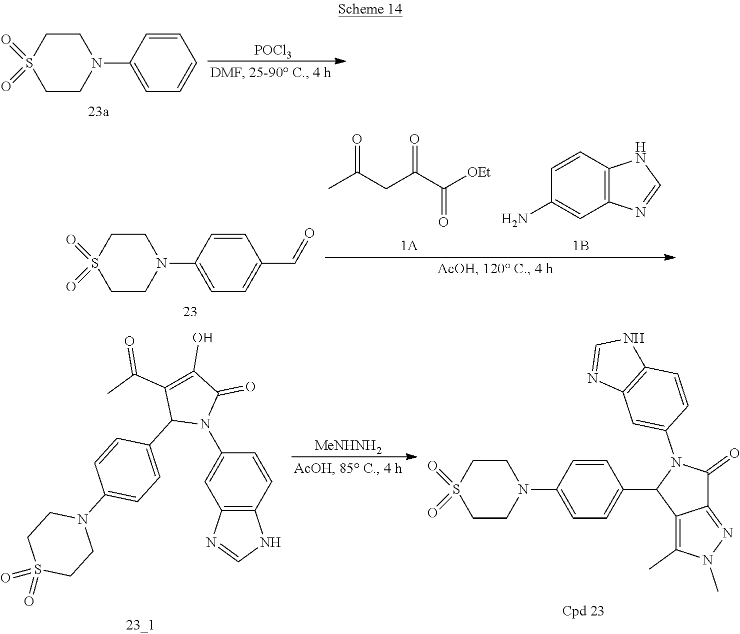

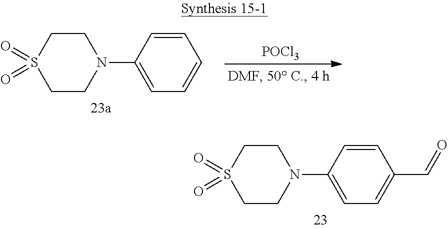 [SMILES: O=S1(=O)CCN(c2ccccc2)CC1 ; "POCl3" ; O=Cc1ccc(N2CCS(=O)(=O)CC2)cc1]

To a solution of compound 23a (3.00 g, 14.2 mmol, 1.00 eq) in DMF (12 mL) was added POCl₃ (3.27 g, 21.3 mmol, 1.98 mL, 1.50 eq) dropwise at 25° C. and the temperature maintained below 35° C. The reaction vessel was heated to 80-90° C. and stirred for 4 hours. LCMS showed that compound 23a was consumed and the desired mass was detected. The reaction mixture was cooled to 20° C. and then poured into ice-water (60 mL). The mixture was adjusted pH to 8 with solid NaHCO₃. The mixture was stirred for 0.5 hour before filtering. The filter cake was dried under vacuum to give compound 23 (2.4 g, 8.83 mmol, 62.1% yield, 88.0% purity) as a light yellow solid, which was used directly without purification.

LCMS: product RT=0.457 min, m/z=167.1 (M+1)⁺.

¹H NMR: 400 MHz, DMSO-d₆: δ=9.79 (d, J=8.8 Hz, 1H), 7.78 (d, J=9.2 Hz, 2H), 7.18 (d, J=8.8 Hz, 2H), 3.98 (t, J=0.8 Hz, 4H), 3.15 (t, J=4.8 Hz, 4H).

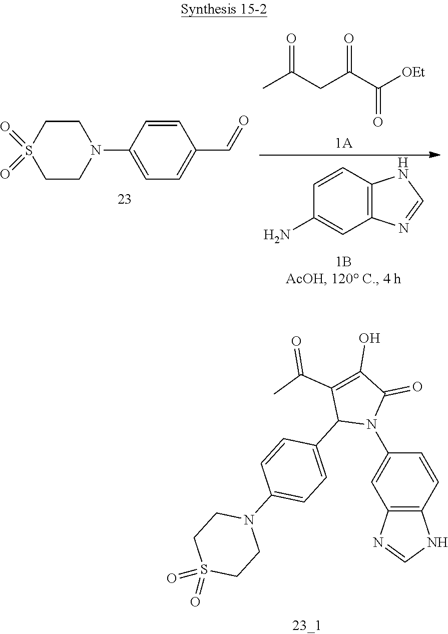

To a solution of compound 23 (0.60 g, 2.51 mmol, 1.00 eq) in AcOH (10.0 mL) was added compound 1A (594 mg, 3.76 mmol, 531 μL, 1.50 eq) and compound 1B (500 mg, 3.76 mmol, 1.50 eq). The mixture was stirred at 120° C. for 12 hours. LCMS showed compound 23 was consumed and a main peak with the desired mass was detected. The mixture was concentrated under vacuum to give the crude product which was purified by reversed-phase C18 column chromatography (0.1% formic acid, 0-25% MeCN in water) to give compound 23_1 (0.10 g, 90.7 μmol, 3.62% yield, 42.3% purity) as a brown solid.

LCMS: product RT=0.858 min, m/z=467.0 (M+1)⁺.

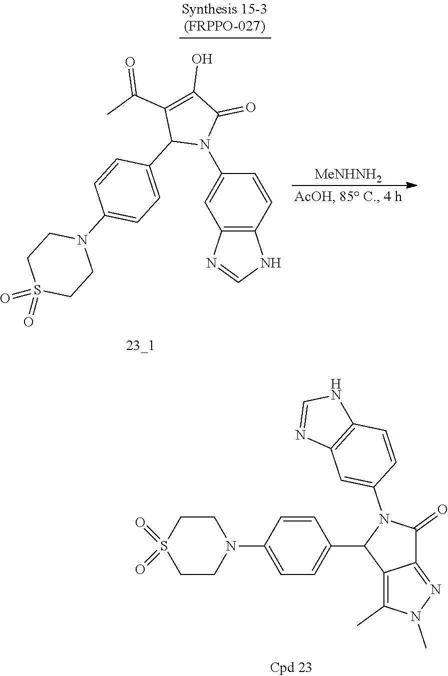 [SMILES: "MeNHNH2" ; CC(=O)C1=C(O)C(=O)N(c2ccc3[nH]cnc3c2)C1c1ccc(N2CCS(=O)(=O)CC2)cc1 ; Cc1c2c(nn1C)C(=O)N(c1ccc3[nH]cnc3c1)C2c1ccc(N2CCS(=O)(=O)CC2)cc1]

To a mixture of compound 23_1 (100 mg, 214 μmol, 1.00 eq) in AcOH (15.0 mL) was added MeNHNH₂ (224 mg, 4.87 mmol, 256 μL, 22.7 eq). The mixture was stirred at 85° C. for 8 hours. LCMS showed compound 23_1 was consumed and two main peaks with the desired mass were detected. The mixture was concentrated under vacuum to give the crude product. The crude product was purified by preparative HPLC (column Phenomenex Synergi Max RP 150×50 mm, 10 μm; mobile phase (0.23% formic acid) A—water, B—ACN; gradient 1-31% B, 10 min) to give Cpd 23 (2.40 mg, 4.01 μmol, 1.87% yield, 79.6% purity) as a brown solid.

LCMS: product RT=0.951 min, m/z=477.2 (M+1)⁺.

HPLC: product RT=2.085 min

¹H NMR: 400 MHz, DMSO-d₆: δ=12.39 (br s, 1H), 8.16 (s, 1H), 7.69 (s, 1H), 7.48 (s, 1H), 7.33 (s, 1H), 7.09 (d, J=4.4 Hz, 2H), 6.86 (d, J=8.4 Hz, 2H), 6.33 (s, 1H), 3.85 (s, 3H), 3.64 (s, 4H), 3.04 (s, 4H), 2.09 (s, 3H).

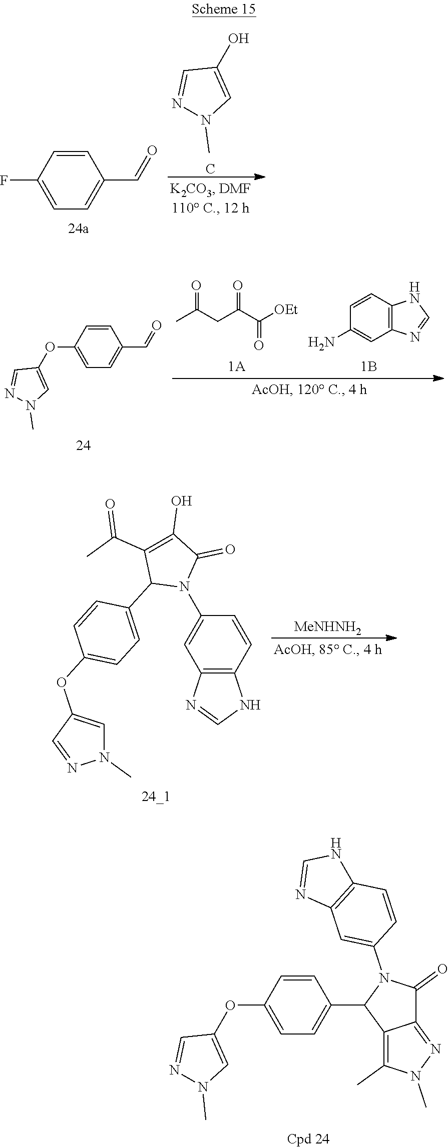

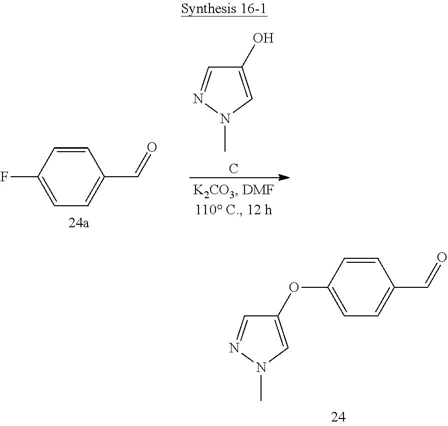

A mixture of compound 24a (2.53 g, 20.3 mmol, 2.14 mL, 1.00 eq), compound C (2.00 g, 20.3 mmol, 1.00 eq), K₂CO₃ (4.23 g, 30.5 mmol, 1.50 eq) in DMF (20.0 mL) was heated to 110° C. under N₂ and stirred for 12 hours. LCMS showed 24a was consumed completely and the desired mass was detected. Water (100 mL) was added to the mixture and the mixture extracted with ethyl acetate (100 mL×2), the combined organic extracts were dried by Na₂SO₄, filtered and concentrated on vacuum. The crude product was purified by column chromatography (SiO₂, petroleum ether/ethyl acetate=1:0 to 20:1, R_{f}=0.7) to give compound 24 (0.95 g, 4.67 mmol, 22.9% yield, 99.4% purity) as a yellow oil.

LCMS: product RT=0.781 min, m/z=203.0 (M+1)⁺.

HPLC: product RT=1.692 min, 99.4% purity.

¹H NMR: 400 MHz, DMSO-d₆: δ 9.91 (s, 1H), 7.83 (d, J=8.8 Hz, 2H), 7.35 (d, J=19.6 Hz, 2H), 7.11 (d, J=8.4 Hz, 2H), 4.0 (s, 3H).

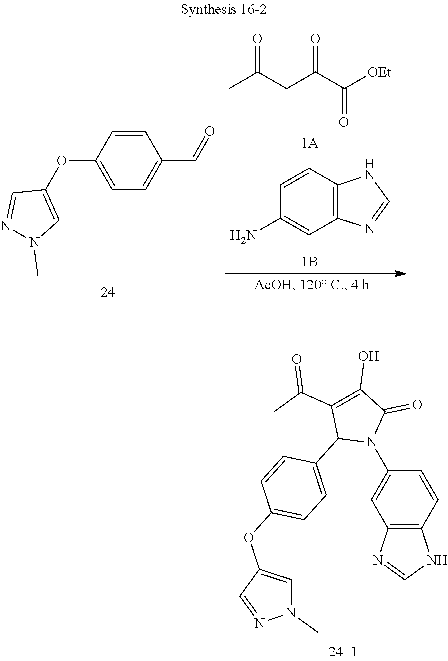

To a solution of compound 24 (600 mg, 3.65 mmol, 1.00 eq) in AcOH (10.0 mL) was added compound 1A (753 mg, 4.76 mmol, 672 μL, 1.00 eq) and compound 1B (634 mg, 4.76 mmol, 1.00 eq). The mixture was stirred at 120° C. for 12 hours. LCMS showed reactant 24 was consumed and a peak with the desired mass was detected. The mixture was concentrated under vacuum to give crude product. The crude product was purified by reversed-phase C18 column chromatography (0.1% formic acid, 0-25% MeCN in water) to give compound 24_1 (0.75 g, 787 μmol, 16.5% yield, 45.1% purity) as black brown solid.

LCMS: product RT=0.881 min, m/z=430.1 (M+1)⁺.

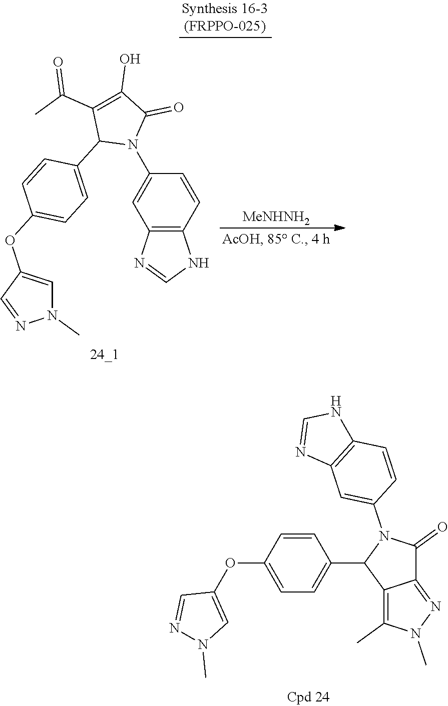 [SMILES: "MeNHNH2" ; CC(=O)C1=C(O)C(=O)N(c2ccc3[nH]cnc3c2)C1c1ccc(Oc2cnn(C)c2)cc1 ; Cc1c2c(nn1C)C(=O)N(c1ccc3[nH]cnc3c1)C2c1ccc(Oc2cnn(C)c2)cc1]

To a mixture of compound 24_1 (0.70 g, 1.63 mmol, 1.00 eq) in AcOH (10.0 mL) was added MeNHNH₂ (150 mg, 3.26 mmol, 171 μL, 2.00 eq). The mixture was stirred at 85° C. for 8 hours. LCMS showed compound 24_1 was consumed and a peak with the desired mass was detected. The mixture was concentrated under vacuum to give the crude product. The crude was purified by preparative HPLC (column Phenomenex Synergi Max RP 150×50 mm, 10 μm; mobile phase (0.2% formic acid) A—water, B—ACN; gradient 1-31% B, 10 min) to give Cpd 24 (6.78 mg, 15.3 μmol, 0.941% yield, 99.4% purity) as a yellow solid.

LCMS: product RT=0.908 min, m/z=440.2 (M+1)⁺.

HPLC: product RT=2.302 min.

¹H NMR: 400 MHz, DMSO-d₆: δ=12.41 (br s, 1H), 8.18 (d, J=3.6 Hz, 1H), 7.76-7.61 (m, 2H), 7.57-7.42 (m, 1H), 7.41-7.25 (m, 2H), 7.20 (d, J=8.7 Hz, 2H), 6.83 (d, J=8.0 Hz, 2H), 6.39 (d, J=8.8 Hz, 1H), 3.86 (s, 3H), 3.76 (s, 3H), 2.10 (s, 3H).

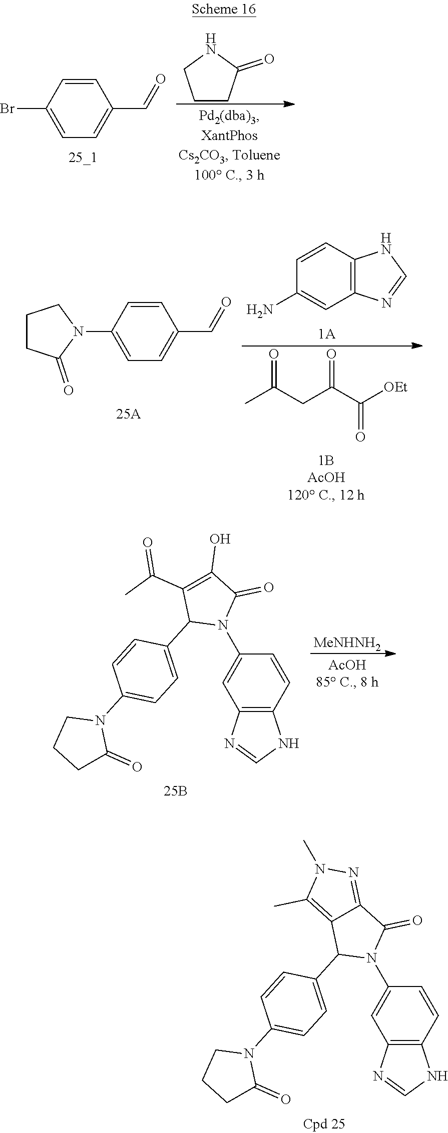

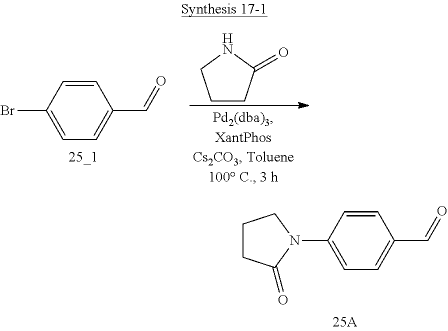

To a solution of compound 25_1 (2.00 g, 10.8 mmol, 1.00 eq), pyrrolidin-2-one (1.10 g, 13.0 mmol, 994 μL, 1.20 eq) and Cs₂CO₃ (10.6 g, 32.4 mmol, 3.00 eq) in toluene (20 mL) was added Pd₂(dba)₃ (495 mg, 540 μmol, 0.05 eq) and XantPhos (625 mg, 1.08 mmol, 0.10 eq) under N₂. The mixture was stirred at 100° C. for 3 hours under N₂ atmosphere. LCMS showed compound 25_1 was consumed completely and the desired mass was detected. The reaction mixture was filtered and the cake was washed with ethyl acetate (30 mL×5). The organic filtrate was concentrated under reduced pressure to give a residue which was purified by column chromatography (SiO₂, petroleum ether:ethyl acetate=1:0 to 0:1), TLC (petroleum ether:ethyl acetate=0:1, R_{f} of compound 25_1 was 0.7, R_{f} of compound 25A was 0.5). Compound 25A (0.85 g, 4.49 mmol, 41.6% yield, 100% purity) was obtained as an off-white solid.

LCMS: product: RT=0.702 min, m/z=190.0 (M+H)⁺.

¹H NMR: 400 MHz, DMSO-d₆: δ 9.92 (s, 1H), 7.91 (s, 4H), 3.90 (t, J=7.0 Hz, 2H), 2.56 (t, J=8.0 Hz, 2H), 2.11-2.07 (m, 2H).

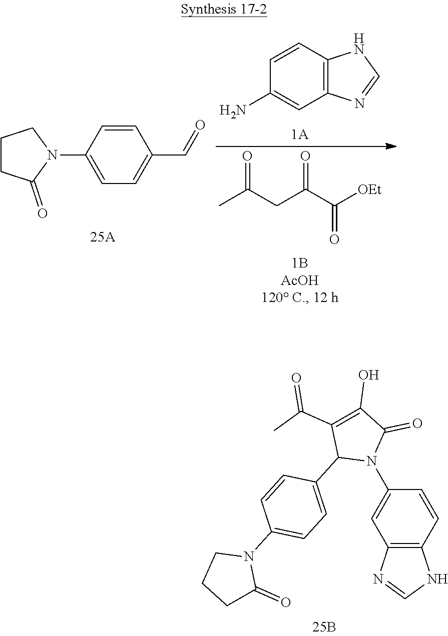

To a solution of compound 25A (600 mg, 3.17 mmol, 1.00 eq) in AcOH (6.00 mL) was added compound 1A (422 mg, 3.17 mmol, 1.00 eq) and compound 1B (501 mg, 3.17 mmol, 447 μL, 1.00 eq) and the mixture was stirred at 120° C. for 12 hours. LCMS and HPLC showed compound 25A was consumed completely. The reaction mixture was concentrated under reduced pressure to give a residue which was purified by HPLC (column Phenomenex Luna C18 250×50 mm, 10 μm; mobile phase (0.23% formic acid) A—water, B—ACN; gradient 5-35% B, 32 min) to give the product compound 25B (220.0 mg, 373.5 μmol, 11.8% yield, 70.7% purity) as a yellow solid.

LCMS: product: RT=0.688 min; m/z=417.0 (M+H)⁺.

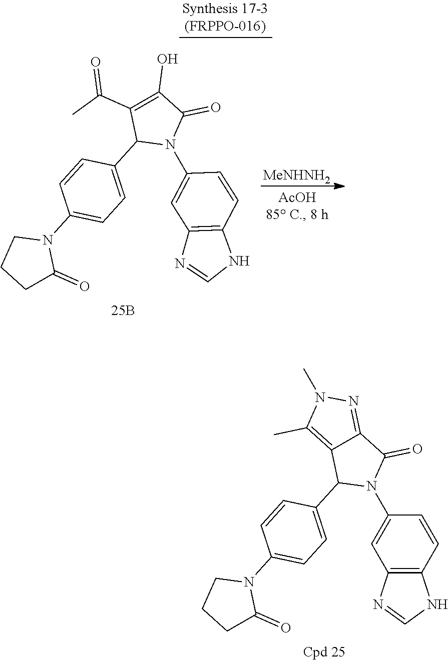 [SMILES: "MeNHNH2" ; CC(=O)C1=C(O)C(=O)N(c2ccc3[nH]cnc3c2)C1c1ccc(N2CCCC2=O)cc1 ; Cc1c2c(nn1C)C(=O)N(c1ccc3[nH]cnc3c1)C2c1ccc(N2CCCC2=O)cc1]

To a solution of compound 25B (200 mg, 480 μmol, 1.00 eq) in AcOH (2.00 mL) was added methylhydrazine (44.3 mg, 960.6 μmol, 50.6 μL, 2.00 eq). The mixture was stirred at 85° C. for 8 hours. LCMS and HPLC showed compound 25B was consumed completely. The mixture was concentrated under reduced pressure and the residue was purified by HPLC (column Phenomenex Luna C18 200×40 mm, 10 μm; mobile phase (0.1% TFA) A—water, B—ACN; gradient 10-30% B, 10 min) to give the product, compound Cpd 25 (14.8 mg, 34.1 μmol, 7.1% yield, 98.5% purity) as a yellow solid. The residue was repurified by HPLC (column Phenomenex Gemini 150×25 mm, 10 μm; mobile phase (10 mM NH₄HCO₃) A—water, B—ACN; gradient 13-43% B, 10 min) to give the product Cpd 25 (14.7 mg, 34.0 μmol, 7.0% yield, 98.1% purity) as a yellow solid.

LCMS: product: RT=0.795 min; m/z=427.3 (M+H)⁺.

LCMS: product: RT=0.761 min; m/z=853.6 (2M+H)⁺.

HPLC: product: RT=1.828 min.

¹H NMR: 400 MHz, DMSO-d₆: δ 12.41 (s, 1H), 8.17 (s, 1H), 7.70 (s, 1H), 7.53-7.51 (d, J=8.80 Hz, 2H), 7.25 (s, 2H), 7.23 (s, 2H), 6.40 (s, 1H), 3.86 (s, 3H), 3.73-3.69 (m, 2H), 2.41 (t, J=8.00 Hz, 2H), 2.09 (s, 3H), 1.99-1.95 (m, 2H).

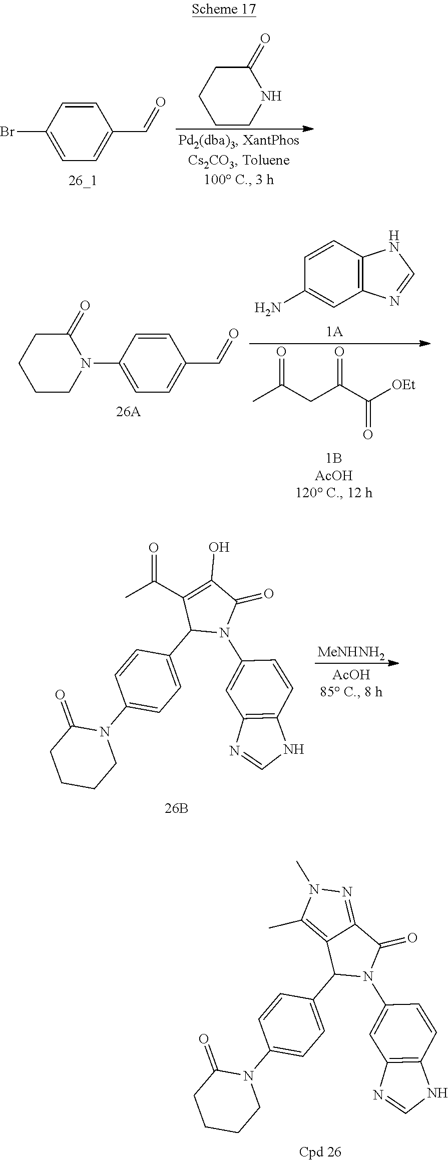

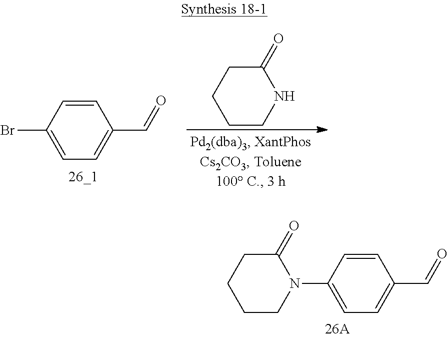 [SMILES: O=Cc1ccc(Br)cc1 ; O=C1CCCCN1 ; O=Cc1ccc(N2CCCCC2=O)cc1]

A mixture of compound 26_1 (2.00 g, 10.8 mmol, 1.00 eq), piperidin-2-one (1.29 g, 13.0 mmol, 1.20 eq), XantPhos (625 mg, 1.08 mmol, 0.10 eq), Cs₂CO₃ (10.6 g, 32.4 mmol, 3.00 eq) and Pd₂(dba)₃ (494 mg, 540 μmol, 0.05 eq) in toluene (20 mL) was degassed and purged with N₂ for 3 times, and then the mixture was stirred at 100° C. for 12 hours under N₂ atmosphere. LCMS showed compound 26_1 was consumed completely and the desired mass was detected. The reaction mixture was filtered and the crude product was purified by flash silica gel chromatography (SiO₂, petroleum ether/ethyl acetate=50:1 to 1:1, TLC petroleum ether:ethyl acetate=0:1, the product compound 26A R_{f}=0.3) to give the product, compound 26A (0.96 g, 4.54 mmol, 42.0% yield, 96.5% purity) as a brown oil.

LCMS: product: RT=0.570 min, m/z=204.1 (M+H)⁺.

HPLC: product: RT=1.377 min, 96.5% purity.

¹H NMR: 400 MHz, CDCl₃: δ 9.98 (s, 1H), 7.91-7.88 (m, 2H), 7.49-7.46 (m, 2H), 3.73-3.70 (m, 2H), 2.59 (t, J=6.4 Hz, 2H), 2.00-1.70 (m, 4H).

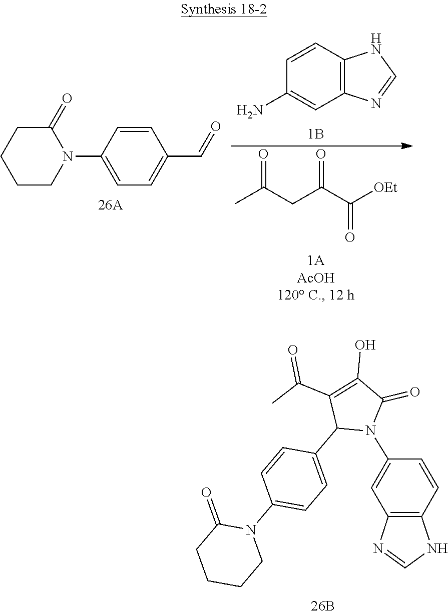

To a solution of compound 26A (900.0 mg, 4.43 mmol, 1.00 eq) in AcOH (13.0 mL) was added compound 1B (589 mg, 4.43 mmol, 1.00 eq) and compound 1A (700 mg, 4.43 mmol, 625 μL, 1.00 eq) and the mixture was stirred at 120° C. for 12 hours. LCMS (desired product: RT=0.691 min; m/z=431.2 (M+H)⁺) and HPLC showed compound 26A was consumed completely. The reaction mixture was concentrated under reduced pressure to give a residue which was purified by HPLC (column Phenomenex Luna C18 250×50 mm, 10 μm; mobile phase (0.23% formic acid) A—water, B—ACN; gradient 5-35% B, 32 min) to give the product compound 26B (400.0 mg, 679.3 μmol, 15.3% yield) as a yellow solid.

LCMS (crude): product: RT=0.691 min; m/z=431.2 (M+H)⁺ HPLC (crude): 73.1% purity.

LCMS: product: RT=0.692 min; m/z=431.0 (M+H)⁺.

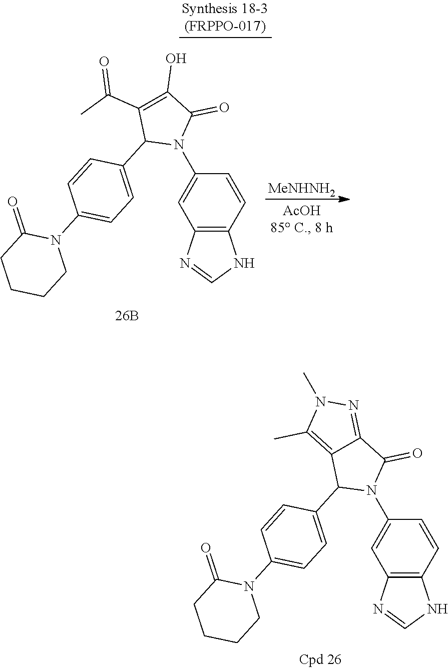 [SMILES: "MeNHNH2" ; CC(=O)C1=C(O)C(=O)N(c2ccc3[nH]cnc3c2)C1c1ccc(N2CCCCC2=O)cc1 ; Cc1c2c(nn1C)C(=O)N(c1ccc3[nH]cnc3c1)C2c1ccc(N2CCCCC2=O)cc1]

To a solution of compound 26B (400 mg, 929 μmol, 1.00 eq) in AcOH (6.00 mL) was added methylhydrazine (85.6 mg, 1.86 mmol, 97.9 μL, 2.00 eq). The mixture was stirred at 85° C. for 8 hours. LCMS and HPLC showed compound 26B was consumed completely. The mixture was concentrated under reduced pressure and the residue was purified by HPLC (column Phenomenex Luna C18 200×40 mm, 10 μm; mobile phase (0.1% TFA) A—water, B—ACN; gradient 10-30% B, 10 min) to give the product, Cpd 26 (91.0 mg, 194.2 μmol, 20.9% yield, 94.0% purity) as a yellow solid. The residue was repurified by HPLC (column Phenomenex Gemini 150×25 mm, 10 μm; mobile phase (10 mM NH₄HCO₃) A—water, B—ACN; gradient 11-41% B, 10 min) to give the product, Cpd 26 (91.0 mg, 203 μmol, 98.4% yield, 98.4% purity) as a yellow solid.

LCMS: product: RT=0.757 min; m/z=441.3 (M+H)⁺, 881.6 (2M+H)⁺.

HPLC: product: RT=1.814 min.

¹H NMR: 400 MHz, DMSO-d₆: δ 12.43 (s, 1H), 8.17 (s, 1H), 7.76 (s, 1H), 7.50-7.38 (m, 2H), 7.27-7.25 (d, J=8.40 Hz, 2H), 7.18-7.15 (d, J=8.80 Hz, 2H), 6.47 (s, 1H), 3.86 (s, 3H), 3.52-3.45 (m, 2H), 2.32 (t, J=5.60 Hz, 2H), 2.12 (s, 3H), 1.77-1.75 (m, 4H).

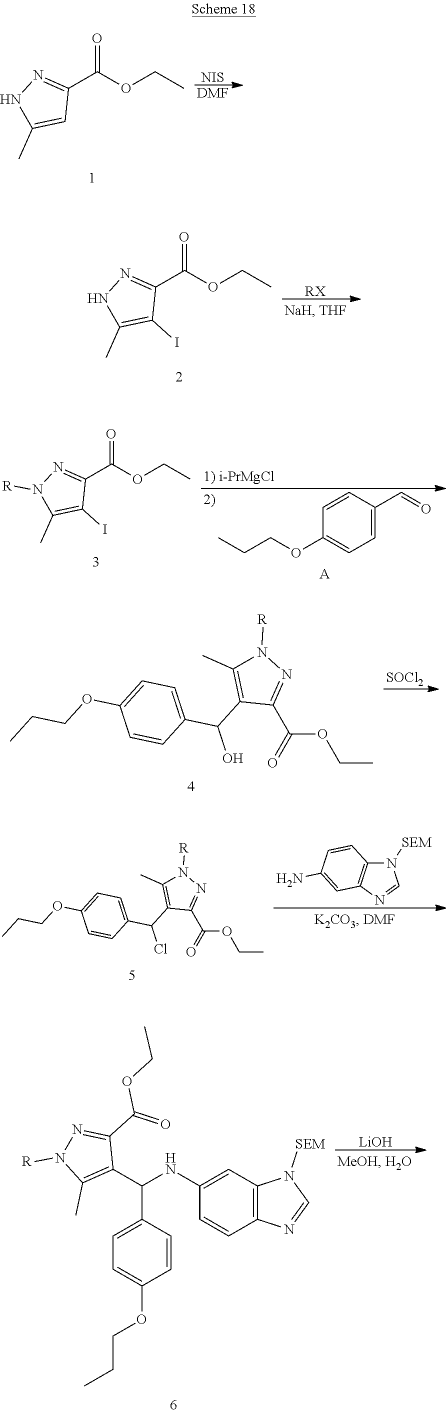

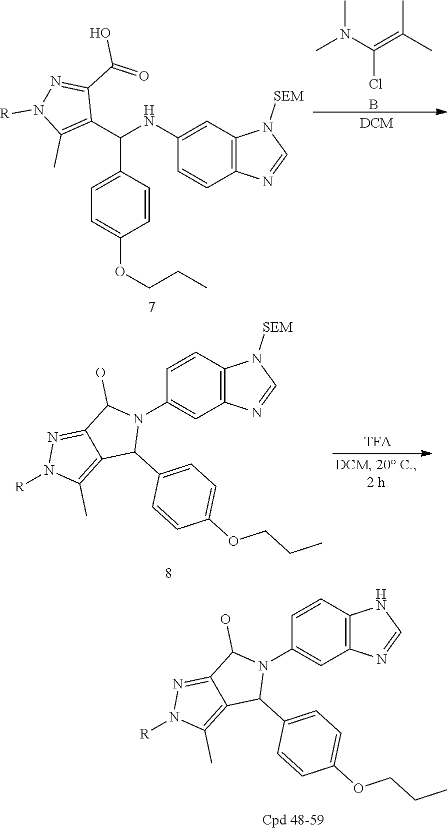

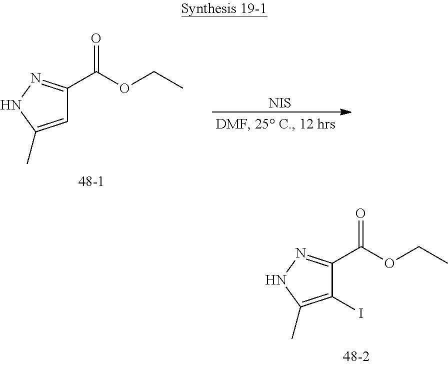

To a solution of compound 48-1 (5.00 g, 32.4 mmol, 1.00 eq) in DMF (40 mL) was added NIS (8.76 g, 38.9 mmol, 1.20 eq). The mixture was stirred at 25° C. for 12 hours. LCMS showed compound 48-1 was consumed completely and the desired mass was detected. The reaction mixture was concentrated under reduced pressure to give a residue which was diluted with water 60 mL and extracted with EtOAc (50 mL×3). The combined organic extracts were washed with brine (30 mL×2), dried over Na₂SO₄, filtered and concentrated under reduced pressure to give compound 48-2 (10.0 g, crude) as a yellow solid.

LCMS: RT=0.845 min, m/z=280.9 (M+1)⁺.

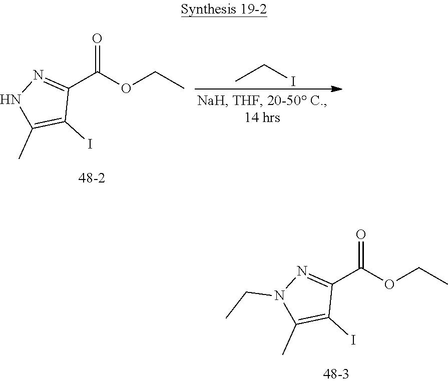

To a solution of compound 48-2 (4.00 g, 14.3 mmol, 1.00 eq) in THF (20 mL) was added NaH (1.71 g, 42.9 mmol, 60.0% purity, 3.00 eq). The mixture was stirred at 20° C. for 0.5 hours. Iodoethane (2.67 g, 17.1 mmol, 1.37 mL, 1.20 eq) was added and the mixture was stirred at 20° C. for 1.5 hours. Then the mixture was stirred at 50° C. for 12 hours. The reaction mixture was quenched by addition water (60 mL) and extracted with EtOAc (50 mL×3). The combined organic layers were washed with brine (30 mL×2), dried over Na₂SO₄, filtered and concentrated under reduced pressure to give compound 48-3 (3.80 g, 12.3 mmol, 86.4% yield) as a yellow oil.

¹H NMR: 400 MHz DMSO: δ 4.29-4.20 (m, 4H), 2.33 (s, 3H), 1.32-1.28 m, 6H).

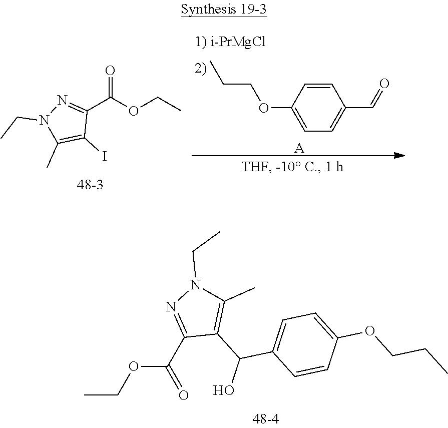

To a mixture of compound 48-3 (3.00 g, 9.74 mmol, 1.00 eq) in THF (100 mL) was added iPrMgCl (1.3 M, 8.24 mL, 1.10 eq) drop-wise at 0° C. under N₂. The mixture was stirred at 0° C. for 20 min, then the mixture was cooled to −20° C., a solution of compound A (1.60 g, 9.74 mmol, 1.54 mL, 1.00 eq) dissolved in THF (10 mL) was added to the mixture and the mixture was stirred at −20° C. under N₂ for 40 min. TLC (PE/EtOAc=1/1, starting material R_{f}=0.8, product R_{f}=0.5) showed a new compound was present in the reaction mixture. The reaction mixture was poured into saturated NH₄Cl (60 mL) and extracted with ethyl acetate (100 mL×3), the organic layer was washed with brine (50 mL×3), dried over Na₂SO₄, filtered and concentrated under reduced pressure. The residue was purified by column chromatography (SiO₂, petroleum ether/ethyl acetate=20:1 to 2:1; TLC, petroleum ether/ethyl acetate=1:1, product R_{f}=0.5) to give compound 48-4 (2.10 g, 6.06 mmol, 62.3% yield) as a light yellow oil.

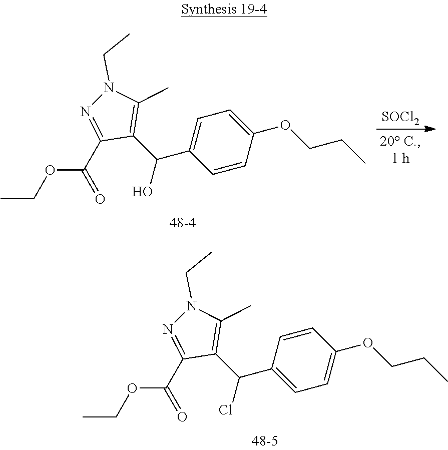

A mixture of compound 48-4 (500 mg, 1.44 mmol, 1.00 eq) and SOCl₂ (6.56 g, 55.1 mmol, 4 mL, 38.2 eq) was stirred at 20° C. for 1 hour. The reaction mixture was concentrated under reduced pressure to give compound 48-5 (526 mg, 1.44 mmol, 99.9% yield) as a yellow oil.

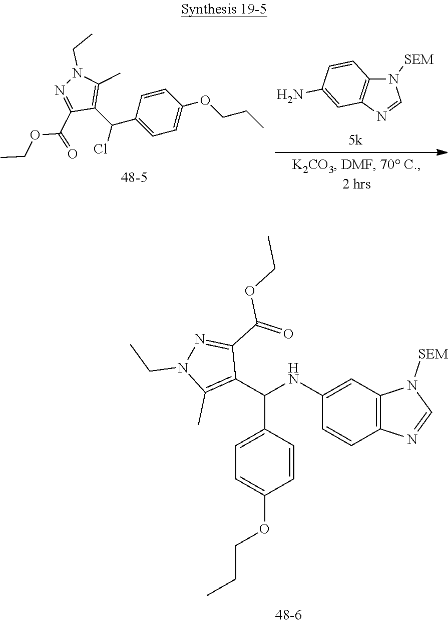

To a solution of compound 48-5 (526 mg, 1.44 mmol, 1.00 eq) in DMF (5 mL) was added K₂CO₃ (430 mg, 3.11 mmol, 2.16 eq) and compound 5k (430 mg, 1.63 mmol, 1.13 eq). The mixture was stirred at 70° C. for 2 hours. The desired product mass was detected by LCMS. The reaction mixture was filtered and concentrated under reduced pressure to give a residue which was purified by reversed-phase HPLC (0.1% formic acid H₂O/MeCN) to give Compound 48-6 (800 mg, 1.35 mmol, 93.8% yield) as a yellow oil.

LCMS: RT=0.957 min, m/z=592.4 (M+1)⁺.

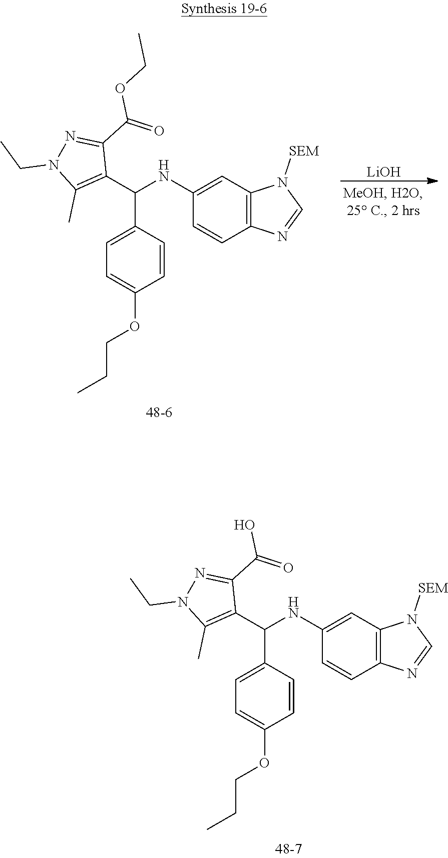 [SMILES: CCCOc1ccc(C(Nc2ccc3ncn(COCC[Si](C)(C)C)c3c2)c2c(C(=O)OCC)nn(CC)c2C)cc1 ; "LiOH" ; CCCOc1ccc(C(Nc2ccc3ncn(COCC[Si](C)(C)C)c3c2)c2c(C(=O)O)nn(CC)c2C)cc1]

To a solution of compound 48-6 (400 mg, 676 μmol, 1.00 eq) in MeOH (6 mL) and H₂O (3 mL) was added LiOH.H₂O (500 mg, 11.9 mmol, 17.6 eq). The mixture was stirred at 20° C. for 2 hours. LCMS showed compound 48-6 was consumed completely and the desired mass was detected. The reaction mixture was concentrated under reduced pressure to remove MeOH. The residue was diluted with water (20 mL), pH adjusted to 5, and then extracted with EtOAc (30 mL×3). The combined organic extracts were washed with brine (30 mL×1), dried over Na₂SO₄, filtered and concentrated under reduced pressure to give compound 48-7 (250 mg, 443 μmol, 65.6% yield) as a yellow oil which was used in the next reaction without further purification.

LCMS: RT=0.933 min, m/z=564.3 (M+1)⁺.

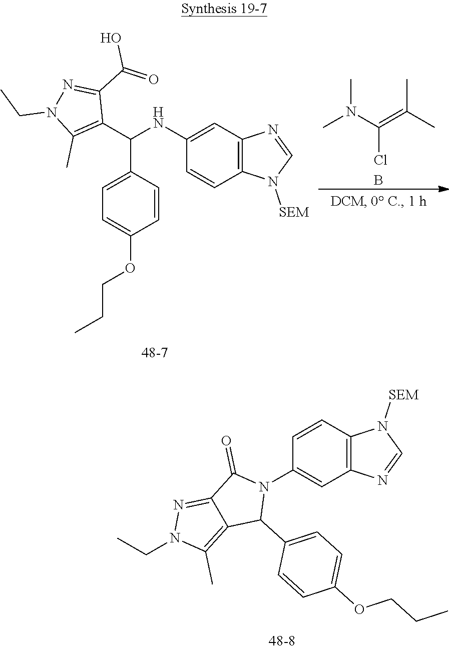

A mixture of compound 48-7 (250 mg, 443 μmol, 1.00 eq) and compound B (90 mg, 674 μmol, 89 μL, 1.52 eq) in DCM (6 mL) was stirred at 0° C. for 1 hour. The desired product mass was detected by LCMS. The reaction mixture was concentrated under reduced pressure to give a residue which was purified by reversed-phase HPLC (0.1% formic acid H₂O/MeCN) to give compound 48-8 (100 mg, 183 μmol, 41% yield) as a yellow oil.

LCMS: RT=0.948 min, m/z=546.3 (M+1)⁺.

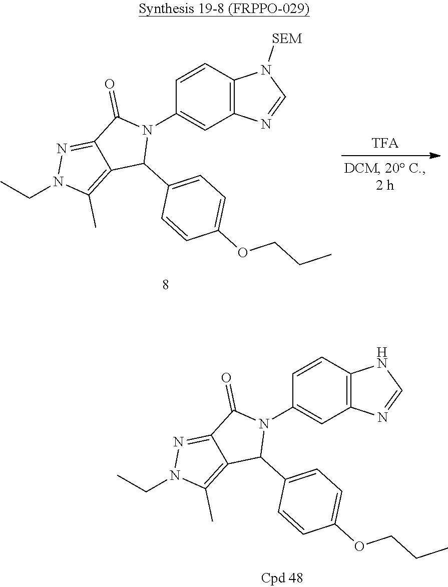

A solution of compound 48-8 (40 mg, 73.3 μmol, 1.00 eq) and TFA (6.16 g, 54.0 mmol, 4 mL, 737eq) in DCM (4 mL) was stirred at 20° C. for 2 hours. The reaction mixture was concentrated under reduced pressure to give a residue which was purified by reversed-phase HPLC (0.1% NH₄OH, H₂O/MeCN) to give Cpd 48 (16 mg, 38.3 μmol, 52.2% yield, 99.4% purity) as a yellow solid.

LCMS: RT=0.910 min, m/z=416.4 (M+1)⁺.

HPLC: RT=1.673 min.

¹H NMR: 400 MHz DMSO: δ 8.18 (s, 1H), 7.67 (s, 1H), 7.49-7.47 (d, J=4.8 Hz, 1H), 7.32-7.29 (m, 1H), 7.14-7.08 (m, 2H), 6.78-6.76 (d, J=8.8 Hz, 2H), 6.43 (s, 1H), 4.19-4.14 (m, 2H), 3.81-3.76 (m, 2H), 2.07 (s, 3H), 1.68-1.59 (m, 2H), 1.44-1.35 (m, 3H), 0.92-0.88 (m, 3H).

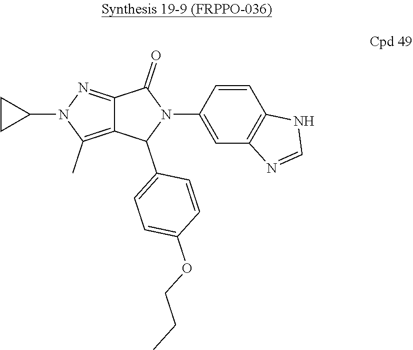 [SMILES: CCCOc1ccc(C2c3c(nn(C4CC4)c3C)C(=O)N2c2ccc3[nH]cnc3c2)cc1]

The compound was obtained using an analogous method. Cpd 49 (21.8 mg, 50.3 μmol, 35.1% yield, 98.5% purity) was obtained as a white solid.

LCMS: RT=0.827 min, m/z=428.2 (M+1)⁺.

HPLC: RT=1.706 min.

¹H NMR: 400 MHz DMSO: δ 12.4 (s, 1H), 8.16-8.15 (m, 1H), 7.66-7.54 (m, 1H), 7.52-7.41 (m, 2H), 7.35-7.12 (m, 2H), 6.78-6.76 (m, 2H), 6.33 (s, 1H), 3.83-3.77 (m, 2H), 3.68-3.64 (m, 1H), 2.17 (s, 3H), 1.66-1.61 (m, 2H), 1.10-1.05 (m, 4H), 0.92-0.88 (m, 3H).

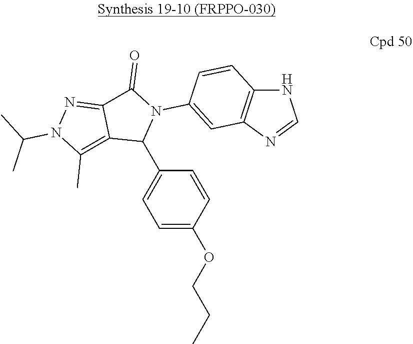 [SMILES: CCCOc1ccc(C2c3c(nn(C(C)C)c3C)C(=O)N2c2ccc3[nH]cnc3c2)cc1]

The compound was obtained using an analogous method. Cpd 50 (6.95 mg, 16.0 μmol, 18.0% yield, 99.0% purity) was obtained as light yellow solid.

LCMS: RT=0.949 min, m/z=430.5 (M+1)⁺.

HPLC: RT=2.036 min.

¹H NMR: 400 MHz DMSO: δ 12.39-12.37 (m, 1H), 8.16 (s, 1H), 7.66 (s, 1H), 7.54-7.26 (m, 2H), 7.24-7.12 (m, 2H), 6.78-6.76 (d, J=8.2 Hz, 2H), 6.34-6.32 (d, J=7.8 Hz, 1H), 4.61-4.53 (m, 1H), 3.83-3.75 (m, 2H), 2.11 (s, 3H), 1.66-1.59 (m, 2H), 1.45-1.41 (m, 6H), 0.92-0.88 (m, 3H).

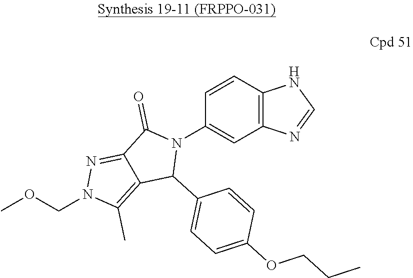 [SMILES: CCCOc1ccc(C2c3c(nn(COC)c3C)C(=O)N2c2ccc3[nH]cnc3c2)cc1]

The compound was obtained using an analogous method. Cpd 51 (18 mg, 41.13 μmol, 46.21% yield, 98.6% purity) was obtained as an off-white solid

LCMS: RT=0.903 min, m/z=432.4 (M+1)⁺.

HPLC: RT=1.607 min.

¹H NMR: 400 MHz DMSO: δ 8.17 (s, 1H), 7.67 (s, 1H), 7.52-7.44 (d, J=32 Hz, 1H), 7.34-7.28 (d, J=23.2 Hz, 1H), 7.15-7.13 (d, J=8.4 Hz, 2H), 6.78-6.76 (d, J=8.4 Hz, 2H), 6.39 (s, 1H), 5.54-5.45 (m, 2H), 3.81-3.75 (m, 2H), 3.23 (s, 3H), 2.11 (s, 3H), 1.68-1.59 (m, 2H), 0.92-0.88 (m, 3H).

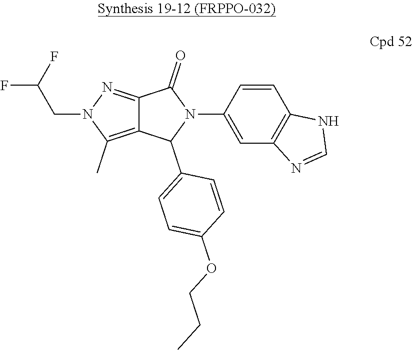 [SMILES: CCCOc1ccc(C2c3c(nn(CC(F)F)c3C)C(=O)N2c2ccc3[nH]cnc3c2)cc1]

The compound was obtained using an analogous method. Cpd 52 (7.15 mg, 15.6 μmol, 30.2% yield, 98.2% purity) was obtained as an off-white solid.

LCMS: RT=0.925 min, m/z=452.3 (M+1)⁺.

HPLC: RT=1.691 min.

¹H NMR: 400 MHz MeOH: δ 8.53-8.23 (m, 1H), 7.66-7.57 (m, 2H), 7.37-7.34 (m, 1H), 7.15-7.08 (m, 2H), 6.82-6.77 (m, 2H), 6.42-6.14 (m, 2H), 4.71-4.63 (m, 2H), 3.88-3.81 (m, 2H), 2.38 (s, 1H), 2.18 (s, 1H), 2.05 (s, 1H), 1.74-1.69 (m, 2H), 1.01-0.96 (m, 3H).

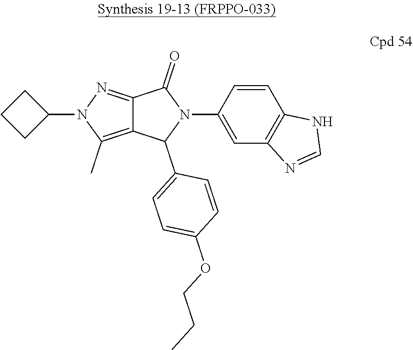 [SMILES: CCCOc1ccc(C2c3c(nn(C4CCC4)c3C)C(=O)N2c2ccc3[nH]cnc3c2)cc1]

The compound was obtained using an analogous method. Cpd 54 (25.8 mg, 33.3% yield, 99.6% purity) was obtained as an off-white solid.

LCMS: RT=2.837 min. m/z=442.4 (M+1)⁺.

¹H NMR: 400 MHz CDCl₃: δ 7.90 (s, 1H), 7.63 (s, 2H), 7.06-7.01 (m, 3H), 6.74-6.71 (m, 2H), 5.89 (s, 1H), 4.78-4.70 (m, 1H), 3.83-3.80 (m, 2H), 2.93-2.80 (m, 2H), 2.50-2.44 (m, 2H), 2.09 (s, 3H), 1.96-1.86 (m, 2H), 1.77-1.58 (m, 2H), 1.00-0.97 (m, 3H).

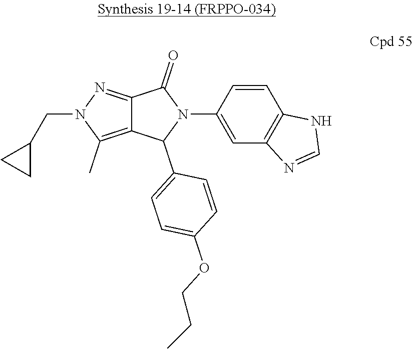 [SMILES: CCCOc1ccc(C2c3c(nn(CC4CC4)c3C)C(=O)N2c2ccc3[nH]cnc3c2)cc1]

The compound was obtained using an analogous method. Cpd 55 (6.32 mg, 16.2% yield, 98.9% purity) was obtained as an off-white solid.

LCMS: RT=2.801 min, m/z=442.3 (M+1)⁺.

¹H NMR: 400 MHz CDCl₃: δ 7.90 (s, 1H), 7.63 (s, 2H), 7.11 (s, 1H), 7.10-7.04 (m, 2H), 6.74-6.72 (m, 2H), 5.91 (s, 1H), 4.11-4.01 (m, 2H), 3.83-3.80 (m, 2H), 2.15 (s, 3H), 1.77-1.72 (m, 2H), 1.31-1.30 (m, 1H), 1.00-0.97 (m, 3H), 0.65-0.61 (m, 2H), 0.46-0.44 (m, 2H).

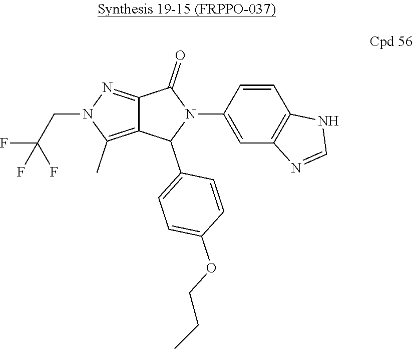 [SMILES: CCCOc1ccc(C2c3c(nn(CC(F)(F)F)c3C)C(=O)N2c2ccc3[nH]cnc3c2)cc1]

The compound was obtained using an analogous method. Cpd 56 (5.49 mg, 11.7 μmol, 70.1% yield, 100% purity) was obtained as a brown solid.

LCMS: RT=0.900 min, m/z=470.2 (M+1)⁺.

HPLC: RT=2.390 min.

¹H NMR: 400 MHz DMSO: δ 8.18 (s, 1H), 7.67 (s, 1H), 7.54-7.43 (d, J=42.4 Hz, 1H), 7.33-7.27 (d, J=24 Hz, 1H), 7.15-7.13 (d, J=8.4 Hz, 2H), 6.78-6.76 (d, J=8.8 Hz, 2H), 6.41 (s, 1H), 5.29-5.22 (m, 2H), 3.80-3.75 (m, 2H), 2.13 (s, 3H), 1.67-1.58 (m, 2H), 0.91-0.88 (m, 3H).

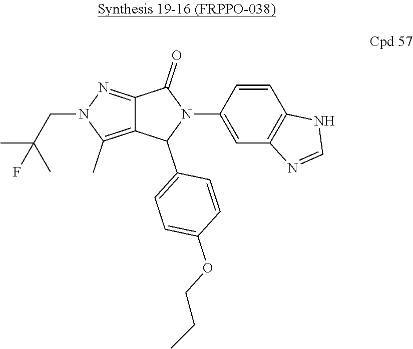 [SMILES: CCCOc1ccc(C2c3c(nn(CC(C)(C)F)c3C)C(=O)N2c2ccc3[nH]cnc3c2)cc1]

The compound was obtained using an analogous method. Cpd 57 (1 mg, 2.13 μmol, 25.2% yield, 98.1% purity) was obtained as a white solid.

LCMS: RT=0.909 min, m/z=462.3 (M+1)⁺.

HPLC: RT=2.444 min.

¹H NMR: EW14646-224-P1A 400 MHz DMSO: δ 12.4 (s, 1H), 8.16 (s, 1H), 7.67 (s, 1H), 7.52-7.27 (m, 2H), 7.14-7.12 (d, J=8.4 Hz, 2H), 6.78-6.76 (d, J=8.4 Hz, 2H), 6.37 (s, 1H), 4.43-4.36 (m, 2H), 3.81-3.77 (m, 2H), 2.07 (s, 3H), 1.66-1.60 (m, 2H), 1.39-1.24 (m, 6H), 0.92-0.88 (m, 3H).

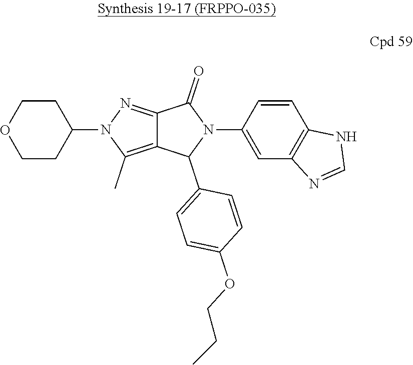 [SMILES: CCCOc1ccc(C2c3c(nn(C4CCOCC4)c3C)C(=O)N2c2ccc3[nH]cnc3c2)cc1]

The compound was obtained using an analogous method. Cpd 59 (17.0 mg, 21.7% yield, 100% purity) was obtained as an off-white solid.

LCMS: RT=2.700 min, m/z=472.3 (M+1)⁺.

¹H NMR: 400 MHz CDCl₃: δ 7.84 (s, 1H), 7.58 (s, 2H), 7.05-7.01 (m, 3H), 6.74-6.72 (m, 2H), 5.89 (s, 1H), 4.29-4.28 (m, 1H), 4.20-4.15 (m, 2H), 3.83-3.80 (m, 2H), 3.59-3.53 (m, 2H), 2.54-2.41 (m, 2H), 2.16 (s, 3H), 1.95-1.89 (m, 2H), 1.77-1.61 (m, 2H), 1.01-0.89 (m, 3H).

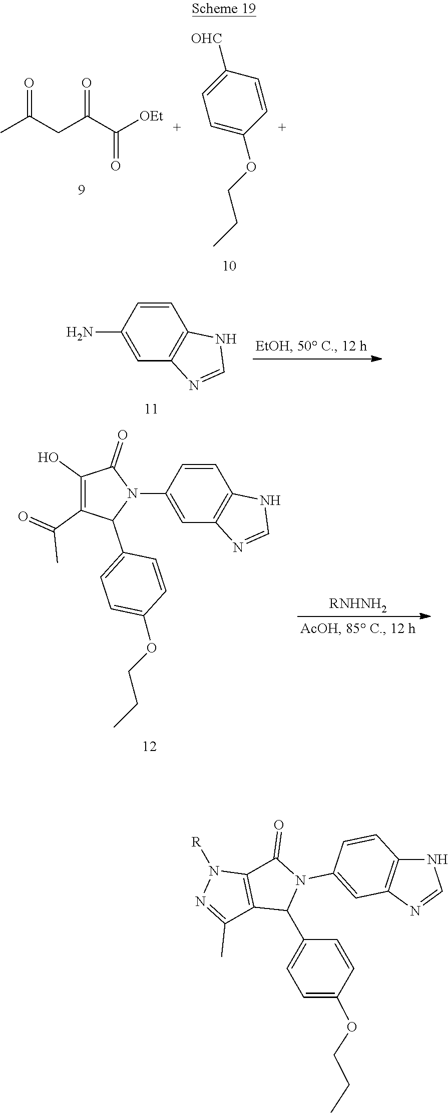

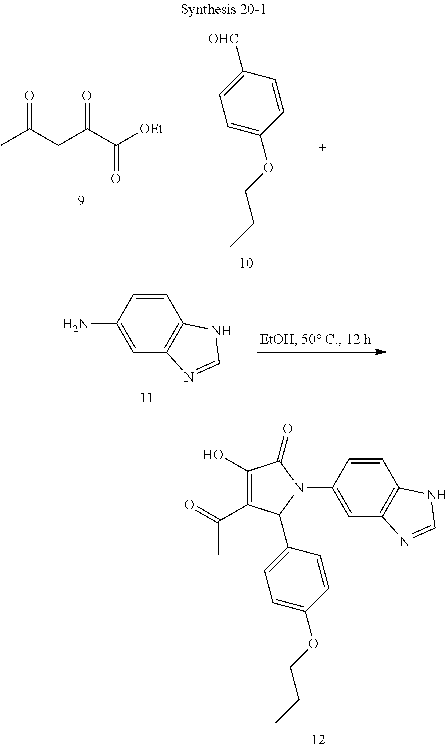 [SMILES: CCOC(=O)C(=O)CC(C)=O ; CCCOc1ccc(C=O)cc1 ; Nc1ccc2[nH]cnc2c1 ; CCCOc1ccc(C2C(C(C)=O)=C(O)C(=O)N2c2ccc3[nH]cnc3c2)cc1]

A solution of compound 10 (5.19 g, 31.6 mmol, 4.99 mL, 1.00 eq) and compound 11 (4.21 g, 31.6 mmol, 1.00 eq) in EtOH (20 mL) was stirred at 25° C. for 0.5 hour. Then compound 9 (5.00 g, 31.6 mmol, 4.50 mL, 1.00 eq) was added. The mixture was stirred at 50° C. for 12 hours. LCMS showed compound 11 was consumed and the desired mass was detected. The reaction mixture was concentrated under reduced pressure to give a residue which was purified by reversed-phase HPLC (0.1% formic acid H₂O/MeCN) to give compound 12 (800 mg, 2.04 mmol, 6.46% yield) as a yellow solid.

LCMS: RT=0.785 min. m/z=392.1 (M+1)⁺.

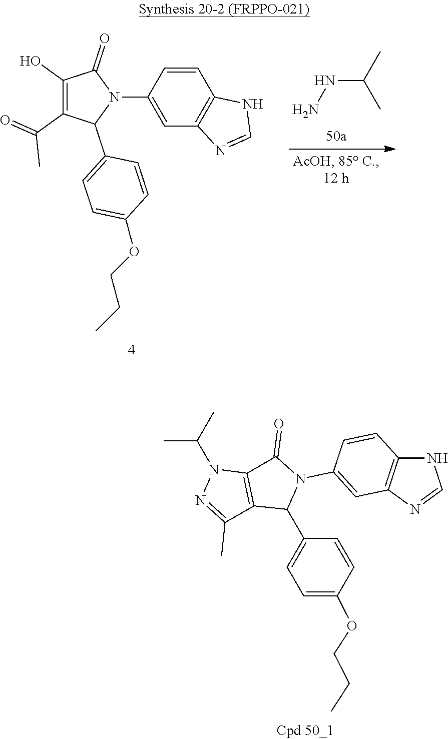

To a solution of compound 12 (100 mg, 255 μmol, 1.00 eq) in AcOH (2 mL) was added compound 50a (189 mg, 2.55 mmol, 10.0 eq). The mixture was stirred at 85° C. for 12 hours. The reaction mixture was concentrated under reduced pressure to give a residue which was purified by preparative HPLC (column Xtimate C18 150×25 mm, 5 μm; mobile phase (0.04% NH₄OH, 10 mM NH₄HCO₃) A—water, B—ACN; gradient 35-65% B, 10 min) to give Cpd 50_1 (23.52 mg, 54.2 μmol, 21.2% yield, 99.0% purity) as a light yellow solid.

LCMS: RT=0.844 min, m/z=430.2 (M+1)⁺.

HPLC: RT=1.777 min.

¹H NMR: 400 MHz CDCl₃: δ 10.0 (s, 1H), 7.83 (s, 1H), 7.55-7.53 (m, 1H), 7.14 (s, 1H), 7.05-7.02 (m, 2H), 6.75-6.72 (m, 2H), 5.83 (s, 1H), 4.96-4.88 (m, 1H), 3.86-3.78 (m, 2H), 2.07 (s, 3H), 1.79-1.77 (m, 2H), 1.76-1.72 (m, 3H), 1.66-1.64 (m, 3H), 1.01-0.97 (m, 3H).

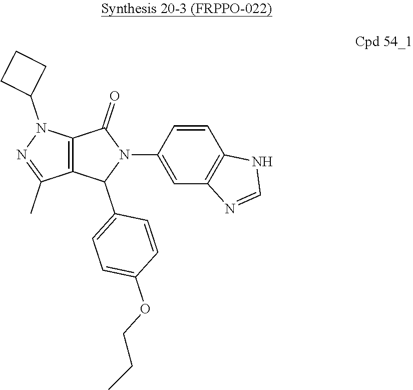 [SMILES: CCCOc1ccc(C2c3c(C)nn(C4CCC4)c3C(=O)N2c2ccc3[nH]cnc3c2)cc1]

The compound was obtained using an analogous method. Cpd 54_1 (5.72 mg, 12.6 μmol, 4.93% yield, 97.3% purity) was obtained as a light yellow solid.

LCMS: RT=0.872 min, m/z=442.3 (M+1)⁺.

HPLC: RT=1.923 min.

¹H NMR: 400 MHz CDCl₃: δ 7.99 (s, 1H), 7.63-7.54 (m, 2H), 7.26-7.25 (m, 1H), 7.08-7.06 (m, 2H), 6.77-6.74 (m, 2H), 5.86 (s, 1H), 5.21-5.12 (m, 1H), 3.85-3.82 (m, 2H), 2.91-2.86 (s, 2H), 2.56-2.53 (m, 2H), 2.09-2.08 (m, 3H), 1.97-1.95 (m, 1H), 1.88-1.86 (m, 1H), 1.79-1.76 (m, 2H), 1.03-0.98 (m, 3H).

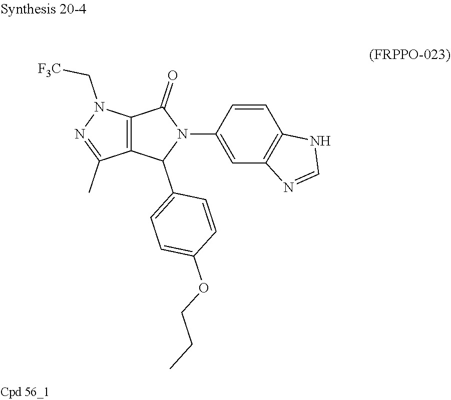 [SMILES: CCCOc1ccc(C2c3c(C)nn(CC(F)(F)F)c3C(=O)N2c2ccc3[nH]cnc3c2)cc1]

The compound was obtained using an analogous method. Cpd 56_1 (45.72 mg, 93.7 μmol, 36.7% yield, 96.3% purity) was obtained as a yellow solid.

LCMS: RT=0.838 min, m/z=470.2 (M+1)⁺.

HPLC: RT=1.769 min

¹H NMR: 400 MHz CDCl₃ 7.93 (s, 1H), 7.60-7.59 (m, 1H), 7.52-7.50 (m, 1H), 7.20-7.19 (m, 1H), 7.18-7.17 (m, 2H), 6.77-6.74 (m, 2H), 5.87 (s, 1H), 4.99-4.89 (m, 3H), 3.84-3.81 (m, 2H), 2.10 (s, 3H), 1.77-1.72 (m, 2H), 1.01-0.97 (m, 3H).

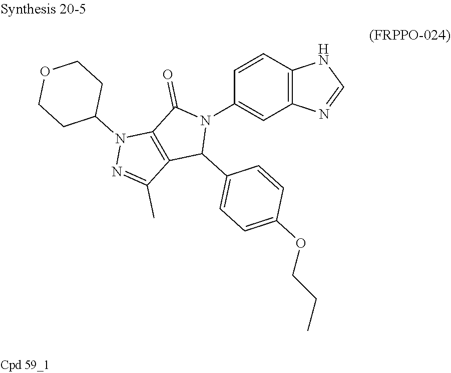 [SMILES: CCCOc1ccc(C2c3c(C)nn(C4CCOCC4)c3C(=O)N2c2ccc3[nH]cnc3c2)cc1]

The compound was obtained using an analogous method. Cpd 59_1 (23.37 mg, 48.4 μmol, 18.9% yield, 97.8% purity) was obtained as a light yellow solid.

LCMS: RT=0.802 min, m/z=472.2 (M+1)⁺.

HPLC: RT=1.791 min.

¹H NMR: 400 MHz CDCl₃: δ 7.95 (s, 1H), 7.61-7.54 (m, 1H), 7.53-7.52 (m, 1H), 7.22-7.20 (m, 1H), 7.06-7.04 (m, 2H), 6.76-6.73 (m, 2H), 5.86 (s, 1H), 4.76-4.70 (m, 1H), 4.16-4.12 (m, 2H), 3.84-3.81 (m, 2H), 3.62-3.56 (m, 2H), 2.49-2.42 (m, 2H), 2.11-2.08 (m, 5H), 1.78-1.72 (m, 2H), 1.01-0.97 (m, 3H).

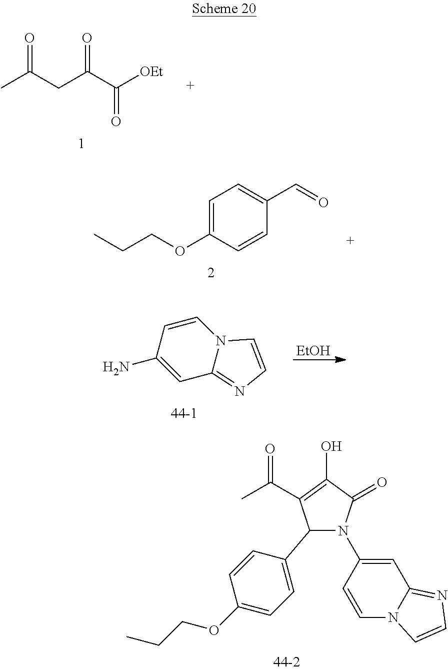 [SMILES: "EtOH" ; CCOC(=O)C(=O)CC(C)=O ; CCCOc1ccc(C=O)cc1 ; Nc1ccn2ccnc2c1 ; CCCOc1ccc(C2C(C(C)=O)=C(O)C(=O)N2c2ccn3ccnc3c2)cc1]

### Synthesis 21-1

To a solution of compound 2 (200 mg, 1.36 mmol, 1.00 eq) in EtOH (8 mL) was added compound 44_1 (181 mg, 1.36 mmol, 1.00 eq). The mixture was stirred at 25° C. for 0.5 hour, then compound 1 (215 mg, 1.36 mmol, 192 μL, 1.00 eq) was added to the mixture and the reaction was stirred at 50° C. for 2 hours. The reaction mixture concentrated under reduced pressure to give a residue. The crude product was purified by reversed-phase HPLC (0.1% formic acid H₂O/MeCN) to give 44-2 as a white solid which was used directly in the following step.

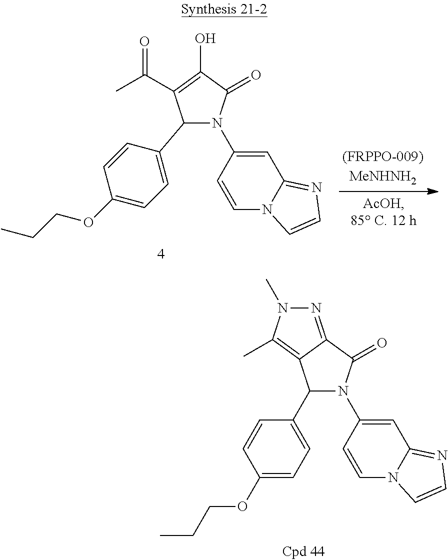 [SMILES: CCCOc1ccc(C2C(C(C)=O)=C(O)C(=O)N2c2ccn3ccnc3c2)cc1 ; CCCOc1ccc(C2c3c(nn(C)c3C)C(=O)N2c2ccn3ccnc3c2)cc1]

To a solution of compound 44_2 (33 mg, 87 μmol, 1.0 eq) in AcOH (5 mL) was added MeNHNH₂ (40 mg, 868 μmol, 46 μL, 10.1 eq). The mixture was stirred at 85° C. for 12 hours. The reaction mixture was concentrated under reduced pressure to give a residue which was purified by preparative HPLC (column Shim-pack C18 150×25, 10 μm; mobile phase (0.23% formic acid) A—water, B—ACN; gradient 17-47% B, 10 min) to give Cpd 44 (5.22 mg, 12.6 μmol, 1.97% yield, 97% purity) as a yellow solid.

LCMS: RT=0.945 min, m/z=402.4 (M+1)⁺.

HPLC: RT=2.176 min

¹H NMR: 400 MHz MeOD: δ 8.32-8.30 (d, J=8, 1H), 7.70 (s, 1H), 7.57 (s, 1H), 7.47-7.46 (m, 1H), 7.42-7.39 (m, 1H), 7.20-7.18 (m, 2H), 6.83-6.81 (d, J=8, 2H), 6.29 (s, 1H), 3.91 (s, 3H), 3.85-3.82 (m, 2H), 2.14 (m, 3H), 1.76-1.67 (m, 2H), 0.99-0.95 (m, 3H).

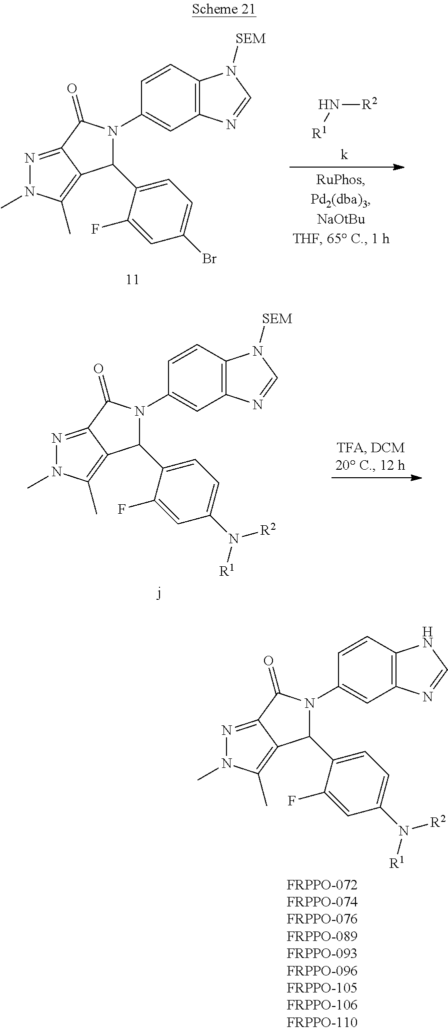

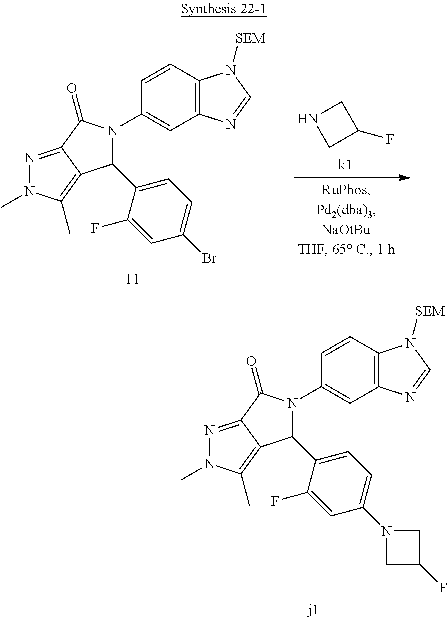 [SMILES: Cc1c2c(nn1C)C(=O)N(c1ccc3c(c1)ncn3COCC[Si](C)(C)C)C2c1ccc(Br)cc1F ; FC1CNC1 ; Cc1c2c(nn1C)C(=O)N(c1ccc3c(c1)ncn3COCC[Si](C)(C)C)C2c1ccc(N2CC(F)C2)cc1F]

A mixture of compound 11 (0.500 g, 876 μmol, 1.00 eq), compound k1 (195 mg, 1.75 mmol, 2.00 eq, HCl), Pd₂(dba)₃ (80 mg, 87.6 μmol, 0.100 eq), RuPhos (82 mg, 175 μmol, 0.200 eq) and NaOtBu (336 mg, 3.51 mmol, 4.00 eq) in THF (10 mL) was degassed and purged with N₂ for 3 times, and then the mixture was stirred at 65° C. for 1 h under N₂ atmosphere. The mixture was concentrated and purified by reversed-phase HPLC (0.1% FA condition in acetonitrile) to give compound j1 (0.335 g crude) as a yellow oil, which was used in the next step directly without further purification.

LCMS: RT=1.043 min, m/z=565.4 (M+1)⁺

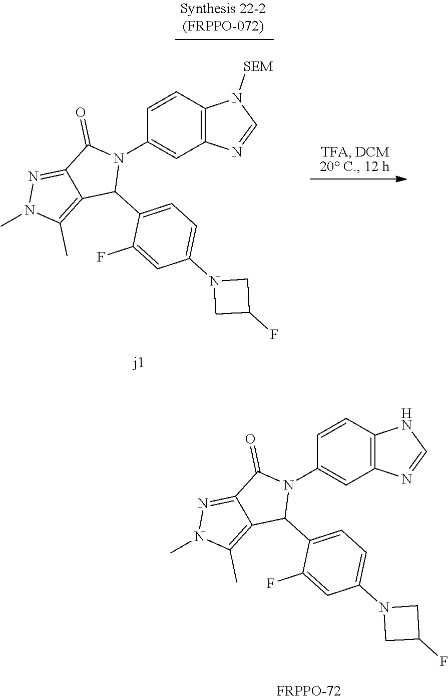 [SMILES: Cc1c2c(nn1C)C(=O)N(c1ccc3c(c1)ncn3COCC[Si](C)(C)C)C2c1ccc(N2CC(F)C2)cc1F ; "TFA, DCM20° C., 12 h" ; Cc1c2c(nn1C)C(=O)N(c1ccc3[nH]cnc3c1)C2c1ccc(N2CC(F)C2)cc1F]

To the solution compound j1 (0.335 g, 593 μmol, 1.00 eq) in DCM (10.0 mL) was added TFA (6.16 g, 54.0 mmol, 4 mL, 91.1 eq) stirred at 25° C. for 16 hrs. The reaction solution was partitioned between CH₂Cl₂ (20 mL) and saturated NaHCO₃ (aq) was added till pH=8. The organic layer was separated, dried over Na₂SO₄ and evaporated to give a residue. The residue was purified by prep-HPLC (column: Waters Xbridge 150×25 mm, 5 μm; mobile phase: (0.05% NH₄OH) A—water, B—ACN; gradient 27%-47% B, 10 min) to give compound FRPPO-072 (6.79 mg, 22.5 μmol, 3.79% yield, 97.7% purity) as off-white solid.

LCMS: RT=0.858 min, m/z=435.3 (M+1)⁺.

HPLC: RT=1.300 min.

¹HNMR: 400 MHz DMSO δ 8.14 (s, 1H), 7.64 (s, 1H), 7.49 (d, J=8.68 Hz, 1H), 7.19-7.29 (m, 1H), 6.93-7.04 (m, 1H), 6.42 (s, 1H), 6.14 (d, J=1.47 Hz, 2H), 5.27-5.53 (m, 1H), 3.96-4.18 (m, 4H), 3.87 (s, 3H), 2.11 (s, 3H).

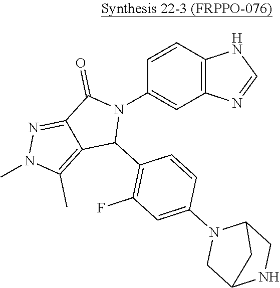 [SMILES: Cc1c2c(nn1C)C(=O)N(c1ccc3[nH]cnc3c1)C2c1ccc(N2CC3CC2CN3)cc1F]

The compound was obtained using an analogous method starting with tert-butyl 2,5-diazabicyclo[2.2.1]heptane-2-carboxylate. The BOC group was removed concomitant with the SEM group with TFA in DCM. FRPPO-076 (6.69 mg, 13.5 μmol, 3.95% yield, 92.0% purity) was obtained as an off-white solid.

LCMS: RT=0.650 min, m/z=458.3 (M+1)⁺.

HPLC: RT=1.059 min.

¹HNMR: 400 MHz DMSO δ 8.16-8.13 (m, 1H), 7.56-7.51 (m, 1H), 7.25-7.23 (m, 1H), 7.17-7.14 (m, 1H), 6.45-6.44 (m, 1H), 6.43-6.27 (m, 2H), 6.25-6.13 (m, 1H), 4.25 (s, 1H), 3.83 (s, 3H), 3.54 (s, 1H), 3.45-3.36 (m, 2H), 2.82-2.71 (m, 2H), 2.26 (s, 3H), 1.79-1.54 (m, 2H), 0.99 (d, J=6.0 Hz, 1H).

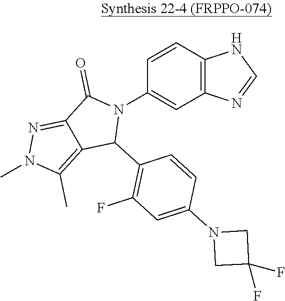 [SMILES: Cc1c2c(nn1C)C(=O)N(c1ccc3[nH]cnc3c1)C2c1ccc(N2CC(F)(F)C2)cc1F]

FRPPO-074 (14.75 mg, 30.7 μmol, 3.58% yield, 94.2% purity) was obtained using an analogous method as an off-white solid.

LCMS: RT=0.792 min, m/z=453.2 (M+1)⁺.

HPLC: RT=1.577 min.

¹HNMR: 400 MHz DMSO δ 12.4 (s, 1H), 8.17 (s, 1H), 7.66 (d, J=1.6 Hz, 1H), 7.59-7.39 (m, 1H), 7.38-7.20 (m, 1H), 7.10-6.93 (m, 1H), 6.47 (s, 1H), 6.35-6.14 (m, 2H), 4.19 (t, J=12.4 Hz, 4H), 3.86 (s, 3H), 2.11 (s, 3H).

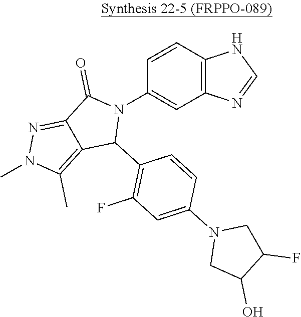 [SMILES: Cc1c2c(nn1C)C(=O)N(c1ccc3[nH]cnc3c1)C2c1ccc(N2CC(O)C(F)C2)cc1F]

FRPPO-089 (1.85 mg, 3.69 μmol, 0.080% yield, 92.6% purity) was obtained using an analogous method as a yellow solid.

LCMS: RT=0.739 min, m/z=461.4 (M+1)⁺.

HPLC: RT=1.586 min.

¹HNMR: 400 MHz DMSO δ 8.15 (s, 1H), 7.65 (s, 1H), 7.49 (d, J=7.6 Hz, 1H), 7.29 (d, J=8.8 Hz, 1H), 6.96 (t, J=8.8 Hz, 1H), 6.39 (s, 1H), 6.22-6.19 (m, 2H), 5.05-4.92 (m, 1H), 4.28-4.25 (m, 1H), 3.86 (s, 3H), 3.52-3.34 (m, 3H), 3.11-3.07 (m, 1H), 2.11 (s, 3H).

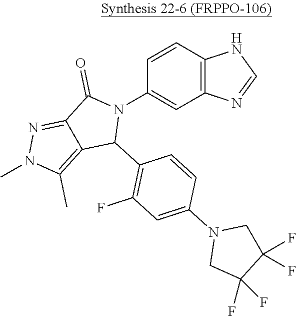 [SMILES: Cc1c2c(nn1C)C(=O)N(c1ccc3[nH]cnc3c1)C2c1ccc(N2CC(F)(F)C(F)(F)C2)cc1F]

FRPPO-106 (7.11 mg, 39.69 μmol, 12.55% yield, 99.7% purity) was obtained using an analogous method as an off-white solid.

LCMS: product Rt=0.823 min, m/z=503.1 (M+1)⁺.

HPLC: product Rt=1.547 min, purity: 99.7%.

¹HNMR: 400 MHz, DMSO-d₆ δ ppm 12.42 (s, 1H), 8.17 (s, 1H), 7.67 (s, 1H), 7.67-7.56 (m, 1H), 7.54-7.45 (m, 1H), 7.09-7.07 (m, 1H), 6.50-6.47 (m, 1H), 6.44-6.41 (m, 1H), 6.37-6.35 (m, 1H), 4.01-3.91 (m, 4H), 3.87 (s, 3H), 2.12 (s, 3H).

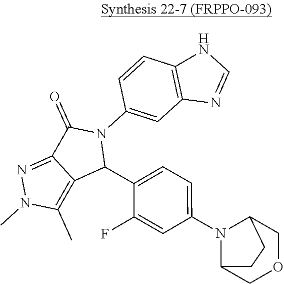 [SMILES: Cc1c2c(nn1C)C(=O)N(c1ccc3[nH]cnc3c1)C2c1ccc(N2C3CCC2COC3)cc1F]

FRPPO-093 (1.21 mg, 2.52 μmol, 0.08% yield, 98.6% purity) was obtained using an analogous method as a white solid.

LCMS: RT=0.862 min. m/z=473.3 (M+1)⁺.

HPLC: RT=1.498 min.

¹HNMR: 400 MHz DMSO δ 12.42 (br d, J=4.4 Hz, 1H), 8.18 (d, J=4.4 Hz, 1H), 7.67 (d, J=1.6 Hz, 1H), 7.57-7.41 (m, 1H), 7.37-7.20 (m, 1H), 6.96-6.95 (m, 1H), 6.56-6.41 (m, 3H), 4.05 (s, 2H), 3.86 (s, 3H), 3.55-3.51 (m, 2H), 3.36-3.34 (m, 2H), 2.13 (s, 3H), 1.86-1.76 (m, 4H).

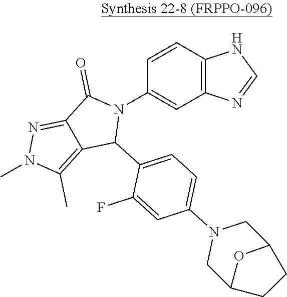 [SMILES: Cc1c2c(nn1C)C(=O)N(c1ccc3[nH]cnc3c1)C2c1ccc(N2CC3CCC(C2)O3)cc1F]

FRPPO-096 (4.92 mg, 9.67 μmol, 2.33% yield, 92.9% purity) was obtained using an analogous method as an off-white solid.

LCMS: ERT=0.785 min, m/z=473.3 (M+1)⁺.

HPLC: RT=1.532 min.

¹HNMR: 400 MH DMSO δ 8.16 (s, 1H), 7.67 (d, J=1.2 Hz, 1H), 7.50 (d, J=8.8 Hz, 1H), 7.29 (dd, J=1.6, 8.7 Hz, 1H), 7.01-6.92 (m, 1H), 6.51-6.45 (m, 3H), 4.34 (br s, 2H), 3.86 (s, 3H), 3.27-3.25 (m, 4H), 2.74-2.70 (m, 2H), 2.12 (s, 3H), 1.76-1.69 (m, 4H).

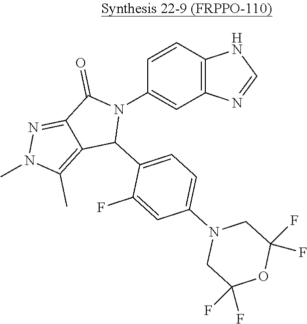 [SMILES: Cc1c2c(nn1C)C(=O)N(c1ccc3[nH]cnc3c1)C2c1ccc(N2CC(F)(F)OC(F)(F)C2)cc1F]

FRPPO-110 (82.74 mg, 159 μmol, 34.3% yield, 99.5% purity) was obtained using an analogous method as an off-white solid.

LCMS: RT=0.908 min, m/z=519.3 (M+1)⁺.

HPLC: RT=2.819 min.

¹HNMR: 400 MHz DMSO δ 12.43 (brs, 1H), 8.17 (s, 1H), 7.69 (d, J=1.6 Hz, 1H), 7.68-7.56m, 1H), 7.36-7.19 (m, 1H), 7.10 (s, 1H), 6.79-6.77 (m, 2H), 6.51 (d, J=8.4 Hz, 1H), 3.97 (t, J=8.4 Hz, 4H), 3.86 (s, 3H), 2.12 (s, 3H).

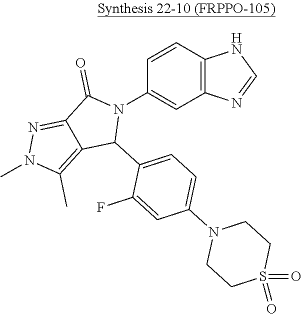 [SMILES: Cc1c2c(nn1C)C(=O)N(c1ccc3[nH]cnc3c1)C2c1ccc(N2CCS(=O)(=O)CC2)cc1F]

FRPPO-105 (23.62 mg, 46.7 μmol, 14.6% yield, 97.7% purity) was obtained using an analogous method as a yellow solid.

LCMS: RT=0.803 min, m/z=495.3 (M+1)⁺.

HPLC: RT=1.944 min.

¹HNMR: 400 MHz DMSO δ 12.42 (s, 1H), 8.17 (d, J=4.0 Hz, 1H), 7.68 (d, J=1.6 Hz, 1H), 7.56-7.54 (m, 1H), 7.43-7.41 (m, 1H), 7.03 (q, J=8.4 Hz, 1H), 6.77-6.65 (m, 2H), 6.48 (d, J=10.4 Hz, 1H), 3.86 (s, 3H), 3.68 (br s, 4H), 3.02 (br s, 4H), 2.12 (s, 3H).

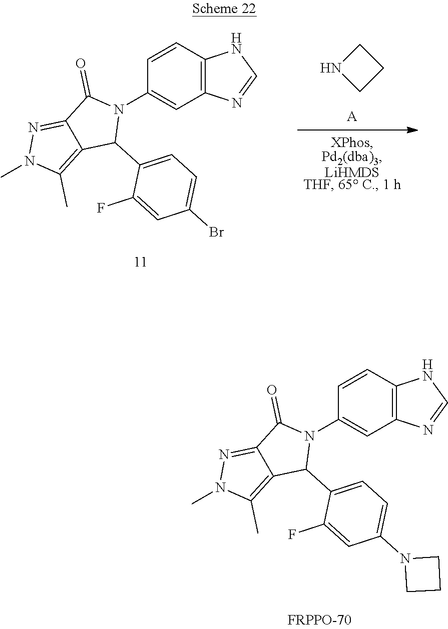

### Synthesis 23-1

### FRPPO-070

To a solution of 11 (1.00 g, 2.27 mmol, 1.00 eq), compound A (194 mg, 3.41 mmol, 230 uL, 1.50 eq), Pd₂(dba)₃ (104 mg, 114 μmol, 0.050 eq), XPhos (108 mg, 227 μmol, 0.100 eq) in THF (12 mL), was added LiHMDS (1 M, 11.4 mL, 5.00 eq). The reaction was concentrated to give a crude, which was purified by prep-HPLC (column: Waters Xbridge 150×25 mm, 5 μm; mobile phase: (0.1% TFA) A—water, B—ACN; gradient 1%-30% B, 7 min) to give FRPPO-70 (32.0 mg, 75.2 μmol, 20.9% yield, 97.7% purity) as a light yellow solid.

LCMS: RT=0.856 min, m/z=417.1 [M+1]⁺.

HPLC: RT=2.482 min, 97.7% purity.

¹HNMR: 400 MHz DMSO δ 10.56 (s, 1H), 7.91 (s, 1H), 7.78-7.53 (m, 1H), 7.27-7.14 (m, 1H), 6.80-6.76 (m, 1H), 6.29 (s, 1H), 6.00-5.94 (m, 2H), 3.93 (s, 3H), 3.79 (t, J=7.3 Hz, 4H), 2.35-2.26 (m, 2H), 1.65 (s, 3H).

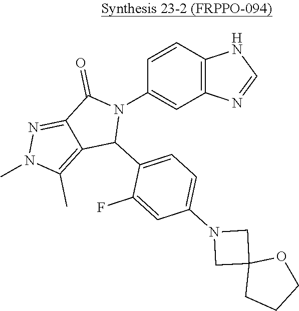 [SMILES: Cc1c2c(nn1C)C(=O)N(c1ccc3[nH]cnc3c1)C2c1ccc(N2CC3(CCCO3)C2)cc1F]

FRPPO-094 (93.23 mg, 185 μmol, 58.1% yield, 93.5% purity) was obtained using an analogous method as a yellow solid.

LCMS: RT=0.877 min, m/z=473.3 (M+1)⁺.

HPLC: RT=1.992 min.

¹HNMR: 400 MHz DMSO δ 12.42 (br d, J=4.0 Hz, 1H), 8.18 (d, J=4.4 Hz, 1H), 7.65-7.64 (m, 1H), 7.55-7.53 (m, 1H), 7.44-7.42 (m, 1H), 6.96-6.94 (m, 1H), 6.43-6.41 (m, 1H), 6.10-6.06 (m, 2H), 3.86 (s, 3H), 3.77-3.66 (m, 6H), 2.11 (s, 3H), 2.03-1.99 (m, 2H), 1.85-1.80 (m, 2H).

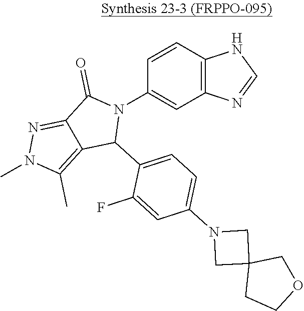 [SMILES: Cc1c2c(nn1C)C(=O)N(c1ccc3[nH]cnc3c1)C2c1ccc(N2CC3(CCOC3)C2)cc1F]

FRPPO-095 (0.57 mg, 1.19 μmol, 0.06% yield, 98.4% purity) was obtained using an analogous method as a an off-white solid.

LCMS: RT=0.869 min, m/z=473.3 (M+1)⁺.

HPLC: RT=1.828 min.

¹HNMR: 400 MHz DMSO δ 12.42 (s, 1H), 8.18 (s, 1H), 7.64 (s, 1H), 7.56-7.45 (m, 2H), 6.97-6.96 (m, 1H), 6.44-7.42 (m, 1H), 6.10-6.07 (m, 2H), 3.89 (s, 3H), 3.73-3.66 (m, 8H), 3.49-3.57 (m, 2H), 2.12-2.04 (m, 5H).

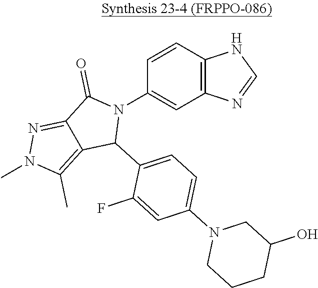 [SMILES: Cc1c2c(nn1C)C(=O)N(c1ccc3[nH]cnc3c1)C2c1ccc(N2CCCC(O)C2)cc1F]

FRPPO-086 (106.07 mg, 210 μmol, 48.3% yield, 91.1% purity) was obtained using an analogous method as a brown solid.

LCMS: RT=0.739 min, m/z=461.4 (M+1)⁺.

HPLC: RT=1.586 min.

¹HNMR: 400 MHz DMSO δ 8.17 (s, 1H), 7.66 (s, 1H), 7.49 (br d, J=7.6 Hz, 1H), 7.29 (br d, J=8.8 Hz, 1H), 6.96 (br t, J=8.8 Hz, 1H), 6.63-6.48 (m, 2H), 6.43 (s, 1H), 4.78 (br s, 1H), 3.86 (s, 3H), 3.56-3.44 (m, 4H), 2.66-2.60 (m, 1H), 2.11 (s, 3H), 1.81-1.63 (m, 2H), 1.24-1.21 (m, 2H).

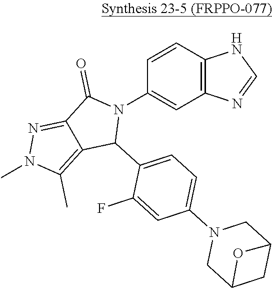 [SMILES: Cc1c2c(nn1C)C(=O)N(c1ccc3[nH]cnc3c1)C2c1ccc(N2CC3CC(C2)O3)cc1F]

FRPPO-077 (2.82 mg, 6.10 μmol, 1.40% yield, 99.1% purity) was obtained using an analogous method as an off-white solid.

LCMS: RT=0.858 min, m/z=459.3 (M+1)⁺.

HPLC: RT=2.169 min.

¹HNMR: 400 MHz DMSO δ 12.51-12.30 (m, 1H), 8.17 (s, 1H), 7.66 (d, J=1.6 Hz, 1H), 7.55-7.39 (m, 1H), 7.38-7.20 (m, 1H), 7.08-6.93 (m, 1H), 6.45 (s, 1H), 6.42-6.28 (m, 2H), 4.62 (br d, J=6.4 Hz, 2H), 3.87 (s, 3H), 3.57-3.50 (m, 4H), 2.12 (s, 3H), 2.03-1.99 (m, 1H), 1.81-1.78 (m, 1H).

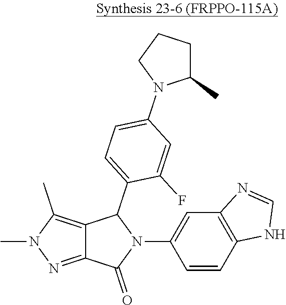 [SMILES: Cc1c2c(nn1C)C(=O)N(c1ccc3[nH]cnc3c1)C2c1ccc(N2CCC[C@H]2C)cc1F]

FRPPO-115A (5.40 mg, 11.7 μmol, 3.46% yield, 96.0% purity) was obtained using an analogous method as a yellow solid.

LCMS: RT=0.960 min, m/z=445.3 (M+1)⁺.

HPLC: RT=2.662 min.

¹HNMR: 400 MHz DMSO δ 12.36 (s, 1H), 8.16 (s, 1H), 7.64 (s, 1H), 7.54-7.29 (m, 2H), 6.91-6.89 (m, 1H), 6.40-6.38 (m, 1H), 6.19-6.11 (m, 2H), 3.87 (s, 3H), 3.72-3.69 (m, 1H), 3.23-3.20 (m, 1H), 3.00-2.88 (m, 1H), 2.10 (d, J=2.0 Hz, 3H), 1.93-1.84 (m, 3H), 1.61-1.56 (m, 1H), 0.99-0.96 (m, 3H).

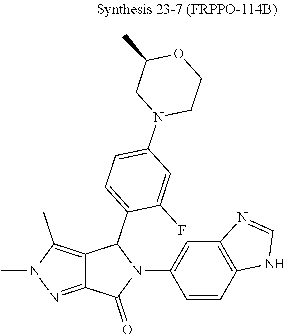 [SMILES: Cc1c2c(nn1C)C(=O)N(c1ccc3[nH]cnc3c1)C2c1ccc(N2CCO[C@H](C)C2)cc1F]

FRPPO-114B (45.21 mg, 89.3 μmol, 20.6% yield, 91.0% purity) was obtained using an analogous method as a white solid.

LCMS: RT=0.825 min, m/z=461.1 (M+1)⁺.

HPLC: RT=2.356 min.

¹HNMR: 400 MHz DMSO δ 12.43 (s, 1H), 8.18 (d, J=3.6 Hz, 1H), 7.66 (s, 1H), 7.56-7.42 (m, 1H), 7.35-7.19 (m, 1H), 7.01-6.99 (m, 1H), 6.63-6.58 (m, 2H), 6.46 (d, J=11.6 Hz, 1H), 3.86-3.83 (m, 1H), 3.51-3.48 (m, 3H), 3.45-3.39 (m, 1H), 2.62-2.56 (m, 1H), 2.27-2.21 (m, 1H), 2.11 (s, 3H), 1.07 (d, J=6.0 Hz, 3H).

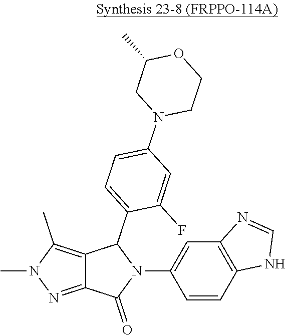 [SMILES: Cc1c2c(nn1C)C(=O)N(c1ccc3[nH]cnc3c1)C2c1ccc(N2CCO[C@@H](C)C2)cc1F]

FRPPO-114A (3.12 mg, 24 mol, 1.92% yield, 92.1% purity) was obtained using an analogous method as an off-white solid.

LCMS: RT=0.776 min, m/z=461.4 (M+1)⁺.

HPLC: RT=1.832 min.

¹HNMR: 400 MHz DMSO δ 8.17 (s, 1H), 7.66 (d, J=1.6 Hz, 1H), 7.50-7.46 (m, 1H), 7.28 (d, J=8.8 Hz, 1H), 7.00 (t, J=9.2 Hz, 1H), 6.63-6.58 (m, 2H), 6.45 (s, 1H), 3.89-3.79 (m, 5H), 3.51-3.45 (m, 4H), 2.28-2.22 (m, 1H), 2.11 (s, 3H), 1.07 (d, J=6.0 Hz, 3H).

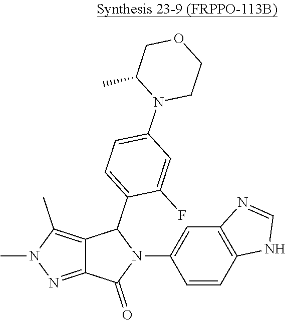 [SMILES: Cc1c2c(nn1C)C(=O)N(c1ccc3[nH]cnc3c1)C2c1ccc(N2CCOC[C@H]2C)cc1F]

FRPPO-113B (130.28 mg, 257 μmol, 59.3% yield, 91.0% purity) was obtained using an analogous method as a yellow solid.

LCMS: RT=0.803 min, m/z=461.3 (M+1)⁺.

HPLC: RT=2.318 min.

¹HNMR: 400 MHz DMSO δ 8.17 (s, 1H), 7.66 (s, 1H), 7.49 (d, J=8.8 Hz, 1H), 7.28 (dd, J=1.6, 8.6 Hz, 1H), 6.98 (t, J=8.8 Hz, 1H), 6.54-6.53 (m, 2H), 6.43 (s, 1H), 3.86 (s, 3H), 3.81-3.63 (m, 2H), 3.60-3.53 (m, 2H), 3.18-3.15 (m, 2H), 2.91-2.88 (m, 1H), 2.11 (s, 3H), 0.97-0.91 (m, 3H).

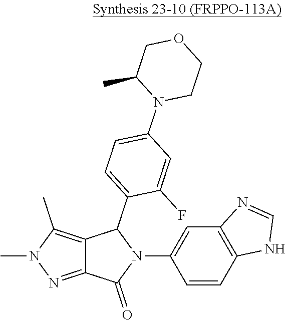 [SMILES: Cc1c2c(nn1C)C(=O)N(c1ccc3[nH]cnc3c1)C2c1ccc(N2CCOC[C@@H]2C)cc1F]

FRPPO-113A (5.43 mg, 11.7 μmol, 5.38% yield, 99.0% purity) was obtained using an analogous method as a yellow solid.

LCMS: RT=0.857 min, m/z=461.4 (M+1)⁺.

HPLC: RT=2.308 min.

¹H NMR: 400 MHz DMSO δ 12.41 (s, 1H), 8.18 (s, 1H), 7.67 (s, 1H), 7.56-7.54 (m, 1H), 7.43-7.35 (m, 1H), 7.00-6.98 (m, 1H), 6.55-6.52 (m, 2H), 6.46-6.43 (s, 1H), 3.87 (s, 3H), 3.82-3.64 (m, 2H), 3.61-3.57 (m, 3H), 3.44-3.41 (m, 2H), 2.12 (s, 3H), 0.94-0.92 (m, 3H).

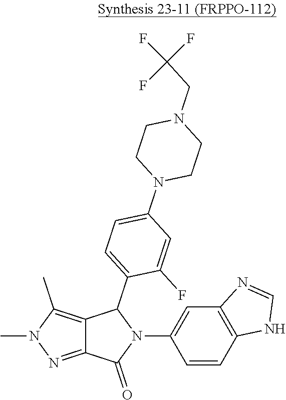 [SMILES: Cc1c2c(nn1C)C(=O)N(c1ccc3[nH]cnc3c1)C2c1ccc(N2CCN(CC(F)(F)F)CC2)cc1F]

FRPPO-112 (252.02 mg, 461 μmol, 60.8% yield, 96.5% purity) was obtained using an analogous method as a yellow solid.

LCMS: RT=0.865 min, m/z=528.3 (M+1)⁺.

HPLC: RT=2.813 min.

¹HNMR: 400 MHz DMSO δ 12.39 (m, 1H), 8.17 (s, 1H), 7.66-7.50 (m, 2H), 7.50 (s, 1H), 6.99 (t, J=9.2 Hz, 1H), 6.61-6.57 (m, 2H), 6.45 (s, 1H), 3.86 (s, 3H), 3.17 (q, J=10.4 Hz, 2H), 3.08-3.06 (m, 4H), 2.66-2.63 (m, 4H), 2.11 (s, 3H).

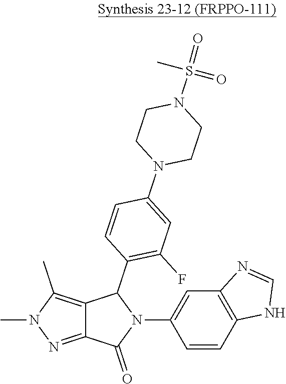 [SMILES: Cc1c2c(nn1C)C(=O)N(c1ccc3[nH]cnc3c1)C2c1ccc(N2CCN(S(C)(=O)=O)CC2)cc1F]

FRPPO-111 (4.18 mg, 7.81 μmol, 4.09% yield, 97.8% purity) was obtained using an analogous method as an off-white solid.

LCMS: RT=0.846 min, m/z=524.3 (M+H)⁺.

HPLC: RT=2.140 min, 97.8% purity.

¹HNMR: 400 MHz DMSO δ 12.42 (brs, 1H), 8.19-8.17 (m, 1H), 7.68-7.57 (m, 1H), 7.55-7.37 (m, 1H), 7.02 (s, 1H), 7.09-7.00 (m, 1H), 6.69-6.63 (m, 2H), 6.50-6.47 (m, 1H), 3.87 (s, 3H), 3.20-3.12 (m, 8H), 2.88 (s, 3H), 2.11 (s, 3H).

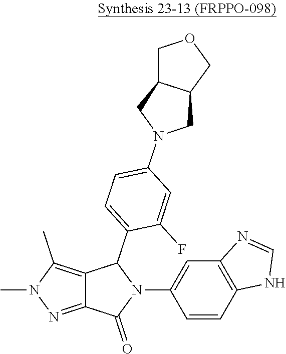 [SMILES: Cc1c2c(nn1C)C(=O)N(c1ccc3[nH]cnc3c1)C2c1ccc(N2C[C@H]3COC[C@H]3C2)cc1F]

FRPPO-098 (7.21 mg, 14.8 μmol, 6.98% yield, 96.8% purity) was obtained using an analogous method as a light yellow solid.

LCMS: RT=0.797 min, m/z=473.3 (M+H)⁺.

HPLC: RT=2.308 min.

¹HNMR: 400 MHz DMSO δ 12.42 (d, J=0.8 Hz, 1H), 8.17 (s, 1H), 7.65 (d, J=1.6 Hz, 1H), 7.54-7.45 (m, 1H), 7.42-7.26 (m, 1H), 6.95 (br t, J=6.4 Hz, 1H), 6.43-6.42 (m, 1H), 6.26-6.22 (m, 2H), 3.87 (s, 3H), 3.81-3.77 (m, 2H), 3.47-3.45 (m, 2H), 3.09-3.05 (m, 2H), 2.92 (br s, 2H), 2.12 (s, 3H).

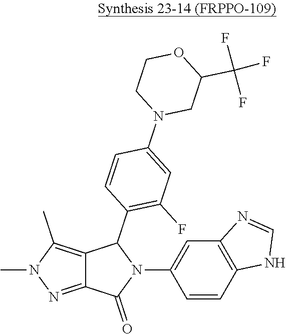 [SMILES: Cc1c2c(nn1C)C(=O)N(c1ccc3[nH]cnc3c1)C2c1ccc(N2CCOC(C(F)(F)F)C2)cc1F]

FRPPO-109 (4.16 mg, 7.33 μmol, 3.77% yield, 90.7% purity) was obtained using an analogous method as an off-white solid.

LCMS: RT=0.923 min, m/z=515.3 (M+H)⁺.

HPLC: RT=2.763 min, 90.7% purity.

¹HNMR: 400 MHz DMSO δ 12.43 (s, 1H), 8.18 (d, J=4 Hz, 1H), 7.68 (d, J=1.6 Hz, 1H), 7.67-7.56 (m, 1H), 7.54-7.43 (m, 1H), 7.06-7.04 (m, 1H), 6.77-6.67 (m, 2H), 6.49 (d, J=12 Hz, 1H), 4.23-4.20 (m, 1H), 4.02-3.99 (m, 2H), 3.90-3.87 (m, 3H), 3.67-3.64 (m, 2H), 2.76-2.75 (m, 2H), 2.12 (s, 3H).

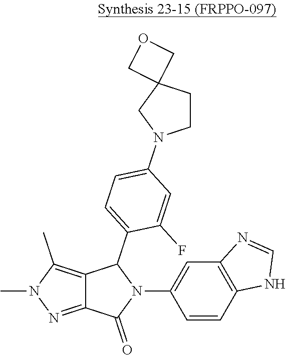 [SMILES: Cc1c2c(nn1C)C(=O)N(c1ccc3[nH]cnc3c1)C2c1ccc(N2CCC3(COC3)C2)cc1F]

FRPPO-097 (4.61 mg, 9.29 μmol, 4.39% yield, 95.2% purity) was obtained using an analogous method as an off-white solid.

LCMS: RT=0.796 min, m/z=473.3 (M+H)⁺.

HPLC: RT=2.267 min.

¹HNMR: 400 MHz DMSO δ 12.42 (s, 1H), 8.18 (d, J=4 Hz, 1H), 7.64 (d, J=0.8 Hz, 1H), 7.55-7.42 (m, 1H), 7.26-7.23 (m, 1H), 6.95-6.93 (m, 1H), 6.42 (d, J=12 Hz, 1H), 6.19-6.13 (m, 2H), 4.52-4.49 (m, 2H), 4.45-4.44 (m, 2H), 3.87 (s, 3H), 3.45-3.37 (m, 2H), 3.14-3.11 (m, 2H), 2.18-2.12 (m, 2H), 2.11 (s, 3H).

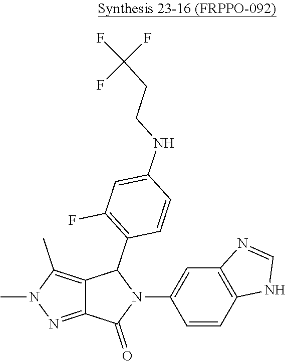 [SMILES: Cc1c2c(nn1C)C(=O)N(c1ccc3[nH]cnc3c1)C2c1ccc(NCCC(F)(F)F)cc1F]

FRPPO-092 (0.37 mg, 0.074 μmol, 0.04% yield, 94.1% purity) was obtained using an analogous method as an off-white solid.

LCMS: RT=0.841 min, m/z=473.3 (M+H)⁺.

HPLC: RT=2.634 min.

¹HNMR: 400 MHz DMSO δ 12.42-12.41 (m, 1H), 8.18-8.17 (m, 1H), 7.65 (d, J=1.6 Hz, 1H), 7.54-7.43 (m, 1H), 7.33-7.22 (m, 1H), 6.88-6.87 (m, 1H), 6.40-6.38 (m, 1H), 6.26-6.21 (m, 2H), 6.07-6.06 (m, 1H), 3.87 (s, 3H), 3.18-3.14 (m, 2H), 2.45-2.39 (m, 2H), 2.12 (s, 3H).

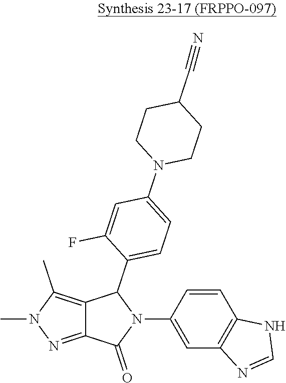 [SMILES: Cc1c2c(nn1C)C(=O)N(c1ccc3[nH]cnc3c1)C2c1ccc(N2CCC(C#N)CC2)cc1F]

FRPPO-091 (4.18 mg, 7.81 μmol, 4.09% yield, 97.8% purity) was obtained using an analogous method as an off-white solid.

LCMS: RT=0.846 min, m/z=524.3 (M+H)⁺.

HPLC: RT=2.140 min.

¹HNMR: EW18244-4-P1A 400 MHz DMSO δ 12.43 (s, 1H), 8.18 (s, 1H), 7.67 (s, 1H), 7.56-7.54 (m, 1H), 7.45-7.36 (m, 1H), 7.00-6.99 (m, 1H), 6.65-6.62 (m, 2H), 6.60-6.45 (m, 1H), 3.87 (s, 3H), 3.31-3.29 (m, 2H), 3.01-2.96 (m, 3H), 2.12 (s, 3H), 1.89-1.85 (m, 2H), 1.71-1.66 (m, 2H).

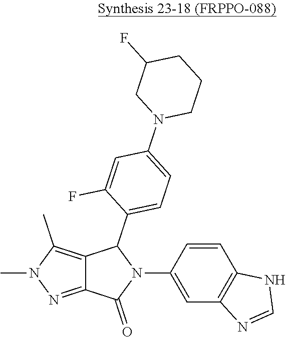 [SMILES: Cc1c2c(nn1C)C(=O)N(c1ccc3[nH]cnc3c1)C2c1ccc(N2CCCC(F)C2)cc1F]

FRPPO-088 (9.80 mg, 19.4 μmol, 8.98% yield, 91.7% purity was obtained using an analogous method as a light yellow solid.

LCMS: RT=0.831 min, m/z=463.2 (M+H)⁺.

HPLC: RT=1.924 min.

¹HNMR: 400 MHz DMSO δ 12.44 (br s, 1H), 8.18 (s, 1H), 7.67 (s, 1H), 7.49 (s, 1H), 7.30 (s, 1H), 6.98 (br t, J=9.6 Hz, 1H), 6.64-6.59 (m, 2H), 6.46 (s, 1H), 4.76-4.63 (m, 1H), 3.87 (s, 3H), 3.28-3.26 (m, 2H), 3.14-3.04 (m, 2H), 2.12 (s, 3H), 1.84 (br s, 1H), 1.69-1.68 (m, 2H), 1.48 (br s, 1H).

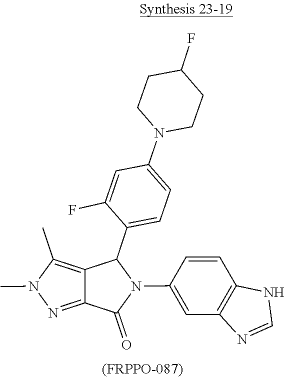 [SMILES: Cc1c2c(nn1C)C(=O)N(c1ccc3[nH]cnc3c1)C2c1ccc(N2CCC(F)CC2)cc1F]

FRPPO-087 (32.02 mg, 67.2 μmol, 31.1% yield, 97.1% purity) was obtained using an analogous method as a light yellow solid.

LCMS: RT=0.832 min, m/z=463.3 (M+H)⁺.

HPLC: RT=2.551 min.

¹HNMR: 400 MHz DMSO δ 8.18 (s, 1H), 7.67 (d, J=1.6 Hz, 1H), 7.51-7.45 (m, 1H), 7.30 (br d, J=8 Hz, 1H), 6.99 (t, J=8 Hz, 1H), 6.66-6.60 (m, 2H), 6.46 (s, 1H), 4.85-4.70 (m, 1H), 3.87 (s, 3H), 3.27-3.23 (m, 2H), 3.11-3.07 (m, 2H), 2.12 (s, 3H), 1.86-1.81 (m, 2H), 1.69-1.65 (m, 2H).

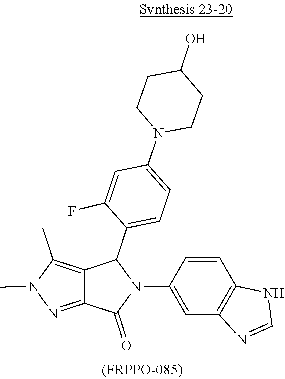 [SMILES: Cc1c2c(nn1C)C(=O)N(c1ccc3[nH]cnc3c1)C2c1ccc(N2CCC(O)CC2)cc1F]

FRPPO-085 (14.18 mg, 30.8 μmol, 9.45% yield, 98.2% purity) was obtained using an analogous method as an off-white solid.

LCMS: RT=0.825 min, m/z=461.3 (M+1)⁺.

HPLC: RT=1.678 min.

¹HNMR: 400 MHz DMSO δ 8.16 (s, 1H), 7.66 (d, J=1.6 Hz, 1H), 7.49 (d, J=8.8 Hz, 1H), 7.27 (dd, J=1.6, 8.5 Hz, 1H), 6.95 (t, J=8.8 Hz, 1H), 6.57 (br d, J=12.0 Hz, 2H), 6.43 (s, 1H), 3.86 (s, 3H), 3.56-3.52 (m, 4H), 2.79-2.74 (m, 1H), 2.12 (s, 3H), 1.76-1.69 (m, 2H), 1.37-1.32 (m, 2H).

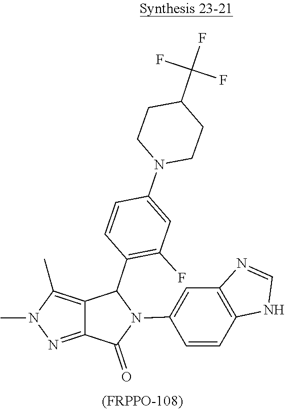 [SMILES: Cc1c2c(nn1C)C(=O)N(c1ccc3[nH]cnc3c1)C2c1ccc(N2CCC(C(F)(F)F)CC2)cc1F]

FRPPO-108 (4.83 mg, 9.21 μmol, 4.72% yield, 97.8% purity) was obtained using an analogous method as a white solid.

LCMS: RT=0.956 min, m/z=513.3 (M+H)⁺.

HPLC: RT=3.027 min.

¹HNMR: 400 MHz DMSO δ 12.22 (s, 1H), 8.18 (s, 1H), 7.68 (d, J=1.8 Hz, 1H), 7.49 (d, J=8 Hz, 1H), 7.30 (d, J=8 Hz, 1H), 6.99 (t, J=8 Hz, 1H), 6.65-6.60 (m, 2H), 6.45 (s, 1H), 3.87 (s, 3H), 3.75-3.70 (m, 2H), 2.64-2.52 (m, 2H), 2.51-2.46 (m, 1H), 2.12 (s, 3H), 1.80 (d, J=11.8 Hz, 2H), 1.44-1.40 (m, 2H).

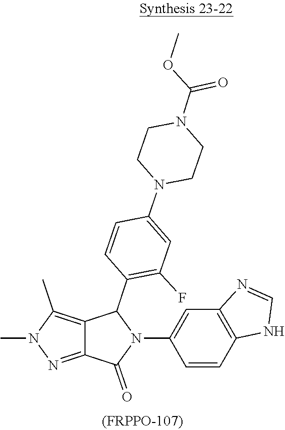 [SMILES: COC(=O)N1CCN(c2ccc(C3c4c(nn(C)c4C)C(=O)N3c3ccc4[nH]cnc4c3)c(F)c2)CC1]

FRPPO-107 (112.8 mg, 202 μmol, 33.8% yield, 90.0% purity) was obtained using an analogous method as a white solid.

LCMS: RT=0.860 min, m/z=504.4 (M+1)⁺.

HPLC: RT=2.295 min.

¹H NMR: 400 MHz DMSO δ 12.42 (br s, 1H), 8.17 (s, 1H), 7.66 (d, J=1.6 Hz, 1H), 7.56-7.26 (m, 2H), 7.02-7.00 (m, 1H), 6.64-6.60 (m, 2H), 6.47-6.45 (m, 1H), 3.86 (s, 3H), 3.59 (s, 3H), 3.41-3.38 (m, 4H), 3.11-3.06 (m, 4H), 2.11 (s, 3H).

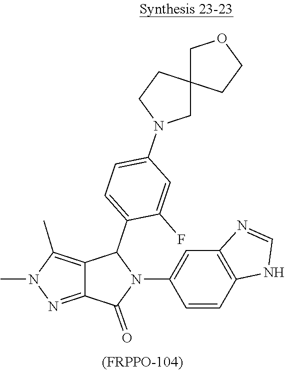 [SMILES: Cc1c2c(nn1C)C(=O)N(c1ccc3[nH]cnc3c1)C2c1ccc(N2CCC3(CCOC3)C2)cc1F]

FRPPO-104 (10.86 mg, 21.3 μmol, 10.4% yield, 95.4% purity) was obtained using an analogous method as a light yellow solid.

LCMS: RT=0.826 min, m/z=487.3 (M+H)⁺.

HPLC: RT=2.495 min.

¹HNMR: 400 MHz DMSO δ 12.45 (s, 1H), 8.19-8.18 (m, 1H), 7.65-7.50 (m, 1H), 7.48 (d, J=8 Hz, 1H), 7.30-7.28 (m, 1H), 6.96-6.91 (m, 1H), 6.63-6.42 (m, 1H), 6.18-6.12 (m, 2H), 3.87 (s, 3H), 3.86-3.73 (m, 2H), 3.51 (s, 2H), 3.24-3.16 (m, 2H), 3.11 (s, 2H), 2.14-2.11 (m, 3H), 1.89-1.81 (m, 4H).

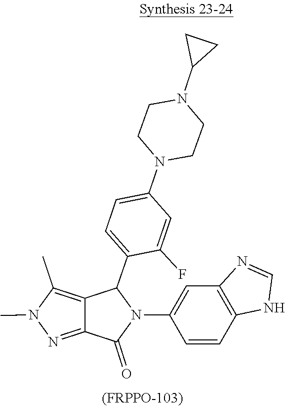 [SMILES: Cc1c2c(nn1C)C(=O)N(c1ccc3[nH]cnc3c1)C2c1ccc(N2CCN(C3CC3)CC2)cc1F]

FRPPO-103 (97.04 mg, 191 μmol, 31.0% yield, 95.8% purity) was obtained using an analogous method as an off-white solid.

LCMS: RT=0.890 min, m/z=486.4 (M+1)⁺.

HPLC: RT=2.593 min.

¹H NMR: 400 MHz DMSO δ 12.46 (s, 1H), 8.16 (d, J=4.4 Hz, 1H), 7.66 (s, 1H), 7.55-7.42 (m, 1H), 7.35-7.26 (m, 1H), 6.98-6.96 (m, 1H), 6.59-6.56 (m, 2H), 6.44 (d, J=10.4 Hz, 1H), 3.85 (s, 3H), 3.02-3.00 (m, 4H), 2.55-2.54 (m, 4H), 2.11 (s, 3H), 1.60-1.55 (m, 1H), 0.41-0.37 (m, 2H), 0.30-0.28 (m, 2H).

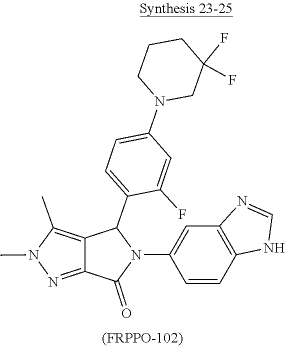 [SMILES: Cc1c2c(nn1C)C(=O)N(c1ccc3[nH]cnc3c1)C2c1ccc(N2CCCC(F)(F)C2)cc1F]

FRPPO-102 (43.08 mg, 83.2 μmol, 40.0% yield, 92.8% purity) was obtained using an analogous method as a light yellow solid.

LCMS: RT=0.914 min, m/z=481.3 (M+1)⁺.

HPLC: RT=2.669 min.

¹HNMR: EW18244-400 MHz DMSO δ 12.43 (br s, 1H), 8.18 (s, 1H), 7.68 (s, 1H), 7.56-7.43 (m, 1H), 7.38-7.22 (m, 1H), 7.01-6.99 (m, 1H), 6.71-6.64 (m, 2H), 6.46 (d, J=8 Hz, 1H), 3.87 (s, 3H), 3.51-3.40 (m, 2H), 3.20-3.18 (m, 2H), 2.12 (s, 3H), 2.02-1.95 (m, 2H), 1.71-1.66 (m, 2H).

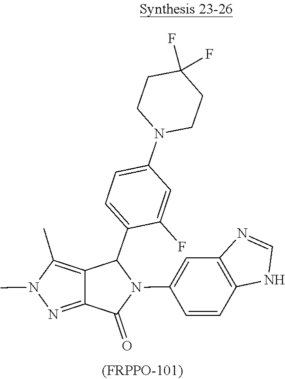 [SMILES: Cc1c2c(nn1C)C(=O)N(c1ccc3[nH]cnc3c1)C2c1ccc(N2CCC(F)(F)CC2)cc1F]

FRPPO-101 (45.98 mg, 95.7 μmol, 20.2% yield, 94.3% purity) was obtained using an analogous method as a yellow solid.

LCMS: RT=0.821 min, m/z=481.3 (M+1)⁺.

HPLC: RT=2.707 min.

¹HNMR: 400 MHz CDCl₃ δ 7.92 (s, 1H), 7.78 (s, 1H), 7.52 (d, J=8.4 Hz, 1H), 7.22 (d, J=8.4 Hz, 1H), 6.89-6.84 (m, 1H), 6.51-6.49 (m, 2H), 6.46 (s, 1H), 3.93 (s, 3H), 3.30-3.27 (m, 4H), 2.19 (s, 3H), 2.05-1.96 (m, 4H).

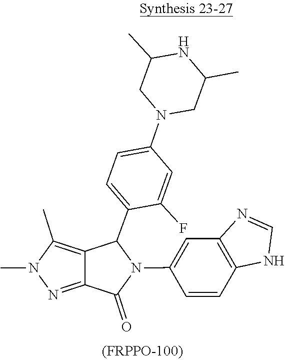 [SMILES: Cc1c2c(nn1C)C(=O)N(c1ccc3[nH]cnc3c1)C2c1ccc(N2CC(C)NC(C)C2)cc1F]

FRPPO-100 (37.4 mg, 77.2 μmol, 36.5% yield, 97.6% purity) was obtained using an analogous method as an off-white solid.

LCMS: RT=0.841 min, m/z=474.4 (M+1)⁺.

HPLC: RT=2.162 min.

¹H NMR: 400 MHz DMSO δ 12.53 (s, 1H), 8.17 (d, J=4.0 Hz, 1H), 7.66 (d, J=1.6 Hz, 1H), 7.56-7.54 (m, 1H), 7.45-7.35 (m, 1H), 0.6.98-0.96 (m, 1H), 6.59-6.56 (m, 2H), 6.44 (d, J=11.2 Hz, 1H), 3.87 (s, 3H), 3.48-3.44 (m, 2H), 2.70-2.68 (m, 2H), 2.12 (s, 3H), 2.08-2.02 (m, 2H), 1.06 (t, J=7.2 Hz, 1H), 0.96 (d, J=6.0 Hz, 6H).

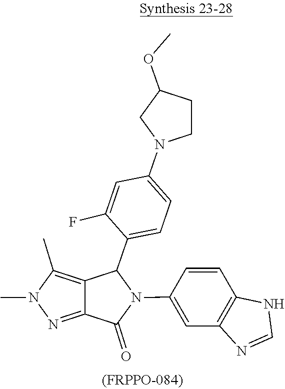 [SMILES: COC1CCN(c2ccc(C3c4c(nn(C)c4C)C(=O)N3c3ccc4[nH]cnc4c3)c(F)c2)C1]

FRPPO-084 (52.4 mg, 110 μmol, 25.4% yield, 96.9% purity) was obtained using an analogous method as a white solid.

LCMS: RT=0.840 min, m/z=461.1 (M+1)⁺.

HPLC: RT=2.493 min.

¹HNMR: 400 MHz DMSO δ 12.43 (s, 1H), 8.18 (d, J=8.4 Hz, 1H), 7.70-7.64 (m, 1H), 7.49-7.46 (m, 1H), 7.30-7.25 (m, 2H), 6.62 (m, 1H), 6.18-6.16 (m, 1H), 6.13 (m, 1H), 3.99-3.88 (m, 1H), 3.87 (d, J=2.4 Hz, 3H), 3.31-3.28 (m, 3H), 3.27 (m, 1H), 3.19 (d, J=0.8 Hz, 2H), 3.15-3.13 (m, 2H), 2.12 (d, J=10.0 Hz, 3H), 1.97-1.95 (m, 1H).

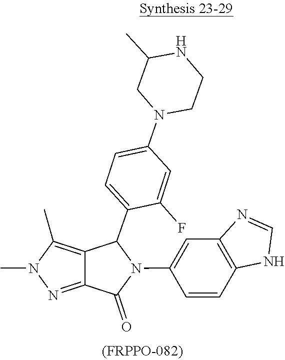 [SMILES: Cc1c2c(nn1C)C(=O)N(c1ccc3[nH]cnc3c1)C2c1ccc(N2CCNC(C)C2)cc1F]

FRPPO-082 (30.35 mg, 66.1 μmol, 15.2% yield, 94.7% purity) was obtained using an analogous method as an off-white solid.

LCMS: RT=0.802 min, m/z=460.1 (M+1)⁺.

HPLC: RT=2.052 min.

¹HNMR: 400 MHz DMSO δ 12.43 (s, 1H), 8.18 (s, 1H), 7.66 (d, J=1.6 Hz, 1H), 7.45 (s, 1H), 7.29 (s, 1H), 6.98-6.94 (m, 1H), 6.58-6.50 (m, 2H), 6.44-6.43 (m, 1H), 3.86 (s, 3H), 3.45-3.40 (m, 2H), 2.85 (d, J=11.6 Hz, 1H), 2.63-2.59 (m, 2H), 2.45-2.44 (m, 1H), 2.11 (s, 3H), 2.09 (m, 1H), 0.95-0.91 (m, 3H).

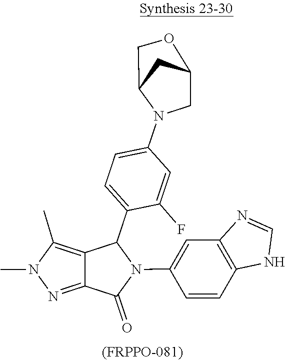 [SMILES: Cc1c2c(nn1C)C(=O)N(c1ccc3[nH]cnc3c1)C2c1ccc(N2C[C@@H]3C[C@H]2CO3)cc1F]

FRPPO-081 (10.34 mg, 22.1 μmol, 5.06% yield, 97.8% purity) was obtained using an analogous method as an off-white solid.

LCMS: RT=0.796 min, m/z=459.1 (M+1)⁺.

HPLC: RT=2.151 min.

¹HNMR: 400 MHz DMSO δ 12.43 (d, J=4.0 Hz, 1H), 8.17 (d, J=4.4 Hz, 1H), 7.66 (s, 1H), 7.56-7.54 (m, 1H), 7.45-7.25 (m, 1H), 6.94-6.93 (m, 1H), 6.42 (br d, J=10.4 Hz, 1H), 6.32-6.24 (m, 2H), 4.55 (s, 1H), 4.45 (br s, 1H), 3.86 (s, 3H), 3.65 (dd, J=1.2, 7.3 Hz, 1H), 3.51 (dd, J=3.2, 7.4 Hz, 1H), 2.89-2.85 (m, 1H), 2.12-2.11 (m, 3H), 1.81-1.75 (m, 2H).

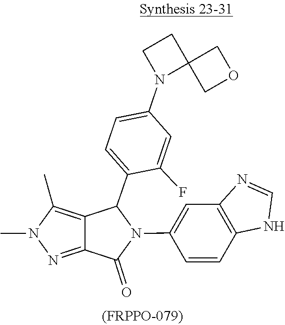 [SMILES: Cc1c2c(nn1C)C(=O)N(c1ccc3[nH]cnc3c1)C2c1ccc(N2CCC23COC3)cc1F]

FRPPO-079 (6.81 mg, 14.4 μmol, 6.60% yield, 96.9% purity) was obtained using an analogous method as a yellow solid.

LCMS: RT=0.785 min, m/z=459.2 (M+H)⁺.

HPLC: RT=2.245 min.

¹HNMR: 400 MHz DMSO δ 12.50 (s, 1H), 8.25 (d, J=4 Hz, 1H), 7.74 (d, J=1.2 Hz, 1H), 7.64-7.50 (m, 1H), 7.43-7.34 (m, 1H), 7.12-7.11 (m, 1H), 6.52 (d, J=10.4 Hz, 1H), 6.43-6.39 (m, 2H), 4.99 (br d, J=8. Hz, 2H), 4.65 (d, J=8 Hz, 2H), 3.93 (s, 3H), 3.60 (br t, J=7.6 Hz, 2H), 2.50-2.46 (m, 2H), 2.19 (s, 3H).

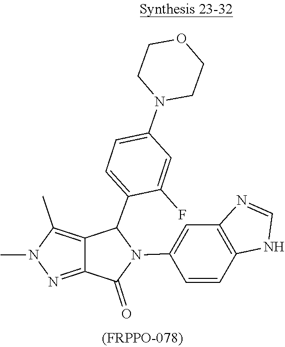 [SMILES: Cc1c2c(nn1C)C(=O)N(c1ccc3[nH]cnc3c1)C2c1ccc(N2CCOCC2)cc1F]

FRPPO-078 (35.47 mg, 77.9 μmol, 17.4% yield, 98.1% purity was obtained using an analogous method as an off-white solid.

LCMS: RT=0.800 min, m/z=447.1 (M+1)⁺.

HPLC: RT=2.185 min.

¹HNMR: 400 MHz DMSO δ 12.42 (s, 1H), 8.18 (s, 1H), 7.67-7.66 (d, J=1.6 Hz, 1H), 7.49 (br d, J=6.4 Hz, 1H), 7.30 (br d, J=7.6 Hz, 1H), 7.01 (t, J=8.8 Hz, 1H), 6.63-6.57 (m, 2H), 6.46 (s, 1H), 3.86 (s, 3H), 3.62 (t, J=4.8 Hz, 4H), 3.06-2.99 (m, 4H), 2.11 (s, 3H).

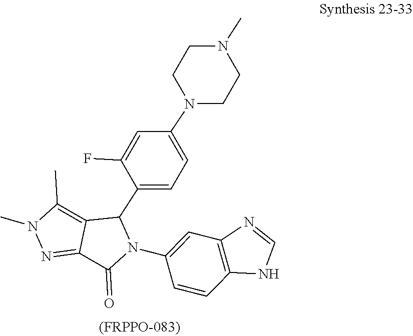 [SMILES: Cc1c2c(nn1C)C(=O)N(c1ccc3[nH]cnc3c1)C2c1ccc(N2CCN(C)CC2)cc1F]

FRPPO-083 (55.81 mg, 116 μmol, 26.7% yield, 95.7% purity) was obtained using an analogous method as a white solid.

LCMS: RT=0.805 min, m/z=460.2 (M+1)⁺.

HPLC: RT=2.120 min.

¹HNMR: 400 MHz DMSO δ 12.42 (d, J=4.0 Hz, 1H), 8.17 (d, J=4.0 Hz, 1H), 7.66 (s, 1H), 7.56-7.42 (m, 1H), 7.35-7.26 (m, 1H), 6.99-6.97 (m, 1H), 6.61-6.57 (m, 2H), 6.45 (d, J=10.4 Hz, 1H), 3.86 (s, 3H), 3.05 (dd, J=3.6, 5.7 Hz, 4H), 2.33 (t, J=4.8 Hz, 4H), 2.15 (s, 3H), 2.11 (s, 3H).

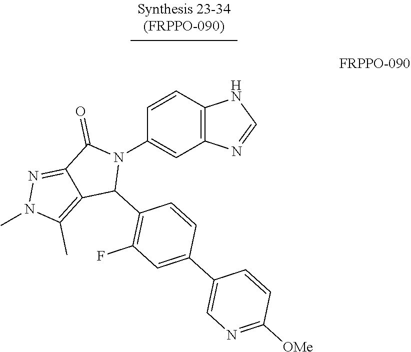 [SMILES: COc1ccc(-c2ccc(C3c4c(nn(C)c4C)C(=O)N3c3ccc4[nH]cnc4c3)c(F)c2)cn1]

Starting from (6-methoxypyridin-3-yl)boronic acid, FRPPO-090 (135.74 mg, 284 μmol, 66.6% yield, 98.1% purity) was obtained using an analogous Suzuki reaction as an off-white solid.

LCMS: RT=0.882 min, m/z=469.3 (M+1)⁺.

HPLC: RT=2.328 min.

¹HNMR: 400 MHz DMSO δ 12.43 (s, 1H), 8.44 (d, J=2.4 Hz, 1H), 8.17 (s, 1H), 7.96 (dd, J=2.4, 8.7 Hz, 1H), 7.76 (s, 1H), 7.50-7.47 (m, 2H), 7.39-7.38 (m, 1H), 7.33-7.32 (m, 2H), 6.84 (d, J=8.8 Hz, 1H), 6.67 (br s, 1H), 3.86 (d, J=8.0 Hz, 6H), 2.15 (s, 3H).

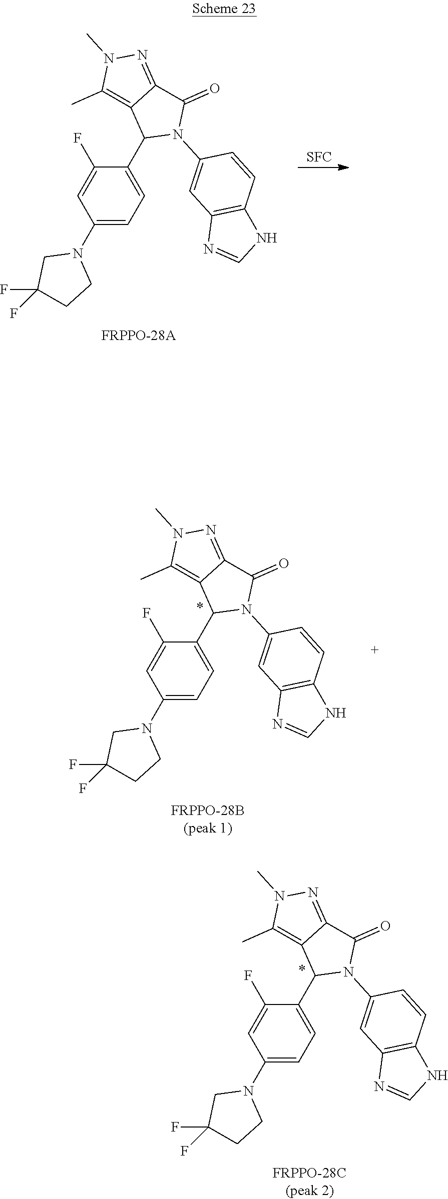

### Synthesis 24-1

FRPPO-28A was purified by multiple injections on chiral SFC (column DAICEL CHIRALPAK AD 250×30 mm, 10 μm; mobile phase A—supercritical CO₂, B—IPA (0.1% NH₄OH); isocratic 60% B, 7 min).

FRPPO-028B (peak 1 RT=2.25 min, 99% purity) was obtained and confirmed by ¹H NMR, LCMS, and SFC.

LCMS: RT=0.90 min, m/z=467 (M+1)⁺.

Chiral SFC: RT=1.50 min.

¹H NMR: 400 MHz DMSO: δ 12.42 (br s, 1H) 8.18 (s, 1H), 7.64 (s, 1H), 7.49 (br dd, 1H, J=8.8, 44), 7.30 (br dd, 1H, J=8.8, 45), 7.00 (m, 1H), 6.45 (br d, 1H, J=10.8), 6.25-6.30 (m, 2H), 3.87 (s, 3H), 3.56-3.63 (m, 2H), 3.39 (m, 2H), 2.44 (m, 2H), 2.11 (s, 3H).

FRPPO-028C (peak 2) (RT=3.22 min, 100% purity) was obtained and confirmed by ¹H NMR, LCMS, and SFC.

LCMS: RT=0.90 min, m/z=467 (M+1)⁺.

Chiral SFC: RT=1.85 min.

¹H NMR: 400 MHz DMSO: δ 12.43 (br s, 1H) 8.17 (s, 1H), 7.64 (s, 1H), 7.48 (br s, 1H), 7.29 (br s, 1H), 7.00 (t, 1H, J=8.8), 6.45 (s, 1H), 6.24-6.30 (m, 2H), 3.86 (s, 3H), 3.55-3.62 (m, 2H), 3.36 (m, 2H), 2.42 (m, 2H), 2.11 (s, 3H).

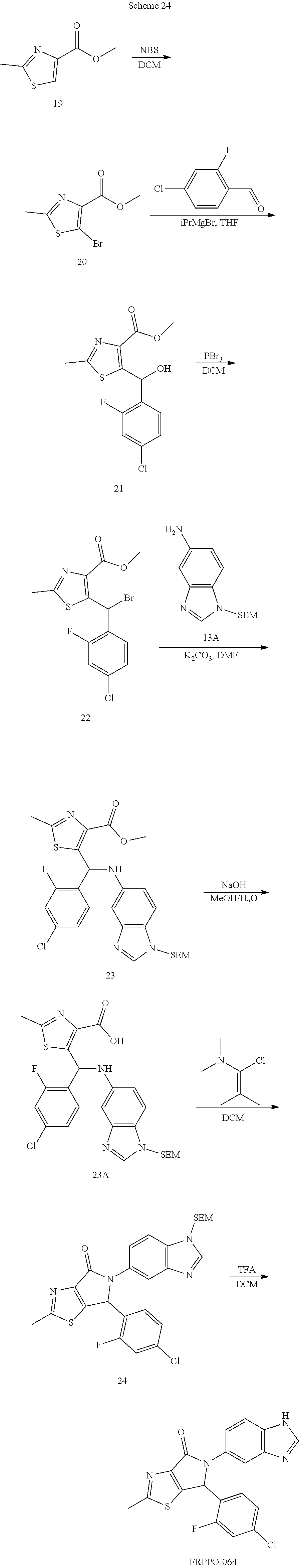

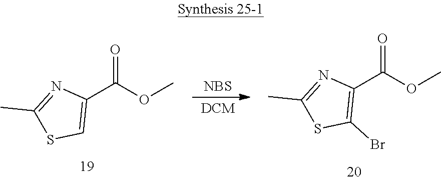

To a solution of 19 (10.0 g, 63.6 mmol, 1.00 eq) in ACN (50 mL) was added NBS (22.6 g, 127 mmol, 2.00 eq). The mixture was stirred at 90° C. for 12 hrs. The reaction mixture was concentrated to give a residue. The residue was diluted with water 100 mL and extracted with DCM 100 mL (50 mL×2). The combined organic layers were washed with brine 100 mL, dried over Na₂SO₄, filtered and concentrated under reduced pressure to give a residue. The residue was purified by flash silica gel chromatography (ISCO; 80 g SepaFlash Silica Flash Column, Eluent of 0-100% ethyl acetate/petroleum ether gradient @ 60 mL/min) to give compound 20 (4.00 g, 16.9 mmol, 26.6% yield) as a yellow solid.

¹H NMR: 400 MHz CDCl₃ δ 3.96 (s, 3H), 2.71 (s, 3H).

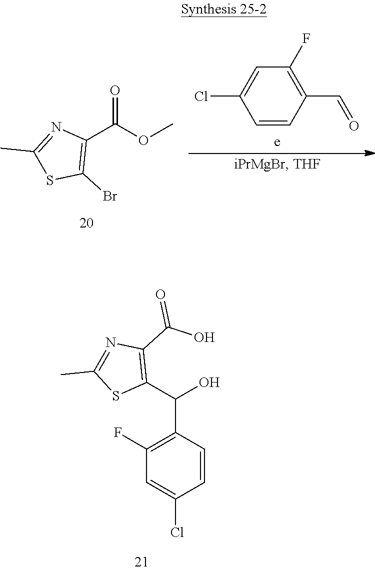

To a solution of compound 20 (3.00 g, 12.7 mmol, 1.00 eq) in THF (20 mL) was added i-PrMgBr (1.00 M, 19.2 mL, 1.51 eq). The mixture was stirred at −70° C. for 0.5 hr, then compound e (3.00 g, 18.9 mmol, 1.49 eq) was added, the mixture was stirred at −40° C. for 1 hr. The reaction mixture was quenched by addition NH₄Cl 30 mL at −40° C., and then diluted with water 100 mL and extracted with ethyl acetate 150 mL (50 mL×3). The combined organic layers were washed with brine 100 mL, dried over Na₂SO₄, filtered and concentrated under reduced pressure to give a residue. The residue was purified by flash silica gel chromatography (ISCO; 40 g SepaFlash Silica Flash Column, Eluent of 0˜50% ethyl acetate/petroleum ether gradient, 60 mL/min) to give compound 21 (1.50 g, 4.75 mmol, 37.3% yield) as a yellow solid.

LCMS: RT=0.907 min, m/z=298.1 (M+1)⁺.

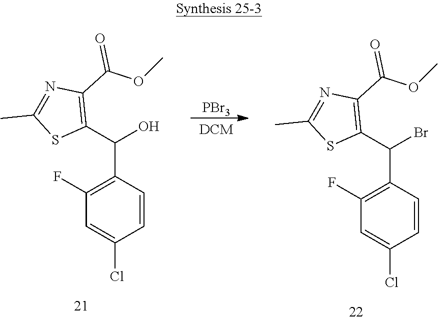

To a solution of compound 21 (1.40 g, 4.43 mmol, 1.00 eq) in DCM (10 mL) was added PBr₃ (7.00 g, 25.8 mmol, 5.83 eq). The mixture was stirred at 25° C. for 2 hrs. The reaction mixture was poured into water, and extracted with DCM 60 mL (20 mL×3). The combined organic layers were washed with brine 20 mL, dried over Na₂SO₄, filtered and concentrated under reduced pressure to give compound 22 (1.70 g, crude) as yellow oil and used in the next step directly without purification.

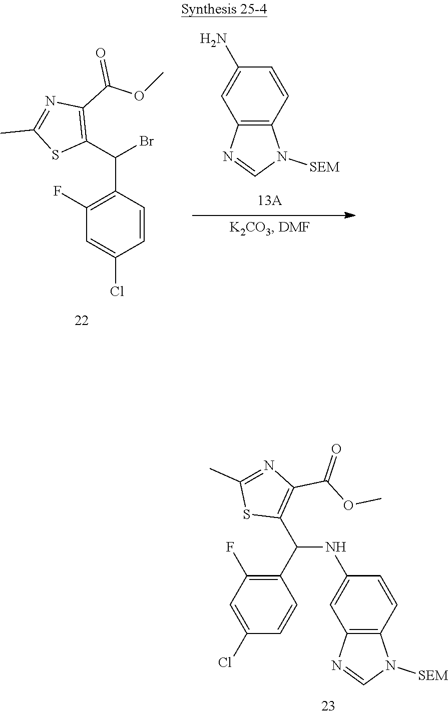

To a solution of compound 22 (1.70 g, 4.47 mmol, 1.00 eq) in DMF (10 mL) was added K₂CO₃ (1.42 g, 10.2 mmol, 2.30 eq) and compound 13A (1.40 g, 5.31 mmol, 1.19 eq). The mixture was stirred at 90° C. for 1 hr. The reaction mixture was partitioned between water 100 mL and ethyl acetate 120 mL. The organic phase was separated, washed with water 120 mL (40 mL×3) and brine 100 mL, dried over Na₂SO₄, filtered and concentrated under reduced pressure to give a residue. The crude product was purified by reversed-phase HPLC (0.1% formic acid H₂O/MeCN) to give compound 23 (2.00 g, 3.49 mmol, 78.1% yield, purity: 98.2%) as a yellow oil.

LCMS: RT=1.140 min, m/z=559.2 (M+1)⁺.

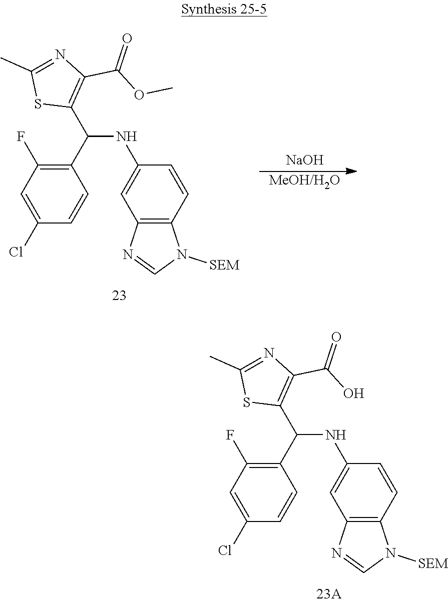 [SMILES: COC(=O)c1nc(C)sc1C(Nc1ccc2c(c1)ncn2COCC[Si](C)(C)C)c1ccc(Cl)cc1F ; "NaOH" ; "MeOH/H2O" ; Cc1nc(C(=O)O)c(C(Nc2ccc3c(c2)ncn3COCC[Si](C)(C)C)c2ccc(Cl)cc2F)s1]

To a solution of compound 23 (1.50 g, 2.67 mmol, 1.00 eq) in MeOH (18 mL) was added NaOH (750 mg, 18.7 mmol, 7.01 eq) in H₂O (6 mL). The mixture was stirred at 80° C. for 1 hr. The reaction mixture was concentrated under reduced pressure to remove MeOH. The residue was adjusted to pH 6 with aq HCl (1M) and extracted with ethyl acetate 150 mL (50 mL×3). The combined organic layers were washed with brine 60 mL, dried over Na₂SO₄, filtered and concentrated under reduced pressure to give compound 23A (1.20 g, crude) as yellow oil, which was used in the next step directly without any purification.

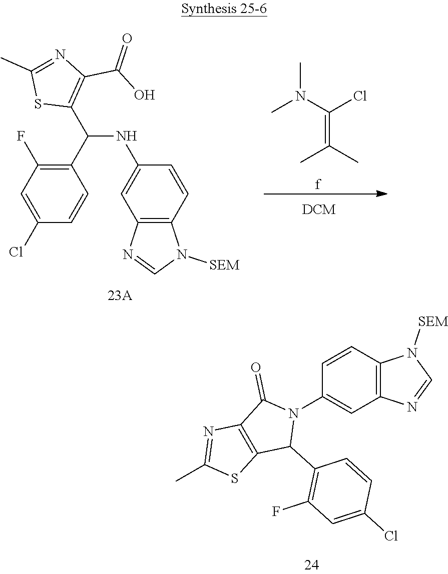

To a solution of compound 23A (1.00 g, 1.83 mmol, 1.00 eq) in DCM (10 mL) was added compound f (505 mg, 3.78 mmol, 500 uL, 2.07 eq) at 0° C. The mixture was stirred at 0° C. for 0.5 hrs. The reaction mixture was partitioned between water 50 mL and DCM 50 mL. The organic phase was separated, washed with brine 100 mL (50 mL×2), dried over Na₂SO₄, filtered and concentrated under reduced pressure to give a residue. The residue was purified by reversed-phase HPLC (0.1% NH₄OH H₂O/MeCN) to give compound 24 (300 mg, 542 μmol, 29.6% yield, 95.7% purity) as a yellow oil, which was used directly in the subsequent reaction.

LCMS: RT=1.090 min, m/z=529.2 (M+1)⁺.

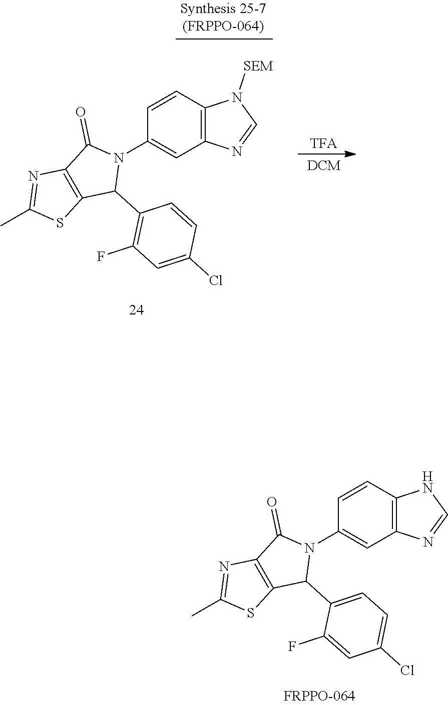

To a solution of compound 24 (100 mg, 189 μmol, 1.00 eq) in DCM (1.5 mL) was added TFA (770 mg, 6.75 mmol, 0.5 mL, 35.7 eq). The mixture was stirred at 25° C. for 1 hr. The reaction mixture was adjusted to pH 8 with NH₃H₂O. The mixture was diluted with water 20 mL and extracted with DCM 30 mL (10 mL×3). The combined organic layers were washed with brine 20 mL, dried over Na₂SO₄, filtered and concentrated under reduced pressure to give a residue. The residue was purified by prep-HPLC (column: Waters Xbridge 150×25 mm, 5 μm; mobile phase: (0.05% NH₄OH) A—water, B—ACN; gradient 22%-52% B, 10 min) to give FRPPO-064 (48 mg, 109 μmol, 58.1% yield, 91.2% purity) as a yellow solid.

LCMS: RT=1.720 min. m/z=399.0 (M+1)⁺.

¹H NMR: 400 MHz CDCl₃ δ 8.15-7.99 (m, 1H), 7.99-7.84 (m, 1H), 7.55-7.53 (m, 1H), 7.22-7.10 (m, 1H), 7.08-7.06 (m, 1H), 7.01-6.98 (m, 2H), 6.96-6.57 (m, 1H), 2.86 (s, 3H).

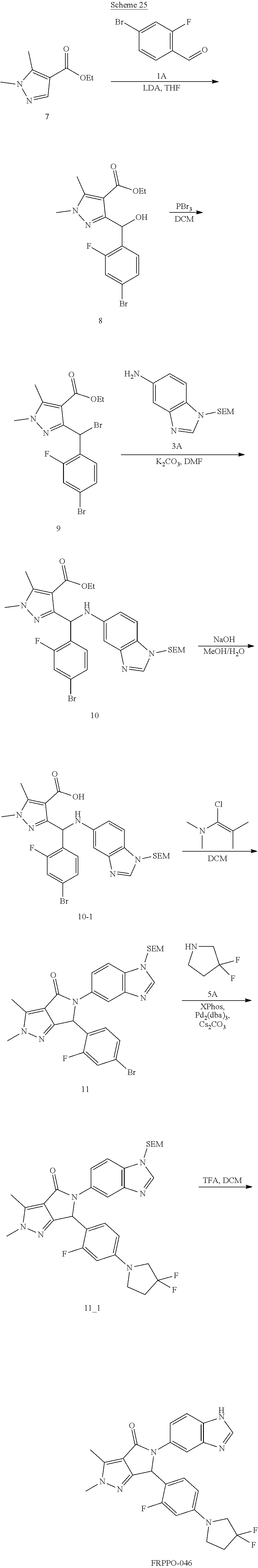

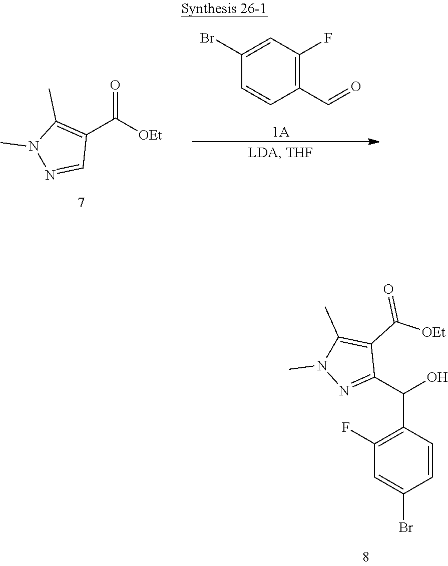

To a solution of compound 7 (2.00 g, 11.8 mmol, 1.00 eq) in THF (20 mL) was added LDA (2.00 M, 11.8 mL, 2.00 eq) at −78° C., the mixture was stirred at −78° C. for 1 hr. Then compound 1A (4.83 g, 23.7 mmol, 2.00 eq) was added and the mixture was stirred at −78° C. for 1 hr. The reaction mixture was quenched by addition saturated NH₄Cl 5 mL at −78° C., and then diluted with EtOAc 50 mL and dried over Na₂SO₄, filtered and concentrated under reduced pressure to give a residue. The crude product was purified by reversed-phase HPLC (0.1% NH₄OH H₂O/MeCN) to give Compound 8 (3.20 g, 8.62 mmol, 72.5% yield) as a yellow oil.

¹H NMR: 400 MHz CDCl₃ δ 7.44 (t, J=8.3 Hz, 1H), 7.30-7.28 (m, 1H), 7.20 (dd, J=1.9, 10.1 Hz, 1H), 6.54-6.46 (m, 1H), 6.13 (s, 1H), 4.35-4.27 (m, 2H), 3.85 (s, 3H), 2.40 (s, 3H), 1.36 (t, J=7.2 Hz, 3H).

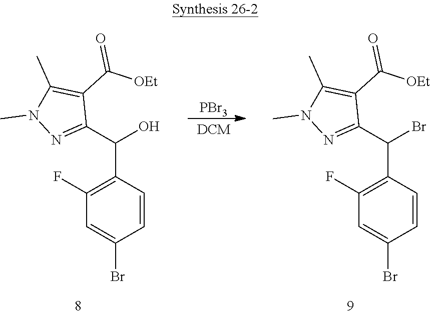

To a solution of compound 8 (1.00 g, 2.69 mmol, 1.00 eq) in DCM (5 mL) was added PBr₃ (3.65 g, 13.4 mmol, 5.00 eq). The mixture was stirred at 25° C. for 12 hrs. TLC (petroleum ether:ethyl acetate=3:1) indicated compound 9 (R_{f}=0.30) was consumed completely and new spots (R_{f}=0.40, 0.50) were formed. The mixture was poured into the water (20 mL) and extracted with DCM (10 mL×2). The combined organic layers were washed with Na₂CO₃ (10 mL), dried over Na₂SO₄, filtered and concentrated under reduced pressure to give compound 9 (400 mg, 921.4 μmol, 34.2% yield) as a light yellow oil which was used immediately in the next step.

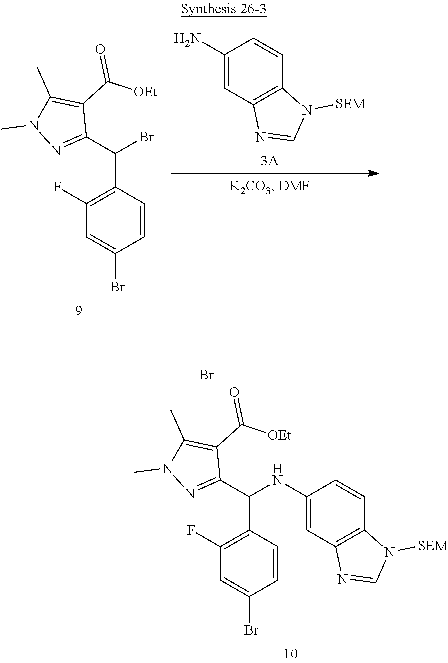 [SMILES: CCOC(=O)c1c(C(Br)c2ccc(Br)cc2F)nn(C)c1C ; C[Si](C)(C)CCOCn1cnc2cc(N)ccc21 ; Br ; CCOC(=O)c1c(C(Nc2ccc3c(c2)ncn3COCC[Si](C)(C)C)c2ccc(Br)cc2F)nn(C)c1C]

To a solution of compound 9 (400 mg, 921 μmol, 1.00 eq) and compound 3A (243 mg, 922 μmol, 1.00 eq) in DMF (5 mL) was added K₂CO₃ (255 mg, 1.85 mmol, 2.00 eq). The mixture was stirred at 80° C. for 4 hrs. The mixture was poured into the water (20 mL) and extracted with EtOAc (10 mL×2). The combined organic layers were washed with brine (10 mL×2), dried over Na₂SO₄, filtered and concentrated under reduced pressure to give a residue. The crude product was purified by reversed-phase HPLC (0.1% formic acid H₂O/MeCN) to give compound 10 (300 mg, 486 μmol, 52.8% yield) as a yellow oil.

¹H NMR: MHz CDCl₃ δ 7.88 (s, 1H), 7.36-7.34 (m, 1H), 7.24-7.20 (m, 2H), 7.01-7.00 (m, 1H), 6.84-6.72 (m, 2H), 6.27 (s, 1H), 5.46-5.41 (m, 2H), 4.28-4.25 (m, 2H), 3.95-3.94 (m, 3H), 3.51-3.45 (m, 2H), 2.37-2.36 (m, 3H), 1.35-1.30 (m, 3H), 0.92-0.84 (m, 2H), 0.05-0.04 (m, 9H).

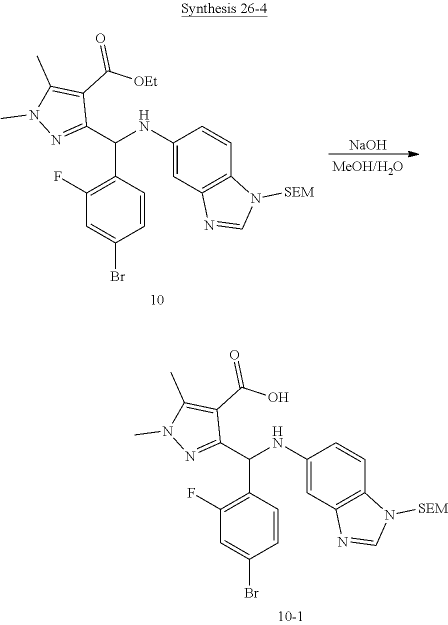 [SMILES: CCOC(=O)c1c(C(Nc2ccc3c(c2)ncn3COCC[Si](C)(C)C)c2ccc(Br)cc2F)nn(C)c1C ; "NaOH" ; Cc1c(C(=O)O)c(C(Nc2ccc3c(c2)ncn3COCC[Si](C)(C)C)c2ccc(Br)cc2F)nn1C]

To a solution of compound 10 (270 mg, 437 μmol, 1.00 eq) in MeOH (5 mL) and H₂O (1 mL) was added NaOH (88.0 mg, 2.20 mmol, 5.02 eq). The mixture was stirred at 70° C. for 6 hrs. The mixture was concentrated under reduced pressure to give a residue. The residue was diluted in water (20 mL) and adjusted to pH 3 with HCl solution (1 M). The mixture was extracted with EtOAc (10 mL×2). The combined organic layers were dried over anhydrous sodium sulfate, filtrated and concentrated to give compound 10-1 (300 mg, crude) as a yellow solid, which was used in the next step directly.

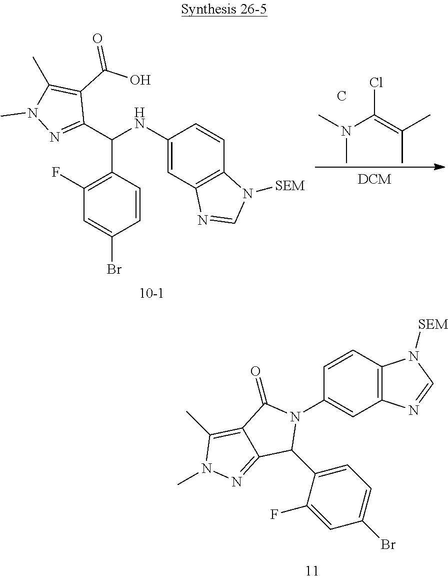

To a solution of compound 10-1 (300 mg, 509 μmol, 1.00 eq) in DCM (5 mL) was added Ghosez's reagent C (186 mg, 1.39 mmol, 184 uL, 2.73 eq) at 0° C. The mixture was stirred at 0° C. for 2 hrs. The mixture was concentrated under reduced pressure to give a residue. The crude product was purified by reversed-phase HPLC (0.1% NH₄OH H₂O/MeCN) to give compound 11 (200 mg, 350 μmol, purity: 97.7%, 68.8% yield) as a yellow gum, which was confirmed by LCMS.

LCMS: RT=1.106 min, m/z=570.3 (M+1)⁺.

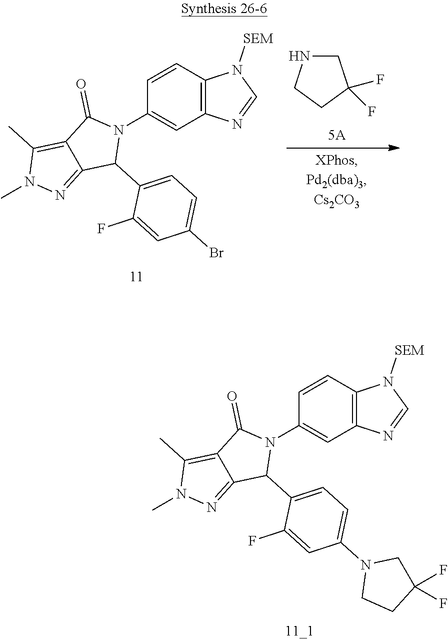

To a solution of compound 11 (140 mg, 245 μmol, 1.00 eq) and compound 5a (56.0 mg, 390 μmol, 1.59 eq, HCl) in dioxane (5 mL) was added XPhos (12.0 mg, 25.1 μmol, 0.001 eq), Pd₂(dba)₃ (22.0 mg, 24.0 μmol, 0.01 eq) and Cs₂CO₃ (160 mg, 491 μmol, 2.00 eq).

The mixture was stirred at 100° C. for 12 hrs. The mixture was filtered and concentrated under reduced pressure to give a residue. The residue was purified by prep-TLC (SiO₂, Dichloromethane/Methanol=10/1; TLC; Dichloromethane/Methanol=10/1, R_{f}=0.30) to give Compound 11_1 (180 mg, crude) as a yellow oil, which was confirmed by LCMS and used in the next step directly.

LCMS: RT=1.065 min. m/z=597.4 (M+1)⁺.

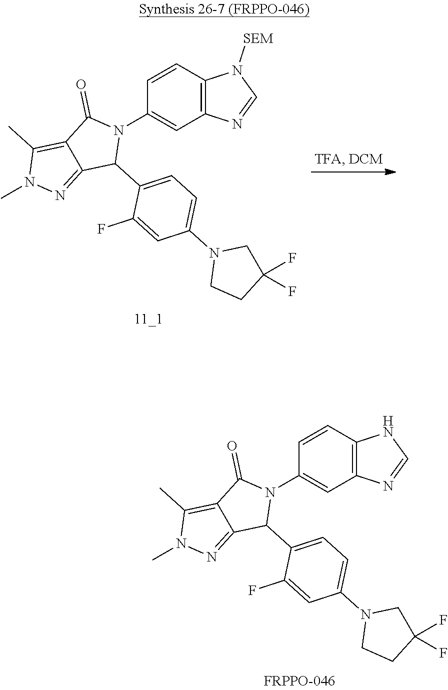 [SMILES: Cc1c2c(nn1C)C(c1ccc(N3CCC(F)(F)C3)cc1F)N(c1ccc3c(c1)ncn3COCC[Si](C)(C)C)C2=O ; Cc1c2c(nn1C)C(c1ccc(N3CCC(F)(F)C3)cc1F)N(c1ccc3[nH]cnc3c1)C2=O]

To a solution of compound 11_1 (90.0 mg, 150 μmol, 1.00 eq) in DCM (3 mL) was added TFA (3.08 g, 27.0 mmol, 2 mL, 179 eq). The mixture was stirred at 25° C. for 10 hrs. The reaction mixture was adjusted to pH 8 with Na₂CO₃ (aq), then extracted with DCM (20 mL×2). The combined organic layers were dried over Na₂SO₄, filtered and concentrated under reduced pressure to give a residue. The residue was purified by prep-HPLC (column Waters Xbridge 150×25 mm, 5 μm; mobile phase: (0.05% NH₄OH) A—water, B—ACN; gradient 27%-47% B, 10 min) to give FRPPO-046 (7.14 mg, 14.8 μmol, 9.84% yield, 97.0% purity) as an off-white solid.

LCMS: RT=0.886 min, m/z=467.3 (M+1)⁺.

HPLC: RT=1.977 min.

¹H NMR: 400 MHz methanol-d4 δ 8.14 (s, 1H), 7.56-7.55 (m, 2H), 7.26-7.24 (m, 1H), 7.00-6.96 (m, 1H), 6.47 (s, 1H), 6.34-6.34 (m, 2H), 3.61-3.54 (m, 5H), 3.45-3.31 (m, 2H), 2.48-2.41 (m, 5H).

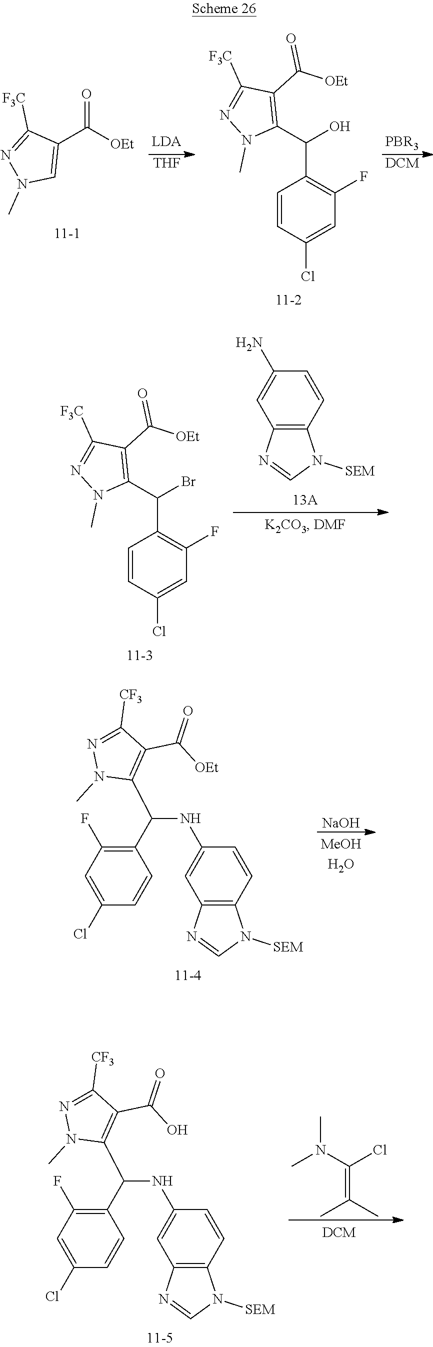

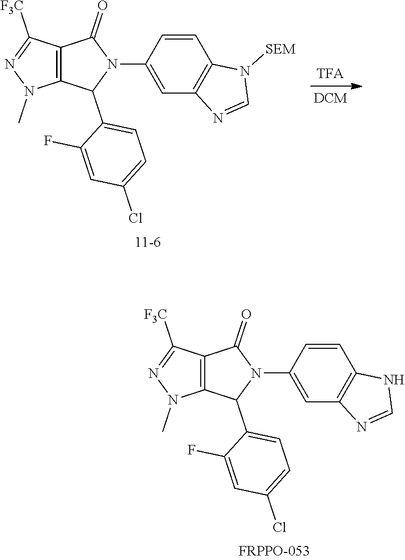 [SMILES: Cn1nc(C(F)(F)F)c2c1C(c1ccc(Cl)cc1F)N(c1ccc3c(c1)ncn3COCC[Si](C)(C)C)C2=O ; Cn1nc(C(F)(F)F)c2c1C(c1ccc(Cl)cc1F)N(c1ccc3[nH]cnc3c1)C2=O]

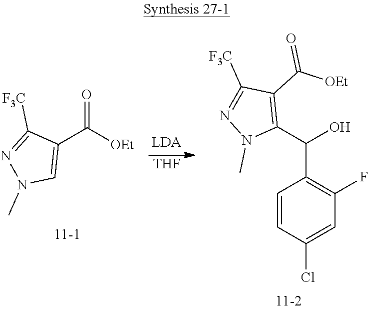 [SMILES: CCOC(=O)c1cn(C)nc1C(F)(F)F ; CCOC(=O)c1c(C(F)(F)F)nn(C)c1C(O)c1ccc(Cl)cc1F ; "THF"]

To a solution of compound 11-1 (2.00 g, 9.00 mmol, 1.00 eq) in THF (50 mL) was added LDA (2 M, 9.00 mL, 2.00 eq) at −78° C. The mixture was stirred at −78° C. for 0.5 hr, then compound A (2.80 g, 17.7 mmol, 1.96 eq) was added to the mixture and the mixture was stirred at −78° C. for 0.5 hr. The mixture was quenched with water (200 mL) and extracted with EtOAc (150 mL×2). The combined organic layers were dried over anhydrous sodium sulfate, filtrated and concentrated to give a residue. The residue was purified by reversed-phase HPLC (0.1% formic acid H₂O/MeCN) to give compound 11-2 (2.00 g, crude) as a yellow solid, which was confirmed by LCMS and used in the next step directly.

LCMS: RT=0.987 min, m/z=380.9 (M+1)⁺.

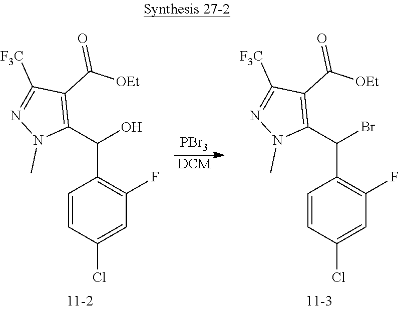

To a solution of compound 11-2 (2.00 g, 5.25 mmol, 1.00 eq) in DCM (30 mL) was added PBr₃ (7.11 g, 26.3 mmol, 5.00 eq). The mixture was stirred at 25° C. for 2 hrs. The mixture was diluted with DCM (30 mL) and washed with water (20 mL), brine (20 mL), dried over anhydrous sodium sulfate, filtered and concentrated to give compound 11-3 (2.00 g, crude) as a yellow oil, which was used in the next step directly.

LCMS: RT=1.118 min, m/z=444.8 (M+1)⁺.

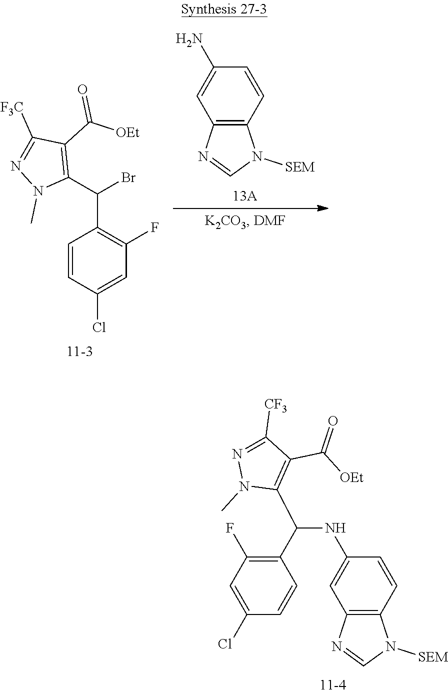 [SMILES: CCOC(=O)c1c(C(F)(F)F)nn(C)c1C(Br)c1ccc(Cl)cc1F ; C[Si](C)(C)CCOCn1cnc2cc(N)ccc21 ; CCOC(=O)c1c(C(F)(F)F)nn(C)c1C(Nc1ccc2c(c1)ncn2COCC[Si](C)(C)C)c1ccc(Cl)cc1F]

To a solution of compound 11-3 (2.00 g, 4.51 mmol, 1.00 eq) and compound 13A (1.19 g, 4.51 mmol, 1.00 eq) in DMF (20 mL) was added K₂CO₃ (2.00 g, 14.5 mmol, 3.21 eq). The mixture was stirred at 80° C. for 1 hr. The mixture was poured into water (200 mL) and extracted with EtOAc (200 mL×2). The combined organic layers were dried over anhydrous sodium sulfate, filtrated and concentrated to give a residue. The residue was purified by reversed-phase HPLC (0.1% formic acid H₂O/MeCN) to give compound 11-4 (1.80 g, crude) as a yellow solid, which was confirmed by LCMS and used in the next step directly.

LCMS: RT=0.823 min. m/z=626.0 (M+1)⁺.

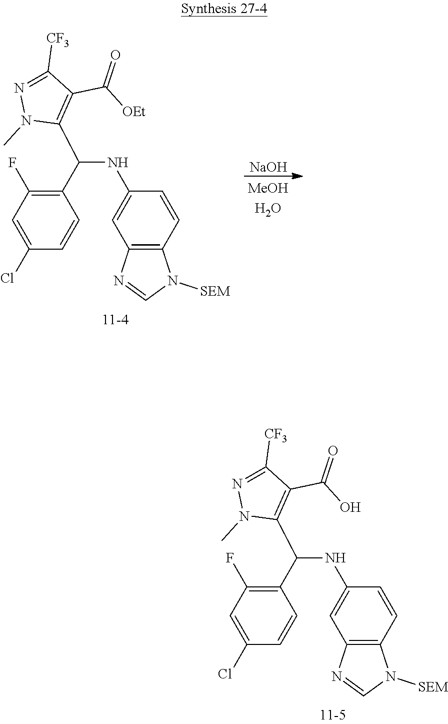 [SMILES: CCOC(=O)c1c(C(F)(F)F)nn(C)c1C(Nc1ccc2c(c1)ncn2COCC[Si](C)(C)C)c1ccc(Cl)cc1F ; "NaOH" ; Cn1nc(C(F)(F)F)c(C(=O)O)c1C(Nc1ccc2c(c1)ncn2COCC[Si](C)(C)C)c1ccc(Cl)cc1F]

To a solution of compound 11-4 (1.80 g, 2.87 mmol, 1.00 eq) in MeOH (20 mL) and H₂O (7 mL) was added NaOH (2 M, 7.25 mL, 5.04 eq). The mixture was stirred at 25° C. for 12 hrs. The mixture was concentrated under reduced pressure to give a residue. The residue was diluted in water (200 mL) and adjusted to pH 5 with HCl solution (1 M). The mixture was extracted with EtOAc (100 mL×2). The combined organic layers were dried over anhydrous sodium sulfate, filtrated and concentrated to give compound 11-5 (700 mg, crude) as yellow oil, which was used in the next step without any purification.

LCMS: RT=0.906 min, m/z=598.1 (M+1)⁺.

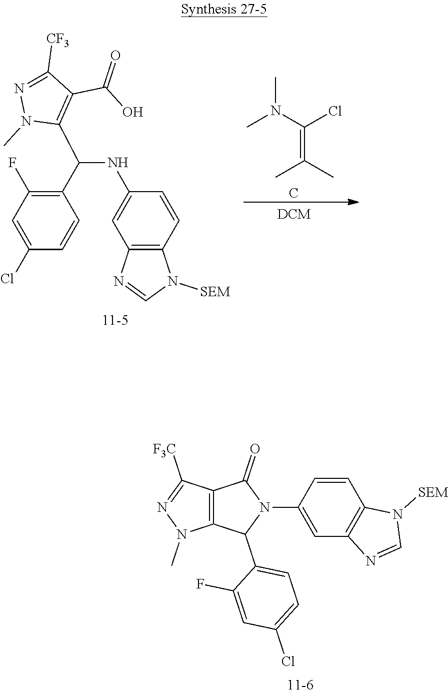

To a solution of compound 11-5 (700 mg, 1.17 mmol, 1.00 eq) in DCM (20 mL) was added compound C (500 mg, 3.74 mmol, 495 uL, 3.20 eq) at 0° C. The mixture was stirred at 0° C. for 2 hrs. The mixture was concentrated under reduced pressure to give a residue. The crude product was purified by reversed-phase HPLC (0.1% formic acid H₂O/MeCN) to give compound 11-6 (320 mg, crude) as a yellow solid, which was confirmed by LCMS(EW17696-103-P1A1) and used in the next step directly.

LCMS: RT=0.941 min. m/z=580.1 (M+1)⁺.

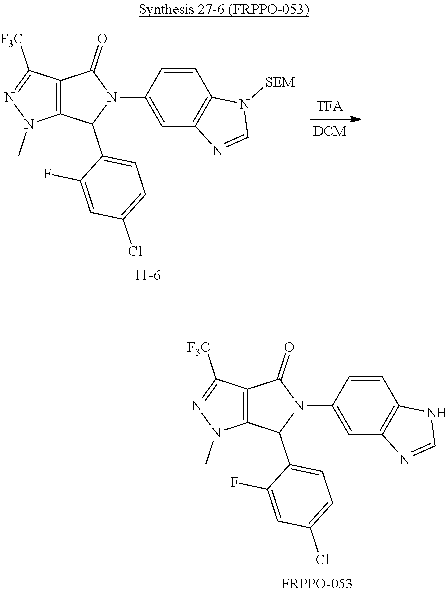 [SMILES: Cn1nc(C(F)(F)F)c2c1C(c1ccc(Cl)cc1F)N(c1ccc3c(c1)ncn3COCC[Si](C)(C)C)C2=O ; Cn1nc(C(F)(F)F)c2c1C(c1ccc(Cl)cc1F)N(c1ccc3[nH]cnc3c1)C2=O]

To a solution of compound 11-6 (40.0 mg, 68.9 μmol, 1.00 eq) in DCM (5 mL) was added TFA (1.54 g, 13.5 mmol, 1 mL). The mixture was stirred at 25° C. for 1 hr. LCMS showed desired mass (RT=0.898 min, m/z=450.0) was detected. The reaction mixture was adjusted to pH 8 with Na₂CO₃ (aq), then extracted with DCM (20 mL×2). The combined organic layers were dried over Na₂SO₄, filtered and concentrated under reduced pressure to give a residue. The residue was purified by prep-HPLC (column: Waters Xbridge 150×25 mm, 5 μm; mobile phase: (0.05% NH₄OH) A—water, B—ACN; gradient 28%-58% B, 10 min) to give FRPPO-053 (8.18 mg, 17.9 μmol, 25.9% yield, 98.3% purity) as a white solid.

LCMS: RT=0.933 min, m/z=450.2 (M+1)⁺.

HPLC: RT=2.110 min.

¹H NMR: 400 MHz CDCl₃ δ 8.01-7.97 (m, 1H), 7.70-7.53 (m, 1H), 7.52-7.51 (m, 1H), 7.19-7.07 (m, 4H), 6.47 (s, 1H), 3.74 (s, 3H).

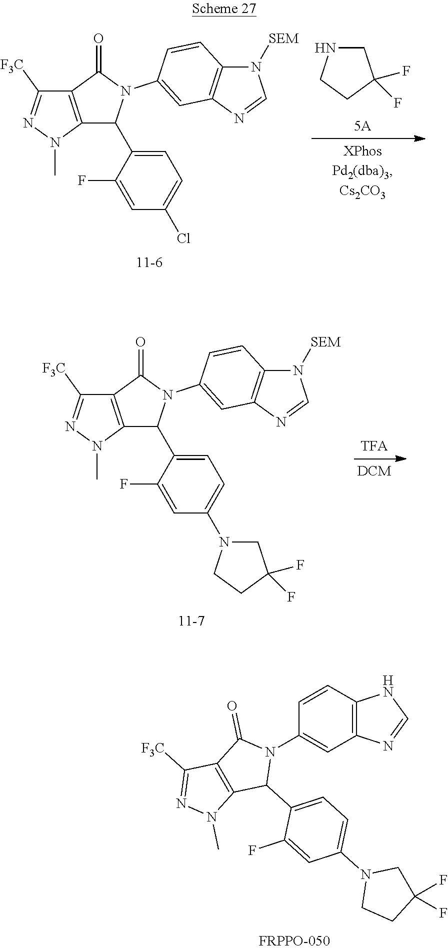 [SMILES: Cn1nc(C(F)(F)F)c2c1C(c1ccc(Cl)cc1F)N(c1ccc3c(c1)ncn3COCC[Si](C)(C)C)C2=O ; FC1(F)CCNC1 ; Cn1nc(C(F)(F)F)c2c1C(c1ccc(N3CCC(F)(F)C3)cc1F)N(c1ccc3c(c1)ncn3COCC[Si](C)(C)C)C2=O ; Cn1nc(C(F)(F)F)c2c1C(c1ccc(N3CCC(F)(F)C3)cc1F)N(c1ccc3[nH]cnc3c1)C2=O]

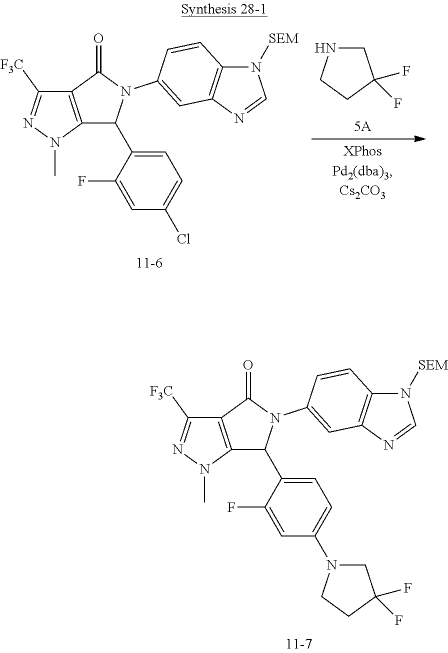 [SMILES: Cn1nc(C(F)(F)F)c2c1C(c1ccc(Cl)cc1F)N(c1ccc3c(c1)ncn3COCC[Si](C)(C)C)C2=O ; FC1(F)CCNC1 ; Cn1nc(C(F)(F)F)c2c1C(c1ccc(N3CCC(F)(F)C3)cc1F)N(c1ccc3c(c1)ncn3COCC[Si](C)(C)C)C2=O]

To a mixture of compound 11-6 (100 mg, 172 μmol, 164 uL, 1.00 eq) and compound 5A

(37.1 mg, 259 μmol, 1.50 eq, HCl) in dioxane (2 mL) was added Cs₂CO₃ (169 mg, 517 μmol, 3.00 eq), Pd₂ (dba)₃ (15.8 mg, 17.2 μmol, 0.1 eq) and XPhos (16.4 mg, 34.5 μmol, 0.2 eq) in one portion under N₂. The mixture was stirred at 90° C. for 3 hours under N₂. The reaction mixture was filtered and concentrated under reduced pressure to give a residue, which was purified by reversed-phase HPLC (0.1% formic acid H₂O/MeCN) to give compound 11-7 (120 mg, crude) as yellow oil and used into the next step without further purification.

LCMS: RT=2.335 min, m/z=651.1 (M+1)⁺.

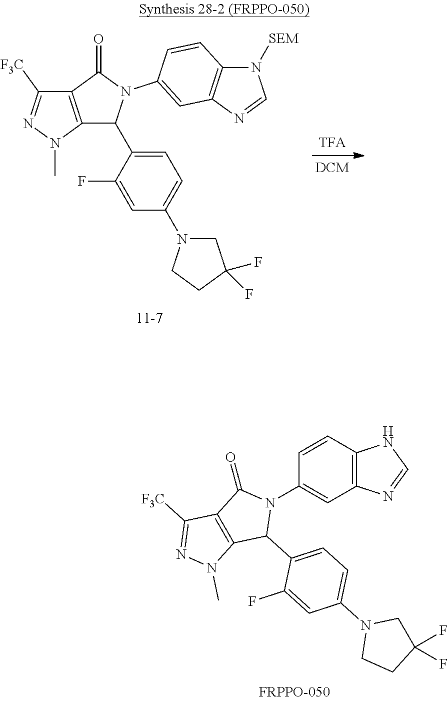 [SMILES: Cn1nc(C(F)(F)F)c2c1C(c1ccc(N3CCC(F)(F)C3)cc1F)N(c1ccc3c(c1)ncn3COCC[Si](C)(C)C)C2=O ; Cn1nc(C(F)(F)F)c2c1C(c1ccc(N3CCC(F)(F)C3)cc1F)N(c1ccc3[nH]cnc3c1)C2=O]

To a solution of compound 11-7 (100 mg, 154 μmol, 1.00 eq) in DCM (10 mL) was added TFA (3.08 g, 27.0 mmol, 2 mL). The mixture was stirred at 25° C. for 12 hrs. The reaction mixture was adjusted to pH 8 with Na₂CO₃ (aq), then extracted with DCM (20 mL×2).

The combined organic layers were dried over Na₂SO₄, filtered and concentrated under reduced pressure to give a residue. The residue was purified by prep-HPLC (column: Waters Xbridge 150×25 mm, 5 μm; mobile phase: (0.05% NH₄OH) A—water, B—ACN; gradient 38%-58% B, 10 min) to give FRPPO-050 (4.03 mg, 7.42 μmol, 4.83% yield, 95.8% purity) as a yellow solid.

LCMS: RT=0.967 min, m/z=521.2 (M+1)⁺.

HPLC: RT=2.105 min.

¹H NMR: 400 MHz CDCl₃ δ 8.03 (s, 1H), 7.71-7.70 (m, 1H), 7.55-7.53 (m, 1H), 7.21-7.20 (m, 1H), 6.90-6.86 (m, 1H), 6.37 (s, 1H), 6.20-6.13 (m, 2H), 3.73 (s, 3H), 3.61-3.54 (m, 2H), 3.47-3.43 (m, 2H), 2.51-2.40 (m, 2H).

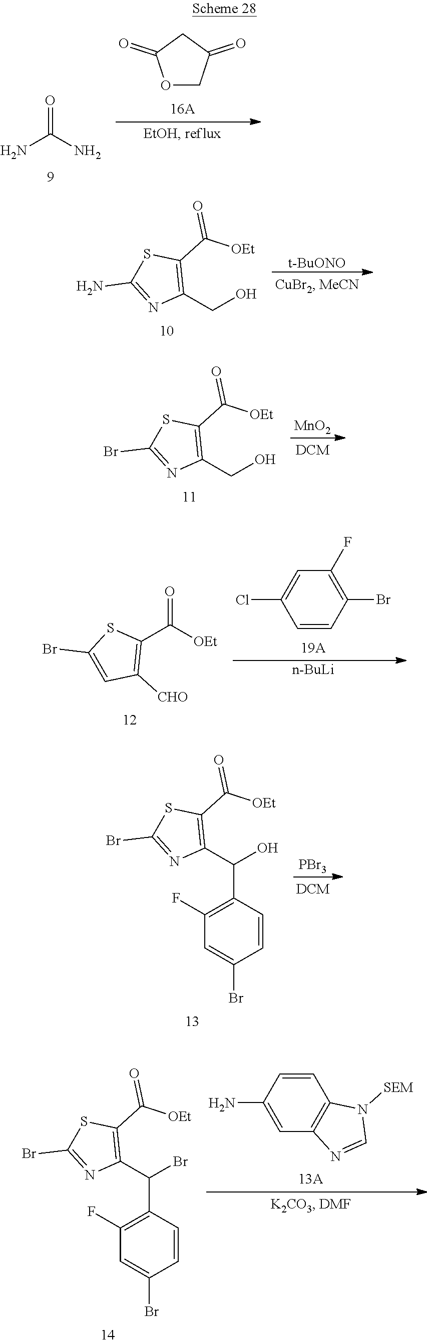

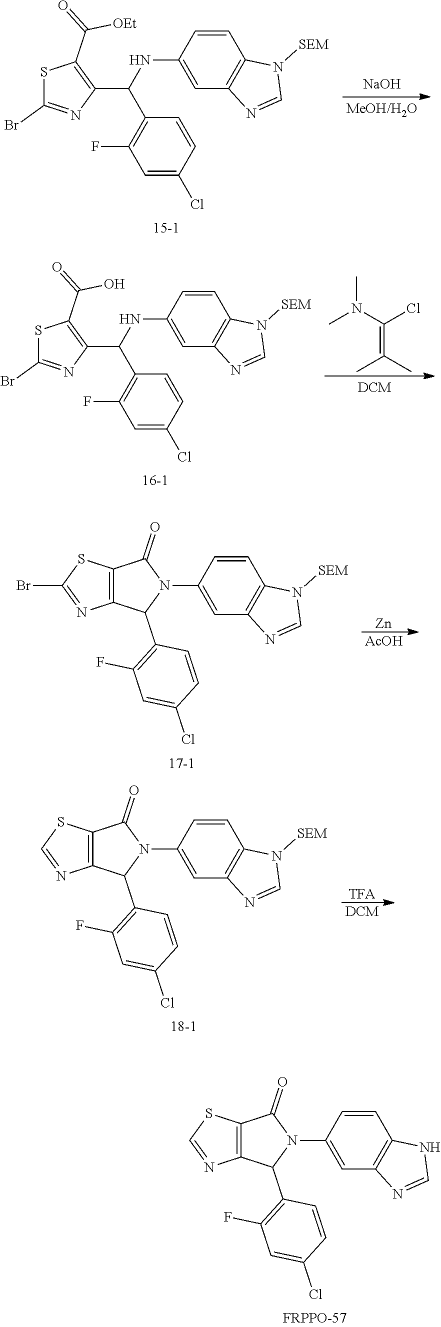

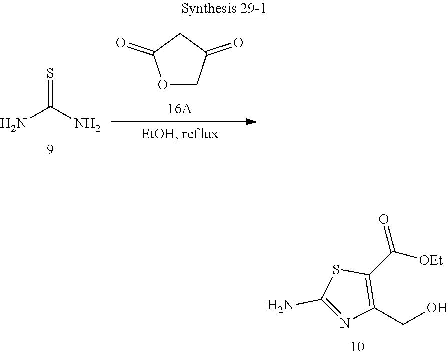

A solution of compound 16A (30.0 g, 223 mmol, 1.00 eq) and compound 9 (20.4 g, 267 mmol, 1.20 eq) in EtOH (300 mL) was stirred at 80° C. for 5 hrs. The mixture was concentrated to give a residue. The residue was dissolved in HCl solution (1 M) (500 mL). The solution was adjusted to pH 8 with Na₂CO₃ solution. The solid was filtered and concentrated to give compound 10 (32.0 g, 158 mmol, 70.9% yield) as a yellow solid.

¹H NMR: 400 MHz DMSO δ 7.85-7.63 (m, 2H), 4.86-4.83 (m, 1H), 4.56-4.55 (m, 2H), 4.23-4.12 (m, 2H), 1.24-1.20 (m, 3H).

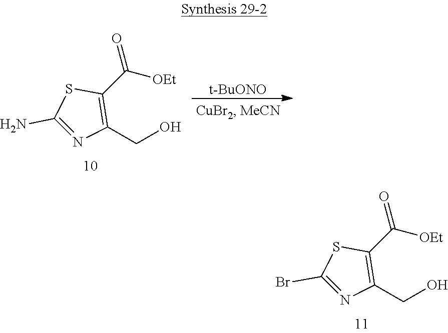 [SMILES: "t-BuONO" ; CCOC(=O)c1sc(N)nc1CO ; CCOC(=O)c1sc(Br)nc1CO]

To a solution of ethyl ester 10 (20.0 g, 98.9 mmol, 1.00 eq) and CuBr₂ (26.5 g, 119 mmol, 5.56 mL, 1.20 eq) in MeCN (200 mL) was added tert-butyl nitrite (15.3 g, 148 mmol, 17.7 mL, 1.50 eq) at 0° C. The mixture was stirred at 25° C. for 10 hrs. NH₄Cl solution (500 mL) was added to the mixture and extracted with EtOAc (300 mL×2). The combined organic layers were dried over anhydrous sodium sulfate, filtrated and concentrated to give compound 11 (15.0 g, crude) as brown oil.

¹H NMR: 400 MHz CDCl₃ δ 4.97 (s, 2H), 4.37-4.26 (m, 2H), 3.67 (s, 1H), 1.41-1.32 (m, 3H).

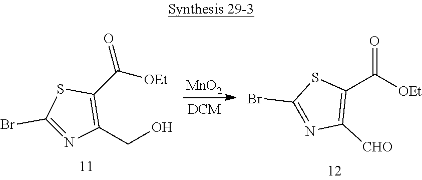

To a solution of compound 11 (14.5 g, 54.5 mmol, 1.00 eq) in DCM (120 mL) was added PCC (29.0 g, 135 mmol, 2.47 eq). The mixture was stirred at 15° C. for 20 hrs. The mixture was filtered and the filtrate was concentrated to give compound 279_4 (8.52 g, 28.7 mmol, 52.7% yield) as a yellow oil.

¹H NMR: 400 MHz CDCl₃ δ 10.4 (s, 1H), 4.41-4.32 (m, 2H), 1.37-1.33 (m, 3H).

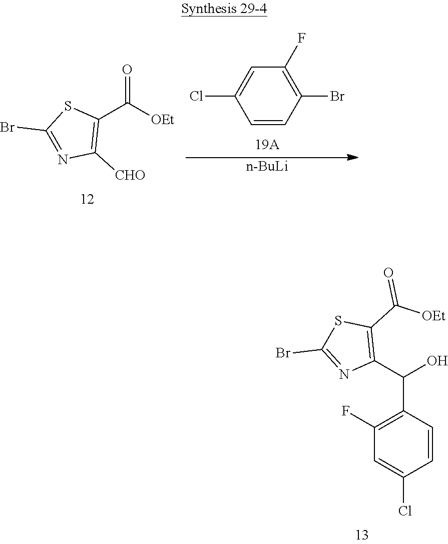

To a solution of compound 19A (10.0 g, 47.7 mmol, 1.00 eq) in THF (100 mL) was added i-PrMgBr (1.00 M, 47.7 mL, 1.00 eq) at 0° C., the mixture was stirred at 0° C. for 1 hr, the mixture was added to a solution of compound 12 (6.30 g, 23.9 mmol, 0.5 eq) in THF (50 mL) at 0° C. and the mixture was stirred at 0° C. for 1 hr. The mixture was quenched with water (300 mL) and extracted with EtOAc (300 mL×2). The combined organic layers were dried over anhydrous sodium sulfate, filtrated and concentrated to give a residue. The residue was purified by column chromatography (silica gel, petroleum ether/ethyl acetate=50/1 to 5/1) to give compound 13 (4.10 g, crude) as a yellow oil.

¹H NMR: 400 MHz CDCl₃ δ 7.46-7.44 (m, 1H), 7.16-7.13 (m, 1H), 7.09-7.06 (m, 1H), 6.62 (s, 1H), 4.41-4.34 (m, 2H), 1.38-1.35 (m, 3H).

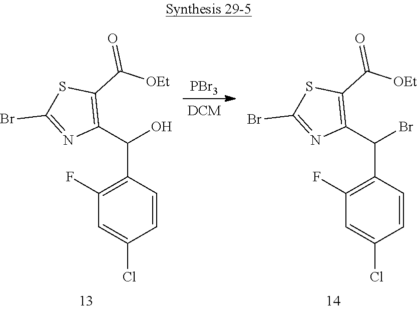

To a solution of compound 13 (1.50 g, 3.80 mmol, 1.00 eq) in DCM (20 mL) was added PBr₃ (5.14 g, 19.0 mmol, 5.00 eq). The mixture was stirred at 25° C. for 1 hr. The mixture was diluted with DCM (30 mL) and washed with water (20 mL), brine (20 mL), dried over anhydrous sodium sulfate, filtered and concentrated to give compound 14 (1.50 g, crude) as a yellow oil, which was used in the next step directly.

LCMS: RT=1.068 min, m/z=457.8 (M+1)⁺.

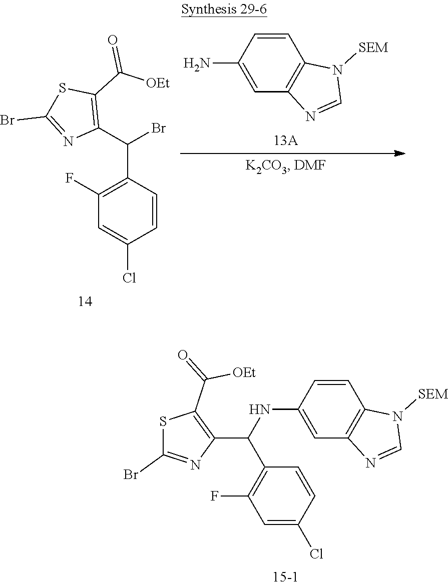 [SMILES: CCOC(=O)c1sc(Br)nc1C(Br)c1ccc(Cl)cc1F ; C[Si](C)(C)CCOCn1cnc2cc(N)ccc21 ; CCOC(=O)c1sc(Br)nc1C(Nc1ccc2c(c1)ncn2COCC[Si](C)(C)C)c1ccc(Cl)cc1F]

To a solution of compound 14 (1.50 g, 3.28 mmol, 1.00 eq) and compound B (900 mg, 3.42 mmol, 1.04 eq) in DMF (20 mL) was added K₂CO₃ (1.50 g, 10.8 mmol, 3.31 eq). The mixture was stirred at 80° C. for 1 hr. The mixture was diluted with EtOAc (200 mL) and washed with water (200 mL), brine (200 mL), dried over anhydrous sodium sulfate, filtrated and concentrated to give a residue. The crude product was purified by reversed-phase HPLC (0.1% formic acid H₂O/MeCN) to give compound 15-1 (1.60 g, crude) as a yellow oil.

LCMS: RT=0.826 min, m/z=641.0 (M+1)⁺.

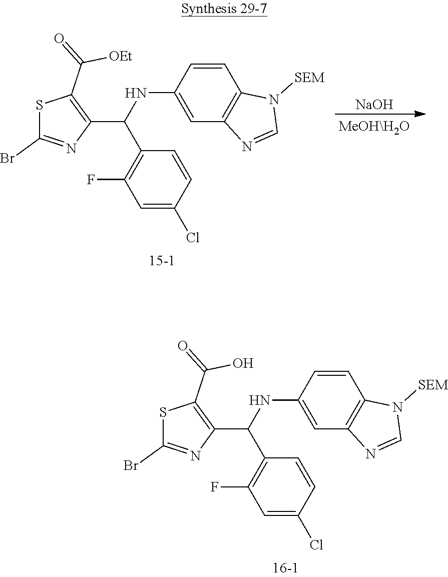 [SMILES: CCOC(=O)c1sc(Br)nc1C(Nc1ccc2c(c1)ncn2COCC[Si](C)(C)C)c1ccc(Cl)cc1F ; "NaOH" ; C[Si](C)(C)CCOCn1cnc2cc(NC(c3ccc(Cl)cc3F)c3nc(Br)sc3C(=O)O)ccc21]

To a solution of compound 15-1 (1.60 g, 2.50 mmol, 1.00 eq) in MeOH (20 mL) and H₂O (4 mL) was added NaOH (2 M, 3.75 mL, 3.00 eq). The mixture was stirred at 25° C. for 0.5 hr. The mixture was concentrated under reduced pressure to give a residue. The residue was diluted in water (100 mL) and adjusted to pH 5 with HCl solution (1 M). The mixture was extracted with EtOAc (50 mL×2). The combined organic layers were dried over anhydrous sodium sulfate, filtrated and concentrated to give compound 16-1 (1.40 g, crude) as a yellow solid, which was used in the next step directly.

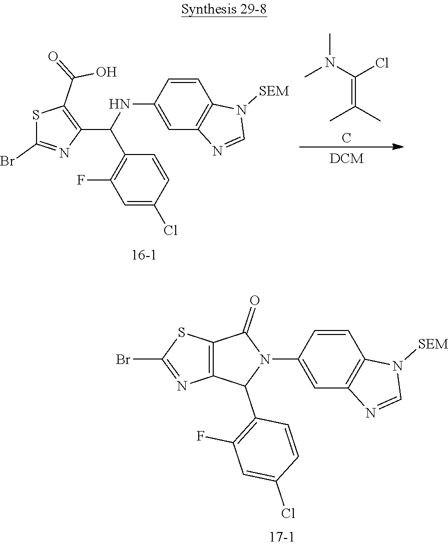

To a solution of compound 16-1 (1.40 g, 2.29 mmol, 1.00 eq) in DCM (20 mL) was added compound C (1.20 g, 8.98 mmol, 1.19 mL, 3.93 eq) at 0° C. The mixture was stirred at 0° C. for 2 hrs. The mixture was concentrated under reduced pressure to give a residue. The residue was purified by reversed-phase HPLC (0.1% formic acid H₂O/MeCN) to give compound 17-1 (550 mg, crude) as a yellow solid, which was used in the next step directly without further purification.

LCMS: RT=0.898 min, m/z=595.1 (M+1)⁺.

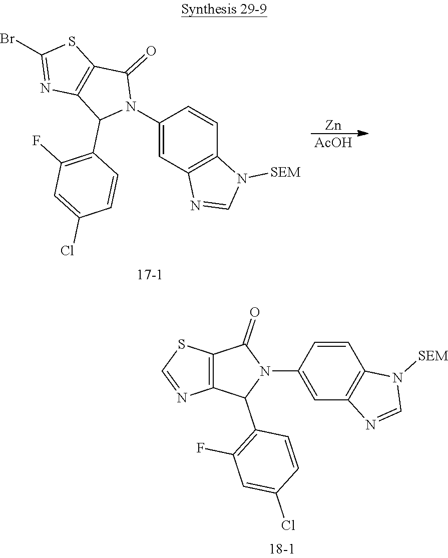

To a solution of compound 17-1 (200 mg, 337 μmol, 1.00 eq) in AcOH (5 mL) was added Zn (200 mg, 3.06 mmol, 9.08 eq). The mixture was stirred at 25° C. for 2 hrs. LCMS showed desired mass (RT=0.878 min, m/z=515.0) was detected. The mixture was filtrated and the filtrate was concentrated under reduced pressure to give a residue. The residue was purified by reversed-phase HPLC (0.1% formic acid H₂O/MeCN) to give compound 18-1 (100 mg, crude) as yellow oil.

LCMS: RT=0.878 min, m/z=515.0 (M+1)⁺.

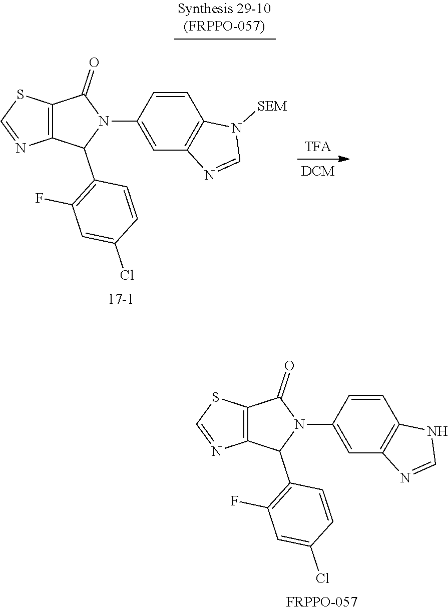 [SMILES: C[Si](C)(C)CCOCn1cnc2cc(N3C(=O)c4scnc4C3c3ccc(Cl)cc3F)ccc21 ; O=C1c2scnc2C(c2ccc(Cl)cc2F)N1c1ccc2[nH]cnc2c1]

To a solution of compound 17-1 (50 mg, 97.1 μmol, 1.00 eq) in DCM (5 mL) was added TFA (1.54 g, 13.5 mmol, 1 mL, 139 eq). The mixture was stirred at 25° C. for 1 hr. The reaction mixture was adjusted to pH 8 with Na₂CO₃ (aq), then extracted with DCM (20 mL×2). The combined organic layers were dried over Na₂SO₄, filtered and concentrated under reduced pressure to give a residue. The residue was purified by prep-HPLC (column Waters Xbridge 150×25 mm, 5 μm; mobile phase: (0.05% NH₄OH) A—water, B—ACN; gradient 18%-48% B, 10 min) to give FRPPO-057 (9.50 mg, 23.8 μmol, 24.5% yield, 96.2% purity) as a yellow solid.

LCMS: RT=0.864 min, m/z=385.1 (M+1)⁺.

HPLC: RT=1.610 min.

¹H NMR: 400 MHz CDCl₃ δ 9.11 (s, 1H), 8.05-8.02 (m, 1H), 7.79-7.78 (m, 1H), 7.61-7.53 (m, 2H), 7.09-70.6 (m, 1H), 7.02-6.96 (m, 2H), 6.53 (s, 1H).

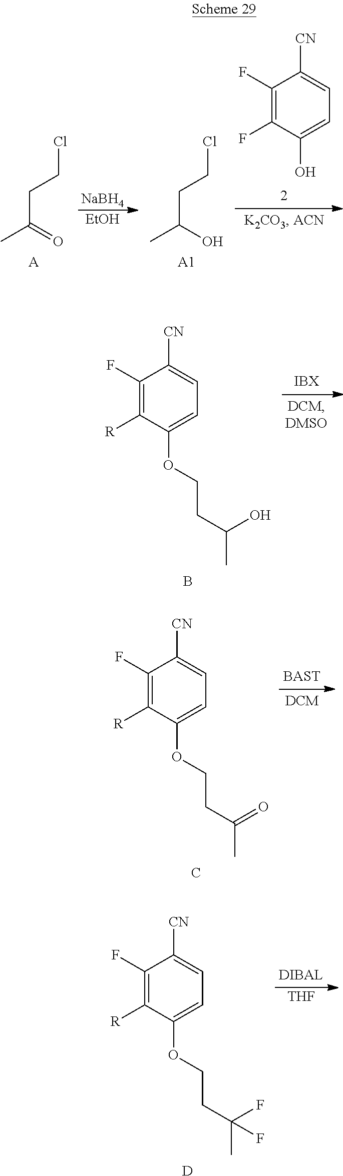

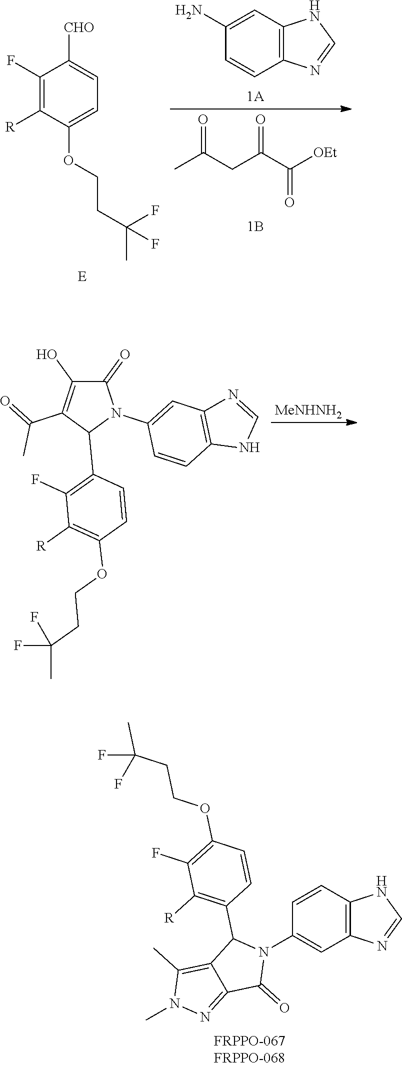

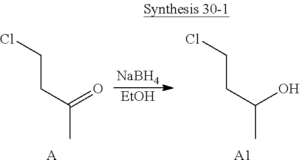

To a solution of compound A (10.0 g, 93.85 mmol, 1.00 eq) in EtOH (50 mL) was added NaBH₄ (4.19 g, 110 mmol, 1.18 eq). The mixture was stirred at 25° C. for 24 h. The reaction mixture was partitioned between water 100 mL and ethyl acetate 150 mL. The organic phase was separated, washed with brine 100 mL, dried over Na₂SO₄, filtered and concentrated under reduced pressure to give compound A-1 (5.00 g, 46.0 mmol, 49.0% yield) as colorless oil.

¹H NMR: 400 MHz CDCl₃ δ 4.08-4.05 (m, 1H), 3.73-3.63 (m, 2H), 1.94-1.87 (m, 2H), 1.26-1.25 (m, 3H).

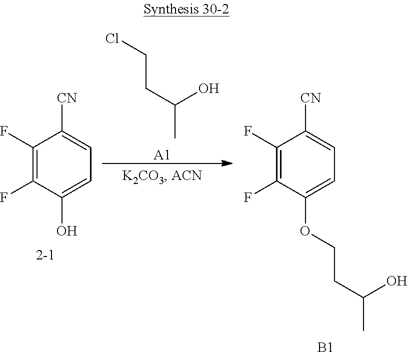

To a solution of compound 2-1 (7.07 g, 45.5 mmol, 0.99 eq) in ACN (100 mL) was added K₂CO₃ (12.7 g, 92.1 mmol, 2.00 eq) and compound A1 (5.00 g, 46.05 mmol, 1.00 eq). The mixture was stirred at 90° C. for 12 hrs. The reaction mixture was concentrated under reduced pressure to remove ACN. The residue was diluted with water 50 mL and extracted with Ethyl acetate 90 mL (30 mL×3). The combined organic layers were washed with brine 50 mL, dried over Na₂SO₄, filtered and concentrated under reduced pressure to give a residue. The residue was purified by flash silica gel chromatography (ISCO; 20 g SepaFlash Silica Flash Column, Eluent of 0-100% ethyl acetate/petroleum ether gradient @ 40 mL/min), to give compound B1 (5.00 g, 22.0 mmol, 47.7% yield) as a colorless oil.

¹H NMR: 400 MHz CDCl₃ δ 7.36-7.32 (m, 1H), 6.87-6.84 (m, 1H), 4.32-4.29 (m, 1H), 4.25-4.22 (m, 1H), 3.71-3.64 (m, 1H), 1.96-1.86 (m, 2H), 1.31-1.29 (m, 3H).

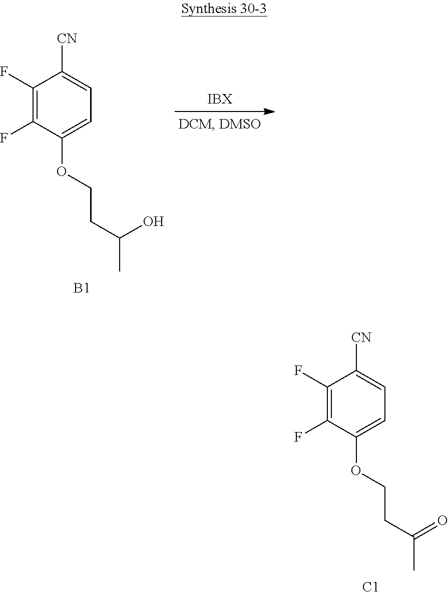

To a solution of compound B1 (5.00 g, 22.01 mmol, 1.00 eq) in DMSO (100 mL) was added IBX (18.4 g, 66.0 mmol, 3.00 eq) and DCM (10 mL). The mixture was stirred at 25° C. for 12 hrs. The reaction mixture was partitioned between water 100 mL and DCM 200 mL. The organic phase was separated, washed with brine 100 mL, dried over Na₂SO₄, filtered and concentrated under reduced pressure to give a residue. The residue was purified by flash silica gel chromatography (ISCO; 120 g SepaFlash Silica Flash Column, Eluent of 0-100% ethyl acetate/petroleum ether gradient @ 40 mL/min), to give compound C1 (4.00 g, 17.7 mmol, 80.7% yield) as a colorless oil.

¹H NMR 400 MHz CDCl₃ δ 7.36-7.32 (m, 1H), 7.27-7.19 (m, 1H), 4.38-4.35 (m, 2H), 3.03-3.01 (m, 2H), 2.27 (s, 3H).

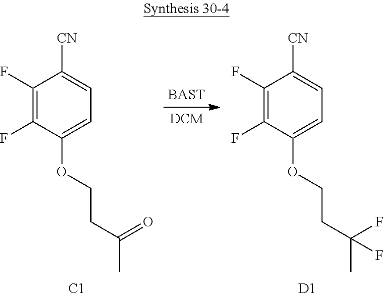 [SMILES: CC(=O)CCOc1ccc(C#N)c(F)c1F ; CC(F)(F)CCOc1ccc(C#N)c(F)c1F ; "C1" ; "D1"]

To a solution of BAST (15.1 g, 68.48 mmol, 15 mL, 5.14 eq) in DCM (30 mL) was added compound C1 (3.00 g, 13.3 mmol, 1.00 eq) at 0° C. The mixture was stirred at 50° C. for 2 hrs. The reaction mixture was poured into ice water and extracted with DCM 600 mL (200 mL×3). The combined organic layers were washed with NaHCO₃ and brine, dried over Na₂SO₄, filtered and concentrated under reduced pressure to give a residue. The residue was purified by flash silica gel chromatography (ISCO; 80 SepaFlash Silica Flash Column, Eluent of 0-100% ethyl acetate/petroleum ether gradient @60 mL/min) to give compound D1 (1.20 g, 4.85 mmol, 36.4% yield) as a yellow oil.

¹H NMR: 400 MHz CDCl₃ δ 7.38-7.34 (m, 1H), 6.87-6.84 (m, 1H), 4.34-4.31 (m, 2H), 2.52-2.43 (m, 2H), 1.77-1.68 (m, 3H).

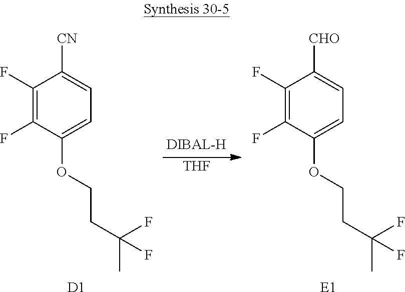 [SMILES: CC(F)(F)CCOc1ccc(C#N)c(F)c1F ; CC(F)(F)CCOc1ccc(C=O)c(F)c1F ; "D1" ; "E1" ; "THF"]

To a solution of compound D1 (1.00 g, 4.05 mmol, 1.00 eq) in THF (10 mL) was added DIBAL-H (1 M, 8.09 mL, 2.00 eq) at −70° C., the mixture was stirred at 25° C. for 3 hrs. The reaction mixture was quenched by addition NH₄Cl 10 mL at 25° C., and then diluted with water 100 mL and filtered and extracted with ethyl acetate 150 mL (50 mL×3). The combined organic layers were washed with brine 100 mL, dried over Na₂SO₄, filtered and concentrated under reduced pressure to give a residue. The crude product was purified by reversed-phase HPLC (0.1% formic acid H₂O/MeCN) to give compound E1 (350 mg, 1.40 mmol, 34.58% yield) as a yellow oil.

¹H NMR: 400 MHz CDCl₃ δ 10.2 (s, 1H), 7.71-7.61 (m, 1H), 6.89-6.85 (m, 1H), 4.36-4.29 (m, 2H), 2.51-2.42 (m, 2H), 1.78-1.69 (m, 3H).

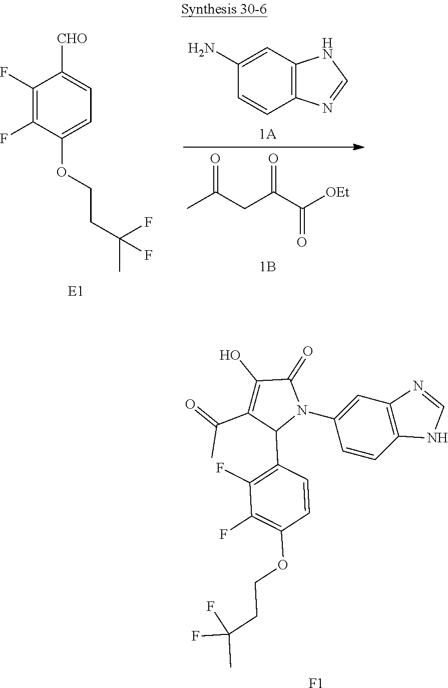

To a solution of compound E1 (350 mg, 1.40 mmol, 1.00 eq) and compound 1A (186 mg, 1.40 mmol, 1.00 eq) in EtOH (10 mL) was added AcOH (88.2 mg, 1.47 mmol, 84 uL, 1.05 eq). The mixture was stirred at 25° C. for 0.5 hrs, Then compound 1B (224 mg, 1.42 mmol, 200 uL, 1.01 eq) was added, the mixture was stirred at 85° C. for 0.5 hrs. The reaction mixture was adjusted to pH 8 with saturated NaHCO₃ and extracted with ethyl acetate 150 mL (50 mL×3). The combined organic layers were washed with brine 50 mL, dried over Na₂SO₄, filtered and concentrated under reduced pressure to give compound F1 (670 mg, crude) as a white solid.

LCMS: RT=0.623 min. m/z=478.2 (M+1)⁺.

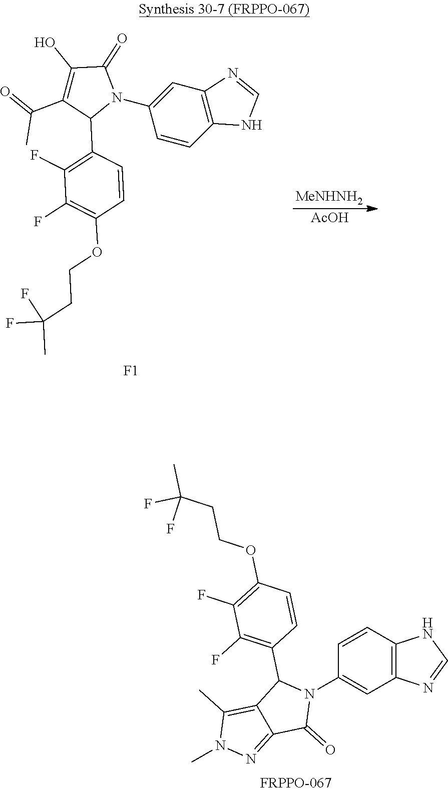 [SMILES: "MeNHNH2" ; "AcOH" ; CC(=O)C1=C(O)C(=O)N(c2ccc3[nH]cnc3c2)C1c1ccc(OCCC(C)(F)F)c(F)c1F ; "F1" ; Cc1c2c(nn1C)C(=O)N(c1ccc3[nH]cnc3c1)C2c1ccc(OCCC(C)(F)F)c(F)c1F]

To a solution of compound F1 (670 mg, 1.40 mmol, 1.00 eq) in AcOH (10 mL) was added methylhydrazine (131 mg, 2.85 mmol, 150 uL, 2.03 eq). The mixture was stirred at 85° C. for 2 h. The reaction mixture was adjusted to pH 9 with saturated NaHCO₃, diluted with water 50 mL and extracted with DCM 200 mL (100 mL×2). The combined organic layers were washed with brine 50 mL, dried over Na₂SO₄, filtered and concentrated under reduced pressure to give a residue. The residue was purified by prep-HPLC (column: Waters Xbridge 150×25 mm, 5 μm; mobile phase: (0.05% NH₄OH) A—water, B—ACN; gradient 34%-54% B, 10 min) to give FRPPO-067 (65.97 mg, 135 μmol, 9.64% yield, 100% purity) as an off-white solid.

LCMS: RT=0.924 min, m/z=488.3 (M+1)⁺.

HPLC: RT=1.879 min.

¹H NMR: 400 MHz DMSO δ 12.4 (s, 1H), 8.17-8.16 (m, 1H), 7.62-7.56 (m, 1H), 7.54-7.42 (m, 1H), 7.31-7.19 (m, 1H), 6.55-6.52 (m, 1H), 6.52-6.16 (m, 2H), 3.87 (s, 3H), 3.62-3.55 (m, 2H), 3.31-3.29 (m, 2H), 2.44-2.38 (m, 2H), 2.12 (s, 3H).

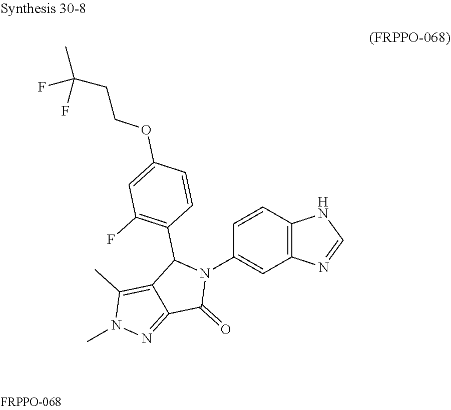 [SMILES: Cc1c2c(nn1C)C(=O)N(c1ccc3[nH]cnc3c1)C2c1ccc(OCCC(C)(F)F)cc1F]

FRPPO-068 (6.48 mg, 13.7 μmol, 5.25% yield, 99.3% purity) was obtained using an analogous method as a white solid.

LCMS: RT=0.878 min, m/z=470.1 (M+1)⁺.

HPLC: RT=2.176 min.

¹H NMR: EW18380-11-P1A1 400 MHz CDCl₃ δ 7.96 (s, 1H), 7.81 (s, 1H), 7.55-7.52 (d, J=12, 1H), 7.23-7.21 (m, 1H), 6.94-6.90 (m, 1H), 6.54-6.50 (m, 2H), 0.6.34 (s, 1H), 4.05-4.02 (m, 2H), 3.94 (s, 3H), 2.36-2.25 (m, 2H), 2.18 (s, 3H), 1.69-1.60 (q, 3H).

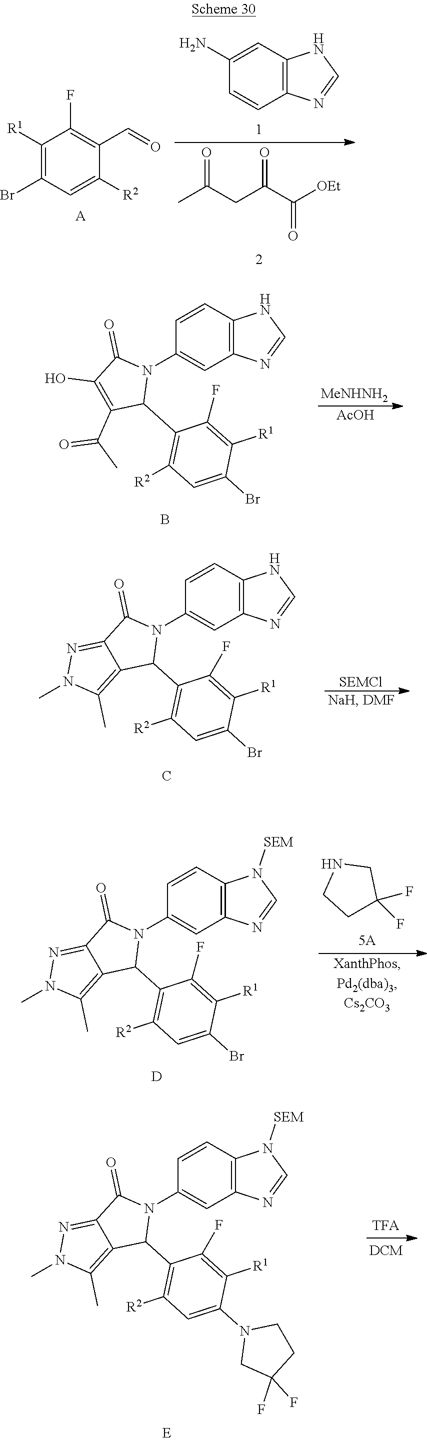

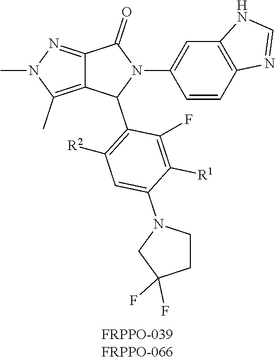

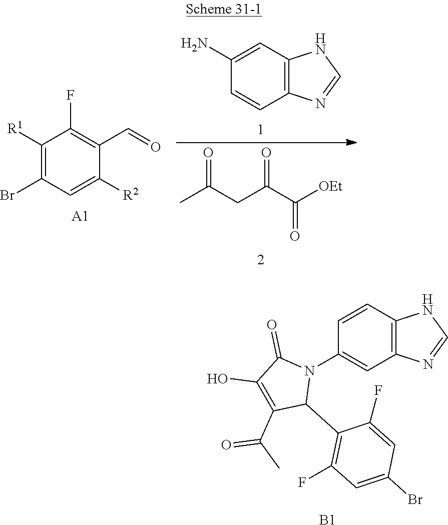

To a solution of compound A1 (5.00 g, 22.6 mmol, 1.00 eq) and compound A (3.01 g, 22.6 mmol, 1.00 eq) in EtOH (25 mL) was added AcOH (1.36 g, 22.6 mmol, 1.29 mL, 1.00 eq). The mixture was stirred at 25° C. for 0.5 hrs. Then compound 2 (3.58 g, 22.62 mmol, 3.19 mL, 1 eq) was added, the mixture was stirred at 85° C. for 0.5 hrs. The reaction mixture was filtered and concentrated under reduced pressure to give crude compound B1 (10.0 g, 22.3 mmol, 98.6% yield) as a white solid, which was used into next step directly.

LCMS: RT=0.619 min, m/z=450.2 (M+1)⁺.

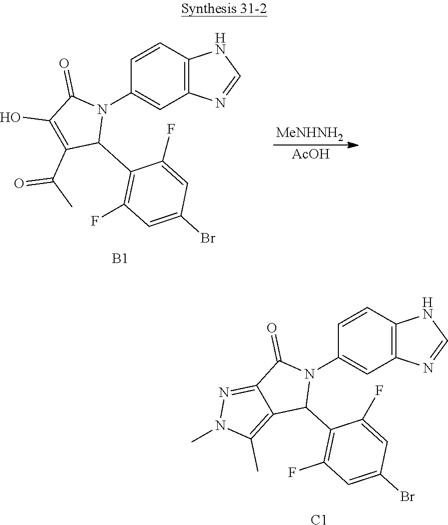

To a solution of compound B1 (10.0 g, 22.3 mmol, 1.00 eq) in AcOH (100 mL) was added methylhydrazine (2.57 g, 22.3 mmol, 2.94 mL, 1.00 eq). The mixture was stirred at 90° C. for 2 hrs. The reaction mixture was concentrated under reduced pressure to remove AcOH. The residue was diluted with water 200 mL and adjusted to pH 8 with Na₂CO₃, then extracted with DCM 600 mL (200 mL×3). The combined organic layers were washed with brine 300 mL, dried over Na₂SO₄, filtered and concentrated under reduced pressure to give a residue. The residue was purified by prep-HPLC (column: Phenomenex Gemini C18 250×50 mm, 10 μm; mobile phase: (0.05% NH₄OH) A—water, B—ACN; gradient 22%-47% B, 22 min) to give compound C1 (4 g, 8.73 mmol, 39.13% yield) as a white solid.

¹H NMR: 400 MHz DMSO δ 12.5 (s, 1H), 8.19 (s, 1H), 7.66 (s, 1H), 7.51-7.27 (m, 4H), 6.72 (s, 1H), 3.89 (s, 3H), 2.15 (s, 3H).

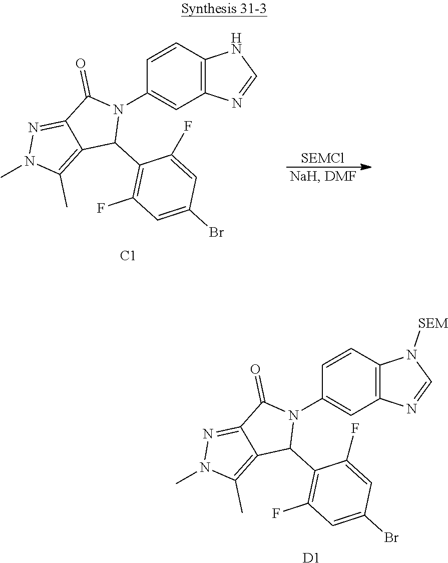

To a solution of compound C1 (2.00 g, 4.36 mmol, 1.00 eq) in DMF (20 mL) was added NaH (350 mg, 8.75 mmol, 60% purity, 2.01 eq) and SEMCl (1.09 g, 6.55 mmol, 1.16 mL, 1.50 eq) at 0° C. The mixture was stirred at 25° C. for 12 hrs. The reaction mixture was partitioned between water 100 mL and ethyl acetate 150 mL. The organic phase was separated, washed with brine 100 mL, dried over Na₂SO₄, filtered and concentrated under reduced pressure to give a residue. The crude product was purified by reversed-phase HPLC (0.1% NH₄OH H₂O/MeCN) to give compound D1 (1.20 g, 2.04 mmol, 46.7% yield) as a yellow solid.

¹H NMR: 400 MHz CDCl₃ δ 7.95-7.94 (m, 1H), 7.94-7.73 (m, 1H), 7.72-7.61 (m, 1H), 7.60-7.58 (m, 1H), 7.57-7.46 (m, 1H), 7.29-7.28 (m, 1H), 5.48 (s, 1H), 3.94 (s, 3H), 3.49-3.46 (m, 2H), 2.18 (s, 3H), 0.89-0.87 (m, 2H), 0.01-0.007 (m, 11H).

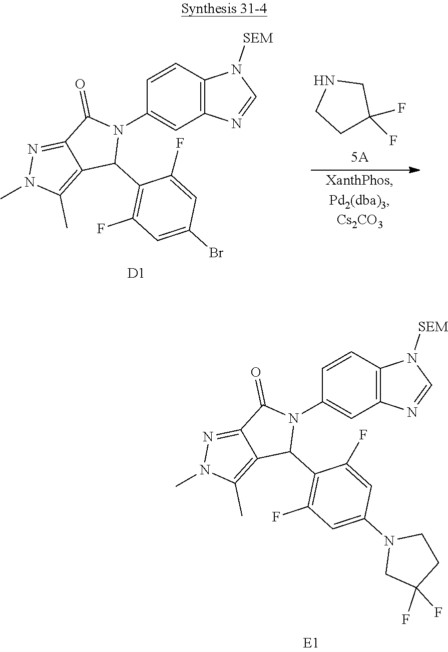

To a solution of compound D1 (1.00 g, 1.70 mmol, 1.00 eq) and compound 5A (400 mg, 2.79 mmol, 1.64 eq, HCl) in dioxane (10 mL) was added XantPhos (100 mg, 172 μmol, 0.01 eq) and Pd₂(dba)₃ (160 mg, 174 μmol, 0.01 eq) and Cs₂CO₃ (1.25 g, 3.84 mmol, 2.26 eq). The mixture was stirred at 100° C. for 12 hrs. The reaction mixture was partitioned between water 100 mL and ethyl acetate 200 mL. The organic phase was separated, washed with brine 100 mL, dried over Na₂SO₄, filtered and concentrated under reduced pressure to give a residue. The residue was purified by reversed-phase HPLC (0.1% NH₄OH H₂O/MeCN) to give compound E1 (500 mg) as a yellow solid, which was used in the next step directly.

LCMS: RT=1.108 min, m/z=615.4 (M+1)⁺.

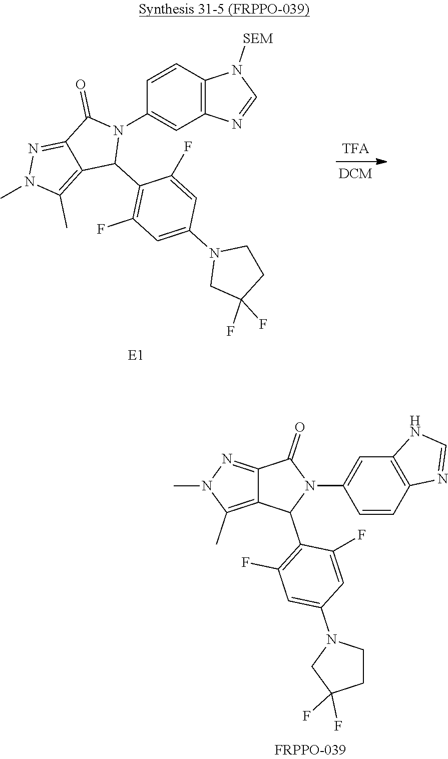 [SMILES: Cc1c2c(nn1C)C(=O)N(c1ccc3c(c1)ncn3COCC[Si](C)(C)C)C2c1c(F)cc(N2CCC(F)(F)C2)cc1F ; Cc1c2c(nn1C)C(=O)N(c1ccc3nc[nH]c3c1)C2c1c(F)cc(N2CCC(F)(F)C2)cc1F]

To a solution of compound E1 (200 mg, 325 μmol, 1.00 eq) in DCM (4 mL) was added TFA (1.54 g, 13.5 mmol, 1 mL, 41.5 eq). The mixture was stirred at 25° C. for 0.5 hr. The reaction mixture was partitioned between water 100 mL and DCM 150 mL. The organic phase was separated, washed with brine 50 mL, dried over Na₂SO₄, filtered and concentrated under reduced pressure to give a residue. The residue was purified by prep-HPLC (column: Xtimate C18 150 mm×80 mm, 10 μm; mobile phase: (0.05% NH₄OH) A—water, B—ACN; gradient 34%-54% B, 10 min) to give FRPPO-039 (4.07 mg, 8.38 μmol, 2.58% yield, 99.8% purity) as a white solid.

LCMS: RT=0.881 min, m/z=485.4 (M+1)⁺.

HPLC: RT=1.845 min.

¹H NMR: 400 MHz DMSO δ 12.4 (s, 1H), 8.17-8.16 (m, 1H), 7.62-7.56 (m, 1H), 7.54-7.42 (m, 1H), 7.31-7.19 (m, 1H), 6.55-6.52 (m, 1H), 6.52-6.16 (m, 2H), 3.87 (s, 3H), 3.62-3.55 (m, 2H), 3.31-3.29 (m, 2H), 2.44-2.38 (m, 2H), 2.12 (s, 3H).

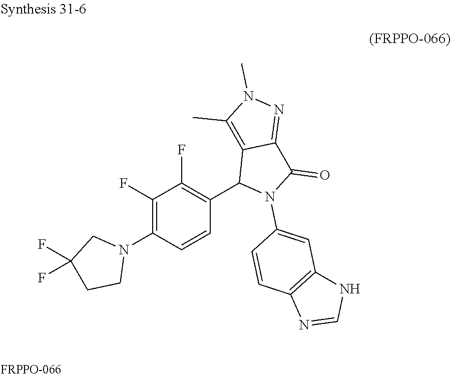 [SMILES: Cc1c2c(nn1C)C(=O)N(c1ccc3nc[nH]c3c1)C2c1ccc(N2CCC(F)(F)C2)c(F)c1F]

FRPPO-066 (70.03 mg, 143 μmol, 14.6% yield, 99.2% purity) was obtained using an analogous method as a yellow solid.

LCMS: RT=0.903 min, m/z=485.4 (M+1)⁺.

HPLC: RT=1.888 min.

¹H NMR: 400 MHz DMSO δ 10.1 (s, 1H), 8.18-8.16 (m, 1H), 8.15-8.12 (m, 1H), 7.53-7.46 (m, 2H), 7.09-7.05 (m, 1H), 6.56-6.54 (m, 1H), 6.52-6.27 (m, 1H), 3.86 (s, 3H), 3.83-3.74 (m, 2H), 3.71-3.50 (m, 2H), 2.45-2.38 (m, 2H), 2.28 (s, 3H).

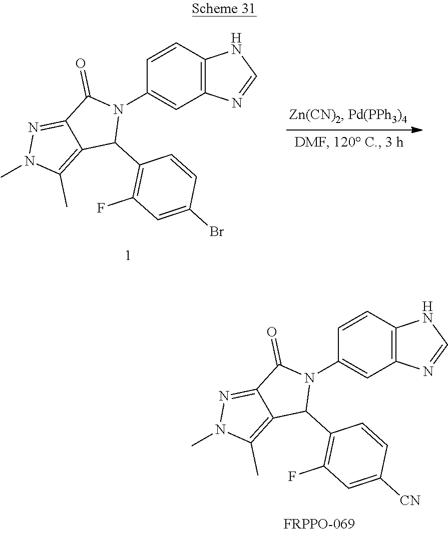 [SMILES: Cc1c2c(nn1C)C(=O)N(c1ccc3[nH]cnc3c1)C2c1ccc(Br)cc1F ; Cc1c2c(nn1C)C(=O)N(c1ccc3[nH]cnc3c1)C2c1ccc(C#N)cc1F]

### Synthesis 32-1

### FRPPO-069

To a solution of Pd(PPh₃)₄ (79 mg, 68.1 μmol, 0.100 eq) and compound 1 (300 mg, 681 μmol, 1.00 eq) in DMF (5 mL) was added Zn(CN)₂ (80 mg, 681 μmol, 43.3 uL, 1.00 eq), the mixture was stirred at 120° C. for 3 h. The reaction mixture was diluted with water (20 mL), and extracted with DCM (30 mL×3). The combined organic layers were washed with saturated salt solution (10 mL×3), dried over Na₂SO₄, filtered and concentrated under reduced pressure to give the residue which was purified by prep-HPLC (column: Waters Xbridge 150×50 mm, 10 μm; mobile phase: (10 mM NH₄HCO₃) A—water, B—ACN; gradient 20%-50% B, 10 min) to give compound FRPPO-069 (14.06 mg, 35.7 μmol, 5.24% yield, 98.2% purity) as a yellow solid.

LCMS: RT=0.835 min, m/z=387.2 (M+1)⁺.

HPLC: RT=1.804 min.

¹HNMR: 400 MHz DMSO δ 8.18 (s, 1H), 7.80 (dd, J=1.2, 10.4 Hz, 1H), 7.73 (d, J=2.0 Hz, 1H), 7.61-7.54 (m, 1H), 7.54-7.47 (m, 2H), 7.32 (br d, J=8.0 Hz, 1H), 6.74 (s, 1H), 3.87 (s, 3H), 2.13 (s, 3H).

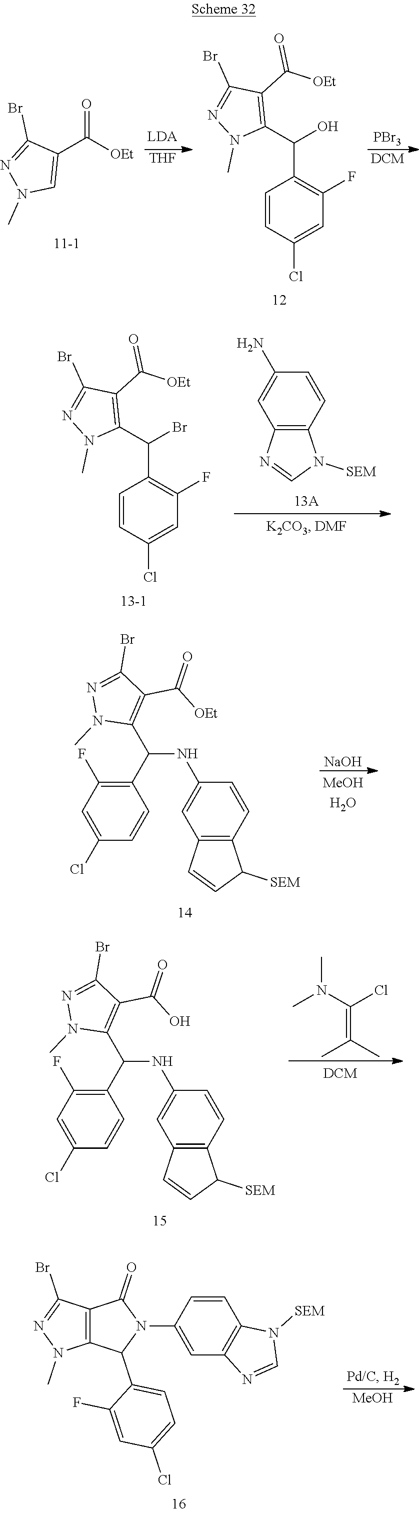

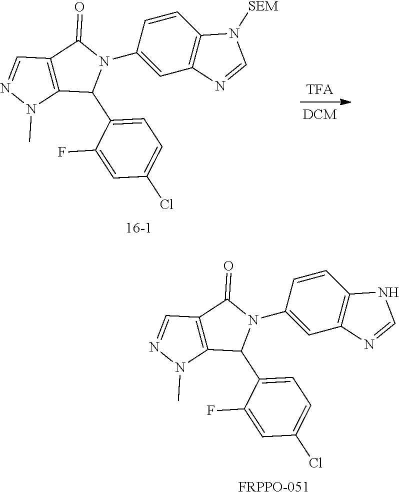 [SMILES: Cn1ncc2c1C(c1ccc(Cl)cc1F)N(c1ccc3c(c1)ncn3COCC[Si](C)(C)C)C2=O ; Cn1ncc2c1C(c1ccc(Cl)cc1F)N(c1ccc3[nH]cnc3c1)C2=O]

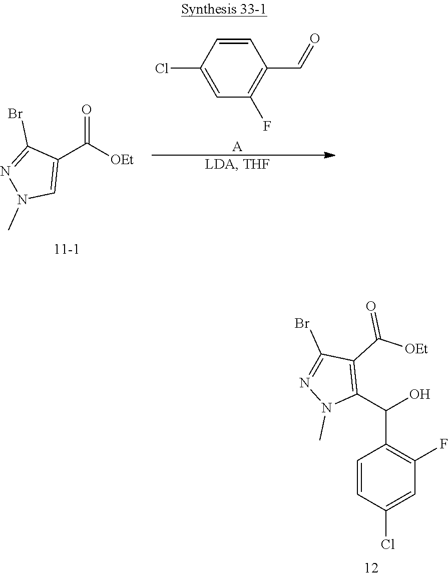

To a solution of compound 11-1 (2.00 g, 8.58 mmol, 1.00 eq) in THF (20 mL) was added LDA (2 M, 13.0 mL, 3.00 eq) at −78° C. The mixture was stirred at −78° C. for 15 min then compound A (2.72 g, 17.2 mmol, 2.00 eq) was added. The mixture was stirred at −78° C. for 15 min. The mixture was poured into NH₄Cl solution (50 mL) and extracted with DCM (50 mL×2). The combined organic layers were dried over anhydrous sodium sulfate, filtered and concentrated to give a residue. The crude product was purified by reversed-phase HPLC (0.1% TFA H₂O/MeCN) to give compound 12 (1.00 g, 2.55 mmol, 29.8% yield) as yellow oil.

¹H NMR: 400 MHz CDCl₃ δ 7.57-7.49 (m, 1H), 7.36-7.27 (m, 1H), 7.17-7.15 (m, 1H), 7.06-7.03 (m, 1H), 6.22 (s, 1H), 4.35-4.28 (m, 2H), 3.90 (s, 3H), 1.39-1.35 (m, 3H).

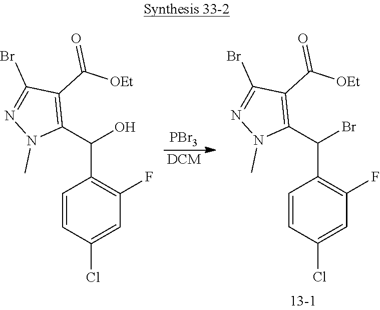

To a solution of compound 12 (1.40 g, 3.57 mmol, 1.00 eq) in DCM (20 mL) was added PBr₃ (4.84 g, 17.9 mmol, 5.00 eq). The mixture was stirred at 25° C. for 6 hrs. The mixture was poured into water (20 mL) and extracted with DCM (20 mL×2). The combined organic layers were dried over anhydrous sodium sulfate, filtered and concentrated to give compound 13-1 (1.50 g, crude) as yellow oil, which was used in the next step without purification.

LCMS: RT=1.074 min, m/z=454.8 (M+1)⁺.

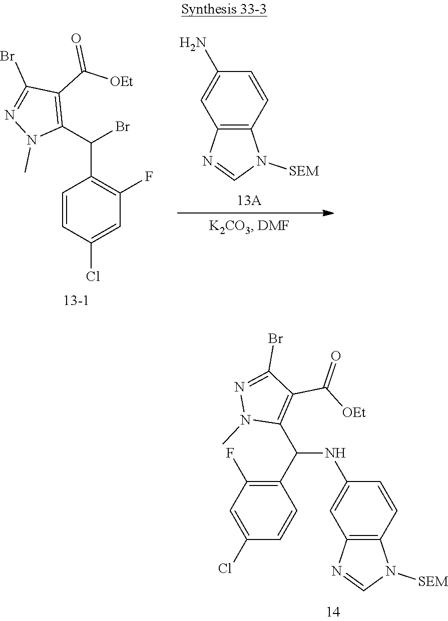 [SMILES: CCOC(=O)c1c(Br)nn(C)c1C(Br)c1ccc(Cl)cc1F ; C[Si](C)(C)CCOCn1cnc2cc(N)ccc21 ; CCOC(=O)c1c(Br)nn(C)c1C(Nc1ccc2c(c1)ncn2COCC[Si](C)(C)C)c1ccc(Cl)cc1F]

To a solution of compound 13-1 (1.50 g, 3.30 mmol, 1.00 eq) and compound 13A (870 mg, 3.30 mmol, 1.00 eq) in DMF (20 mL) was added K₂CO₃ (912 mg, 6.60 mmol, 2.00 eq). The mixture was stirred at 80° C. for 5 hrs. LCMS showed desired mass (RT=0.885 min. m/z=638.0) was detected. Water (50 mL) was added to the mixture and extracted with EtOAc (50 mL×2). The combined organic layers were dried over anhydrous sodium sulfate, filtered and concentrated to give a residue. The residue was purified by reversed-phase HPLC (0.1% TFA H₂O/MeCN) to give compound 14 (900 mg, 1.41 mmol, 42.8% yield, purity: 87.9%) as a yellow oil.

LCMS: RT=0.954 min, m/z=637.9 (M+1)⁺.

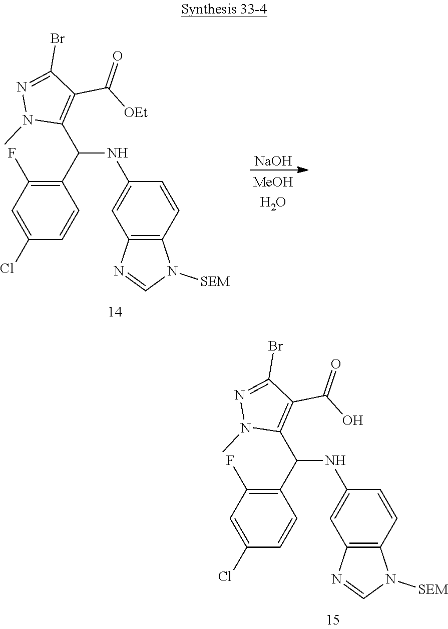 [SMILES: CCOC(=O)c1c(Br)nn(C)c1C(Nc1ccc2c(c1)ncn2COCC[Si](C)(C)C)c1ccc(Cl)cc1F ; "NaOH" ; Cn1nc(Br)c(C(=O)O)c1C(Nc1ccc2c(c1)ncn2COCC[Si](C)(C)C)c1ccc(Cl)cc1F]

To a solution of compound 14 (800 mg, 1.26 mmol, 1.00 eq) in MeOH (10 mL) and H₂O (1 mL) was added NaOH (100 mg, 2.51 mmol, 1.26 mL, 2.00 eq). The mixture was stirred at 25° C. for 12 hrs. The mixture was concentrated under reduced pressure to give a residue. The residue was diluted in water (20 mL) and adjusted to pH 3 with HCl solution (1 M). The mixture was extracted with EtOAc (20 mL×2). The combined organic layers were dried over anhydrous sodium sulfate, filtrated and concentrated to give compound 15 (600 mg, crude) as a yellow solid, which was used in the next step without purification.

LCMS: RT=0.882 min, m/z=609.9 (M+1)⁺.

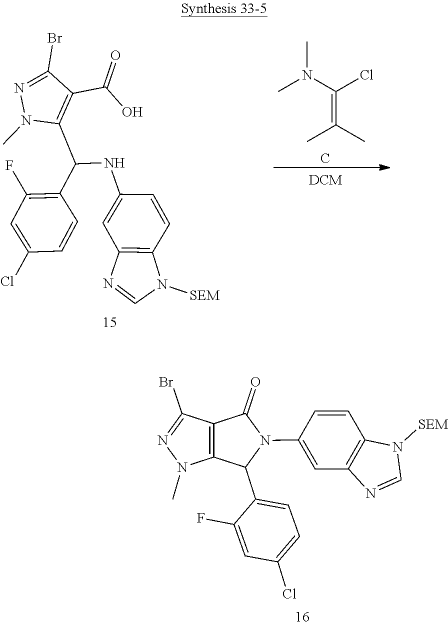

To a solution of compound 15 (600 mg, 985 μmol, 1.00 eq) in DCM (10 mL) was added compound C (360 mg, 2.69 mmol, 356 uL, 2.73 eq) at 0° C. The mixture was stirred at 0° C. for 2 hrs. The mixture was concentrated under reduced pressure to give a residue. The crude product was purified by reversed-phase HPLC (0.1% HCl H₂O/MeCN) to give Compound 16 (360 mg, 609 μmol, 61.8% yield, purity: 98.9%) as a yellow solid.

LCMS: RT=0.944 min, m/z=591.9 (M+1)⁺.

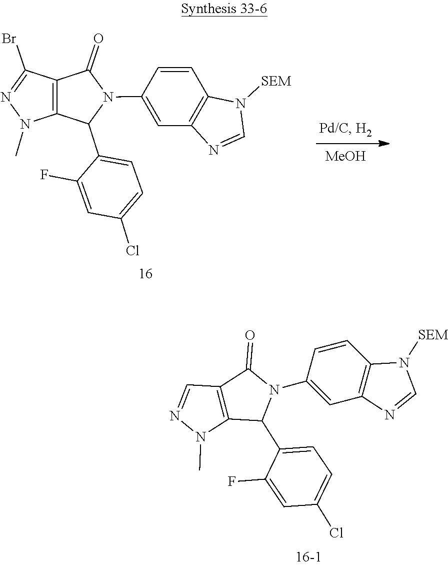 [SMILES: "MeOH" ; Cn1nc(Br)c2c1C(c1ccc(Cl)cc1F)N(c1ccc3c(c1)ncn3COCC[Si](C)(C)C)C2=O ; Cn1ncc2c1C(c1ccc(Cl)cc1F)N(c1ccc3c(c1)ncn3COCC[Si](C)(C)C)C2=O]

To a solution of compound 16 (80 mg, 135 μmol, 1.00 eq) in MeOH (10 mL) was added Pd/C (10 mg, 135.38 μmol, 5% purity, 1 eq). The mixture was stirred under H₂ (15 psi) at 25° C. for 4 hrs. The mixture was filtrated and the filtrate was concentrated under reduced pressure to give compound 16-1 (60 mg, crude) as a yellow oil, which was used in the next step directly without any purification.

LCMS: RT=0.876 min, m/z=512.1 (M+1)⁺.

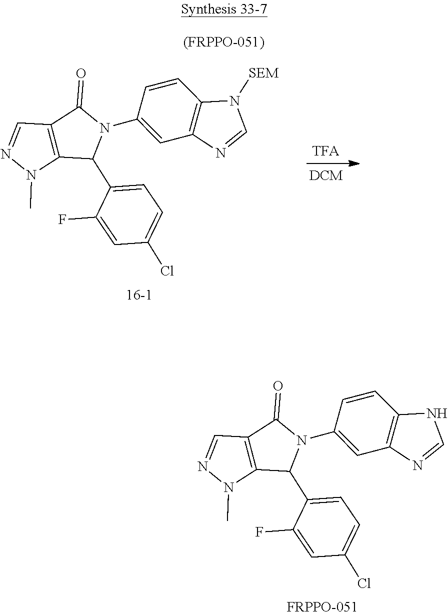 [SMILES: Cn1ncc2c1C(c1ccc(Cl)cc1F)N(c1ccc3c(c1)ncn3COCC[Si](C)(C)C)C2=O ; Cn1ncc2c1C(c1ccc(Cl)cc1F)N(c1ccc3[nH]cnc3c1)C2=O]

To a solution of compound 16-1 (60 mg, 117 μmol, 1.00 eq) in DCM (2 mL) was added TFA (3.08 g, 27.0 mmol, 2 mL, 230 eq). The mixture was stirred at 25° C. for 12 hrs. The mixture was adjusted to pH=8 with Na₂CO₃ solution and extracted with DCM (10 mL×2). The combined organic layers were dried over anhydrous sodium sulfate, filtrated and concentrated to give a residue. The residue was purified by prep-HPLC (column: Waters Xbridge 150×25 mm, 5 μm; mobile phase: (0.05% NH₄OH) A—water, B—ACN; gradient 18%-38% B, 10 min) to give FRPPO-051 (22.94 mg, 60.0 μmol, 51.2% yield, 99.9% purity) as a white solid.

LCMS: RT=0.849 min. m/z=382.2 (M+1)⁺.

HPLC: RT=1.532 min.

¹H NMR: 400 MHz CDCl₃ δ 7.99 (s, 1H), 7.79 (s, 1H), 7.69-7.56 (m, 1H), 7.54-7.53 (m, 1H), 7.12-7.09 (m, 1H), 7.06-7.03 (m, 3H), 6.44 (s, 1H), 3.70 (s, 3H).

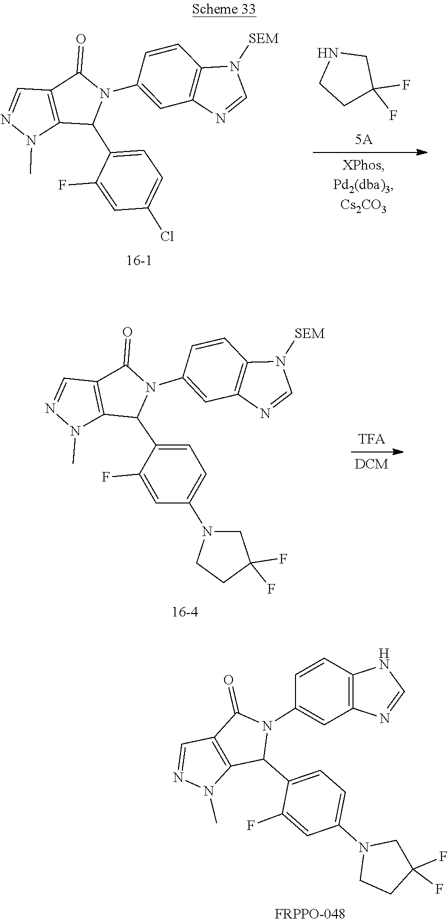 [SMILES: Cn1ncc2c1C(c1ccc(Cl)cc1F)N(c1ccc3c(c1)ncn3COCC[Si](C)(C)C)C2=O ; FC1(F)CCNC1 ; Cn1ncc2c1C(c1ccc(N3CCC(F)(F)C3)cc1F)N(c1ccc3c(c1)ncn3COCC[Si](C)(C)C)C2=O ; Cn1ncc2c1C(c1ccc(N3CCC(F)(F)C3)cc1F)N(c1ccc3[nH]cnc3c1)C2=O]

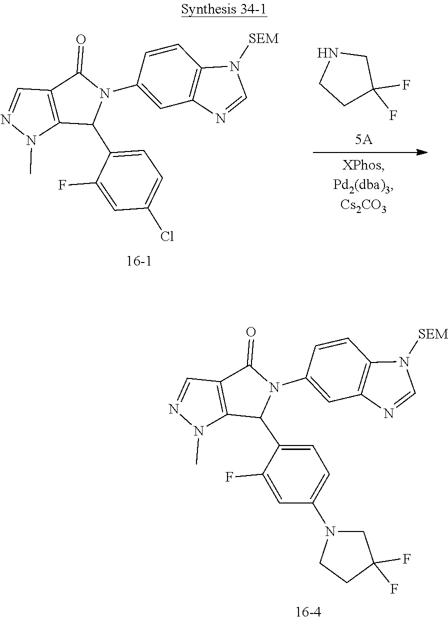 [SMILES: Cn1ncc2c1C(c1ccc(Cl)cc1F)N(c1ccc3c(c1)ncn3COCC[Si](C)(C)C)C2=O ; FC1(F)CCNC1 ; Cn1ncc2c1C(c1ccc(N3CCC(F)(F)C3)cc1F)N(c1ccc3c(c1)ncn3COCC[Si](C)(C)C)C2=O]

To a mixture of compound 16-1 (120 mg, 234 μmol, 164 uL, 1.00 eq) and compound 5A

(50.5 mg, 351 μmol, 1.50 eq, HCl salt) in dioxane (2 mL) was added Cs₂CO₃ (229 mg, 703 μmol, 3.00 eq), Pd₂(dba)₃ (21.5 mg, 23.4 μmol, 0.10 eq) and XPhos (22.3 mg, 46.9 μmol, 0.20 eq) in one portion under N₂. The mixture was stirred at 90° C. for 4 hours under N₂. The reaction mixture was filtered and concentrated under reduced pressure to give a residue. The residue was purified by reversed-phase HPLC (0.1% HCl in acetonitrile) to give compound 16-4 (60 mg, crude) as brown oil, which was used in the next step directly without further purification.

LCMS: RT=0.921 min, m/z=583.3 (M+1)⁺.

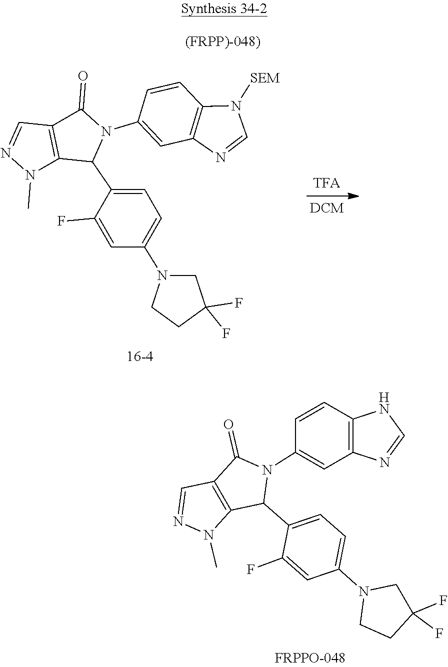 [SMILES: Cn1ncc2c1C(c1ccc(N3CCC(F)(F)C3)cc1F)N(c1ccc3c(c1)ncn3COCC[Si](C)(C)C)C2=O ; Cn1ncc2c1C(c1ccc(N3CCC(F)(F)C3)cc1F)N(c1ccc3[nH]cnc3c1)C2=O]

To a solution of compound 16-4 (50 mg, 85.8 μmol, 1.00 eq) in DCM (5 mL) was added TFA (3.08 g, 27.0 mmol, 2 mL, 315 eq). The mixture was stirred at 25° C. for 12 hrs. The reaction mixture was adjusted to pH=8 with Na₂CO₃ (aq), then extracted with DCM (20 mL×2). The combined organic layers were dried over Na₂SO₄, filtered and concentrated under reduced pressure to give a residue. The residue was purified by prep-HPLC (column: Waters Xbridge 150×25 mm, 5 μm; mobile phase: (10 mM NH₄HCO₃) A—water, B—ACN; gradient 16%-46% B, 10 min) to give FRPPO-048 (10.21 mg, 22.09 μmol, 25.8% yield, 97.9% purity) as a light yellow solid.

LCMS: RT=0.901 min, m/z=453.3 (M+1)⁺.

HPLC: RT=1.692 min.

¹H NMR: 400 MHz CDCl₃ δ 7.89 (s, 1H), 7.78 (s, 1H), 7.64-7.63 (m, 1H), 7.53-7.52 (m, 1H), 7.23-7.19 (m, 1H), 6.89-6.84 (m, 1H), 6.33-6.20 (m, 1H), 6.19-6.12 (m, 2H), 3.68 (s, 3H), 3.61-3.54 (m, 2H), 3.46-3.42 (m, 2H), 3.50-3.40 (m, 2H).

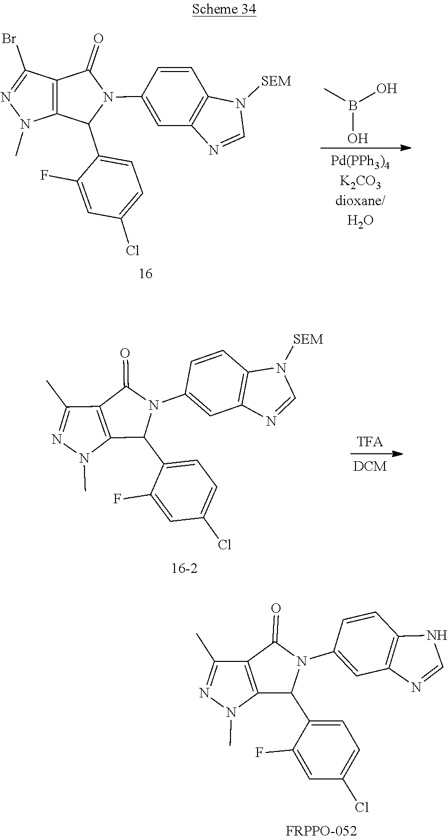

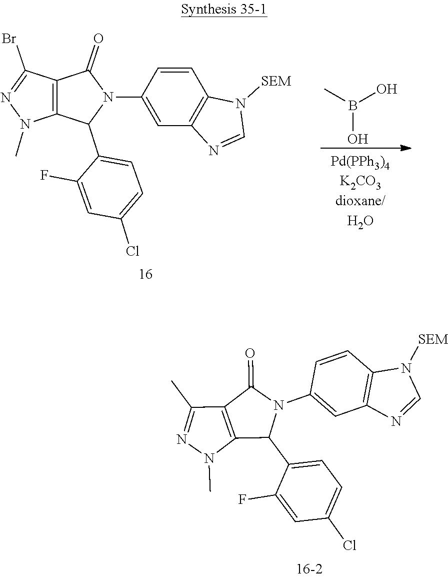

To a solution of compound 16 (200 mg, 338 μmol, 1.00 eq) and methylboronic acid (80 mg, 1.34 mmol, 3.95 eq) in dioxane (2 mL) and H₂O (0.5 mL) was added K₂CO₃ (100 mg, 724 μmol, 2.14 eq) and Pd(PPh₃)₄ (40 mg, 34.6 μmol, 0.1 eq). The mixture was stirred at 120° C. for 1 hr. The mixture was concentrated under reduced pressure to give a residue. The crude product was purified by reversed-phase HPLC (0.1% formic acid H₂O/MeCN) to give compound 16-2 (80 mg, 152 μmol, 44.9% yield, purity: 99%) as a yellow solid and used in the next step directly.

LCMS: RT=0.909 min, m/z=526.1 (M+1)⁺.

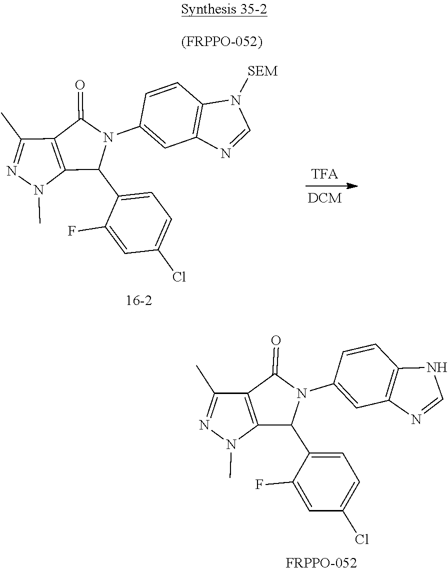 [SMILES: Cc1nn(C)c2c1C(=O)N(c1ccc3c(c1)ncn3COCC[Si](C)(C)C)C2c1ccc(Cl)cc1F ; Cc1nn(C)c2c1C(=O)N(c1ccc3[nH]cnc3c1)C2c1ccc(Cl)cc1F]

To a solution of compound 16-2 (30 mg, 57.0 μmol, 1.00 eq) in DCM (5 mL) was added TFA (13.0 mg, 114 μmol, 8.44 uL, 2.00 eq). The mixture was stirred at 25° C. for 12 hrs. The mixture was adjusted to pH 8 with Na₂CO₃ solution and extracted with DCM (10 mL×2). The combined organic layers were dried over anhydrous sodium sulfate, filtrated and concentrated to give a residue. The residue was purified by prep-HPLC (basic condition; column: Waters Xbridge 150×25 mm, 5 μm; mobile phase: (0.05% NH₄OH) A—water, B—ACN; gradient 22%-44% B, 10 min) to give FRPPO-052 (10.91 mg, 27.5 μmol, 48.2% yield, 99.8% purity) as a white solid.

LCMS: RT=0.843 min, m/z=396.2 (M+1)⁺.

HPLC: RT=1.585 min.

¹H NMR: 400 MHz CDCl₃ δ 9.47-9.37 (m, 1H), 7.99-7.96 (m, 1H), 7.75-7.69 (m, 1H), 7.63-7.45 (m, 1H), 7.43-7.27 (m, 1H), 7.10-7.04 (m, 3H), 6.39-6.37 (m, 1H), 3.62 (s, 3H), 2.49 (s, 3H).

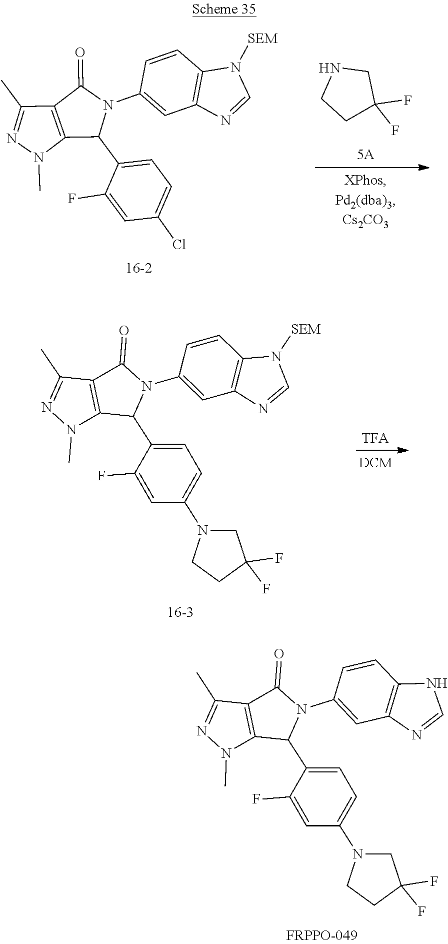 [SMILES: Cc1nn(C)c2c1C(=O)N(c1ccc3c(c1)ncn3COCC[Si](C)(C)C)C2c1ccc(Cl)cc1F ; FC1(F)CCNC1 ; Cc1nn(C)c2c1C(=O)N(c1ccc3c(c1)ncn3COCC[Si](C)(C)C)C2c1ccc(N2CCC(F)(F)C2)cc1F ; Cc1nn(C)c2c1C(=O)N(c1ccc3[nH]cnc3c1)C2c1ccc(N2CCC(F)(F)C2)cc1F]

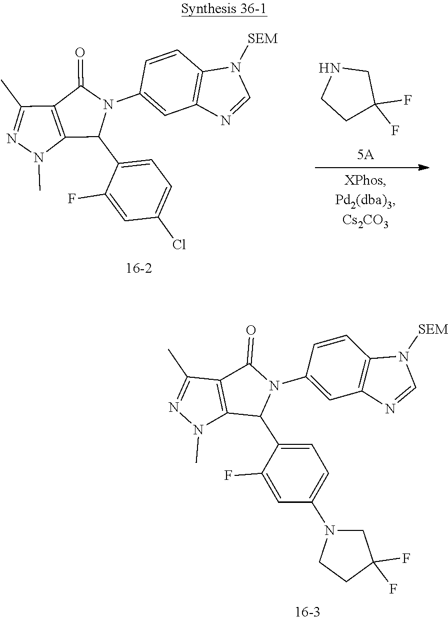 [SMILES: Cc1nn(C)c2c1C(=O)N(c1ccc3c(c1)ncn3COCC[Si](C)(C)C)C2c1ccc(Cl)cc1F ; FC1(F)CCNC1 ; Cc1nn(C)c2c1C(=O)N(c1ccc3c(c1)ncn3COCC[Si](C)(C)C)C2c1ccc(N2CCC(F)(F)C2)cc1F]

To a mixture of compound 16-2 (50 mg, 95.0 μmol, 164 uL, 1.00 eq) and compound 5A (20.5 mg, 143 μmol, 1.50 eq, HCl salt) in dioxane (2 mL) was added Cs₂CO₃ (92.9 mg, 285 μmol, 3.00 eq), Pd₂(dba)₃ (8.70 mg, 9.50 μmol, 0.1 eq) and XPhos (9.06 mg, 19.0 μmol, 0.20 eq) in one portion under N₂. The mixture was stirred at 90° C. for 3 hours under N₂. The reaction mixture was filtered and concentrated under reduced pressure to give a residue, which was purified by reversed-phase HPLC (0.1% FA in acetonitrile) to give compound 16-3 (30 mg, crude) as a yellow solid, which was used in the next step directly without further purification.

LCMS: RT=0.931 min, m/z=597.2 (M+1)⁺.

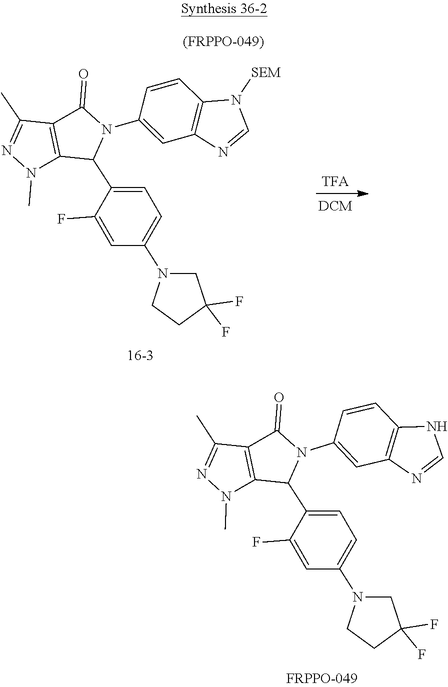 [SMILES: Cc1nn(C)c2c1C(=O)N(c1ccc3c(c1)ncn3COCC[Si](C)(C)C)C2c1ccc(N2CCC(F)(F)C2)cc1F ; Cc1nn(C)c2c1C(=O)N(c1ccc3[nH]cnc3c1)C2c1ccc(N2CCC(F)(F)C2)cc1F]

To a solution of 271-1 (20 mg, 33.5 μmol, 1.00 eq) in DCM (2 mL) was added TFA (3.08 g, 27.0 mmol, 2.00 mL). The mixture was stirred at 25° C. for 3 hrs. The reaction mixture was adjusted to pH=8 with Na₂CO₃ (aq), then extracted with DCM (20 mL×2). The combined organic layers were dried over Na₂SO₄, filtered and concentrated under reduced pressure to give a residue. The residue was purified by prep-HPLC (column: Xtimate C18 250×80 mm, 10 μm; mobile phase: (0.05% NH₄OH) A—water, B—ACN; gradient 28%-48% B, 10 min) to give FRPPO-049 (6.27 mg, 13.2 μmol, 39.4% yield, 98.2% purity) as a white solid.

LCMS: RT=0.880 min, m/z=467.3 (M+1)⁺.

HPLC: RT=1.985 min.

¹H NMR: 400 MHz CDCl₃ δ 7.89-7.88 (m, 1H), 7.66-7.27 (m, 2H), 7.09-7.01 (m, 1H), 7.01-6.87 (m, 1H), 6.28-6.19 (m, 1H), 6.16-6.12 (m, 2H), 3.60-3.54 (m, 5H), 3.46-3.42 (m, 2H), 2.49-2.42 (m, 5H).

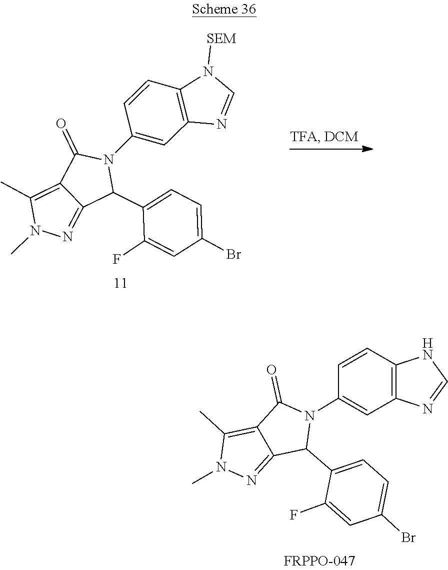 [SMILES: Cc1c2c(nn1C)C(c1ccc(Br)cc1F)N(c1ccc3c(c1)ncn3COCC[Si](C)(C)C)C2=O ; Cc1c2c(nn1C)C(c1ccc(Br)cc1F)N(c1ccc3[nH]cnc3c1)C2=O]

### Synthesis 37-1

### FRPPO-047

To a solution of compound 11 (50.0 mg, 87.6 μmol, 1.00 eq) in DCM (3 mL) was added TFA (3.08 g, 27.0 mmol, 2 mL, 308 eq). The mixture was stirred at 25° C. for 12 hrs. The reaction mixture was adjusted to pH=8 with Na₂CO₃ (aq), then extracted with DCM (20 mL×2). The combined organic layers were dried over Na₂SO₄, filtered and concentrated under reduced pressure to give a residue. The residue was purified by prep-HPLC (column: Xtimate C18 250×80 mm; 10 μm mobile phase: (0.05% NH₄OH) A—water, B—ACN; gradient 25%-45% B, 10 min) to give FRPPO-047 (20.67 mg, 46.9 μmol, 53.6% yield, 100% purity) as an off-white solid.

LCMS: RT=2.019 min, m/z=440.2 (M+1)⁺.

¹H NMR: 400 MHz CDCl₃ δ 7.99 (s, 1H), 7.70-7.69 (m, 1H), 7.55-7.53 (m, 1H), 7.25-7.20 (m, 3H), 6.99-6.98 (m, 1H), 6.37 (s, 1H), 3.62 (s, 3H), 2.49 (s, 3H).

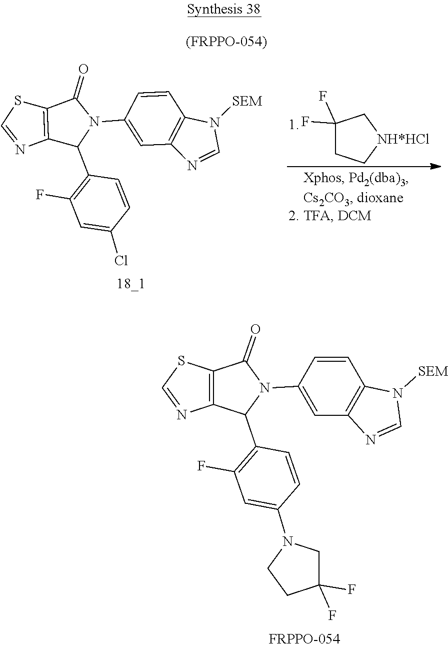

To a mixture of 18_1 (Scheme 28 and Synthesis 29-9) (100 mg, 194.15 μmol, 1 eq) and 3,3-difluoropyrrolidine (41.81 mg, 291.22 μmol, 1.5 eq, HCl) in dioxane (2 mL) was added Cs₂CO₃ (189.77 mg, 582.44 μmol, 3 eq), X-Phos (18.51 mg, 38.83 μmol, 0.2 eq) and Pd₂(dba)₃ (17.78 mg, 19.41 μmol, 0.1 eq) in one portion under N₂. The mixture was stirred at 90° C. for 4 hrs under N₂. The reaction mixture was filtered and concentrated under reduced pressure to give a residue. The residue was purified by reversed-phase HPLC (0.1% FA in acetonitrile) to give a brown solid (50 mg, crude) which was dissolved in DCM (3 mL) and treated with TFA (1.54 g, 13.51 mmol, 1 mL, 158.21 eq). The mixture was stirred at 25° C. for 12 hr. The reaction mixture was adjusted to pH 8 with Na₂CO₃ (aq), then extracted with DCM (20 mL*2). The combined organic layers were dried over Na₂SO₄, filtered and concentrated under reduced pressure to give a residue, which was purified by prep-HPLC (base condition; column: Waters Xbridge 150*25 mm*5 μm; mobile phase: (0.05% NH₄OH) A—water, B—ACN; gradient 30%-50% B, 10 min) to give FRPPO-054 (3.89 mg, 8.54 μmol, 10.01% yield, 100% purity) as a yellow solid.

LCMS: RT=0.894 min, m/z=456.3 (M+1)⁺.

HPLC: RT=1.807 min.

¹H NMR: 400 MHz CDCl₃ δ 9.09 (s, 1H), 8.01 (s, 1H), 7.80 (s, 1H), 7.63-7.35 (m, 2H), 6.82-6.83 (m, 1H), 6.46 (s, 1H), 6.16-6.14 (m, 2H), 3.58-3.52 (m, 2H), 3.43-3.40 (m, 2H), 2.49-2.38 (m, 2H).

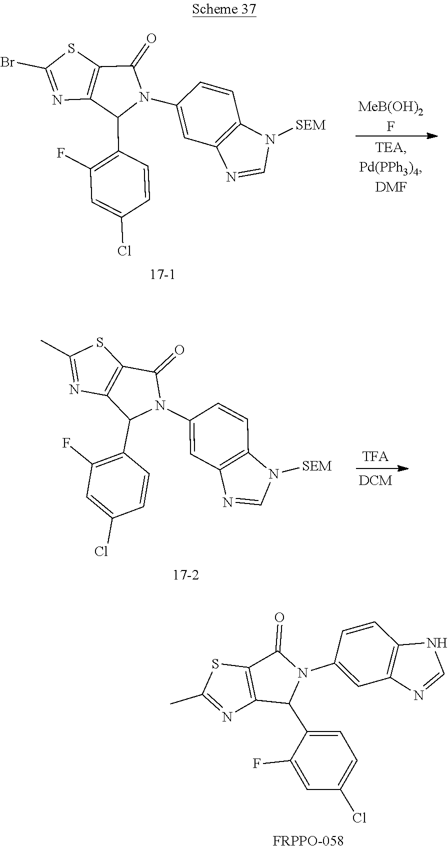 [SMILES: "MeB(OH)2F" ; C[Si](C)(C)CCOCn1cnc2cc(N3C(=O)c4sc(Br)nc4C3c3ccc(Cl)cc3F)ccc21 ; Cc1nc2c(s1)C(=O)N(c1ccc3c(c1)ncn3COCC[Si](C)(C)C)C2c1ccc(Cl)cc1F ; Cc1nc2c(s1)C(=O)N(c1ccc3[nH]cnc3c1)C2c1ccc(Cl)cc1F]

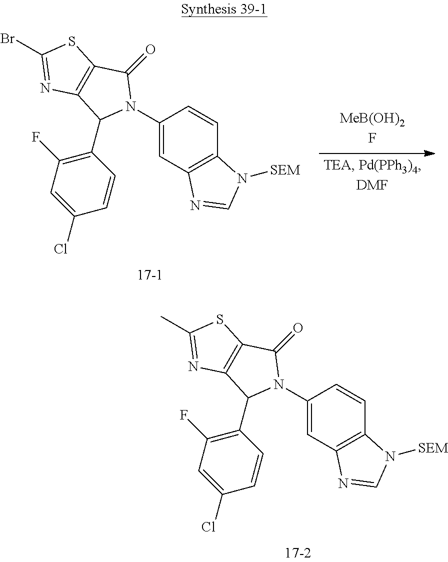 [SMILES: C[Si](C)(C)CCOCn1cnc2cc(N3C(=O)c4sc(Br)nc4C3c3ccc(Cl)cc3F)ccc21 ; Cc1nc2c(s1)C(=O)N(c1ccc3c(c1)ncn3COCC[Si](C)(C)C)C2c1ccc(Cl)cc1F]

To a mixture of compound 17-1 (Synthesis 29-8) (100 mg, 135 μmol, 1.00 eq), TEA (50.0 mg, 494 μmol, 68.8 μL, 3.67 eq) and compound F (25.0 mg, 418 μmol, 3.10 eq) in DMF (2 mL) was added Pd(PPh₃)₄ (25.0 mg, 21.6 μmol, 0.16 eq) in one portion at 20° C. under N₂. The mixture was stirred at 100° C. for 12 hrs. The mixture was filtered and concentrated in vacuum. The residue was purified by reversed-phase HPLC (0.1% FA condition in acetonitrile) to give compound 17-2 (20 mg crude) as a yellow solid which was used directly in subsequent reactions.

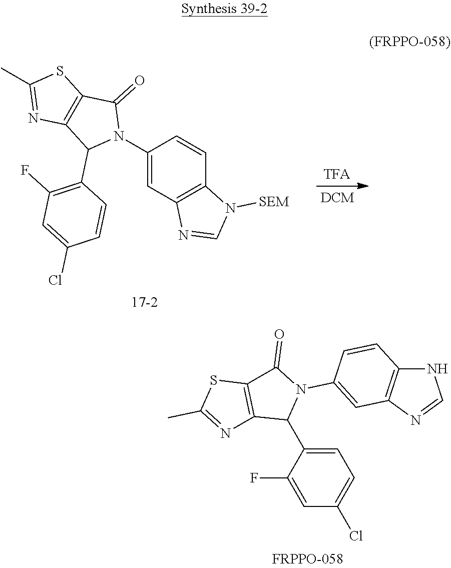 [SMILES: Cc1nc2c(s1)C(=O)N(c1ccc3c(c1)ncn3COCC[Si](C)(C)C)C2c1ccc(Cl)cc1F ; Cc1nc2c(s1)C(=O)N(c1ccc3[nH]cnc3c1)C2c1ccc(Cl)cc1F]

To a solution of compound 17-2 (10.0 mg, 18.9 μmol, 1.00 eq) in DCM (5 mL) was added TFA (1.54 g, 13.5 mmol, 1 mL, 715 eq). The mixture was stirred at 25° C. for 10 hrs. The reaction mixture was adjusted to pH=8 with Na₂CO₃ (aq), then extracted with DCM (20 mL*2). The combined organic layers were dried over Na₂SO₄, filtered and concentrated under reduced pressure to give a residue. The residue was purified by prep-HPLC (basic condition, column: Xtimate C18 150*40 mm*10 μm; mobile phase: [water (0.05% ammonia hydroxide v/v)-ACN]; B %: 23%-53%, 10 min) to give FRPPO-058 (2.33 mg, 5.71 μmol, 30.2% yield, 97.7% purity) as a yellow solid.

LCMS: RT=0.967 min, m/z=521.2 (M+1)⁺.

HPLC: RT=2.105 min.

¹H NMR: 400 MHz CDCl₃ δ 8.03 (s, 1H), 7.78 (s, 1H), 7.45-7.41 (m, 1H), 7.08-7.00 (m, 4H), 6.45 (s, 1H), 2.83 (s, 3H).

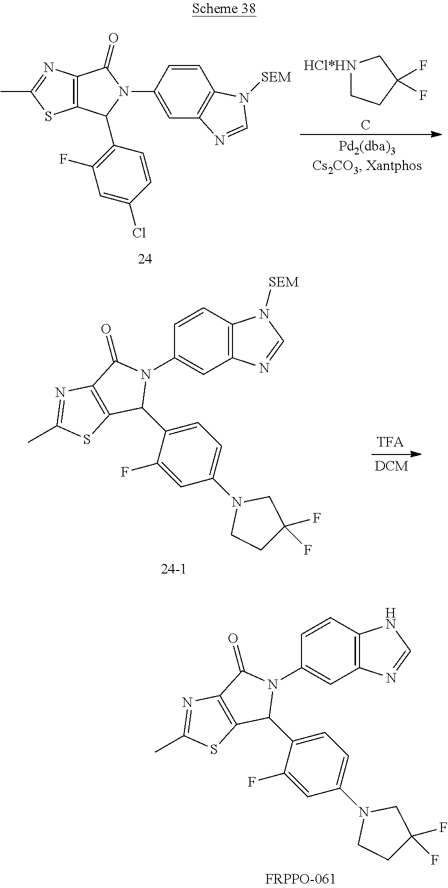

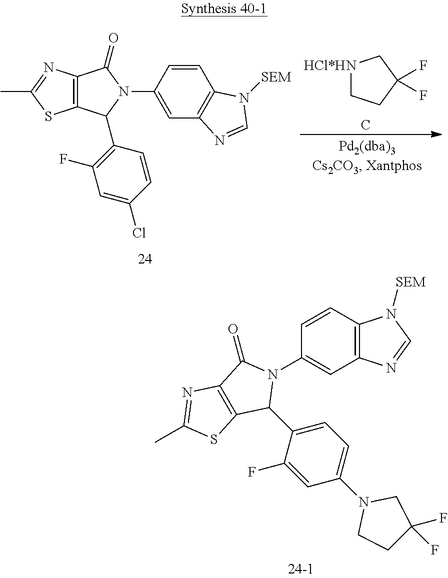

To a solution of compound 24 (Scheme 24 and Synthesis 25-6) (120 mg, 226 μmol, 1.00 eq) and compound C (48.4 mg, 340 μmol, 1.50 eq, HCl) in dioxane (3 mL) was added Cs₂CO₃ (221 mg, 680 μmol, 3.00 eq) and Xantphos (21.6 mg, 45.3 μmol, 0.20 eq) and Pd₂(dba)₃ (20.7 mg, 22.6 μmol, 0.10 eq), the mixture was stirred at 90° C. for 3 hrs. The mixture was diluted with water 20 mL and extracted with DCM 30 mL (10 mL*3). The combined organic layers were washed with brine 20 mL, dried over Na₂SO₄, filtered and concentrated under reduced pressure to give compound 24-1 (48 mg, crude) as a yellow solid, which was used in the next directly.

LCMS: RT=0.900 min, m/z=600.2 (M+1)⁺.

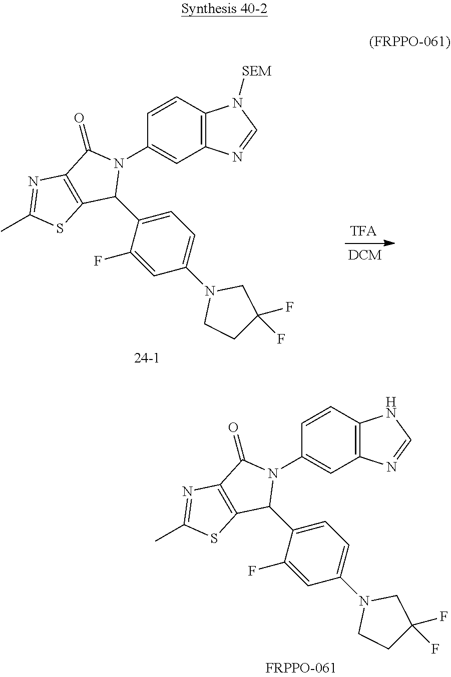 [SMILES: Cc1nc2c(s1)C(c1ccc(N3CCC(F)(F)C3)cc1F)N(c1ccc3c(c1)ncn3COCC[Si](C)(C)C)C2=O ; Cc1nc2c(s1)C(c1ccc(N3CCC(F)(F)C3)cc1F)N(c1ccc3[nH]cnc3c1)C2=O]

To a solution of compound 24-1 (80 mg, 133 μmol, 1.00 eq) in DCM (1 mL) was added TFA (1.54 g, 13.5 mmol, 1 mL, 101 eq). The mixture was stirred at 25° C. for 2 hrs. The reaction mixture was adjusted to pH=8-9 with NH₃H₂O. The mixture was diluted with water 20 mL and extracted with DCM 30 mL (10 mL*3). The combined organic layers were washed with brine 20 mL, dried over Na₂SO₄, filtered and concentrated under reduced pressure to give a residue. The residue was purified by prep-HPLC (Xtimate C18 150*40 mm*10 μm; mobile phase: (0.05% NH₄OH) A—water, B—ACN; gradient 30%-60% B, 10 min) to give FRPPO-061 (1.79 mg, 3.68 μmol, 2.76% yield, 96.6% purity) as a white solid.

LCMS: RT=0.821 min, m/z=469.9 (M+1)⁺.

HPLC: RT=2.142 min.

¹H NMR: 400 MHz DMSO-d6 δ 8.66 (s, 1H), 7.80 (s, 1H), 7.64-7.61 (m, 1H), 7.46-7.44 (m, 1H), 7.31-7.29 (m, 1H), 7.11-7.05 (m, 1H), 7.03-6.83 (m, 1H), 6.32-6.25 (m, 2H), 3.62-3.56 (m, 2H), 2.79 (s, 3H), 2.67-2.66 (m, 1H), 2.55-2.43 (m, 1H), 2.43-2.43 (m, 1H), 2.33-2.32 (m, 1H).

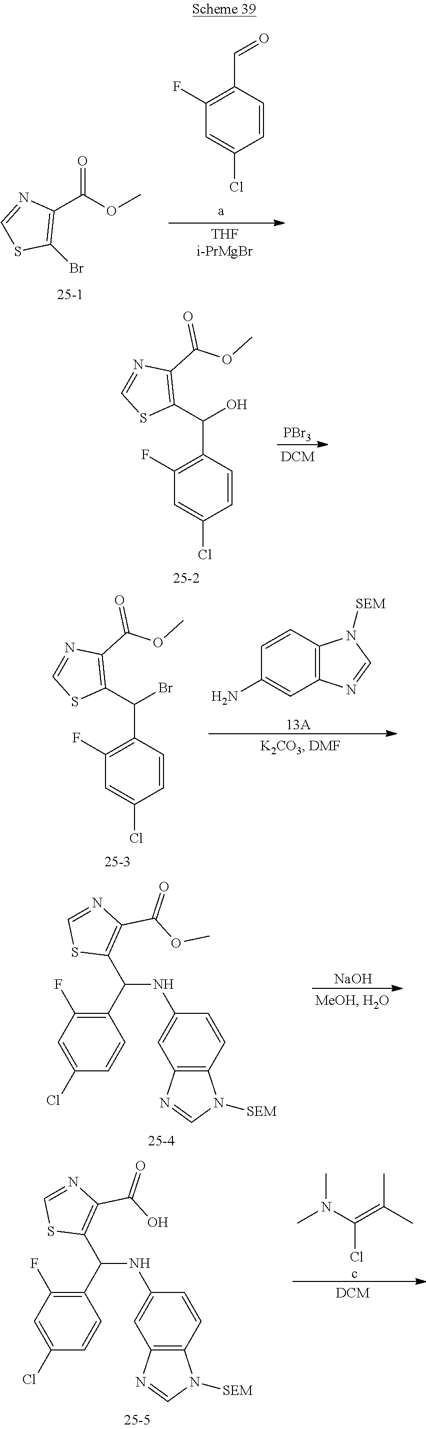

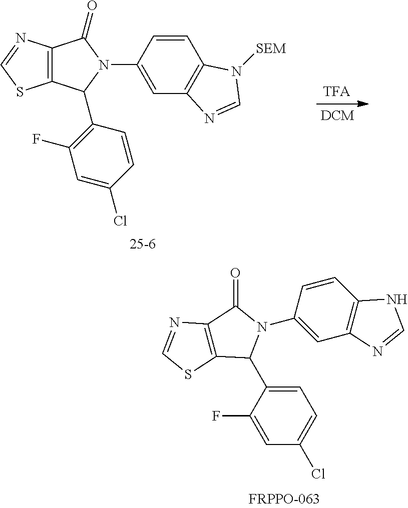 [SMILES: C[Si](C)(C)CCOCn1cnc2cc(N3C(=O)c4ncsc4C3c3ccc(Cl)cc3F)ccc21 ; O=C1c2ncsc2C(c2ccc(Cl)cc2F)N1c1ccc2[nH]cnc2c1]

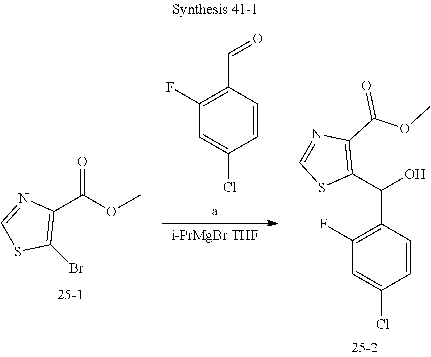

To a solution of compound 25-1 (2.50 g, 10.6 mmol, 1.00 eq) in THF (20 mL) was added i-PrMgBr (1 M, 21.2 mL, 2.00 eq) at −70° C., the reaction was stirred at −70° C. for 10 min, compound a (2.52 g, 15.9 mmol, 1.50 eq) was added, the resulting suspension was stirred at −40° C. for 1 hr. TLC (PE/EA=2/1) indicated compound 25-1 (R_{f}=0.6) was consumed completely and one new spot (R_{f}=0.2) formed. The reaction was (clean) according to TLC. The reaction was added to NH₄Cl (100 mL) and extracted with ethyl acetate (50 mL*2). The combined organic phase was washed with brine (50 mL), dried with anhydrous Na₂SO₄, filtered and concentrated in vacuum. The residue was purified by column chromatography (SiO₂, petroleum ether:ethyl acetate=1:1 to 0:1) to give compound 25-2 (1.68 g, 5.32 mmol, 50.3% yield) as colorless oil.

¹H NMR: 400 MHz CDCl₃ δ 8.67 (s, 1H), 7.61-7.57 (m, 1H), 7.23-7.21 (m, 1H), 7.13-7.10 (m, 1H), 6.70-6.69 (d, J=4.4 Hz, 1H), 4.76-4.75 (d, J=4.4 Hz, 1H), 4.51-4.46 (m, 2H), 1.31-1.17 (m, 3H).

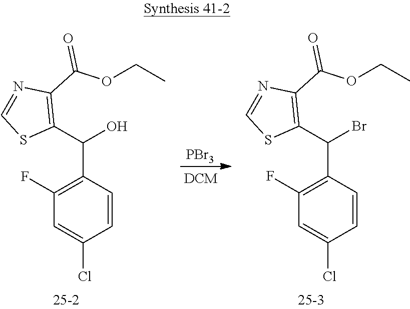 [SMILES: CCOC(=O)c1ncsc1C(O)c1ccc(Cl)cc1F ; "PBr3" ; CCOC(=O)c1ncsc1C(Br)c1ccc(Cl)cc1F]

To a solution of compound 25-2 (1.38 g, 4.37 mmol, eq in DCM (50 m was added dropwise a solution of PBr₃ (5.92 g, 21.9 mmol, 5.00 eq) in DCM (5 mL) at 0° C. The reaction was stirred at 0° C. for 1 hr. The reaction was pour into ice-water (50 mL), then the reaction mixture was extracted with DCM (50 mL*2). The combined organic phase was washed with brine (50 mL), dried with anhydrous Na₂SO₄, filtered and concentrated in vacuum to give compound 25-3 (1.50 g, crude) as yellow oil, which was used in the next step directly.

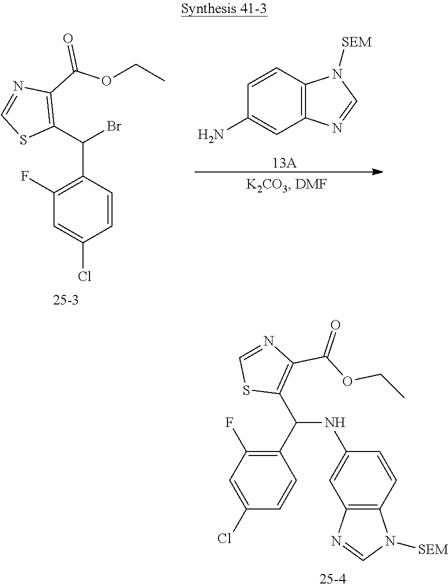

To a solution of compound 25-3 (1.50 g, 3.96 mmol, 1.00 eq) in DMF (50 mL) was added K₂CO₃ (1.10 g, 7.92 mmol, 2.00 eq); compound 13A (Scheme 68 and Synthesis 79-2) at 20° C., the reaction was stirred at 80° C. for 2 hrs. To this reaction was added H₂O (50 mL), then the mixture was extracted ethyl acetate (50 mL*2). The combined organic phase was washed with brine (50 mL), dried with anhydrous Na₂SO₄, filtered and concentrated in vacuum. The residue was purified by prep-TLC (SiO₂, petroleum ether:ethyl acetate=1:1) to give compound 25-4 (0.100 g, 178.21 μmol, 4.50% yield) as a yellow oil.

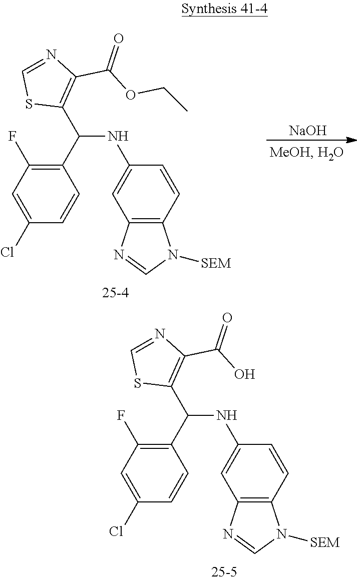 [SMILES: CCOC(=O)c1ncsc1C(Nc1ccc2c(c1)ncn2COCC[Si](C)(C)C)c1ccc(Cl)cc1F ; "NaOH" ; C[Si](C)(C)CCOCn1cnc2cc(NC(c3ccc(Cl)cc3F)c3scnc3C(=O)O)ccc21]

To a solution of compound 25-4 (300 mg, 535 μmol, 1.00 eq) in H₂O (2 mL) and MeOH (6 mL) was added NaOH (110 mg, 2.75 mmol, 5.14 eq). The mixture was stirred at 25° C. for 2 hrs. The mixture was concentrated under reduced pressure to give a residue. The residue was diluted in water (20 mL) and adjusted to pH=5 with HCl solution (1 M). The mixture was extracted with EtOAc (30 mL*2). The combined organic layers were dried over anhydrous sodium sulfate, filtrated and concentrated to give compound 25-5 (280 mg, 525 μmol, 98.2% yield) as a black brown solid.

LCMS: RT=0.881 min. m/z=533.1 (M+1)⁺.

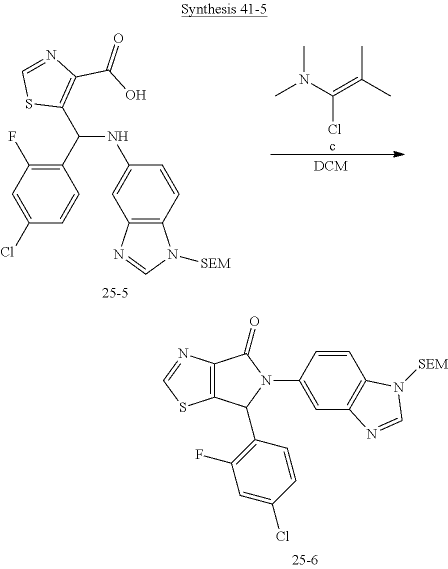

To a solution of compound 25-5 (280 mg, 525 μmol, 1.00 eq) in DCM (6 mL) was added compound 5a (230 mg, 1.72 mmol, 228 μL, 3.28 eq) at 0° C. The mixture was stirred at 0° C. for 2 hrs. The reaction mixture was concentrated under reduced pressure to give a residue, which was purified by reversed-phase HPLC (0.1% FA condition in acetonitrile) to give compound 25-6 (120 mg, 233 μmol, 44.4% yield) as a yellow oil.

LCMS: RT=0.893 min, m/z=515.1 (M+1)⁺.

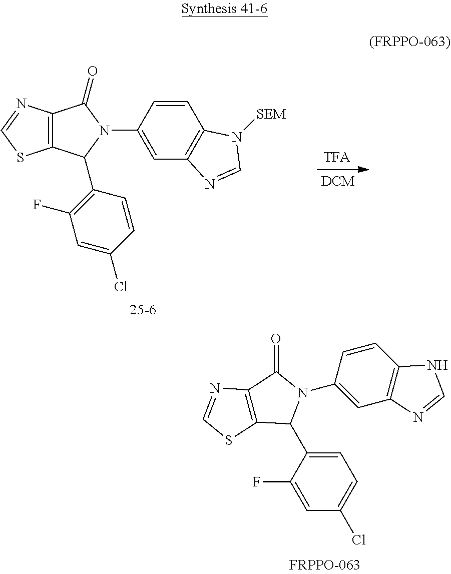 [SMILES: C[Si](C)(C)CCOCn1cnc2cc(N3C(=O)c4ncsc4C3c3ccc(Cl)cc3F)ccc21 ; O=C1c2ncsc2C(c2ccc(Cl)cc2F)N1c1ccc2[nH]cnc2c1]

To a solution of compound 25-6 (20 mg, 38.8 μmol, 1.00 eq) in DCM (4 mL) was added TFA (1.54 g, 13.5 mmol, 1 mL, 348 eq). The mixture was stirred at 25° C. for 12 hrs. The reaction mixture was adjusted pH-7 with NH₃H₂O and concentrated under reduced pressure to give a residue. The residue was purified by prep-HPLC (Xtimate C18 150*40 mm*10 μm; mobile phase: (0.05% NH₄OH) A—water, B—ACN; gradient 18%-48% B, 10 min) to give FRPPO-063 (2.67 mg, 6.52 μmol, 16.8% yield, 93.9% purity) as a white solid.

LCMS: RT=0.860 min, m/z=385.1 (M+1)⁺.

HPLC: RT=1.645 min.

¹H NMR: 400 MHz DMSO-d6 δ 12.5 (s, 1H), 9.36 (s, 1H), 8.21 (s, 1H), 7.76-7.72 (m, 1H), 7.41-7.38 (m, 1H), 7.33-7.31 (m, 3H), 7.19-7.16 (m, 1H), 7.00 (s, 1H).

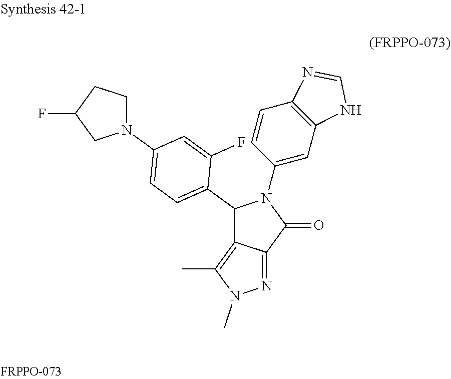 [SMILES: Cc1c2c(nn1C)C(=O)N(c1ccc3nc[nH]c3c1)C2c1ccc(N2CCC(F)C2)cc1F]

The procedures for preparation of FRPPO-073 was similar to FRPPO-072 (Scheme 21 and Synthesis 22-1). FRPPO-073 (8.66 mg, 17.8 μmol, 10.3% yield, 92.1% purity) was obtained as off-white solid.

LCMS: RT=0.925 min, m/z=449.3 [M+1]+.

HPLC: RT=2.747 min, 92.1% purity.

¹H NMR: 400 MHz DMSO-d₆ δ 12.34-12.32 (m, 1H), 9.90-9.82 (m, 1H), 8.19-8.16 (m, 1H), 7.57-7.43 (m, 1H), 7.42-7.40 (m, 1H), 7.07 (t, J=8.4 Hz, 1H), 6.31-6.29 (m, 3H), 5.49-5.36 (m, 1H), 3.85 (s, 3H), 3.44-3.36 (m, 4H), 2.30 (s, 3H), 2.23-2.08 (m, 2H).

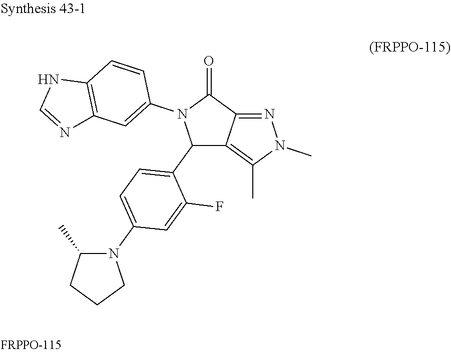 [SMILES: Cc1c2c(nn1C)C(=O)N(c1ccc3[nH]cnc3c1)C2c1ccc(N2CCC[C@@H]2C)cc1F]

The procedures for preparation of FRPPO-115 was similar to FRPPO-072 (Scheme 21 and Synthesis 22-1). FRPPO-115 (1.21 mg, 2.51 μmol, 4.81% yield, 92.3% purity) was obtained as a white solid.

LCMS: RT=0.902 min, m/z=445.2 (M+1)⁺.

HPLC: RT=2.290 min.

¹H NMR: 400 MHz DMSO-d6 δ 8.17 (s, 1H), 7.65 (s, 1H), 7.05-7.46 (m, 1H), 7.30-7.28 (m, 1H), 6.93-6.89 (m, 1H), 6.40-6.39 (m, 1H), 6.20-6.11 (m, 2H), 3.85 (m, 4H), 3.06-2.85 (m, 2H), 2.14 (s, 3H), 1.91-1.87 (m, 2H), 1.56 (s, 2H), 0.99-0.96 (m, 3H).

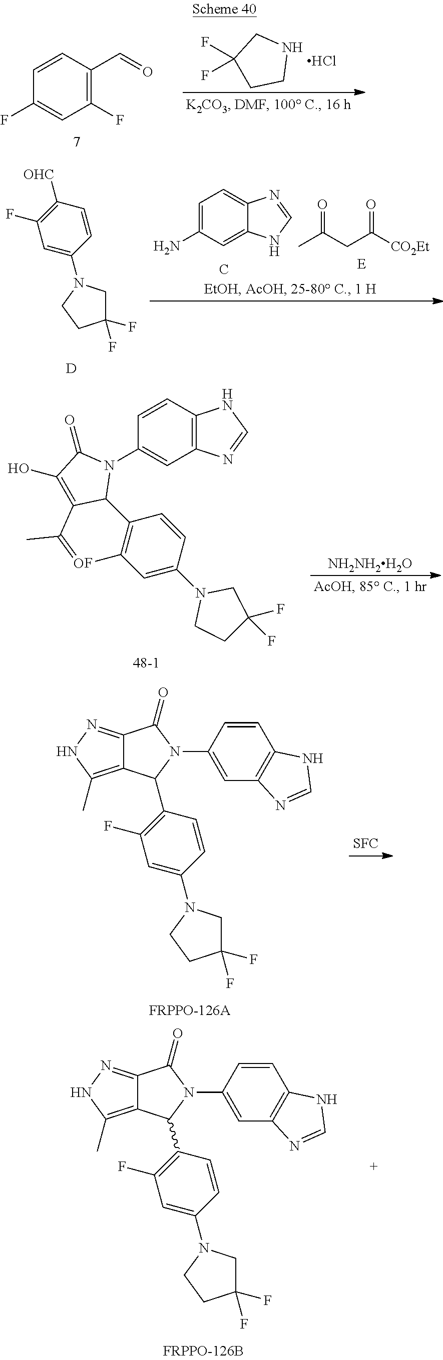

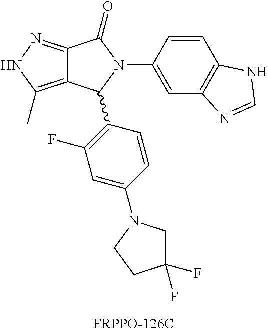 [SMILES: Cc1[nH]nc2c1C(c1ccc(N3CCC(F)(F)C3)cc1F)N(c1ccc3[nH]cnc3c1)C2=O]

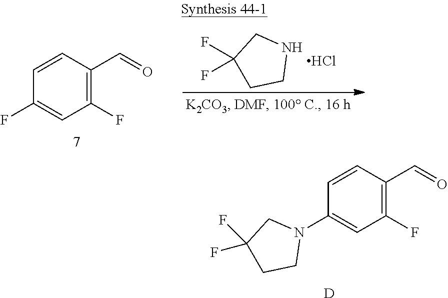

To a mixture of 3,3-difluoropyrrolidine (12.12 g, 84.45 mmol, 1.2 eq, HCl) and K₂CO₃ (14.59 g, 105.56 mmol, 1.5 eq) in DMF (50 mL) was added 2,4-difluorobenzaldehyde 7 (10 g, 70.37 mmol, 1 eq). The mixture was stirred at 100° C. for 16 hrs. TLC (petroleum ether:ethyl acetate=5:1) indicated trace of 2,4-difluorobenzaldehyde was remained, and some new spots with was detected. The reaction mixture was diluted with H₂O 100 (mL) and extracted with Ethyl acetate (50 mL*3). The combined organic layers were washed with brine (50 mL), dried over anhydrous Na₂SO₄, filtered and concentrated under reduced pressure to give a residue. The residue was purified by column chromatography (SiO₂, petroleum ether:ethyl acetate=1:0 to 5:1). Compound D (9.3 g, 34.64 mmol, 49.22% yield, 85% purity) was obtained as yellow solid.

LCMS: RT=0.779 min, m/z=230.2 (M+1)⁺.

¹H NMR (400 MHz, CDCl₃) δ 10.11 (s, 1H), 7.76 (t, J=8.4 Hz, 1H), 6.36 (dd, J=2.1, 8.7 Hz, 1H), 6.18 (dd, J=2.3, 13.4 Hz, 1H), 3.75 (t, J=12.7 Hz, 2H), 3.63 (t, J=7.3 Hz, 2H), 2.62-2.48 (m, 2H).

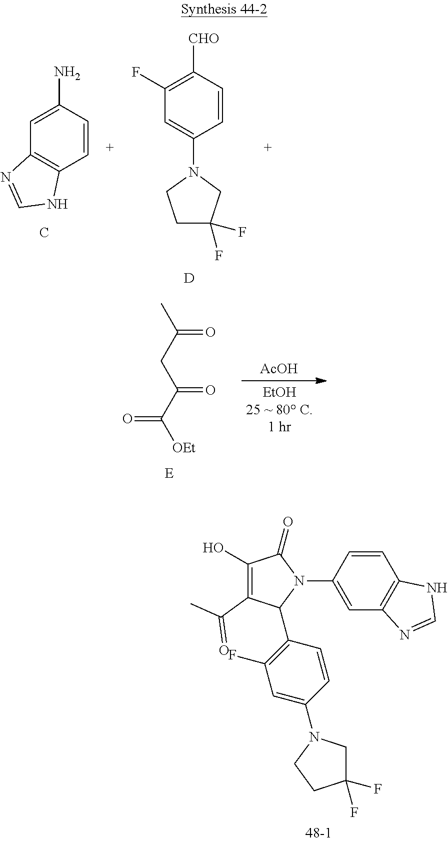

To a solution of compound D (1.00 g, 4.36 mmol, 1.00 eq) in EtOH (5 mL) was added compound C (700 mg, 5.26 mmol, 1.20 eq) and AcOH (263 mg, 4.37 mmol, 250 μL, 1.00 eq). The mixture was stirred at 25° C. for 0.5 hr. Then compound E (690 mg, 4.36 mmol, 616 μL, 1.00 eq) was added, the mixture was stirred at 80° C. for 0.5 hr. The mixture was filtered and the cake was washed with EtOH (50 mL) to give compound 48-1 (0.90 g, 1.97 mmol, 45.2% yield) as a brown solid.

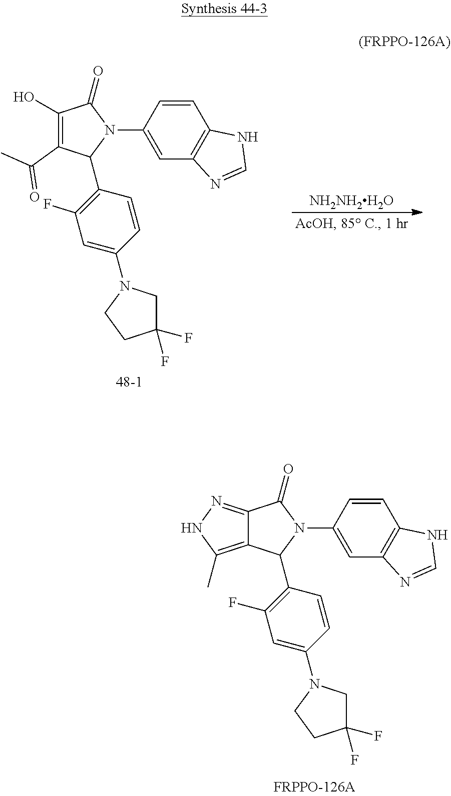 [SMILES: CC(=O)C1=C(O)C(=O)N(c2ccc3[nH]cnc3c2)C1c1ccc(N2CCC(F)(F)C2)cc1F ; "NH2NH2·H2O" ; Cc1[nH]nc2c1C(c1ccc(N3CCC(F)(F)C3)cc1F)N(c1ccc3[nH]cnc3c1)C2=O]

To a solution of 48-1 (900 mg, 1.97 mmol, 1.00 eq) in AcOH (5 mL) was added NH₂NH₂.H₂O (206 mg, 4.03 mmol, 200 μL, 2.05 eq). The mixture was stirred at 85° C. for 1 hr. NH₃.H₂O was added to adjust the pH to 8. The mixture was filtered and the residue was purified by prep-HPLC (neutral condition; lumn: Waters Xbridge 150*50 mm*10 μm; mobile phase: (10 mM NH₄HCO₃) A—water, B—ACN; gradient 20%-50% B, 10 min) to give FRPPO-126A (80 mg, 172 μmol, 8.72% yield, 97.2% purity) as a yellow solid.

¹H NMR: 400 MHz MeOD δ 8.15 (s, 1H), 7.61-7.53 (m, 2H), 7.31-7.28 (m, 1H), 6.95-6.93 (m, 1H), 6.39 (s, 1H), 6.29-6.19 (m, 2H), 3.59-3.52 (m, 2H), 3.42-3.39 (m, 2H), 2.46-2.38 (m, 2H), 2.18 (s, 3H).

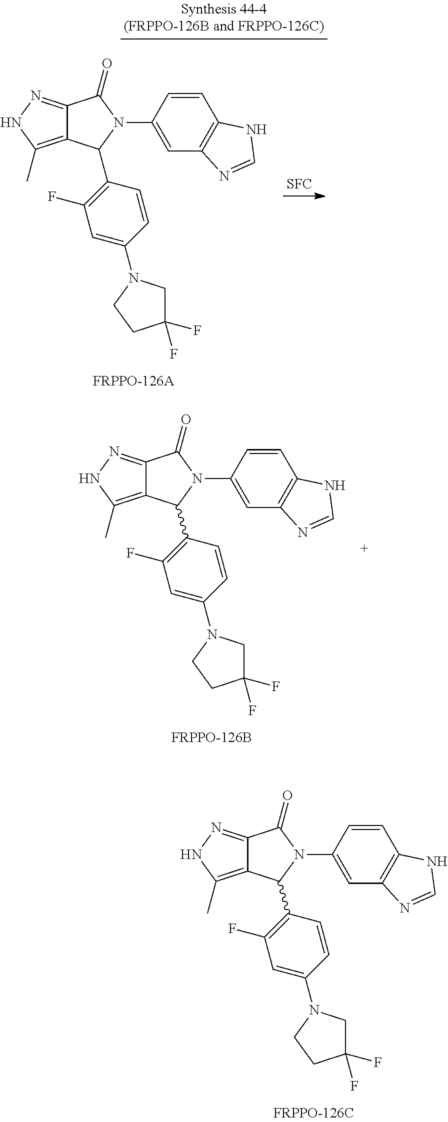

FRPPO-126A (80 mg, 177 μmol, 1.00 eq) was separated by chiral SFC chromatography. Column: Chiralpak AD-3 250×30 mm ID, 10 micron particle size. Mobile phase: 60% A—C02; 40% B—EtOH (0.05% DEA) at 70 g/min, 35° C., to give: FRPPO-126B (30.65 mg, 67.1 μmol, 86.7% yield, 99.0% purity) as a yellow solid.

LCMS: RT=0.838 min. m/z=453.2 (M+1)⁺.

HPLC: RT=2.007 min.

SFC: RT=1.89 min.

¹H NMR: 400 MHz DMSO-d6 δ 13.3 (s, 1H), 12.4 (s, 1H), 8.19 (s, 1H), 7.66 (s, 1H), 7.65-7.49 (m, 1H), 7.30-7.28 (m, 1H), 7.00-6.98 (m, 1H), 6.45 (s, 1H), 6.29-6.24 (m, 2H), 3.62-3.55 (m, 2H), 3.37 (s, 2H), 2.45-2.38 (m, 2H), 2.09 (s, 3H).

FRPPO-126C (41.36 mg, 88.7 μmol, 115% yield, 97.0% purity) as a yellow solid.

LCMS: RT=0.859 min, m/z=453.1 (M+1)⁺.

HPLC: RT=2.042 min.

SFC: RT=2.19 min.

¹H NMR: 400 MHz DMSO-d6 δ 13.3 (s, 1H), 12.5 (s, 1H), 8.18 (s, 1H), 7.65 (s, 1H), 7.51-7.48 (m, 1H), 7.30-7.28 (m, 1H), 7.00-6.98 (m, 1H), 6.45 (s, 1H), 6.30-6.24 (m, 2H), 3.65-3.52 (m, 2H), 3.37 (s, 2H), 2.45-2.38 (m, 2H), 2.09 (s, 3H).

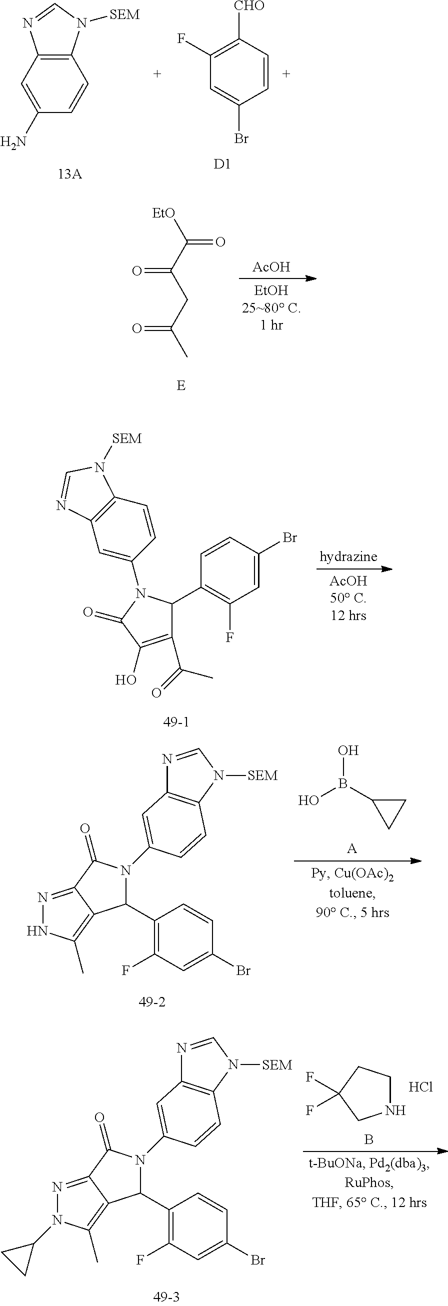

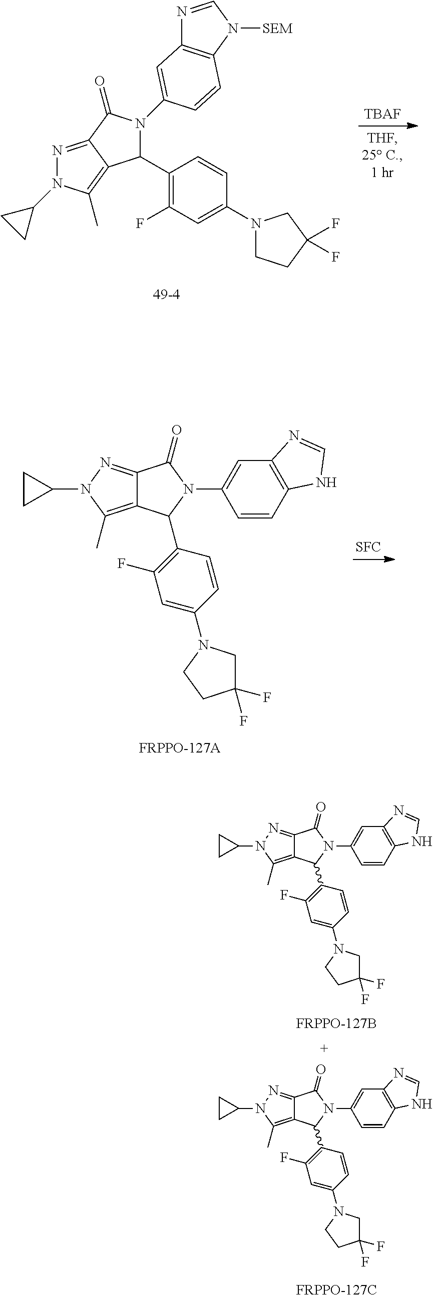 [SMILES: Cc1c2c(nn1C1CC1)C(=O)N(c1ccc3c(c1)ncn3COCC[Si](C)(C)C)C2c1ccc(N2CCC(F)(F)C2)cc1F ; "TBAF" ; Cc1c2c(nn1C1CC1)C(=O)N(c1ccc3[nH]cnc3c1)C2c1ccc(N2CCC(F)(F)C2)cc1F ; "SFC" ; Cc1c2c(nn1C1CC1)C(=O)N(c1ccc3[nH]cnc3c1)C2c1ccc(N2CCC(F)(F)C2)cc1F ; Cc1c2c(nn1C1CC1)C(=O)N(c1ccc3[nH]cnc3c1)C2c1ccc(N2CCC(F)(F)C2)cc1F]

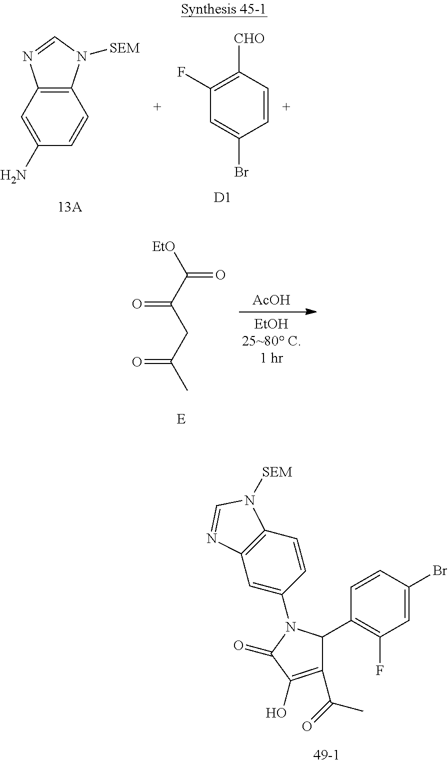

To a solution of compound D1 (7.71 g, 37.9 mmol, 1.00 eq) and compound 13A (Scheme 68 and Synthesis 79-2) (10.0 g, 37.9 mmol, 1.00 eq) in EtOH (100 mL) was added AcOH (2.39 g, 39.8 mmol, 2.28 mL, 1.05 eq). The mixture was stirred at 25° C. for 0.5 hr. And then compound E (6.00 g, 37.9 mmol, 5.36 mL, 1.00 eq) was added, the mixture was stirred at 80° C. for 0.5 hr. The reaction mixture was filtered and concentrated under reduced pressure to give compound 49-1 (15.0 g, 26.7 mmol, 70.5% yield) as a light yellow solid.

¹H NMR: 400 MHz DMSO-d6 δ 8.37-8.35 (m, 1H), 7.78 (s, 1H), 7.60-7.56 (m, 1H), 7.39-7.19 (m, 4H), 6.25 (s, 1H), 5.57-5.56 (m, 2H), 3.46-3.42 (m, 2H), 2.36 (s, 3H), 0.80-0.78 (m, 2H), −0.11-−0.17 (m, 9H).

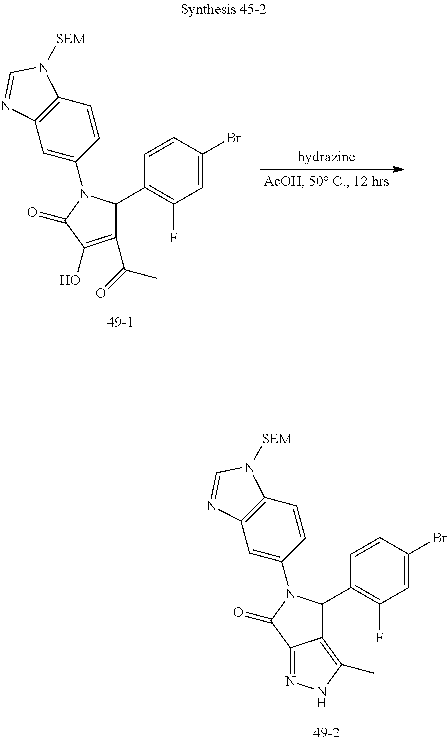 [SMILES: CC(=O)C1=C(O)C(=O)N(c2ccc3c(c2)ncn3COCC[Si](C)(C)C)C1c1ccc(Br)cc1F ; Cc1[nH]nc2c1C(c1ccc(Br)cc1F)N(c1ccc3c(c1)ncn3COCC[Si](C)(C)C)C2=O]

To a solution of compound 49-1 (8.00 g, 14.2 mmol, 1.00 eq) in AcOH (30 mL) was added hydrazine (1.44 g, 44.9 mmol, 1.63 mL, 3.15 eq). The mixture was stirred at 50° C. for 12 hrs. The reaction mixture was diluted with water 30 mL and extracted with EtOAc 150 mL (50 mL*3). The combined organic layers were washed with water 100 mL (50 mL*2) and saturated NaHCO₃ 100 mL (50 mL*2) concentrated under reduced pressure to give a residue to give 49-2 (9.00 g, crude) as a red solid.

¹H NMR: 400 MHz CDCl₃ δ 7.96 (d, J=1.6 Hz, 1H), 7.75 (d, J=1.7 Hz, 1H), 7.54-7.51 (m, 1H), 7.31-7.27 (m, 1H), 7.21-7.19 (m, 2H), 7.18-6.93 (m, 1H), 6.45-6.42 (m, 1H), 5.56-5.45 (m, 2H), 3.51-3.47 (m, 2H), 2.28 (s, 3H), 0.91-0.87 (m, 2H), 0.03-0.07 (m, 8H).

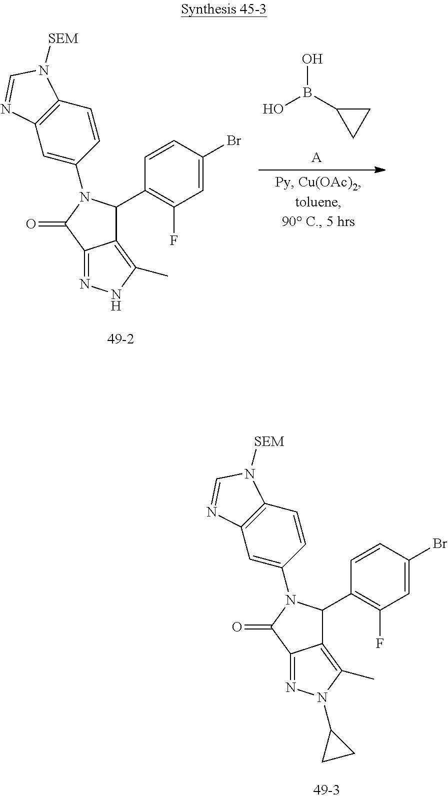

To a solution of compound 49-2 (0.500 g, 898 μmol, 1.00 eq) and compound A (100 mg, 1.17 mmol, 1.30 eq) in toluene (10 mL) was added Py (213 mg, 2.70 mmol, 217 μL, 3.00 eq) and Cu(OAc)₂ (244 mg, 1.35 mmol, 1.50 eq). The mixture was degassed and purged with O₂ for 3 times, and then the mixture was stirred at 90° C. for 5 hrs under O₂ atmosphere (15 Psi). The reaction mixture was filtered and concentrated under reduced pressure to give a residue. The crude product was purified by reversed-phase HPLC (0.1% NH₃.H₂O in acetonitrile) to give 49-3 (0.300 g, 502 μmol, 27.9% yield) as a yellow oil.

LCMS: EW19673-18-P1A, RT=1.118 min, m/z=596.1, 598.3 (M+1)⁺.

¹H NMR: 400 MHz CDCl₃ δ 8.55-7.73 (m, 1H), 7.57-7.53 (m, 1H), 7.20-7.10 (m, 3H), 6.96-6.89 (m, 1H), 6.42 (s, 1H), 5.53 (s, 2H), 3.47-3.45 (m, 3H), 2.27 (s, 3H), 1.31 (s, 2H), 1.12-1.11 (m, 2H), 0.86 (t, J=7.1 Hz, 2H), −0.06 (d, J=10.9 Hz, 9H).

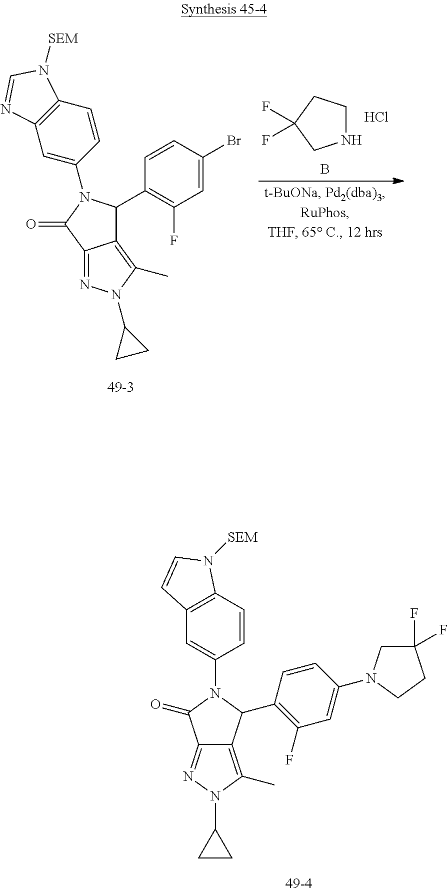

A mixture of compound 49-3 (0.300 g, 502 μmol, 1.00 eq), compound B (144 mg, 1.01 mmol, 2.00 eq, HCl), t-BuONa (193 mg, 2.01 mmol, 4.00 eq), Pd₂(dba)₃ (46.0 mg, 50.2 μmol, 0.100 eq) and RuPhos (46.9 mg, 100 μmol, 0.200 eq) in THF (10 mL) was degassed and purged with N₂ for 3 times, and then the mixture was stirred at 65° C. for 12 hrs under N₂ atmosphere. The reaction mixture was filtered and concentrated under reduced pressure to give a residue, which was purified by reversed-phase HPLC (0.1% NH₃.H₂O in acetonitrile) to give 49-4 (200 mg, 321 μmol, 63.8% yield) as a light yellow solid.

¹H NMR 400 MHz CDCl₃ δ 7.92-7.87 (m, 1H), 7.69-7.67 (m, 2H), 7.60-7.45 (m, 1H), 6.89-6.84 (m, 1H), 6.37-6.32 (m, 1H), 6.15-6.10 (m, 2H), 5.52-5.47 (m, 2H), 3.59-3.42 (m, 7H), 2.47-2.40 (m, 2H), 2.26 (s, 3H), 1.59-1.33 (m, 2H), 1.12-1.09 (m, 2H), 0.91-0.87 (m, 2H), 0.01-−0.06 (m, 9H).

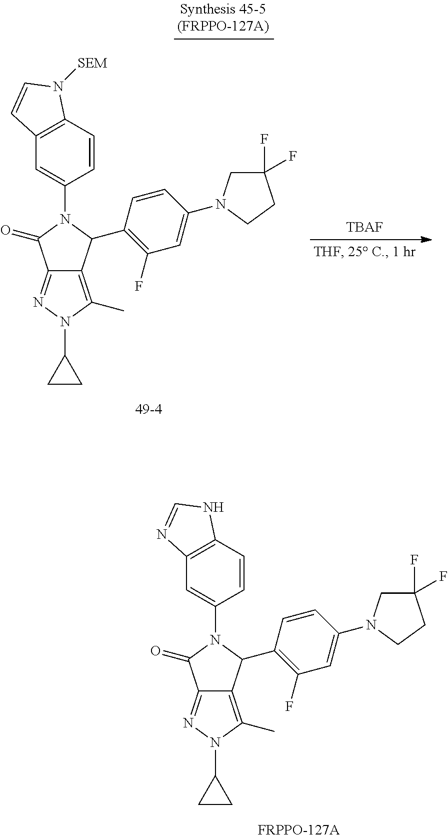 [SMILES: Cc1c2c(nn1C1CC1)C(=O)N(c1ccc3c(ccn3COCC[Si](C)(C)C)c1)C2c1ccc(N2CCC(F)(F)C2)cc1F ; "TBAF" ; Cc1c2c(nn1C1CC1)C(=O)N(c1ccc3[nH]cnc3c1)C2c1ccc(N2CCC(F)(F)C2)cc1F]

To a solution of compound 49-4 (150 mg, 240 μmol, 1.00 eq) in THF (10 mL) was added TBAF (1 M, 481 μL, 2.00 eq). The mixture was stirred at 25° C. for 1 hr. The reaction mixture was concentrated under reduced pressure to give a residue. The crude product was purified by reversed-phase HPLC (0.1% NH₃.H₂O in acetonitrile) to give FRPPO-127A (50.0 mg, 101 μmol, 42.1% yield) as a white solid.

¹H NMR: 400 MHz CDCl₃ δ 7.88 (s, 1H), 7.72-7.27 (m, 2H), 7.18-7.11 (m, 1H), 6.86-6.82 (m, 1H), 6.30 (s, 1H), 6.14-6.08 (m, 2H), 3.58-3.39 (m, 5H), 2.46-2.39 (m, 2H), 2.27 (s, 3H), 1.35-1.31 (m, 2H), 1.14-1.11 (m, 2H).

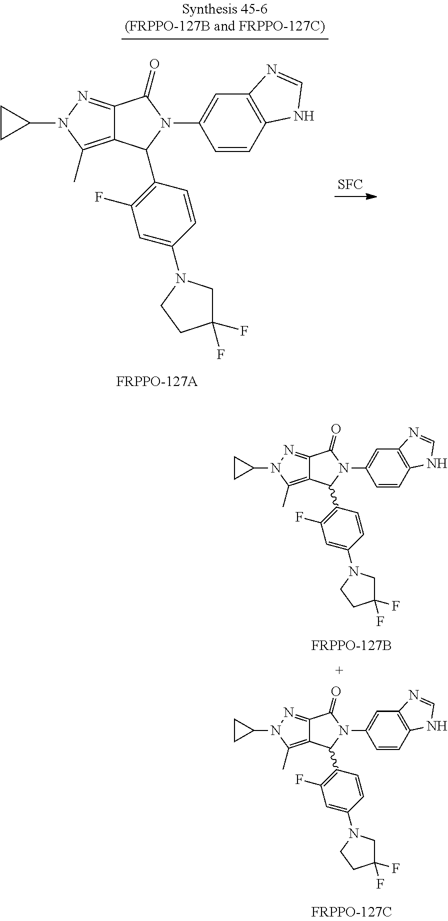 [SMILES: Cc1c2c(nn1C1CC1)C(=O)N(c1ccc3[nH]cnc3c1)C2c1ccc(N2CCC(F)(F)C2)cc1F ; "SFC" ; Cc1c2c(nn1C1CC1)C(=O)N(c1ccc3[nH]cnc3c1)C2c1ccc(N2CCC(F)(F)C2)cc1F ; Cc1c2c(nn1C1CC1)C(=O)N(c1ccc3[nH]cnc3c1)C2c1ccc(N2CCC(F)(F)C2)cc1F]

FRPPO-127A (50.0 mg, 101 μmol, 1.00 eq) was separated by chiral SFC chromatography. Column: Chiralpak AS-3 250×30 mm ID, 10 micron particle size. Mobile phase: 40% A—C02; 60% B—MeOH (0.1% NH₃.H₂O) at 70 g/min, 35° C., to give: FRPPO-127B (8.26 mg, 15.6 μmol, 30.8% yield, 93.3% purity) as a yellow solid.

LCMS: RT=0.993 min, m/z=411.3 (M+1)⁺.

HPLC: RT=2.783 min.

SFC: RT=1.59 min, m/z=411.3 (M+1)⁺.

¹H NMR: 400 MHz MeOD δ 8.15 (s, 1H), 7.61 (s, 1H), 7.54-7.52 (m, 1H), 7.30-7.27 (m, 1H), 6.93 (m, 1H), 6.38 (s, 1H), 6.28-6.19 (m, 2H), 3.63-3.62 (m, 1H), 3.61-3.55 (m, 2H), 3.42-3.31 (m, 2H), 2.45-2.41 (m, 2H), 2.27 (s, 3H), 1.29-1.14 (m, 4H).

FRPPO-127C (10.7 mg, 21.6 μmol, 42.7% yield, 99.8% purity) as a white solid.

LCMS: RT=0.993 min, m/z=411.3 (M+1)⁺.

HPLC: RT=2.905 min.

SFC: RT=2.420 min, m/z=411.3 (M+1)⁺.

¹H NMR: 400 MHz MeOD δ 8.15 (s, 1H), 7.61 (s, 1H), 7.54-7.52 (m, 1H), 7.30-7.27 (m, 1H), 6.93 (m, 1H), 6.38 (s, 1H), 6.28-6.20 (m, 2H), 3.63-3.61 (m, 1H), 3.60-3.55 (m, 2H), 3.40-3.31 (m, 2H), 2.46-2.38 (m, 2H), 2.27 (s, 3H), 1.23-1.14 (m, 4H).

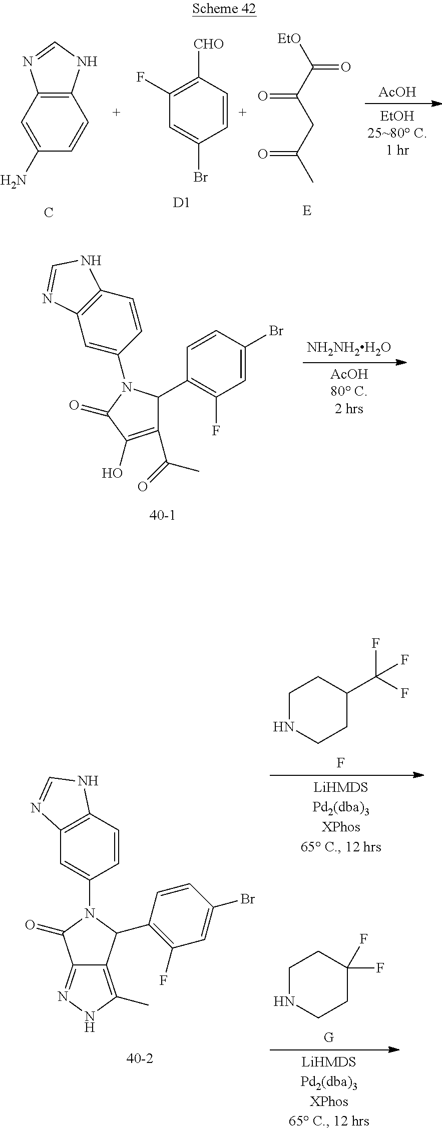

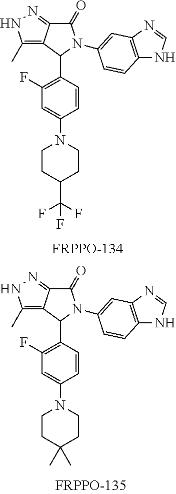 [SMILES: Cc1[nH]nc2c1C(c1ccc(N3CCC(C(F)(F)F)CC3)cc1F)N(c1ccc3[nH]cnc3c1)C2=O ; Cc1[nH]nc2c1C(c1ccc(N3CCC(C)(C)CC3)cc1F)N(c1ccc3[nH]cnc3c1)C2=O]

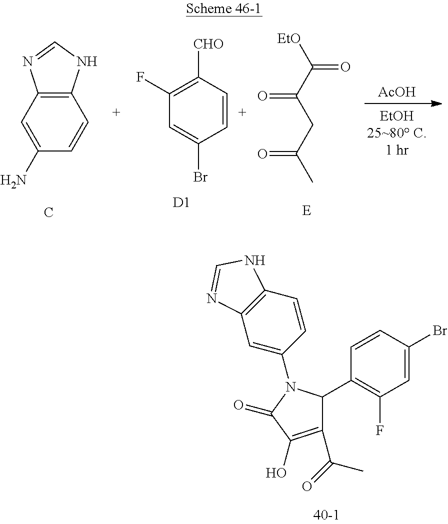

To a solution of compound D1 (15.2 g, 75.1 mmol, 1.00 eq) and compound C (10.0 g, 75.1 mmol, 1.00 eq) in EtOH (100 mL) was added AcOH (4.74 g, 78.8 mmol, 4.51 mL, 1.05 eq). The mixture was stirred at 25° C. for 0.5 hr. And then ethyl compound E (11.8 g, 75.1 mmol, 10.6 mL, 1.00 eq) was added, the mixture was stirred at 80° C. for 0.5 hr. The reaction mixture was cooled to 25° C., and filtered and concentrated under reduced pressure to give compound 40-1 (25.8 g, 59.9 mmol, 79.8% yield) as a yellow solid.

¹H NMR: 400 MHz DMSO-d6 δ 8.25 (s, 1H), 7.71 (d, J=1.5 Hz, 1H), 7.52 (d, J=8.6 Hz, 1H), 7.37-7.29 (m, 2H), 7.23 (s, 2H), 6.23 (s, 1H), 2.36-2.34 (m, 3H).

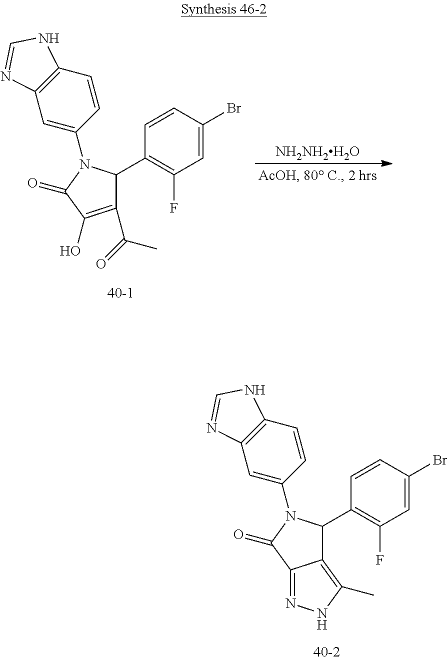 [SMILES: CC(=O)C1=C(O)C(=O)N(c2ccc3[nH]cnc3c2)C1c1ccc(Br)cc1F ; "NH2NH2·H2O" ; Cc1[nH]nc2c1C(c1ccc(Br)cc1F)N(c1ccc3[nH]cnc3c1)C2=O]

To a solution of 40-1 (5.00 g, 11.6 mmol, 1.00 eq) in AcOH (10 mL) was added N₂.H₂H₂O (1.96 g, 38.3 mmol, 1.90 mL, 3.30 eq). The mixture was stirred at 80° C. for 2 hrs. The reaction mixture was quenched by addition water 10 mL at 25° C. and then concentrated under reduced pressure to remove AcOH. And poured into water 100 mL and filtered and concentrated under reduced pressure to give compound 40-2 (5.50 g, crude) as a yellow solid.

¹H NMR: 400 MHz DMSO-d6 δ 8.17 (s, 1H), 7.70 (d, J=1.8 Hz, 1H), 7.52-7.49 (m, 2H), 7.31-7.21 (m, 3H), 6.61 (s, 1H), 2.10 (s, 3H).

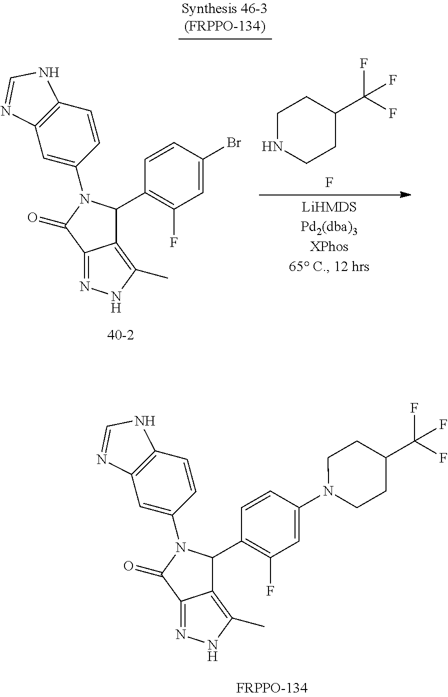

To a solution of 40-2 (200 mg, 469 μmol, 1.00 eq) and compound F (107 mg, 703 μmol, 1.50 eq) in THF (10 mL) was added Pd₂(dba)₃ (21.4 mg, 23.4 μmol, 0.0500 eq) XPhos (22.3 mg, 46.9 μmol, 0.100 eq) and LiHMDS (1 M, 2.35 mL, 5.00 eq). The mixture was stirred at 65° C. for 12 hrs. The reaction mixture was concentrated under reduced pressure to give a residue. The crude product was purified by reversed-phase HPLC (0.1% NH₃.H₂O in acetonitrile) to give FRPPO-134 (49.3 mg, 97.1 μmol, 20.6% yield, 98.0% purity) as a brown solid.

LCMS: RT=0.916 min, m/z=499.4 (M+1)⁺.

HPLC: RT=2.842 min.

¹H NMR: 400 MHz MeOD δ 8.15 (s, 1H), 7.64 (d, J=1.7 Hz, 1H), 7.59-7.45 (m, 1H), 7.33-7.25 (m, 1H), 6.94 (t, J=8.7 Hz, 1H), 6.63-6.55 (m, 2H), 6.41 (s, 1H), 3.73 (d, J=12.2 Hz, 2H), 2.65 (s, 2H), 2.37-2.23 (m, 1H), 2.18 (s, 3H), 1.88 (d, J=12.8 Hz, 2H), 1.58 (s, 2H).

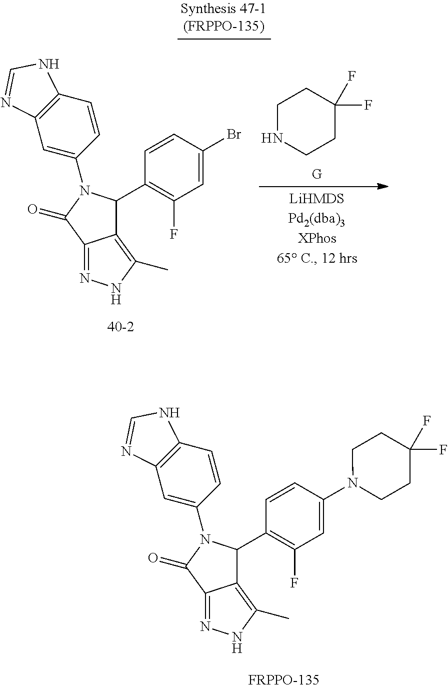

To a solution of compound 40-2 (200 mg, 469 μmol, 1.00 eq) and compound G (110 mg, 703 μmol, 1.50 eq, HCl) in THF (10 mL) was added Pd₂(dba)₃ (21.4 mg, 23.4 μmol, 0.0500 eq) XPhos (22.3 mg, 46.9 μmol, 0.100 eq) and LiHMDS (1 M, 2.35 mL, 5.00 eq). The mixture was stirred at 65° C. for 1 hr. The reaction mixture was concentrated under reduced pressure to give a residue. The crude product was purified by reversed-phase HPLC (0.1% NH₃.H₂O in acetonitrile) to give FRPPO-135 (39.4 mg, 81.0 μmol, 17.2% yield, 96.0% purity) as a yellow solid.

LCMS: RT=0.878 min, m/z=467.4 (M+1)⁺.

HPLC: RT=2.553 min.

¹H NMR: 400 MHz MeOD δ 8.15 (s, 1H), 7.64 (d, J=1.8 Hz, 1H), 7.55 (d, J=8.7 Hz, 1H), 7.31 (dd, J=1.9, 8.6 Hz, 1H), 6.96 (t, J=8.7 Hz, 1H), 6.71-6.59 (m, 2H), 6.42 (s, 1H), 3.31 (s, 4H), 2.18 (s, 3H), 1.98-1.94 (m, 4H).

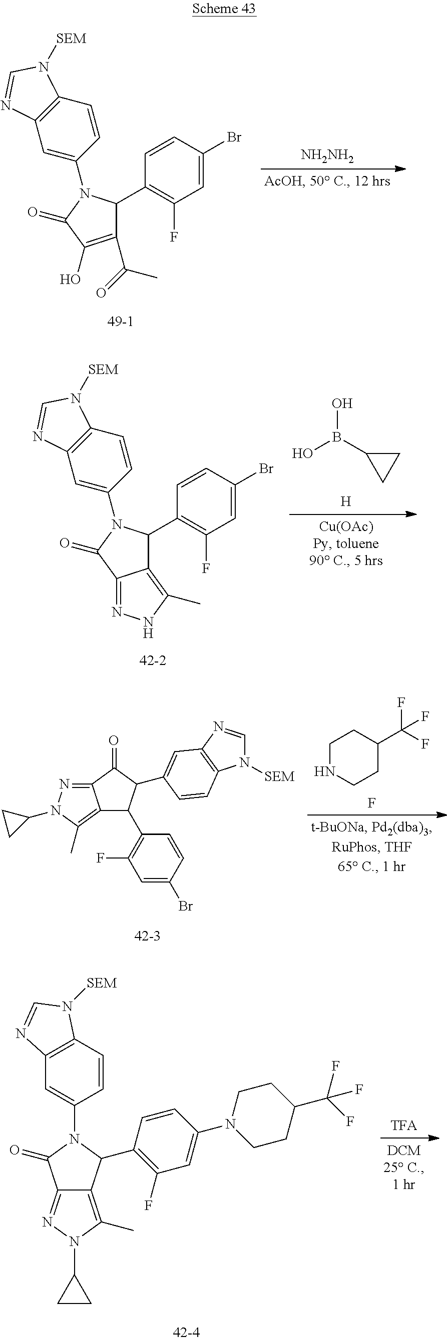

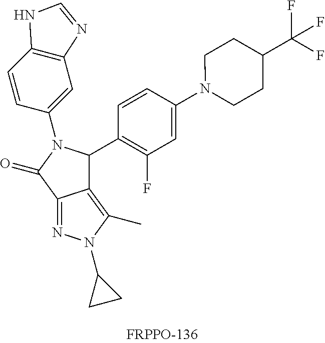 [SMILES: Cc1c2c(nn1C1CC1)C(=O)N(c1ccc3[nH]cnc3c1)C2c1ccc(N2CCC(C(F)(F)F)CC2)cc1F]

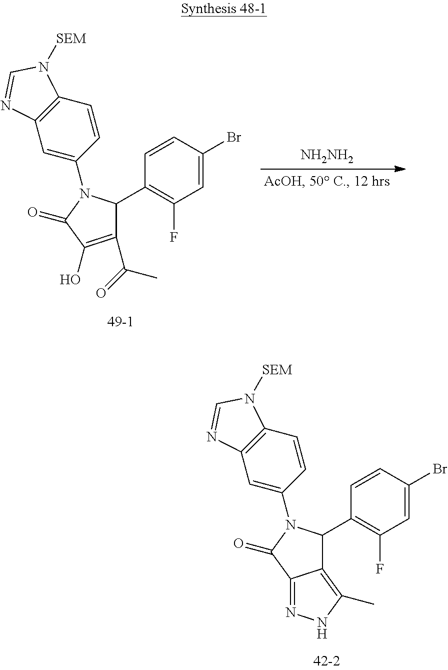 [SMILES: CC(=O)C1=C(O)C(=O)N(c2ccc3c(c2)ncn3COCC[Si](C)(C)C)C1c1ccc(Br)cc1F ; "NH2NH2" ; Cc1[nH]nc2c1C(c1ccc(Br)cc1F)N(c1ccc3c(c1)ncn3COCC[Si](C)(C)C)C2=O]

To a solution of compound 49-1 (Scheme 41 and Synthesis 45-1) (8.00 g, 14.2 mmol, 1.00 eq) in AcOH (30 mL) was added hydrazine (1.44 g, 44.9 mmol, 1.63 mL, 3.15 eq). The mixture was stirred at 50° C. for 12 hrs. The reaction mixture was diluted with water 30 mL and extracted with EtOAc 150 mL (50 mL*3). The combined organic layers were washed with water 100 mL (50 mL*2) and saturated NaHCO₃ 100 mL (50 mL*2) concentrated under reduced pressure to give compound 42-2 (9.00 g, crude) w as a red solid.

¹H NMR: 400 MHz CDCl₃ δ 7.96 (d, J=1.6 Hz, 1H), 7.75 (d, J=1.7 Hz, 1H), 7.54-7.51 (m, 1H), 7.31-7.27 (m, 1H), 7.21-7.19 (m, 2H), 7.18-6.93 (m, 1H), 6.45-6.42 (m, 1H), 5.56-5.45 (m, 2H), 3.51-3.47 (m, 2H), 2.28 (s, 3H), 0.91-0.87 (m, 2H), 0.03-−0.07 (m, 8H).

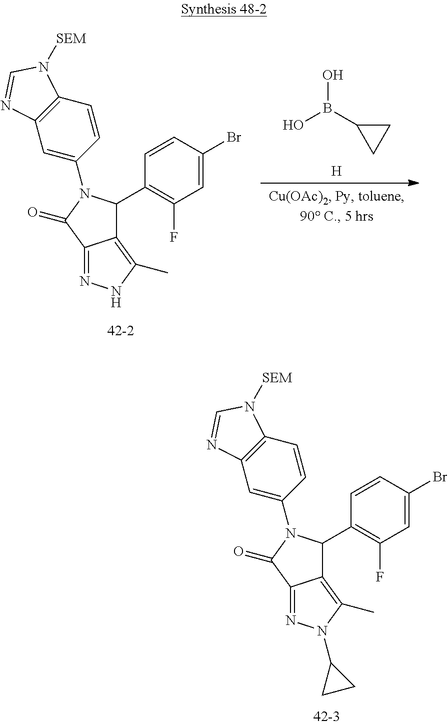

To a solution of compound 42-2 (0.550 g, 988 μmol, 1.00 eq) and compound 42-3 (110 mg, 1.28 mmol, 1.30 eq) in toluene (5 mL) was added Cu(OAc)₂ (269 mg, 1.48 mmol, 1.50 eq) and Py (234 mg, 2.96 mmol, 239 μL, 3.00 eq). The mixture was degassed and purged with O₂ for 3 times, and then the mixture was stirred at 90° C. for 5 hrs under O₂ atmosphere (15 Psi). LCMS showed desired mass was detected. The reaction mixture was filtered and concentrated under reduced pressure to give a residue. The crude product was purified by reversed-phase HPLC (0.1% NH₃.H₂O) to give compound 42-3 (0.06 g, 100 μmol, 10.1% yield) as a yellow oil.

¹H NMR: 400 MHz CDCl₃ δ 8.55-7.73 (m, 1H), 7.57-7.53 (m, 1H), 7.20-7.10 (m, 3H), 6.96-6.89 (m, 1H), 6.42 (s, 1H), 5.53 (s, 2H), 3.47-3.45 (m, 3H), 2.27 (s, 3H), 1.31 (s, 2H), 1.12-1.11 (m, 2H), 0.86 (t, J=7.1 Hz, 2H), −0.06 (d, J=10.9 Hz, 9H).

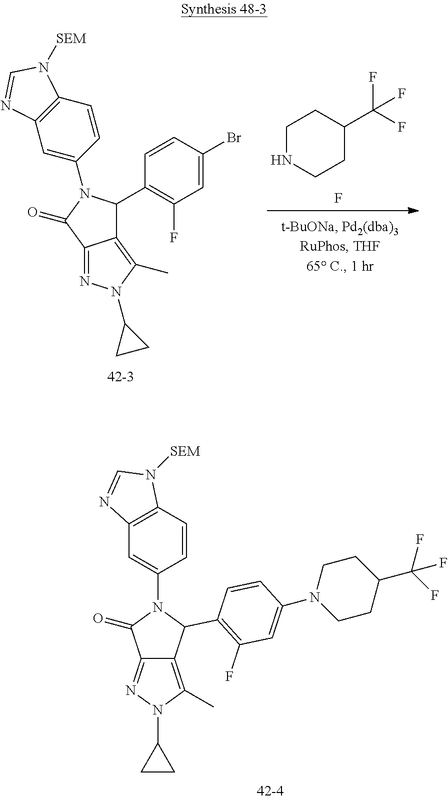

To a solution of compound 42-3 (100 mg, 167 μmol, 1.00 eq) and compound F (51.3 mg, 335 μmol, 2.00 eq) in THF (5 mL) was added Pd₂(dba)₃ (15.3 mg, 16.7 μmol, 0.100 eq), RuPhos (15.6 mg, 33.5 μmol, 0.200 eq) and t-BuONa (64.4 mg, 670 μmol, 4.00 eq). The mixture was stirred at 65° C. for 1 hr. The reaction mixture was filtered and concentrated under reduced pressure to give a residue. The crude product was purified by reversed-phase HPLC (0.1% NH₃.H₂O in acetonitrile) to give compound 42-4 (50.0 mg, 74.7 μmol, 44.6% yield) as a yellow oil.

LCMS: RT=1.203 min, m/z=669.3 (M+1)⁺.

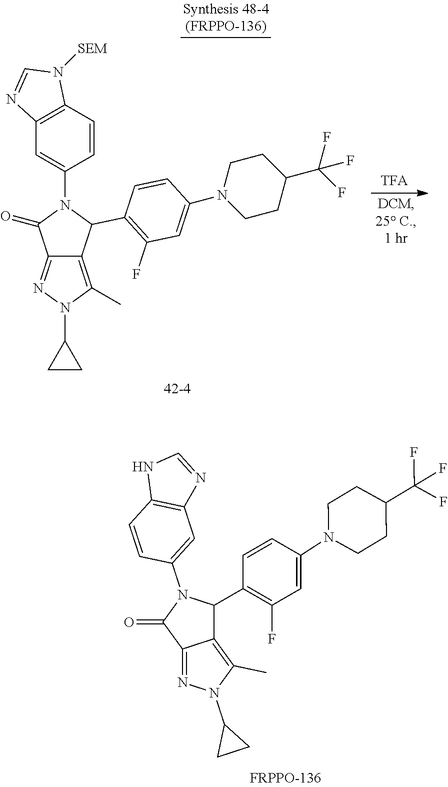

To a solution of compound 42-4 (50.0 mg, 74.7 μmol, 1.00 eq) in DCM (5 mL) was added TFA (1.54 g, 13.5 mmol, 1 mL, 180 eq). The mixture was stirred at 25° C. for 1 hr. The reaction mixture was adjust pH to 7.00 with NH₃.H₂O, and filtered and concentrated under reduced pressure to give a residue. The residue was purified by prep-HPLC (basic condition, column: Xtimate C18 150*40 mm*10 μm; mobile phase: 0.05% ammonia hydroxide, A—water, B—ACN; gradient: 37%-67% B, 10 min) to give FRPPO-136 (6.44 mg, 11.9 μmol, 16.0% yield, 100% purity) as an off-white solid.

LCMS: RT=2.455 min, m/z=539.3 (M+1)⁺

¹H NMR: 400 MHz CDCl₃ δ 7.93 (s, 1H), 7.80 (s, 1H), 7.53 (s, 1H), 7.25-7.21 (m, 1H), 6.85 (t, J=8.7 Hz, 1H), 6.51-6.46 (m, 2H), 6.33 (s, 1H), 3.69-3.66 (m, 2H), 3.48-3.46 (m, 1H), 2.69-2.62 (m, 2H), 2.27 (s, 3H), 1.94-1.90 (m, 1H), 1.65-1.58 (m, 2H), 1.35-1.33 (s, 2H), 1.33-1.32 (m, 2H), 1.13-1.11 (m, 2H).

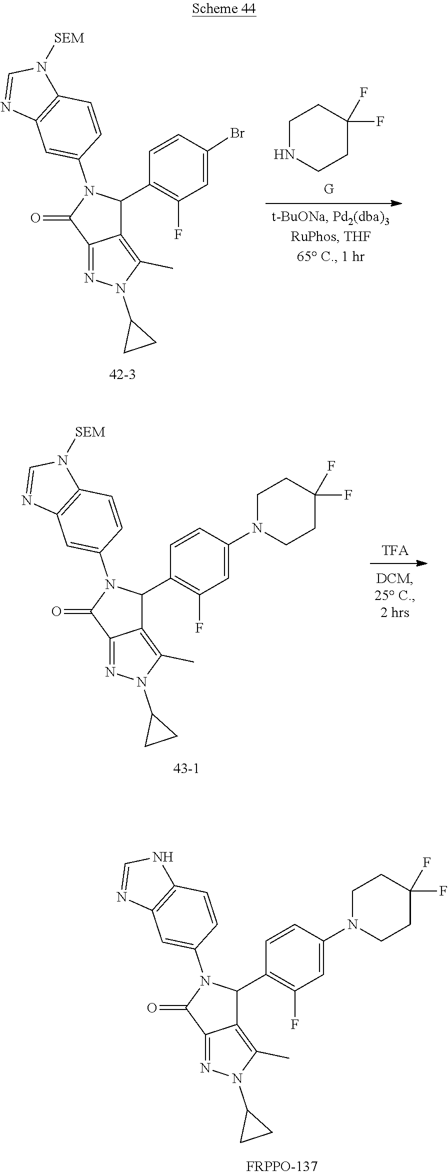 [SMILES: Cc1c2c(nn1C1CC1)C(=O)N(c1ccc3c(c1)ncn3COCC[Si](C)(C)C)C2c1ccc(Br)cc1F ; FC1(F)CCNCC1 ; Cc1c2c(nn1C1CC1)C(=O)N(c1ccc3c(c1)ncn3COCC[Si](C)(C)C)C2c1ccc(N2CCC(F)(F)CC2)cc1F ; Cc1c2c(nn1C1CC1)C(=O)N(c1ccc3[nH]cnc3c1)C2c1ccc(N2CCC(F)(F)CC2)cc1F]

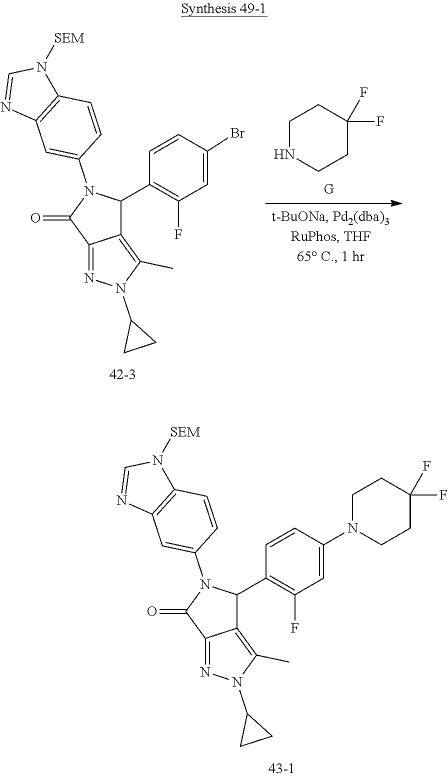 [SMILES: Cc1c2c(nn1C1CC1)C(=O)N(c1ccc3c(c1)ncn3COCC[Si](C)(C)C)C2c1ccc(Br)cc1F ; FC1(F)CCNCC1 ; Cc1c2c(nn1C1CC1)C(=O)N(c1ccc3c(c1)ncn3COCC[Si](C)(C)C)C2c1ccc(N2CCC(F)(F)CC2)cc1F]

To a solution of compound 42-3 (Scheme 43 and Synthesis 48-2) (0.0600 g, 100 μmol, 1.00 eq) and compound G (24.3 mg, 154 μmol, 1.54 eq, HCl) in THF (5 mL) was added t-BuONa (38.6 mg, 402 μmol, 4.00 eq), RuPhos (9.39 mg, 20.1 μmol, 0.200 eq) and Pd₂(dba)₃ (9.21 mg, 10.0 μmol, 0.100 eq). The mixture was stirred at 65° C. for 1 hr. The reaction mixture was filtered and concentrated under reduced pressure to give a residue. The crude product was purified by reversed-phase HPLC (0.1% NH₃.H₂O in acetonitrile) to give compound 43-1 (40.0 mg, 62.82 μmol, 62.46% yield) as a yellow solid.

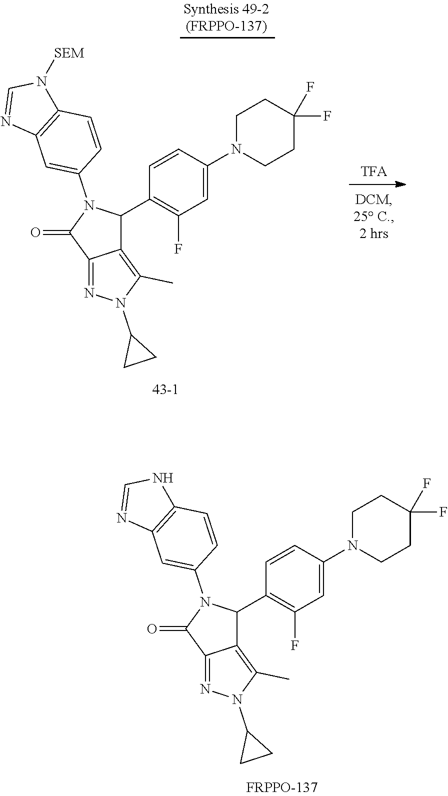

To a solution of compound 43-1 (40.0 mg, 62.8 μmol, 1.00 eq) in DCM (4 mL) was added TFA (1.54 g, 13.5 mmol, 1 mL, 215 eq). The mixture was stirred at 25° C. for 2 hr. LCMS (EW19087-34-P1B) showed desired mass was detected. The reaction mixture was filtered and concentrated under reduced pressure to give a residue. The residue was purified by prep-HPLC (basic condition, column: Xtimate C18 150*40 mm*10 μm; mobile phase: 0.05% ammonia hydroxide, A—water, B—ACN; gradient: 35%-55% B, 10 min) to give FRPPO-137 (4.73 mg, 9.33 μmol, 14.8% yield, 99.9% purity) as a white solid.

LCMS: RT=2.312 min, m/z=507.3 (M+1)⁺

HPLC: RT=2.983 min

¹H NMR: 400 MHz CDCl₃ δ 7.93 (s, 1H), 7.88-7.87 (m, 1H), 7.67-7.66 (m, 1H), 7.13-7.10 (m, 1H), 6.86-6.84 (m, 1H), 6.51-6.47 (m, 2H), 6.34 (s, 1H), 3.47 (qd, J=3.7, 7.3 Hz, 1H), 3.30-3.28 (m, 4H), 2.28 (s, 3H), 2.06-1.96 (m, 4H), 1.35-1.32 (m, 2H), 1.12 (d, J=7.1 Hz, 2H).

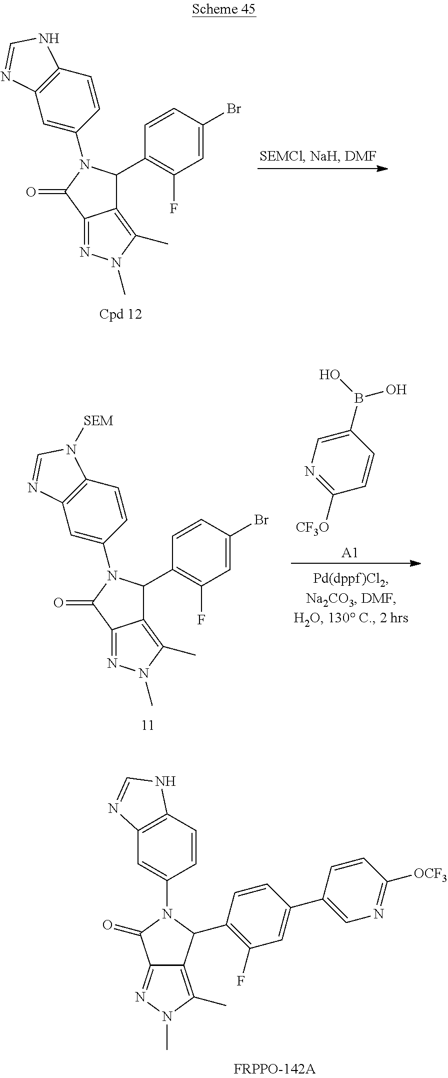

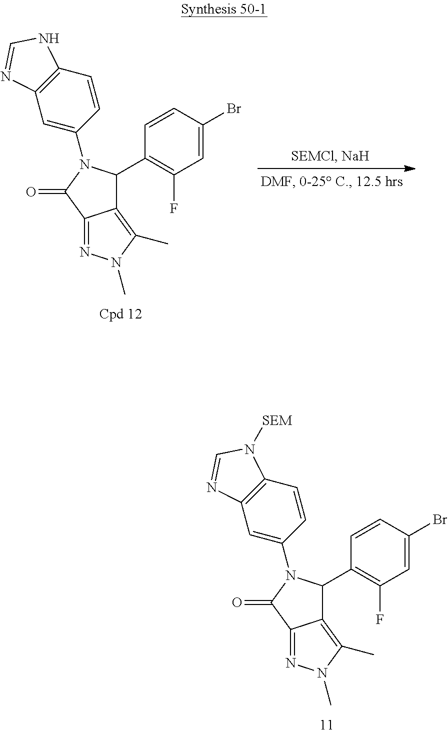 [SMILES: Cc1c2c(nn1C)C(=O)N(c1ccc3[nH]cnc3c1)C2c1ccc(Br)cc1F ; Cc1c2c(nn1C)C(=O)N(c1ccc3c(c1)ncn3COCC[Si](C)(C)C)C2c1ccc(Br)cc1F]

To a solution of compound Cpd 12 (Scheme 4 and Synthesis 5-2) (2.00 g, 4.54 mmol, 1.00 eq) in DMF (10 mL) was added NaH (250 mg, 6.25 mmol, 60% purity, 1.38 eq). The mixture was stirred at 0° C. for 0.5 hr, then SEM-Cl (942 mg, 5.65 mmol, 1 mL, 1.24 eq) was added, the mixture was stirred at 25° C. for 12 hrs. LCMS (EW20960-22-P1A) showed compound 5 was consumed, desired m/z=572.4 (RT=1.086 min) was detected. The reaction mixture was quenched by addition water 10 mL at 25° C., and then diluted with water 100 mL and extracted with Ethyl acetate 300 mL (100 mL*3). The combined organic layers were washed with water 200 mL (100 mL*2) and brine 150 mL, dried over Na₂SO₄, filtered and concentrated under reduced pressure to give a residue. The crude product was purified by reversed-phase HPLC (0.1% NH₃.H₂O) to give compound 11 (1.60 g, 2.80 mmol, 61.7% yield) as a yellow solid.

LCMS: RT=1.086 min, m/z=572.4 (M+1)⁺.

¹H NMR: 400 MHz CDCl₃ δ 7.93-7.86 (m, 1H), 7.71-7.69 (m, 2H), 7.49-7.47 (m, 1H), 7.29-7.21 (m, 1H), 7.12-7.10 (m, 1H), 6.95-6.92 (m, 1H), 6.43-6.41 (m, 1H), 5.56-5.44 (m, 2H), 3.92 (s, 3H), 3.53-3.47 (m, 2H), 2.18 (s, 3H), 0.91-0.87 (m, 2H), 0.02-0.01 (m, 9H).

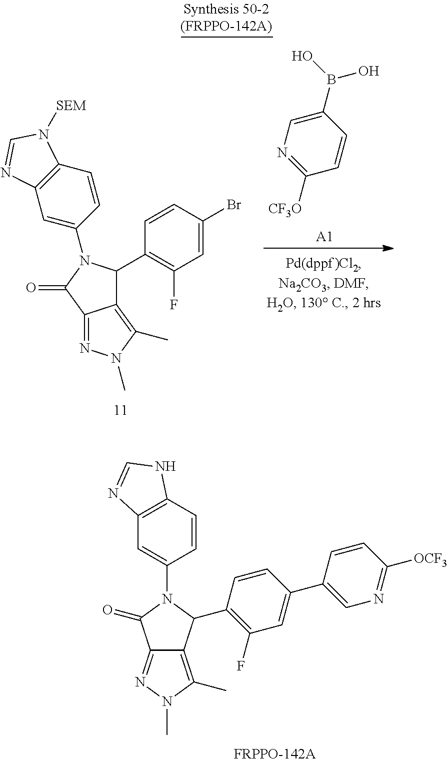

To a solution of compound 11 (50 mg, 113 μmol, 1.00 eq) and compound A1 (50 mg, 241 μmol, 2.13 eq) in H₂O (0.5 mL) and DMF (5 mL) was added Pd(dppf)Cl₂ (10 mg, 13.6 μmol, 0.12 eq) and Na₂CO₃ (40 mg, 377 μmol, 3.32 eq). The mixture was stirred at 130° C. for 2 hrs under microwave. The reaction mixture was partitioned between water 100 mL and Ethyl acetate 150 mL. The organic phase was separated, washed with water 100 mL (50 mL*2) and brine 100 mL, dried over Na₂SO₄, filtered and concentrated under reduced pressure to give a residue. The residue was purified by prep-HPLC prep-HPLC (basic condition, column: Waters Xbridge 150*25 mm*5 μm; mobile phase: 0.05% ammonia hydroxide, A—water, B—ACN; gradient: 34%-54% B, 10 min) to give FRPPO-142A (11.94 mg, 22.8 μmol, 20.1% yield, 100% purity) as a off-white solid.

LCMS: RT=0.965 min, m/z=523.4 (M+1)⁺.

HPLC: RT=2.229 min

¹H NMR: 400 MHz DMSO-d6 δ 8.65-8.64 (m, 2H), 8.29-8.26 (m, 1H), 7.91 (s, 1H), 7.64-7.61 (m, 2H), 7.54-7.53 (m, 2H), 7.48-7.47 (m, 1H), 7.35-7.33 (m, 1H), 6.77 (s, 1H), 3.89 (s, 3H), 2.17 (s, 3H).

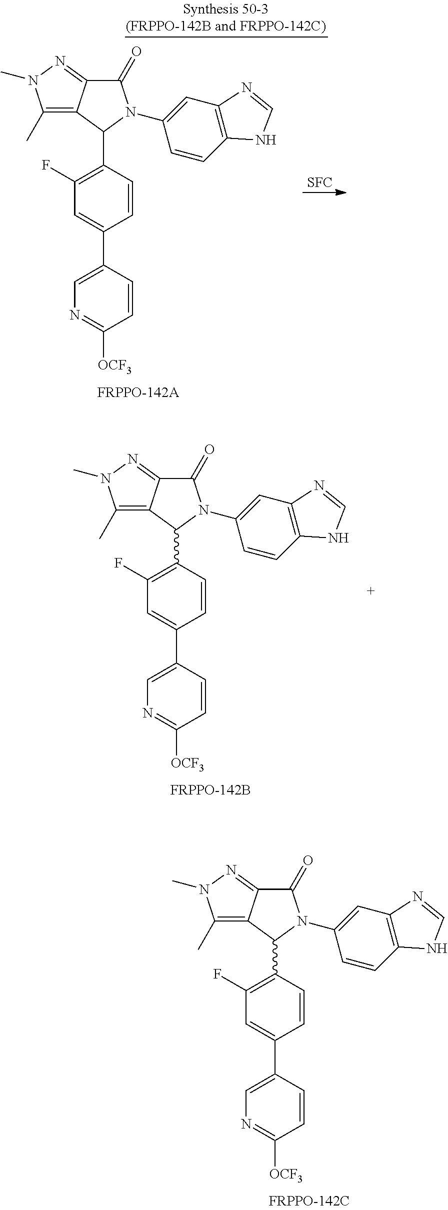

FRPPO-142A (11.94 mg, 22.8 μmol, 1.00 eq) was separated by SFC (basic condition column: DAICEL CHIRALPAK AD (250 mm*30 mm, 10 μm); mobile phase: [0.1% NH₃.H₂O ETOH]; B %: 40%-40%, 6.2 min; 70 min) to give:

FRPPO-142B (7.31 mg, 13.8 μmol, 60.6% yield, 99.1% purity) as an off-white solid.

LCMS: RT=0.940 min, m/z=496.3 (M+1)⁺.

HPLC: RT=1.956 min.

SFC: RT=1.333 min.

¹H NMR: 400 MHz DMSO-d6 δ 12.4 (s, 1H), 9.12 (s, 1H), 8.18-8.17 (m, 2H), 7.75-7.71 (m, 2H), 7.65-7.62 (m, 1H), 7.46-7.44 (m, 2H), 7.43-7.31 (m, 1H), 6.68-6.66 (m, 1H), 3.88 (s, 3H), 2.15 (s, 3H).

FRPPO-142C (7.79 mg, 14.51 μmol, 63.48% yield, 97.3% purity) as an off-white solid.

LCMS: RT=0.938 min, m/z=496.3 (M+1)⁺.

HPLC: RT=1.959 min.

SFC: RT=1.670 min.

¹H NMR: 400 MHz DMSO-d6 δ 12.4 (s, 1H), 9.12 (s, 1H), 8.18-8.17 (m, 2H), 7.75-7.71 (m, 2H), 7.65-7.62 (m, 1H), 7.46-7.44 (m, 2H), 7.43-7.31 (m, 1H), 6.68-6.66 (m, 1H), 3.88 (s, 3H), 2.15 (s, 3H).

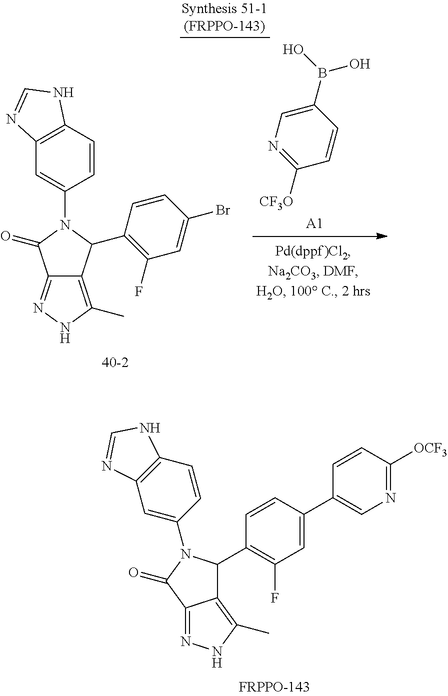

To a solution of 40-2 (Scheme 42 and Synthesis 46-2) (50.0 mg, 117 μmol, 1.00 eq) and compound A1 (50.0 mg, 242 μmol, 2.06 eq) in H₂O (0.5 mL) and DMF (5 mL) was added Pd(dppf)Cl₂ (20.0 mg, 27.3 μmol, 0.2 eq) and Na₂CO₃ (40.0 mg, 377 μmol, 3.22 eq). The mixture was stirred at 100° C. for 2 hrs under microwave. The mixture was filtrated and the filtrate was concentrated to give a residue. The residue was purified by prep-HPLC (basic condition, column: Xtimate C18 150*40 mm*10 μm; mobile phase: 0.05% ammonia hydroxide, A—water, B—ACN; gradient: 28%-58% B, 10 min) to give FRPPO-143 (16.03 mg, 31.5 μmol, 26.8% yield, 99.8% purity) as a white solid.

LCMS: RT=0.922 min, m/z=509.3 (M+1)⁺.

HPLC: RT=2.612 min.

¹H NMR: 400 MHz DMSO-d6 δ 13.38 (s, 1H), 12.46 (s, 1H), 8.65 (s, 1H), 8.29-8.27 (m, 1H), 8.26-8.19 (m, 1H), 7.79-7.78 (m, 1H), 7.64-7.60 (m, 3H), 7.49-7.33 (s, 3H), 6.73-6.71 (m, 1H), 2.14 (s, 3H).

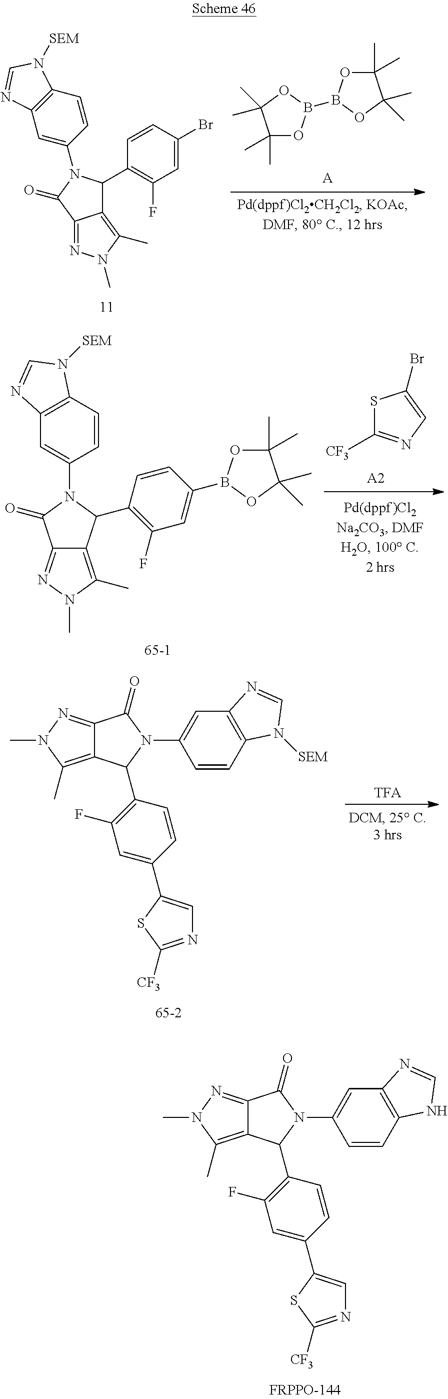

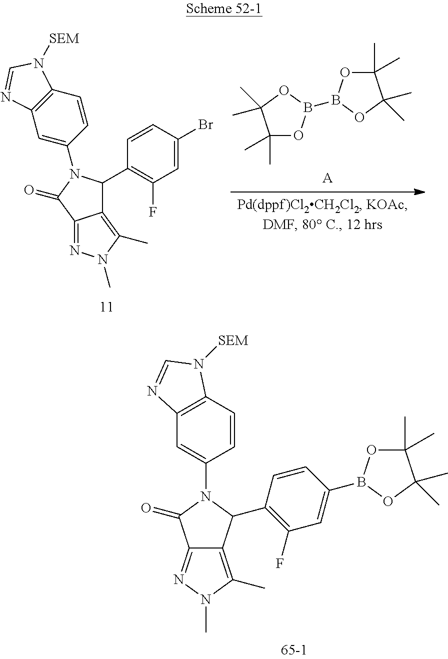

To a solution of compound 11 (Scheme 45 and Synthesis 50-1) (700 mg, 1.23 mmol, 1.00 eq) and compound A (470 mg, 1.85 mmol, 1.51 eq) in DMF (8 mL) was added KOAc (378 mg, 3.85 mmol, 3.14 eq) and Pd(dppf)Cl₂.CH₂Cl₂ (60.0 mg, 73.4 μmol, 0.05 eq). The mixture was stirred at 80° C. for 12 hrs. The reaction mixture was filtered and filtrate was diluted with H₂O 30 mL and EtOAc (30 mL*3). The combined organic layers were washed with brine (20 mL*2), dried over Na₂SO₄, filtered and concentrated under reduced pressure to give a residue. The crude product was purified by reversed-phase HPLC (0.1% FA condition) to get compound 65-1 (1.20 g, crude) as a black brown oil.

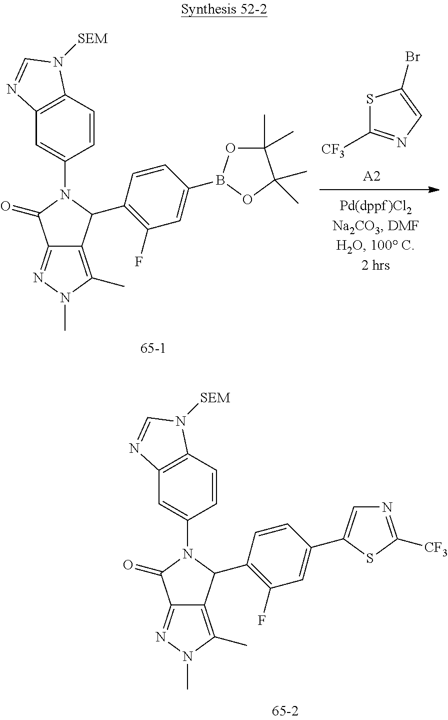

To a solution of compound 65-1 (150 mg, 243 μmol, 1.13 eq) and compound A2 (50.0 mg, 216 μmol, 1.00 eq) in DMF (2 mL) and H₂O (0.2 mL) was added Na₂CO₃ (70.0 mg, 660 μmol, 3.06 eq) and Pd(dppf)Cl₂ (47.3 mg, 64.7 μmol, 0.30 eq). The mixture was stirred at 100° C. for 2 hrs. The mixture was filtrated and the filtrate was concentrated to give a residue. The crude product was purified by reversed-phase HPLC (0.1% FA condition) to give compound 65-2 (30.0 mg, crude) as a brown solid.

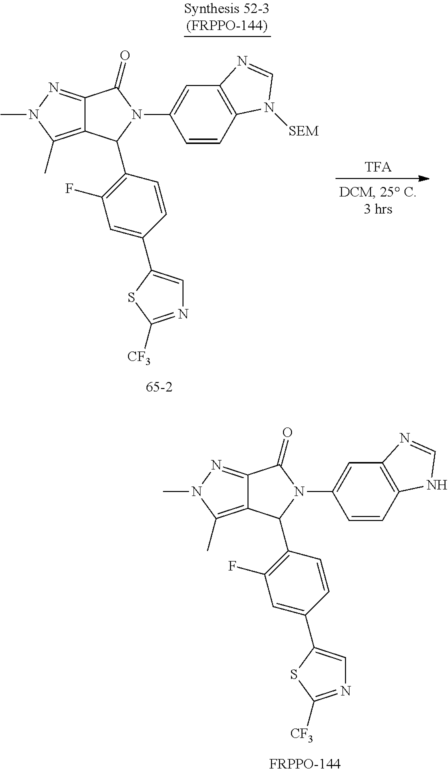

To a solution of 65-2 (30.0 mg, 46.7 μmol, 1.00 eq) in DCM (1 mL) was added TFA (1.54 g, 13.5 mmol, 1 mL, 289 eq). The mixture was stirred at 25° C. for 3 hrs. The mixture was diluted with DCM (30 mL) and adjusted to pH=8 with Na₂CO₃ solution, the organic layer was dried over anhydrous sodium sulfate, filtrated and concentrated to give a residue. The residue was purified by prep-HPLC (basic condition, column: Waters Xbridge 150*25 mm*5 μm; mobile phase: 0.05% ammonia hydroxide, A—water, B—ACN; gradient: 28%-58% B, 10 min) to get FRPPO-144 (7.27 mg, 13.4 μmol, 28.7% yield, 94.5% purity) as an off-white solid.

LCMS: RT=0.934 min, m/z=513.2 (M+1)⁺.

HPLC: RT=2.940 min.

¹H NMR: 400 MHz DMSO-d6 δ 8.51 (s, 1H), 8.18-8.16 (m, 1H), 7.75-7.68 (m, 2H), 7.50-7.36 (m, 4H), 6.70-6.69 (m, 1H), 3.88-0.87 (m, 3H), 2.16-2.15 (m, 3H).

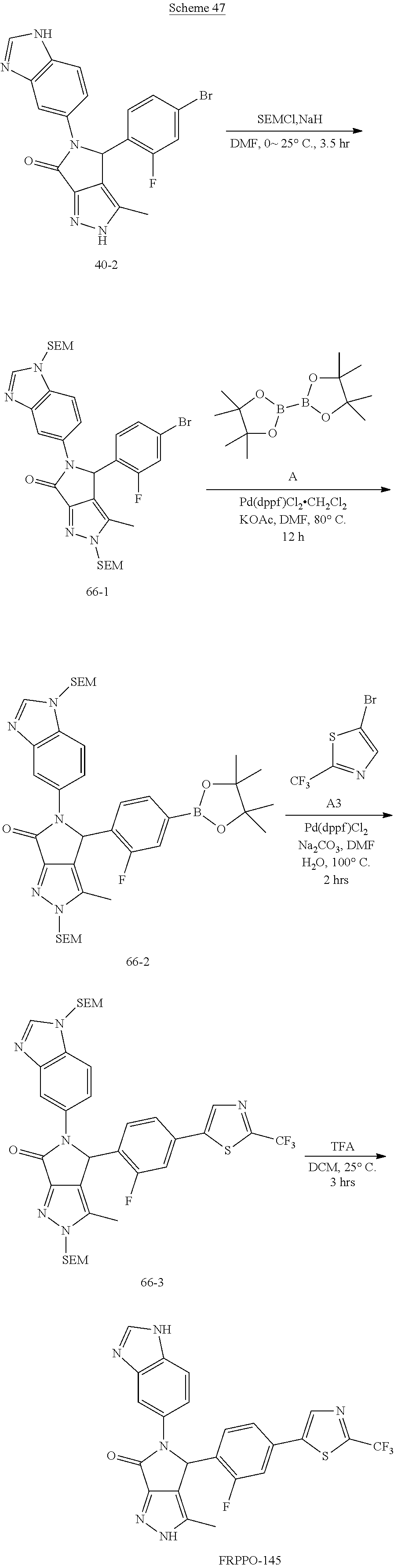

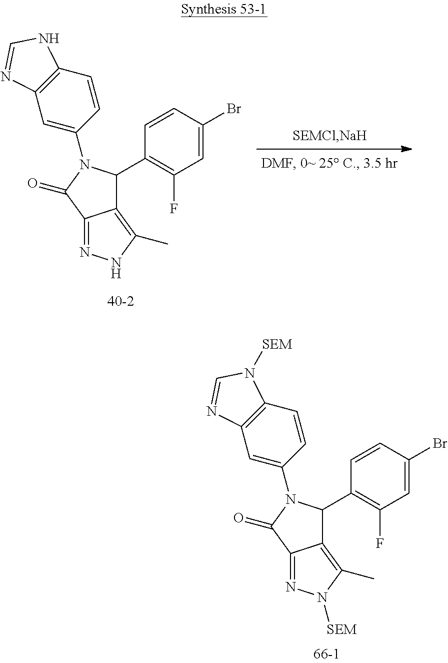 [SMILES: Cc1[nH]nc2c1C(c1ccc(Br)cc1F)N(c1ccc3[nH]cnc3c1)C2=O ; Cc1c2c(nn1COCC[Si](C)(C)C)C(=O)N(c1ccc3c(c1)ncn3COCC[Si](C)(C)C)C2c1ccc(Br)cc1F]

To a solution of compound 40-2 (Scheme 42 and Synthesis 46-2) (900 mg, 2.07 mmol, 1.00 eq) in DMF (10 mL) was added NaH (170 mg, 4.25 mmol, 60% purity, 2.06 eq) at 0° C. The mixture was stirred at 0° C. for 0.5 hr, then SEM-Cl (848 mg, 5.09 mmol, 900 μL, 2.46 eq) was added and the mixture was stirred at 25° C. for 3 hrs. The mixture was poured into water (30 mL) and extracted with EtOAc (30 mL*2). The combined organic layers were dried over anhydrous sodium sulfate, filtrated and concentrated to give a residue. The crude product was purified by reversed-phase HPLC (0.1% FA in acetonitrile) to give compound 66-1 (650 mg, 941 μmol, 45.6% yield, 99.4% purity) as a brown oil.

LCMS: RT=1.077 min, m/z=688.3 (M+1)⁺.

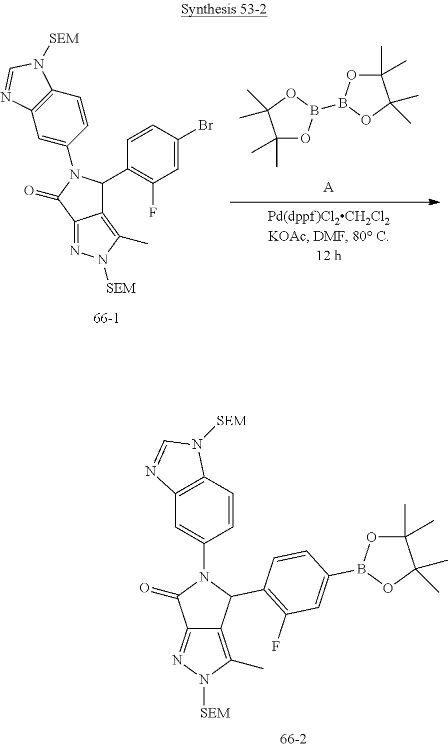

To a solution of 66-1 (850 mg, 1.24 mmol, 1.00 eq) and compound A (943 mg, 3.71 mmol, 3.00 eq) in DMF (10 mL) was added KOAc (370 mg, 3.77 mmol, 3.05 eq) and Pd(dppf)Cl₂.CH₂Cl₂ (50.0 mg, 61.2 μmol, 0.05 eq). The mixture was stirred at 100° C. for 20 hrs. The mixture was filtrated and the filtrate was diluted with EtOAc (30 mL) and washed with brine (50 mL*2). The organic layer was dried over anhydrous sodium sulfate, filtrated and concentrated to give compound 66-2 (1.20 g, crude) as a black oil.

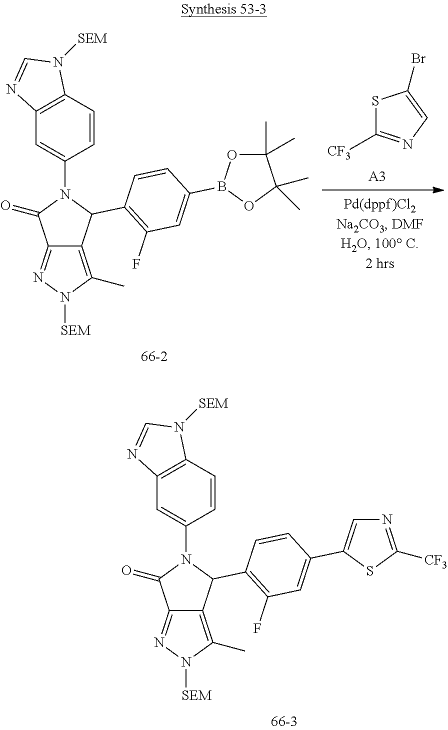

To a solution of compound 66-2 (800 mg, 1.09 mmol, 1.26 eq) and compound A3 (200 mg, 862 μmol, 1.00 eq) in DMF (5 mL) and H₂O (0.5 mL) was added Na₂CO₃ (280 mg, 2.64 mmol, 3.06 eq) and Pd(dppf)Cl₂ (126 mg, 172 μmol, 0.20 eq). The mixture was stirred at 100° C. for 1 hr. The reaction was filtrated and the filtrate was concentrated to give a residue. The crude product was purified by reversed-phase HPLC (0.1% FA in acetonitrile) to give compound 66-3 (100 mg, 63.2 μmol, 7.34% yield, 48% purity) as a yellow solid, which was used in the next step directly.

LCMS: RT=1.130 min, m/z=759.2 (M+1)⁺.

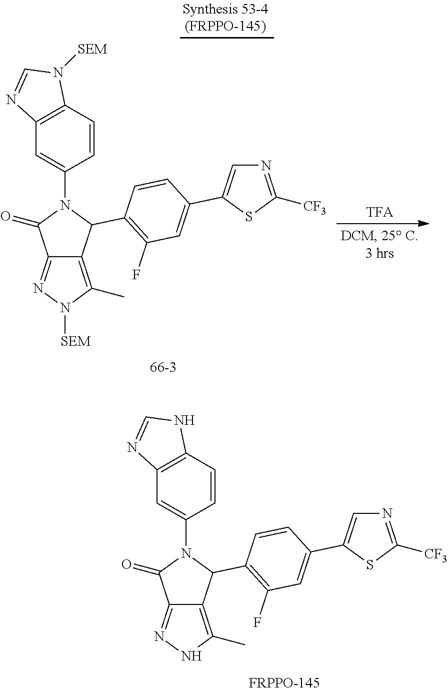

To a solution of compound 66-3 (90.0 mg, 56.9 μmol, 1.00 eq) in DCM (2 mL) was added TFA (3.08 g, 27.0 mmol, 2 mL, 475 eq). The mixture was stirred at 25° C. for 3 hrs. The mixture was diluted with DCM (30 mL) and adjusted to pH˜8 with Na₂CO₃ solution. The mixture was extracted with DCM (15 mL*2). The combined organic layers were dried over anhydrous sodium sulfate, filtrated and concentrated to give a residue. The residue was purified by prep-HPLC (basic condition, column: Waters Xbridge 150*25 mm*5 μm mobile phase: 0.05% ammonia hydroxide, A—water, B—ACN; gradient: 25%-55% B, 10 min) to give FRPPO-145 (4.80 mg, 9.54 μmol, 16.8% yield, 99.1% purity) as a white solid.

LCMS: RT=0.922 min, m/z=499.3 (M+1)⁺.

HPLC: RT=2.511 min.

¹H NMR: 400 MHz DMSO-d6 δ 8.52 (s, 1H), 8.18 (s, 1H), 7.76-7.68 (m, 2H), 7.50-7.40 (m, 2H), 7.38-7.36 (m, 2H), 6.69 (s, 1H), 2.12 (s, 3H).

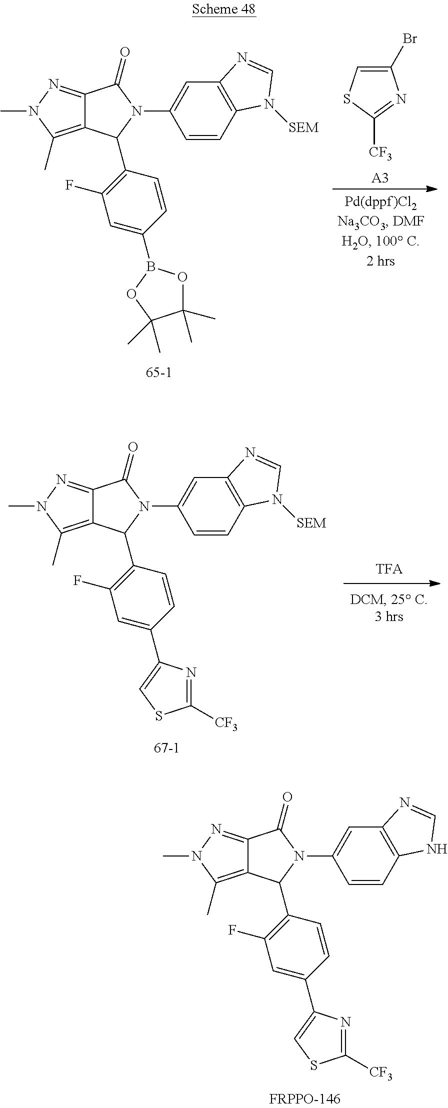

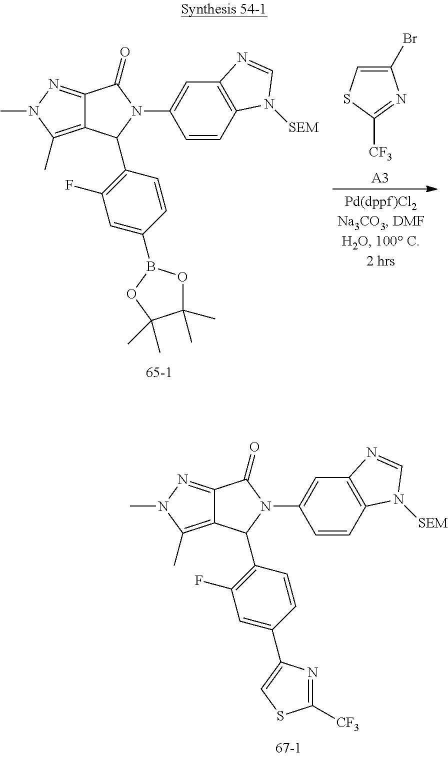

To a solution of compound 65-1 (Scheme 46 and Synthesis 52-1) (200 mg, 323 μmol, 1.00 eq) and A3 (240 mg, 1.03 mmol, 3.19 eq) in DMF (4 mL) and H₂O (1 mL) was added Pd(dppf)Cl₂ (48.0 mg, 65.6 μmol, 0.20 eq) and Na₂CO₃ (104 mg, 981 μmol, 3.03 eq). The mixture was stirred at 100° C. for 2 hrs. The reaction mixture was diluted with water 20 mL and extracted with EtOAc (20 mL*2). The combined organic layers were washed with brine (20 mL*2), dried over Na₂SO₄, filtered and concentrated under reduced pressure to give a residue. The crude product was purified by reversed-phase HPLC (0.1% FA condition) to give compound 67-1 (60 mg, 93.4 μmol, 28.8% yield) as black brown solid.

LCMS: RT=0.887 min, m/z=643.1 (M+1)⁺.

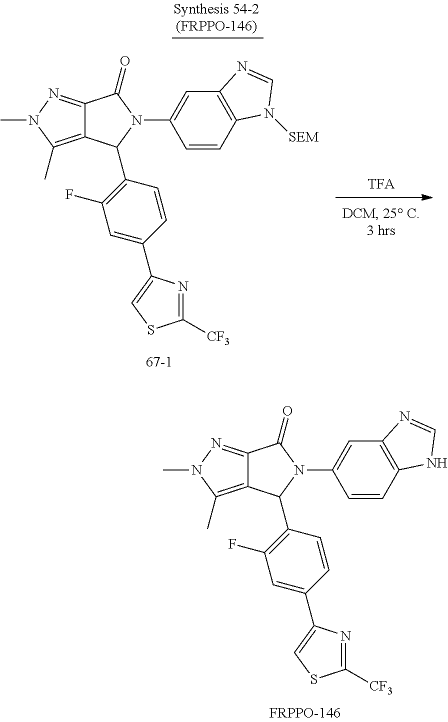

To a solution of compound 67-1 (60 mg, 93.4 μmol, 1.00 eq) in DCM (2 mL) was added TFA (3.08 g, 27.0 mmol, 2 mL, 289 eq). The mixture was stirred at 20° C. for 2 hrs. The reaction mixture was concentrated under reduced pressure to give a residue. The residue was purified by prep-HPLC (basic condition, column: Xtimate C18 150*25 mm*5 μm; mobile phase: 0.05% ammonia hydroxide, A-water, B-ACN; gradient: 32%-62% B, 10 min) to give FRPPO-146 (7.01 mg, 13.5 μmol, 14.5% yield, 98.7% purity) as yellow solid.

LCMS: RT=0.930 min, m/z=513.0 (M+1)⁺.

HPLC: RT=2.309 min.

¹H NMR: 400 MHz MeOD δ 8.20 (s, 1H), 8.14 (s, 1H), 7.71-7.70 (m, 3H), 7.68-7.65 (m, 1H), 7.39-7.38 (m, 1H), 7.30-7.26 (s, 1H), 6.60 (s, 1H), 3.94 (s, 3H), 2.22 (s, 3H).

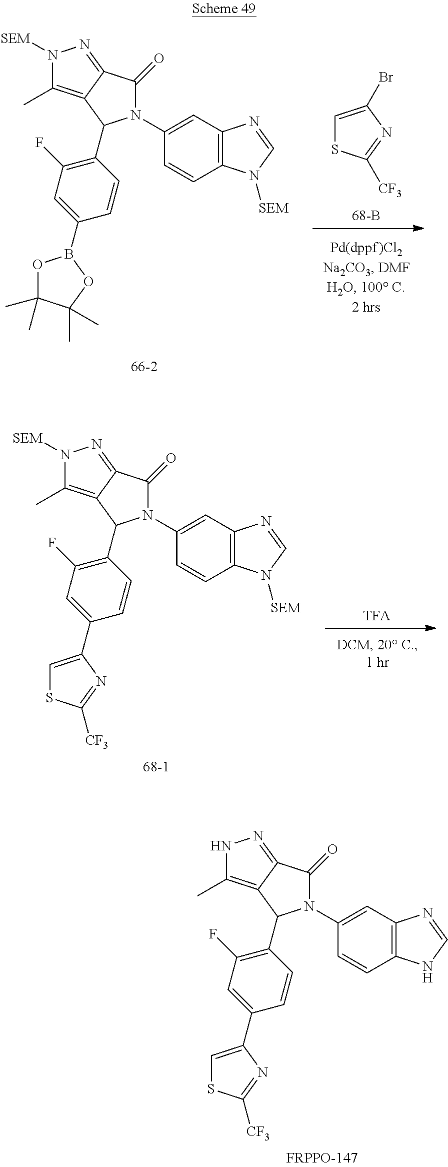 [SMILES: Cc1c2c(nn1COCC[Si](C)(C)C)C(=O)N(c1ccc3c(c1)ncn3COCC[Si](C)(C)C)C2c1ccc(B2OC(C)(C)C(C)(C)O2)cc1F ; FC(F)(F)c1nc(Br)cs1 ; Cc1c2c(nn1COCC[Si](C)(C)C)C(=O)N(c1ccc3c(c1)ncn3COCC[Si](C)(C)C)C2c1ccc(-c2csc(C(F)(F)F)n2)cc1F ; Cc1[nH]nc2c1C(c1ccc(-c3csc(C(F)(F)F)n3)cc1F)N(c1ccc3[nH]cnc3c1)C2=O]

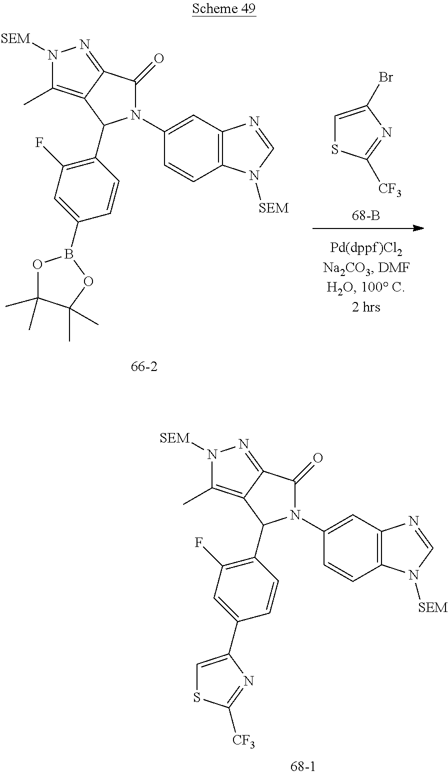 [SMILES: Cc1c2c(nn1COCC[Si](C)(C)C)C(=O)N(c1ccc3c(c1)ncn3COCC[Si](C)(C)C)C2c1ccc(B2OC(C)(C)C(C)(C)O2)cc1F ; FC(F)(F)c1nc(Br)cs1 ; Cc1c2c(nn1COCC[Si](C)(C)C)C(=O)N(c1ccc3c(c1)ncn3COCC[Si](C)(C)C)C2c1ccc(-c2csc(C(F)(F)F)n2)cc1F]

To a solution of compound 66-2 (Scheme 47 and Synthesis 53-2) (300 mg, 409 μmol, 1.00 eq) and compound 68-B (100 mg, 431 μmol, 1.05 eq) in DMF (6 mL) and H₂O (3 mL) was added Na₂CO₃ (130 mg, 1.23 mmol, 3.00 eq) and Pd(dppf)Cl₂ (60 mg, 82.0 μmol, 0.20 eq). The mixture was stirred at 100° C. for 2 hrs. The reaction mixture was diluted with water 30 mL and extracted with EtOAc (40 mL*3). The combined organic layers were washed with brine (50 mL*2), dried over Na₂SO₄, filtered and concentrated under reduced pressure to give a residue. The crude product was purified by reversed-phase HPLC (0.1% FA condition). The residue was purified by prep-HPLC (FA condition, column: Xtimate C18 150*25 mm*5 μm; mobile phase: 0.225% FA, A—water, B—ACN; gradient: 65%-95% B, 10 min) to give compound 68-1 (30 mg, 39.5 μmol, 9.67% yield) as yellow oil.

LCMS: RT=1.035 min, m/z=759.2 (M+1)⁺.

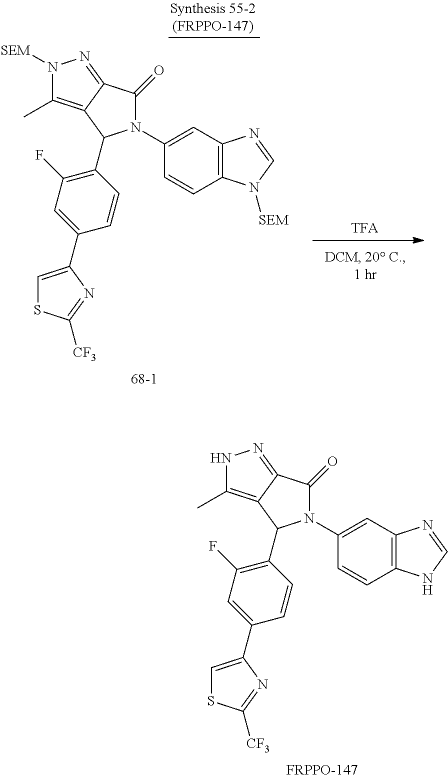

To a solution of compound 68-1 (30 mg, 39.5 μmol, 1.00 eq) in DCM (2 mL) was added TFA (1.54 g, 13.5 mmol, 1 mL, 342 eq). The mixture was stirred at 20° C. for 1 hr. The reaction mixture was concentrated under reduced pressure to give a residue. The residue was purified by prep-HPLC (basic condition, column: Xtimate C18 150*25 mm*5 μm; mobile phase: 0.05% ammonia hydroxide, A-water, B-ACN; gradient: 26%-56% B, 10 min) to give FRPPO-147 (4.45 mg, 8.86 μmol, 22.4% yield, 99.2% purity) as off-white solid.

LCMS: RT=0.937 min, m/z=499.3 (M+1)⁺.

HPLC: RT=2.847 min.

¹H NMR: 400 MHz MeOD δ 8.20 (s, 1H), 8.14 (s, 1H), 7.71-7.70 (m, 3H), 7.68-7.65 (m, 1H), 7.40-7.39 (m, 1H), 7.28-7.26 (s, 1H), 6.61 (s, 1H), 2.21-2.15 (s, 3H).

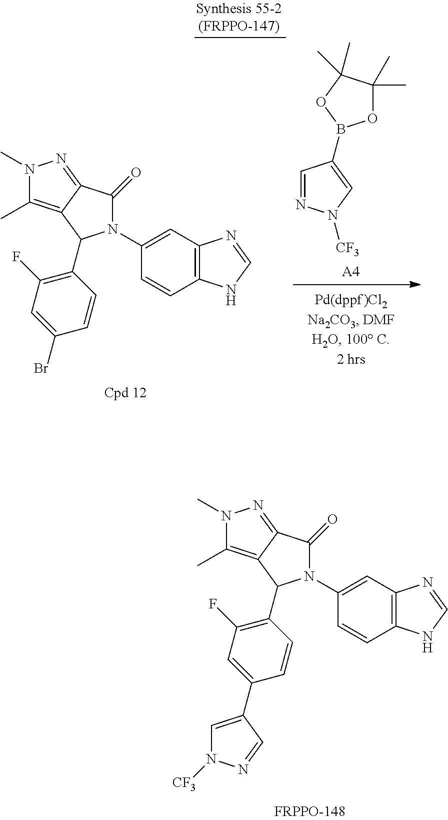

To a solution of compound Cpd 12 (Scheme 4 and Synthesis 5-2) (300 mg, 681 μmol, 1.00 eq) and compound A4 (540 mg, 2.06 mmol, 3.02 eq) in DMF (4 mL) and H₂O (1 mL) was added Pd(dppf)Cl₂ (100 mg, 137 μmol, 0.20 eq) and Na₂CO₃ (220 mg, 2.08 mmol, 3.05 eq). The mixture was stirred at 100° C. for 2 hr. The reaction mixture was filtered and concentrated under reduced pressure to give a residue. The residue was purified by prep-HPLC (FA condition; column: 3_Phenomenex Luna C18 75*30 mm*3 μm; mobile phase: 0.225% FA, A-water, B-ACN; gradient: 20%-40% B, 8 min) to give FRPPO-148 (71.88 mg, 140 μmol, 20.5% yield, 96.4% purity) as white solid.

LCMS: RT=0.773 min, m/z=496.1 (M+1)⁺.

HPLC: RT=1.637 min.

¹H NMR: 400 MHz CDCl₃ δ 7.97-7.89 (m, 3H), 7.54-7.53 (m, 1H), 7.16-7.13 (m, 1H), 7.12-7.08 (m, 3H), 6.46 (s, 1H), 3.95 (s, 3H), 2.21 (s, 3H).

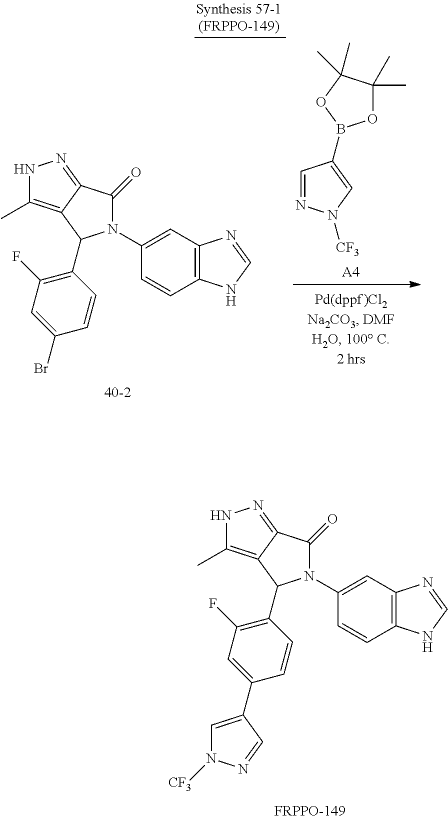

To a solution of compound 40-2 (Scheme 42 and Synthesis 46-2) (100 mg, 235 μmol, 1.00 eq) and compound A4 (184 mg, 702 μmol, 2.99 eq) in DMF (2 mL) and H₂O (0.2 mL) was added Pd(dppf)Cl₂ (35 mg, 47.8 μmol, 0.2 eq) and Na₂CO₃ (75 mg, 708 μmol, 3.02 eq). The mixture was stirred at 100° C. for 2 hrs. The mixture was filtrated and the filtrate was concentrated to give a residue. The residue was purified by prep-TLC (SiO₂, DCM MeOH=10:1) to get FRPPO-149 (DCM:MeOH=10:1, R_{f}=0.10) (19.39 mg, 39.4 μmol, 16.8% yield, 97.8% purity) as a gray solid.

LCMS: RT=0.900 min, m/z=482.4 (M+1)⁺.

HPLC: RT=2.294 min.

¹H NMR: 400 MHz DMSO-d6 δ 13.37 (s, 1H), 12.44 (s, 1H), 8.95 (s, 1H), 8.40 (s, 1H), 8.18-8.17 (m, 1H), 7.74-7.72 (m, 1H), 7.57-7.55 (m, 3H), 7.45-7.29 (m, 2H), 6.65-6.62 (m, 1H), 2.12 (s, 3H).

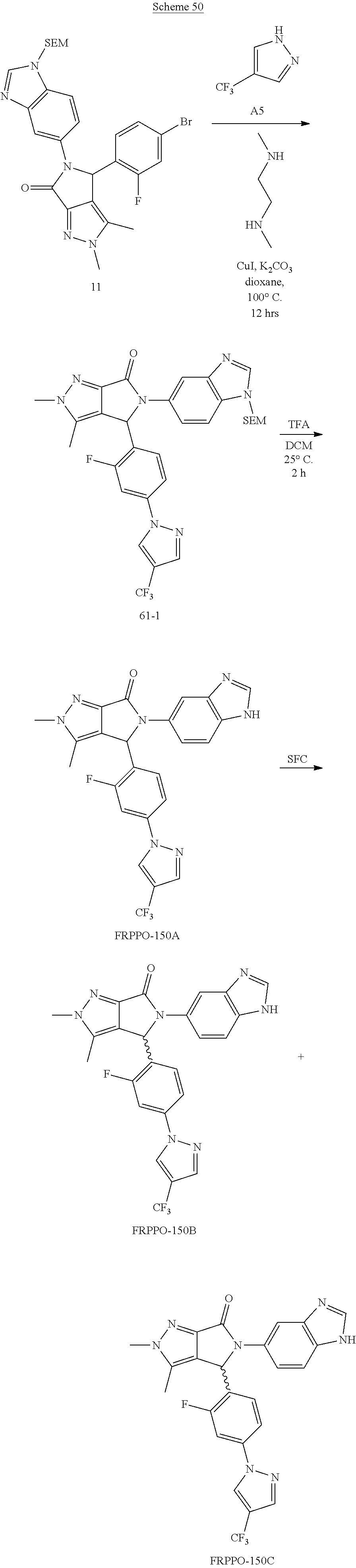

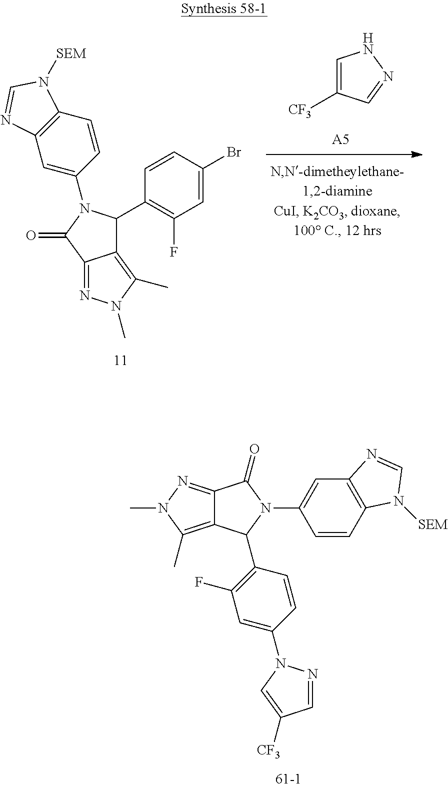

To a solution of compound 11 (Scheme 45 and Synthesis 50-1) (200 mg, 350 μmol, 1.00 eq) and compound A5 (100 mg, 734 μmol, 2.10 eq) in dioxane (5 mL) was added N,N′-dimethylethane-1,2-diamine (70 mg, 794 μmol, 85.47 μL, 2.27 eq) and CuI (67 mg, 351 μmol, 1.00 eq) and K₂CO₃ (100 mg, 723 μmol, 2.06 eq). The mixture was stirred at 100° C. for 12 hrs. The reaction mixture was partitioned between water 100 mL and Ethyl acetate 100 mL. The organic phase was separated, washed with brine 50 mL, dried over Na₂SO₄, filtered and concentrated under reduced pressure to give a residue. The residue was purified by prep-TLC (SiO₂, DCM:MeOH=10:1, product R_{f}=0.5) to give compound 61-1 (200 mg, 319 μmol, 91.1% yield) as a yellow oil, which was used in the next step directly.

LCMS: RT=1.110 min, m/z=626.5 (M+1)⁺.

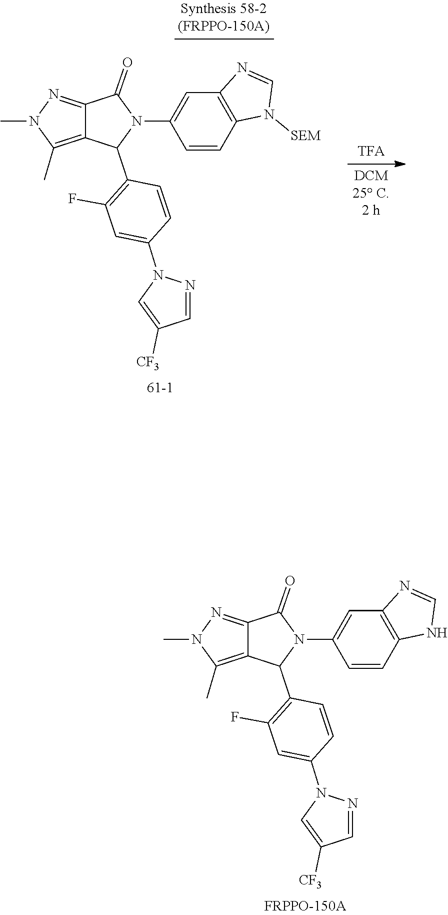 [SMILES: Cc1c2c(nn1C)C(=O)N(c1ccc3c(c1)ncn3COCC[Si](C)(C)C)C2c1ccc(-n2cc(C(F)(F)F)cn2)cc1F ; Cc1c2c(nn1C)C(=O)N(c1ccc3[nH]cnc3c1)C2c1ccc(-n2cc(C(F)(F)F)cn2)cc1F]

To a solution of compound 61-1 (200 mg, 319 μmol, 1.00 eq) in DCM (2 mL) was added TFA (3.08 g, 27.0 mmol, 2 mL, 84.5 eq). The mixture was stirred at 25° C. for 2 hrs. The reaction mixture was poured into water 20 mL and adjusted to pH=8-9 with NH₃.H₂O, and extracted with DCM 60 mL (30 mL*2), the organic phase was separated, washed with water 60 mL (30 mL*2) and brine 30 mL, dried over Na₂SO₄, filtered and concentrated under reduced pressure to give a residue. The residue was purified by prep-HPLC (basic condition, column: Waters Xbridge 150*25 mm*5 μm; mobile phase: 0.05% ammonia hydroxide, A—water, B—ACN; gradient: 26%-56% B, 10 min) to give FRPPO-150A (51.87 mg, 104 μmol, 32.7% yield, 100% purity) as a off-white solid.

LCMS: RT=0.926 min, m/z=496.4 (M+1)⁺.

HPLC: RT=1.946 min

¹H NMR: 400 MHz DMSO-d6 δ 12.4 (s, 1H), 9.11 (s, 1H), 8.18-8.17 (m, 2H), 7.73-7.70 (m, 2H), 7.64-7.62 (m, 1H), 7.47-7.45 (m, 1H), 6.67 (s, 1H), 3.88 (s, 3H), 2.15 (s, 3H).

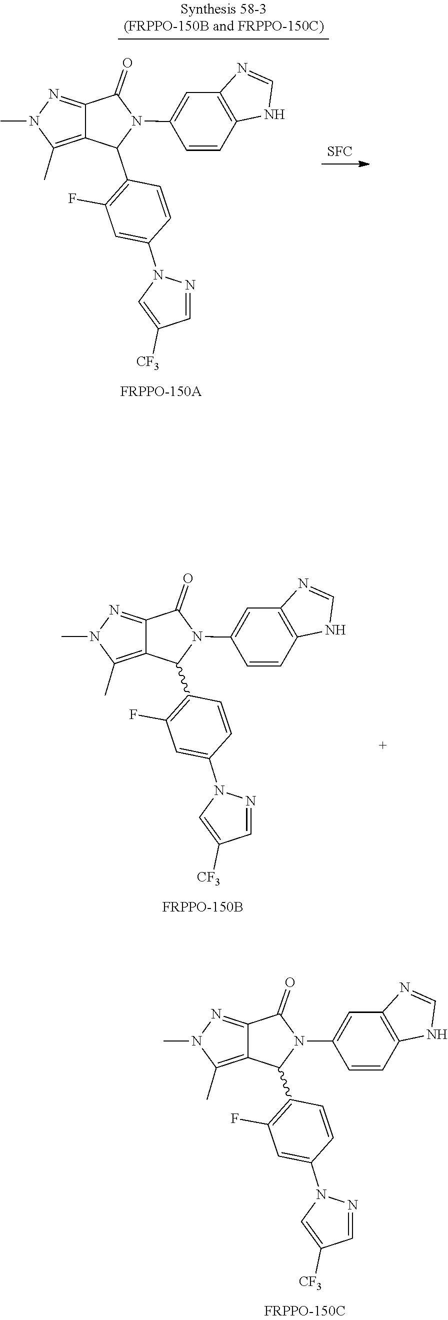

FRPPO-150A (51.87 mg, 104 μmol, 1.00 eq) was separated by chiral SFC chromatography. Column: Chiralpak AS 250×30 mm ID, 10 micron particle size. Mobile phase: 60% A—C02; 40% B—MeOH (0.1% NH₃.H₂O) at 70 g/min, 25° C., to give:

FRPPO-150B (20.09 mg, 40.5 μmol, 38.7% yield) as an off-white solid.

LCMS: RT=0.940 min, m/z=496.3 (M+1)⁺.

HPLC: RT=1.956 min

SFC: RT=1.333 min

¹H NMR: 400 MHz DMSO-d6 δ 12.4 (s, 1H), 9.12 (s, 1H), 8.18-8.17 (m, 2H), 7.75-7.71 (m, 2H), 7.65-7.62 (m, 1H), 7.46-7.44 (m, 2H), 7.43-7.31 (m, 1H), 6.68-6.66 (m, 1H), 3.88 (s, 3H), 2.15 (s, 3H).

FRPPO-150C (20.42 mg, 41.2 μmol, 39.3% yield) as an off-white solid.

LCMS: RT=0.938 min, m/z=496.3 (M+1)⁺.

HPLC: RT=1.959 min.

SFC: RT=1.670 min.

¹H NMR: 400 MHz DMSO-d6 δ 12.4 (s, 1H), 9.12 (s, 1H), 8.18-8.17 (m, 2H), 7.75-7.71 (m, 2H), 7.65-7.62 (m, 1H), 7.46-7.44 (m, 2H), 7.43-7.31 (m, 1H), 6.68-6.66 (m, 1H), 3.88 (s, 3H), 2.15 (s, 3H).

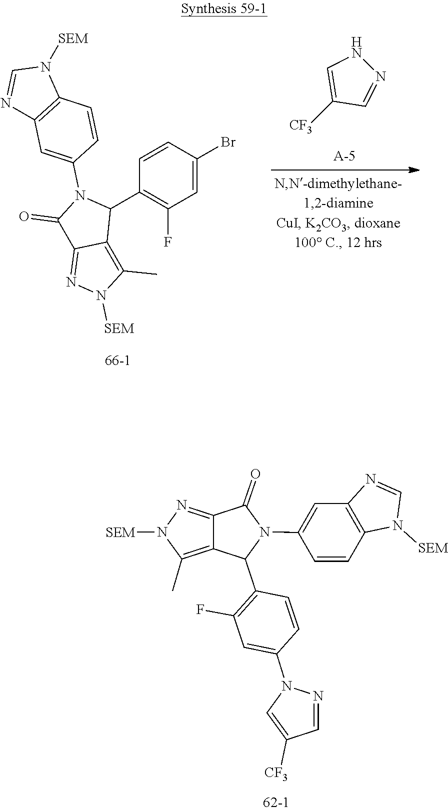

A mixture of compound 66-1 (Scheme 47 and Synthesis 53-1) (200 mg, 289 μmol, 1.00 eq), compound A5 (80.0 mg, 588 μmol, 2.03 eq), CuI (60.0 mg, 315 μmol, 1.09 eq), N,N′-dimethylethylenediamine (50.0 mg, 567 μmol, 61.0 μL, 1.96 eq), K₂CO₃ (80.0 mg, 579 μmol, 2.00 eq) and dioxane (5 mL) was stirred at 100° C. for 18 hrs. The mixture was filtrated and the filtrate was concentrated to give a residue. The crude product was purified by reversed-phase HPLC (0.1% FA condition in acetion) to give compound 62-1 (130 mg, crude) as a yellow solid and used in the next step directly.

LCMS: RT=1.091 & 1.104 min, m/z=742.3 (M+1)⁺.

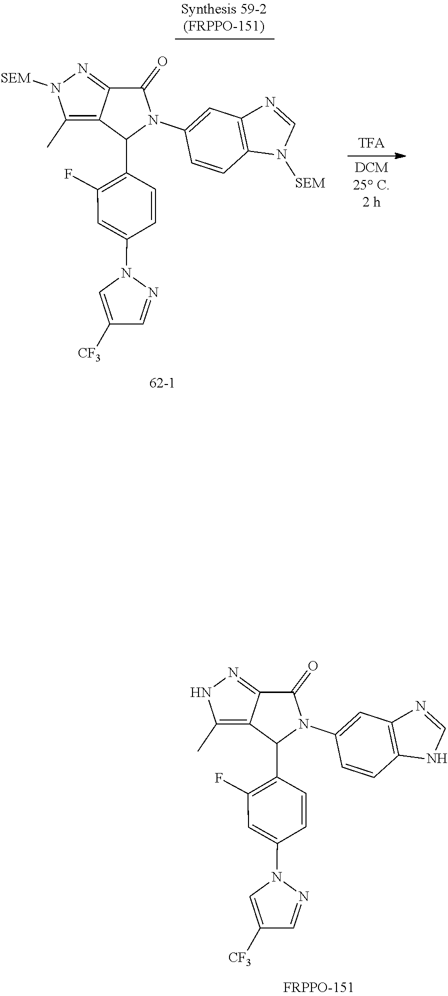 [SMILES: Cc1c2c(nn1COCC[Si](C)(C)C)C(=O)N(c1ccc3c(c1)ncn3COCC[Si](C)(C)C)C2c1ccc(-n2cc(C(F)(F)F)cn2)cc1F ; Cc1[nH]nc2c1C(c1ccc(-n3cc(C(F)(F)F)cn3)cc1F)N(c1ccc3[nH]cnc3c1)C2=O]

To a solution of 62-1 (100 mg, 135 μmol, 1.00 eq) in DCM (4 mL) was added TFA (3.08 g, 27.0 mmol, 2 mL, 200 eq) and the mixture was stirred at 25° C. for 12 hrs. The mixture was diluted with DCM (20 mL) and adjusted to pH=8 with Na₂CO₃ solution, extracted with DCM (10 mL*2). The combined organic layers were dried over anhydrous sodium sulfate, filtrated and concentrated to give a residue. The residue was purified by prep-HPLC (basic condition, column: Waters Xbridge 150*25 mm*5 μm mobile phase: 0.05% ammonia hydroxide, A—water, B—ACN; gradient: 23%-50% B, 10 min) to give compound FRPPO-151 (14.36 mg, 29.8 μmol, 22.1% yield, 99.9% purity) as a off-white solid.

LCMS: RT=0.898 min, m/z=482.2 (M+1)⁺.

HPLC: RT=2.466 min.

¹H NMR: 400 MHz DMSO-d6 δ 9.12 (s, 1H), 8.18-8.17 (m, 2H), 7.74-7.72 (m, 2H), 7.64-7.62 (m, 1H), 7.62-7.52 (m, 1H), 7.51-7.50 (m, 1H), 7.43-7.36 (m, 1H), 6.67 (s, 1H), 2.12 (s, 3H).

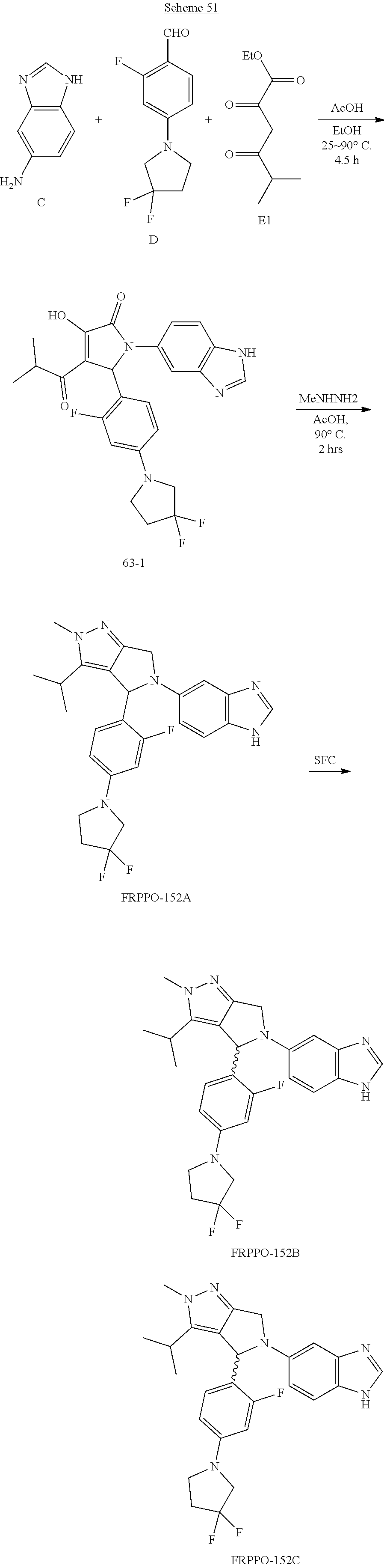

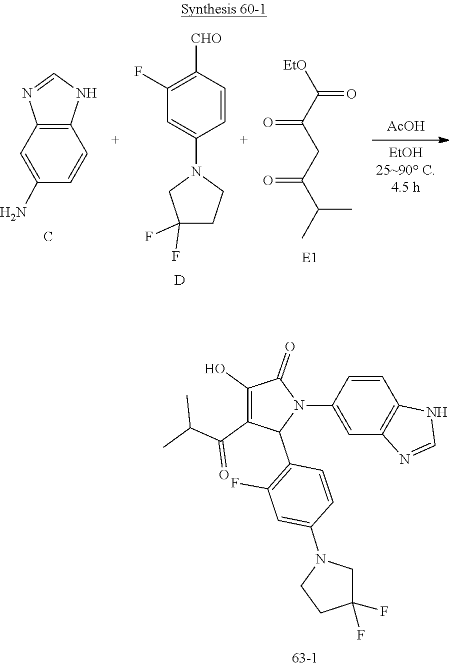

To a solution of compound D (861 mg, 3.76 mmol, 1.00 eq) and compound C (500 mg, 3.76 mmol, 1.00 eq) in EtOH (5 mL) was added AcOH (210 mg, 3.50 mmol, 0.2 mL, 0.9 eq). The mixture was stirred at 25° C. for 0.5 hr, then compound E1 (700 mg, 3.76 mmol, 1.00 eq) was added, the mixture was stirred at 90° C. for 4 hrs. The reaction mixture was filtered to give compound 63-1 (200 mg, 412 μmol, 10.9% yield) as a yellow solid.

¹H NMR: 400 MHz DMSO-d6 δ 8.21-8.19 (m, 1H), 7.67-7.66 (m, 1H), 7.49-7.48 (m, 1H), 7.34-7.30 (m, 1H), 6.22-6.10 (m, 2H), 5.96 (s, 1H), 3.59-3.53 (m, 2H), 3.38-3.36 (m, 3H), 2.45-2.42 (m, 2H), 0.99-0.96 (d, J=6.8 Hz, 3H), 0.94-0.92 (d, J=6.8 Hz, 3H).

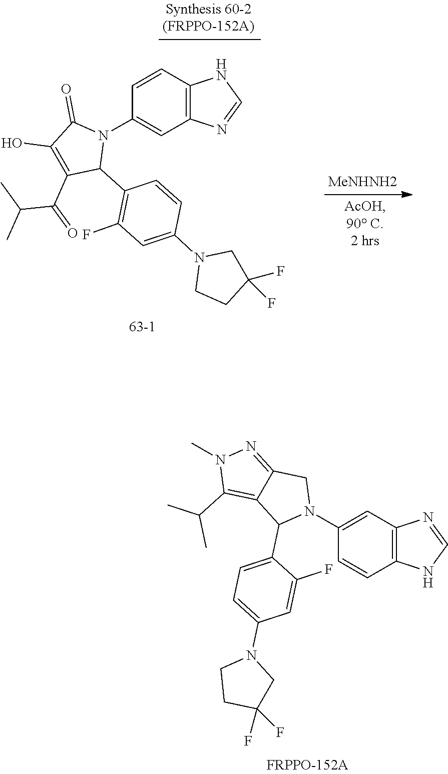 [SMILES: "MeNHNH2" ; CC(C)C(=O)C1=C(O)C(=O)N(c2ccc3[nH]cnc3c2)C1c1ccc(N2CCC(F)(F)C2)cc1F ; CC(C)c1c2c(nn1C)CN(c1ccc3[nH]cnc3c1)C2c1ccc(N2CCC(F)(F)C2)cc1F]

To a solution of compound 63-1 (200 mg, 412 μmol, 1.00 eq) in AcOH (5 mL) was added methylhydrazine (50 mg, 434 μmol, 57.1 μL, 1.05 eq). The mixture was stirred at 90° C. for 2 hrs. The reaction mixture was concentrated under reduced pressure to remove AcOH. The residue was diluted with water 100 mL and adjusted to pH=8-9 with NH₃.H₂O, then extracted with Ethyl acetate 150 mL (50 mL*3). The combined organic layers were washed with brine 100 mL, dried over Na₂SO₄, filtered and concentrated under reduced pressure to give a residue. The residue was purified by prep-HPLC (basic condition column: Phenomenex Gemini NX-C18 75*30 mm*3 μm; mobile phase: 0.05% ammonia hydroxide, A-water, B-ACN; 27%-57% B %, 7.5 min) to give FRPPO-152A (59.77 mg, 119 μmol, 28.9% yield, 98.9% purity) as a white solid.

LCMS: RT=0.948 min, m/z=495.4 (M+1)⁺.

HPLC: RT=1.992 min

¹H NMR: 400 MHz DMSO-d6 δ 12.4 (s, 1H), 8.16 (s, 1H), 7.60-7.46 (m, 1H), 7.26-7.24 (m, 1H), 7.09-6.93 (m, 1H), 6.76-6.48 (m, 1H), 6.28 (s, 1H), 6.26-6.20 (m, 2H), 3.92 (s, 3H), 3.61-3.55 (m, 2H), 3.44-3.40 (m, 2H), 3.12-3.06 (m, 1H), 2.45-2.38 (m, 2H), 1.18-1.17 (d, J=6.8 Hz, 3H), 0.72-0.69 (d, J=7.2 Hz, 3H).

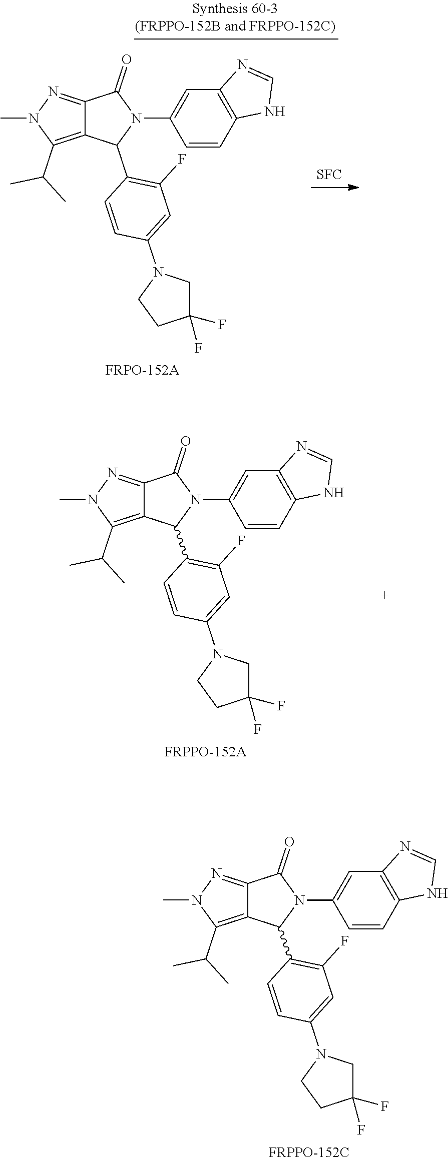 [SMILES: CC(C)c1c2c(nn1C)C(=O)N(c1ccc3[nH]cnc3c1)C2c1ccc(N2CCC(F)(F)C2)cc1F ; "SFC" ; CC(C)c1c2c(nn1C)C(=O)N(c1ccc3[nH]cnc3c1)C2c1ccc(N2CCC(F)(F)C2)cc1F ; CC(C)c1c2c(nn1C)C(=O)N(c1ccc3[nH]cnc3c1)C2c1ccc(N2CCC(F)(F)C2)cc1F]

FRPPO-152A (59.77 mg, 119 μmol, 1.00 eq) was separated by chiral SFC chromatography. Column: Chiralpak OD 250×30 mm ID, 10 micron particle size. Mobile phase: 50% A—C02; 50% B—MeOH (0.1% NH₃.H₂O) at 70 g/min, 25° C., to give: FRPPO-152B (19.33 mg, 38.6 μmol, 32.3% yield, 98.8% purity) as a light yellow solid

LCMS: RT=0.946 min, m/z=495.3 (M+1)⁺.

HPLC: RT=1.985 min.

SFC: RT=1.818 min.

¹H NMR: 400 MHz DMSO-d6 δ 12.4 (s, 1H), 8.17 (s, 1H), 7.62-7.52 (m, 1H), 7.42-7.40 (m, 1H), 7.29-7.21 (m, 1H), 6.49-6.46 (m, 1H), 6.28-6.26 (m, 1H), 6.24-6.21 (m, 2H), 3.92 (s, 3H), 3.61-3.55 (m, 2H), 3.37-3.33 (m, 2H), 3.13-3.09 (m, 1H), 2.47-2.42 (m, 2H), 1.18-1.17 (d, J=6.8 Hz, 3H), 0.72-0.70 (d, J=7.2 Hz, 3H).

FRPPO-152C (12.57 mg, 25.4 μmol, 21.2% yield, 100% purity) as a light yellow solid.

LCMS: RT=0.950 min, m/z=495.3 (M+1)⁺.

HPLC: RT=1.981 min.

SFC: RT=2.289 min.

¹H NMR: 400 MHz DMSO-d6 δ 12.4 (s, 1H), 8.17 (s, 1H), 7.62-7.52 (m, 1H), 7.42-7.40 (m, 1H), 7.29-7.21 (m, 1H), 6.49-6.46 (m, 1H), 6.28-6.26 (m, 1H), 6.24-6.21 (m, 2H), 3.92 (s, 3H), 3.61-3.55 (m, 2H), 3.37-3.34 (m, 2H), 3.11-3.07 (m, 1H), 2.47-2.42 (m, 2H), 1.19-1.17 (d, J=6.8 Hz, 3H), 0.72-0.70 (d, J=7.2 Hz, 3H).

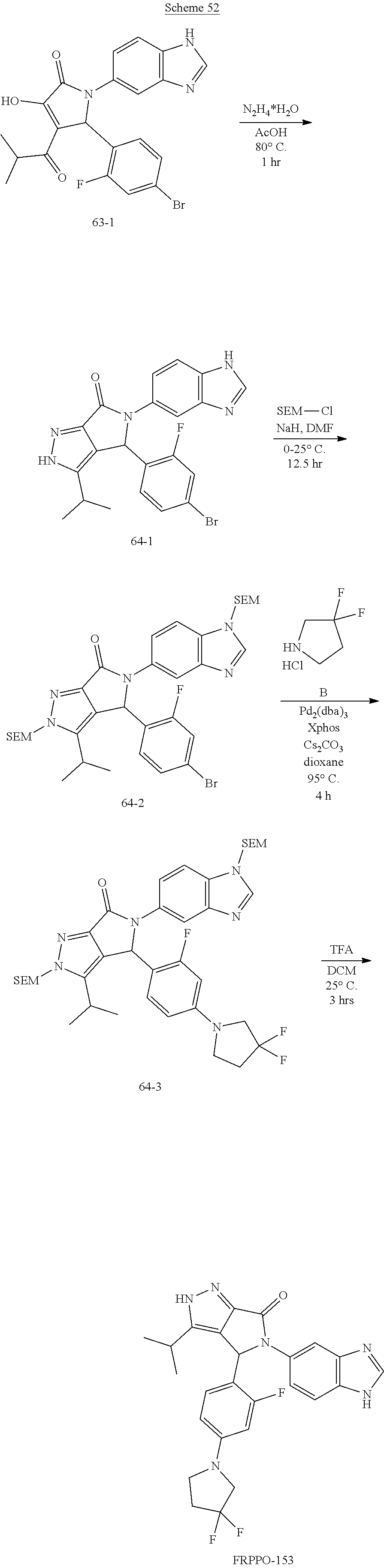

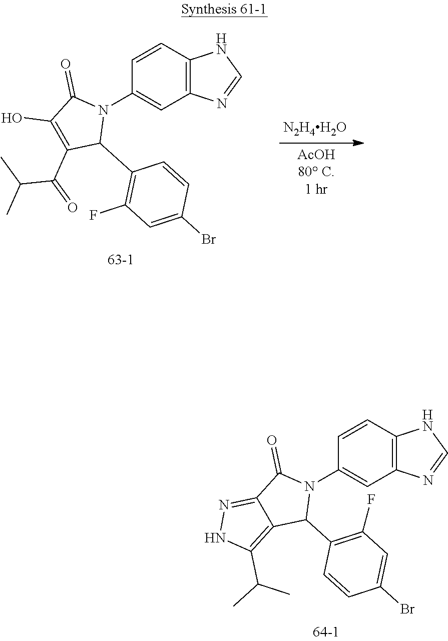 [SMILES: CC(C)C(=O)C1=C(O)C(=O)N(c2ccc3[nH]cnc3c2)C1c1ccc(Br)cc1F ; "N2H4·H2O" ; CC(C)c1[nH]nc2c1C(c1ccc(Br)cc1F)N(c1ccc3[nH]cnc3c1)C2=O]

To a solution of compound 63-1 (1.00 g, 2.18 mmol, 1.00 eq) in AcOH (6 mL) was added N₂H₄.H₂O (300 mg, 5.87 mmol, 291 μL, 2.69 eq). The mixture was stirred at 80° C. for 1 hr. The reaction mixture was quenched by addition water 10 mL at 25° C., and then concentrated under reduced pressure to remove AcOH, poured into water (20 mL), filtered and concentrated under reduced pressure to give 64-1 (2.00 g, crude) as yellow oil.

¹H NMR: 400 MHz DMSO-d6 δ 8.19 (s, 1H), 7.66-7.65 (m, 1H), 7.51-7.49 (m, 1H), 7.45-7.42 (m, 1H), 7.29-7.26 (m, 3H), 6.64 (s, 1H), 3.46-3.41 (m, 1H), 1.09-1.08 (m, 3H), 0.89-0.87 (m, 3H).

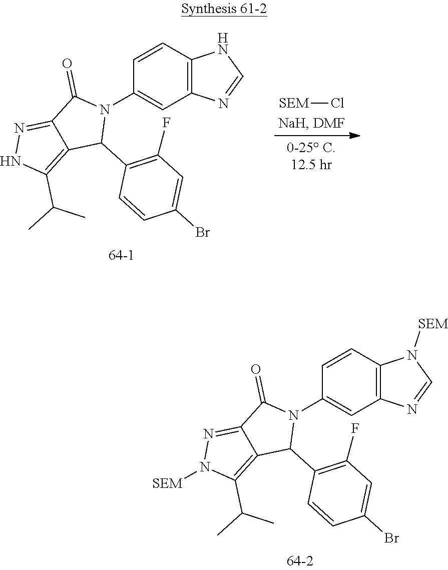 [SMILES: CC(C)c1[nH]nc2c1C(c1ccc(Br)cc1F)N(c1ccc3[nH]cnc3c1)C2=O ; C[Si](C)(C)CCOCCl ; CC(C)c1c2c(nn1COCC[Si](C)(C)C)C(=O)N(c1ccc3c(c1)ncn3COCC[Si](C)(C)C)C2c1ccc(Br)cc1F]

To a solution of compound 64-1 (2.00 g, 4.40 mmol, 1.00 eq) in DMF (10 mL) was added NaH (528 mg, 13.2 mmol, 60.0% purity, 3.00 eq) at 0° C. The mixture was stirred at 0° C. for 0.5 hr. Then SEM-Cl (2.20 g, 13.2 mmol, 2.34 mL, 3.00 eq) was added into the mixture. The mixture was stirred at 25° C. for 12 hrs. The mixture was quenched by addition NH₄Cl (10 mL) at 0° C., and extracted with EtOAc (20 mL*2). The combined organic layers were dried over anhydrous sodium sulfate, filtrated and concentrated to give a residue. The crude product was purified by reversed-phase HPLC (0.1% FA in acetonitrile) to get compound 64-2 (800 mg, 1.12 mmol, 25.4% yield) as yellow oil, which was used in the next step directly.

LCMS: EW20969-25-P1B2, RT=1.147&1.173 min. m/z=716.2 (M+3)⁺.

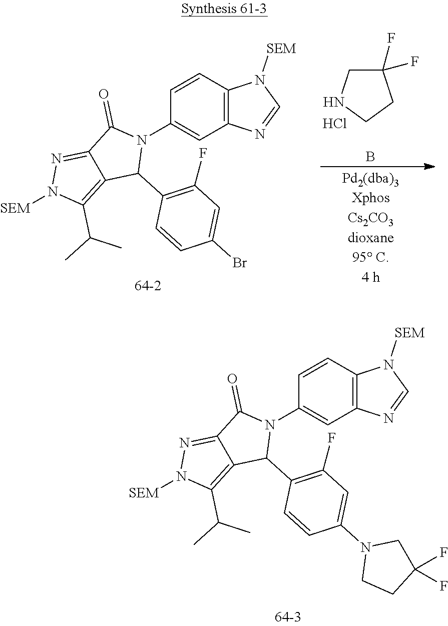

To solution of compound 64-2 (800 mg, 1.12 mmol, 1.00 eq) and compound B (192 mg, 1.34 mmol, 1.20 eq, HCl) in dioxane (5 mL) was added Pd₂(dba)₃ (51.0 mg, 55.6 μmol, 4.98e-2.00 eq), XPhos (53.0 mg, 111 μmol, 9.93e-2.00 eq) and Cs₂CO₃ (729 mg, 2.24 mmol, 2.00 eq). The mixture was stirred at 95° C. for 4 hrs. The mixture was filtered and concentrated under reduced pressure to give a residue. The crude product was purified by reversed-phase HPLC (0.1% FA condition) to get compound 64-3 (250 mg, 334 μmol, 29.9% yield, 99.2% purity) as yellow oil.

LCMS: RT=0.999 min, m/z=741.4 (M+1)⁺.

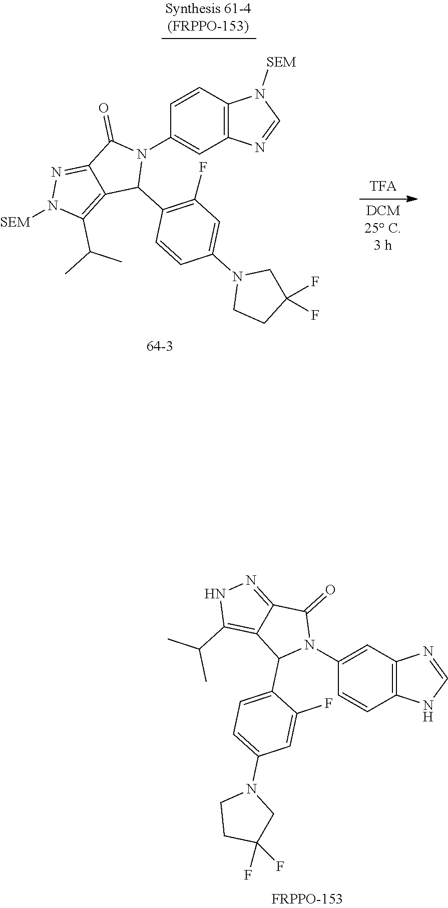 [SMILES: CC(C)c1c2c(nn1COCC[Si](C)(C)C)C(=O)N(c1ccc3c(c1)ncn3COCC[Si](C)(C)C)C2c1ccc(N2CCC(F)(F)C2)cc1F ; CC(C)c1[nH]nc2c1C(c1ccc(N3CCC(F)(F)C3)cc1F)N(c1ccc3[nH]cnc3c1)C2=O]

To a solution of compound 64-3 (200 mg, 269 μmol, 1.00 eq) in DCM (2 mL) was added TFA (1.54 g, 13.5 mmol, 1 mL, 50.0 eq). The mixture was stirred at 25° C. for 3 hrs. The mixture was diluted with DCM (20 mL) and adjusted to pH=8 with Na₂CO₃ solution, extracted with DCM (10 mL*2). The combined organic layers were dried over anhydrous sodium sulfate, filtrated and concentrated to give a residue. The residue was purified by prep-HPLC (neutral condition; column: Waters Xbridge 150*25 mm*5 μm; mobile phase: [water (10 mM NH₄HCO₃)-ACN]; B %: 27%-47%, 9 min) to get FRPPO-153 (9.73 mg, 19.9 μmol, 7.38% yield, 98.4% purity) as light yellow solid.

LCMS: RT=2.623 min, m/z=481.1 (M+1)⁺.

¹HNMR: 400 MHz DMSO-d6 δ 13.31-13.27 (m, 1H), 12.41 (s, 1H), 8.17 (s, 1H), 7.62-7.61 (m, 1H), 7.54-7.51 (m, 1H), 7.30-7.24 (m, 1H), 7.22-6.98 (m, 1H), 6.49-6.46 (m, 1H), 6.27-6.22 (m, 2H), 3.61-3.55 (m, 2H), 3.37-3.36 (m, 3H), 2.45-2.41 (m, 2H), 1.11-1.10 (m, 3H), 0.91-0.89 (m, 3H).

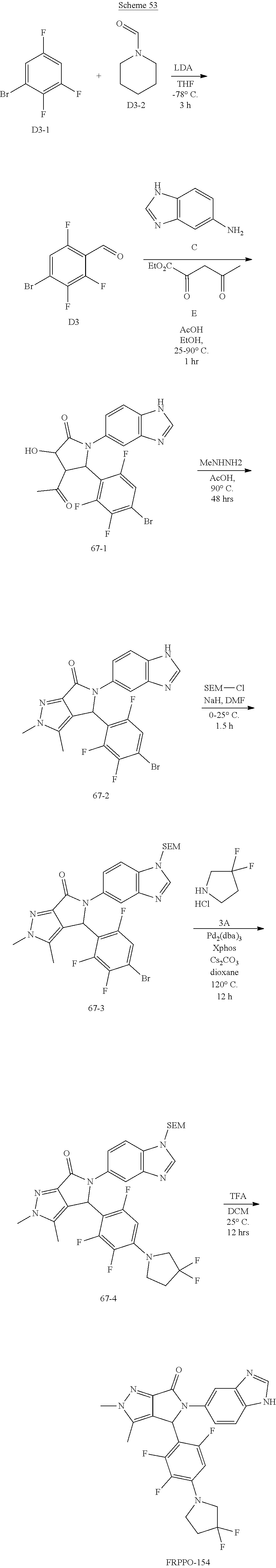

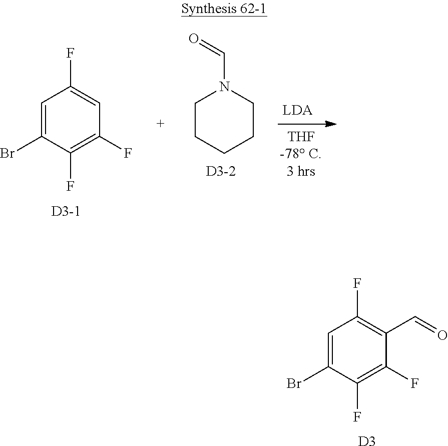

To a solution of compound D3-1 (500 mg, 2.37 mmol, 284 μL, 1.00 eq) in THF (5 mL) was added LDA (2 M, 1.90 mL, 1.60 eq) at −78° C. The mixture was stirred at −78° C. for 1 hr. Then compound D3-2 (322 mg, 2.85 mmol, 315 μL, 1.20 eq) was added into the mixture. The mixture was stirred at −78° C. for 2 hrs. TLC (petroleum ether:ethyl acetate=20:1) showed one new spot (R_{f}=0.50) was formed. The mixture was quenched by 3M HCl (pH=3) at −78° C., and then the mixture was extracted with Ethyl acetate (10 mL*2). The combined organic layers were dried over Na₂SO₄, filtered and concentrated under reduced pressure to give a residue. The residue was purified by prep-TLC (SiO₂, petroleum ether:ethyl acetate=20:1, product R_{f}=0.50) to compound D3 (400 mg, crude) as a yellow oil.

¹H NMR: 400 MHz CDCl₃ δ 10.3 (s, 1H), 7.30-7.25 (m, 1H).

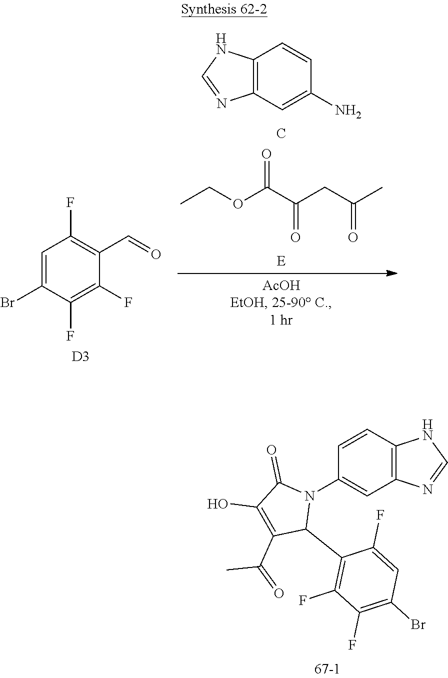

To a solution of compound D3 (3.59 g, 15.0 mmol, 1.00 eq) and compound C (2.00 g, 15.0 mmol, 1.00 eq) in EtOH (5 mL) was added AcOH (903 mg, 15.0 mmol, 860 μL, 1.00 eq). The mixture was stirred at 25° C. for 0.5 hr, then compound E (2.38 g, 15.0 mmol, 2.12 mL, 1.00 eq) was added, the mixture was stirred at 90° C. for 0.5 hr. The reaction mixture was filtered to give a residue. The crude product was triturated with Ethyl acetate (10 ml) at 25° C. for 30 min to give compound 67-1 (7.00 g, 15.0 mmol, 99.9% yield) as a yellow solid.

¹H NMR: 400 MHz DMSO-d6 δ 8.29 (s, 1H), 7.71-7.56 (m, 1H), 7.45-7.41 (m, 2H), 7.31-7.28 (m, 1H), 6.39-6.36 (m, 1H), 2.37 (s, 3H).

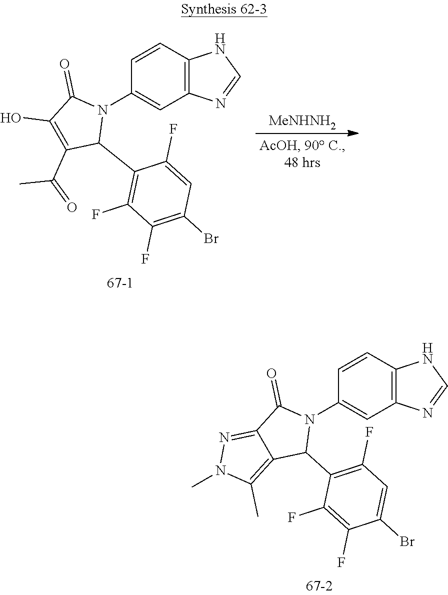 [SMILES: "MeNHNH2" ; CC(=O)C1=C(O)C(=O)N(c2ccc3[nH]cnc3c2)C1c1c(F)cc(Br)c(F)c1F ; Cc1c2c(nn1C)C(=O)N(c1ccc3[nH]cnc3c1)C2c1c(F)cc(Br)c(F)c1F]

To a solution of compound 67-1 (7.00 g, 15.0 mmol, 1.00 eq) in AcOH (30 mL) was added methylhydrazine (3.93 g, 34.1 mmol, 4.49 mL, 2.27 eq). The mixture was stirred at 90° C. for 48 hrs. The reaction mixture was concentrated under reduced pressure to remove AcOH. The residue was diluted with water 100 mL and adjusted to pH=8˜9 with NaHCO₃ (sat aq), then extracted with Ethyl acetate 450 mL (150 mL*3). The combined organic layers were washed with brine 200 mL, dried over Na₂SO₄, filtered and concentrated under reduced pressure to give a residue. The residue was purified by prep-HPLC (basic condition, column: Waters Xbridge BEH C18 250*50 mm*10 μm; mobile phase: 0.05% ammonia hydroxide, A-water, B-ACN; gradient: 25%-50% B %, 20 min) to give compound 67-2 (1.80 g, 3.78 mmol, 25.1% yield) as a yellow solid.

¹H NMR: 400 MHz DMSO-d6 δ 8.21 (s, 1H), 7.68-7.67 (m, 1H), 7.55-7.53 (m, 2H), 7.29-7.26 (m, 1H), 6.78 (s, 1H), 3.89 (s, 3H), 2.17 (s, 3H).

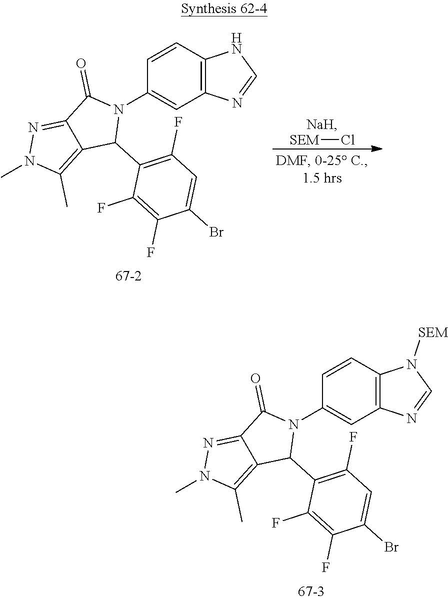 [SMILES: Cc1c2c(nn1C)C(=O)N(c1ccc3[nH]cnc3c1)C2c1c(F)cc(Br)c(F)c1F ; C[Si](C)(C)CCOCCl ; Cc1c2c(nn1C)C(=O)N(c1ccc3c(c1)ncn3COCC[Si](C)(C)C)C2c1c(F)cc(Br)c(F)c1F]

To a solution of compound 67-2 (1.00 g, 2.10 mmol, 1.00 eq) in DMF (10 mL) was added NaH (90 mg, 2.25 mmol, 60% purity, 1.07 eq) at 0° C., the mixture was stirred at 0° C. for 0.5 hr. then SEM-Cl (360 mg, 2.16 mmol, 382 μL, 1.03 eq) was added at 0° C., the mixture was stirred at 25° C. for 1 hr. The reaction mixture was partitioned between water 100 mL and Ethyl acetate 150 mL. The organic phase was separated, washed with water 100 mL (50 mL*2) and brine 100 mL, dried over Na₂SO₄, filtered and concentrated under reduced pressure to give a residue. The crude product was purified by reversed-phase HPLC (0.1% NH₃.H₂O in acetonitrile) to give compound 67-3 (1.20 g, 1.96 mmol, 93.4% yield, 99.2% purity) as a yellow solid.

LCMS: RT=1.083 min, m/z=606.4 (M+1)⁺.

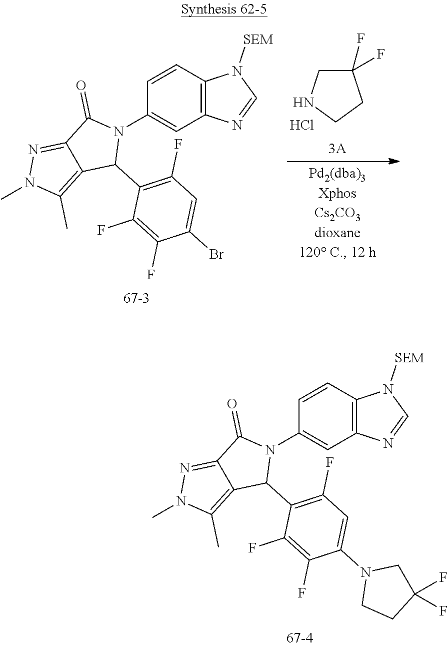 [SMILES: Cc1c2c(nn1C)C(=O)N(c1ccc3c(c1)ncn3COCC[Si](C)(C)C)C2c1c(F)cc(Br)c(F)c1F ; Cl ; FC1(F)CCNC1 ; Cc1c2c(nn1C)C(=O)N(c1ccc3c(c1)ncn3COCC[Si](C)(C)C)C2c1c(F)cc(N2CCC(F)(F)C2)c(F)c1F]

To a solution of compound 67-3 (100 mg, 163.56 μmol, 1.00 eq) and compound B (25 mg, 174 μmol, 1.06 eq, HCl) in dioxane (10 mL) was added Cs₂CO₃ (110 mg, 337 μmol, 2.06 eq) and Xantphos (10 mg, 17.2 μmol, 0.10 eq) and Pd₂(dba)₃ (15 mg, 16.3 μmol, 0.10 eq). The mixture was stirred at 120° C. for 12 hrs. The reaction mixture was partitioned between water 100 mL and Ethyl acetate 150 mL. The organic phase was separated, washed with water 100 mL (50 mL*2) and brine 100 mL, dried over Na₂SO₄, filtered and concentrated under reduced pressure to give a residue. The crude product was purified by reversed-phase HPLC (0.1% NH₃.H₂O in acetonitrile) to give compound 67-4 (50 mg, 79.0 μmol, 48.3% yield) as a yellow solid, which was confirmed by next step directly.

LCMS: RT=1.086 min, m/z=633.5 (M+1)⁺.

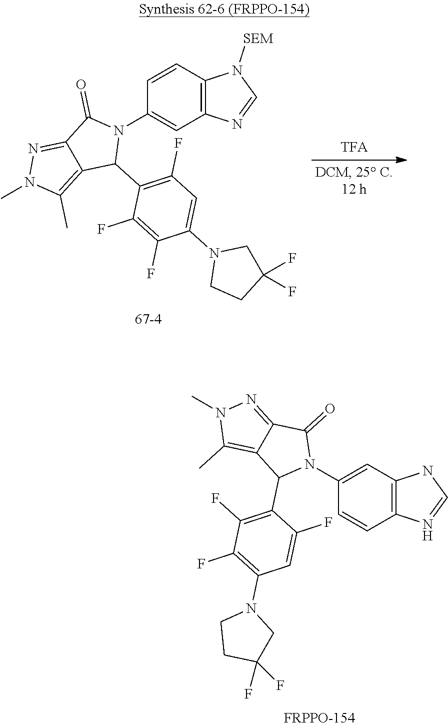 [SMILES: Cc1c2c(nn1C)C(=O)N(c1ccc3c(c1)ncn3COCC[Si](C)(C)C)C2c1c(F)cc(N2CCC(F)(F)C2)c(F)c1F ; Cc1c2c(nn1C)C(=O)N(c1ccc3nc[nH]c3c1)C2c1c(F)cc(N2CCC(F)(F)C2)c(F)c1F]

To a solution of compound 67-4 (50 mg, 79.0 μmol, 1.00 eq) in DCM (2 mL) was added TFA (3.08 g, 27.0 mmol, 2 mL, 341 eq). The mixture was stirred at 25° C. for 12 hrs. LCMS showed compound 67-4 was consumed, and desired m/z was detected. The reaction mixture was poured into water 100 mL and adjusted to pH=8-9 with NH₃.H₂O, then extracted with DCM 200 mL (100 mL*2). The combined organic layers were washed with brine 100 mL, dried over Na₂SO₄, filtered and concentrated under reduced pressure to give a residue. The residue was purified by prep-HPLC (basic condition column: Waters Xbridge 150*25 mm*5 μm; mobile phase: 0.05% ammonia hydroxide, A-water, B-ACN; gradient: 26%-56% B %, 10 min) to give FRPPO-154 (26.81 mg, 53.1 μmol, 67.2% yield, 99.6% purity) as a white solid.

LCMS: RT=0.936 min, m/z=503.4 (M+1)⁺.

HPLC: RT=1.891 min.

¹H NMR: 400 MHz DMSO-d6 δ 8.19 (s, 1H), 7.64-7.63 (m, 1H), 7.54-7.52 (m, 1H), 7.28-7.25 (m, 1H), 6.60 (s, 1H), 6.36 (s, 1H), 3.88 (s, 3H), 3.79-3.70 (m, 2H), 3.52-3.49 (m, 2H), 2.42-2.32 (m, 2H), 2.15 (s, 3H).

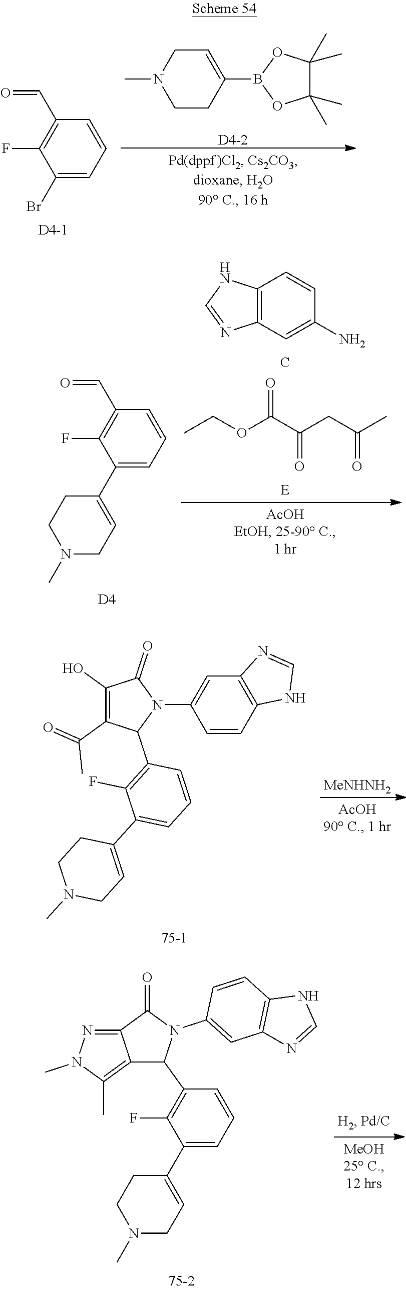

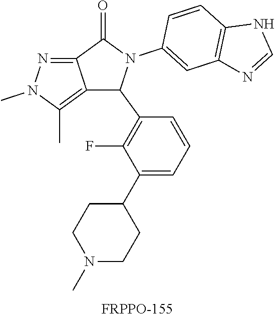 [SMILES: Cc1c2c(nn1C)C(=O)N(c1ccc3[nH]cnc3c1)C2c1cccc(C2CCN(C)CC2)c1F]

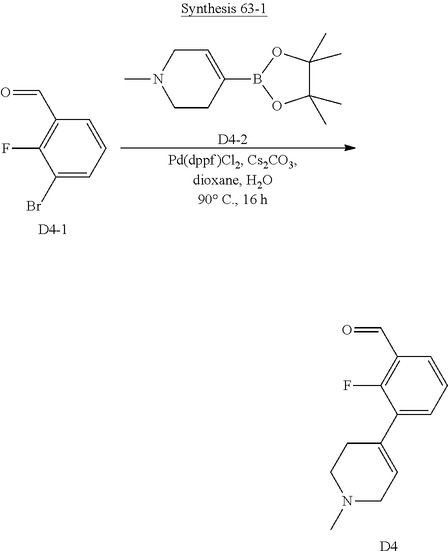

To a solution of compound D4-1 (3.00 g, 14.7 mmol, 1.00 eq) and compound D4-2 (3.30 g, 14.7 mmol, 1 eq) in dioxane (40 mL) and H₂O (5 mL) was added Pd(dppf)Cl₂ (540 mg, 738 μmol, 0.05 eq) and Cs₂CO₃ (15.0 g, 46.0 mmol, 3.12 eq). The mixture was stirred at 90° C. for 16 hrs under N₂ atmosphere. The reaction mixture was partitioned between water 100 mL and Ethyl acetate 100 mL. The organic phase was separated, washed with brine 100 mL (50 mL*2), dried over Na₂SO₄, filtered and concentrated under reduced pressure to give a residue. The crude product was purified by reversed-phase HPLC (0.1% FA condition) to give compound D4 (3.30 g, crude) as a red oil.

¹H NMR: 400 MHz CDCl₃ δ 10.4 (s, 1H), 7.82-7.78 (m, 1H), 7.53-7.51 (m, 1H), 7.27-7.24 (m, 1H), 5.98-5.96 (m, 1H), 3.61-3.59 (m, 2H), 3.18-3.15 (m, 2H), 2.79-2.78 (m, 2H), 2.72 (s, 3H).

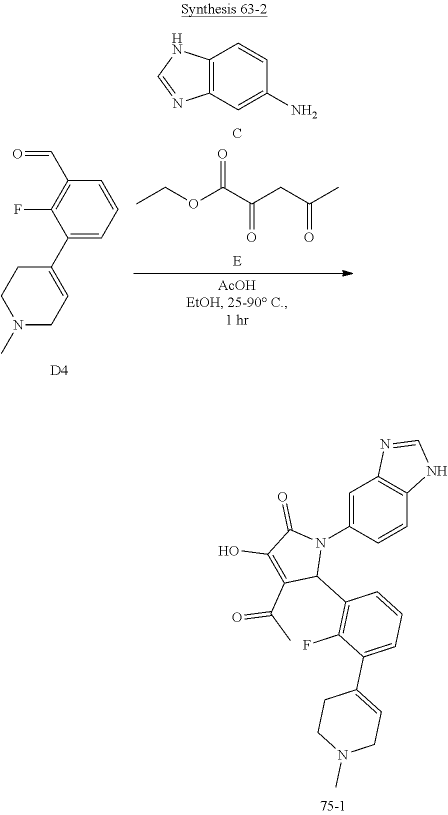

To a solution of compound D4 (3.30 g, 15.0 mmol, 1.00 eq) and compound C (2.01 g, 15.1 mmol, 1.00 eq) in EtOH (30 mL) was added AcOH (934 mg, 15.5 mmol, 890 μL, 1.03 eq). The mixture was stirred at 25° C. for 0.5 hr, then compound E (2.38 g, 15.0 mmol, 2.13 mL, 1.00 eq) was added, the mixture was stirred at 25° C. for 0.5 hr. The reaction mixture was filtered to give compound 75-1 (5.00 g, 11.2 mmol, 74.4% yield) as a green solid, and used to next step directly.

LCMS: RT=0.651 min, m/z=447.4 (M+1)⁺.

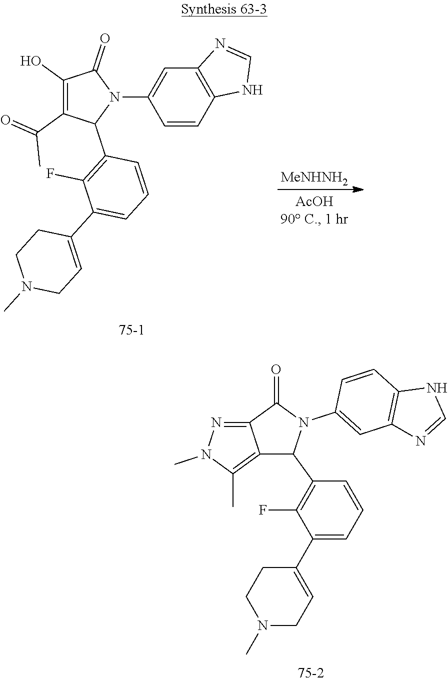 [SMILES: "MeNHNH2" ; CC(=O)C1=C(O)C(=O)N(c2ccc3[nH]cnc3c2)C1c1cccc(C2=CCN(C)CC2)c1F ; Cc1c2c(nn1C)C(=O)N(c1ccc3[nH]cnc3c1)C2c1cccc(C2=CCN(C)CC2)c1F]

To a solution of compound 75-1 (1.00 g, 2.24 mmol, 1.00 eq) in AcOH (10 mL) was added methylhydrazine (170 mg, 1.48 mmol, 194 μL, 0.66 eq). The mixture was stirred at 90° C. for 1 hr. The reaction mixture was concentrated under reduced pressure to remove AcOH. The residue was diluted with water 50 mL and adjusted to pH=8-9 with NH₃.H₂O, and extracted with DCM 90 mL (30 mL*3). The combined organic layers were washed with brine 40 mL, dried over Na₂SO₄, filtered and concentrated under reduced pressure to give a residue. The residue was purified by prep-HPLC (basic condition column: Phenomenex Gemini NX-C18 75*30 mm*3 μm; mobile phase: 0.05% ammonia hydroxide, A-water, B-ACN; gradient: 23%-40% B %, 10 min) to give compound 75-2 (250 mg, 547 μmol, 24.4% yield) as a yellow solid.

¹H NMR: 400 MHz DMSO-d6 δ 12.5 (s, 1H), 8.20 (s, 1H), 7.72-7.54 (m, 1H), 7.45-7.31 (m, 2H), 7.16-7.12 (m, 2H), 7.09-7.01 (m, 1H), 6.99-6.60 (m, 1H), 5.87 (s, 1H), 4.09 (s, 2H), 3.86 (s, 3H), 2.97-2.93 (m, 2H), 2.42-2.32 (m, 2H), 2.25 (s, 3H), 2.13 (s, 3H).

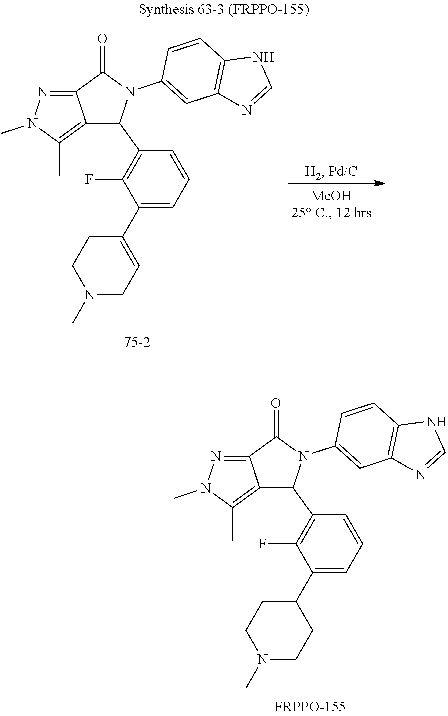 [SMILES: Cc1c2c(nn1C)C(=O)N(c1ccc3[nH]cnc3c1)C2c1cccc(C2=CCN(C)CC2)c1F ; "H2, Pd/C" ; Cc1c2c(nn1C)C(=O)N(c1ccc3[nH]cnc3c1)C2c1cccc(C2CCN(C)CC2)c1F]

To a solution of compound 75-2 (250 mg, 547 μmol, 1.00 eq) in MeOH (5 mL) was added Pd/C (10%, 0.1 g) under N₂ atmosphere. The suspension was degassed and purged with H₂ for 3 times. The mixture was stirred under H₂ (50 Psi) at 25° C. for 12 hrs. The reaction mixture was filtered through a pad of celite and concentrated under reduced pressure to give a residue. The residue was purified by prep-HPLC (basic condition column: Waters Xbridge 150*25 mm*5 μm; mobile phase: 0.05% ammonia hydroxide, A-water, B-ACN; gradient: 17%-47% B %, 10 min). The residue was purified by prep-TLC (SiO₂, DCM:MeOH=10:1, product R_{f}=0.4) to give FRPPO-155 (7.55 mg, 16.4 μmol, 2.99% yield, 99.6% purity) as a white solid.

LCMS: RT=0.905 min, m/z=459.4 (M+1)⁺.

HPLC: RT=1.590 min.

¹H NMR: 400 MHz CDCl₃ δ 7.95 (s, 1H), 7.78 (s, 1H), 7.53-7.51 (m, 1H), 7.09-7.05 (m, 1H), 6.93-6.91 (m, 1H), 6.89-6.88 (m, 2H), 6.43 (s, 1H), 3.92 (s, 3H), 3.03-3.01 (m, 2H), 2.79-2.76 (m, 1H), 2.37 (s, 3H), 2.18 (s, 3H), 2.14-2.13 (m, 2H), 2.11-1.82 (m, 2H), 1.27-1.23 (m, 2H).

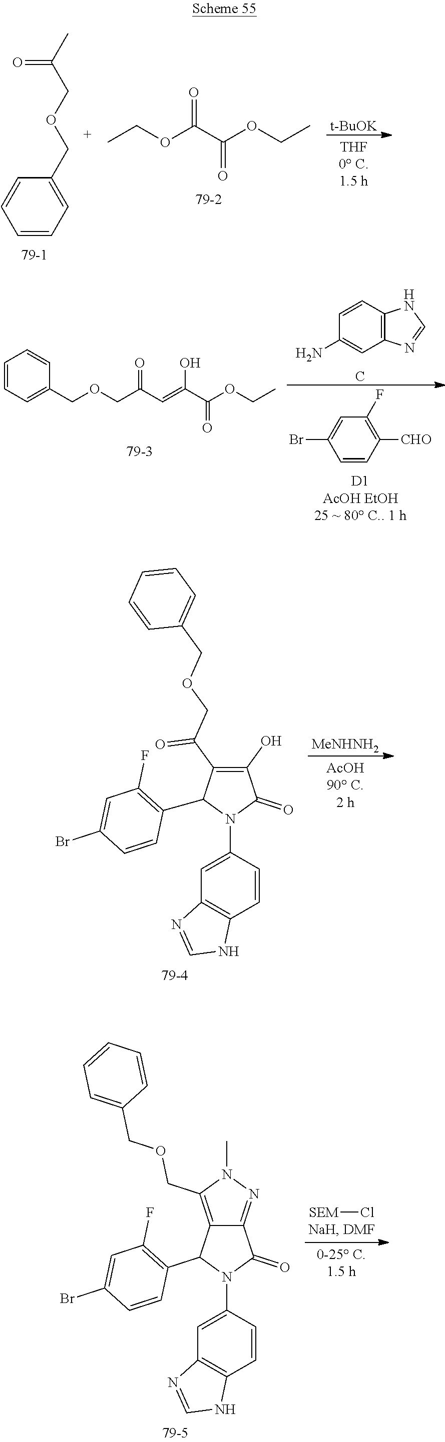

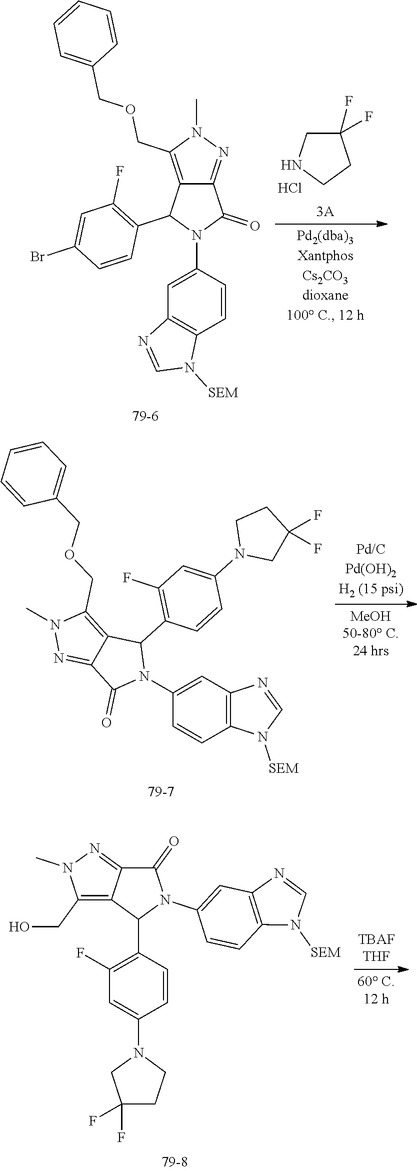 [SMILES: Cn1nc2c(c1COCc1ccccc1)C(c1ccc(Br)cc1F)N(c1ccc3c(c1)ncn3COCC[Si](C)(C)C)C2=O ; Cl ; FC1(F)CCNC1 ; Cn1nc2c(c1COCc1ccccc1)C(c1ccc(N3CCC(F)(F)C3)cc1F)N(c1ccc3c(c1)ncn3COCC[Si](C)(C)C)C2=O ; Cn1nc2c(c1CO)C(c1ccc(N3CCC(F)(F)C3)cc1F)N(c1ccc3c(c1)ncn3COCC[Si](C)(C)C)C2=O]

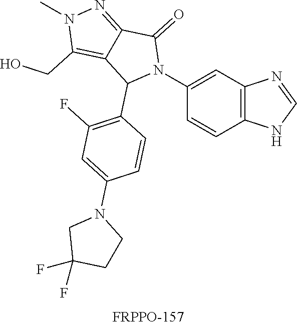 [SMILES: Cn1nc2c(c1CO)C(c1ccc(N3CCC(F)(F)C3)cc1F)N(c1ccc3[nH]cnc3c1)C2=O]

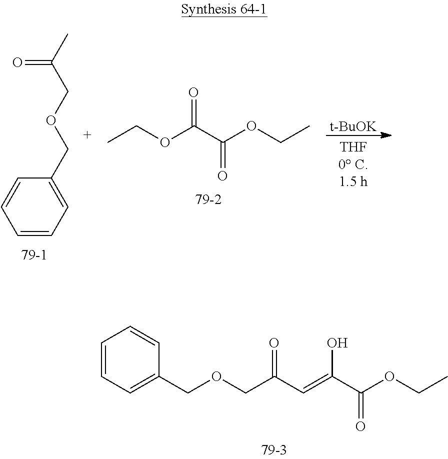

To a solution of t-BuOK (20.5 g, 182 mmol, 1.20 eq) in THF (150 mL) was added compound 79-2 (20.3 g, 139 mmol, 19 mL, 0.90 eq) at 0° C. The mixture was stirred at 0° C. for 0.5 hr, then compound 79-1 (25.0 g, 152 mmol, 1.00 eq) was added, the mixture was stirred at 0° C. for 1 hr. TLC (petroleum ether:ethyl acetate=3:1) showed compound 79-1 (R_{f}=0.7) remained, and new spot (R_{f}=0.2) formed. The reaction mixture was adjusted to pH=2 with HCl (1M) and extracted with Ethyl acetate 100 mL (50 mL*2). The combined organic layers were washed with brine 100 mL, dried over Na₂SO₄, filtered and concentrated under reduced pressure to give a residue. The residue was purified by flash silica gel chromatography (ISCO®; 20 g SepaFlash® Silica Flash Column, Eluent of 0-100% ethyl acetate:petroleum ether gradient @ 60 mL/min) to give compound 79-3 (5.90 g, 22.3 mmol, 14.6% yield) as a yellow oil.

¹H NMR: 400 MHz CDCl₃ δ 7.28-7.27 (m, 5H), 6.60 (s, 1H), 4.53 (s, 2H), 4.29-4.24 (m, 2H), 4.08 (s, 2H), 1.30-1.27 (m, 3H).

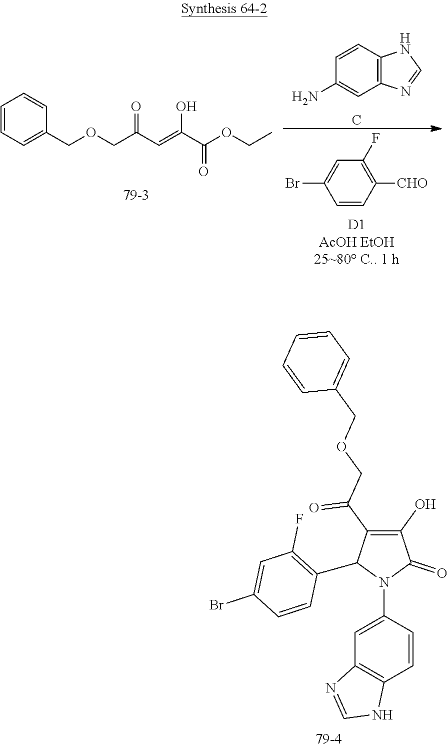

To a solution of compound C (3.01 g, 22.6 mmol, 1.01 eq) and compound D1 (4.57 g, 22.5 mmol, 1.01 eq) in EtOH (10 mL) was added AcOH (1.34 g, 22.3 mmol, 1.28 mL, 1.00 eq). The mixture was stirred at 25° C. for 0.5 hr, then compound 79-3 (5.90 g, 22.3 mmol, 1.00 eq) was added, the mixture was stirred at 80° C. for 1 hr. The reaction mixture was concentrated under reduced pressure to give a residue. The crude product was triturated with Ethyl acetate at 25° C. for 30 min to give compound 79-4 (5.10 g, 9.51 mmol, 42.5% yield) as a yellow solid.

¹H NMR: 400 MHz DMSO-d6 δ 8.26 (s, 1H), 7.71 (s, 1H), 7.53-7.51 (m, 1H), 7.34-7.29 (m, 7H), 7.28-7.23 (m, 2H), 6.24 (s, 1H), 4.64-4.55 (m, 2H), 4.50-4.48 (m, 2H).

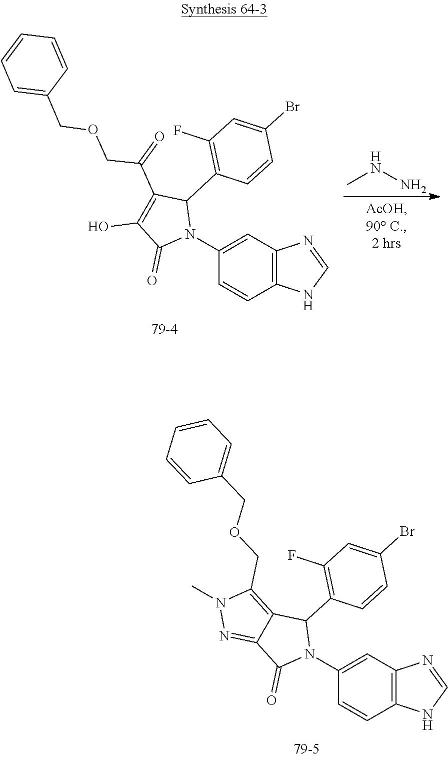

To a solution of compound 79-4 (5.10 g, 9.51 mmol, 1.00 eq) in AcOH (10 mL) was added methylhydrazine (7.76 g, 67.3 mmol, 8.87 mL, 7.09 eq). The mixture was stirred at 90° C. for 2 hrs. The reaction mixture was concentrated under reduced pressure to remove AcOH. The residue was diluted with water 50 mL and adjusted to pH=8-9 with NH₄OH, then extracted with Ethyl acetate 300 mL (100 mL*3). The combined organic layers were washed with brine 100 mL, dried over Na₂SO₄, filtered and concentrated under reduced pressure to give a residue. The residue was purified by prep-HPLC (basic condition column: Waters Xbridge BEH C18 250*50 mm*10 μm; mobile phase: 0.05% ammonia hydroxide, A-water, B-ACN; gradient: 30%-60% B %, 20 min) to give compound 79-5 (1.90 g, 3.43 mmol, 36.0% yield, 98.7% purity) as a yellow solid.

¹H NMR: 400 MHz DMSO-d6 δ 12.4 (s, 1H), 8.19 (s, 1H), 7.71-7.67 (m, 1H), 7.38-7.34 (m, 1H), 7.32-7.28 (m, 1H), 7.26-7.24 (m, 6H), 7.09-7.08 (m, 2H), 6.66-6.64 (m, 1H), 4.48 (s, 2H), 4.29-4.20 (m, 2H), 3.95 (s, 3H).

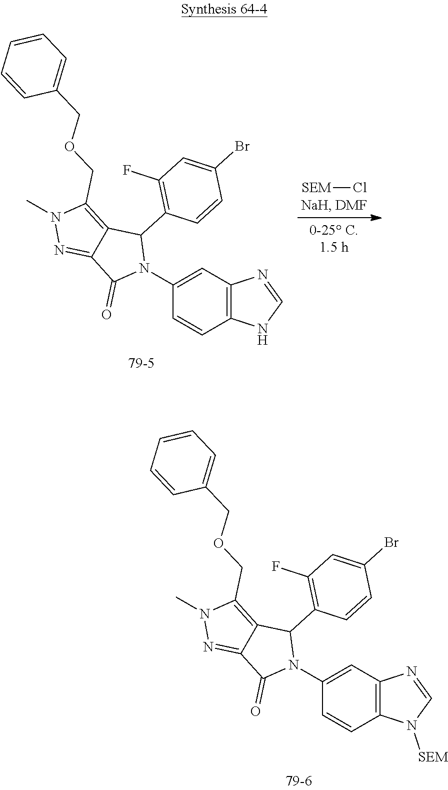 [SMILES: Cn1nc2c(c1COCc1ccccc1)C(c1ccc(Br)cc1F)N(c1ccc3[nH]cnc3c1)C2=O ; C[Si](C)(C)CCOCCl ; Cn1nc2c(c1COCc1ccccc1)C(c1ccc(Br)cc1F)N(c1ccc3c(c1)ncn3COCC[Si](C)(C)C)C2=O]

To a solution of compound 79-5 (1.80 g, 3.25 mmol, 1.00 eq) in DMF (180 mL) was added NaH (180 mg, 4.50 mmol, 60% purity, 1.38 eq) at 0° C. The mixture was stirred at 0° C. for 0.5 hr, and then SEM-Cl (600 mg, 3.60 mmol, 636 μL, 1.11 eq) was added at 0° C., the mixture was stirred at 25° C. for 1 hrs. The reaction mixture was partitioned between water 200 mL and Ethyl acetate 200 mL. The organic phase was separated, washed with water 600 mL (200 mL*3), dried over Na₂SO₄, filtered and concentrated under reduced pressure to give a residue. The crude product was purified by reversed-phase HPLC (0.1% NH₃.H₂O in acetonitrile) to give compound 79-6 (1.70 g, 2.51 mmol, 77.2% yield) as a yellow solid.

¹H NMR 400 MHz CDCl₃ δ 7.94-7.83 (m, 1H), 7.82-7.70 (m, 1H), 7.69-7.35 (m, 1H), 7.35-7.34 (m, 1H), 7.32-7.27 (m, 3H), 7.15-7.09 (m, 4H), 6.93-6.90 (m, 1H), 6.44-6.40 (m, 1H), 5.55-5.44 (m, 2H), 4.47-4.43 (m, 2H), 4.32-4.31 (m, 2H), 4.00 (s, 3H), 3.51-3.46 (m, 2H), 0.91-0.87 (m, 2H), 0.06-−0.02 (m, 9H).

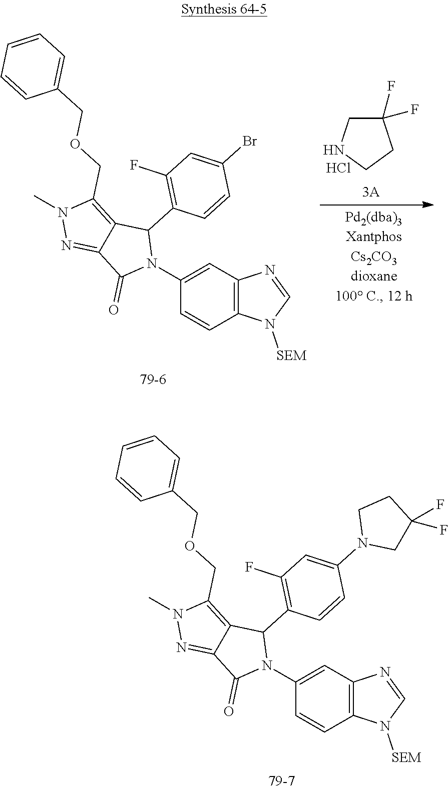 [SMILES: Cn1nc2c(c1COCc1ccccc1)C(c1ccc(Br)cc1F)N(c1ccc3c(c1)ncn3COCC[Si](C)(C)C)C2=O ; Cl ; FC1(F)CCNC1 ; Cn1nc2c(c1COCc1ccccc1)C(c1ccc(N3CCC(F)(F)C3)cc1F)N(c1ccc3c(c1)ncn3COCC[Si](C)(C)C)C2=O]

To a solution of compound 3A (352 mg, 2.45 mmol, 1.04 eq, HCl) and compound 79-6 (1.60 g, 2.36 mmol, 1.00 eq) in dioxane (15 mL) was added Xantphos (280 mg, 483.91 μmol, 0.20 eq) and Pd₂(dba)₃ (220 mg, 240 μmol, 0.10 eq) and Cs₂CO₃ (2.31 g, 7.09 mmol, 3.00 eq). The mixture was stirred at 100° C. for 12 hrs. The reaction mixture was partitioned between water 100 mL and Ethyl acetate 150 mL. The organic phase was separated, washed with brine 100 mL, dried over Na₂SO₄, filtered and concentrated under reduced pressure to give a residue. The crude product was purified by reversed-phase HPLC (0.1% NH₃.H₂O in acetonitrile) to give compound 79-7 (1.10 g, 1.47 mmol, 62.3% yield, 94.2% purity) as a yellow solid.

¹H NMR: 400 MHz DMSO-d6 δ 8.43 (s, 1H), 8.33-8.31 (m, 2H), 7.83-7.82 (m, 1H), 7.73-7.72 (m, 1H), 7.59-7.57 (m, 1H), 7.55-7.46 (m, 1H), 7.27 (s, 1H), 7.12-7.11 (m, 1H), 7.11-7.10 (m, 2H), 7.10-7.03 (m, 1H), 6.54-6.52 (m, 1H), 6.24-6.21 (m, 2H), 5.59-5.53 (m, 2H), 4.46-4.23 (m, 2H), 4.19-4.16 (m, 2H), 3.96 (s, 3H), 3.58-3.55 (m, 2H), 3.48-3.45 (m, 2H), 2.45-2.40 (m, 2H), 0.84-0.79 (m, 2H), −0.07-−0.13 (m, 9H).

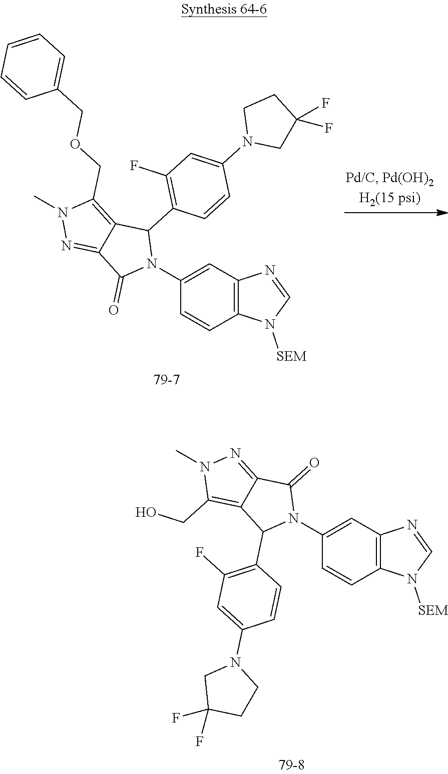 [SMILES: Cn1nc2c(c1COCc1ccccc1)C(c1ccc(N3CCC(F)(F)C3)cc1F)N(c1ccc3c(c1)ncn3COCC[Si](C)(C)C)C2=O ; Cn1nc2c(c1CO)C(c1ccc(N3CCC(F)(F)C3)cc1F)N(c1ccc3c(c1)ncn3COCC[Si](C)(C)C)C2=O]

To a solution of compound 79-7 (950 mg, 1.27 mmol, 1.00 eq) in MeOH (20 mL) was added Pd/C (10%, 0.20 g) and Pd(OH)₂ under N₂ atmosphere. The suspension was degassed and purged with H₂ for 3 times. The mixture was stirred under H₂ (15 Psi) at 50° C. for 12 hrs and at 80° C. for 12 hrs. The reaction mixture was filtered through a pad of celite and concentrated under reduced pressure to give a residue. The residue was purified by column chromatography (SiO₂, DCM:MeOH=1:0 to 10:1, product R_{f}=0.5) to give compound 79-8 (500 mg, 816.04 μmol, 64.0% yield) as a colorless oil.

¹H NMR: 400 MHz DMSO-d6 δ 8.33-8.29 (m, 1H), 7.81-7.68 (m, 1H), 7.56-7.54 (m, 1H), 7.39-7.37 (m, 1H), 7.02-6.98 (m, 1H), 6.49-6.45 (m, 1H), 6.25-6.22 (m, 2H), 5.57-5.56 (m, 2H), 5.33-5.28 (m, 1H), 4.44-4.40 (m, 1H), 4.31-4.27 (m, 1H), 3.94 (s, 3H), 3.61-3.54 (m, 2H), 3.48-3.44 (m, 2H), 2.45-2.40 (m, 2H), 0.84-0.79 (m, 2H), −0.07-−0.12 (m, 9H).

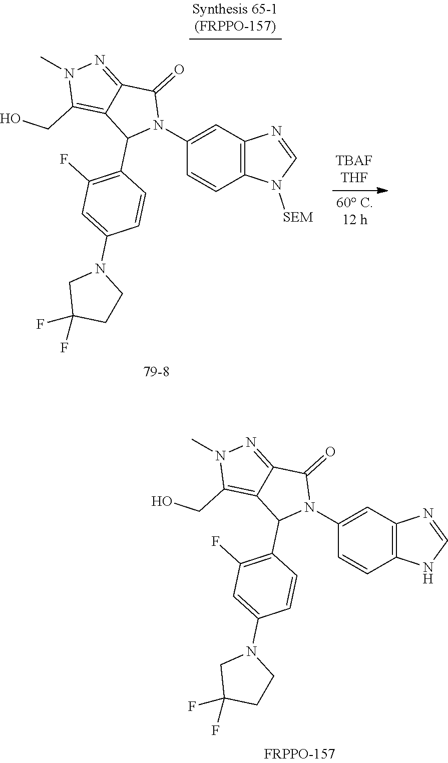 [SMILES: Cn1nc2c(c1CO)C(c1ccc(N3CCC(F)(F)C3)cc1F)N(c1ccc3c(c1)ncn3COCC[Si](C)(C)C)C2=O ; Cn1nc2c(c1CO)C(c1ccc(N3CCC(F)(F)C3)cc1F)N(c1ccc3[nH]cnc3c1)C2=O]

To a solution of compound 79-8 (Scheme 55 and Synthesis 64-6) (50.0 mg, 81.6 μmol, 1.00 eq) in THF (2 mL) was added TBAF (1 M, 0.5 mL, 6.13 eq). The mixture was stirred at 60° C. for 12 hrs. The mixture was concentrated to give a residue. The crude product was purified by reversed-phase HPLC (0.1% NH₃.H₂O in acetonitrile) to give FRPPO-157 (11.41 mg, 23.6 μmol, 28.9% yield, 99.7% purity) as a white solid.

LCMS: RT=0.858 min, m/z=483.5 (M+1)⁺.

HPLC: RT=2.214 min.

¹H NMR: 400 MHz MeOD δ 8.15 (s, 1H), 7.60-7.49 (m, 2H), 7.30 (s, 1H), 6.97-6.93 (m, 1H), 6.44 (s, 1H), 6.29-6.27 (m, 1H), 6.22-6.18 (m, 1H), 4.58-4.55 (m, 2H), 4.48-4.44 (m, 1H), 4.05 (s, 3H), 3.59-3.52 (m, 2H), 3.43-3.39 (m, 2H), 2.47-2.38 (m, 2H).

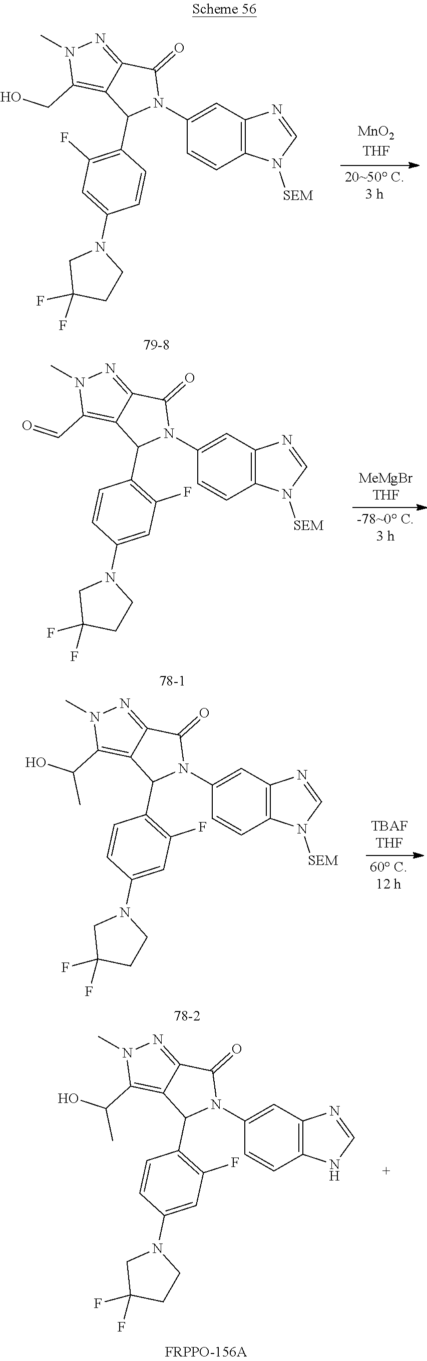

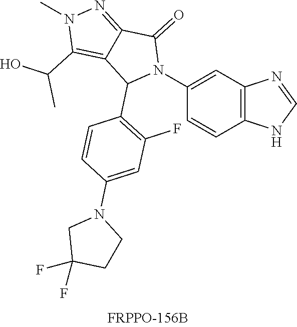 [SMILES: CC(O)c1c2c(nn1C)C(=O)N(c1ccc3[nH]cnc3c1)C2c1ccc(N2CCC(F)(F)C2)cc1F]

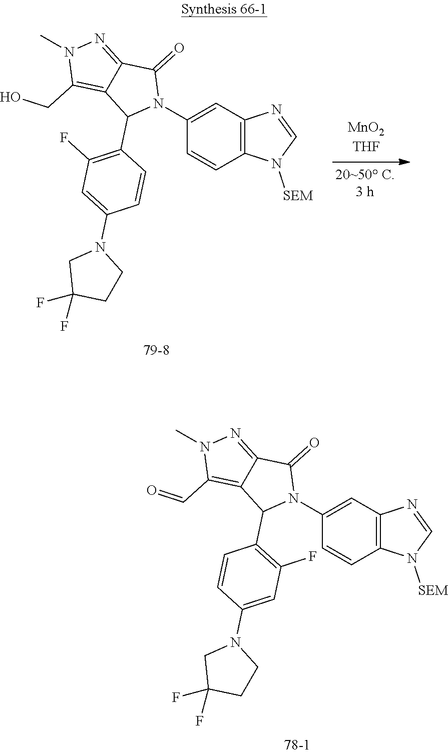 [SMILES: Cn1nc2c(c1CO)C(c1ccc(N3CCC(F)(F)C3)cc1F)N(c1ccc3c(c1)ncn3COCC[Si](C)(C)C)C2=O ; Cn1nc2c(c1C=O)C(c1ccc(N3CCC(F)(F)C3)cc1F)N(c1ccc3c(c1)ncn3COCC[Si](C)(C)C)C2=O]

To a solution of compound 79-8 (100 mg, 163 μmol, 1.00 eq) in THF (3 mL) was added MnO₂ (300 mg, 3.45 mmol, 21.1 eq), the mixture was stirred at 20° C. for 16 hrs. The mixture was filtered and concentrated to give a residue to give compound 78-1 (30 mg, crude) was obtained as a yellow oil, which was used in the next step directly.

LCMS: RT=0.933 min, m/z=611.4 (M+1)⁺.

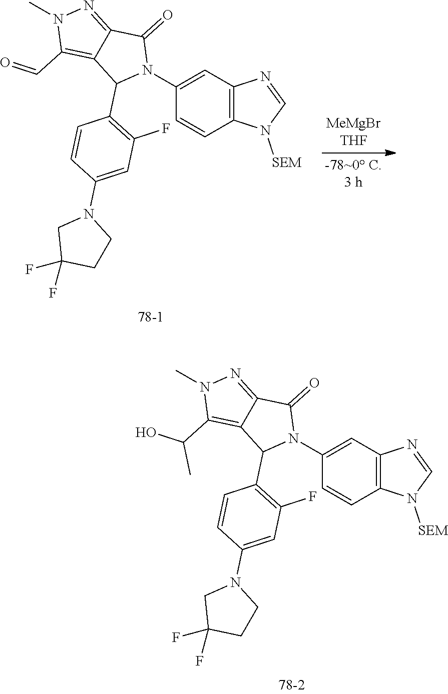 [SMILES: Cn1nc2c(c1C=O)C(c1ccc(N3CCC(F)(F)C3)cc1F)N(c1ccc3c(c1)ncn3COCC[Si](C)(C)C)C2=O ; CC(O)c1c2c(nn1C)C(=O)N(c1ccc3c(c1)ncn3COCC[Si](C)(C)C)C2c1ccc(N2CCC(F)(F)C2)cc1F]

To a solution of compound 78-1 (30 mg, 49.1 μmol, 1.00 eq) in THF (3 mL) was added MeMgBr (3 M, 33.5 μL, 2.05 eq) at −78° C. Then mixture was stirred at 0° C. for 3 hrs. The mixture was quenched by NH₄Cl (3 mL) at 0° C., and then diluted with water (10 mL) and extracted with Ethyl acetate (5 mL*2). The combined organic layers were washed with brine (5 mL*2), dried over Na₂SO₄, filtered and concentrated under reduced pressure to give a residue to give compound 78-2 (31 mg, crude) was obtained as a yellow oil and used in the next step directly.

LCMS: RT=0.917 min. m/z=627.4 (M+1)⁺

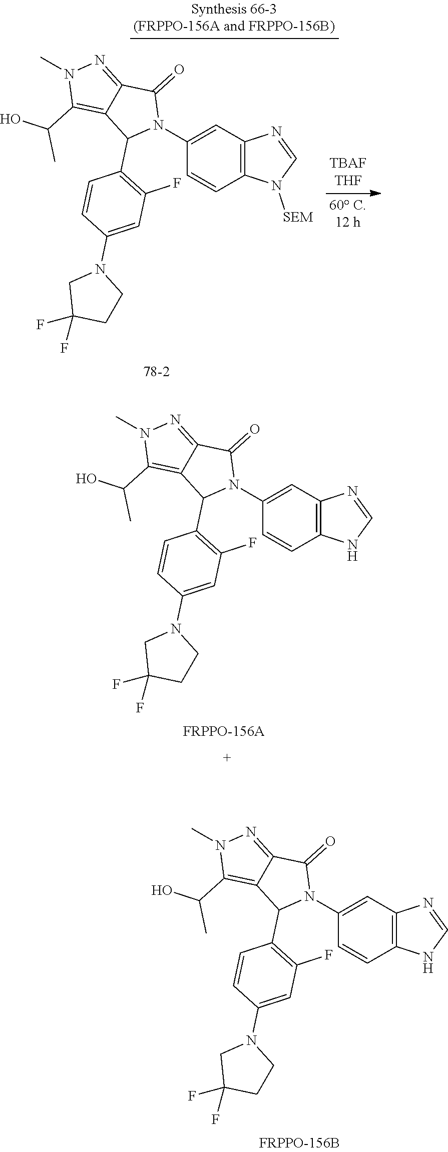 [SMILES: CC(O)c1c2c(nn1C)C(=O)N(c1ccc3c(c1)ncn3COCC[Si](C)(C)C)C2c1ccc(N2CCC(F)(F)C2)cc1F ; CC(O)c1c2c(nn1C)C(=O)N(c1ccc3[nH]cnc3c1)C2c1ccc(N2CCC(F)(F)C2)cc1F ; CC(O)c1c2c(nn1C)C(=O)N(c1ccc3[nH]cnc3c1)C2c1ccc(N2CCC(F)(F)C2)cc1F]

To a solution of compound 78-2 (30 mg, 47.8 μmol, 1.00 eq) in THF (1 mL) was added TBAF (1 M, 0.3 mL, 6.27 eq), the mixture was heated to 60° C. and held for 12 hrs. The mixture was concentrated to give a residue to give the crude product which was used to purified by prep-HPLC (basic condition column: Waters Xbridge 150*25 mm*5 μm; mobile phase: 0.05% ammonia hydroxide, A-water, B-ACN; gradient: 16%-46% B %, 9 min) to give:

FRPPO-156A (1.53 mg, 3.08 μmol, 6.4% yield) as an off-white solid.

LCMS: RT=0.772 min, m/z=497.2 (M+1)⁺.

HPLC: RT=1.625 min.

¹H NMR: 400 MHz MeOD δ 8.15 (s, 1H), 7.56-7.52 (m, 2H), 7.26-7.23 (m, 1H), 6.92 (s, 1H), 6.41 (s, 1H), 6.29-6.27 (m, 1H), 6.18-6.14 (m, 1H), 4.98-4.93 (m, 1H), 4.09 (s, 3H), 3.58-3.52 (m, 2H), 3.42-3.31 (m, 2H), 2.48-2.37 (m, 2H), 1.36-1.34 (m, 3H).

FRPPO-156B (1.17 mg, 2.36 μmol, 4.9% yield) as an off-white solid.

LCMS: RT=0.778 min, m/z=497.2 (M+1)⁺.

HPLC: RT=1.665 min.

¹H NMR: 400 MHz MeOD δ 8.31 (s, 1H), 7.62 (s, 1H), 7.58-7.56 (m, 2H), 7.34-7.31 (m, 1H), 6.91 (s, 1H), 6.47 (s, 1H), 6.29-6.26 (m, 1H), 6.18-6.14 (m, 1H), 4.95-4.93 (m, 1H), 4.05 (s, 3H), 3.58-3.51 (m, 2H), 3.41-3.31 (m, 2H), 2.48-2.37 (m, 2H), 1.15-1.13 (m, 3H).

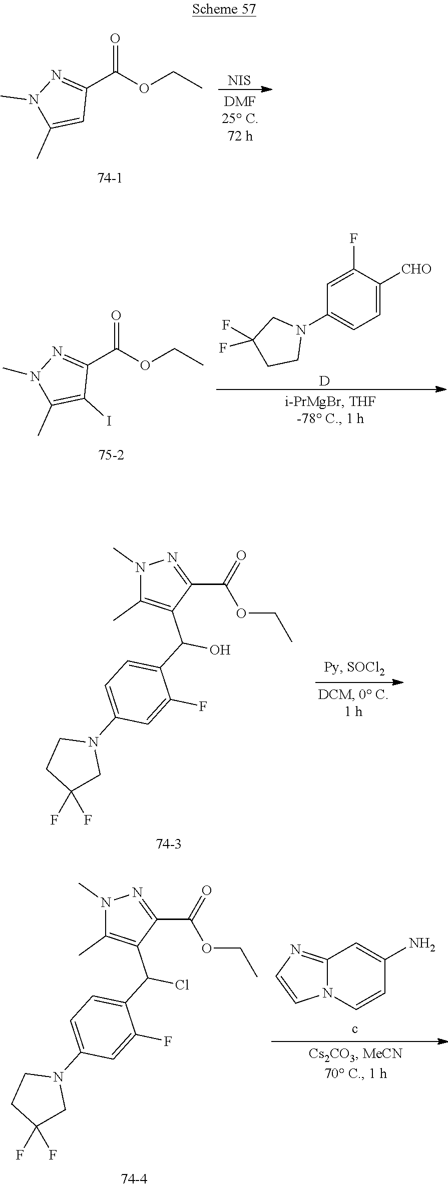

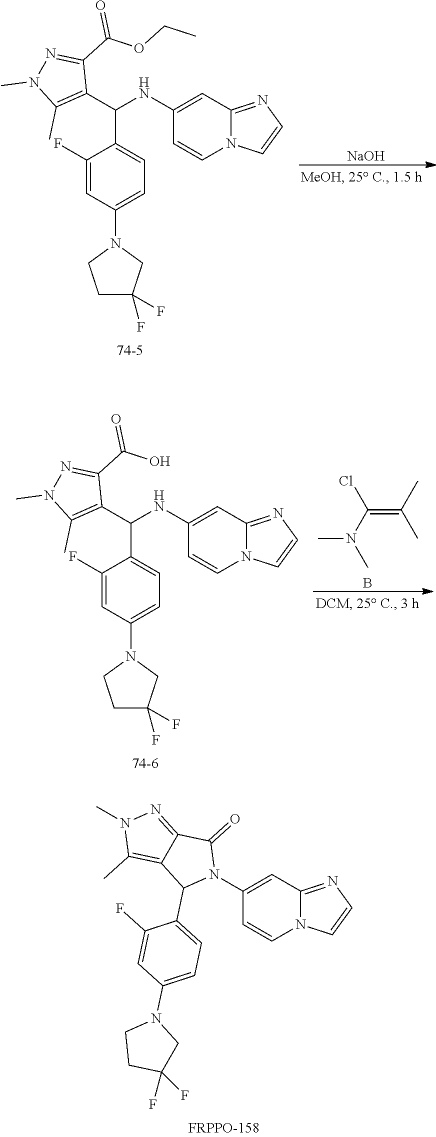

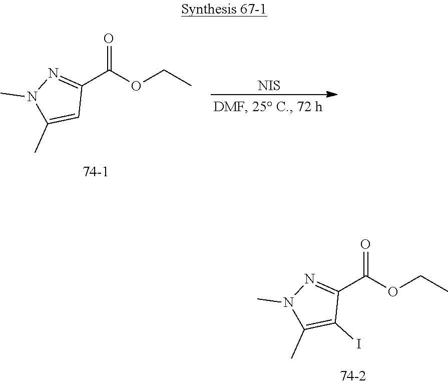

To a solution of ethyl compound 74-1 (20.0 g, 119 mmol, 1.00 eq) in DMF (100 mL) was added NIS (29.4 g, 131 mmol, 1.10 eq). The mixture was stirred at 25° C. for 72 hrs. TLC (petroleum ether:ethyl acetate=1:1) showed compound 74-1 (R_{f}=0.3) was remained and new spots (R_{f}=0.7, 0.4) formed. The mixture was poured into water (1 L) and extracted with EtOAc (500 mL*3). The combined organic layers were dried over anhydrous sodium sulfate, filtrated and concentrated to give a residue. The residue was purified by column chromatography (silica gel, petroleum ether:ethyl acetate=20:1 to 5:1) to get compound 74-2 (petroleum ether:ethyl acetate=1:1, R_{f}=0.4) (22.0 g, 74.8 mmol, 62.9% yield) as a yellow solid.

¹H NMR: 400 MHz CDCl₃4.45-4.36 (m, 2H), 3.95 (s, 3H), 2.37 (s, 3H), 1.44-1.41 (m, 3H).

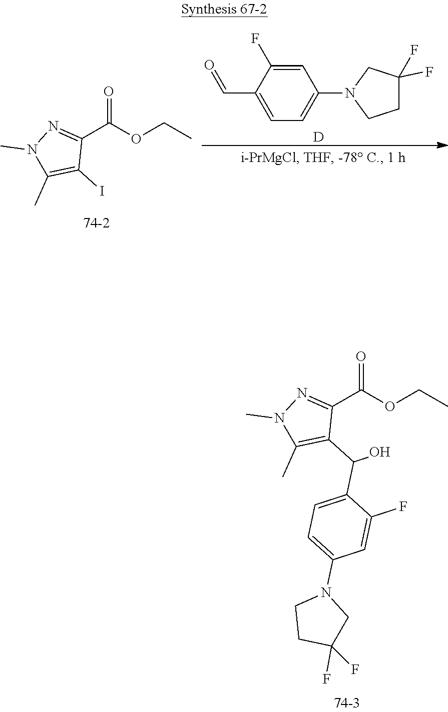

To a solution of compound 74-2 (3.00 g, 10.2 mmol, 1.00 eq) in THF (30 mL) was added i-PrMgCl (2.0 M, 7.65 mL, 1.50 eq) at −78° C. The mixture was stirred at −78° C. for 0.5 hr and then a solution of compound D (Scheme 40 and Synthesis 44-1) (2.85 g, 12.2 mmol, 1.20 eq) in THF (20 mL) was added to the mixture and the mixture was stirred at −78° C. for 0.5 hr. The mixture was poured into water (200 mL) and extracted with EtOAc (80 mL*2). The combined organic layers were dried over anhydrous sodium sulfate, filtrated and concentrated to give a residue. The residue was purified by column chromatography (silica gel, petroleum ether:ethyl acetate=10:1 to 1:1) to get compound 74-3 (petroleum ether:ethyl acetate=1:1, R_{f}=0.3) (2.08 g, 5.23 mmol, 51.3% yield) as a white solid.

¹H NMR: 400 MHz CDCl₃ 7.36-7.32 (m, 1H), 6.27-6.26 (m, 1H), 6.25-6.24 (m, 1H), 6.18-5.99 (m, 1H), 5.69-5.67 (m, 1H), 4.45-4.39 (m, 2H), 3.85 (s, 3H), 3.66-3.58 (m, 2H), 3.48-3.45 (m, 2H), 3.52-3.41 (m, 2H), 2.28 (s, 3H), 1.41-1.38 (m, 3H).

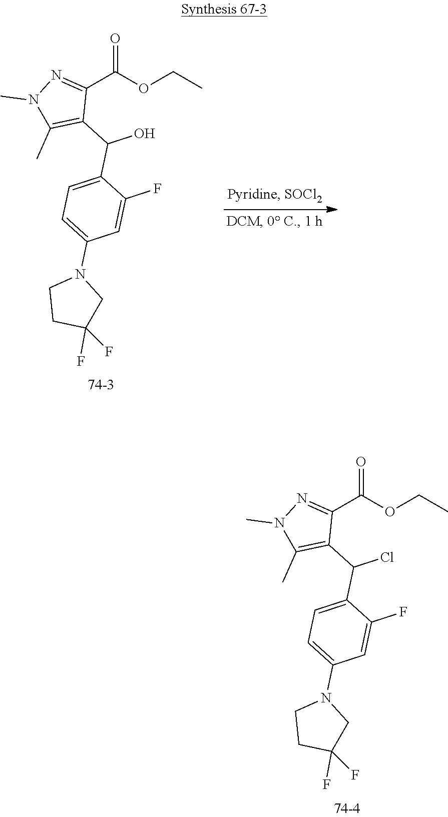 [SMILES: CCOC(=O)c1nn(C)c(C)c1C(O)c1ccc(N2CCC(F)(F)C2)cc1F ; CCOC(=O)c1nn(C)c(C)c1C(Cl)c1ccc(N2CCC(F)(F)C2)cc1F]

To a solution of compound 74-3 (500 mg, 1.26 mmol, 1.00 eq) in DCM (5 mL) was added Pyridine (196 mg, 2.48 mmol, 0.2 mL, 1.97 eq) and SOCl₂ (328 mg, 2.76 mmol, 0.2 mL, 2.19 eq) at 0° C. The mixture was stirred at 0° C. for 1 hr. TLC (petroleum ether:ethyl acetate=1:1) showed compound 74-3 (R_{f}=0.5) was consumed and new spot (R_{f}=0.7) formed. The mixture was concentrated to give a residue. The crude product 74-4 was used in the next step without purification.

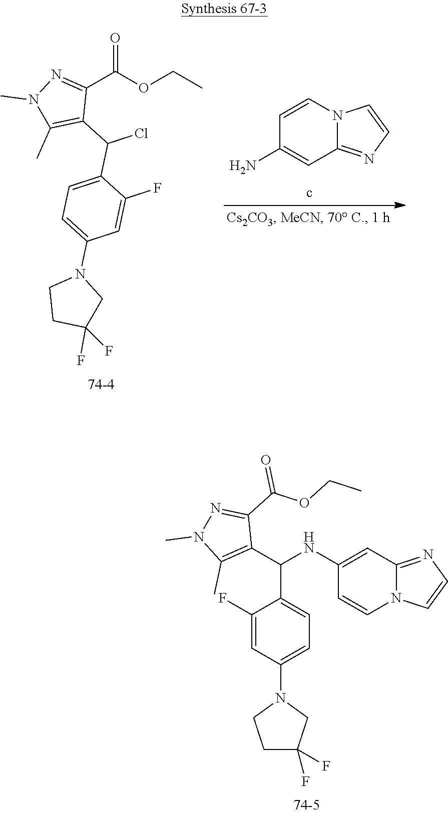 [SMILES: CCOC(=O)c1nn(C)c(C)c1C(Cl)c1ccc(N2CCC(F)(F)C2)cc1F ; Nc1ccn2ccnc2c1 ; CCOC(=O)c1nn(C)c(C)c1C(Nc1ccn2ccnc2c1)c1ccc(N2CCC(F)(F)C2)cc1F]

To a solution of compound 74-4 (500 mg, 1.20 mmol, 1.00 eq) and compound C (160 mg, 1.20 mmol, 1.00 eq) in CH₃CN (10 mL) was added Cs₂CO₃ (785 mg, 2.41 mmol, 2.00 eq). The mixture was stirred at 70° C. for 1 hr. The mixture was filtrated and the filtrate was concentrated under reduced pressure to give a residue. The residue was purified by prep-TLC (SiO₂, DCM:MeOH=10:1) to get compound 74-5 (DCM:MeOH=10:1, R_{f}=0.25) (450 mg, crude) as a yellow solid.

¹H NMR: 400 MHz CDCl₃ δ 8.01-7.88 (m, 1H), 7.31-7.25 (m, 1H), 7.23-7.11 (m, 2H), 6.83-6.27 (m, 2H), 6.24-5.97 (m, 3H), 4.43-438 (m, 2H), 3.85-3.62 (m, 3H), 3.59-3.48 (m, 2H), 3.47-3.43 (m, 2H), 2.51-2.26 (m, 5H), 1.41-1.33 (m, 3H).

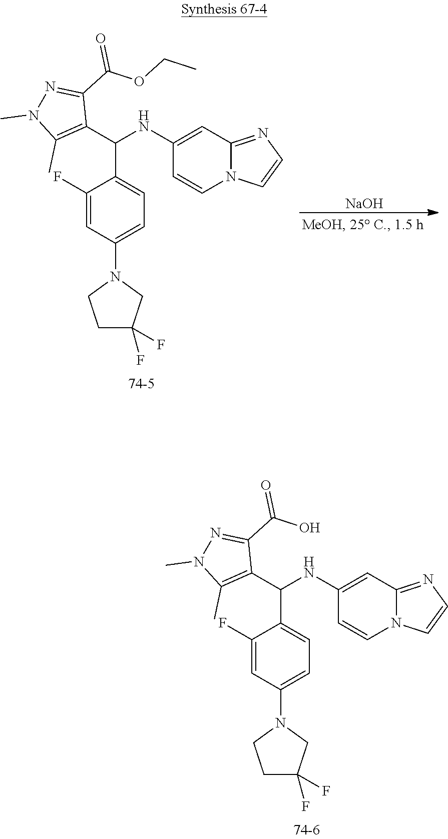 [SMILES: CCOC(=O)c1nn(C)c(C)c1C(Nc1ccn2ccnc2c1)c1ccc(N2CCC(F)(F)C2)cc1F ; "NaOH" ; Cc1c(C(Nc2ccn3ccnc3c2)c2ccc(N3CCC(F)(F)C3)cc2F)c(C(=O)O)nn1C]

To a solution of compound 74-5 (100 mg, 195 μmol, 1.00 eq) in MeOH (5 mL) was added NaOH (2 M, 250 μL, 2.56 eq). The mixture was stirred at 25° C. for 1.5 hrs. The mixture was concentrated to give a residue. The crude product was purified by reversed-phase HPLC (0.1% HCl condition) to get compound 74-6 (50.0 mg, 68.9 μmol, 35.3% yield, 66.8% purity) as a yellow solid.

LCMS: RT=0.694 min, m/z=485.3 (M+1)⁺.

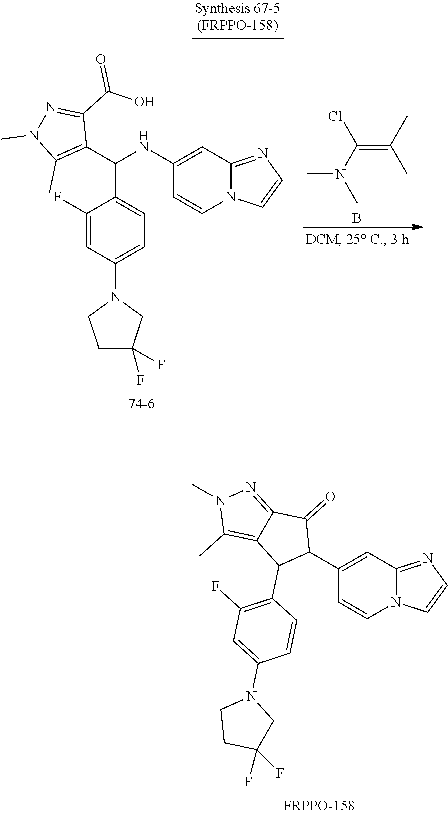

To a solution of compound 74-6 (50.0 mg, 68.9 μmol, 1.00 eq) in DCM (5 mL) was added compound B (30.0 mg, 225 μmol, 29.7 μL, 3.26 eq). The mixture was stirred at 25° C. for 3 hrs. The mixture was concentrated to give a residue. The residue was purified by prep-HPLC (basic condition, column: Waters Xbridge 150*25 mm*5 μm; mobile phase: [water (0.05% ammonia hydroxide v/v)-ACN]; B %: 27%-57%, 10 min) to get compound FRPPO-158 (11.43 mg, 23.8 μmol, 34.5% yield, 97.1% purity) as a off-white solid.

LCMS: RT=0.941 min, m/z=467.3 (M+1)⁺.

HPLC: RT=2.830 min.

¹H NMR: 400 MHz MeOD δ 8.32 (d, J=7.6 HZ, 1H), 7.72-7.57 (m, 1H), 7.56-7.48 (m, 1H), 7.40-7.39 (m, 1H), 7.39-7.38 (m, 1H), 7.00-6.96 (m, 1H), 6.49 (s, 1H), 6.32-6.28 (m, 2H), 3.93 (s, 3H), 3.61-3.54 (m, 2H), 3.44-3.31 (m, 2H), 2.50-2.39 (m, 2H), 2.19 (s, 3H).

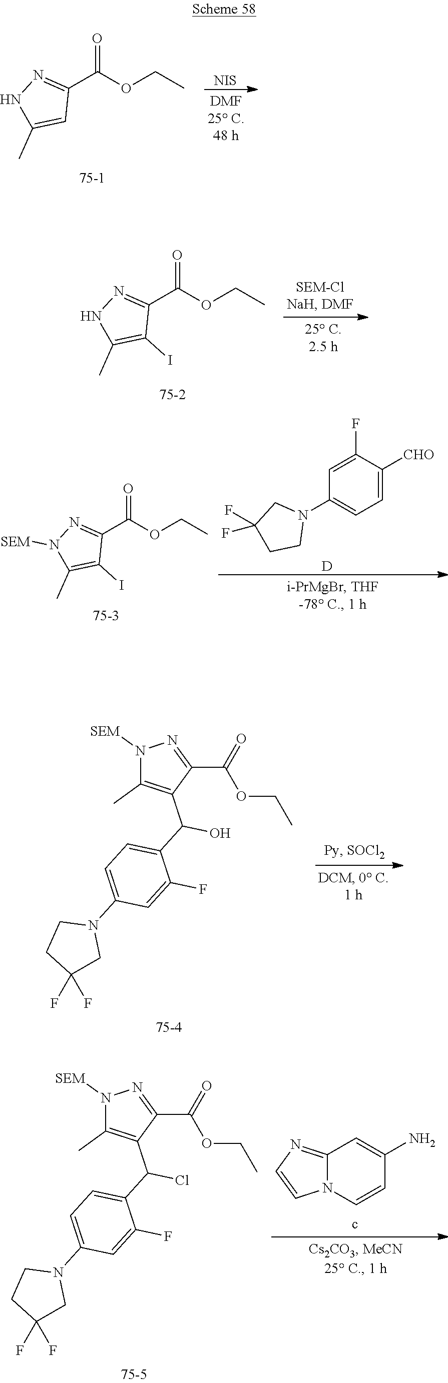

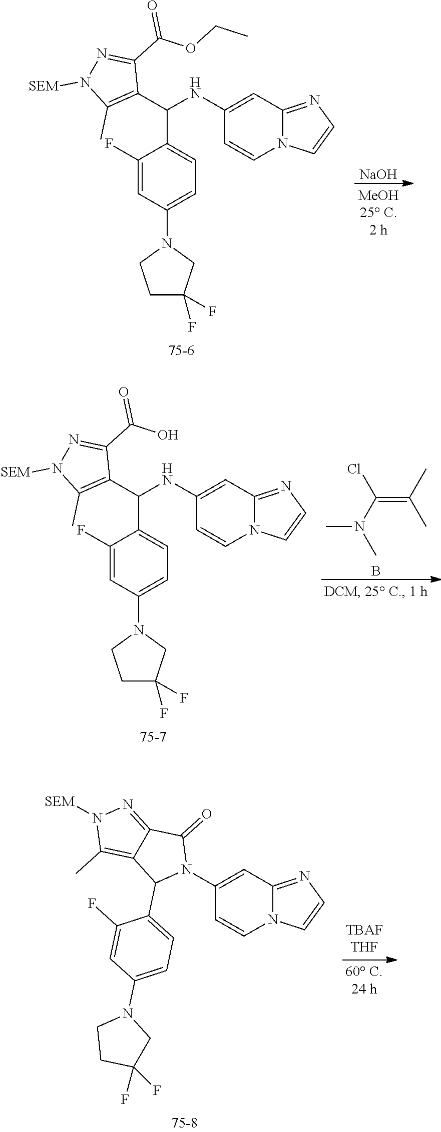

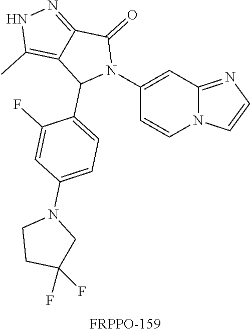 [SMILES: Cc1[nH]nc2c1C(c1ccc(N3CCC(F)(F)C3)cc1F)N(c1ccn3ccnc3c1)C2=O]

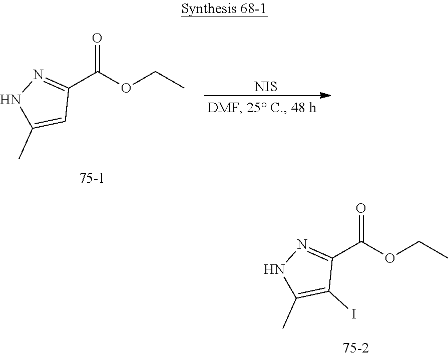

To a solution of compound 75-1 (20.0 g, 130 mmol, 1.00 eq) in DMF (100 mL) was added NIS (37.4 g, 169 mmol, 1.30 eq). The mixture was stirred at 25° C. for 48 hrs. The mixture was poured into water (1 L) and filtrated. The solid was collected and concentrated to give compound 75-2 (32.0 g, 114 mmol, 88.1% yield) as a yellow solid.

¹H NMR: 400 MHz DMSO-d6 δ 13.61 (s, 1H), 4.28-4.23 (m, 2H), 2.24 (s, 3H), 1.31-1.27 (m, 3H).

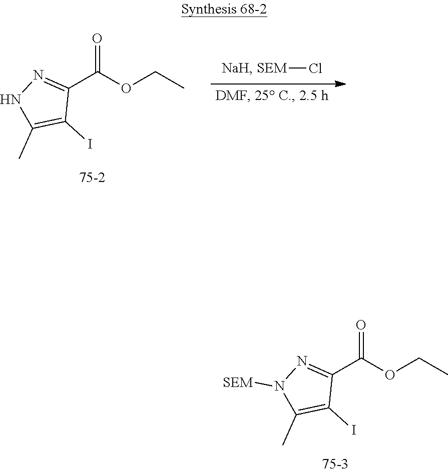

To a solution of compound 75-2 (5.00 g, 17.9 mmol, 1.00 eq) in DMF (50 mL) was added NaH (860 mg, 21.5 mmol, 60% purity, 1.20 eq). The mixture was stirred at 25° C. for 0.5 hr, then SEM-Cl (2.98 g, 17.9 mmol, 3.16 mL, 1.00 eq) was added and the mixture was stirred at 25° C. for 2 hrs. The mixture was poured into water (200 mL) and extracted with EtOAc (100 mL*2). The combined organic layers were dried over anhydrous sodium sulfate, filtrated and concentrated to give a residue. The residue was purified by column chromatography (silica gel, petroleum ether:ethyl acetate=10:1) to give compound 75-3 (R_{f}=0.7, 0.8, petroleum ether:ethyl acetate=1:1) (6.80 g, 16.6 mmol, 92.8% yield) as a yellow oil.

¹H NMR: 400 MHz CDCl₃ δ 5.78-5.53 (m, 2H), 4.46-4.39 (m, 2H), 3.57-3.52 (m, 2H), 2.43-2.30 (m, 3H), 1.45-1.41 (m, 3H), 0.87-0.86 (m, 2H), −0.02-−0.05 (m, 9H).

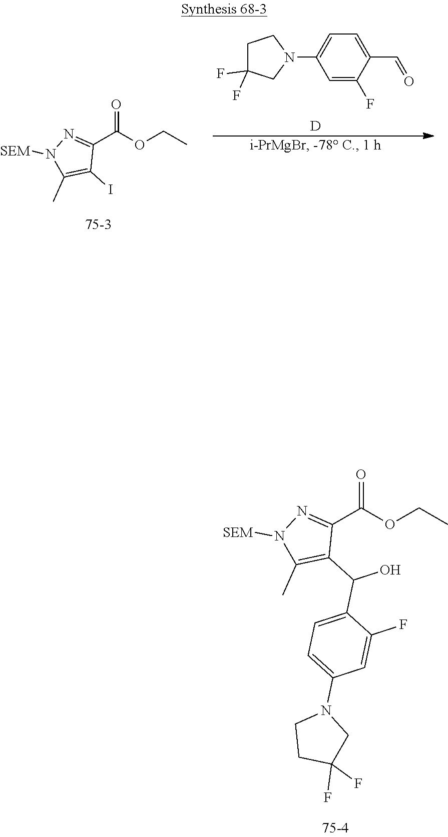

To a solution of compound 75-3 (5.00 g, 12.2 mmol, 1.00 eq) in THF (50 mL) was added i-PrMgBr (2.00 M, 9 mL, 1.48 eq) at −78° C. The mixture was stirred at −78° C. for 0.5 hr and then a solution of compound D (Scheme 40 and Synthesis 44-1) (3.40 g, 14.6 mmol, 1.20 eq) in THF (30 mL) was added to the mixture and the mixture was stirred at −78° C. for 0.5 hr. The mixture was poured into water (200 mL) and extracted with EtOAc (100 mL*2). The combined organic layers were dried over anhydrous sodium sulfate, filtrated and concentrated to give a residue. The residue was purified by column chromatography (silica gel, petroleum ether:ethyl acetate=10:1 to 5:1) to get compound 75-4 (petroleum ether:ethyl acetate=2:1, R_{f}=0.50 & 0.45) (4.00 g, 7.79 mmol, 63.9% yield) as a yellow oil.

¹H NMR: 400 MHz CDCl₃ δ 7.26-7.21 (m, 1H), 6.27-6.19 (m, 3H), 5.75-5.62 (m, 2H), 4.43-4.38 (m, 2H), 3.67-3.60 (m, 2H), 3.51-3.47 (m, 2H), 2.52-2.45 (m, 2H), 2.34-2.22 (m, 3H), 1.39-1.35 (m, 3H), 1.29-1.25 (m, 2H), 0.89-0.85 (m, 2H), −0.02-−0.04 (m, 9H).

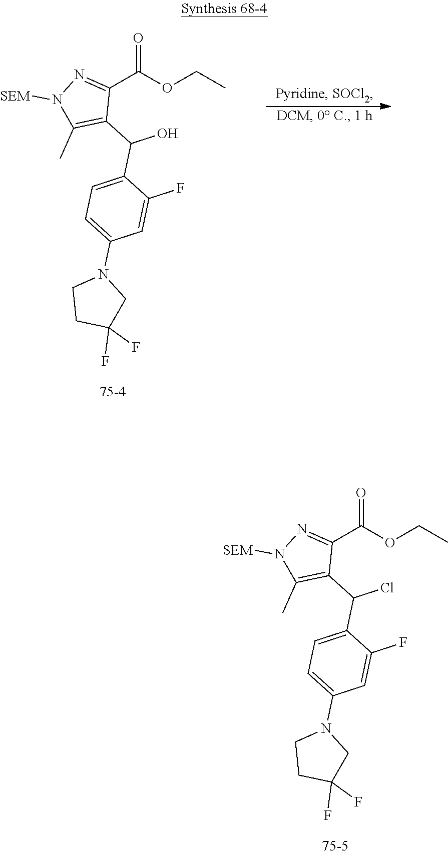 [SMILES: CCOC(=O)c1nn(COCC[Si](C)(C)C)c(C)c1C(O)c1ccc(N2CCC(F)(F)C2)cc1F ; "Pyridine, SOCl2," ; "DCM, 0° C., 1 h" ; CCOC(=O)c1nn(COCC[Si](C)(C)C)c(C)c1C(Cl)c1ccc(N2CCC(F)(F)C2)cc1F]

To a solution of compound 75-4 (1.00 g, 1.95 mmol, 1.00 eq) in DCM (10 mL) was added Pyridine (294 mg, 3.72 mmol, 0.30 mL, 1.91 eq) and SOCl₂ (492 mg, 4.14 mmol, 0.30 mL, 2.12 eq) at 0° C. The mixture was stirred at 0° C. for 1 hr. TLC (petroleum ether:ethyl acetate=3:1) showed the compound 75-4 (R_{f}=0.3) was consumed and new spot (R_{f}=0.6) formed. The mixture was concentrated to give compound 75-5 (1.00 g, crude) as a yellow oil.

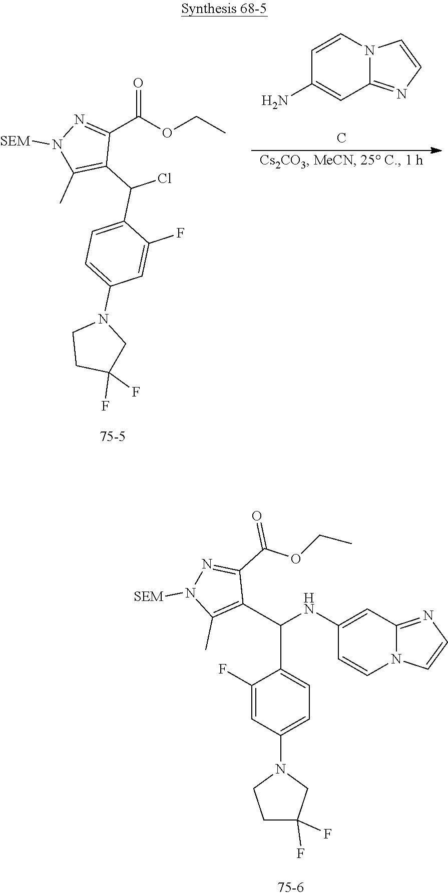

To a solution of compound 75-5 (1.00 g, 1.88 mmol, 1.00 eq) and compound C (330 mg, 2.48 mmol, 1.32 eq) in CH₃CN (10 mL) was added Cs₂CO₃ (1.22 g, 3.76 mmol, 2.00 eq). The mixture was stirred at 25° C. for 1 hr. The mixture was filtrated and the filtrate was concentrated to give a residue. The residue was purified by column chromatography (silica gel, Dichloromethane:Methanol=50:1 to 10:1) to get compound 75-6 (550 mg, crude) as a yellow solid.

¹H NMR: 400 MHz CDCl₃ δ 7.85-7.74 (m, 1H), 7.30-7.27 (m, 1H), 7.17-7.13 (m, 1H), 6.53-6.36 (m, 1H), 6.28-6.01 (m, 3H), 5.81-5.45 (m, 2H), 4.42-4.32 (m, 2H), 3.65-3.46 (m, 7H), 2.52-2.15 (m, 5H), 1.40-1.26 (m, 4H), 0.89-0.84 (m, 2H), −0.05-−0.08 (m, 9H).

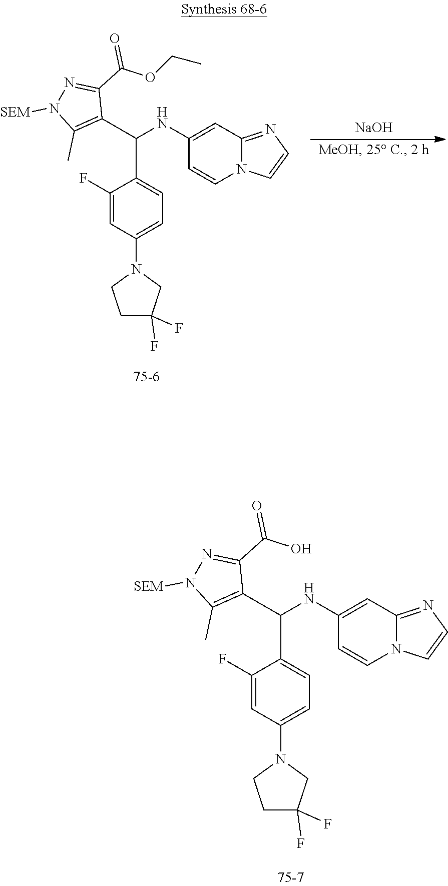 [SMILES: CCOC(=O)c1nn(COCC[Si](C)(C)C)c(C)c1C(Nc1ccn2ccnc2c1)c1ccc(N2CCC(F)(F)C2)cc1F ; "NaOH" ; "MeOH, 25° C., 2 h" ; Cc1c(C(Nc2ccn3ccnc3c2)c2ccc(N3CCC(F)(F)C3)cc2F)c(C(=O)O)nn1COCC[Si](C)(C)C]

To a solution of compound 75-6 (450 mg, 716 μmol, 1.00 eq) in MeOH (10 mL) was added NaOH (2 M, 1.13 mL, 3.14 eq). The mixture was stirred at 25° C. for 2 hrs. The mixture was concentrated. The residue was diluted with H₂O (10 mL) and adjusted to pH=5 with HCl solution (1 M). The mixture was extracted with EtOAc (10 mL*2). The organic layers were dried over anhydrous sodium sulfate, filtrated and concentrated to give compound 75-7 (400 mg, crude) as a yellow solid.

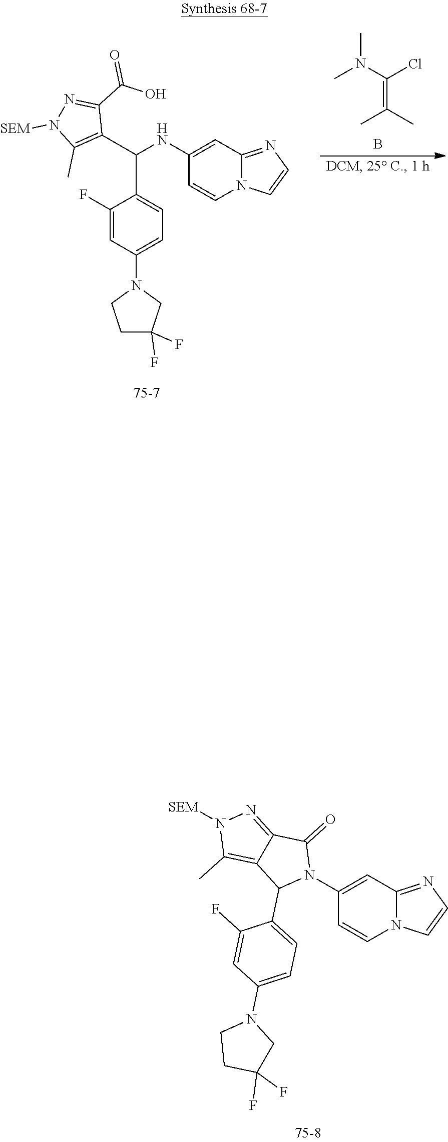

A mixture of compound 75-7 (400 mg, 666 μmol, 1.00 eq) and compound B (215 mg, 1.61 mmol, 213 μL, 2.42 eq) in DCM (5 mL) was stirred at 25° C. for 1 hr. The mixture was diluted with DCM (30 mL) and washed with water (30 mL). The organic layer was concentrated to give a residue. The crude product was purified by reversed-phase HPLC (0.1% FA condition) to give compound 75-8 (100 mg, 123 μmol, 18.5% yield, 71.6% purity) as a yellow solid.

LCMS: RT=0.918 min, m/z=583.3 (M+1)⁺.

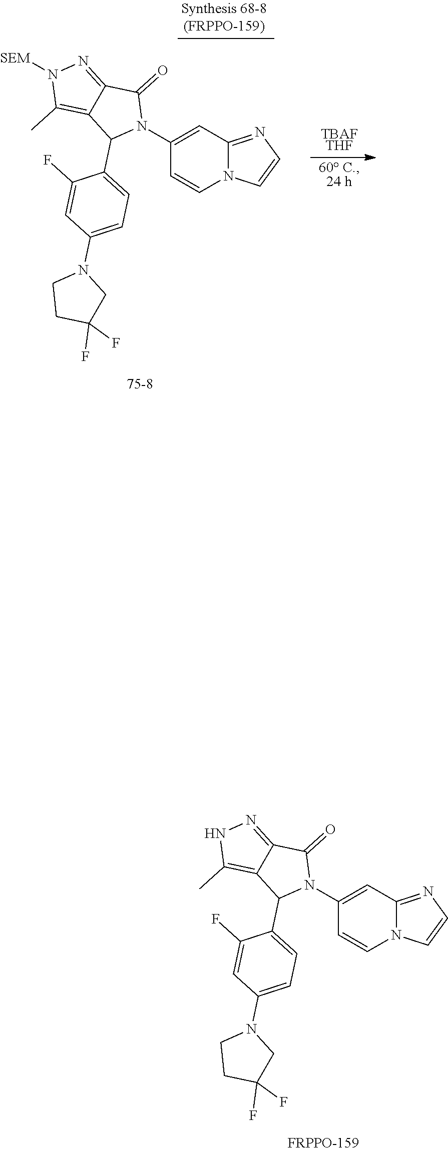 [SMILES: Cc1c2c(nn1COCC[Si](C)(C)C)C(=O)N(c1ccn3ccnc3c1)C2c1ccc(N2CCC(F)(F)C2)cc1F ; "60° C.,24 h" ; "TBAFTHF" ; Cc1[nH]nc2c1C(c1ccc(N3CCC(F)(F)C3)cc1F)N(c1ccn3ccnc3c1)C2=O]

To a solution of compound 75-8 (80.0 mg, 98.3 μmol, 1.00 eq) in THF (5 mL) was added TBAF (1 M, 197 μL, 2.00 eq). The mixture was stirred at 60° C. for 24 hrs. The mixture was concentrated to give a residue. The residue was purified by prep-TLC (SiO₂, DCM MeOH=10:1) to give a residue (DCM:MeOH=10:1, R_{f}=0.4 &0.5). The crude product was purified by reversed-phase HPLC (0.1% NH₃.H₂O) to give compound FRPPO-159 (7.43 mg, 15.3 μmol, 15.6% yield, 93.1% purity) as a off-white solid.

LCMS: RT=0.897 min, m/z=453.2 (M+1)⁺.

HPLC: RT=2.264 min.

¹H NMR: 400 MHz MeOD δ 8.34-8.33 (m, 1H), 7.73 (s, 1H), 7.58 (s, 1H), 7.48 (s, 1H), 7.40-7.38 (m, 1H), 7.00-6.95 (m, 1H), 6.54-6.51 (m, 1H), 6.32-6.29 (m, 2H), 3.61-3.54 (m, 2H), 3.45-3.31 (m, 2H), 2.48-2.41 (m, 2H), 2.19 (m, 3H).

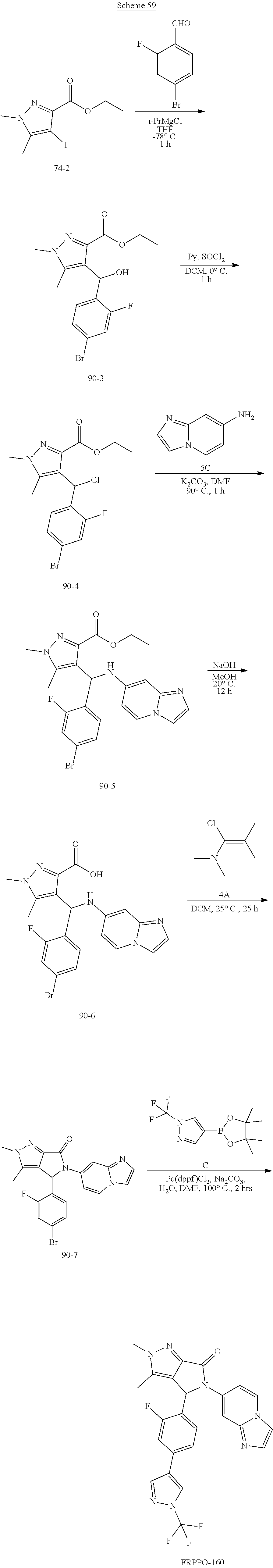

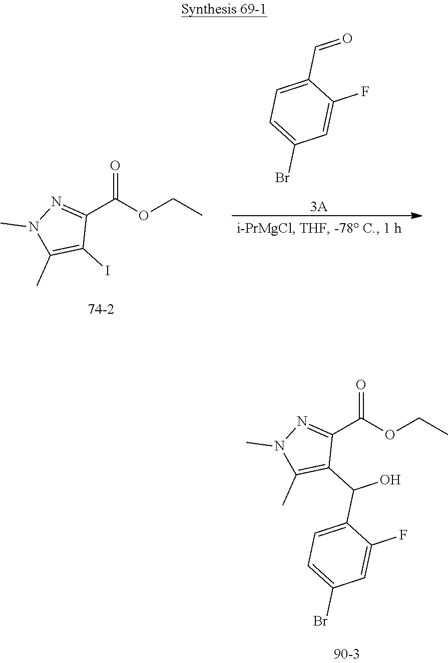

To a solution of compound 74-2 (Scheme 57 and Synthesis 67-1) (4.80 g, 16.3 mmol, 1.00 eq) in THF (40 mL) was added i-PrMgCl (2 M, 12.2 mL, 1.50 eq), the mixture was stirred at −78° C. for 0.5 hr, then compound 3A (3.98 g, 19.6 mmol, 1.20 eq) in THF (12 mL) was added, the mixture was stirred at −78° C. for 0.5 hr. The reaction mixture was partitioned between water 200 mL and Ethyl acetate 200 mL. The organic phase was separated, washed with Ethyl acetate 400 mL (200 mL*2), dried over Na₂SO₄, filtered and concentrated under reduced pressure to give a residue. The crude product was purified by reversed-phase HPLC (0.1% FA in acetonitrile) to give compound 90-3 (2.50 g, 6.73 mmol, 41.2% yield) as a colorless oil.

¹H NMR: 400 MHz CDCl₃ δ 7.52-7.48 (m, 1H), 7.27-7.25 (m, 1H), 7.14-7.11 (m, 1H), 5.97 (s, 1H), 5.84 (s, 1H), 4.43-4.38 (m, 2H), 3.86 (s, 3H), 2.29 (s, 3H), 1.41-1.37 (m, 3H).

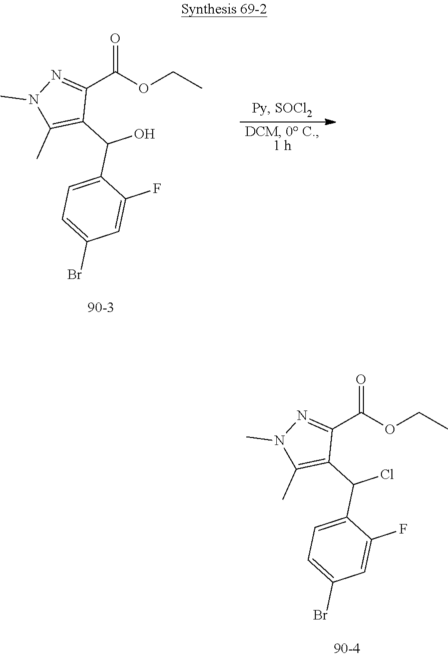 [SMILES: CCOC(=O)c1nn(C)c(C)c1C(O)c1ccc(Br)cc1F ; "DCM, 0° C.,1 h" ; "Py, SOCl2" ; CCOC(=O)c1nn(C)c(C)c1C(Cl)c1ccc(Br)cc1F]

To a solution of compound 90-3 (1.00 g, 2.69 mmol, 1.00 eq) in DCM (20 mL) was added Py (1.00 g, 12.6 mmol, 0.6 mL, 4.69 eq) and SOCl₂ (820 mg, 6.89 mmol, 500 μL, 2.56 eq) at 0° C. The mixture was stirred at 0° C. for 1 hr. TLC (petroleum ether:ethyl acetate=1:1) showed compound 90-3 (R_{f}=0.5) was consumed, and new spot (R_{f}=0.6) formed. The reaction mixture was concentrated under reduced pressure to give compound 90-4 (1.05 g, 2.69 mmol, 100.00% yield) as a yellow solid.

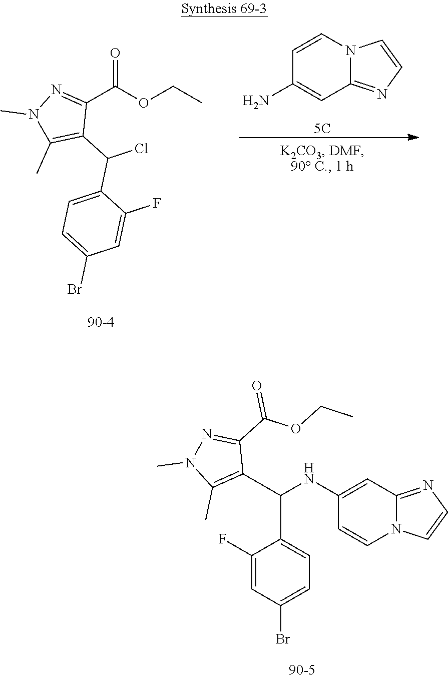

To a solution of compound 90-4 (1.05 g, 2.69 mmol, 1.00 eq) in DMF (10 mL) was added K₂CO₃ (1.11 g, 8.04 mmol, 2.98 eq) and compound 5C (305 mg, 2.29 mmol, 0.85 eq). The mixture was stirred at 90° C. for 1 hr. The reaction mixture was partitioned between water 100 mL and Ethyl acetate 150 mL. The organic phase was separated, washed with brine 150 mL, dried over Na₂SO₄, filtered and concentrated under reduced pressure to give a residue. The crude product was purified by reversed-phase HPLC (0.1% NH₃.H₂O in acetonitrile) to give compound 90-5 (700 mg, 1.44 mmol, 53.4% yield) as a yellow solid.

¹H NMR: 400 MHz CDCl₃ δ 7.84-7.82 (m, 1H), 7.45-7.41 (m, 1H), 7.35 (s, 1H), 7.29-7.27 (m, 1H), 7.18-7.16 (m, 1H), 7.15-7.14 (m, 1H), 6.43-6.42 (m, 1H), 6.37-6.35 (m, 1H), 5.97-5.95 (m, 1H), 4.44-4.37 (m, 2H), 3.85 (s, 3H), 2.40 (s, 3H), 1.41-1.36 (m, 3H).

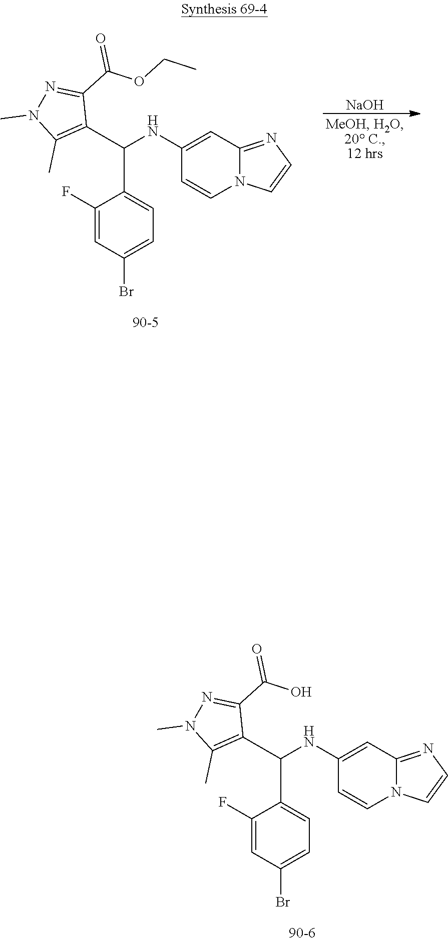 [SMILES: CCOC(=O)c1nn(C)c(C)c1C(Nc1ccn2ccnc2c1)c1ccc(Br)cc1F ; "NaOH" ; "MeOH, H2O,20° C.,12 hrs" ; Cc1c(C(Nc2ccn3ccnc3c2)c2ccc(Br)cc2F)c(C(=O)O)nn1C]

To a solution of compound 90-5 (650 mg, 1.34 mmol, 1.00 eq) in MeOH (4 mL) was added NaOH (650 mg, 16.2 mmol, 12.1 eq) in H₂O (2 mL). The mixture was stirred at 20° C. for 12 hrs. The reaction was adjusted to pH=3-4 with HCl (1 M aq) and concentrated under reduced pressure to give compound 90-6 (600 mg, 1.20 mmol, 89.6% yield, 91.5% purity) as a yellow solid.

LCMS: RT=0.778 min. m/z=458.0 (M+1)⁺.

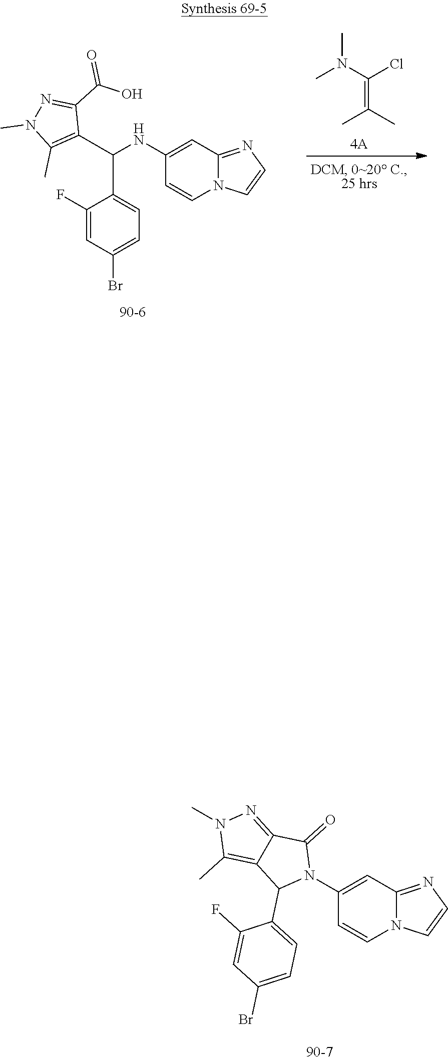

To a solution of compound 90-6 (550 mg, 1.10 mmol, 91.5% purity, 1.00 eq) in DCM (2 mL) was added compound 4A (303 mg, 2.27 mmol, 300 μL, 2.07 eq) at 0° C. The mixture was stirred at 0° C. for 1 hr and 20° C. for 24 hrs. The reaction mixture was partitioned between water 150 mL and DCM 150 mL. The organic phase was separated, washed with brine 100 ml, dried over Na₂SO₄, filtered and concentrated under reduced pressure to give a residue. The crude product was purified by reversed-phase HPLC (0.1% NH₃.H₂O condition in acetonitrile) to give compound 90-7 (200 mg, 454.27 μmol, 41.37% yield) as a white solid.

¹H NMR: 400 MHz DMSO-d6 δ 8.47-8.45 (m, 1H), 7.82 (s, 1H), 7.59-7.58 (m, 1H), 7.49-7.48 (m, 1H), 7.33-7.32 (m, 1H), 7.30-7.29 (m, 3H), 6.72 (s, 1H), 3.87 (s, 3H), 2.13 (s, 3H).

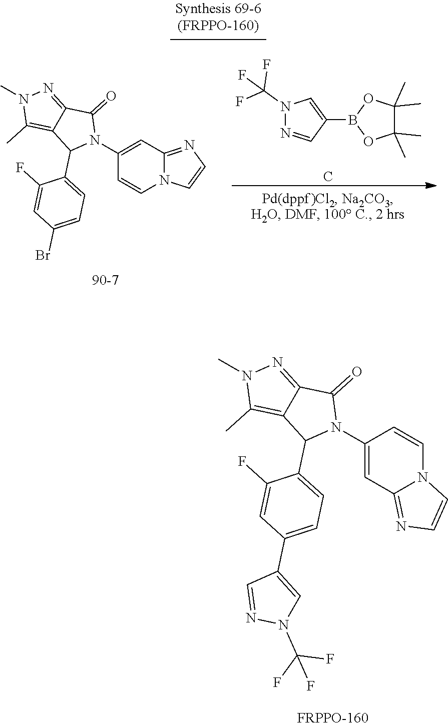

To a solution of compound 90-7 (50 mg, 113 μmol, 1.00 eq) and compound C (89 mg, 339 μmol, 2.99 eq) in DMF (2.0 mL) was and H₂O (0.2 mL) added Na₂CO₃ (36 mg, 340 μmol, 3.00 eq) and Pd(dppf)Cl₂ (41 mg, 56.0 μmol, 0.50 eq). The mixture was stirred at 100° C. for 2 hrs. The reaction mixture was partitioned between water 100 mL and Ethyl acetate 150 mL. The organic phase was separated, washed with water 200 mL (100 mL*2), dried over Na₂SO₄, filtered and concentrated under reduced pressure to give a residue. The residue was purified by prep-TLC (SiO₂, DCM:MeOH=10:1, product R_{f}=0.5) to give FRPPO-160 (22.11 mg, 41.1 μmol, 36.1% yield, 92% purity) as a yellow oil.

LCMS: RT=0.936 min, m/z=496.3 (M+1)⁺.

HPLC: RT=1.955 min.

¹H NMR: 400 MHz CDCl₃ δ 8.06-8.04 (m, 1H), 7.97-7.93 (m, 2H), 7.84 (s, 1H), 7.52-7.24 (m, 3H), 7.22 (s, 1H), 7.15-7.14 (m, 1H), 7.13-7.12 (m, 2H), 6.43 (s, 1H), 3.92 (s, 3H), 2.21 (s, 3H).

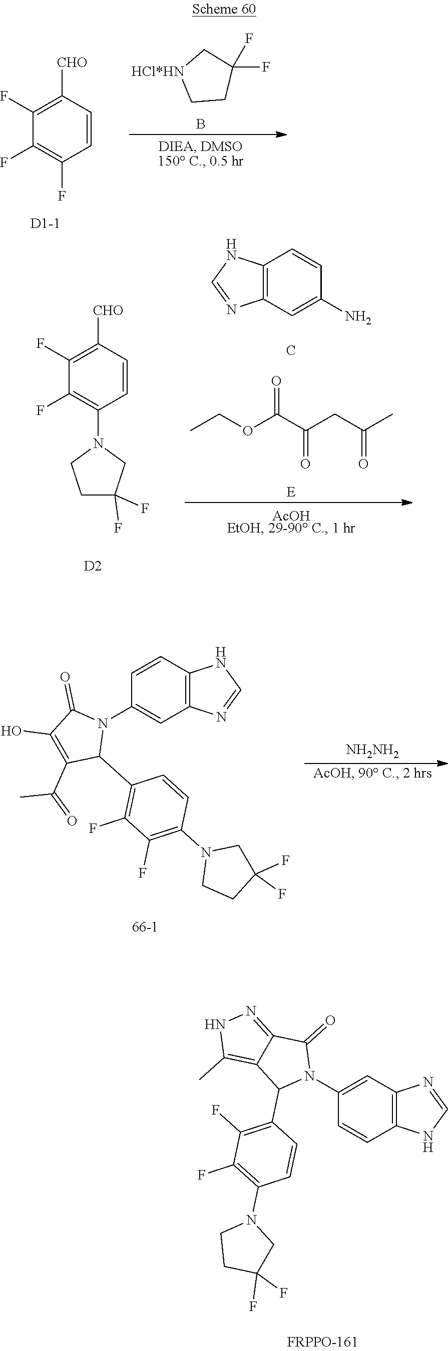

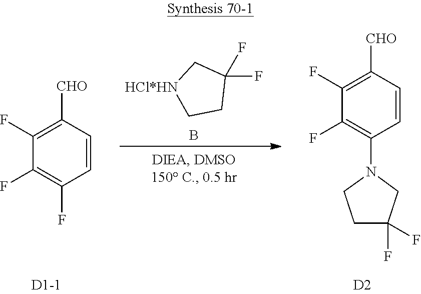

To a solution of compound D1-1 (1.00 g, 6.25 mmol, 1.00 eq) in DMSO (5 mL) was added DIEA (1.61 g, 12.4 mmol, 2.18 mL, 2.00 eq) and compound B (900 mg, 6.27 mmol, 1.00 eq, HCl). The mixture was stirred at 150° C. for 0.5 hr. The mixture was added to water 100 mL and extracted with Ethyl acetate (150 mL), the combined organic phase was washed with water (100 mL*2) and brine 100 mL, dried over Na₂SO₄, filtered and concentrated under reduced pressure to give a residue. The residue was purified by column chromatography (SiO₂, petroleum ether:ethyl acetate=1:0 to 10:1, product R_{f}=0.5) to give compound D2 (650 mg, 2.63 mmol, 42.1% yield) as a yellow solid.

¹H NMR: 400 MHz DMSO-d6 δ 10.1 (s, 1H), 7.55-7.51 (m, 1H), 6.44-6.40 (m, 1H), 4.00-3.93 (m, 2H), 3.79-3.68 (m, 2H), 2.54-2.40 (m, 2H).

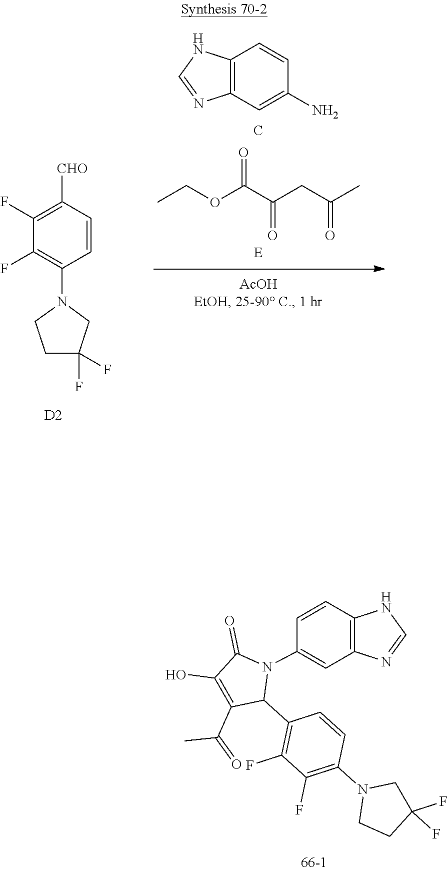

To a solution of compound D2 (409 mg, 1.65 mmol, 1.02 eq) in EtOH (10 mL) was added AcOH (97 mg, 1.62 mmol, 92.3 μL, 1.00 eq) and compound C (215 mg, 1.61 mmol, 1.00 eq). The mixture was stirred at 25° C. for 0.5 hr. Then ethyl compound E (260 mg, 1.64 mmol, 232 μL, 1.02 eq) was added, the mixture was stirred at 90° C. for 0.5 hr. The mixture was added to NaHCO₃ 100 mL and extracted with Ethyl acetate (150 mL), the combined organic phase was washed with water (100 mL*2) and brine 100 mL, dried over Na₂SO₄, filtered and concentrated under reduced pressure to give a residue. The crude product was purified by reversed-phase HPLC (0.1% NH₃.H₂O in acetonitrile) to give compound 66-1 (30 mg, 58.2 μmol, 3.61% yield, 92.1% purity) as a brown solid.

¹H NMR: 400 MHz DMSO-d6 δ 8.12 (s, 1H), 7.67 (s, 1H), 7.46-7.44 (m, 1H), 7.29-7.27 (m, 1H), 6.66 (s, 1H), 6.39-6.35 (m, 1H), 5.97-5.92 (m, 1H), 3.66-3.57 (m, 2H), 2.42-2.37 (m, 2H), 2.35-2.13 (m, 2H), 1.62 (s, 3H).

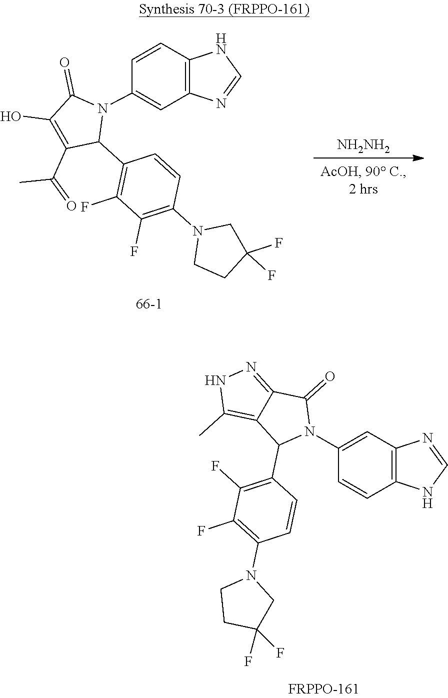 [SMILES: CC(=O)C1=C(O)C(=O)N(c2ccc3[nH]cnc3c2)C1c1ccc(N2CCC(F)(F)C2)c(F)c1F ; "NH2NH2" ; Cc1[nH]nc2c1C(c1ccc(N3CCC(F)(F)C3)c(F)c1F)N(c1ccc3[nH]cnc3c1)C2=O]

To a solution of compound 66-1 (30 mg, 58.2 μmol, 1.00 eq) in AcOH (10 mL) was added N₂H₄—H₂O (0.14 g, 2.38 mmol, 135 μL, 40.8 eq). The mixture was stirred at 90° C. for 2 hr. The reaction mixture was concentrated under reduced pressure to remove AcOH. The residue was diluted with water 100 mL and adjusted to pH=8-9 with NH₄OH, then extracted with Ethyl acetate 150 mL (50 mL*3). The combined organic layers were washed with brine 100 mL, dried over Na₂SO₄, filtered and concentrated under reduced pressure to give a residue. The residue was purified by prep-HPLC (basic condition column: Waters Xbridge 150*25 mm*5 μm; mobile phase: 0.05% ammonia hydroxide, A-water, B-ACN; gradient: 23%-53% B %, 10 min) to give FRPPO-161 (5 mg, 10.5 μmol, 18.1% yield, 99.2% purity) as a white solid.

LCMS: RT=0.906 min, m/z=471.3 (M+1)⁺.

HPLC: RT=1.776 min

¹H NMR: 400 MHz DMSO-d6 δ 8.19 (s, 1H), 7.71-7.59 (m, 1H), 7.53-7.51 (m, 1H), 7.31-7.29 (m, 1H), 7.22-7.20 (m, 1H), 6.52 (s, 1H), 6.47-6.43 (m, 1H), 3.74-3.67 (m, 2H), 3.50-3.46 (m, 2H), 2.43-2.39 (m, 2H), 2.09 (s, 3H).

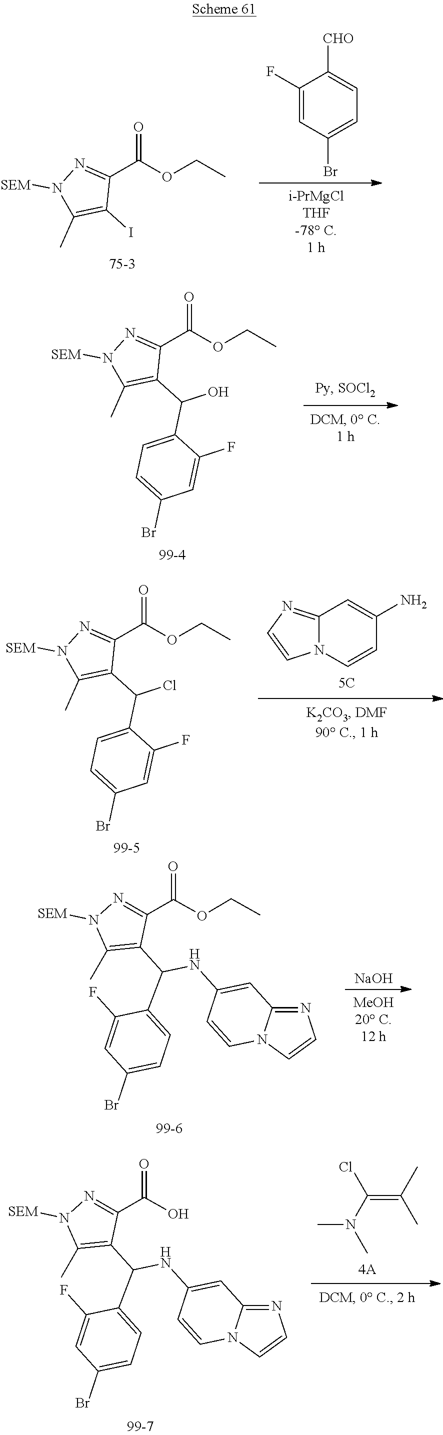

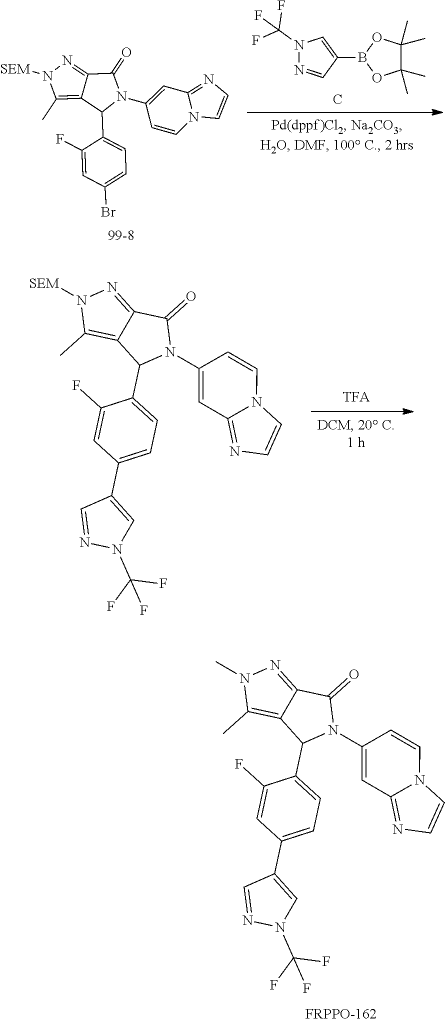

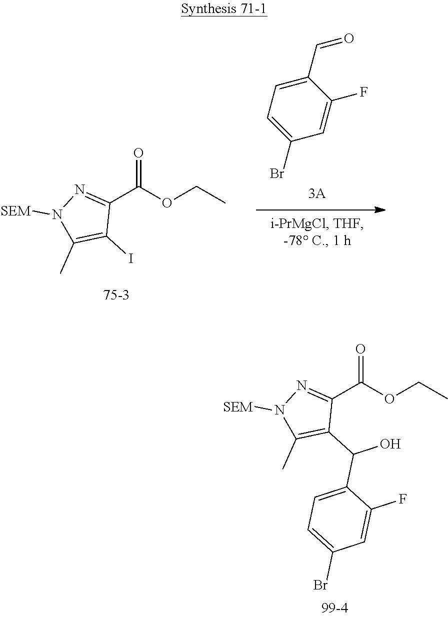 [SMILES: CCOC(=O)c1nn(COCC[Si](C)(C)C)c(C)c1I ; O=Cc1ccc(Br)cc1F ; CCOC(=O)c1nn(COCC[Si](C)(C)C)c(C)c1C(O)c1ccc(Br)cc1F]

To a solution of compound 75-3 (Scheme 58 and Synthesis 68-2) (4.00 g, 9.75 mmol, 1.00 eq) in THF (10 mL) was added i-PrMgCl (2 M, 5.5 mL, 1.13 eq), the mixture was stirred at −78° C. for 0.5 hr, then compound 3A (2.40 g, 11.8 mmol, 1.21 eq) in THF (2 mL) was added, the mixture was stirred at −78° C. for 0.5 hr. The reaction mixture was quenched by addition water (20 mL), and then diluted with water 100 mL and extracted with Ethyl acetate 300 mL (100 mL*3). The combined organic layers were washed with brine 150 mL, dried over Na₂SO₄, filtered and concentrated under reduced pressure to give a residue. The residue was purified by flash silica gel chromatography (ISCO®; 80 g SepaFlash® Silica Flash Column, Eluent of 0-30% ethyl acetate:petroleum ether gradient @ 60 mL/min), (TLC petroleum ether:ethyl acetate=3:1, product R_{f}=0.3) to give compound 99-4 (2.50 g, 5.13 mmol, 52.6% yield) as a colorless oil.

¹H NMR: 400 MHz CDCl₃ δ 7.45-7.41 (m, 1H), 7.28-7.27 (m, 1H), 7.26-7.14 (m, 1H), 5.73-5.71 (m, 1H), 5.65-5.63 (m, 1H), 4.38-4.35 (m, 2H), 3.74-3.71 (m, 2H), 3.55-3.51 (m, 2H), 3.22 (s, 3H), 1.37-1.34 (m, 3H), 0.87-0.81 (m, 2H), −0.05 (s, 9H).

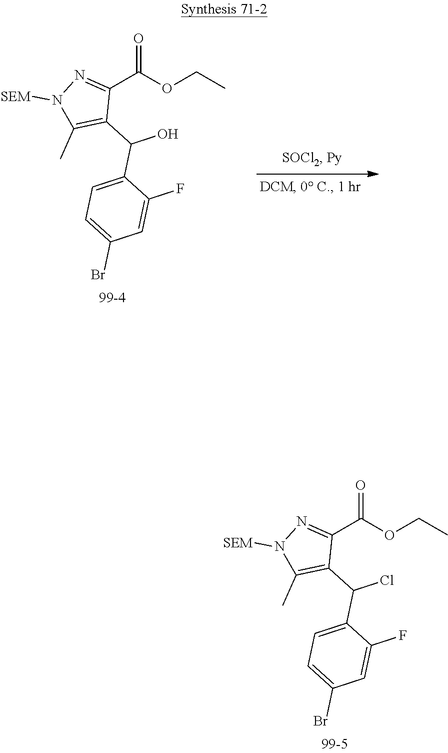 [SMILES: CCOC(=O)c1nn(COCC[Si](C)(C)C)c(C)c1C(O)c1ccc(Br)cc1F ; CCOC(=O)c1nn(COCC[Si](C)(C)C)c(C)c1C(Cl)c1ccc(Br)cc1F]

To a solution of compound 99-4 (1.40 g, 2.87 mmol, 1.00 eq) in DCM (20 mL) was added Py (686 mg, 8.67 mmol, 700 μL, 3.02 eq) and SOCl₂ (459 mg, 3.86 mmol, 280 μL, 1.34 eq) at 0° C. The mixture was stirred at 0° C. for 1 hr. TLC (petroleum ether:ethyl acetate=1:1) showed compound 99-4 (R_{f}=0.5) was consumed, and new spot (R_{f}=0.6) formed. The reaction mixture was concentrated under reduced pressure to give compound 99-5 (1.45 g, crude) as a yellow solid.

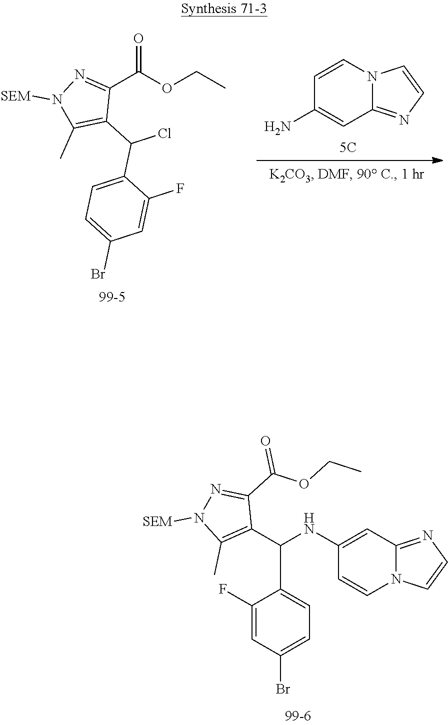 [SMILES: CCOC(=O)c1nn(COCC[Si](C)(C)C)c(C)c1C(Cl)c1ccc(Br)cc1F ; Nc1ccn2ccnc2c1 ; CCOC(=O)c1nn(COCC[Si](C)(C)C)c(C)c1C(Nc1ccn2ccnc2c1)c1ccc(Br)cc1F]

To a solution of compound 99-5 (1.45 g, 2.87 mmol, 1.00 eq) in DMF (10 mL) was added K₂CO₃ (1.19 g, 8.60 mmol, 3.00 eq) and compound 5C (580 mg, 4.36 mmol, 1.52 eq). The mixture was stirred at 90° C. for 1 hr. The reaction mixture was partitioned between water (100 mL) and Ethyl acetate (150 mL). The organic phase was separated, washed with brine (150 mL), dried over Na₂SO₄, filtered and concentrated under reduced pressure to give a residue. The crude product was purified by reversed-phase HPLC (0.1% HCl condition in acetonitrile) to give compound 99-6 (800 mg, 1.33 mmol, 46.3% yield, 100% purity) as a yellow solid.

¹H NMR: 400 MHz CDCl₃ δ 7.86-7.83 (m, 1H), 7.41-7.40 (m, 1H), 7.40-7.39 (m, 1H), 7.35-7.34 (m, 1H), 7.28-7.27 (m, 1H), 7.18-7.15 (m, 1H), 6.45 (s, 1H), 6.39-6.37 (m, 2H), 6.01-5.99 (m, 1H), 5.48-5.46 (m, 2H), 4.44-4.35 (m, 2H), 3.57-3.53 (m, 2H), 2.47 (s, 3H), 1.38-1.34 (m, 3H), 0.88-0.84 (m, 2H), −0.05-−0.06 (m, 9H).

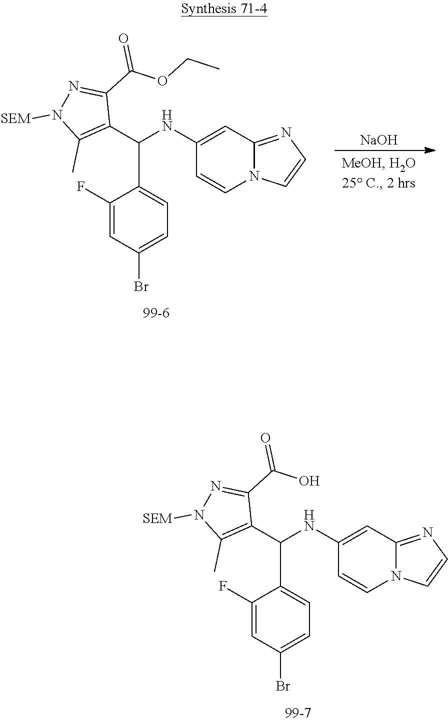 [SMILES: CCOC(=O)c1nn(COCC[Si](C)(C)C)c(C)c1C(Nc1ccn2ccnc2c1)c1ccc(Br)cc1F ; "NaOH" ; Cc1c(C(Nc2ccn3ccnc3c2)c2ccc(Br)cc2F)c(C(=O)O)nn1COCC[Si](C)(C)C]

To a solution of compound 99-6 (800 mg, 1.33 mmol, 1.00 eq) in MeOH (6 mL) was added NaOH (800 mg, 20.0 mmol, 15.0 eq) in H₂O (3 mL). The mixture was stirred at 25° C. for 2 hrs. The reaction mixture was concentrated under reduced pressure to remove MeOH. The residue was adjusted to pH=3-4 with HCl (1M) and extracted with Ethyl acetate 150 mL (50 mL*3). The combined organic layers were washed with brine (100 mL), dried over Na₂SO₄, filtered and concentrated under reduced pressure to give compound 99-7 (750 mg, 1.20 mmol, 90.5% yield, 92.1% purity) as a yellow solid.

LCMS: RT=0.799 min, m/z=576.1 (M+1)⁺.

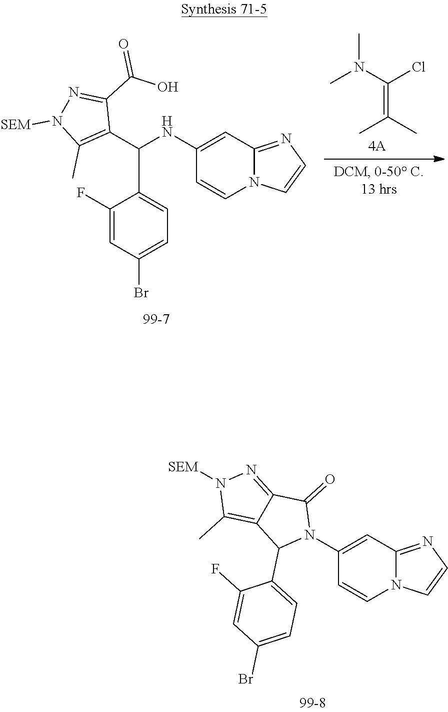

To a solution of compound 99-7 (650 mg, 1.04 mmol, 92.1% purity, 1.00 eq) in DCM (5 mL) was added compound 4A (328 mg, 2.46 mmol, 325 μL, 2.36 eq) at 0° C. The mixture was stirred at 20° C. for 1 hr and 50° C. for 12 hrs. The reaction mixture was concentrated under reduced pressure to give a residue. The crude product was purified by reversed-phase HPLC (0.1% NH₃.H₂O condition in acetonitrile) to give compound 99-8 (300 mg, 522 μmol, 50.1% yield, 96.9% purity) as a yellow solid.

¹H NMR: 400 MHz DMSO-d6 δ 8.07-8.05 (m, 1H), 7.75-7.73 (m, 1H), 7.56 (s, 1H), 7.49 (s, 1H), 7.32-7.31 (m, 2H), 7.29-7.15 (m, 1H), 6.93-6.91 (m, 1H), 6.41 (s, 1H), 3.63-3.56 (m, 2H), 2.28 (s, 3H), 0.91-0.87 (m, 2H), −0.02-−0.04 (m, 9H).

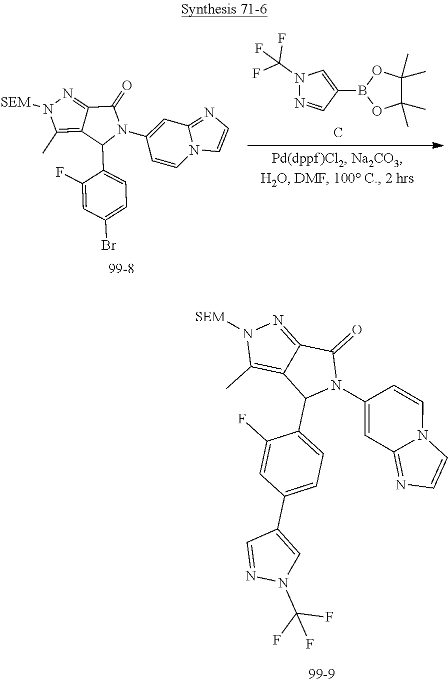

To a solution of compound 99-8 (200 mg, 348 μmol, 96.9% purity, 1.00 eq) and compound C (100 mg, 381 μmol, 1.10 eq) in DMF (2.5 mL) was and H₂O (0.5 mL) added Na₂CO₃ (120 mg, 1.13 mmol, 3.25 eq) and Pd(dppf)Cl₂ (30 mg, 41.0 μmol, 0.10 eq). The mixture was stirred at 100° C. for 2 hr. The reaction mixture was partitioned between water 100 mL and Ethyl acetate 150 mL. The organic phase was separated, washed with water 200 mL (100 mL*2), dried over Na₂SO₄, filtered and concentrated under reduced pressure to give a residue. The crude product was purified by reversed-phase HPLC (0.1% NH₃.H₂O in acetonitrile) to give compound 99-9 (100 mg, 162 μmol, 46.6% yield, 99.3% purity) as a yellow solid.

LCMS: RT=1.055 min, m/z=612.2 (M+1)⁺.

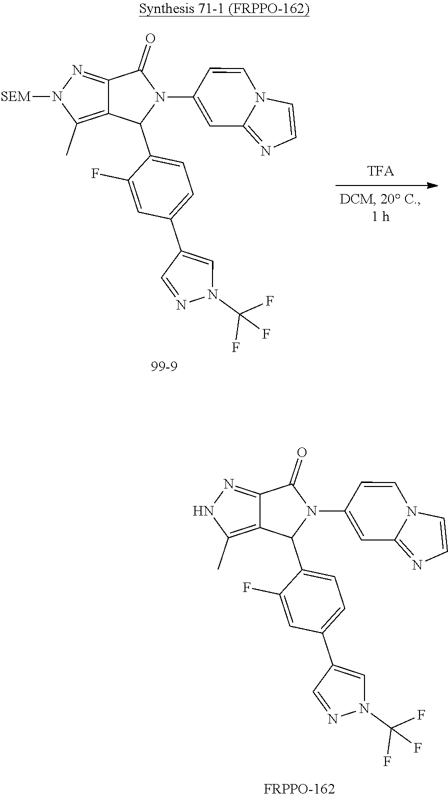 [SMILES: Cc1c2c(nn1COCC[Si](C)(C)C)C(=O)N(c1ccn3ccnc3c1)C2c1ccc(-c2cnn(C(F)(F)F)c2)cc1F ; Cc1[nH]nc2c1C(c1ccc(-c3cnn(C(F)(F)F)c3)cc1F)N(c1ccn3ccnc3c1)C2=O]

To a solution of compound 99-9 (100 mg, 162 μmol, 99.3% purity, 1.00 eq) in DCM (1.5 mL) was added TFA (770 mg, 6.75 mmol, 0.5 mL, 41.6 eq). The mixture was stirred at 20° C. for 1 hr. The reaction mixture was concentrated under reduced pressure to remove DCM. The residue was diluted with water 100 mL and adjusted to pH=8-9 with NH₃.H₂O, then extracted with DCM 150 mL (50 mL*3). The combined organic layers were washed with brine (100 mL), dried over Na₂SO₄, filtered and concentrated under reduced pressure to give a residue. The residue was purified by prep-HPLC (basic condition column: Welch Xtimate C18 150*30 mm*5 μm; mobile phase: [water (0.05% ammonia hydroxide v/v)-ACN]; B %: 22%-52%, 11.5 min) to give FRPPO-162 (50.47 mg, 99.0 μmol, 61.0% yield, 94.5% purity) as a yellow solid.

LCMS: RT=0.875 min, m/z=482.2 (M+1)⁺.

HPLC: RT=2.154 min.

¹H NMR: 400 MHz CDCl₃ δ 8.08-8.06 (m, 1H), 7.98 (s, 1H), 7.94 (s, 1H), 7.56 (s, 1H), 7.49 (s, 1H), 7.41 (s, 1H), 7.27-7.24 (m, 1H), 7.24 (s, 1H), 7.14-7.13 (m, 1H), 7.10-7.08 (m, 1H), 6.47 (s, 1H), 2.29 (s, 3H).

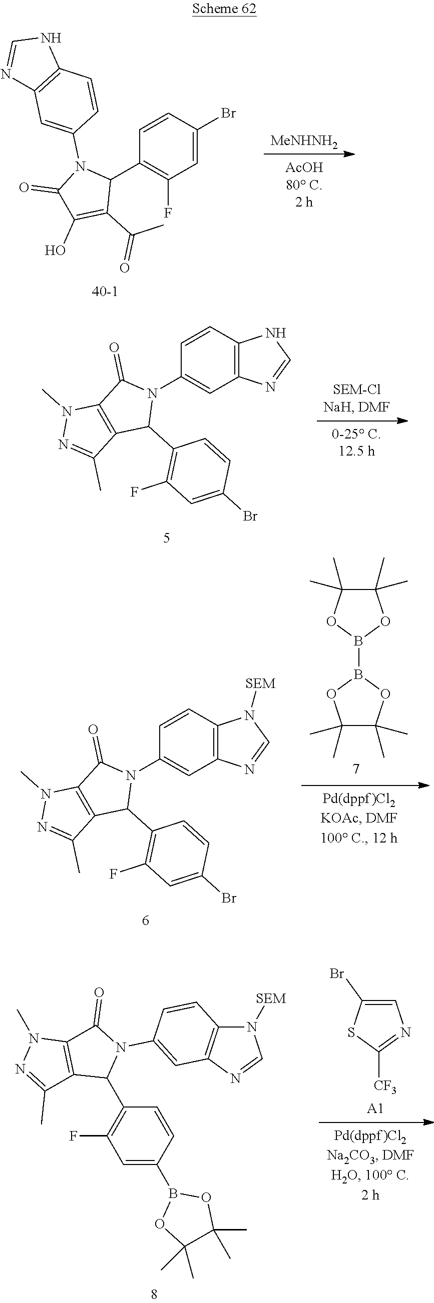

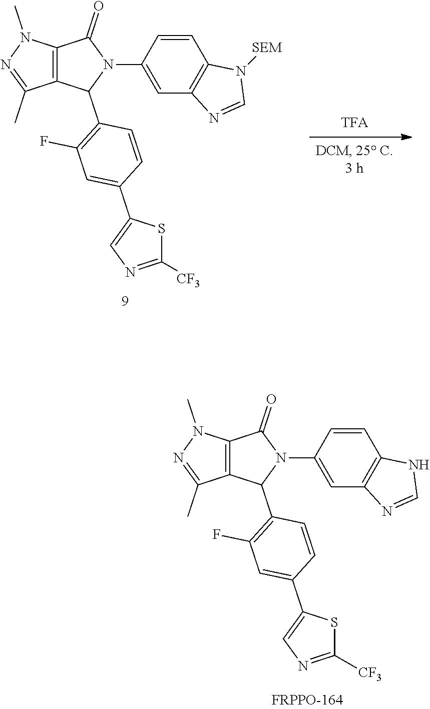 [SMILES: Cc1nn(C)c2c1C(c1ccc(-c3cnc(C(F)(F)F)s3)cc1F)N(c1ccc3c(c1)ncn3COCC[Si](C)(C)C)C2=O ; Cc1nn(C)c2c1C(c1ccc(-c3cnc(C(F)(F)F)s3)cc1F)N(c1ccc3[nH]cnc3c1)C2=O]

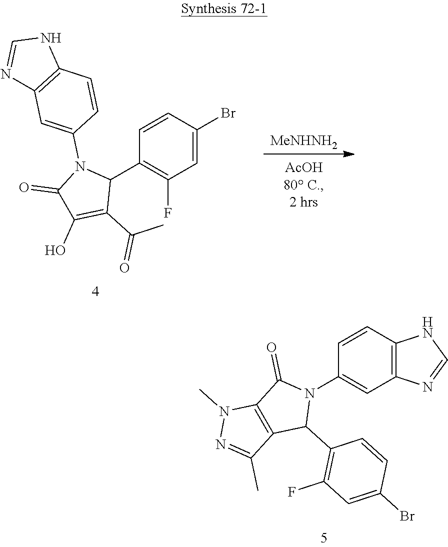 [SMILES: "MeNHNH2" ; CC(=O)C1=C(O)C(=O)N(c2ccc3[nH]cnc3c2)C1c1ccc(Br)cc1F ; Cc1nn(C)c2c1C(c1ccc(Br)cc1F)N(c1ccc3[nH]cnc3c1)C2=O]

To a solution of compound 40-1 (from Scheme 42 and Synthesis 46-1) (9.70 g, 22.6 mmol, 1.00 eq) in AcOH (50 mL) was added methylhydrazine (2.60 g, 22.6 mmol, 2.97 mL, 40.0% purity, 1.00 eq). The mixture was stirred at 80° C. for 2 hrs. The reaction mixture was drop-wise add into water and adjust to pH=8-9, filtered and concentrated under reduced pressure to give a residue. The residue was purified by prep-HPLC (basic condition; column: Xtimate C18 10u 250 mm*80 mm; mobile phase: [water (0.05% NH₃.H₂O+10 mM NH₄HCO₃)-ACN]; B %: 20%-40%, 25 min) to give compound 5 (2.40 g, 5.45 mmol, 48.4% yield) as yellow solid.

¹H NMR: 400 MHz, DMSO-d6 δ 12.48 (s, 1H), 8.20 (s, 1H), 7.71-7.47 (m, 3H), 7.30-7.26 (m, 2H), 6.58 (br d, J=5.9 Hz, 1H), 3.96 (s, 3H), 1.99 (s, 3H).

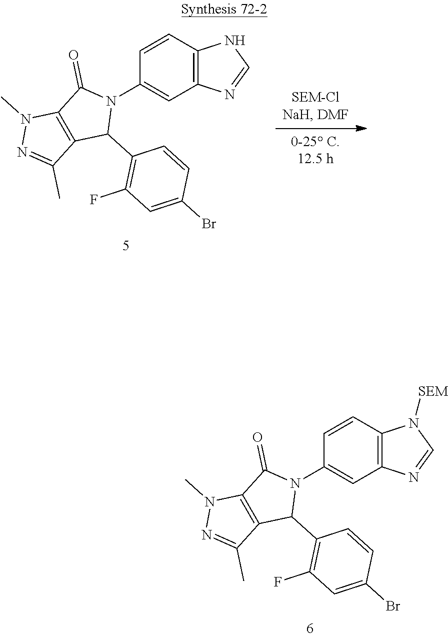 [SMILES: Cc1nn(C)c2c1C(c1ccc(Br)cc1F)N(c1ccc3[nH]cnc3c1)C2=O ; Cc1nn(C)c2c1C(c1ccc(Br)cc1F)N(c1ccc3c(c1)ncn3COCC[Si](C)(C)C)C2=O]

To a solution of compound 5 (800 mg, 1.82 mmol, 1.00 eq) in DMF (10 mL) was added NaH (88 mg, 2.20 mmol, 60% purity, 1.21 eq) at 0° C., the mixture was stirred at 0° C. for 0.5 hr, then SEM-Cl (303 mg, 1.82 mmol, 321 μL, 1.00 eq) was added to the mixture and the mixture was stirred at 25° C. for 12 hrs. The mixture was quenched with water (50 mL) and extracted with EtOAc (30 mL*2). The combined organic layers were dried over anhydrous sodium sulfate, filtrated and concentrated to give a residue. The crude product was purified by reversed-phase HPLC (0.1% NH₃.H₂O in acetonitrile) to get compound 6 (480 mg, 841 μmol, 46.3% yield, 100% purity) as a yellow oil.

LCMS: RT=1.077 min. m/z=570.1 (M+1)⁺.

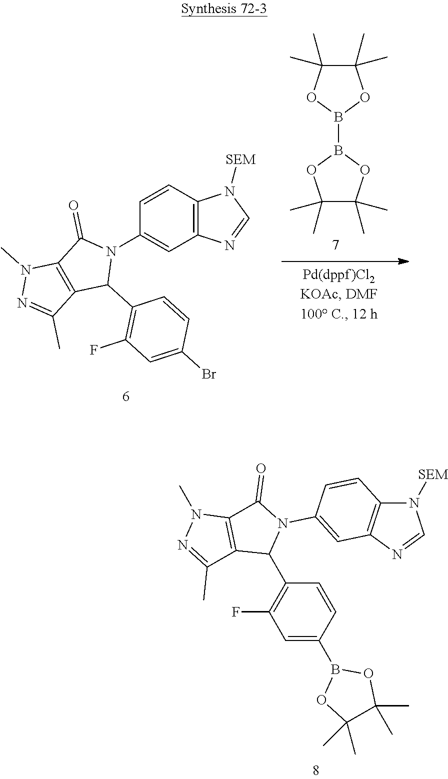

To a solution of compound 6 (380 mg, 666 μmol, 100% purity, 1.00 eq) and compound 7 (300 mg, 1.18 mmol, 1.77 eq) in DMF (5 mL) was added KOAc (200 mg, 2.04 mmol, 3.06 eq) and Pd(dppf)Cl₂ (40 mg, 54.6 μmol, 0.08 eq). The mixture was stirred at 100° C. for 15 hrs. LCMS showed desired mass (RT=0.948 min, m/z=618.1) was detected. The mixture was filtrated and the filtrate was diluted with EtOAc (20 mL) and washed with water (20 mL), brine (20 mL), the organic layer was dried over anhydrous sodium sulfate, filtrated and concentrated to give compound 8 (600 mg, crude) as a black oil and used in the next step without purification.

LCMS: RT=0.948 min, m/z=618.1 (M+1)⁺.

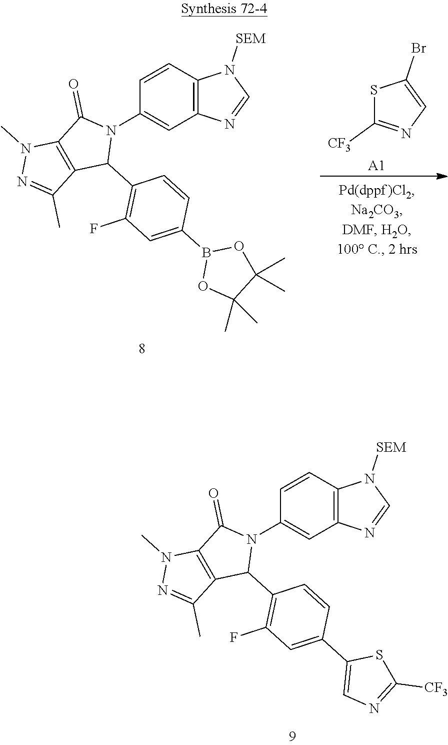

To a solution of compound 8 (540 mg, 874 μmol, 2.25 eq) and compound A1 (90 mg, 388 μmol, 1.00 eq) in DMF (5 mL) and H₂O (0.5 mL) was added Na₂CO₃ (135 mg, 1.27 mmol, 3.28 eq) and Pd(dppf)Cl₂ (90 mg, 123 μmol, 0.300 eq). The mixture was stirred at 100° C. for 2 hrs. LCMS (EW23535-6-P1A) showed desired mass (RT=1.002 min, m/z=642.2) was detected. The mixture was filtrated and the filtrate was washed with water (10 mL), brine (10 mL), dried over anhydrous sodium sulfate, filtrated and concentrated to give compound 9 (100 mg, crude) as a black oil and used in the next step without purification.

LCMS: RT=1.002 min, m/z=642.2 (M+1)⁺.

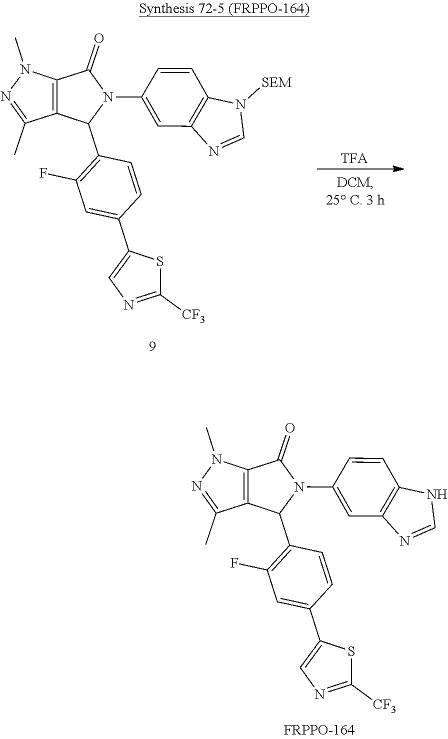 [SMILES: Cc1nn(C)c2c1C(c1ccc(-c3cnc(C(F)(F)F)s3)cc1F)N(c1ccc3c(c1)ncn3COCC[Si](C)(C)C)C2=O ; Cc1nn(C)c2c1C(c1ccc(-c3cnc(C(F)(F)F)s3)cc1F)N(c1ccc3[nH]cnc3c1)C2=O ; "TFA"]

To a solution of compound 9 (100 mg, 156 μmol, 1.00 eq) in DCM (1 mL) was added TFA (1.54 g, 13.5 mmol, 1 mL, 86.8 eq). The mixture was stirred at 25° C. for 12 hrs. The mixture was dilute with DCM (20 mL) and adjusted to pH=8 with Na₂CO₃ solution, the mixture was extracted with DCM (20 mL), dried over anhydrous sodium sulfate, filtrated and concentrated to give a residue. The crude product was purified by reversed-phase HPLC (0.1% NH₃.H₂O in acetonitrile) to get FRPPO-164 (24.16 mg, 43.2 μmol, 27.8% yield, 91.6% purity) as an off-white solid.

LCMS: RT=0.913 min, m/z=513.1 (M+1)⁺.

HPLC: RT=2.307 min.

¹H NMR: 400 MHz CDCl₃ δ 10.28 (s, 1H), 8.00 (s, 1H), 7.90 (s, 1H), 7.72 (s, 1H), 7.25-7.14 (m, 5H), 6.37 (s, 1H), 4.09 (s, 3H), 2.14 (s, 3H).

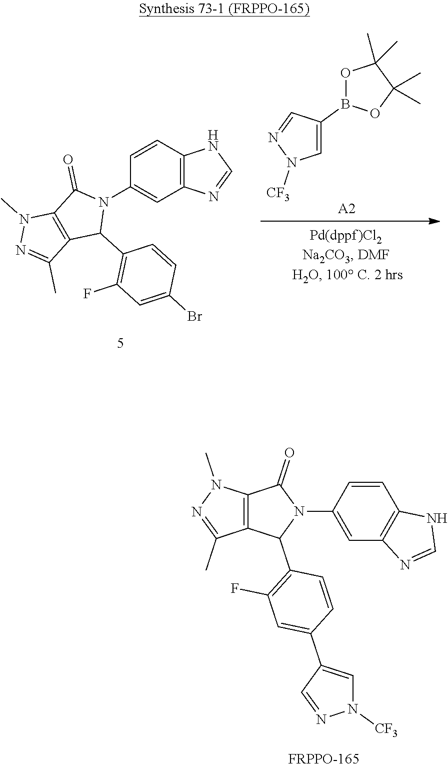 [SMILES: Cc1nn(C)c2c1C(c1ccc(Br)cc1F)N(c1ccc3[nH]cnc3c1)C2=O ; CC1(C)OB(c2cnn(C(F)(F)F)c2)OC1(C)C ; "A2" ; Cc1nn(C)c2c1C(c1ccc(-c3cnn(C(F)(F)F)c3)cc1F)N(c1ccc3[nH]cnc3c1)C2=O]

To a solution of compound 5 (Scheme 62 and Synthesis 72-1) (20 mg, 45.43 μmol, 1.00 eq) and compound A2 (15 mg, 57.2 μmol, 1.26 eq) in DMF (1 mL) and H₂O (0.1 mL) was added Pd(dppf)Cl₂ (7 mg, 9.57 μmol, 2.11e-1 eq) and Na₂CO₃ (15 mg, 141 μmol, 3.12 eq). The mixture was stirred at 100° C. for 2 hrs under N₂. The mixture was filtrated and the filtrate was concentrated to give a residue. The residue was purified by prep-HPLC (basic condition: column: Waters Xbridge Prep OBD C18 150*40 mm*10 μm; mobile phase: [water (0.05% NH₃.H₂O+10 mM NH₄HCO₃)-ACN]; B %: 20%-60%, 8 min) to give compound FRPPO-165 (4.50 mg, 8.84 μmol, 19.5% yield, 97.3% purity) as an off-white solid.

LCMS: RT=0.543 min m/z=496.4 (M+1)⁺.

HPLC: RT=2.234 min.

¹H NMR: 400 MHz DMSO-d6 δ 12.47 (br s, 1H), 8.95 (s, 1H), 8.41 (s, 1H), 8.18 (d, J=3.9 Hz, 1H), 7.70 (br d, J=18.9 Hz, 1H), 7.59-7.43 (m, 3H), 7.39-7.24 (m, 2H), 6.60 (d, J=10.5 Hz, 1H), 3.97 (s, 3H), 2.00 (s, 3H).

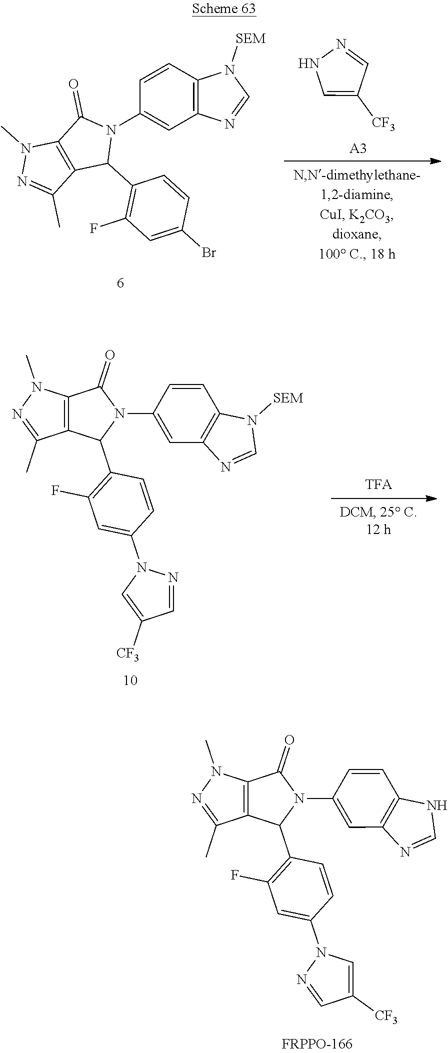

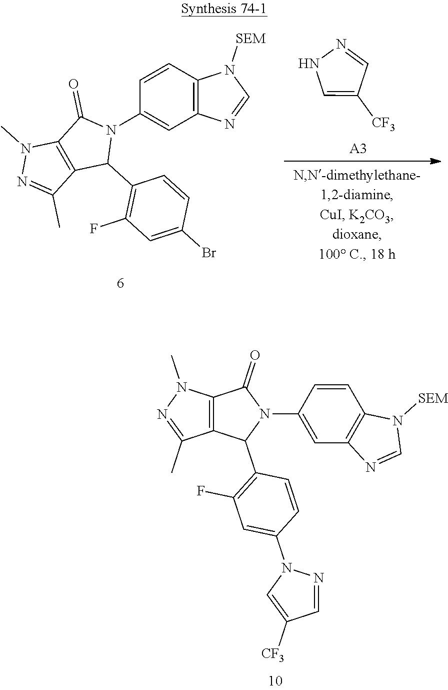

A mixture of compound 6 (Scheme 62 and Synthesis 72-2) (50 mg, 87.7 μmol, 1.00 eq), compound A3 (24 mg, 176 μmol, 2.01 eq), CuI (17 mg, 89.3 μmol, 1.02 eq) N,N′-dimethylethylenediamine (16 mg, 181 μmol, 19.6 μL, 2.07 eq) K₂CO₃ (25 mg, 181 μmol, 2.06 eq) and dioxane (2 mL) was stirred at 120° C. for 18 hrs under N₂. The mixture was filtrated and the filtrate was concentrated to give a residue. The residue was purified by prep-HPLC (column: 3_Phenomenex Luna C18 75*30 mm*3 μm; mobile phase: [water (0.2% FA)-ACN]; B %: 50%-80%, 9 min) to give compound 10 (20 mg, 26.0 μmol, 29.7% yield, 81.4% purity) as yellow oil.

LCMS: RT=1.081 min m/z=626.4 (M+1)⁺.

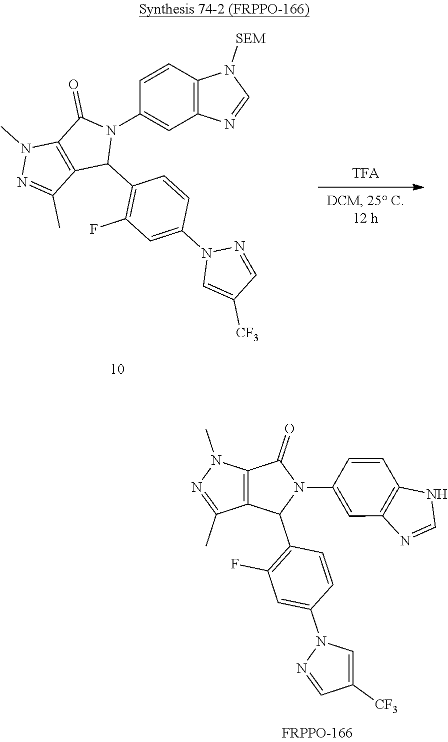 [SMILES: Cc1nn(C)c2c1C(c1ccc(-n3cc(C(F)(F)F)cn3)cc1F)N(c1ccc3c(c1)ncn3COCC[Si](C)(C)C)C2=O ; Cc1nn(C)c2c1C(c1ccc(-n3cc(C(F)(F)F)cn3)cc1F)N(c1ccc3[nH]cnc3c1)C2=O]

To a solution of compound 10 (15 mg, 24.0 μmol, 1.00 eq) in DCM (5 mL) was added TFA (1.54 g, 13.51 mmol, 1 mL, 564 eq) and the mixture was stirred at 25° C. for 12 hrs. The mixture was diluted with DCM (20 mL) and adjusted to pH=8 with Na₂CO₃ solution, extracted with DCM (10 mL*2) dried over anhydrous sodium sulfate, filtrated and concentrated to a white oil (900 mg, crude) which was used to be purified by prep-HPLC (column: Waters Xbridge BEH C18 250*50 mm*10 μm; mobile phase: [water (0.05% ammonia hydroxide v/v)-ACN]; B %: 35%-55%, 15 min) to give FRPPO-166 (7.69 mg, 15.3 μmol, 0.85% yield, 98.7% purity) as a yellow solid.

LCMS: RT=0.909 min, m/z=496.2 (M+1)⁺.

HPLC: RT=2.304 min.

¹H NMR: 400 MHz CDCl_{3 δ 8.10} (s, 1H), 8.01 (s, 1H), 7.86 (s, 1H), 7.75 (s, 1H), 7.47-7.44 (m, 1H), 7.33-7.30 (m, 3H), 7.19-7.17 (m, 1H), 6.39 (s, 1H), 4.10 (s, 3H), 2.14 (s, 3H).

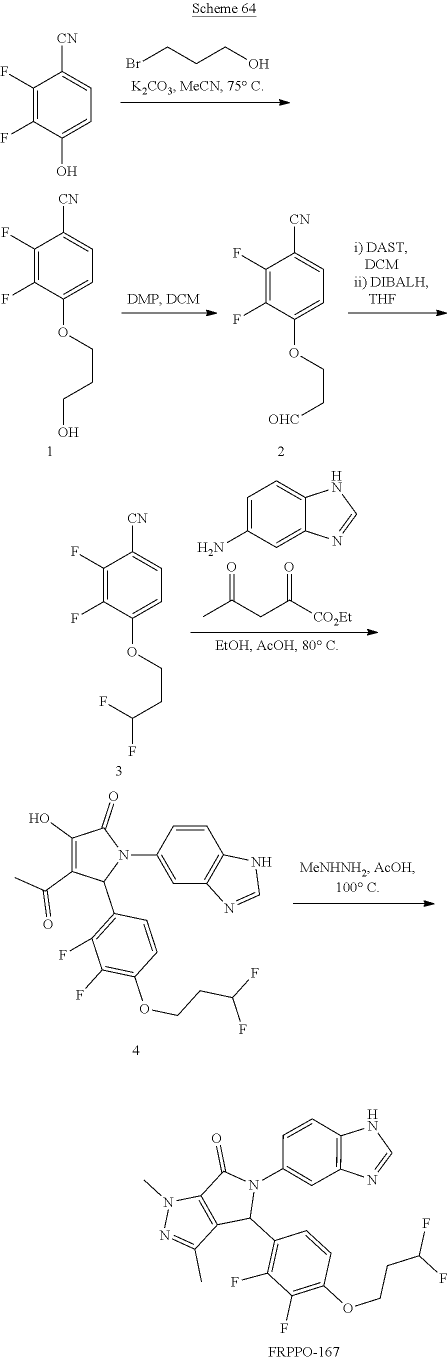

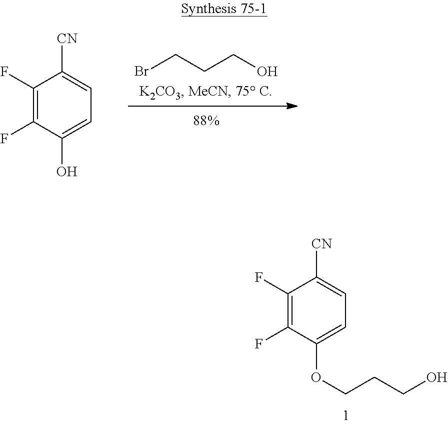 [SMILES: N#Cc1ccc(O)c(F)c1F ; OCCCBr ; N#Cc1ccc(OCCCO)c(F)c1F]

To a solution of 2,3-difluoro-4-hydroxybenzonitrile (5.0 g, 32.8 mmol) in acetonitrile (40 mL) was added 3-bromopropan-1-ol (5.82 g, 1.3 eq.) and potassium carbonate (8.91 g, 2.0 eq.) and the reaction was heated at 75° C. overnight. The reaction was diluted with brine and extracted with ethyl acetate. The combined organic layers were dried over sodium sulfate and concentrated in vacuo. The crude product was purified by silica gel chromatography to give the product 1 as an orange oil (6.17 g, 88%).

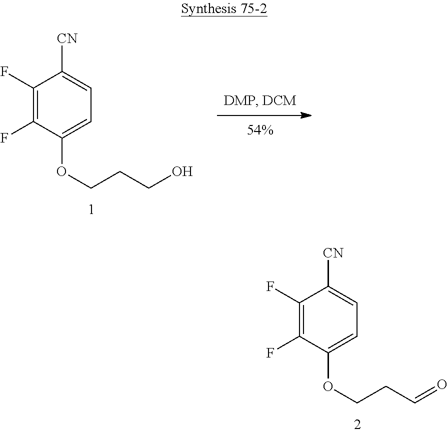 [SMILES: N#Cc1ccc(OCCCO)c(F)c1F ; N#Cc1ccc(OCCC=O)c(F)c1F]

To a solution of 2,3-difluoro-4-(3-hydroxypropoxy)benzonitrile 1 (1.96 g, 9.21 mmol) in DCM (20 mL) was added DMP (4.1 g, 1.05 eq.). The reaction was stirred at room temperature for 4 h and filtered through a glass sinter. The solution was diluted with ethyl acetate and washed with sat. NaHCO₃. The combined organic layers were dried over sodium sulfate and concentrated in vacuo. The crude product was purified by FCC (35% EA:PE) to give the product 2 as an oil (1.05 g, 54%).

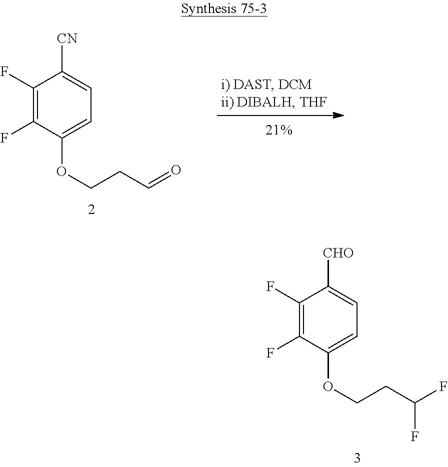 [SMILES: N#Cc1ccc(OCCC=O)c(F)c1F ; O=Cc1ccc(OCCC(F)F)c(F)c1F]

To a solution of 2,3-difluoro-4-(3-oxopropoxy)benzonitrile 2 (1.05 g, 4.97 mmol) in DCM (10 mL) was added DAST (3.2 g, 4 eq.) and the reaction stirred for 6 h. The reaction was carefully quenched with sat. NaHCO₃ and extracted with DCM. The combined organic layers were concentrated in vacuo and redissolved in THF (10 mL). The reaction was cooled to −10° C. and DiBAlH (1 eq.) was added dropwise. The reaction was warmed to room temperature and stirred for 2 h, then quenched with sat. NaHCO₃. The reaction was extracted with ethyl acetate and the combined organic layers dried over sodium sulfate and concentrated in vacuo. The crude product was purified by silica gel chromatography to give the product 3 as an off white solid (250 mg, 21%).

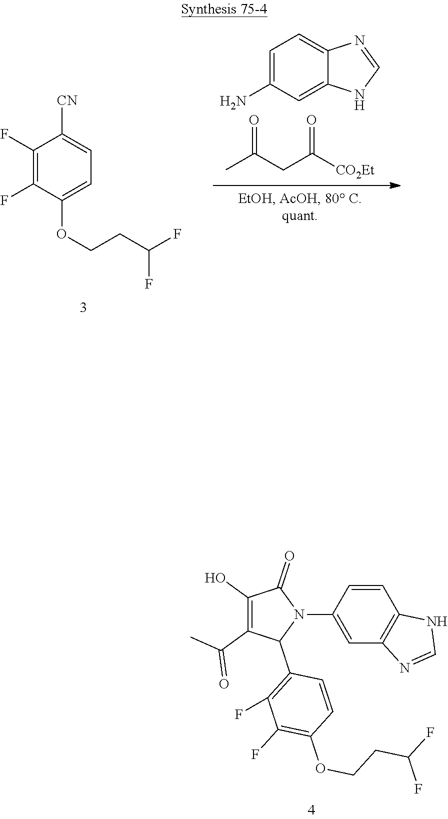

A solution of 4-(3,3-difluoropropoxy)-2,3-difluorobenzaldehyde (250 mg, 1.06 mmol) and 1H-benzo[d]imidazol-6-amine (141 mg, 1.0 eq.) was heated in ethanol (3 mL) and acetic acid (0.3 mL) for 20 mins. Next, ethyl 2,4-dioxopentanoate (167 mg, 1.0 eq.) was added dropwise and the reaction was heated for a further 2 h. The reaction was cooled to room temperature and diluted with ethyl acetate. The solid was filtered to give the desired product as a light green powder (507 mg).

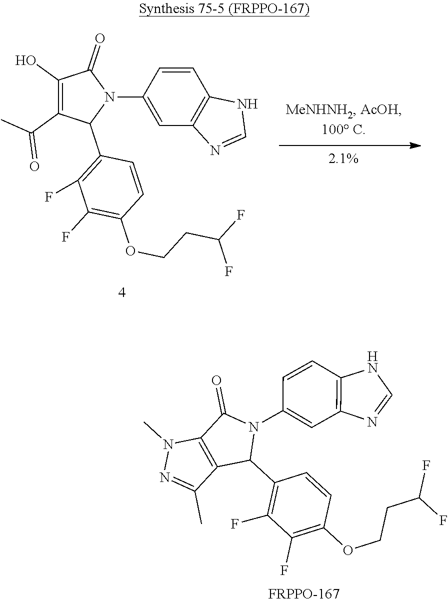 [SMILES: CC(=O)C1=C(O)C(=O)N(c2ccc3[nH]cnc3c2)C1c1ccc(OCCC(F)F)c(F)c1F ; Cc1nn(C)c2c1C(c1ccc(OCCC(F)F)c(F)c1F)N(c1ccc3[nH]cnc3c1)C2=O]

To a solution of 4-acetyl-1-(1H-benzo[d]imidazol-6-yl)-5-(4-(3,3-difluoropropoxy)-2,3-difluorophenyl)-3-hydroxypyrrolidin-2-one 4 (300 mg) in acetic acid (2 mL) was added methyl hydrazine (100 μL) dropwise. The reaction was heated to 100° C. for 6 h. The reaction was quenched with ice and extracted with ethyl acetate. The combined organic layers was washed with sat. NaHCO₃, dried over sodium sulfate and concentrated in vacuo. The product was purified by silica gel chromatography followed by preparative reverse phase LC and finally recrystallization in Et₂O to give FRPPO-167 as a crystalline white solid (6.2 mg, 2.1%, 96.35% purity).

¹H NMR (400 MHz, Acetonitrile-d₃) δ 8.01 (s, 1H), 7.59 (d, J=2.0 Hz, 1H), 7.49 (d, J=8.7 Hz, 1H), 6.84 (td, J=8.4, 2.3 Hz, 1H), 6.68 (ddd, J=9.2, 7.4, 1.9 Hz, 1H), 6.24 (s, 1H), 5.97 (tt, J=56.4, 4.6 Hz, 1H), 4.04 (t, J=6.1 Hz, 2H), 3.90 (s, 3H), 2.20 (ttd, J=17.0, 6.1, 4.6 Hz, 2H), 1.95 (s, 3H).

LC: Gradient 5% to 95% MeCN:H₂O (0.1% HCOOH), KromegaSil OSD-H, 6.03 min.

LRMS: C₂₃H₂₀F₄N₅O₂ requires 474.150. Found 474.161.

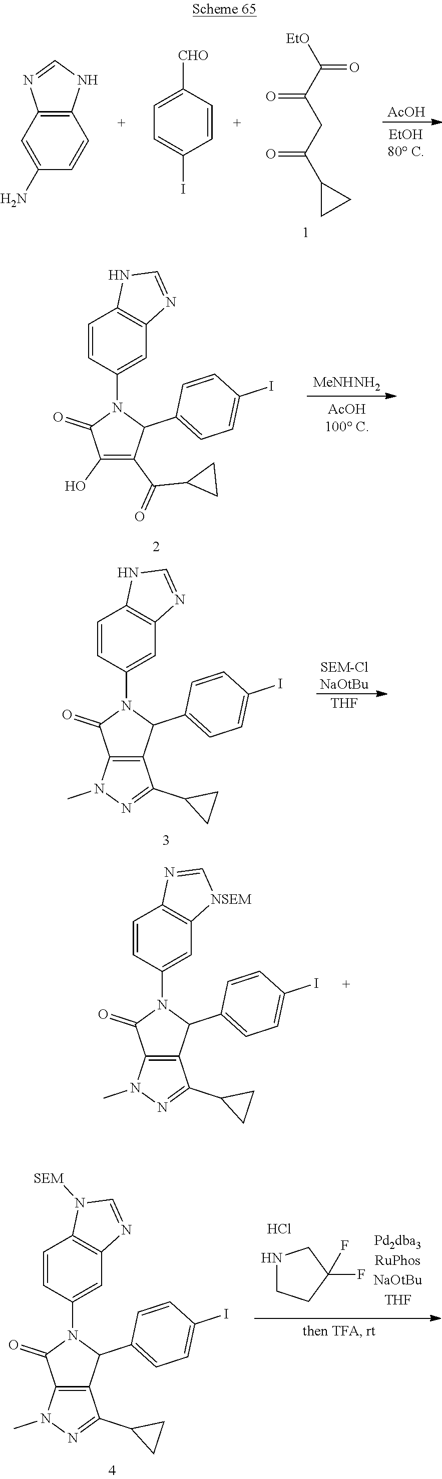

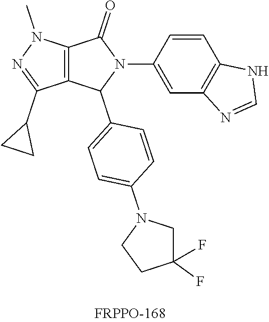 [SMILES: Cn1nc(C2CC2)c2c1C(=O)N(c1ccc3[nH]cnc3c1)C2c1ccc(N2CCC(F)(F)C2)cc1]

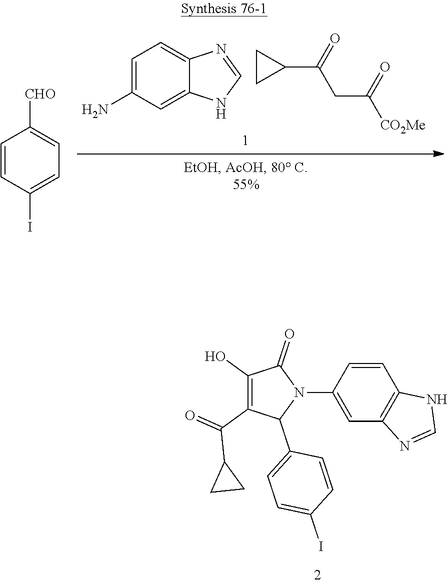

A solution of 4-iodobenzaldehyde (1.36 mg, 5.88 mmol) and 1H-benzo[d]imidazol-6-amine (782 mg, 1.0 eq.) in ethanol (10 mL) and acetic acid (0.5 mL) was heated at 80° C. for 20 mins. Methyl 4-cyclopropyl-2,4-dioxobutanoate 1 (1.0 g, 1.0 eq.) was added dropwise and the reaction was heated overnight. The reaction was cooled to 0° C. and compound 2 was collected by filtration and isolated as a brown solid (1.57 g, 55%).

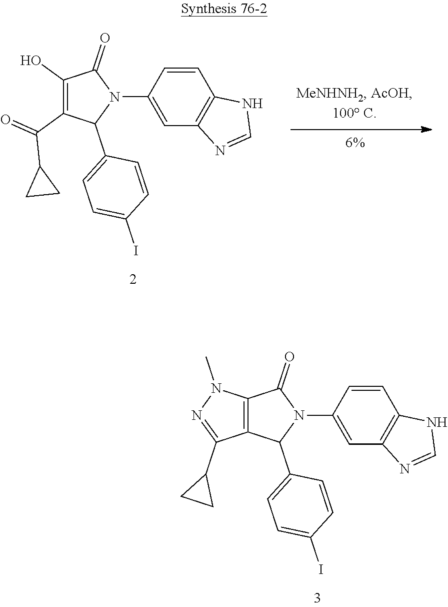 [SMILES: O=C(C1=C(O)C(=O)N(c2ccc3[nH]cnc3c2)C1c1ccc(I)cc1)C1CC1 ; Cn1nc(C2CC2)c2c1C(=O)N(c1ccc3[nH]cnc3c1)C2c1ccc(I)cc1]

To a solution of 2 in acetic acid (10 mL) was added methylhydrazine (352 μL, 2.0 eq.) dropwise. The reaction was heated to 100° C. overnight. The reaction was quenched with sat. Na₂CO₃ and extracted with DCM. The combined organic layers were dried over sodium sulfate and concentrated in vacuo. The crude product was purified by FCC to give 3 as a tan solid (105 mg, 6%). The desired regioisomer 3 elutes first by FCC in 5% MeOH/DCM.

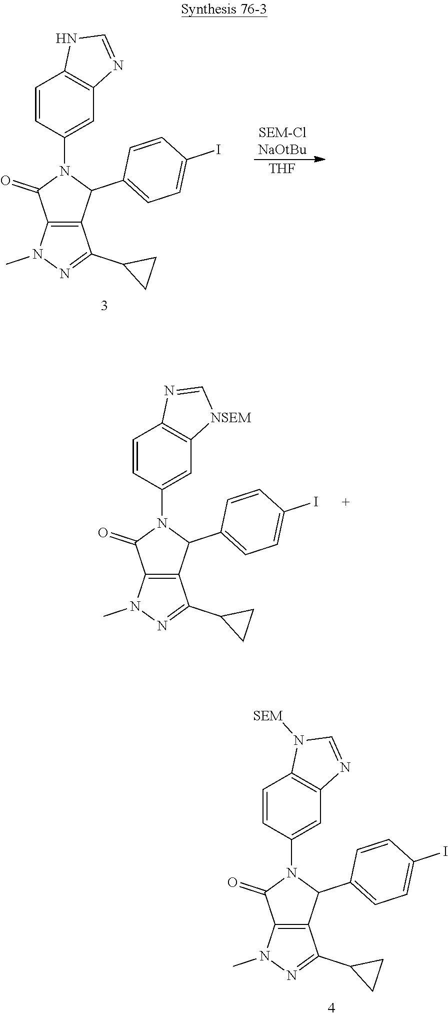 [SMILES: Cn1nc(C2CC2)c2c1C(=O)N(c1ccc3[nH]cnc3c1)C2c1ccc(I)cc1 ; "SEM-ClNaOtBu" ; "THF" ; Cn1nc(C2CC2)c2c1C(=O)N(c1ccc3ncn(COCC[Si](C)(C)C)c3c1)C2c1ccc(I)cc1 ; Cn1nc(C2CC2)c2c1C(=O)N(c1ccc3c(c1)ncn3COCC[Si](C)(C)C)C2c1ccc(I)cc1]

To a solution of 3 (100 mg, 0.206 mmol) in THF (5 mL) was added NaOtBu (24 mg, 1.2 eq.) and SEMCl (44 μL, 1.2 eq.) The reaction was stirred at rt for 1 h and quenched with sat. NaHCO₃. The reaction was extracted with DCM and the combined organic layers dried over sodium sulfate and concentrated in vacuo. The crude product was purified by FCC to give the mixture of SEM isomers 4 as an off white solid (98 mg, 76%).

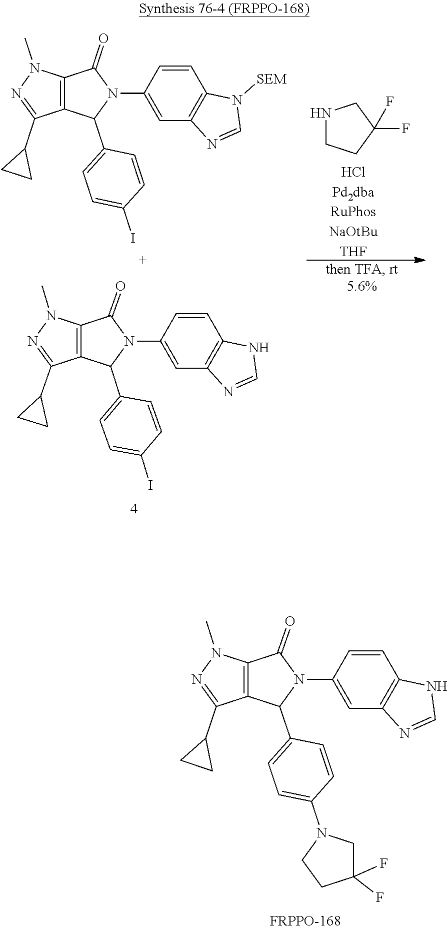 [SMILES: Cn1nc(C2CC2)c2c1C(=O)N(c1ccc3c(c1)ncn3COCC[Si](C)(C)C)C2c1ccc(I)cc1 ; FC1(F)CCNC1 ; "HClPd2dbaRuPhosNaOtBuTHF" ; "then TFA, rt5.6%" ; Cn1nc(C2CC2)c2c1C(=O)N(c1ccc3[nH]cnc3c1)C2c1ccc(I)cc1 ; Cn1nc(C2CC2)c2c1C(=O)N(c1ccc3[nH]cnc3c1)C2c1ccc(N2CCC(F)(F)C2)cc1]

A solution of 4 (40 mg, 0.064 mmol), 3,3-difluoropyrrolidine hydrochloride (18.2 mg, 2.0 eq.), Pd₂dba₃ (17.6 mg, 0.3 eq.), RuPhos (17.9 mg, 0.6 eq.) and sodium tertbutoxide (24.6 mg, 4 eq.) in THF (2 mL) was heated to 65° C. for 2 h, and monitored by LCMS. The reaction was quenched with sat. NaHCO₃ and extracted with DCM. The combined organic layers was dried over sodium sulfate and concentrated in vacuo. The residue was dissolved in TFA (1 mL) and stirred at rt for 6 h. The reaction was quenched with sat. NaHCO₃ and extracted with DCM. The organic layers were dried over sodium sulfate and concentrated in vacuo. The crude product was purified by silica gel chromatography followed by reverse phase LC to give FRPPO-168 as a tan solid (1.7 mg, 5.6%, 96.96% purity).

¹H NMR (400 MHz, Acetonitrile-d₃) δ 7.97 (s, 1H), 7.55 (s, 1H), 7.49-7.10 (m, 2H), 7.10-6.99 (m, 2H), 6.48-6.33 (m, 2H), 5.96 (d, J=7.8 Hz, 1H), 3.96 (s, 3H), 3.56 (t, J=13.3 Hz, 2H), 3.40 (t, J=7.2 Hz, 2H), 2.42 (tt, J=14.4, 7.2 Hz, 2H), 1.74 (ddt, J=15.8, 10.2, 3.8 Hz, 1H), 1.01-0.50 (m, 4H).

LC: Gradient 5% to 95% MeCN:H₂O (0.1% HCOOH), KromegaSil OSD-H, 6.30 min.

LRMS: C₂₅H₂₅F₂N₆O requires 475.206. Found 475.241.

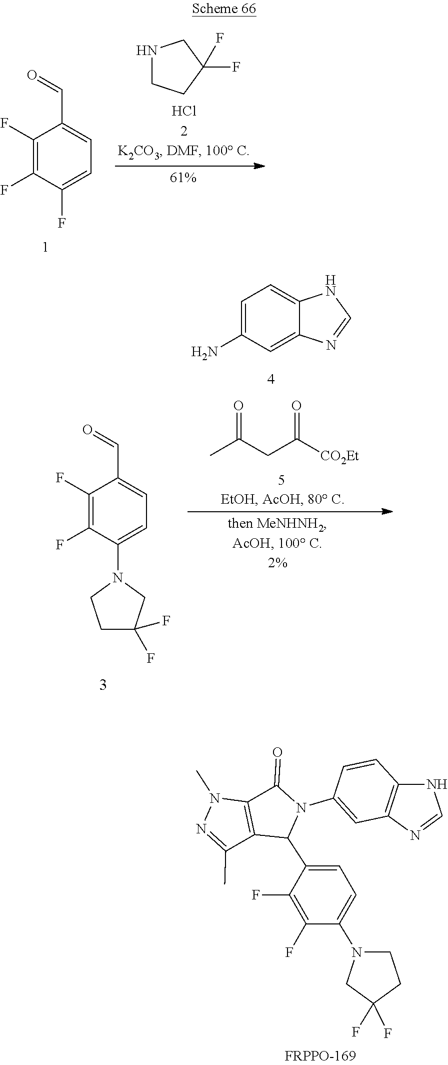 [SMILES: O=Cc1ccc(F)c(F)c1F ; FC1(F)CCNC1 ; Nc1ccc2[nH]cnc2c1 ; O=Cc1ccc(N2CCC(F)(F)C2)c(F)c1F ; CCOC(=O)C(=O)CC(C)=O ; Cc1nn(C)c2c1C(c1ccc(N3CCC(F)(F)C3)c(F)c1F)N(c1ccc3[nH]cnc3c1)C2=O]

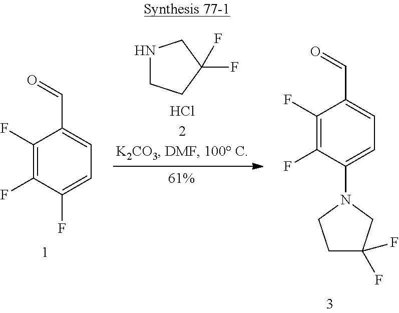 [SMILES: O=Cc1ccc(F)c(F)c1F ; FC1(F)CCNC1 ; O=Cc1ccc(N2CCC(F)(F)C2)c(F)c1F]

To a solution of 2,3,4-trifluorobenzaldehyde 1 (228 μL, 2 mmol) in DMF (2 mL) was added 3,3-difluoropyrrolidine hydrochloride 2 (143 mg, 1.0 eq.) and potassium carbonate (414 mg, 3 eq.). The reaction was heated to 100° C. overnight. The reaction was diluted with water and extracted with ethyl acetate. The combined organic layers were washed with water, dried over sodium sulfate and concentrated in vacuo. The crude product was purified by FCC to give 3 as an off-white solid (150 mg, 61%).

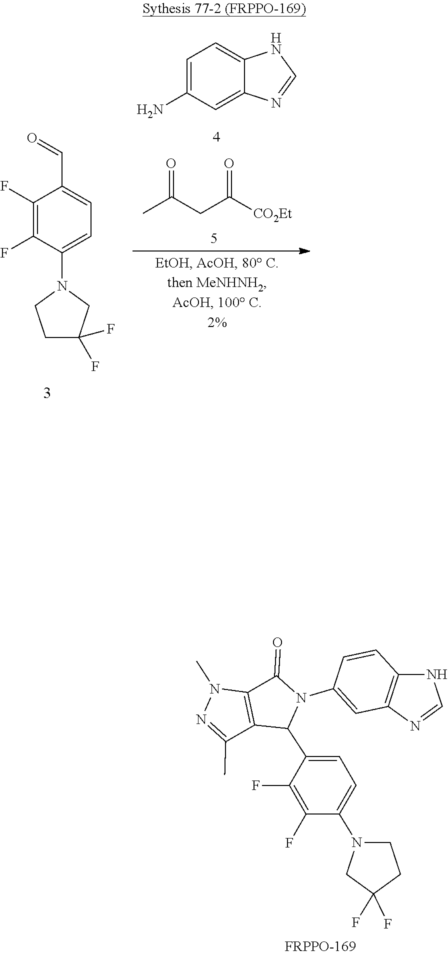 [SMILES: Nc1ccc2[nH]cnc2c1 ; O=Cc1ccc(N2CCC(F)(F)C2)c(F)c1F ; CCOC(=O)C(=O)CC(C)=O ; Cc1nn(C)c2c1C(c1ccc(N3CCC(F)(F)C3)c(F)c1F)N(c1ccc3[nH]cnc3c1)C2=O]

A solution of 3 (150 mg, 0.6068 mmol) and 1H-benzo[d]imidazol-6-amine 4 (81 mg, 1.0 eq.) in ethanol (1 mL) and AcOH (0.05 mL) was heated at 60° C. for 30 mins. ethyl 2,4-dioxopentanoate 5 (85 μL, 1.0 eq.) was added and the reaction was heated for a further 3 h. The reaction was cooled to rt and filtered to give a crude green solid. The green solid was dissolved in AcOH (2 mL) and methylhydrazine (50 μL) was added. The reaction was heated at 100° C. for 4 h. The reaction was quenched with NaHCO₃ and extracted with DCM. The combined organic layers were dried over sodium sulfate and concentrated in vacuo. The crude product was purified by silica gel chromatography followed by reverse phase LC and trituration in Et₂O to give FRPPO-169 as an off-white solid (6.0 mg, 2%, 97.17% purity).

¹H NMR (400 MHz, DMSO-d₆) δ 12.47 (s, 1H), 8.19 (d, J=3.8 Hz, 1H), 7.65 (d, J=17.8 Hz, 1H), 7.52 (dd, J=45.0, 8.6 Hz, 1H), 7.28 (dd, J=27.1, 8.6 Hz, 1H), 6.91 (d, J=8.2 Hz, 1H), 6.65-6.35 (m, 2H), 3.95 (s, 3H), 3.71 (t, J=13.2 Hz, 2H), 3.48 (t, J=7.3 Hz, 2H), 2.40 (dq, J=14.4, 7.2 Hz, 1H), 1.99 (s, 3H).

LC: Gradient 5% to 95% MeCN:H₂O (0.1% HCOOH), KromegaSil OSD-H, 7.88 min.

LRMS: C₂₄H₂₁F₄N₆O requires 485.171. Found 485.009.

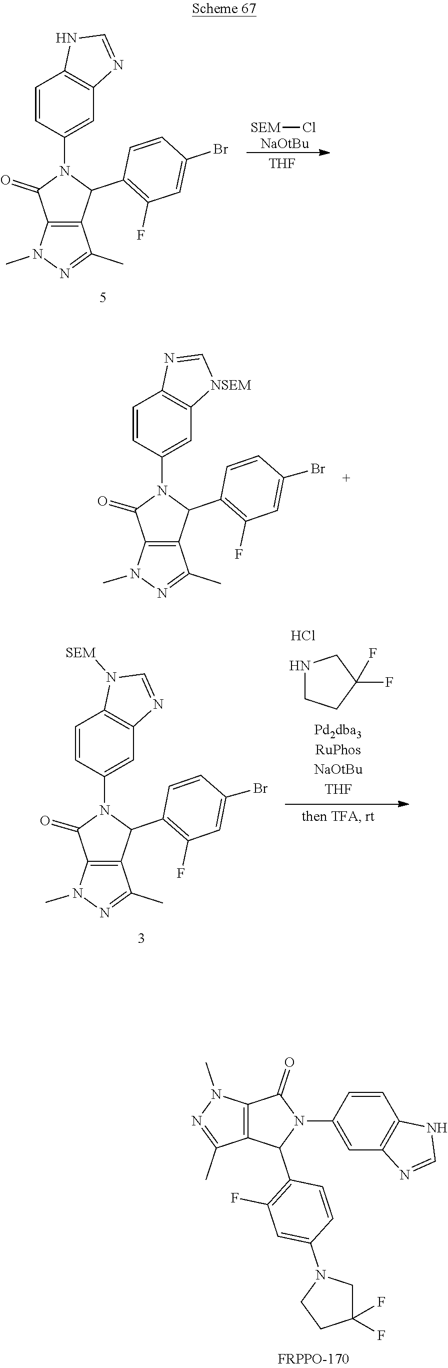

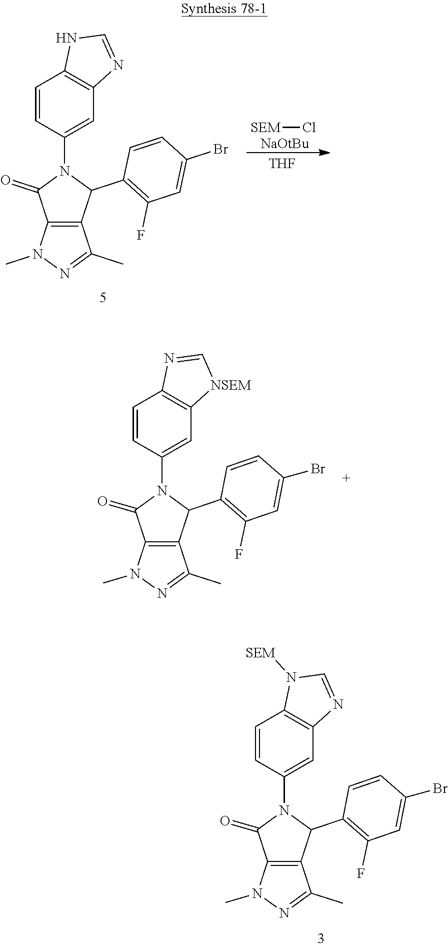 [SMILES: Cc1nn(C)c2c1C(c1ccc(Br)cc1F)N(c1ccc3[nH]cnc3c1)C2=O ; C[Si](C)(C)CCOCCl ; Cc1nn(C)c2c1C(c1ccc(Br)cc1F)N(c1ccc3ncn(COCC[Si](C)(C)C)c3c1)C2=O ; "NaOtBu" ; Cc1nn(C)c2c1C(c1ccc(Br)cc1F)N(c1ccc3c(c1)ncn3COCC[Si](C)(C)C)C2=O ; "THF"]

To a solution of compound 5 (from Scheme 62 and Synthesis 72-1) (550 mg, 1.25 mmol) in THF (10 mL) was added NaOtBu (156 mg, 1.3 eq.) followed by SEMCl (287 μL, 1.3 eq.). The reaction was stirred at rt for 2 h, quenched with sat. NaHCO₃ and extracted with DCM. The organic layers were dried over sodium sulfate and concentrated in vacuo. The crude product was purified by FCC to give 3 as a tan solid (472 mg, 66%). The product was isolated as mixture of N-SEM regioisomers.

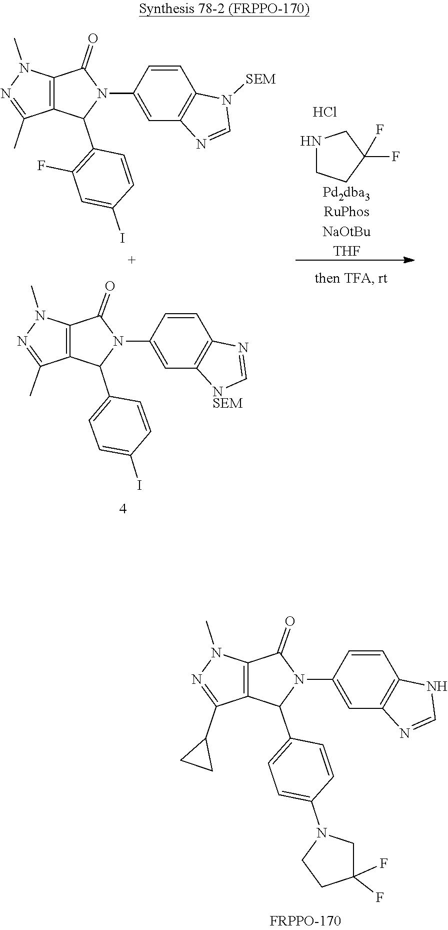

A solution of 3 (100 mg, 0.176 mmol), 3,3-difluoropyrollidine hydrochloride (50 mg, 2.0 eq.), Pd₂dba₃ (40.3 mg, 0.25 eq.), RuPhos (41 mg, 0.5 eq. and sodium tert-butoxide (50.8 mg, 3.0 eq.) in THF (5 mL) was heated at 60° C. overnight. The reaction was diluted with sat. NaHCO₃ and extracted with DCM. The combined organic layers were dried over sodium sulfate and concentrated in vacuo. The residue was dissolved in TFA (1 mL) and stirred at rt for 6 h. The reaction was quenched with sat. NaHCO₃ and extracted with DCM. The organic layers were washed with sodium sulfate and concentrated in vacuo. The crude product was purified by silica gel chromatography followed by reverse phase LC (neutral condition) followed by trituration in Et₂O/hexanes to give FRPPO-170 as an off white solid (4.1 mg, 5%, 98.45% purity).

¹H NMR (400 MHz, DMSO-d₆) δ 8.17 (s, 1H), 7.62 (d, J=1.9 Hz, 1H), 7.49 (d, J=8.6 Hz, 1H), 7.26 (dd, J=8.6, 2.0 Hz, 1H), 7.02 (t, J=8.8 Hz, 1H), 6.41 (s, 1H), 6.33-6.23 (m, 2H), 3.95 (s, 3H), 3.59 (t, J=13.2 Hz, 2H), 3.44-3.34 (m, 2H), 2.42 (dt, J=14.4, 7.2 Hz, 2H), 1.97 (s, 3H).

LC: Gradient 5% to 95% MeCN:H₂O (0.1% HCOOH), KromegaSil OSD-H, 7.59 min.

LRMS: C₂₄H₂₂F₃N₆O requires 467.181. Found 467.003.

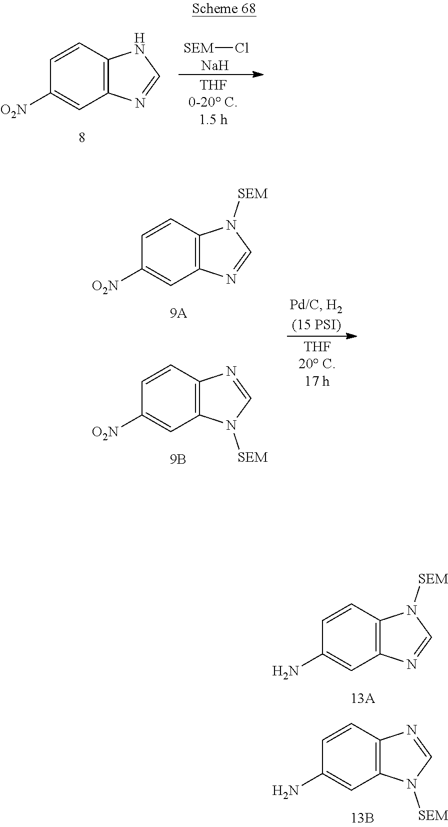

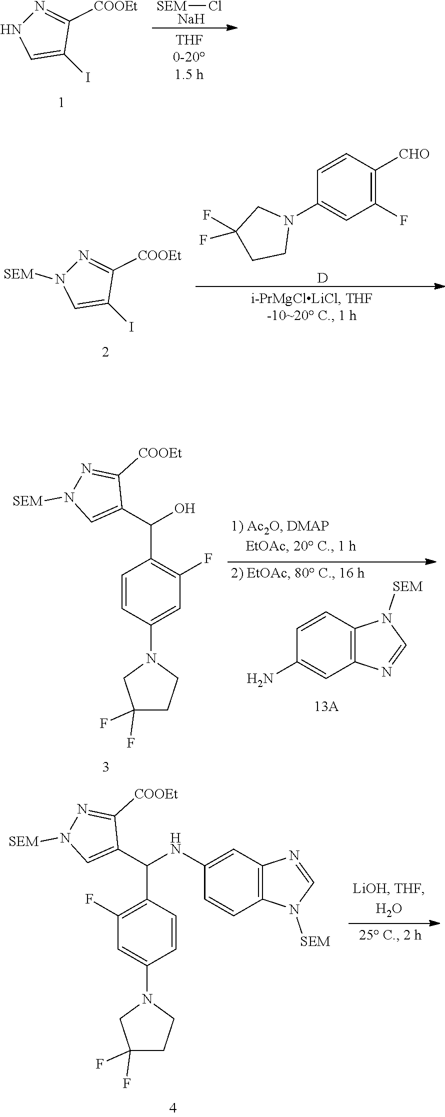

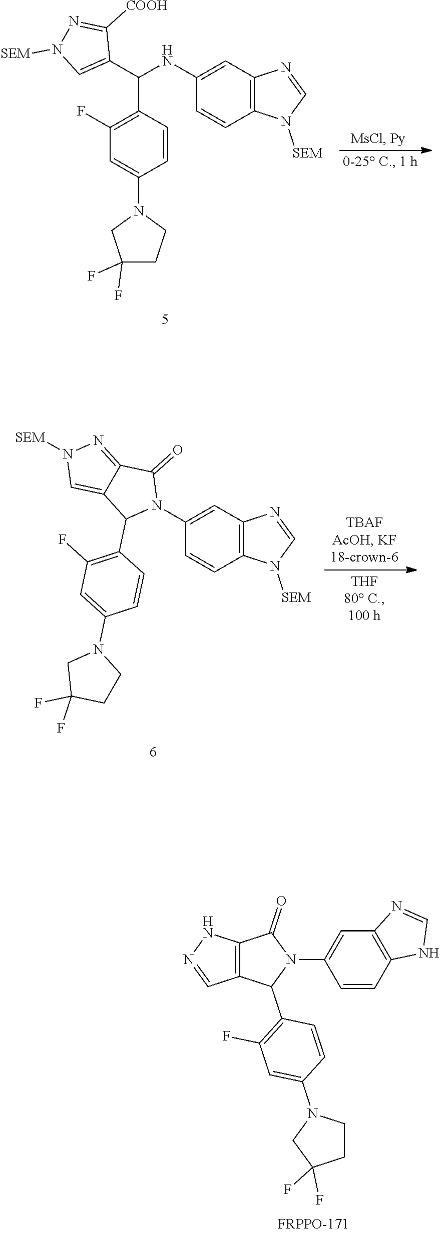 [SMILES: C[Si](C)(C)CCOCn1cc(C(Nc2ccc3c(c2)ncn3COCC[Si](C)(C)C)c2ccc(N3CCC(F)(F)C3)cc2F)c(C(=O)O)n1 ; C[Si](C)(C)CCOCn1cc2c(n1)C(=O)N(c1ccc3c(c1)ncn3COCC[Si](C)(C)C)C2c1ccc(N2CCC(F)(F)C2)cc1F ; O=C1c2[nH]ncc2C(c2ccc(N3CCC(F)(F)C3)cc2F)N1c1ccc2[nH]cnc2c1]

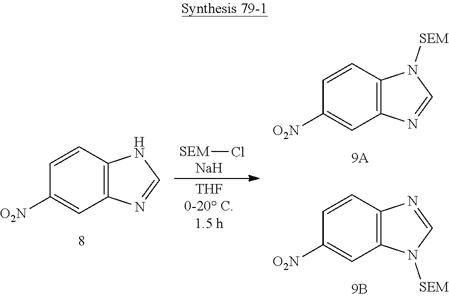

To a mixture of 5-nitro-1H-benzimidazole (4 g, 24.52 mmol, 1 eq) in THF (50 mL) was added NaH (1.47 g, 36.78 mmol, 60% purity, 1.5 eq) at 0° C. under N₂. The mixture was stirred at 0° C. for 30 min, then SEM-Cl (4.91 g, 29.42 mmol, 5.21 mL, 1.2 eq) was added at 0° C. and stirred at 20° C. for 1 hr. The mixture was poured into saturated NH₄Cl (50 mL). The aqueous phase was extracted with ethyl acetate (100 mL*3). The combined organic phase was washed with brine (100 mL), dried with anhydrous Na₂SO₄, filtered and concentrated in vacuum. The residue was purified by silica gel chromatography (column height: 250 mm, diameter: 100 mm, 100-200 mesh silica gel, petroleum ether:ethyl acetate=1:1, 0:1). A mixture of compound 9A and 9B (5.1 g, 17.38 mmol, 70.89% yield) were obtained as yellow oil.

¹H NMR (400 MHz, CDCl₃) δ=8.79-8.74 (m, 1H), 8.53 (d, J=2.1 Hz, 1H), 8.32-8.24 (m, 1H), 8.22 (s, 1H), 8.16 (s, 1H), 7.90 (d, J=9.0 Hz, 1H), 7.63 (d, J=8.9 Hz, 1H), 5.62 (s, 1H), 5.60 (s, 1H), 3.61-3.50 (m, 2H), 1.00-0.89 (m, 2H), −0.02-−0.05 (m, 9H).

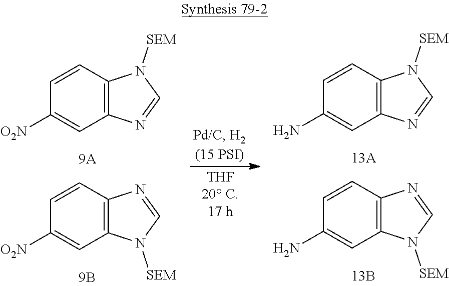 [SMILES: C[Si](C)(C)CCOCn1cnc2cc([N+](=O)[O-])ccc21 ; C[Si](C)(C)CCOCn1cnc2cc(N)ccc21 ; "Pd/C, H2(15 PSI)" ; C[Si](C)(C)CCOCn1cnc2ccc([N+](=O)[O-])cc21 ; C[Si](C)(C)CCOCn1cnc2ccc(N)cc21]

To a solution of compound 9 (5.1 g, 17.38 mmol, 1 eq) in THF (50 mL) was added Pd/C (0.5 g, 10% purity) under N₂. The suspension was degassed under vacuum and purged with H₂ several times. The mixture was stirred under H₂ (15 psi) at 20° C. for 17 hr. The reaction mixture was filtered and the filter was concentrated. A mixture of compound 13A and 13B (ratio 5:4) (4.1 g, crude) were obtained as yellow oil.

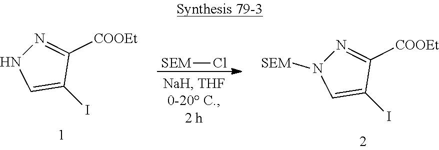 [SMILES: CCOC(=O)c1n[nH]cc1I ; C[Si](C)(C)CCOCCl ; CCOC(=O)c1nn(COCC[Si](C)(C)C)cc1I]

To a mixture of ethyl 4-iodo-1H-pyrazole-3-carboxylate 1 (5 g, 18.79 mmol, 1 eq) in THF (50 mL) was added NaH (902.05 mg, 22.55 mmol, 60% purity, 1.2 eq) at 0° C. under N₂. The mixture was stirred at 0° C. for 30 min, then SEM-Cl (4.70 g, 28.19 mmol, 4.99 mL, 1.5 eq) was added at 0° C. and stirred at 20° C. for 1 hr. TLC (petroleum ether:ethyl acetate=2:1) showed the ethyl 4-iodo-1H-pyrazole-3-carboxylate was consumed and new spots were detected. The mixture was poured into saturated NH₄Cl (50 mL) aqueous. The aqueous phase was extracted with ethyl acetate (80 mL*3). The combined organic phase was washed with brine (100 mL), dried with anhydrous Na₂SO₄, filtered and concentrated in vacuum. The residue was purified by silica gel chromatography (100-200 mesh silica gel, petroleum ether:ethyl acetate=5:1, 2:1). Compound 2 (5.1 g, 12.87 mmol, 68.47% yield) was obtained as colorless oil.

¹H NMR (400 MHz, CDCl₃) δ=7.74 (d, J=0.9 Hz, 1H), 5.49 (d, J=0.8 Hz, 2H), 4.51-4.39 (m, 2H), 3.58 (dt, J=0.8, 8.3 Hz, 2H), 1.44 (dt, J=0.9, 7.1 Hz, 3H), 1.00-0.86 (m, 2H), -0.01 (d, J=1.0 Hz, 9H).

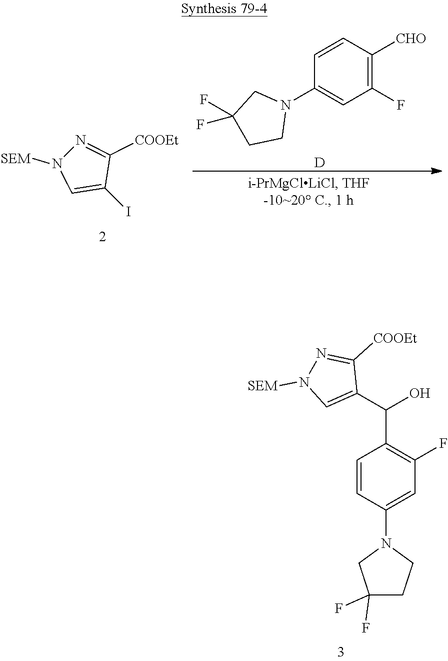

To a mixture of compound 2 (2 g, 5.05 mmol, 1 eq) in THF (20 mL) was added i-PrMgCl—LiCl (1.3 M, 4.66 mL, 1.2 eq) at −10° C. under N₂. The mixture was stirred at −10° C. for 10 min, compound D (from Scheme 40 and Synthesis 44-1) (1.36 g, 5.05 mmol, 85% purity, 1 eq) was added at −10° C. and stirred for 20° C. for 1 hr. TLC (petroleum ether:ethyl acetate=2:1) showed the ethyl compound 2 was consumed and the new spots were detected. The mixture was poured into saturated NH₄Cl (50 mL) aqueous and stirred for 5 min. The aqueous phase was extracted with ethyl acetate (50 mL*3). The combined organic phase was washed with brine (50 mL), dried with anhydrous Na₂SO₄, filtered and concentrated in vacuum. The residue was purified by column chromatography (SiO₂, petroleum ether:ethyl acetate=10:1 to 2:1). Compound 3 (1.5 g, 3.00 mmol, 59.49% yield) was obtained as yellow oil.

¹H NMR (400 MHz, CDCl₃) δ=7.43 (t, J=8.5 Hz, 1H), 7.18 (s, 1H), 6.36 (dd, J=2.2, 8.4 Hz, 1H), 6.27-6.19 (m, 2H), 5.40 (s, 2H), 4.77 (d, J=4.6 Hz, 1H), 4.48 (ttd, J=3.5, 7.1, 10.6 Hz, 2H), 3.67 (t, J=13.1 Hz, 2H), 3.58-3.49 (m, 4H), 2.51 (tt, J=7.1, 13.9 Hz, 2H), 1.43 (t, J=7.1 Hz, 3H), 0.91-0.86 (m, 3H), −0.02 (s, 9H).

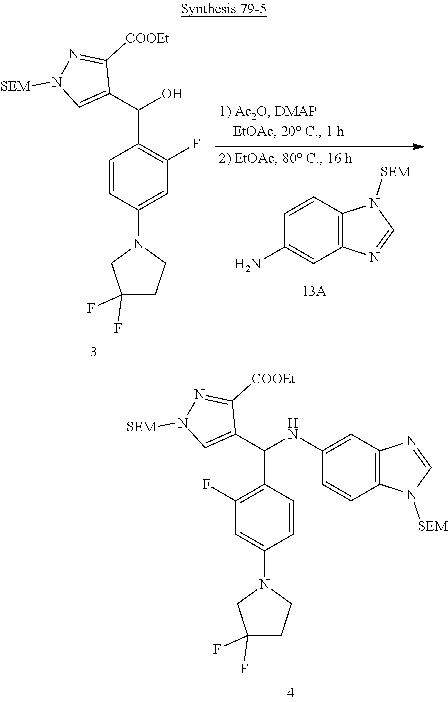 [SMILES: CCOC(=O)c1nn(COCC[Si](C)(C)C)cc1C(O)c1ccc(N2CCC(F)(F)C2)cc1F ; C[Si](C)(C)CCOCn1cnc2cc(N)ccc21 ; CCOC(=O)c1nn(COCC[Si](C)(C)C)cc1C(Nc1ccc2c(c1)ncn2COCC[Si](C)(C)C)c1ccc(N2CCC(F)(F)C2)cc1F]

To a mixture of compound mg, 640.52 μmol, eq and DMAP (234.76 mg, 1.92 mmol, 3 eq) in EtOAc (5 mL) was added Ac₂O (85.01 mg, 832.67 μmol, 77.99 μL, 1.3 eq) at 20° C. under N₂. The mixture was stirred at 20° C. for 1 hr. Then compound 13A was added, the reaction mixture was stirred at 80° C. for 16 hrs. The reaction mixture was monitored by TLC (ethyl acetate:ethanol=30:1), compound 13A was remained, one main spot was observed. The reaction mixture was concentrated in vacuum and the residue was purified by silica gel chromatography (petroleum ether:ethyl acetate=5:1, 0:1). Compound 4 (260 mg, 335.04 μmol, 52.31% yield, 96% purity) was obtained as yellowish oil.

LCMS: RT=1.615 min, m/z=745.4 (M+1)⁺.

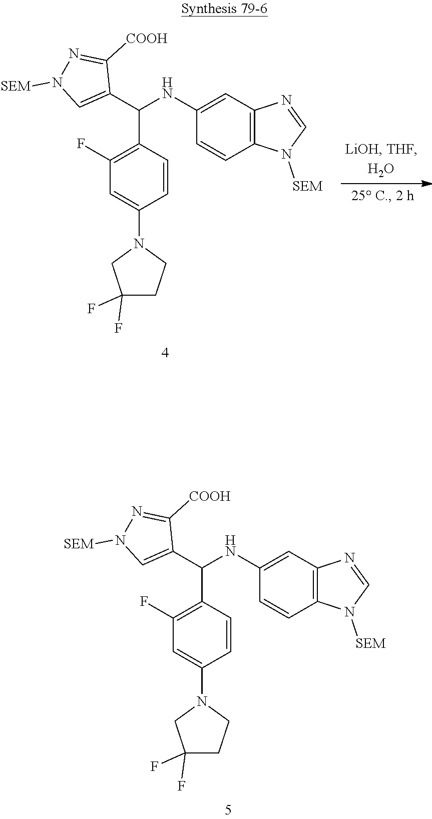 [SMILES: C[Si](C)(C)CCOCn1cc(C(Nc2ccc3c(c2)ncn3COCC[Si](C)(C)C)c2ccc(N3CCC(F)(F)C3)cc2F)c(C(=O)O)n1 ; C[Si](C)(C)CCOCn1cc(C(Nc2ccc3c(c2)ncn3COCC[Si](C)(C)C)c2ccc(N3CCC(F)(F)C3)cc2F)c(C(=O)O)n1]

To a solution of compound 4 (260 mg, 349.00 μmol, 1 eq) in THF (4 mL), MeOH (2 mL) and H₂O (1 mL) was added LiOH (41.8 mg, 1.745 mmol, 5 eq). The mixture was stirred at 25° C. for 2 hrs. TLC (Dichloromethane:Methanol=10:1; R_{f}=0.31) showed there was no compound 4 remained and one main new spot was detected. The mixture was adjusted pH to about 7 with 1 M HCl. The resulting mixture was extracted with EtOAc (5 mL*3). The combined organic phase was dried over Na₂SO₄, filtered and concentrated at reduced pressure to give compound 5 (220 mg, 306.86 μmol, 87.93% yield) as yellow solid.

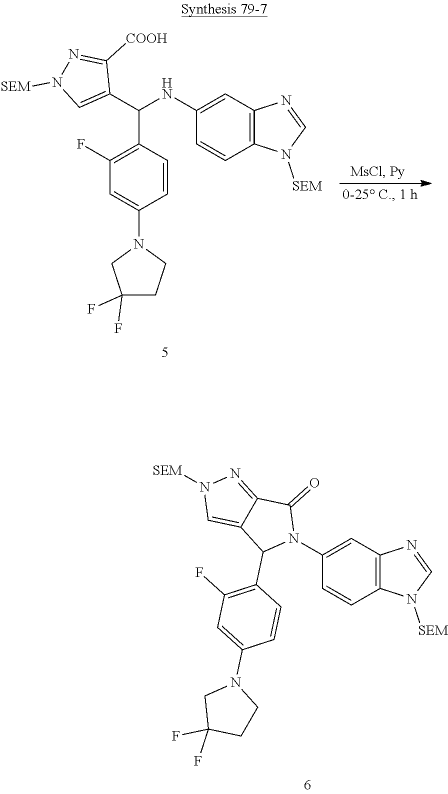 [SMILES: C[Si](C)(C)CCOCn1cc(C(Nc2ccc3c(c2)ncn3COCC[Si](C)(C)C)c2ccc(N3CCC(F)(F)C3)cc2F)c(C(=O)O)n1 ; C[Si](C)(C)CCOCn1cc2c(n1)C(=O)N(c1ccc3c(c1)ncn3COCC[Si](C)(C)C)C2c1ccc(N2CCC(F)(F)C2)cc1F]

To a solution of compound 5 (220 mg, 306.86 μmol, 1 eq) in DCM (5 mL) was added Py (48.55 mg, 613.72 μmol, 49.54 μL, 2 eq) and MsCl (52.73 mg, 460.29 μmol, 35.63 μL, 1.5 eq) at 0° C. The mixture was stirred at 25° C. for 1 hr. TLC (dichloromethane:methanol=10:1; RF=0.72) showed there was no compound 5 remained and one main new spot was detected. The mixture was quenched with H₂O (5 mL) and extracted with DCM (5 mL*3). The organic layers were washed with brine (10 mL*2), dried over Na₂SO₄ and concentrated under vacuum to give a residue. The residue was purified by silica gel chromatography (100-200 mesh silica gel, petroleum ether:ethyl acetate=5:1, 0:1) to give compound 6 (100 mg, 143.08 μmol, 46.63% yield) as yellow oil.

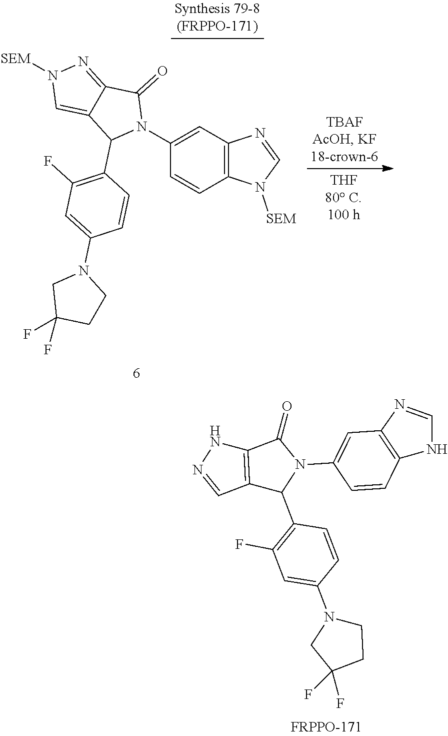 [SMILES: C[Si](C)(C)CCOCn1cc2c(n1)C(=O)N(c1ccc3c(c1)ncn3COCC[Si](C)(C)C)C2c1ccc(N2CCC(F)(F)C2)cc1F ; O=C1c2[nH]ncc2C(c2ccc(N3CCC(F)(F)C3)cc2F)N1c1ccc2[nH]cnc2c1]

To a mixture of compound 6 (30 mg, 42.92 μmol, 1 eq) in THF (1 mL) was added 18-C-6 (56.73 mg, 214.62 μmol, 5 eq), TBAF (1 M in THF, 85.85 μL, 2 eq, which was adjusted pH to 7 with AcOH before use) and KF (12.47 mg, 214.62 μmol, 5.03 μL, 5 eq). The mixture was stirred at 80° C. for 100 hrs. The mixture was diluted with EtOAc (20 mL) and the resulting mixture was washed with water (10 mL*5), brine (10 mL*2), dried over Na₂SO₄, filtered and concentrated at reduced pressure to give a residue. The residue was purified by prep-HPLC (column: Phenomenex Gemini-NX C18 75*30 mm*3 μm; mobile phase: [water (0.05% ammonia hydroxide v/v)-ACN]; B %: 14%-44%, 11.5 min) to give a yellow solid. The solid was purified by prep-HPLC (column: Unisil 3-100 C18 Ultra 150*50 mm*3 μm; mobile phase: [water (0.225% FA)-ACN]; B %: 15%-35%, 10 min) to give FRPPO-171 (1 mg, 2.28 μmol, 5.31% yield, 100% purity) as white solid.

¹H NMR (400 MHz, CD₃CN) δ=8.02 (s, 1H), 7.75-7.57 (m, 2H), 7.56 (s, 1H), 7.35-7.25 (m, 1H), 7.03-6.95 (m, 1H), 6.45-6.36 (m, 1H), 6.26-6.16 (m, 2H), 3.58 (t, J=13.2 Hz, 2H), 3.42 (t, J=7.3 Hz, 2H), 2.51-2.38 (m, 2H).

LCMS: RT=0.682 min, m/z=439.1 (M+1)⁺.

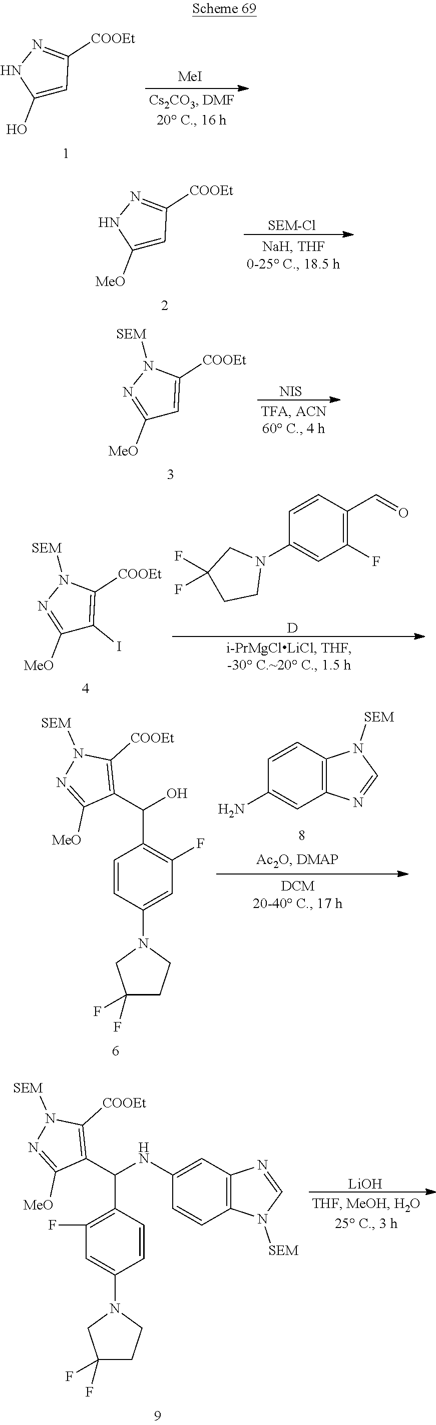

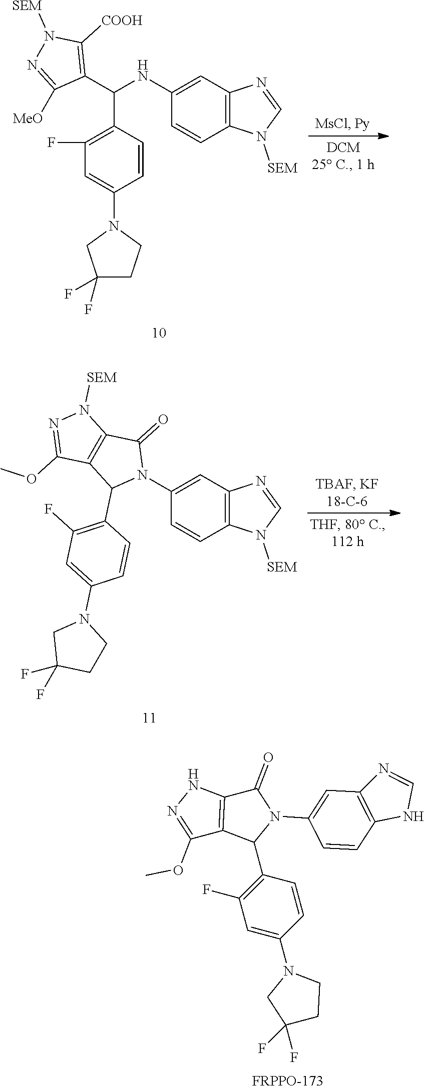 [SMILES: COc1nn(COCC[Si](C)(C)C)c(C(=O)O)c1C(Nc1ccc2c(c1)ncn2COCC[Si](C)(C)C)c1ccc(N2CCC(F)(F)C2)cc1F ; COc1nn(COCC[Si](C)(C)C)c2c1C(c1ccc(N3CCC(F)(F)C3)cc1F)N(c1ccc3c(c1)ncn3COCC[Si](C)(C)C)C2=O ; COc1n[nH]c2c1C(c1ccc(N3CCC(F)(F)C3)cc1F)N(c1ccc3[nH]cnc3c1)C2=O]

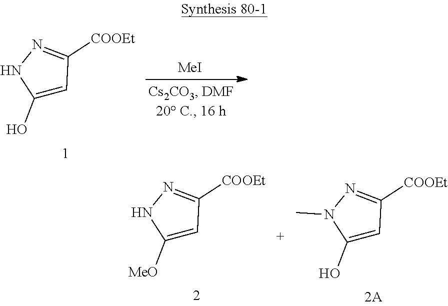

To a mixture of compound 1 (7 g, 44.83 mmol, 1 eq) and Cs₂CO₃ (16.07 g, 49.32 mmol, 1.1 eq) in DMF (70 mL) was added Mel (6.36 g, 44.83 mmol, 2.79 mL, 1 eq). The mixture was stirred at 20° C. for 16 hrs. The mixture was poured into water (200 mL). The aqueous phase was extracted with ethyl acetate (200 mL*3). The combined organic phase was washed with brine (300 mL), dried with anhydrous Na₂SO₄, filtered and concentrated in vacuum. The crude product was purified by reversed-phase HPLC (0.1% FA condition) to give compound 2A (2.5 g, 14.69 mmol, 32.77% yield) and 2.4 g mixture of compound 2 and 2A. The mixture (2.4 g) was purified by prep-HPLC (column: Phenomenex luna C18 250*50 mm*10 μm; mobile phase: [water (0.1% TFA)-ACN]; B %: 5%-35%, 20 min) to give compound 2 (1.7 g, 9.99 mmol, 22.28% yield) as white solid.

### Compound 2:

¹H NMR (400 MHz, DMSO-d₆) δ 6.21 (s, 1H), 4.27 (q, J=7.2 Hz, 2H), 3.79 (s, 3H), 1.28 (t, J=7.2 Hz, 3H).

### Compound 2A:

¹H NMR (400 MHz, DMSO-d₆) δ 10.05 (brs, 1H), 5.98 (s, 1H), 4.26 (q, J=7.2 Hz, 2H), 3.87 (s, 3H), 1.28 (t, J=7.2 Hz, 3H).

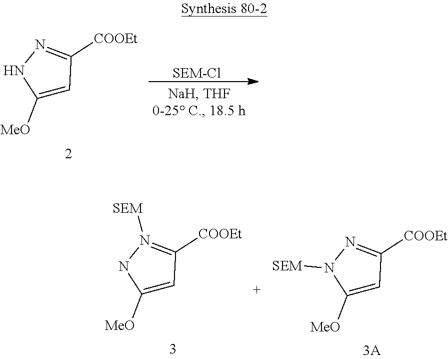

To a solution of compound 2 (1.7 g, 9.99 mmol, 1 eq) in THF (20 mL) was added NaH (479.49 mg, 11.99 mmol, 60% purity, 1.2 eq) at 0° C., the mixture was stirred at 0° C. for 0.25 hr. Then SEM-Cl (2.00 g, 11.99 mmol, 2.12 mL, 1.2 eq) was added, and the mixture was stirred at 25° C. for 18 hrs. The reaction mixture was quenched by saturated NH₄Cl aqueous solution (50 mL), and then diluted with ethyl acetate (100 mL), the organic layers were washed with brine (10 mL*2), dried over Na₂SO₄, filtered and concentrated under reduced pressure to give a residue. The residue was purified by column chromatography on silica gel eluted (petroleum ether:ethyl acetate=20:1 to 3:1) to compound 3A (1.1 g, 3.66 mmol, 36.64% yield) as yellow oil and compound 3 (650 mg, 2.16 mmol, 21.66% yield) as yellow oil.

### Compound 3A:

¹H NMR (400 MHz, CDCl₃) δ=6.09 (s, 1H), 5.39 (s, 2H), 4.41 (q, J=7.1 Hz, 2H), 3.95 (s, 3H), 3.70-3.57 (m, 2H), 1.39 (t, J=7.2 Hz), 0.94-0.88 (m, 2H), 0.02 (s, 9H).

LCMS: RT=0.860 min, m/z=301.1 (M+1)⁺.

### Compound 3:

¹H NMR (400 MHz, CDCl₃) δ=6.30 (s, 1H), 5.70 (s, 2H), 4.35 (q, J=7.1 Hz, 2H), 3.91 (s, 3H), 3.61 (dd, J=7.6, 8.7 Hz, 2H), 1.37 (t, J=7.2 Hz, 3H), 0.95-0.87 (m, 2H), 0.025 (m, 9H).

LCMS: RT=0.911 min, m/z=301.1 (M+1)⁺.

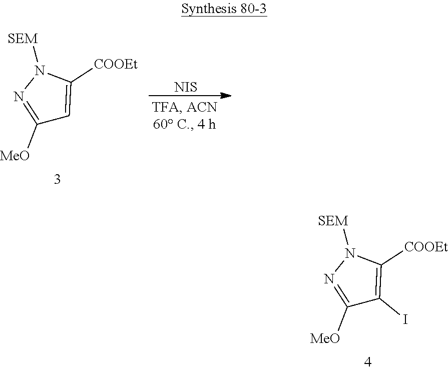 [SMILES: CCOC(=O)c1cc(OC)nn1COCC[Si](C)(C)C ; "NIS" ; CCOC(=O)c1c(I)c(OC)nn1COCC[Si](C)(C)C]

To a solution of compound 3 (0.6 g, 2.00 mmol, 1 eq) in CH₃CN (10 mL) was added NIS (539.20 mg, 2.40 mmol, 1.2 eq). The mixture was stirred at 60° C. for 2 hrs. TLC (petroleum ether:ethyl acetate=10:1) showed compound 3 was remained and new spot was detected. Then TFA (308.00 mg, 2.70 mmol, 0.2 mL, 1.35 eq) was added. The mixture was stirred at 60° C. for 2 hrs. TLC (petroleum ether:ethyl acetate=10:1) showed compound 3 was consumed and new spot was detected. The mixture was quenched with saturated aqueous Na₂SO₃ solution (20 mL). The resulting solution was extracted with EtOAc (50 mL). The organic layers was washed with brine and dried over Na₂SO₄, and concentrated under vacuum to give a residue. The residue was purified by column chromatography on silica gel (petroleum ether:ethyl acetate=20:1 to 5:1) to give compound 4 (750 mg, 1.76 mmol, 88.09% yield) as colorless oil.

¹H NMR (400 MHz, CDCl₃) δ=5.70 (s, 2H), 4.42 (q, J=7.1 Hz, 2H), 3.99 (s, 3H), 3.58 (dd, J=7.7, 8.8 Hz, 2H), 1.44 (t, J=7.2 Hz, 3H), 0.94-0.86 (m, 2H), 0.02 (s, 9H).

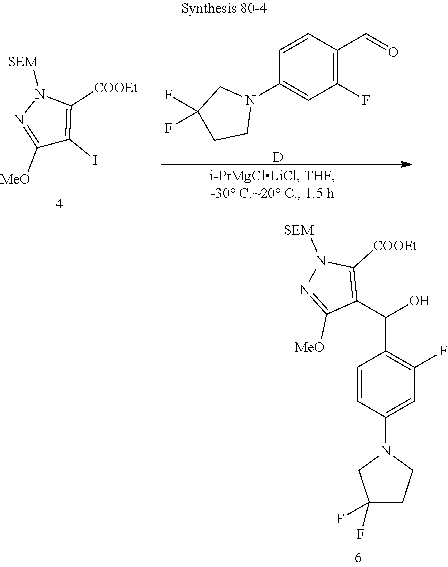

To a mixture of compound 4 (750.00 mg, 1.76 mmol, 1 eq) in THF (10 mL) was added i-PrMgCl—LiCl (1.3 M, 1.62 mL, 1.2 eq) slowly at] −30° C. under N₂. The mixture was stirred at −30° C. for 15 min, then compound D (Scheme 40 and Synthesis 44-1) (403.21 mg, 1.76 mmol, 1 eq) was added and the mixture was stirred at 20° C. for 1 hr. The mixture was quenched with saturated NH₄Cl aqueous solution (20 mL), the resulting solution was extracted with EtOAc (50 mL), the organic layers was washed with brine and dried over Na₂SO₄, and concentrated under vacuum to give a residue. The residue was purified by column chromatography on silica gel eluted (petroleum ether:ethyl acetate=20:1 to 5:1) to give compound 6 (480 mg, 906.30 μmol, 51.52% yield) as colorless oil.

¹H NMR (400 MHz, CDCl₃) δ=7.19 (t, J=8.8 Hz, 1H), 6.33 (d, J=10.3 Hz, 1H), 6.26-6.19 (m, 2H), 5.67 (s, 2H), 4.40-4.27 (m, 2H), 3.81 (d, J=10.4 Hz, 1H), 3.67-3.55 (m, 4H), 3.49 (t, J=7.2 Hz, 2H), 2.48 (tt, J=7.0, 13.9 Hz, 2H), 1.34 (t, J=7.2 Hz, 3H), 0.89 (dd, J=7.8, 8.6 Hz, 2H), 0.024 (m, 9H).

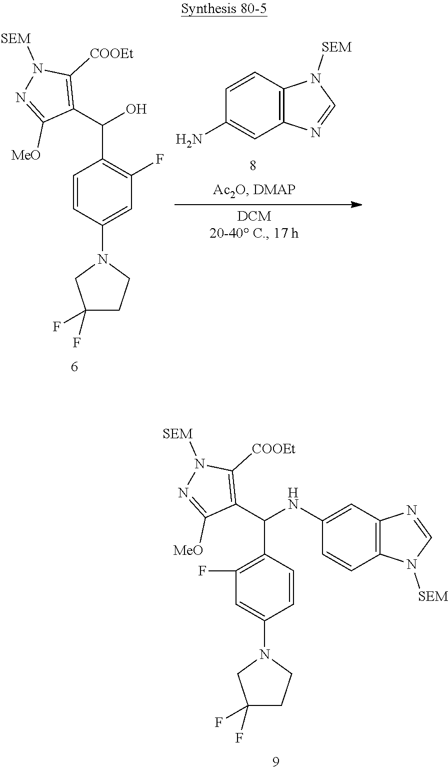

To a mixture of compound 6 (450.00 mg, 849.66 μmol, 1 eq) and DMAP (311.41 mg, 2.55 mmol, 3 eq) in DCM (1 mL) was added Ac₂O (130.11 mg, 1.27 mmol, 119.37 μL, 1.5 eq) at 20° C. under N₂. The mixture was stirred at 20° C. for 1 hr. Then compound 13A (447.62 mg, 1.70 mmol, 2 eq) was added, the reaction mixture was stirred at 40° C. for 16 hrs. The reaction mixture was concentrated in vacuum, the residue was purified by silica column chromatography (petroleum ether:ethyl acetate=10:1 to 0:1) to give compound 9 (430 mg, 543.73 μmol, 63.99% yield, 98% purity) as colorless oil.

LCMS: RT=1.675 min, m/z=766.1 (M+1)⁺.

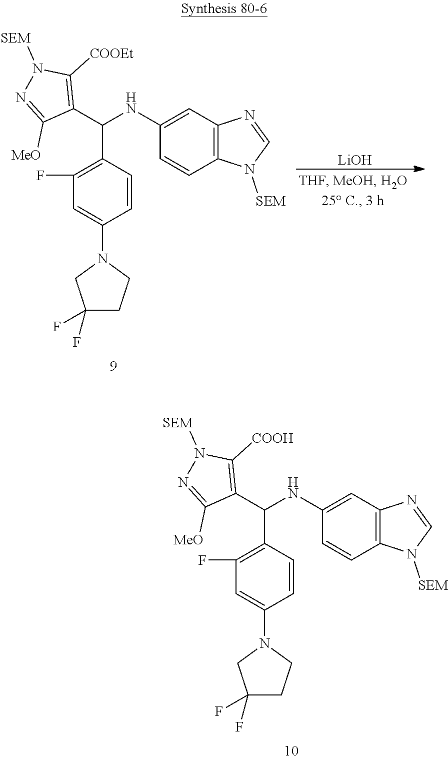 [SMILES: CCOC(=O)c1c(C(Nc2ccc3c(c2)ncn3COCC[Si](C)(C)C)c2ccc(N3CCC(F)(F)C3)cc2F)c(OC)nn1COCC[Si](C)(C)C ; "LiOH" ; COc1nn(COCC[Si](C)(C)C)c(C(=O)O)c1C(Nc1ccc2c(c1)ncn2COCC[Si](C)(C)C)c1ccc(N2CCC(F)(F)C2)cc1F]

To a solution of compound 9 (430.00 mg, 543.73 μmol, 98% purity, 1 eq) in THF (4 mL), MeOH (2 mL) and H₂O (2 mL) was added LiOH (39.07 mg, 1.63 mmol, 3 eq). The mixture was stirred at 25° C. for 3 hrs. TLC (dichloromethane:methanol=10:1; R_{f}=0.31) showed there was no compound 9 remained and one main new spot was detected. The mixture was adjusted pH to about 7 with 1 M HCl. The resulting mixture was extracted with EtOAc (5 mL*3). The combined organic phase was dried over Na₂SO₄, filtered and concentrated at reduced pressure to give compound 10 (0.39 g, crude) as yellow solid.

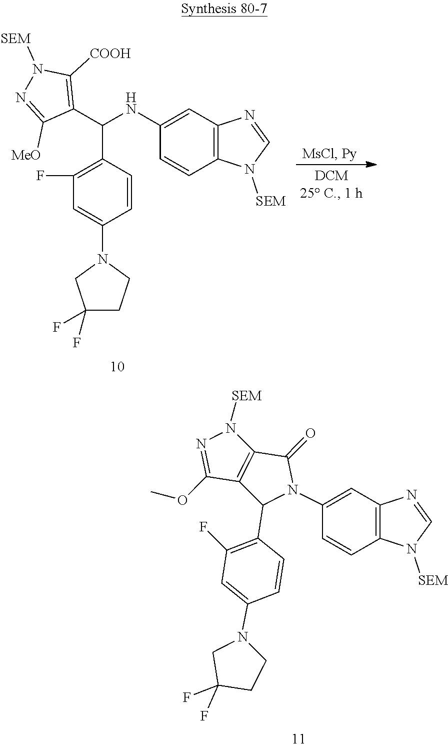

To a solution of compound 10 (390.00 mg, 522.11 μmol, 1 eq) in DCM (5 mL) was added Py (82.60 mg, 1.04 mmol, 84.28 μL, 2 eq) and MsCl (89.71 mg, 783.17 μmol, 60.62 μL, 1.5 eq) at 0° C. The mixture was stirred at 25° C. for 1 hr. The mixture was quenched with H₂O (5 mL) and extracted with DCM (5 mL*3). The organic layers were washed with brine (10 mL*2), dried over Na₂SO₄ and concentrated under vacuum to give a residue, the residue was purified by silica gel chromatography (silica gel, petroleum ether:ethyl acetate=10:1 to 0:1) to give compound 11 (0.16 g, 219.49 μmol, 42.04% yield) as yellow oil.

LCMS: RT=1.219 min, m/z=729.3 (M+1)⁺.

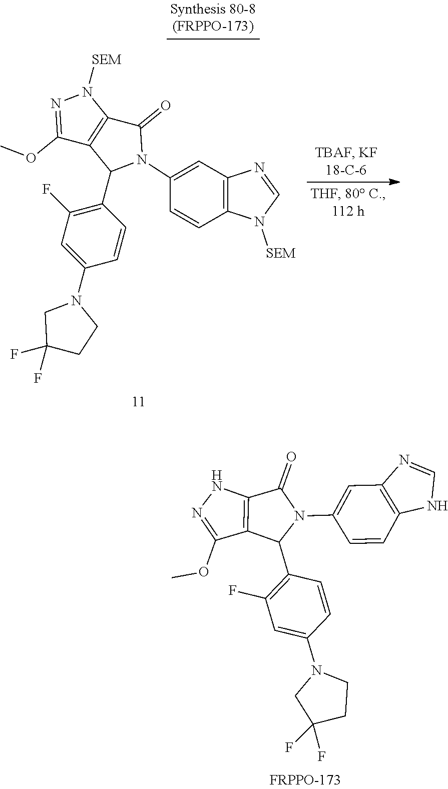 [SMILES: COc1nn(COCC[Si](C)(C)C)c2c1C(c1ccc(N3CCC(F)(F)C3)cc1F)N(c1ccc3c(c1)ncn3COCC[Si](C)(C)C)C2=O ; COc1n[nH]c2c1C(c1ccc(N3CCC(F)(F)C3)cc1F)N(c1ccc3[nH]cnc3c1)C2=O]

To a mixture of compound 11 (50 mg, 68.59 μmol, 1 eq), 18-C-6 (90.65 mg, 342.96 μmol, 5 eq) and TBAF (1 M, 137.18 μL, 2 eq) in THF (1 mL) was added KF (19.92 mg, 342.96 μmol, 8.03 μL, 5 eq). The mixture was stirred at 80° C. for 112 hrs. The mixture was diluted with EtOAc (20 mL) and the resulting mixture was washed with water (10 mL*5), brine (10 mL*3), dried over Na₂SO₄, filtered and concentrated at reduced pressure to give a residue. The residue was purified by prep-HPLC (column: Waters Xbridge 150*25 mm*5 μm; mobile phase: [water (10 mM NH₄HCO₃)-ACN]; B %: 18%-48%, 9 min) to give FRPPO-173 (1.15 mg, 2.38 μmol, 2.17% yield, 97% purity) as white solid.

¹H NMR (400 MHz, CD₃CN) δ=8.09 (s, 1H), 7.63 (d, J=1.7 Hz, 1H), 7.59-7.53 (m, 1H), 7.29 (dd, J=2.0, 8.6 Hz, 1H), 7.00 (t, J=8.7 Hz, 1H), 6.32 (s, 1H), 6.24 (dd, J=2.5, 8.9 Hz, 1H), 6.16 (dd, J=2.2, 13.7 Hz, 1H), 3.77 (s, 3H), 3.56 (t, J=13.1 Hz, 2H), 3.40 (t, J=7.3 Hz, 2H), 2.44-2.39 (m, 2H).

LCMS: RT=0.699 min. m/z=469.1 (M+1)⁺.

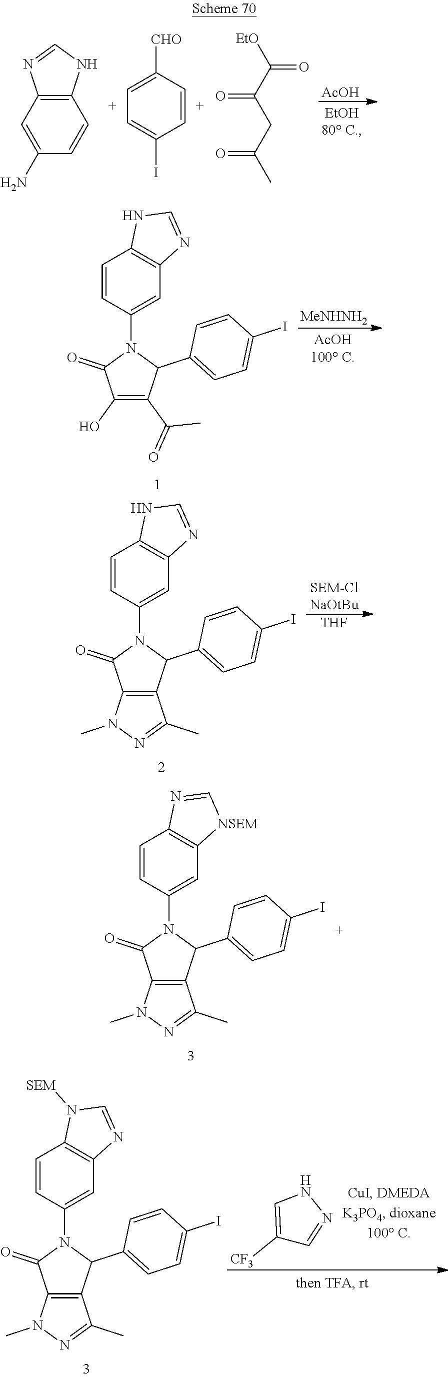

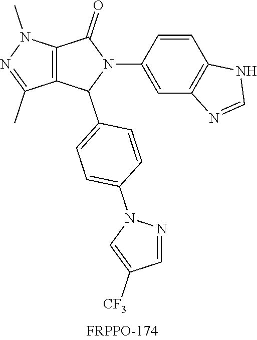 [SMILES: Cc1nn(C)c2c1C(c1ccc(-n3cc(C(F)(F)F)cn3)cc1)N(c1ccc3[nH]cnc3c1)C2=O]

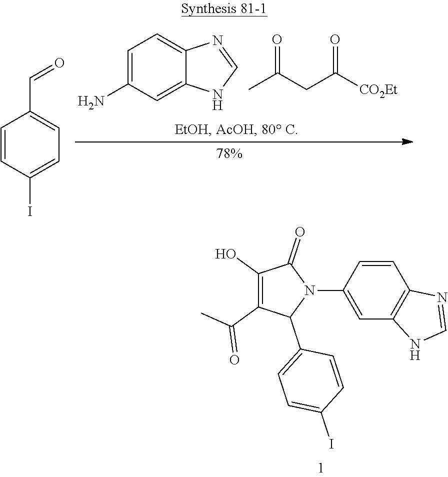

A solution of 4-iodobenzaldehyde (1.74 g, 7.52 mmol) and 1H-benzo[d]imidazol-6-amine (1.0 g, 1 eq.) in ethanol (10 mL) and acetic acid (0.5 mL) was heated at 80° C. for 20 mins. Next, ethyl 2,4-dioxopentanoate (1.05 mL, 1 eq.) was added dropwise, and the reaction heated for a further 2 h. The reaction was cooled to 0° C., and the product 1 filtered and collected as a dark green solid (2.7 g, 78%).

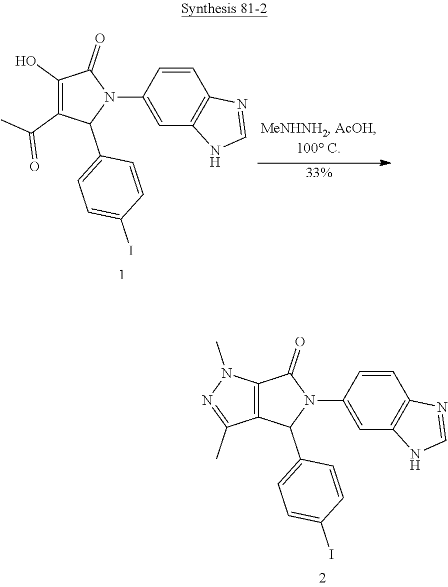 [SMILES: CC(=O)C1=C(O)C(=O)N(c2ccc3nc[nH]c3c2)C1c1ccc(I)cc1 ; Cc1nn(C)c2c1C(c1ccc(I)cc1)N(c1ccc3nc[nH]c3c1)C2=O]

To a solution of 1 (2.7 g, 5.88 mmol) in acetic acid (10 mL) was added methylhydrazine (619 μL, 2 eq.) dropwise. The reaction was heated to 100° C. for 3 h. The reaction was quenched with ice and sat. NaHCO₃ and extracted with ethyl acetate. The combined organic layers were washed with brine, dried over sodium sulfate and concentrated in vacuo. The crude product was purified by FCC to give the desired product as a tan solid (900 mg, 33%). The desired regioisomer 2 elutes first in 5% MeOH/DCM on silica gel chromatography.

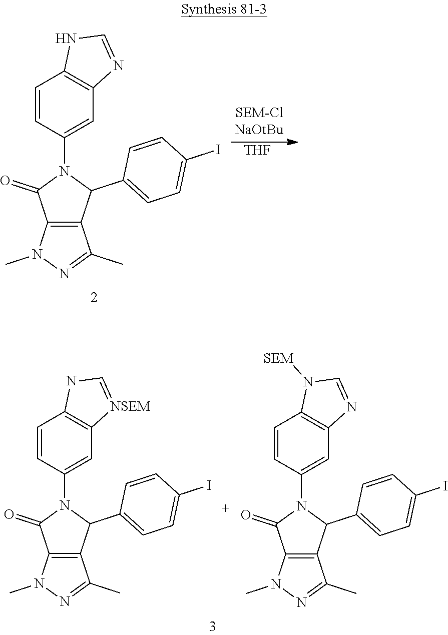

To a solution of 2 (850 mg, 1.81 mmol) in THF (10 mL) was added NaOtBu (209 mg, 1.2 eq.) followed by SEMCl (385 μL, 1.2 eq.). The reaction was stirred for 1 h at rt, and quenched with sat. NaHCO₃. The reaction was extracted with ethyl acetate, dried over sodium sulfate and concentrated in vacuo. The crude product was purified by FCC to give 3 as a tan solid (900 mg, 83%). The product was isolated as mixture of N-SEM regioisomers.

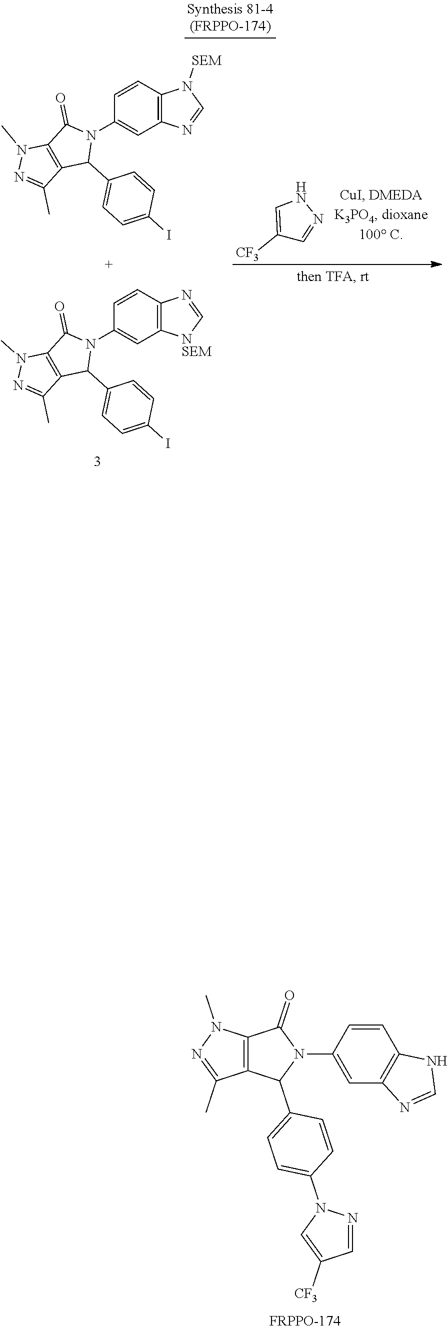 [SMILES: Cc1nn(C)c2c1C(c1ccc(I)cc1)N(c1ccc3c(c1)ncn3COCC[Si](C)(C)C)C2=O ; FC(F)(F)c1cn[nH]c1 ; Cc1nn(C)c2c1C(c1ccc(I)cc1)N(c1ccc3ncn(COCC[Si](C)(C)C)c3c1)C2=O ; Cc1nn(C)c2c1C(c1ccc(-n3cc(C(F)(F)F)cn3)cc1)N(c1ccc3[nH]cnc3c1)C2=O]

A solution of 3 (30 mg, 0.05 mmol), CuI (4.8 mg, 0.5 eq.), DMEDA (5.4 μL, 1 eq.), 4-(trifluoromethyl)-1H-pyrazole (13.6 mg, 2.0 eq.) and K₃PO₄ (32 mg, 3eq.) in dioxane was heated to 100° C. overnight. The reaction was quenched with sat. NaHCO₃ and extracted with DCM. The combined organic layers were dried over sodium sulfate and concentrated in vacuo. The crude reaction was dissolved in TFA (1 mL) and stirred at room temperature for 6 h. The reaction was quenched with sat. Na₂CO₃ and extracted with DCM. The organic layers were dried over sodium sulfate and concentrated in vacuo. The crude product was purified by silica gel chromatography followed by reverse phase LC to give FRPPO-174 as an off white solid (4.3 mg, 18%, 96.01% purity).

¹H NMR (400 MHz, DMSO-d₆) δ 12.45 (s, 1H), 9.31-8.96 (m, 1H), 8.16 (d, J=12.2 Hz, 2H), 7.76 (d, J=8.5 Hz, 2H), 7.71 (d, J=19.8 Hz, 1H), 7.62-7.17 (m, 3H), 6.48 (d, J=6.0 Hz, 1H), 3.97 (s, 3H), 1.97 (s, 3H).

LC: Gradient 5% to 95% MeCN:H₂O (0.1% HCOOH), KromegaSil OSD-H, 7.21 min.

LRMS: C₂₄H₁₉F₃N₇O requires 478.160. Found 478.174.

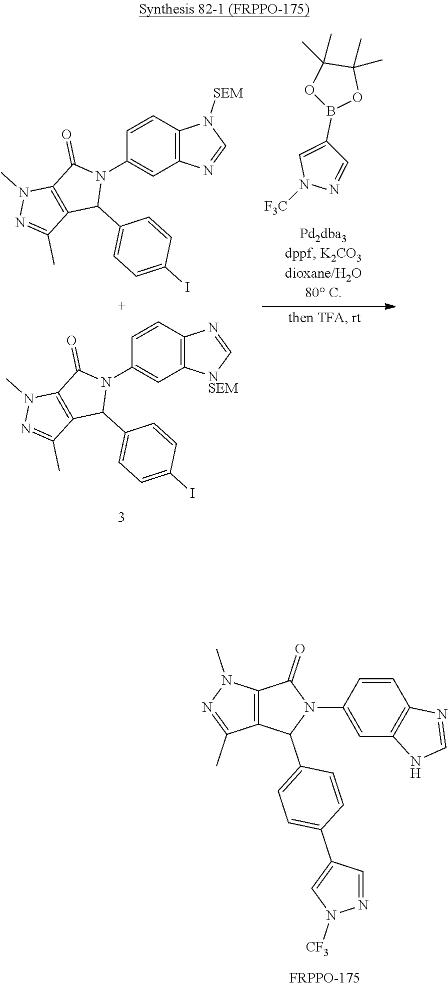 [SMILES: Cc1nn(C)c2c1C(c1ccc(I)cc1)N(c1ccc3c(c1)ncn3COCC[Si](C)(C)C)C2=O ; CC1(C)OB(c2cnn(C(F)(F)F)c2)OC1(C)C ; Cc1nn(C)c2c1C(c1ccc(I)cc1)N(c1ccc3ncn(COCC[Si](C)(C)C)c3c1)C2=O ; Cc1nn(C)c2c1C(c1ccc(-c3cnn(C(F)(F)F)c3)cc1)N(c1ccc3nc[nH]c3c1)C2=O]

To a scintillation vial was charged 3 (30 mg, 0.05 mmol), 4-(4,4,5,5-tetramethyl-1,3,2-dioxaborolan-2-yl)-1-(trifluoromethyl)-1H-pyrazole (26.2 mg, 2.0 eq.), Pd₂dba₃ (11.4 mg, 0.25 eq.), dppf (13.9 mg, 0.5 eq.), potassium carbonate (21 mg, 3.0 eq.), dioxane (0.8 mL) and water (0.2 mL). The reaction was heated to 85° C. for 5 h. The crude product was diluted with sat. NaHCO₃ and extracted with DCM. The organic layers were dried over sodium sulfate and concentrated in vacuo. The crude was dissolved in TFA (1 mL) and stirred at rt for 6 h. The reaction was quenched with sat. NaHCO₃ and extracted with DCM. The combined organic layers were dried over sodium sulfate and concentrated in vacuo. The crude product was purified by silica gel chromatography followed by reverse phase LC to give FRPPO-175 as an off white solid (4.9 mg, 21%, 95.29% purity).

¹H NMR (400 MHz, Acetonitrile-d₃) δ 10.38 (s, 1H), 8.30 (s, 1H), 8.07 (s, 1H), 7.98 (d, J=2.3 Hz, 1H), 7.78-7.44 (m, 4H), 7.41-7.04 (m, 3H), 6.14 (d, J=5.0 Hz, 1H), 3.99 (s, 3H), 1.99 (s, 3H).

LC: Gradient 5% to 95% MeCN:H₂O (0.1% HCOOH), KromegaSil OSD-H, 7.70 min.

LRMS: C₂₄H₁₉F₃N₇O requires 478.160. Found 478.009.

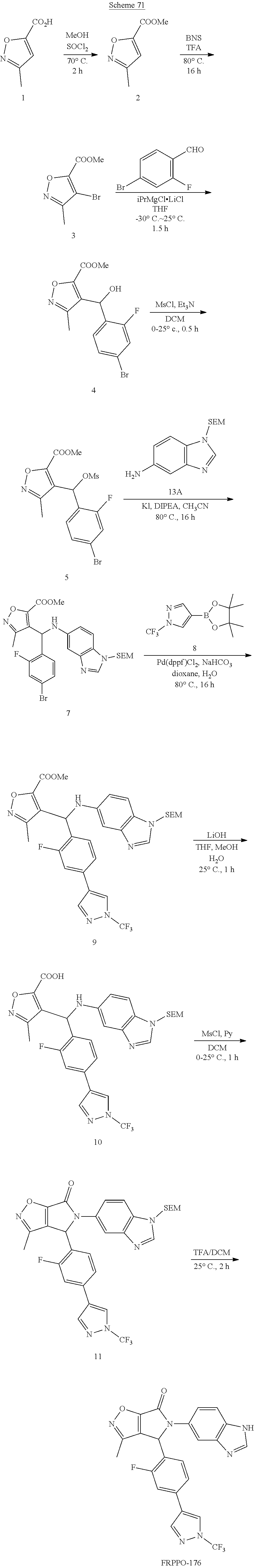

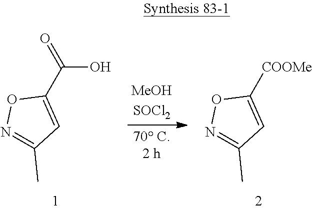

To a mixture of compound 1 (5 g, 39.34 mmol, 1 eq) in MeOH (30 mL) was added SOCl₂ (5.62 g, 47.21 mmol, 3.42 mL, 1.2 eq) slowly. The mixture was stirred at 70° C. for 2 hrs. TLC (Dichloromethane:Methanol=10:1) showed 3-methylisoxazole-5-carboxylic acid was consumed and one spot was detected. The reaction mixture was concentrated under vacuum to give compound 2 (5.45 g, crude) as white solid.

¹H NMR (400 MHz, DMSO-d₆) δ=7.16 (s, 1H), 3.89 (s, 3H), 2.31 (s, 3H).

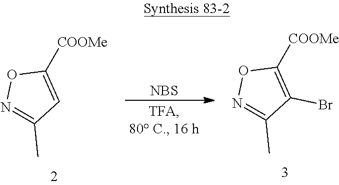

To a mixture of compound 2 (10 g, 70.86 mmol, 1 eq) in TFA (100 mL) was added NBS (15.13 g, 85.03 mmol, 1.2 eq) at 25° C. The mixture was stirred at 80° C. for 16 hrs. TLC (petroleum ether:ethyl acetate=5:1) showed methyl 3-methylisoxazole-5-carboxylate was consumed and one new spot was detected. The reaction mixture was concentrated under vacuum to give a residue. The residue was diluted with EtOAc (300 mL), washed with saturated NaHCO₃ aqueous solution (100 mL) and brine (50 ml*2), the organic layer was dried over Na₂SO₄, filtered and the filtrate was concentrated in vacuum to give a residue. The residue was purified by silica gel chromatography (100-200 mesh silica gel, petroleum ether:ethyl acetate=10:1, 5:1) to give compound 3 (13.3 g, 85% yield) as white solid.

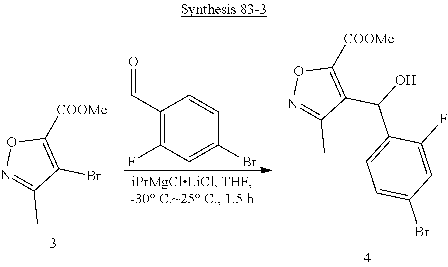

To a mixture of compound 3 (12.3 g, 55.90 mmol, 1 eq) in THF (100 mL) was added i-PrMgCl—LiCl (1.3 M, 51.60 mL, 1.2 eq) slowly at −30° C. under N₂. The mixture was stirred at −30° C. for 15 min, then 4-bromo-2-fluoro-benzaldehyde (11.35 g, 55.90 mmol, 1 eq) was added and the mixture was stirred at 25° C. for 1 hr. TLC (petroleum ether:ethyl acetate=5:1) showed methyl 4-iodo-3-methyl-isoxazole-5-carboxylate was consumed and one new spot was detected. The mixture was quenched with saturated aqueous NH₄Cl (200 mL). The resulting solution was extracted with EtOAc (200 mL). The organic layers was washed with brine (100 ml*2) and dried over Na₂SO₄, and concentrated under vacuum to give a residue. The residue was purified by column chromatography on silica gel eluted (petroleum ether:ethyl acetate=20:1 to 10:1) to give compound 4 (6.8 g, 34.4% yield) as yellow oil.

¹H NMR (400 MHz, CHLOROFORM-d) δ=7.49-7.38 (m, 1H), 7.33 (dd, J=1.7, 8.4 Hz, 1H), 7.21 (dd, J=1.8, 9.9 Hz, 1H), 6.22 (d, J=8.4 Hz, 1H), 4.42 (d, J=8.4 Hz, 1H), 4.01 (s, 3H), 2.26 (s, 3H).

LCMS: RT=0.917 min, m/z=344.0 (M+1)⁺.

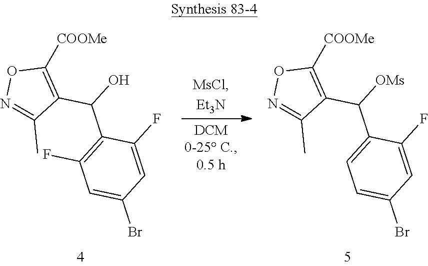 [SMILES: COC(=O)c1onc(C)c1C(O)c1c(F)cc(Br)cc1F ; COC(=O)c1onc(C)c1C(OS(C)(=O)=O)c1ccc(Br)cc1F]

To a solution of compound 4 (1 g, 2.91 mmol, 1 eq) in DCM (10 mL) was added TEA (882.12 mg, 8.72 mmol, 1.21 mL, 3 eq) and MsCl (399.44 mg, 3.49 mmol, 269.89 μL, 1.2 eq) at 0° C., the mixture was stirred at 25° C. for 0.5 hr. The mixture was quenched with H₂O (20 mL) and extracted with EtOAc (100 mL). The organic layers was washed with brine (50 ml*2) and dried over Na₂SO₄, and concentrated under vacuum to give a residue to give compound 5 (1.3 g, crude) as yellow oil.

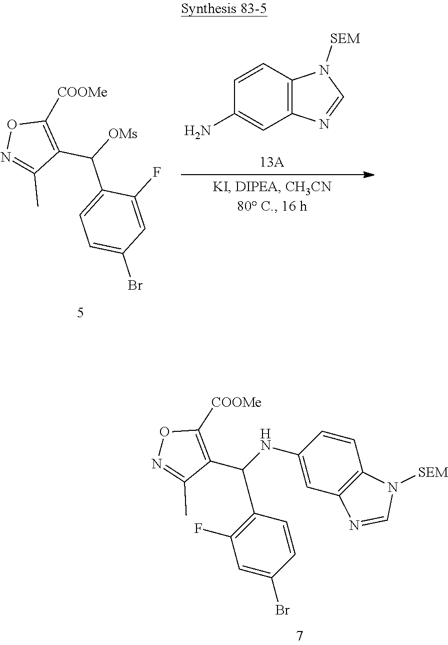 [SMILES: COC(=O)c1onc(C)c1C(OS(C)(=O)=O)c1ccc(Br)cc1F ; C[Si](C)(C)CCOCn1cnc2cc(N)ccc21 ; COC(=O)c1onc(C)c1C(Nc1ccc2c(c1)ncn2COCC[Si](C)(C)C)c1ccc(Br)cc1F]

To a mixture of compound 5 (1.3 g, 3.08 mmol, 1 eq) and compound 13A (Scheme 68 and Synthesis 79-2) (892.13 mg, 3.39 mmol, 1.1 eq) in CH3CN (5 mL) were added KI (51.11 mg, 307.89 μmol, 0.1 eq) and DIEA (795.84 mg, 6.16 mmol, 1.07 mL, 2 eq). The mixture was stirred at 80° C. for 16 hrs. The mixture was concentrated under vacuum to give a residue. The residue was purified by column chromatography on silica gel eluted (petroleum ether:ethyl acetate=5:1 to 1:1) to give compound 7 (530 mg, 29% yield) as yellow oil.

LCMS: RT=0.928 min, m/z=589.1 (M+1)⁺.

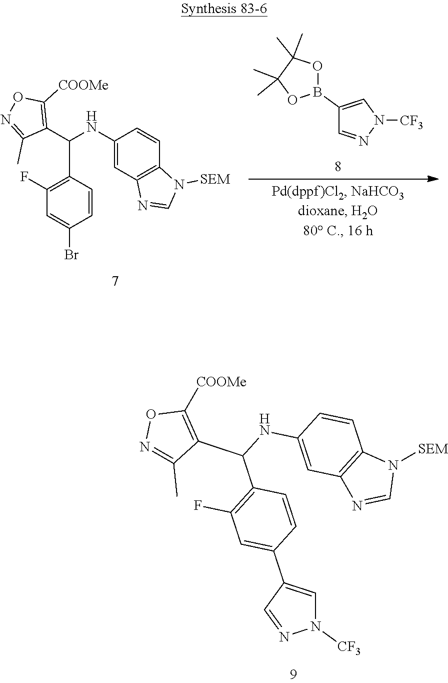 [SMILES: COC(=O)c1onc(C)c1C(Nc1ccc2c(c1)ncn2COCC[Si](C)(C)C)c1ccc(Br)cc1F ; CC1(C)OB(c2cnn(C(F)(F)F)c2)OC1(C)C ; COC(=O)c1onc(C)c1C(Nc1ccc2c(c1)ncn2COCC[Si](C)(C)C)c1ccc(-c2cnn(C(F)(F)F)c2)cc1F]

To a mixture of compound 7 (530 mg, 899.03 μmol, 1 eq) and compound 8 (235.58 mg, 899.03 μmol, 1 eq) in dioxane (5 mL) and water (1 mL) was added Pd(dppf)Cl₂ (65.78 mg, 89.90 μmol, 0.1 eq) and NaHCO₃ (151.05 mg, 1.80 mmol, 69.93 μL, 2 eq) under N₂. The mixture was stirred at 80° C. for 16 hrs. The mixture was diluted with EtOAc (30 mL), washed with brine (10 mL*2) and dried over Na₂SO₄, and concentrated under vacuum to give a residue. The residue was purified by silica gel chromatography (300-400 mesh silica gel, petroleum ether:ethyl acetate=5:1 to 1:1) to give compound 9 (250 mg, crude) as yellow oil. (There was compound 7 contained in the product, compound 7 and compound 9 were in the same peak in LCMS).

LCMS: RT=0.849 min. m/z=645.3 (M+1)⁺.

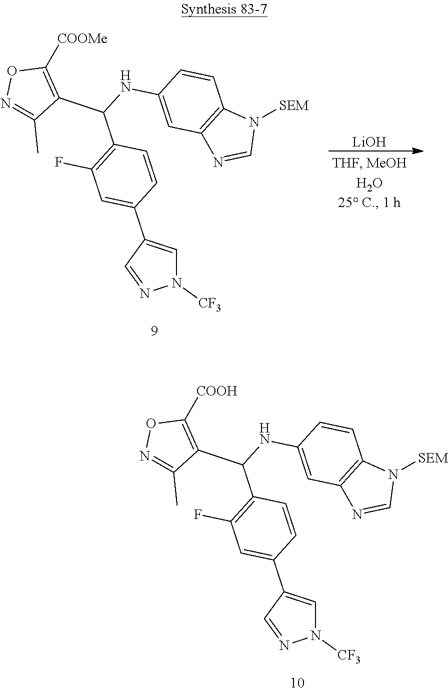 [SMILES: COC(=O)c1onc(C)c1C(Nc1ccc2c(c1)ncn2COCC[Si](C)(C)C)c1ccc(-c2cnn(C(F)(F)F)c2)cc1F ; "LiOH" ; Cc1noc(C(=O)O)c1C(Nc1ccc2c(c1)ncn2COCC[Si](C)(C)C)c1ccc(-c2cnn(C(F)(F)F)c2)cc1F]

To a solution of compound 9 (230 mg, 356.76 μmol, 1 eq) in THF (4 mL), H₂O (1 mL) and MeOH (2 mL) was added LiOH (42.72 mg, 1.78 mmol, 5 eq) and. The mixture was stirred at 25° C. for 1 hr. The reaction mixture was adjusted pH=5 with 1 M HCl and extracted with EtOAc (30 mL), the organic layers was washed with brine (10 mL) and dried over Na₂SO₄, and concentrated under vacuum to give a residue to give compound 10 (190 mg, crude) as yellow solid.

LCMS: RT=0.816 min, m/z=631.1 (M+1)⁺.

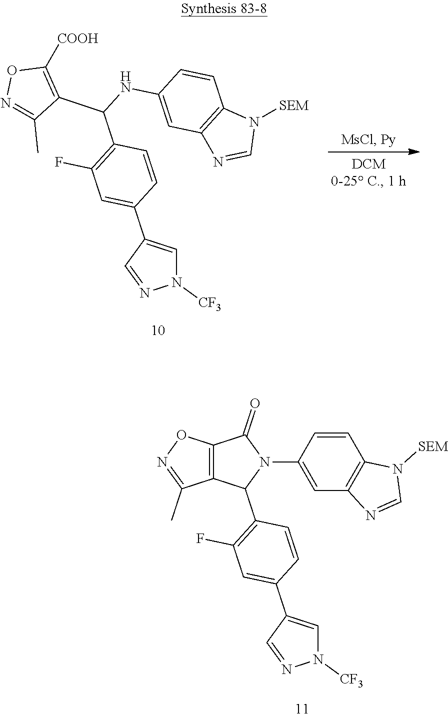 [SMILES: Cc1noc(C(=O)O)c1C(Nc1ccc2c(c1)ncn2COCC[Si](C)(C)C)c1ccc(-c2cnn(C(F)(F)F)c2)cc1F ; Cc1noc2c1C(c1ccc(-c3cnn(C(F)(F)F)c3)cc1F)N(c1ccc3c(c1)ncn3COCC[Si](C)(C)C)C2=O]

To a solution of compound 10 (190 mg, 301.27 μmol, 1 eq in DCM (5 mL) was added Py (47.66 mg, 602.54 μmol, 48.63 μL, 2 eq) and MsCl (51.77 mg, 451.90 μmol, 34.98 μL, 1.5 eq) at 0° C. The mixture was stirred at 25° C. for 1 hr. The mixture was quenched with H₂O and extracted with DCM (20 mL). The organic layers was washed with brine and dried over Na₂SO₄, and concentrated under vacuum to give a residue. The residue was purified by prep-TLC (petroleum ether:ethyl acetate=1:1, R_{f}=0.14) to give compound 11 (30 mg, crude) as yellow oil.

LCMS: RT=0.963 min. m/z=613.4 (M+1)⁺.

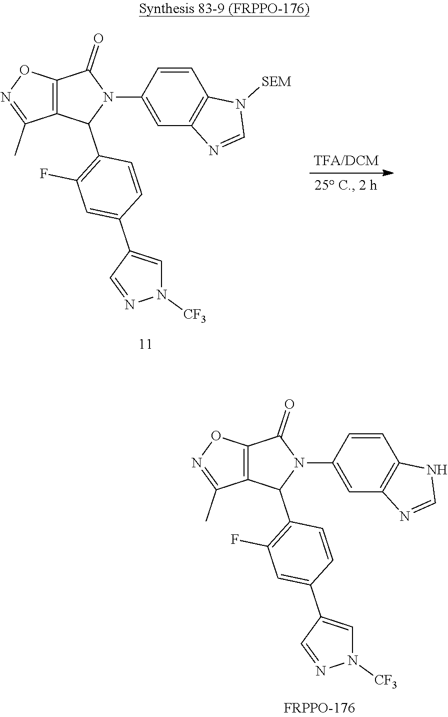 [SMILES: Cc1noc2c1C(c1ccc(-c3cnn(C(F)(F)F)c3)cc1F)N(c1ccc3c(c1)ncn3COCC[Si](C)(C)C)C2=O ; Cc1noc2c1C(c1ccc(-c3cnn(C(F)(F)F)c3)cc1F)N(c1ccc3[nH]cnc3c1)C2=O]

To a solution of compound 11 (20 mg, 32.65 μmol, 1 eq) in DCM (0.5 mL) was added TFA (770.00 mg, 0.5 mL), the mixture was stirred at 25° C. for 2 hrs. LCMS showed compound 11 was consumed and one peak with desired mass was detected. The mixture was concentrated under vacuum to give a residue. The residue was purified by prep-HPLC (column: Phenomenex Gemini-NX C18 75*30 mm*3 μm; mobile phase: [water (10 mM NH₄HCO₃)-ACN]; B %: 24%-54%, 8 min). The combined fractions were lyophilized to give FRPPO-176 (2.8 mg, 100% purity).

¹H NMR (400 MHz, ACETONITRILE-d₃) δ=10.46 (s, 1H), 8.36 (s, 1H), 8.09 (s, 1H), 8.01 (s, 1H), 7.67 (s, 1H), 7.64-7.49 (m, 1H), 7.36-7.25 (m, 4H), 6.46 (br s, 1H), 2.18 (s, 3H).

LCMS: RT=0.734 min. m/z=483.0 (M+1)⁺.

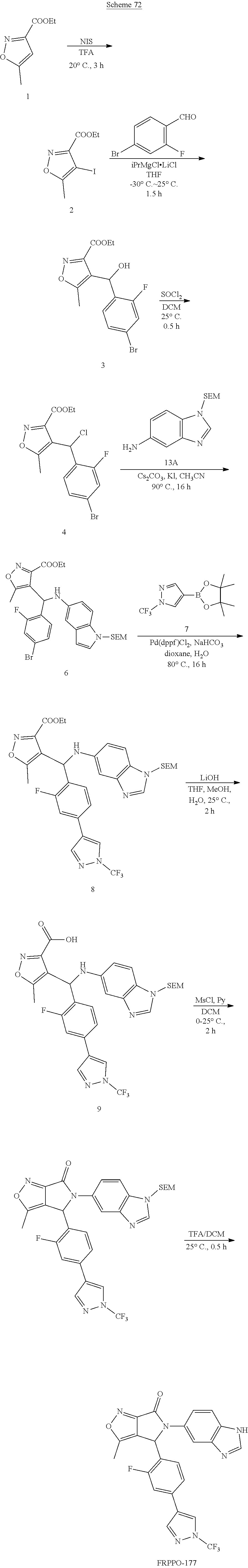

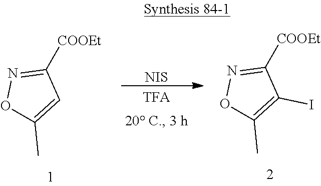

To a mixture of ethyl 5-methylisoxazole-3-carboxylate 1 (3 g, 19.34 mmol, 1 eq) in TFA (30 mL) was added NIS (4.79 g, 21.27 mmol, 1.1 eq) in one portion at 20° C. under N₂. The mixture was stirred at 20° C. for 3 hrs. TLC (petroleum ether:ethyl acetate=5:1) showed there was no ethyl 5-methylisoxazole-3-carboxylate remained and one main spot was detected. The mixture was concentrated at reduced pressure to give a residue, the residue was diluted with ethyl acetate (150 mL), the organic layer was washed with saturated NaHCO₃ aqueous solution (50 mL*2), saturated NaHCO₃ aqueous solution (50 mL), dried over Na₂SO₄, filtered and the filtrate was concentrated in vacuum, the residue was purified by silica gel chromatography (100-200 mesh silica gel, petroleum ether:ethyl acetate=50:1, 30:1) to give compound 2 (4.6 g, 16.37 mmol, 84.65% yield) as white solid.

¹H NMR (400 MHz, CHLOROFORM-d) δ=4.47 (q, J=7.1 Hz, 2H), 2.56 (s, 3H), 1.44 (t, J=7.2 Hz, 3H).

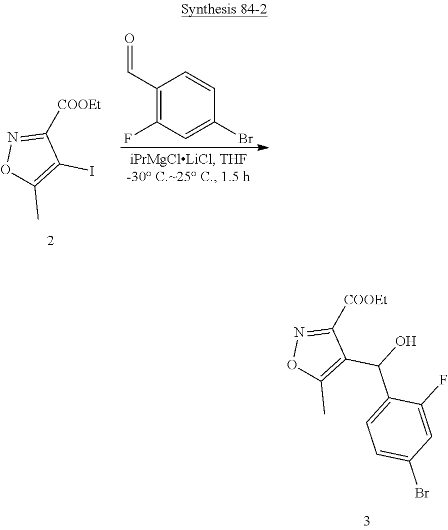 [SMILES: CCOC(=O)c1noc(C)c1I ; O=Cc1ccc(Br)cc1F ; CCOC(=O)c1noc(C)c1C(O)c1ccc(Br)cc1F]

To a mixture of compound 2 (2.1 g, 7.47 mmol, 1 eq) in THF (30 mL) was added i-PrMgCl—LiCl (1.3 M, 6.90 mL, 1.2 eq) slowly at −30° C. under N₂. The mixture was stirred at −30° C. for 15 min, then 4-bromo-2-fluoro-benzaldehyde (1.52 g, 7.47 mmol, 1 eq) was added and the mixture was stirred at 25° C. for 1 hr. TLC (petroleum ether:ethyl acetate=5:1) showed ethyl 4-iodo-3-methoxy-2-(2-trimethylsilylethoxymethyl)-3,4-dihydropyrazole-5-carboxylate was consumed and two new spots were detected. The mixture was quenched with saturated aqueous NH₄Cl (20 mL). The resulting solution was extracted with EtOAc (50 mL). The organic layers was washed with brine and dried over Na₂SO₄, and concentrated under vacuum to give a residue. The residue was purified by column chromatography on silica gel (petroleum ether:ethyl acetate=20:1 to 5:1) to give compound 3 (1.25 g, 3.49 mmol, 46.71% yield) as yellow oil.

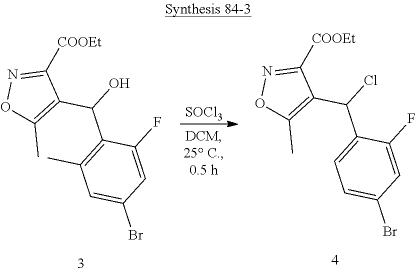

To a mixture of compound 3 (500 mg, 1.40 mmol, 1 eq) in DCM (10 mL) was added Py (331.28 mg, 4.19 mmol, 338.04 μL, 3 eq), then SOCl₂ (498.26 mg, 4.19 mmol, 303.81 μL, 3 eq) was added under N₂. The mixture was stirred at 25° C. for 0.5 hr. The reaction was monitored by TLC (petroleum ether:ethyl acetate=5:1), compound 3 was consumed, a main new spot was observed. The reaction mixture was diluted with DCM (30 mL), the organic layer was washed with water (20 mL), then washed with saturated NaHCO₃ aqueous solution (20 mL*2), the organic layer was dried over sodium sulfate, filtered and the filtrate was concentrated in vacuum to give compound 4 (520 mg, crude) as yellow oil, which was used to next step without purification.

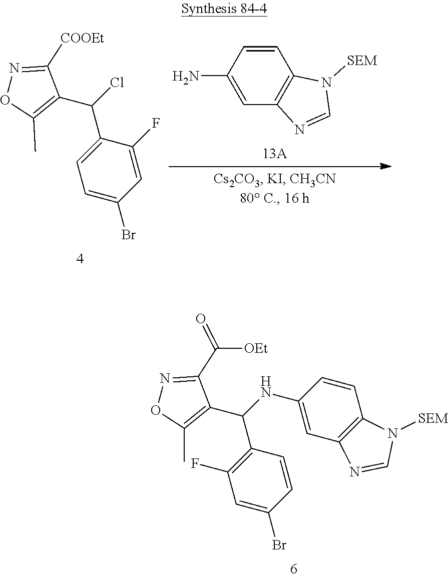 [SMILES: CCOC(=O)c1noc(C)c1C(Cl)c1ccc(Br)cc1F ; C[Si](C)(C)CCOCn1cnc2cc(N)ccc21 ; CCOC(=O)c1noc(C)c1C(Nc1ccc2c(c1)ncn2COCC[Si](C)(C)C)c1ccc(Br)cc1F]

To a mixture of compound (0.2 g, 1.3 mmol, 1eq) (crude) and compound 13A (Scheme 68 and Synthesis 79-2) (363.71 mg, 1.38 mmol, 1 eq) in acetonitrile (10 mL) were added KI (22.92 mg, 138.08 μmol, 0.1 eq), DIPEA (356.91 mg, 2.76 mmol, 481.01 μL, 2 eq). The mixture was stirred at 80° C. for 16 hrs. TLC (ethyl acetate:methanol=30:1) showed compound 4 was consumed and one new spot was detected. The reaction mixture was concentrated, the residue was purified by silica gel chromatography (petroleum ether:ethyl acetate=5:1 to 1:1) to give compound 6 (620 mg, 1.03 mmol, 74.40% yield) as yellow oil.

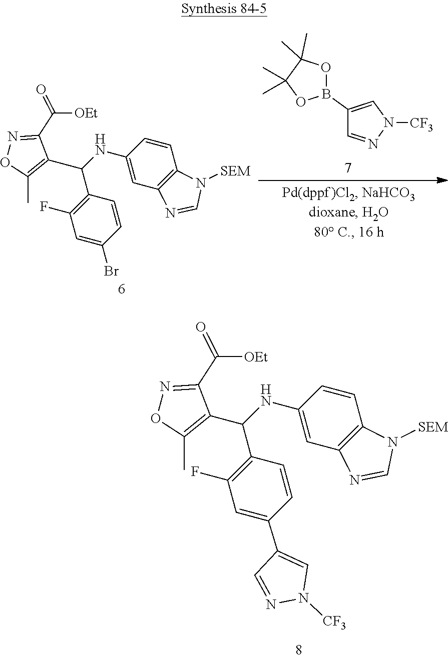 [SMILES: CCOC(=O)c1noc(C)c1C(Nc1ccc2c(c1)ncn2COCC[Si](C)(C)C)c1ccc(Br)cc1F ; CC1(C)OB(c2cnn(C(F)(F)F)c2)OC1(C)C ; CCOC(=O)c1noc(C)c1C(Nc1ccc2c(c1)ncn2COCC[Si](C)(C)C)c1ccc(-c2cnn(C(F)(F)F)c2)cc1F]

To a mixture of compound 6 (300 mg, 497.06 μmol, 1 eq) and compound 7 (130.25 mg, 497.06 μmol, 1 eq) in dioxane (6 mL) and H₂O (1.5 mL) was added Pd(dppf)Cl₂ (36.37 mg, 49.71 μmol, 0.1 eq) and NaHCO₃ (83.52 mg, 994.11 μmol, 38.66 μL, 2 eq) under N₂. The mixture was stirred at 80° C. for 16 hrs. The mixture was extracted with EtOAc (30 mL). The organic layer was washed with brine (15 mL*2) and dried over Na₂SO₄, and concentrated under vacuum to give a residue. The residue was purified by column chromatography on silica gel eluted (petroleum ether:ethyl acetate=20:1 to 1:1) to give compound 8 (220 mg, 333.98 μmol, 67.19% yield) as yellow oil.

LCMS: RT=0.881 min, m/z=659.1 (M+1)⁺.

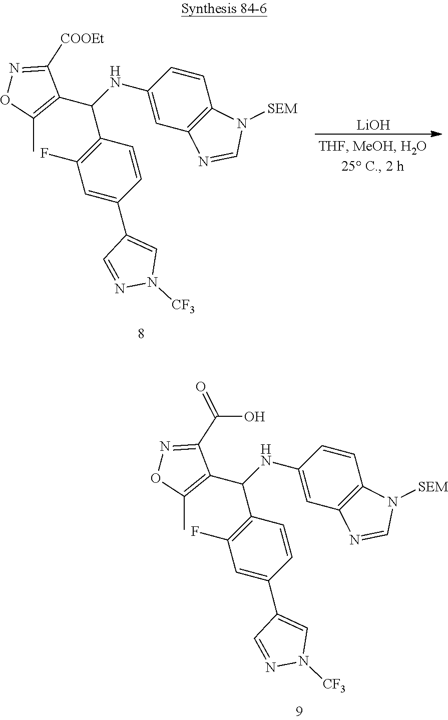 [SMILES: CCOC(=O)c1noc(C)c1C(Nc1ccc2c(c1)ncn2COCC[Si](C)(C)C)c1ccc(-c2cnn(C(F)(F)F)c2)cc1F ; "LiOH" ; Cc1onc(C(=O)O)c1C(Nc1ccc2c(c1)ncn2COCC[Si](C)(C)C)c1ccc(-c2cnn(C(F)(F)F)c2)cc1F]

To a solution of compound 8 (210 mg, 318.80 μmol, 1 eq) in THF (4 mL), MeOH (2 mL) and H₂O (1 mL) was added LiOH (38.17 mg, 1.59 mmol, 5 eq). The mixture was stirred at 25° C. for 2 hrs. The reaction mixture was adjusted pH=5 with 1 M HCl aqueous solution and extracted with EtOAc (30 mL), the organic layers was washed with brine (10 mL) and dried over Na₂SO₄, and concentrated under vacuum to give compound 9 (190 mg, crude) as yellow oil.

LCMS: RT=0.883 min. m/z=631.1 (M+1)⁺.

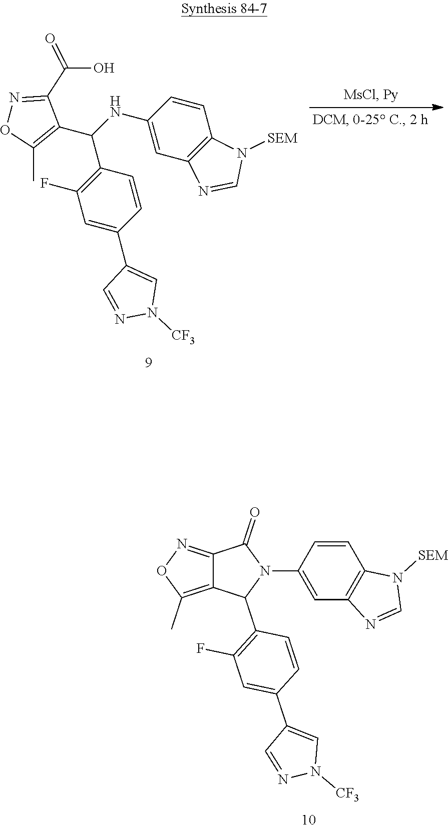 [SMILES: Cc1onc(C(=O)O)c1C(Nc1ccc2c(c1)ncn2COCC[Si](C)(C)C)c1ccc(-c2cnn(C(F)(F)F)c2)cc1F ; Cc1onc2c1C(c1ccc(-c3cnn(C(F)(F)F)c3)cc1F)N(c1ccc3c(c1)ncn3COCC[Si](C)(C)C)C2=O]

To a solution of compound 9 (190 mg, 301.27 μmol, 1 eq) in DCM (5 mL) was added MsCl (51.77 mg, 451.90 μmol, 34.98 μL, 1.5 eq) and Py (47.66 mg, 602.54 μmol, 48.63 μL, 2 eq) at 0° C. The mixture was stirred at 25° C. for 1 hr. LCMS showed compound 9 was remained and one peak with desired mass was detected. Mscl (51.77 mg, 451.90 μmol, 34.98 μL, 1.5 eq) and Py (47.66 mg, 602.54 μmol, 48.63 μL, 2 eq) was added at 0° C. The mixture was stirred at 25° C. for 1 hr. The mixture was quenched with H₂O and extracted with DCM (20 mL). The organic layers was washed with brine and dried over Na₂SO₄, and concentrated under vacuum to give a residue, the residue was purified by prep-TLC (petroleum ether:ethyl acetate=0:1) to give compound 10 (30 mg, 48.97 μmol, 16.25% yield) as yellow solid.

LCMS: RT=0.883 min, m/z=613.2 (M+1)⁺.

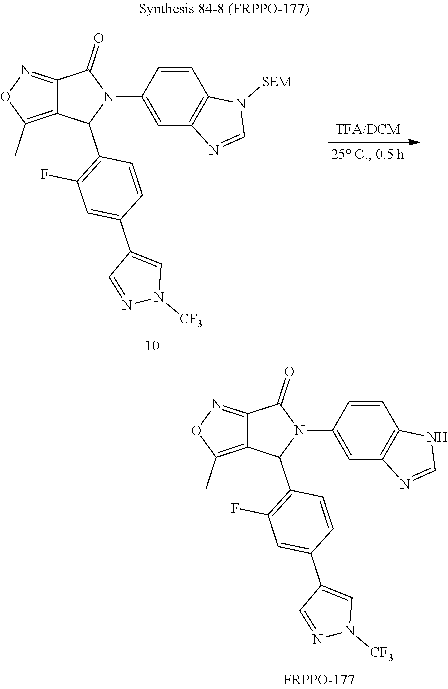 [SMILES: Cc1onc2c1C(c1ccc(-c3cnn(C(F)(F)F)c3)cc1F)N(c1ccc3c(c1)ncn3COCC[Si](C)(C)C)C2=O ; Cc1onc2c1C(c1ccc(-c3cnn(C(F)(F)F)c3)cc1F)N(c1ccc3[nH]cnc3c1)C2=O]

To a solution of compound 10 (30 mg, 48.97 μmol, 1 eq) in DCM (1 mL) was added TFA (1 mL) under N₂. The mixture was stirred at 25° C. for 0.5 hr. The reaction mixture was concentrated in vacuum, the residue was purified by prep-HPLC (column: Waters Xbridge 150*25 mm*5 μm; mobile phase: [water (10 mM NH₄HCO₃)-ACN]; B %: 25%-55%, 9 min) followed by lyophilization. To give FRPPO-177 (9.89 mg, 19.07 μmol, 38.94% yield, 93% purity) as yellow solid.

¹H NMR (400 MHz, CHLOROFORM-d) δ=14.47-14.28 (brs, 1H), 9.72-9.41 (brs, 1H), 8.18 (br d, J=1.2 Hz, 1H), 8.04 (s, 1H), 7.86 (s, 1H), 7.47-7.38 (m, 1H), 7.36-7.28 (m, 2H), 7.23-7.18 (m, 1H), 7.12 (br d, J=11.4 Hz, 1H), 6.61 (br s, 1H), 2.31 (s, 3H).

LCMS: RT=0.739 min, m/z=483.0 (M+1)⁺.

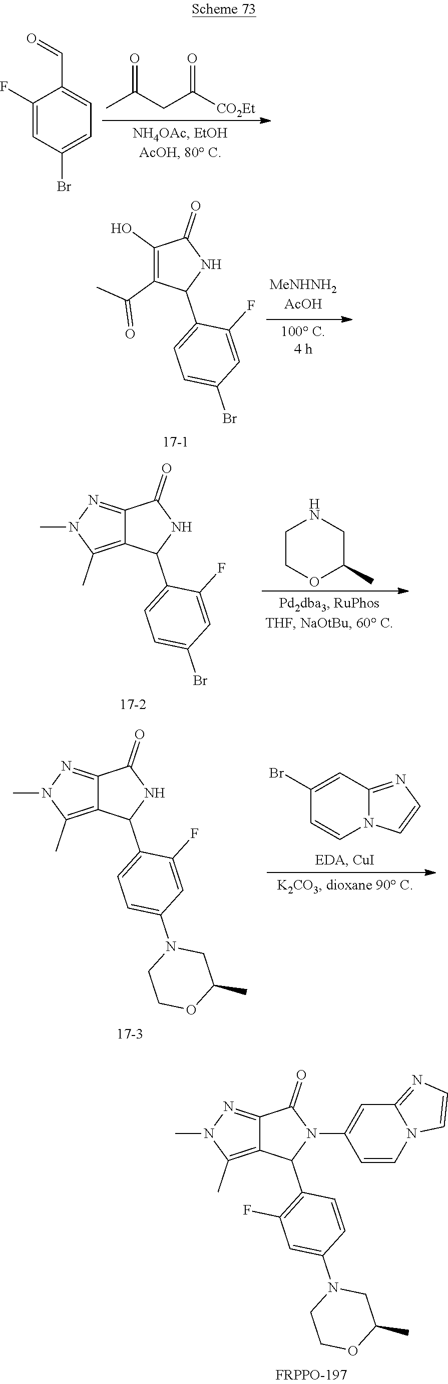

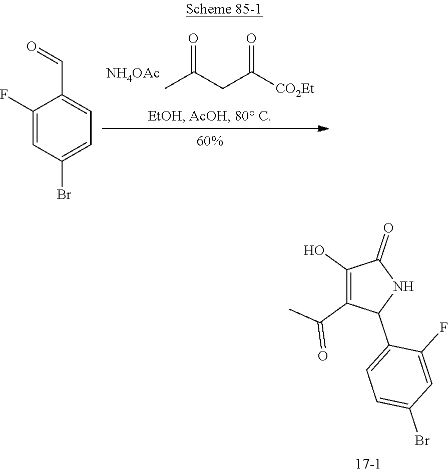

To a solution of 2-fluoro-4-bromobenzaldehyde (2.03 g, 10 mmol) in ethanol (20 mL) and acetic acid (0.5 mL) was added ammonium acetate (1.0 eq.) and the reaction heated to 80° C. for 30 mins. Ethyl 2,4-dioxopentanoate (1.40 mL, 1.0 eq.) was added dropwise and the reaction heated for a further 4 h. The reaction was cooled to 0° C. and filtered to give 17-1 as a brown solid (1.88 g, 60%).

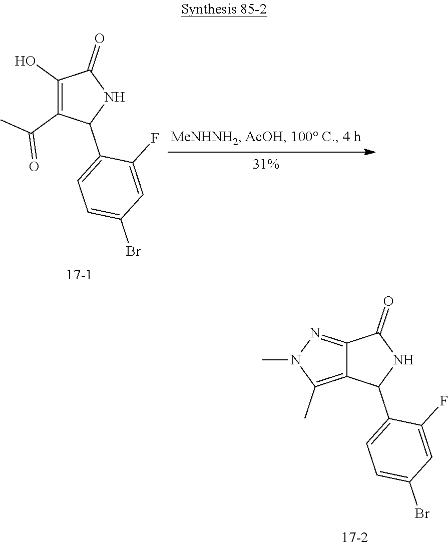 [SMILES: CC(=O)C1=C(O)C(=O)NC1c1ccc(Br)cc1F ; Cc1c2c(nn1C)C(=O)NC2c1ccc(Br)cc1F]

To a solution of 17-1 (1.095 g, 3.5 mmol) in acetic acid (15 mL) was added methylhydrazine (2.0 eq.). The reaction was heated at 100° C. for 4 h. The reaction was quenched with sat. NaHCO₃ and extracted with DCM. The organic layers were dried over sodium sulfate and concentrated in vacuo. The crude product was purified by FCC to give 17-2 as a brown solid (350 mg, 26%). The desired regioisomer elutes first in FCC with 5% MeOH/DCM.

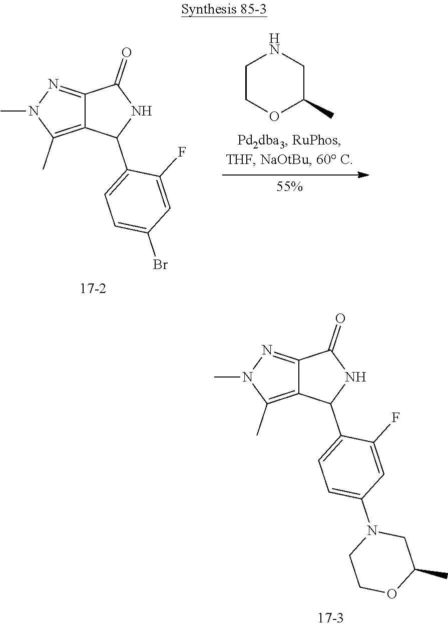

To a solution of 17-2 (32 mg, 0.1 mmol, 1.0 eq) in (1 mL) was added RuPhos (0.5 eq), Pd₂dba₃ (0.25 eq), sodium tert-butoxide (2 eq) and (R)-2-methylmorpholine (3 eq). The reaction was heated at 65° C. for 6 hours. The reaction was quenched with sat. NH₄Cl and extracted with EtOAc. The organic layers were dried over sodium sulfate and concentrated in vacuo. The crude product was purified by FCC to give 17-3 as a white solid (19 mg, 55%).

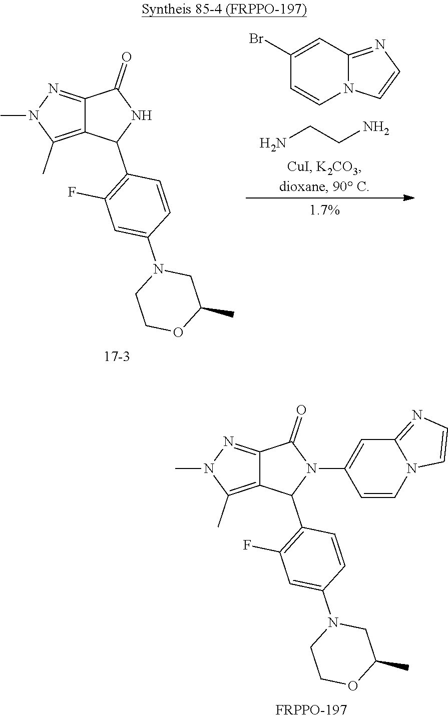

A mixture of 17-3 (112 mg, 0.326 mmol), 7-bromoimidazo[1,2-a]pyridine (128 mg, 2.0 eq.), CuI (310 mg, 5.0 eq.), ethylenediamine (0.2 mL, 10.0 eq.) and potassium carbonate (135 mg, 3.0 eq.) in 1,4-dioxane (2.5 mL) was heated at 90° C. overnight. The reaction was diluted with water and extracted with ethyl acetate. The combined organic layers were dried over sodium sulfate and concentrated in vacuo. The residue purified by silica gel preparative thin layer chromatography, followed by trituration in Et₂O/hexanes to give FRPPO-197 as a white solid (3.0 mg, 1.7%, 97.72% purity).

¹H NMR (400 MHz, Acetonitrile-d3) δ 8.31 (d, J=7.4 Hz, 1H), 7.71 (s, 1H), 7.56 (s, 2H), 7.07 (d, J=9.1 Hz, 1H), 6.61 (d, J=13.7 Hz, 2H), 6.39 (s, 1H), 3.95-3.85 (m, 4H), 3.60 (td, J=11.4, 2.8 Hz, 2H), 3.43 (dd, J=36.4, 12.1 Hz, 2H), 2.71-2.62 (m, 1H), 2.15 (s, 3H), 1.15 (d, J=6.2 Hz, 3H).

LC: Gradient 5% to 95% MeCN:H₂O (0.1% HCOOH), KromegaSil OSD-H, 5.772 min.

LRMS: C₂₅H₂₇F₁NO₂ requires 461.210. Found 460.985.

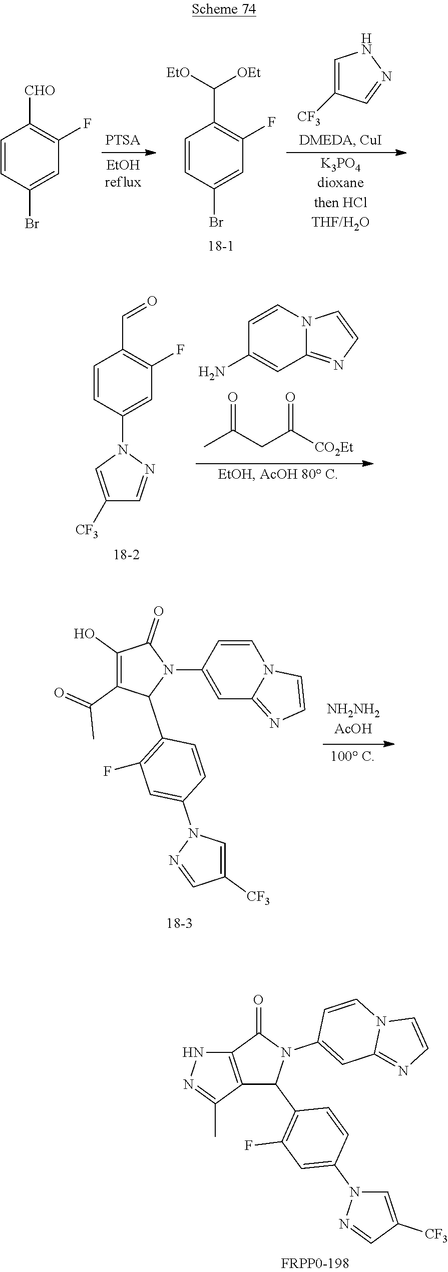

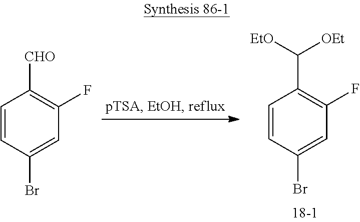 [SMILES: O=Cc1ccc(Br)cc1F ; CCOC(OCC)c1ccc(Br)cc1F]

To a solution of 4-bromo-2-fluorobenzaldehyde (2.03 g, 10 mmol) in EtOH (10 mL) was added pTSA (190 mg, 0.1 eq.) and the reaction was refluxed overnight. The reaction was diluted with sat. NaHCO₃ and extracted with EtOAc. The combined organic layers were dried over sodium sulfate and concentrated in vacuo to give 18-1 as a yellow oil (1.98 g, 7.15 mmol, 72%).

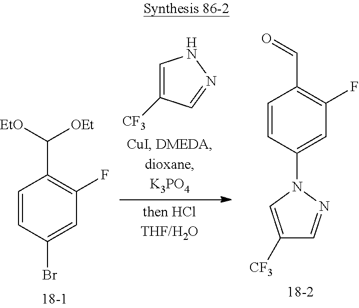 [SMILES: CCOC(OCC)c1ccc(Br)cc1F ; FC(F)(F)c1cn[nH]c1 ; O=Cc1ccc(-n2cc(C(F)(F)F)cn2)cc1F]

A vial was charged with 18-1 (554 mg, 2 mmol), CuI (380 mg, 1eq.), DMEDA (353 mg, 2 eq.), K₃PO₄ (1.70 g, 4 eq.) and dioxane (5 mL). The reaction was flushed with N₂ and heated to 100° C. overnight. The reaction was diluted with sat. NaHCO₃ and extracted with ethyl acetate. The combined organic layers were dried over sodium sulfate and concentrated in vacuo. The reaction was suspended in THF (5 mL) and 1M HCl (5 mL) and stirred for 5 h. The reaction was quenched with NaHCO₃ and extracted with ethyl acetate. The combined organic layers were dried over sodium sulfate and concentrated n vacuo. The crude product was purified by FCC to give 18-2 as a yellow solid, 317 mg, 1.23 mmol, 61%.

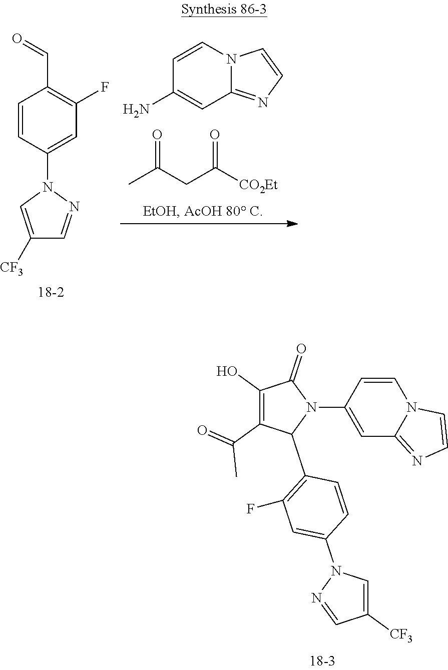

To a solution of 18-2 (300 mg, 1.16 mmol) in EtOH/AcOH (10:1, 5 mL) was added imidazo[1,2-a]pyridin-7-amine (155 mg, 1 eq.) and the reaction heated to 80° C. for 1 h. Next, ethyl 2,4-dioxopentanoate (184 mg, 1 eq.) was added and the reaction heated overnight at 80° C. The reaction was cooled to 0° C. and the crude product collected by filtration to give 18-3 as a red solid, 142 mg, 0.29 mmol, 25%.

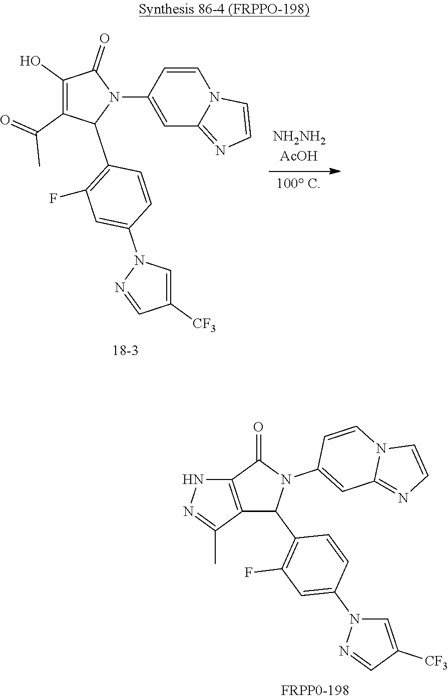 [SMILES: CC(=O)C1=C(O)C(=O)N(c2ccn3ccnc3c2)C1c1ccc(-n2cc(C(F)(F)F)cn2)cc1F ; Cc1n[nH]c2c1C(c1ccc(-n3cc(C(F)(F)F)cn3)cc1F)N(c1ccn3ccnc3c1)C2=O]

To a solution of 18-3 (60 mg, 0.124 mmol) in AcOH (2 mL) was added hydrazine hydrate (2 eq.) and the reaction heated to 100° C. for 2 hrs. The reaction was poured slowly into sat. NaHCO₃ and extracted with ethyl acetate. The combined organic layers were dried over sodium sulfate and concentrated in vacuo. The crude product was purified by FCC followed by preparative reverse phase HPLC to give FRPPO-198 as a tan solid, 1.6 mg, 0.0033 mmol, 2.7%, 97.53% purity.

¹H NMR (400 MHz, Acetonitrile-d3) δ 8.54-8.42 (m, 1H), 8.23 (d, J=7.5 Hz, 1H), 7.95 (s, 1H), 7.64 (s, 1H), 7.60 (dd, J=11.6, 2.2 Hz, 1H), 7.54-7.44 (m, 3H), 7.37 (d, J=8.2 Hz, 1H), 7.33 (d, J=8.6 Hz, 1H), 6.54 (s, 1H), 2.17 (s, 3H).

LC: Gradient 5% to 95% MeCN:H₂O (0.1% HCOOH), KromegaSil OSD-H, 6.405 min.

LRMS: C₂₃H₁₆F₄N₇O₁ requires 482.135. Found 481.982.

### Biological Methods and Data

Initially, isoQC inhibitory activity was measured at a single concentration in a fluorescent-based assay (Assay 1 below). In order to establish the inhibitory activity more quantitatively, IC₅₀ values were obtained using a mass spectrometry-based assay where pyroglutamylation by either isoQC or QC was measured (Assays 2 and 4, respectively, below). Alternatively, IC₅₀ values for isoQC or QC were obtained using a fluorescence-based assay (Assays 3 and 5 respectively, below). Finally, a cell-based assay was deployed to measure the potency of compounds using a combination of an antibody that specifically binds to isoQC dependent pyroglutamylated CD47, as well as an antibody that binds to CD47 independent of its pyroglutamylation status, thereby controlling for inadvertent downregulation of CD47 itself (Assay 6 below).

### Vector Generation

LentiCRISPRv2 vector (Genscript) was cut with PacI and EcoRI restriction enzymes (NEB) for 1 hour at 37° C. (New England Biolabs) in CutSmart buffer (NEB) and the backbone was isolated from gel using a Qiaquick Gel Extraction Kit (Qiagen) according to the manufacturer's protocol.

A synthetic DNA fragment (Sequence ID No. 1) was inserted using NEBuilder HiFi DNA Assembly Master Mix (New England Biolabs) and ensuing GIBSON assembly for 30 minutes at 50° C. and transformation into Endura bacteria (Lucigen) to obtain pSCNC-LentiCRISPR.

Sequence ID No. 1
CAGGGACAGC AGAGATCCAG TTTGGTTAAT TAAGGTACCG
AGGGCCTATT TCCCATGATT CCTTCATATT TGCATATACG
ATACAAGGCT GTTAGAGAGA TAATTAGAAT TAATTTGACT
GTAAACACAA AGATATTAGT ACAAAATACG TGACGTAGAA
AGTAATAATT TCTTGGGTAG TTTGCAGTTT TAAAATTATG
TTTTAAAATG GACTATCATA TGCTTACCGT AACTTGAAAG
TATTTCGATT TCTTGGCTTT ATATATCTTG TGGAAAGGAC
GAAACACCGG AGACGGATTA ATTAAACCGT CTCAGTTTAA
GAGCTAGAAA TAGCAAGTTT AAATAAGGCT AGTCCGTTAT
CAACTTGAAA AAGTGGCACC GAGTCGGTGC TTTTTTGAAT
TCGCTAGCTA GGTCTTGAAA GGAGTGG (IDTDNA)

Subsequently, BsmBI (New England Biolabs) digested pSCNC-LentiCRISPR (1 hour at 55° C. in buffer 3.1 (NEB)) was isolated from gel using a Qiaquick Gel Extraction Kit (Qiagen) and Oligonucleotides encoding gRNAs targeting QPCTL (Sequence ID No. 2) or CD47 (Sequence ID No. 3) were cloned in using a 30 minute GIBSON assembly at 50° C. using NEBuilder HiFi DNA Assembly Master Mix (New England Biolabs) and subsequent transformation into Endura cells to obtain pSCNC-LentiCRISPR-QPCTL and pSCNC-LentiCRISPR-CD47 respectively.

Sequence ID No. 2
TATCTTGTGG AAAGGACGAA ACACCGCGGG GAGGCTTCCG
ATCAATGTTT AAGAGCTAGA AATAGCAAGT TTAAA
Sequence ID No. 3
TATCTTGTGG AAAGGACGAA ACACCGCTAC TGAAGTATAC
GTAAAGGTTT AAGAGCTAGA AATAGCAAGT TTAAA

### Total CD47/pE-CD47 Dual Staining

Cells were spun for 4 minutes at 1500 rpm and washed once with PBS/0.5% BSA. Cells were spun down again and the supernatant was removed and incubated in 100 uL antibody staining solution (PBS/0.5% BSA/0.1 μg/mL DAPI (4′,6-diamidino-2-phenylindole, ThermoFisher Scientific) and a mixture of 1:500 FITC-conjugated anti-human CD47 clone 2D3, which recognizes total CD47 irrespective of its pyroglutamylation state, and 1:500 Alexa647-conjugated anti-human CD47 clone CC2C6, which specifically detects pyroglutamylated CD47) for 2 hours at 4° C. Next, the cells were spun down and washed twice with 2×150 μL PBS/0.5% BSA to remove unbound antibody. Cells were again spun down and taken up in 300 μL FACS buffer before analysis on a Celesta FACS machine (BD Biosciences).

### Creation of QPCTL and CD47 Knockout Cells

KBM7 cells (Kotecki et al., 1999) were infected with lentivirus produced by transfecting pSCNC-LentiCRISPR-QPCTL or pSCNC-LentiCRISPR-CD47 into HEK-293T cells according to standard protocols. After 24 hours incubation, cells were selected with puromycin (Invivogen, 1 μg/mL) for 48 hours. Cells were stained for total CD47 and pE-CD47 and QPCTL k.o. cells were sorted out as negative for pE-CD47, and CD47 k.o. cells as negative for total CD47, using a FACSAria III and FACS Diva software (BD Biosciences).

Recombinant isoQC Production

The Golgi luminal, enzymatically active region of human isoQC (S53-L382; see, e.g., Huang et al., 2011) was obtained by expression in E. coli of an N-terminally GST-Enterokinase and C-terminally 6×His tagged construct in the pET41(+) vector and subsequent purification using talon beads and GST beads, enterokinase digestion and further purification with a final formulation in 25 mM Tris pH 8.0/150 mM NaCl/1 mM TCEP. Absence of pyroglutamylating activity in an enzymatic dead variant that was cloned, expressed, and purified in parallel ruled out that the purification strategy isolates any endogenously present pyroglutamylating activity.

Assay 1—isoQC Inhibition—Fluorescent Enzymatic Assay

Pyroglutamylation was measured by conversion of glutamine-4-amino-7-methylcoumarine (H-Gln-AMC, Bachem) to pyroglutamyl-AMC, which is subsequently a specific substrate for pyroglutamylaminopeptidase (pGAPase, Qiagen), releasing free AMC which can be fluorescently detected. For a typical inhibition assay, 15 μL 20 μM h-Gln-AMC (diluted from a 40 mM stock dissolved in 25 mM HEPES using 50 mM Tris-HCl pH 8.0) was pipetted into a 96-well plate on ice. Next, 7.5 μL 1 μM test compound (diluted from a 10 mM stock dissolved in DMSO using 50 mM Tris-HCl) was added. Finally, 7.5 μL, 3 ng/μL recombinant isoQC enzyme was added and the reaction mixture was incubated for 1 hour at 37° C. A 5 minute incubation at 98° C. was then performed to denature the isoQC enzyme and stop the reaction.

To detect the amount of pyroglutamyl-AMC formed, a 96-well plate was prepared with 25 μL 0.125 U/mL pGAPase enzyme diluted in 50 mM Tris-HCl pH 8.0 containing 10 mM dithiothreitol (DTT). 25 μL of the pyroglutamylation reaction mixture was transferred into the plate containing the pGAPase solutions while on ice. Once done, the plate was moved over to a Spectramax ID3 fluorescent plate-reader to monitor and record the maximum fluorescence at 380 nm excitation/450 nm emission over a period of 20 minutes while keeping the temperature at 37° C.

DMSO concentrations during the assay never exceeded 1% and activities were compared to controls containing only the same amount of DMSO and no test compound. Controls were taken along containing pyroglutamylated-AMC (Bachem, dissolved at 10 mM in DMSO) instead of H-Gln-AMC to control for pGAPase inhibition but no test compound that was tested showed such activity. Test compounds were reported as active when <40% of background corrected signal was measured as compared to DMSO controls.

Assay 2—isoQC Potency—Mass Spectrometry-Based Enzymatic Potency Assay

EC₅₀'s of test compounds were determined by incubating 11-point two-fold dilutions of test compound in 20 μL volumes in the presence of 7.5 nM isoQC and 10 μM H-Gln-AMC using 50 mM Tris-HCl pH 8.0 as assay buffer for 2 hours at room temperature before stopping the reaction by the addition of 2 volumes of methanol. Both the substrate and the product of this reaction were read out using an Agilent RapidFire microfluidic solid phase sample extraction system coupled to an AB Sciex API 4000 triple quadrupole mass spectrometer, in negative ion mode, at m/z of 301.9 and 284.9 respectively. The actual readout was defined as P/(S+P), where P is the product signal area and S the substrate signal area. EC₅₀'s were calculated by four parameter sigmoidal model fitting.

Assay 3—isoQC Potency—Fluorescent Enzymatic Assay

For this assay specifically, isoQC production was performed slightly different than described in the general methods: The Golgi luminal, enzymatically active region of human isoQC (S53-L382; see, e.g., Huang et al., 2011) was obtained by expression in E. coli of an N-terminally GST Enterokinase and C-terminally 6×His tagged construct in the pET41(+) vector and subsequent purification using Ni-NTA IMAC. Enterokinase digestion ensued, followed by another Ni-NTA IMAC purification, Superdex75 based size exclusion and finally a spin concentration using a 10 kDa molecular weight cutoff. The final protein is formulated in 50 mM Tris-HCl, 150 mM NaCl, 50% Glycerol, pH 7.8.

Pyroglutamylation was measured by conversion of glutamine-4-amino-7-methylcoumarine (H-Gln-AMC, Bachem) to pyroglutamyl-AMC, which is subsequently a specific substrate for pyroglutamylaminopeptidase (pGAPase, Qiagen), releasing free AMC which can be fluorescently detected. For a typical inhibition assay, 7.5 μL aliquots of threefold dilutions of test compound in 50 mM Tris pH8 buffer were plated in 384-well plates. 15 μL 20 μM h-Gln-AMC (diluted from a 40 mM stock dissolved in 25 mM HEPES using 50 mM Tris-HCl pH 8.0) was added to each well. Finally, 7.5 μL, 3 ng/μL recombinant isoQC enzyme was added and the reaction mixture was incubated for 1 hour at 37° C. A 5 minute incubation at 98° C. was then performed to denature the isoQC enzyme and stop the reaction.

To detect the amount of pyroglutamyl-AMC formed, a 0.125 U/mL pGAPase enzyme diluted in 50 mM Tris-HCl pH 8.0 containing 10 mM dithiothreitol (DTT) solution was prepared. 25 μL of the pyroglutamylation reaction mixture was transferred into an empty plate and to that, 25 μL of the pGAPase enzyme solution was added. Plates were then incubated for 25 minutes at room temperature before fluorescence readings were taken on an Envision2 plate reader with excitation set at 380 nm and emission at 450 nm.

DMSO concentrations during the assay never exceeded 1% and activities were compared to controls containing only the same amount of DMSO and no test compound. Controls were taken along containing pyroglutamylated-AMC (Bachem, dissolved at 10 mM in DMSO) instead of H-Gln-AMC to control for pGAPase inhibition. IC₅₀ values are calculated using non-linear regression analysis with GraphPad Prism software.

### Assay 4—QC Potency—Mass Spectrometry-Based Enzymatic Potency Assay

EC₅₀'s of test compounds were determined in the same way as described in Assay 2 above, except that N-terminally His6-tagged recombinant human QC (R&D Systems) was used instead of isoQC.

### Assay 5—QC Potency—Fluorescent Enzymatic Assay

Recombinant QC enzyme was produced by bacterial expression of residues 33-361 of uniprot ID Q16769, fused to a His tag, codon optimized and cloned into the pET32a plasmid backbone. After scale-up, expression and lysis, protein was purified by Ni-NTA IMAC, dialysed in the presence of Factor Xa, further purified by Ni-NTA reverse IMAC and finally filtrated for size on a Superdex 200 16/60 column. Final protein was concentrated using a spin concentrator at 10 kDa molecular weight cutoff and formulated in 150 mM NaCl, 50 mM Tris-HCl, 50% Glycerol, pH 8.0.

Pyroglutamylation was measured by conversion of glutamine-4-amino-7-methylcoumarine (H-Gln-AMC, Bachem) to pyroglutamyl-AMC, which is subsequently a specific substrate for pyroglutamylaminopeptidase (pGAPase, Qiagen), releasing free AMC which can be fluorescently detected. For a typical inhibition assay, 7.5 μL aliquots of threefold dilutions of test compound in 50 mM Tris pH8 buffer were plated in 384-well plates. 15 μL 20 μM h-Gln-AMC (diluted from a 40 mM stock dissolved in 25 mM HEPES using 50 mM Tris-HCl pH 8.0) was added to each well. Finally, 7.5 μL, 3 ng/μL recombinant QC enzyme was added and the reaction mixture was incubated for 1 hour at 37° C. A 5 minute incubation at 98° C. was then performed to denature the QC enzyme and stop the reaction.

To detect the amount of pyroglutamyl-AMC formed, a 0.125 U/mL pGAPase enzyme diluted in 50 mM Tris-HCl pH 8.0 containing 10 mM dithiothreitol (DTT) solution was prepared. 25 μL of the pyroglutamylation reaction mixture was transferred into an empty plate and to that, 25 μL of the pGAPase enzyme solution was added. Plates were then incubated for 25 minutes at room temperature before fluorescence readings were taken on an Envision2 plate reader with excitation set at 380 nm and emission at 450 nm.

DMSO concentrations during the assay never exceeded 1% and activities were compared to controls containing only the same amount of DMSO and no test compound. Controls were taken along containing pyroglutamylated-AMC (Bachem, dissolved at 10 mM in DMSO) instead of H-Gln-AMC to control for pGAPase inhibition.

IC₅₀ values are calculated using non-linear regression analysis with GraphPad Prism software.

### Assay 6—Cell-Based Potency Assay

In order to test compounds for their effect on pyroglutamylation in live cells, 25000 KBM7 cells per well were seeded in a 96-well U-bottom plate in 150 μl IMDM/10% FCS/1% Pen/Strep medium and a 10-point 2-fold dilution range of test compounds. After 72 hours incubation at 37° C., 5% CO₂, cells were stained for total CD47 and pE-CD47 and analyzed on a BD Celesta using FACS Diva software (BD Biosciences). Cells gated for DAPI negativity were recorded and the median fluorescence intensity (MFI) of each sample was transformed to linearly scale between the MFI's of wildtype and CD47 knockout cells (in case of the 2D3 antibody) and the wildtype and QPCTL knockout cells (in case of the CC2C6 antibody). IC₅₀'s were calculated by 3 parameter logistic regression of the CC2C6 signal vs. compound concentration, where the top of the sigmoidal curve was fixed at 1, using R and the drc package (http://www.r-project.org, https://cran.r-project.org/web/packages/drc/index.html).

The biological data for Assays 1-6 are summarized in the following table.

TABLE 1
Biological Data - Assays 1 to 6

| | | Basis | | | | | |
|---|---|---|---|---|---|---|---|
| | | isoQc | isoQc | isoQc | QC | QC | Cell |
| | | Type | | | | | |
| | | Inh. | Pot. | Pot. | Pot. | Pot. | Pot. |
| | | Assay No. | | | | | |
| Code | Notes | 1 | 2 | 3 | 4 | 5 | 6 |
| FRPPO-001 | Racemate | | | | | | +++ |
| FRPPO-001A | Peak 1 | − | | | | | (§) |
| FRPPO-001B | Peak 2 | + | +++ | | ++ | | +++ |
| FRPPO-002 | Racemate | | | | | | ++ |
| FRPPO-002A | Peak 1 | − | | | | | ++ |
| FRPPO-002B | Peak 2 | − | | | | | (§§) |
| FRPPO-003 | Racemate | + | ++ | | + | | +++ |
| FRPPO-004 | Racemate | + | +++ | | ++ | | +++ |
| FRPPO-005A | Racemate | + | +++ | | ++ | | +++ |
| FRPPO-005B | Peak 2 | | | | | | +++ |
| FRPPO-006 | Racemate | + | ++ | | + | | +++ |
| FRPPO-007 | Racemate | + | ++ | | ++ | | +++ |
| FRPPO-008A | Racemate | + | ++ | | ++ | | +++ |
| FRPPO-008B | Peak 1 | | | | | | +++ |
| FRPPO-009 | Racemate | + | ++ | | + | | ++ |
| FRPPO-010 | Racemate | + | ++ | | ++ | | ++ |
| FRPPO-011 | Racemate | + | ++ | | + | | − |
| FRPPO-012 | Racemate | + | +++ | | ++ | | +++ |
| FRPPO-013 | Racemate | + | ++ | | ++ | | ++ |
| FRPPO-014 | Racemate | + | +++ | | ++ | | ++ |
| FRPPO-015A | Racemate | + | +++ | | ++ | | +++ |
| FRPPO-015B | Peak 2 | | | | | | +++ |
| FRPPO-016 | Racemate | + | +++ | | ++ | | + |
| FRPPO-017 | Racemate | + | ++ | | ++ | | − |
| FRPPO-018 | Racemate | + | +++ | | +++ | | + |
| FRPPO-019 | Racemate | + | ++ | | +++ | | +++ |
| FRPPO-020 | Racemate | + | +++ | | ++ | | ++ |
| FRPPO-021 | Racemate | − | | | | | + |
| FRPPO-022 | Racemate | − | | | | | ++ |
| FRPPO-023 | Racemate | − | | | | | + |
| FRPPO-025 | Racemate | + | +++ | | ++ | | + |
| FRPPO-026A | Racemate | + | +++ | | +++ | | +++ |
| FRPPO-026B | Peak 2 | | | | | | +++ |
| FRPPO-027 | Racemate | + | +++ | | +++ | | ++ |
| FRPPO-028A | Racemate | + | +++ | | ++ | | +++ |
| FRPPO-028B | Peak 1 | − | | | | | ++ |
| FRPPO-028C | Peak 2 | + | | | | | +++ |
| FRPPO-029 | Racemate | + | ++ | | + | | +++ |
| FRPPO-030 | Racemate | + | + | | + | | ++ |
| FRPPO-031 | Racemate | + | ++ | | + | | ++ |
| FRPPO-032 | Racemate | − | + | | + | | ++ |
| FRPPO-033 | Racemate | + | + | | + | | ++ |
| FRPPO-034 | Racemate | + | + | | + | | ++ |
| FRPPO-035 | Racemate | + | + | | + | | ++ |
| FRPPO-036 | Racemate | + | | | | | +++ |
| FRPPO-037 | Racemate | + | | | | | ++ |
| FRPPO-038 | Racemate | + | | | | | ++ |
| FRPPO-039A | Racemate | | | | | | +++ |
| FRPPO-039B | Peak 1 | | | | | | ++ |
| FRPPO-039C | Peak 2 | | | | | | +++ |
| FRPPO-040 | Racemate | | | | | | +++ |
| FRPPO-046 | Racemate | | | | | | + |
| FRPPO-047 | Racemate | | | | | | + |
| FRPPO-048A | Racemate | | | | | | +++ |
| FRPPO-048B | Peak 2 | | | | | | +++ |
| FRPPO-049 | Racemate | | | | | | +++ |
| FRPPO-050 | Racemate | | | | | | ++ |
| FRPPO-051 | Racemate | | | | | | ++ |
| FRPPO-052 | Racemate | | | | | | + |
| FRPPO-053 | Racemate | | | | | | + |
| FRPPO-054 | Racemate | | | | | | ++ |
| FRPPO-057 | Racemate | | | | | | + |
| FRPPO-058 | Racemate | | | | | | + |
| FRPPO-061 | Racemate | | | | | | ++ |
| FRPPO-063 | Racemate | | | | | | ++ |
| FRPPO-064 | Racemate | | | | | | + |
| FRPPO-066 | Racemate | | | | | | ++ |
| FRPPO-067A | Racemate | | | | | | +++ |
| FRPPO-067B | Peak 2 | | | | | | +++ |
| FRPPO-068A | Racemate | | | | | | +++ |
| FRPPO-068B | Peak 2 | | | | | | +++ |
| FRPPO-069 | Racemate | | | | | | ++ |
| FRPPO-070 | Racemate | | | | | | ++ |
| FRPPO-072 | Racemate | | | | | | +++ |
| FRPPO-073 | Racemate | | | | | | ++ |
| FRPPO-074A | Racemate | | | | | | +++ |
| FRPPO-074B | Peak 2 | | | | | | +++ |
| FRPPO-077 | Racemate | | | | | | ++ |
| FRPPO-078 | Racemate | | | | | | +++ |
| FRPPO-079 | Racemate | | | | | | ++ |
| FRPPO-081 | Racemate | | | | | | ++ |
| FRPPO-082 | Racemate | | | | | | + |
| FRPPO-083 | Racemate | | | | | | ++ |
| FRPPO-084A | Racemate | | | | | | +++ |
| FRPPO-084B | Peak 1 | | | | | | ++ |
| FRPPO-084C | Peak 2 | | | | | | +++ |
| FRPPO-084D | Peak 3 | | | | | | +++ |
| FRPPO-084E | Peak 4 | | | | | | +++ |
| FRPPO-085 | Racemate | | | | | | + |
| FRPPO-086 | Racemate | | | | | | ++ |
| FRPPO-087A | Racemate | | | | | | +++ |
| FRPPO-087B | Peak 2 | | | | | | +++ |
| FRPPO-088A | Racemate | | | | | | +++ |
| FRPPO-088B | Peak 2 | | | | | | +++ |
| FRPPO-089 | Racemate | | | | | | ++ |
| FRPPO-090A | Racemate | | | | | | +++ |
| FRPPO-090B | Peak 1 | | | | | | ++ |
| FRPPO-090C | Peak 2 | | | | | | +++ |
| FRPPO-091A | Racemate | | | | | | +++ |
| FRPPO-091B | Peak 2 | | | | | | +++ |
| FRPPO-092 | Racemate | | | | | | +++ |
| FRPPO-093 | Racemate | | | | | | ++ |
| FRPPO-094A | Racemate | | | | | | +++ |
| FRPPO-094B | Peak 2 | | | | | | +++ |
| FRPPO-095 | Racemate | | | | | | ++ |
| FRPPO-096 | Racemate | | | | | | +++ |
| FRPPO-097 | Racemate | | | | | | +++ |
| FRPPO-098A | Racemate | | | | | | +++ |
| FRPPO-098B | Peak 2 | | | | | | ++ |
| FRPPO-100 | Racemate | | | | | | + |
| FRPPO-101A | Racemate | | | | | | +++ |
| FRPPO-101B | Peak 1 | | | | | | ++ |
| FRPPO-101C | Peak 2 | | | | | | +++ |
| FRPPO-102A | Racemate | | | | | | +++ |
| FRPPO-102B | Peak 2 | | | | | | +++ |
| FRPPO-103A | Racemate | | | | | | +++ |
| FRPPO-103B | Peak 2 | | | | | | +++ |
| FRPPO-104 | Racemate | | | | | | +++ |
| FRPPO-105 | Racemate | | | | | | + |
| FRPPO-106A | Racemate | | | | | | +++ |
| FRPPO-106B | Peak 2 | | | | | | +++ |
| FRPPO-107 | Racemate | | | | | | ++ |
| FRPPO-108A | Racemate | | | | | | +++ |
| FRPPO-108B | Peak 1 | | | | | | +++ |
| FRPPO-108C | Peak 2 | | | | | | +++ |
| FRPPO-109 | Racemate | | | | | | +++ |
| FRPPO-110A | Racemate | | | | | | +++ |
| FRPPO-110B | Peak 2 | | | | | | +++ |
| FRPPO-111 | Racemate | | | | | | ++ |
| FRPPO-112A | Racemate | | | | | | +++ |
| FRPPO-112B | Peak 2 | | | | | | +++ |
| FRPPO-113A | Mixture | | | | | | +++ |
| FRPPO-113B | Mixture | | | | | | +++ |
| FRPPO-114A | Mixture | | | | | | +++ |
| FRPPO-114B-A | Mixture | | | | | | +++ |
| FRPPO-114B-B | Peak 2 | | | | | | +++ |
| FRPPO-115A | Mixture | | | | | | +++ |
| FRPPO-115B | Mixture | | | | | | ++ |
| FRPPO-126B | Peak 1 | | | | | | ++ |
| FRPPO-126C | Peak 2 | | | | | | +++ |
| FRPPO-127B | Peak 2 | | | | | | +++ |
| FRPPO-134A | Racemate | | | | | | +++ |
| FRPPO-134B | Peak 2 | | | | | | +++ |
| FRPPO-135A | Racemate | | | | | | +++ |
| FRPPO-135B | Peak 2 | | | | | | +++ |
| FRPPO-136 | Racemate | | | | | | +++ |
| FRPPO-137 | Racemate | | | | | | +++ |
| FRPPO-142A | Racemate | | | | | | +++ |
| FRPPO-142B | Peak 2 | | | | | | +++ |
| FRPPO-143A | Racemate | | | | | | +++ |
| FRPPO-143B | Peak 1 | | | | | | +++ |
| FRPPO-144 | Racemate | | | | | | +++ |
| FRPPO-145 | Racemate | | | | | | +++ |
| FRPPO-146 | Racemate | | | | | | +++ |
| FRPPO-147 | Racemate | | | | | | +++ |
| FRPPO-148A | Racemate | | | | | | +++ |
| FRPPO-148B | Peak 2 | | | | | | +++ |
| FRPPO-149A | Racemate | | | | | | +++ |
| FRPPO-149B | Peak 1 | | | | | | +++ |
| FRPPO-150A | Racemate | | | | | | +++ |
| FRPPO-150B | Peak 2 | | | | | | +++ |
| FRPPO-151A | Racemate | | | | | | +++ |
| FRPPO-151B | Peak 1 | | | | | | +++ |
| FRPPO-152A | Racemate | | | | | | +++ |
| FRPPO-152B | Peak 1 | | | | | | +++ |
| FRPPO-153 | Racemate | | | | | | +++ |
| FRPPO-154 | Racemate | | | | | | +++ |
| FRPPO-155 | Racemate | | | | | | + |
| FRPPO-156 | Mixture | | | | | | +++ |
| FRPPO-157 | Racemate | | | | | | +++ |
| FRPPO-158A | Racemate | | | | | | +++ |
| FRPPO-158B | Peak 1 | | | + | | + | |
| FRPPO-158C | Peak 2 | | | ++ | | ++ | |
| FRPPO-159A | Racemate | | | | | | +++ |
| FRPPO-159B | Peak 1 | | | + | | + | |
| FRPPO-159C | Peak 2 | | | +++ | | + | |
| FRPPO-160B | Peak 1 | | | + | | − | + |
| FRPPO-160C | Peak 2 | | | +++ | | +++ | +++ |
| FRPPO-161 | Racemate | | | ++ | | ++ | +++ |
| FRPPO-162B | Peak 1 | | | + | | + | |
| FRPPO-162C | Peak 2 | | | +++ | | ++ | +++ |
| FRPPO-164 | Racemate | | | + | | + | +++ |
| FRPPO-165 | Racemate | | | ++ | | + | +++ |
| FRPPO-166 | Racemate | | | + | | + | ++ |
| FRPPO-167 | Racemate | | | | | | ++ |
| FRPPO-168 | Racemate | | | | | | ++ |
| FRPPO-169 | Racemate | | | | | | ++ |
| FRPPO-170 | Racemate | | | | | | ++ |
| FRPPO-171 | Racemate | | | + | | + | |
| FRPPO-173 | Racemate | | | ++ | | ++ | |
| FRPPO-174 | Racemate | | | | | | ++ |
| FRPPO-175 | Racemate | | | | | | +++ |
| FRPPO-176 | Racemate | | | ++ | | + | |
| FRPPO-177 | Racemate | | | +++ | | ++ | |
| FRPPO-197 | Racemate | | | | | | +++ |
| FRPPO-198 | Racemate | | | | | | +++ |

Key:
For Assay 1 (isoQC Inhibition):
(−) ≤ 40% isoQC inhibition at 250 nM; (+) > 40% isoQC inhibition at 250 nM.
For Assay 2, Assay 3, Assay 4, and Assay 5 (isoQC/QC Potency):
(−) IC₅₀ ≥ 5 μM; (+) 100 nM < IC₅₀ < 5 μM; (++) 25 nM < IC₅₀ < 100 nM; (+++) IC₅₀ < 25 nM.
For Assay 6 (Cell-based Potency):
(−) IC₅₀ ≥ 50 μM; (+) 5 μM < IC₅₀ < 50 μM; (++) 1 μM < IC₅₀ < 5 μM; (+++) IC₅₀ < 1 μM.
(§) Tested up to 15 μM, no IC₅₀ reached.
(§§) Tested up to 3 μM, no IC₅₀ reached.

### Comparison Data

In addition, the following reference compounds were prepared for the purpose of comparing cell potency of reference compounds with the FRPPO compounds described herein. Each reference compound differs from FRPPO-001 only by the group -Q.

TABLE 2
Biological Data - Comparison Data

| | | | Cell |
|---|---|---|---|
| | | | Po- |
| | | | tency |
| | | | Assay |
| Code | Chemical Structure | Notes | 6 |
| FRPPO- 001 | 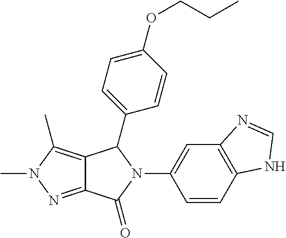 [SMILES: CCCOc1ccc(C2c3c(nn(C)c3C)C(=O)N2c2ccc3[nH]cnc3c2)cc1] | Race- mate | +++ |
| REF-001 | 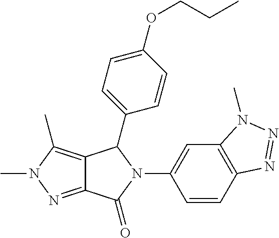 [SMILES: CCCOc1ccc(C2c3c(nn(C)c3C)C(=O)N2c2ccc3nnn(C)c3c2)cc1] | Race- mate | (§§) |
| REF-002 | 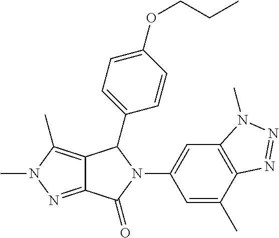 [SMILES: CCCOc1ccc(C2c3c(nn(C)c3C)C(=O)N2c2cc(C)c3nnn(C)c3c2)cc1] | Race- mate | (§§) |
| REF-003 | 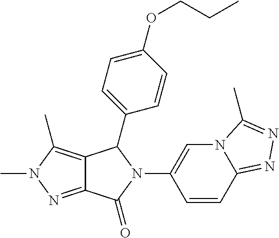 [SMILES: CCCOc1ccc(C2c3c(nn(C)c3C)C(=O)N2c2ccc3nnc(C)n3c2)cc1] | Race- mate | — |
| REF-004 | 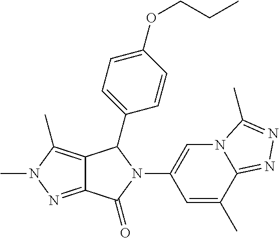 [SMILES: CCCOc1ccc(C2c3c(nn(C)c3C)C(=O)N2c2cc(C)c3nnc(C)n3c2)cc1] | Race- mate | — |
| REF-005 | 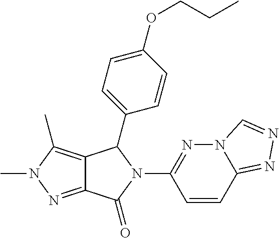 [SMILES: CCCOc1ccc(C2c3c(nn(C)c3C)C(=O)N2c2ccc3nncn3n2)cc1] | Race- mate | — |
| REF-006 | 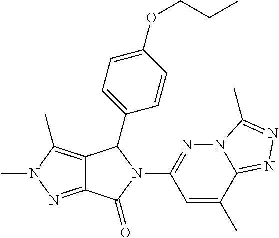 [SMILES: CCCOc1ccc(C2c3c(nn(C)c3C)C(=O)N2c2cc(C)c3nnc(C)n3n2)cc1] | Race- mate | (§§) |
| REF-007 | 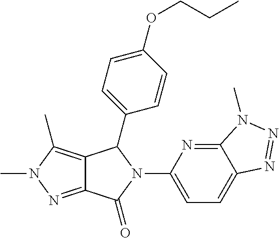 [SMILES: CCCOc1ccc(C2c3c(nn(C)c3C)C(=O)N2c2ccc3nnn(C)c3n2)cc1] | Race- mate | (§§) |
| REF-008 | 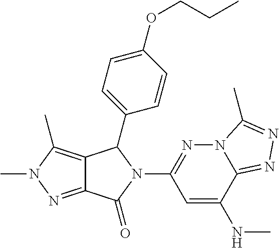 [SMILES: CCCOc1ccc(C2c3c(nn(C)c3C)C(=O)N2c2cc(NC)c3nnc(C)n3n2)cc1] | Race- mate | — |

Key:
(—) IC₅₀ ≥ 50 μM; (+) 5 μM < IC₅₀ < 5 μM; (++) 1 μM < IC₅₀ < 5 μM; (+++) IC₅₀ < 1 μM.
(§) Tested up to 15 μM, no IC₅₀ reached.
(§§) Tested up to 6.25 μM, no IC₅₀ reached.

### Additional Biological Methods and Data

### Assay 7—CACO-2-Based Cell-Permeation and Efflux—Protocol W

CACO-2 (ATCC, Manassas, Va., USA) monolayers on polyethylene membranes in 96-well insert plates were incubated for 2 hours in 5% CO₂, 37° C. with 2 μM compound tested bi-directionally in duplicate using HBSS with 10 mM HEPES at pH 7.40±0.05 as transport buffer. Final DMSO Concentrations were adjusted to less than 1%. All samples were mixed with acetonitrile containing internal standard and centrifuged at 3220×g for 10 minutes. Subsequently, 100 μL supernatant solution was diluted with 100 μL distilled water for LC/MS/MS analysis. Lucifer yellow rejection assays were applied after the transport assay to ensure CACO-2 cell monolayer integrity.

The apparent permeability coefficient Papp (cm/s) was calculated using the equation:

Papp=(dCr/dt)×Vr/(A×C0)

where dCr/dt is the cumulative concentration of compound in the receiver chamber as a function of time (μM/s); Vr is the solution volume in the receiver chamber (0.075 mL on the apical side, 0.25 mL on the basolateral side); A is the surface area for the transport, i.e., 0.0804 cm² for the area of the monolayer; C0 is the initial concentration in the donor chamber (μM).

28 Compounds were tested and several were found to have very suitable apparent apical to basal permeation rates.

### Assay 8—CACO-2-Based Cell-Permeation and Efflux—Protocol C

CACO-2 monolayers in 96-well insert plates were incubated for 2 hours in 5% CO₂, 37° C. with 10 μM compound tested bi-directionally in duplicate using HBSS with 10 mM HEPES at pH 7.40±0.05 as transport buffer. Final DMSO Concentrations were adjusted to 1%. All samples were mixed with internal standards and sent for LC/MS/MS analysis. Lucifer yellow was included in the assays to ensure CACO-2 cell monolayer integrity.

The apparent permeability coefficient Papp (cm/s) was calculated using the equation:

Papp=(dCr/dt)×Vr/(A×C0)

where dCr/dt is the cumulative concentration of compound in the receiver chamber as a function of time (μM/s); Vr is the solution volume in the receiver chamber (0.09 mL on the apical side, 0.21 mL on the basolateral side); A is the surface area for the transport, i.e., 0.11 cm² for the area of the monolayer; C0 is the initial concentration in the donor chamber (μM).

13 compounds were tested and several were found to have very suitable apparent apical to basal permeation rates.

### Assay 9—Mouse Microsomal Stability

In order to assess metabolic stability of test compounds, commercially acquired CD-1 mouse liver microsome preparations were incubated in 100 μL at a final concentration of 0.5 mg protein/mL, 1 μM test compound, 0.99% methanol and 0.01% DMSO, after a 10 minute pre-incubation at 37° C. and addition of 0.1 volumes 10 U/mL NADPH in 10 mM MgCl₂. Reactions were stopped by addition of 300 μL cold acetonitrile containing internal standards at t=0, 5, 10, 20, 30, and 60 minutes. Samples were submitted to LC-MS/MS analysis subsequently and the slope of linear regression of time versus ln(% Remaining concentration) was derived to calculate t½ as t½=ln(2)/(−Slope). Then Cl_{int(mic)} and Cl_{int(Liver)} were calculated as follows:

$${CL}_{int ( mic )} = \frac{0.693}{In vitro {T}_{1 / 2}} \cdot \frac{1}{mg / mL microsomal protein in reaction system}$$

$${CL}_{int ( liver )} = {CL}_{int ( mic )} \cdot \frac{mg microsomes}{g liver} \cdot \frac{g liver}{kg body weight}$$

where 45 mg microsomal protein/g liver and 88 g liver/kg mouse bodyweight were used as standardized parameters.

49 compounds were tested and several were found to have very suitable Cₗᵢₙₜ₍ₗᵢᵥₑᵣ₎ values.

### Assay 10—Low Dose Pharmacokinetics

In order to determine the stability of test compounds in plasma, low-dose pharmacokinetic experiments were carried out in C57Bl/6 mice. Test compounds were dissolved at 0.1 mg/mL in an appropriate vehicle and dosed intraperitoneally at 1 mg per kg bodyweight in three animals per compound. At various (between 5 and 9) timepoints after injection, up to 24 hours, blood samples were drawn, plasma isolated and test compound concentrations were determined using LC-MS/MS. t½ values were subsequently calculated using Phoenix WinNonlin 6.3 software and averaged over the three mice per test compound.

The plasma half-life of 29 representative compounds injected at a low dose of 1 mg per kg bodyweight ranged from 0.53 to 2.01 hours.

### Assay 11—Pharmacodynamics

In vivo effects were determined by dosing animals either with test compound or its vehicle at 50 mg/kg intraperitoneally every 12 hours for 7 days. Twelve hours after the last dose was given, animals were euthanized and peripheral blood mononuclear cells (PBMCs) were isolated and cryopreserved into aliquots. Cells were then incubated with 5 μg/mL biotinylated recombinant SIRPα protein (Acro Biosystems) for 2 hours at 4° C., washed, stained with 4 μg/mL Streptavidin-Alexa Fluor 647 and 1:200 Zombie Aqua life/dead stain for 1 hour at 4° C. Cells were again washed and analyzed using a BD Celesta flow cytometer. Live lymphocytes were gated based on forward and side scatter as well as Zombie staining and median levels of SIRPα binding were determined.

For two representative compounds, a reduction of 51% and 22%, respectively, in median SIRPα binding signal was observed.

The foregoing has described the principles, preferred embodiments, and modes of operation of the present invention. However, the invention should not be construed as limited to the particular embodiments discussed. Instead, the above-described embodiments should be regarded as illustrative rather than restrictive. It should be appreciated that variations may be made in those embodiments by workers skilled in the art without departing from the scope of the present invention.

### REFERENCES

Publications are cited herein in order to more fully describe the state of the art to which the invention pertains. Full citations for these references are provided below.

Each of these references is incorporated herein by reference in its entirety into the present disclosure, to the same extent as if each individual reference was specifically and individually indicated to be incorporated by reference.
Batista et al., 1996, “The two membrane isoforms of human IgE assemble into functionally distinct B cell antigen receptors”, J. Exp. Med., Vol. 184, No. 6, pp. 2197-2205.
Blank et al., 2014a, “Imidazopyrrolidinone derivatives and their use in the treatment of disease”, international (PCT) patent publication number WO 2014/191894 A1, published 4 Dec. 2014.
Blank et al., 2014b, “Pyrazolopyrrolidine derivatives and their use in the treatment of disease”, international (PCT) patent publication number WO 2014/191896 A1, published 4 Dec. 2014.
Blank et al., 2014c, “Pyrazolo-pyrrolidin-4-one derivatives as BET inhibitors and their use in the treatment of disease”, international (PCT) patent publication number WO 2014/191906 A1, published 4 Dec. 2014.
Blank et al., 2014d, “Pyrazolo-pyrrolidin-4-one derivatives and their use in the treatment of disease”, international (PCT) patent publication number WO 2014/191911 A1, published 4 Dec. 2014.
Blank et al., 2015, “Pyrrolopyrrolone derivatives and their use as BET inhibitors”, international (PCT) patent publication number WO 2015/075665 A1, published 28 May 2015.
Chao et al., 2012, “The CD47-SIRPα pathway in cancer immune evasion and potential therapeutic implications”, Current Opinions in Immunology, Vol. 24, No. 2, pp. 225-232.
Cynis et al., 2013, “Inhibition of Glutaminyl Cyclases alleviates CCL2-mediated inflammation of non-alcoholic fatty liver disease in mice”, International Journal of Experimental Pathology, Vol. 94, No. 3, pp. 217-225.
Dubois et al., 2011, “SIRPA is a specific cell-surface marker for isolating cardiomyocytes derived from human pluripotent stem cells”, Nature Biotechnology, Vol. 29, No. 11, pp. 1011-1018.
Hellvard et al., 2012, “Glutaminyl Cyclases as Novel Targets for the Treatment of Septic Arthritis” The Journal of Infectious Diseases, Vol. 207, No. 5, pp. 768-777.
Huang et al., 2011, “Structures of Human Golgi-resident Glutaminyl Cyclase and Its Complexes with Inhibitors Reveal a Large Loop Movement”, J. Biol. Chem., Vol. 286, pp. 12439-12449.
Holliger et al., 2005, “Engineered antibody fragments and the rise of single domains”, Nature Biotechnology, Vol. 23, No. 9, pp. 1126-1136.
Isenberg et al., 2007, “Increasing Survival of Ischemic Tissue by Targeting CD47”, Circulation Research, Vol. 100, pp. 712-720.
Isenberg et al., 2018, “The role of CD47 in pathogenesis and treatment of renal ischemia reperfusion injury”, Pediatric Nephrology, pp. 1-16.
Jimenez-Sanchez et al., 2015, “siRNA screen identifies QPCT as a druggable target for Huntington's disease”, Nature Chemical Biology, Vol. 11, pp. 347-354.
Jin et al., 2009, “CD47 gene knockout protects against transient focal cerebral ischemia in mice”, Experimental Neurology, Vol. 217, No. 1, pp. 165-170.
Kojima et al., 2016, “CD47-blocking antibodies restore phagocytosis and prevent atherosclerosis”, Nature, Vol. 536, pp. 86-90.
Kotecki et al., 1999, “Isolation and characterization of a near-haploid human cell line”, Exp. Cell Res., Vol. 252, No. 2, pp. 273-280.
Li et al., 1997, “Mammalian cell expression of dimeric small immune proteins (SIP)”, Protein Engineering, Design and Selection, Vol. 10, No. 6, pp. 731-736.
Li et al., April 2019, “CD47 deficiency protects cardiomyocytes against hypoxia/reoxygenation injury by rescuing autophagic clearance”, Molecular Medicine Reports, Vol. 19, No. 6, pp. 5453-5463.
Lin et al., 2014, “CD47 blockade reduces ischemia/reperfusion injury and improves survival in a rat liver transplantation model”, Transplantation, Vol. 98, No. 4, pp. 394-401.
Lindberg et al., 1993, “Molecular cloning of integrin-associated protein: an immunoglobulin family member with multiple membrane-spanning domains implicated in αvβ3-dependent ligand binding”, Journal of Cell Biology, Vol. 123, No. 2, pp. 485-496.
Logtenberg et al., 2019, “Glutaminyl cyclase is an enzymatic modifier of the CD47-SIRPα axis and a target for cancer immunotherapy”, Nature Medicine, Vol. 25, No. 4, pp. 612-619.
Lu et al., 2020, “Development of therapeutic antibodies for the treatment of diseases”, J. Biomedical Science, Vol. 27, Article No. 1.
Majeti et al., 2009, “CD47 Is an Adverse Prognostic Factor and Therapeutic Antibody Target on Human Acute Myeloid Leukemia Stem Cells”, Cell, Vol. 138, No. 2, pp. 286-299.
Matozaki et al., 2009, “Functions and molecular mechanisms of the CD47-SIRPα signalling pathway”, Trends in Cell Biology, Vol. 19, No. 2, pp. 72-80.
Nagaoka et al., 2019, “PfMSA180 is a novel Plasmodium falciparum vaccine antigen that interacts with human erythrocyte integrin associated protein (CD47)”, Scientific Reports, Vol. 9, Article No. 5923.
Novelli et al., June 2019, “Vascular TSP1-CD47 signaling promotes sickle cell-associated arterial vasculopathy and pulmonary hypertension in mice”, Amer. J. Physiology, Lung Cellular and Molecular Physiology, Vol. 316, No. 6, pp. L1150-L1164.
Raymond et al., 2009, “Selective control of SIRP-α-positive airway dendritic cell trafficking through CD47 is critical for the development of T_{H}2-mediated allergic inflammation”, The Journal of Allergy and Clinical Immunology, Vol. 124, No. 6, pp. 1333-1342.
Rogers et al., 2012, “Activation of Parenchymal CD47 Promotes Renal Ischemia-Reperfusion Injury”, Journal of the American Society of Nephrology, Vol. 23, No. 9, pp. 1538-1550.
Rogers et al., 2016, “CD47 regulates renal tubular epithelial cell self-renewal and proliferation following renal ischemia reperfusion”, Kidney International, Vol., 90, No. 2, pp. 334-347.
Rosenbloom et al., 2010, “Narrative review: fibrotic diseases: cellular and molecular mechanisms and novel therapies”, Ann. Intern. Med., Vol. 152, pp. 159-166.
Schilling et al., 2008, “Glutaminyl cyclase inhibition attenuates pyroglutamate Aβ and Alzheimer's disease-like pathology”, Nature Medicine, Vol. 14, pp. 1106-1111.
Uger et al., 2020, “Blockade of the CD47-SIRPα axis: a promising approach for cancer immunotherapy”, Expert Opinion on Biological Therapy, Vol. 20, No. 1, pp. 5-8.
van den Berg et al., 2008, “Innate immune ‘self’ recognition: a role for CD47-SIRPα interactions in hematopoietic stem cell transplantation”, Trends in Immunology, Vol. 29, No 5, pp. 203-206.
Wang et al., 2016, “RNAi-Mediated Down-Regulation of CD47 Protects against Ischemia/Reperfusion-Induced Myocardial Damage via Activation of eNOS in a Rat Model”, Cellular Physiology and Biochemistry, Vol. 40, No. 5, pp. 1163-1174.
Wang et al., 2017, “CD47 blockade reduces ischemia/reperfusion injury in donation after cardiac death rat kidney transplantation”, American Journal of Transplantation, Vol. 18, No. 4, pp. 843-854.
Wernig et al., 2017, “Unifying mechanism for different fibrotic diseases”, PNAS, Vol. 114, No. 18, pp. 4757-4762.
Wiersma et al., 2015, “CD47, a multi-facetted target for cancer immunotherapy”, Atlas of Genetics and Cytogenetics in Oncology and Haematology, Vol. 19, No. 6, pp. 417-431.
Wynn et al., 2004, “Fibrotic disease and the T(H)1/T(H)2 paradigm”, Nat. Rev. Immunol., Vol. 4, No. 8, pp. 585-594.
Xiao et al., 2014, “CD47 blockade reduces ischemia/reperfusion injury and improves survival in a rat liver transplantation model”, Liver Transplanation, Vol. 21, pp. 468-477.
Xiao et al., 2016, “Attenuation of Ischemia-Reperfusion Injury and Improvement of Survival in Recipients of Steatotic Rat Livers Using CD47 Monoclonal Antibody”, Transplantation, Vol. 100, No. 7, pp. 1480-1489.
Xu et al., 2017, “Anti-CD47 monoclonal antibody therapy reduces ischemia-reperfusion injury of renal allografts in a porcine model of donation after cardiac death”, American Journal of Transplantation, Vol. 18, No. 4, pp. 855-867.
Zhang et al., 2017, “Acute CD47 Blockade During Ischemic Myocardial Reperfusion Enhances Phagocytosis-Associated Cardiac Repair”, JACC: Basic to Translational Science, Vol. 2, No. 4, pp. 386-397.
Zhou et al., 2014, “Brain CD47 expression in a swine model of intracerebral hemorrhage”, Brain Research, Vol. 1574, pp. 70-76.

1. A compound selected from compounds of the following formula, and pharmaceutically acceptable salts, hydrates, and solvates thereof: 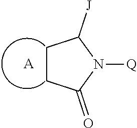
wherein Ring A is a 5-membered heteroaromatic ring having:
exactly 2 ring heteroatoms, wherein each ring heteroatom is N; or
exactly 2 ring heteroatoms, wherein one ring heteroatom is N and the other ring heteroatom is S; or
exactly 2 ring heteroatoms, wherein one ring heteroatom is N and the other ring heteroatom is O; or
exactly 3 ring heteroatoms, wherein each ring heteroatom is N; or
exactly 1 ring heteroatom, wherein the ring heteroatom is N;
and wherein, in Ring A:
a non-bridging ring atom that is N may optionally be substituted with a group —R^{ANN};
a non-bridging ring atom that is C may optionally be substituted with a group —R^{ACC};
wherein —R^{ACC}, or each —R^{ACC} if there are two or more, is independently selected from:
—R^{T},
—R^{TX},
—F, —Cl, —Br, —I,
—OH, —OR^{TT}, —OR^{TX},
L^{TT}-OH, -L^{TT}-OR^{TT}, -L^{TT}-OR^{TX},
—NH₂, —NHR^{TT}, —NR^{TT}₂, —NHR^{TX},
L^{TT}-NH₂, -L^{TT}-NHR^{TT}, -L^{TT}-NR^{TT}₂,
—C(═O)R^{TT},
—C(═O)OH, —C(═O)OR^{TT}, —OC(═O)R^{TT},
—C(═O)NH₂, —C(═O)NHR^{TT}, —C(═O)NR^{TT}₂,
—NHC(═O)R^{TT}, —NR^{TN}C(═O)R^{TT},
—NHC(═O)NH₂, —NHC(═O)NHR^{TT}, —NHC(═O)NR^{T2},
—NR^{TN}C(═O)NH₂, —NR^{TN}C(═O)NHR^{TT}, —NR^{TN}C(═O)NR^{TT}₂,
—NHC(═O)OR^{TT}, —NR^{TN}C(═O)OR^{TT},
—OC(═O)NH₂, —OC(═O)NHR^{TT}, —OC(═O)NR^{TT}₂,
—S(═O)₂NH₂, —S(═O)₂NHR^{TT}, —S(═O)₂NR^{TT}₂,
—NHS(═O)₂R^{T}, —NR^{TN}S(═O)₂R^{TT},
S(═O)(═NH)—NH₂, —S(═O)(═NH)—NHR^{TT}, —S(═O)(═NH)—NR^{TT}₂,
—S(═O)(═NR^{TT})—NH₂, —S(═O)(═NR^{TT})—NHR^{TT}, —S(═O)(═NR^{TT})—NR^{TT}₂,
—N═S(═O)(R^{T})—NH₂, —N═S(═O)(R^{TT})—NHR^{TT}, —N═S(═O)(R^{TT})—NR^{TT}₂,
—NH—S(═O)(═NH)—R^{TT}, —NH—S(═O)(═NR^{TT})—R^{TT},
—NR^{TN}—S(═O)(═NH)—R^{TT}, —NR^{TN}—S(═O)(═NR^{TT})—R^{TT},
—S(═O)R^{TT}, —S(═O)₂R^{TT},
—SH, —SR^{TT}, —SR^{TX},
—CN, and —NO₂;
wherein —R^{ANN}, or each —R^{ANN} if there are two or more, is independently selected from:
—R^{T},
—R^{TX},
L^{TT}-OH, -L^{TT}-OR^{TT}, -L^{TT}-OR^{TX},
L^{TT}-NH₂, -L^{TT}-NHR^{TT}, -L^{TT}-NR^{TT}₂,
—C(═O)R^{TT},
—C(═O)OR^{TT},
—C(═O)NH₂, —C(═O)NHR^{TT}, —C(═O)NR^{TT}₂,
—S(═O)₂NH₂, —S(═O)₂NHR^{TT}, —S(═O)₂NR^{TT}₂,
—S(═O)R^{TT}, and —S(═O)₂R^{TT};
wherein:
each —R^{T} is independently selected from:
—R^{T1}, —R^{T2}, —R^{T3}, —R^{T4}, —R^{T5},
L^{T}-R^{T2}, -L^{T}-R^{T3}, -L^{T}-R^{T4}, and -L^{T}-R^{T5};
each —R^{TT} is independently selected from:
—R^{T1}, —R^{T2}, —R^{T3}, —R^{T4}, —R^{T5},
L^{T}-R^{T2}, -L^{T}-R^{T3}, -L^{T}-R^{T4}, and -L^{T}-R^{T5};
each —R^{TX} is independently linear or branched saturated C₁₋₄fluoroalkyl;
each —R^{TN} is independently linear or branched saturated C₁₋₄alkyl;
each -L^{TT}- is independently linear or branched saturated C₁₋₄alkylene;
wherein:
each —R^{T1} is independently linear or branched saturated C₁₋₆alkyl;
each —R^{T2} is saturated C₃₋₆cycloalkyl;
each —R^{T3} is non-aromatic C₄₋₉heterocyclyl;
each —R^{T4} is independently phenyl or naphthyl;
each —R^{T5} is C5-12heteroaryl;
each -L^{T}- is independently linear or branched saturated C₁₋₄alkylene;
wherein each —R^{T2}, —R^{T3}, R^{T4} and —R^{T5} is optionally substituted with one or more groups independently selected from:
—R^{TTT}, —R^{TTTX},
—F, —Cl, —Br, —I,
—OH, —OR^{TTT}, —OR^{TTTX},
—NH₂, —NHR^{TTT}, —NHR^{TTTX}, —NR^{TTT}₂,
—C(═O)R^{TTT}, —C(═O)OH, and —C(═O)OR^{TTT};
wherein:
each —R^{TTT} is independently selected from linear or branched saturated C₁₋₄alkyl, saturated C₃₋₆cycloalkyl, phenyl, and benzyl;
each —R^{TTTX} is independently linear or branched saturated C₁₋₄fluoroalkyl;
and wherein -Q is independently selected from: 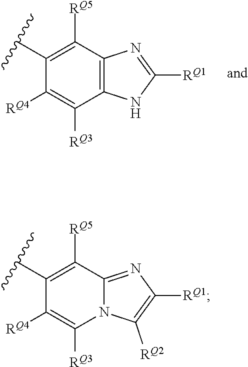
wherein:
each —R^{Q1} is independently —H or —R^{QQ1};
—R^{Q2} is independently —H or —R^{QQ2};
each —R^{Q3} is independently —H or —R^{QQ3};
each —R^{Q4} is independently —H or —R^{QQ4};
each —R^{Q5} is independently —H or —R^{QQ5}; and
each —R^{QQ1}, —R^{QQ2}, —R^{QQ3} —R^{QQ4}, and —R^{QQ5} is independently —R^{Q};
wherein each —R^{Q} is independently selected from:
—R^{QQ},
—R^{QX},
—F, —Cl, —Br, —I,
—OH, —OR^{QQ}, —OR^{QX},
—NH₂, —NHR^{QQ}, —NHR^{QX}, —NR^{QQ}₂, and
—CN;
wherein:
each —R^{QQ} is independently —R^{QQQ1} or —R^{QQQ2};
each —R^{QQQ1} is independently linear or branched saturated C₁₋₄alkyl;
each —R^{QQ2} is saturated C₃₋₆cycloalkyl;
each —R^{QX} is independently linear or branched saturated C₁₋₄fluoroalkyl;
and wherein -J is the following group: 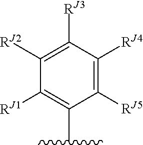
wherein:
—R^{J1} is independently —H or —R^{JJ1};
—R^{J2} is independently —H or —R^{JJ2};
—R^{J3} is independently —H or —R^{JJ3};
—R^{J4} is independently —H or —R^{JJ4}; and
—R^{J5} is independently —H or —R^{JJ5};
wherein:
each of —R^{JJ1}, —R^{JJ2} —R^{JJ3} —R^{JJ4}, and —R^{JJ5} is independently —R^{J};
wherein each —R^{J} is independently selected from:
—R^{P},
—R^{PX},
—F, —Cl, —Br, —I,
—OH, —OR^{PP}, —OR^{PX},
L^{PP}-OH, -L^{PP}-OR^{PP}, -L^{PP}-OR^{PX},
—NH₂, —NHR^{PP}, —NR^{PP}₂, —NHR^{PX},
L^{PP}-NH₂, -L^{PP}-NHR^{PP}, -L^{PP}-NR^{PP}₂,
—C(═O)R^{PP},
—C(═O)OH, —C(═O)OR^{PP}, —OC(═O)R^{PP},
—C(═O)NH₂, —C(═O)NHR^{PP}, —C(═O)NR^{PP}₂,
—NHC(═O)R^{PP}, —NR^{PN}C(═O)R^{PP},
—NHC(═O)NH₂, —NHC(═O)NHR^{PP}, —NHC(═O)NR^{PP}₂,
—NR^{PN}C(═O)NH₂, —NR^{PN}C(═O)NHR^{PP}, —NR^{PN}C(═O)NR^{PP}₂,
—NHC(═O)OR^{PP}, —NR^{PN}C(═O)OR^{PP},
—OC(═O)NH₂, —OC(═O)NHR^{PP}, —OC(═O)NR^{PP}₂,
—S(═O)₂NH₂, —S(═O)₂NHR^{PP}, —S(═O)₂NR^{PP}₂,
—NHS(═O)₂R^{PP}, —NR^{PN}S(═O)₂R^{PP},
—S(═O)(═NH)—NH₂, —S(═O)(═NH)—NHR^{PP}, —S(═O)(═NH)—NR^{PP}₂,
—S(═O)(═NR^{PP})—NH₂, —S(═O)(═NR^{PP})—NHR^{PP}, —S(═O)(═NR^{PP})—NR^{PP}₂,
—N═S(═O)(R^{PP})—NH₂, —N═S(═O)(R^{PP})—NHR^{PP}, —N═S(═O)(R^{PP})—NR^{PP}₂,
—NH—S(═O)(═NH)—R^{PP}, —NH—S(═O)(═NR^{P}P)—R^{PP},
—NR^{PN}—S(═O)(═NH)—R^{PP}, —NR^{PN}—S(═O)(═NR^{PP})—REP,
—S(═O)R^{PP}, —S(═O)₂R^{PP},
—SH, —SR^{PP}, —SR^{PX},
—CN, and —NO₂;
wherein:
each —R^{P} is independently selected from:
—R^{P1}, —R^{P2}, —R^{P3}, —R^{P4}, —R^{P5},
L^{P}-R^{P2}, -L^{P}-R^{P3}, -L^{P}-R^{P4}, and -L^{P}-R^{P5};
each —R^{PP} is independently selected from:
—R^{P1}, —R^{P2}, —R^{P3}, —R^{P4}, —R^{P5},
L^{P}-R^{P2}, -L^{P}-R^{P3}, -L^{P}-R^{P4}, and -L^{P}-R^{P5};
each —R^{PX} is independently linear or branched saturated C₁₋₄fluoroalkyl;
each —R^{PN} is independently linear or branched saturated C₁₋₄alkyl;
each -L^{PP}- is independently linear or branched saturated C₁₋₄alkylene;
wherein:
each —R^{P1} is independently linear or branched saturated C₁₋₆alkyl;
each —R^{P2} is saturated C₃₋₆cycloalkyl;
each —R^{P3} is non-aromatic C₄₋₉heterocyclyl;
each —R^{P4} is independently phenyl or naphthyl;
each —R^{P5} is C₅₋₁₂heteroaryl;
each -L^{P}- is independently linear or branched saturated C₁₋₄alkylene;
wherein each —R^{P2}, —R^{P3}, —R^{P4}, and —R^{P5} is optionally substituted with one or more groups independently selected from:
—R^{PPP}, —R^{PPPX},
—F, —Cl, —Br, —I,
—OH, —OR^{PPP}, —OR^{PPPX},
—NH₂, —NHR^{PP}, —NHR^{PPPX}, —NR^{PPP}₂,
—C(═O)R^{PPP}, —C(═O)OH, and —C(═O)OR^{PPP},
—S(═O)₂R^{PPP}; and
—CN;
and wherein, additionally, each —R^{P2} and —R^{P3} is optionally substituted with ═O;
wherein:
each —R^{PPP} is independently selected from linear or branched saturated C₁₋₄alkyl, saturated C₃₋₆cycloalkyl, phenyl, and benzyl;
each —R^{PPPX} is independently linear or branched saturated C₁₋₄fluoroalkyl;
and additionally:
—R^{JJ1} and —R^{JJ2}, if present, taken together with the atoms to which they are attached, may form a fused 5- or 6-membered ring (i.e., fused to the phenyl ring to which they are attached); or
—R^{JJ2} and —R^{JJ3}, if present, taken together with the atoms to which they are attached, may form a fused 5- or 6-membered ring (i.e., fused to the phenyl ring to which they are attached),
preferably wherein the ring atom to which -J is attached, marked with an asterisk (*) in the following formula, is in the following configuration:

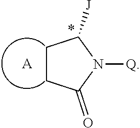

2. The compound according to claim 1, which is a compound of one of the following formulae, or a pharmaceutically acceptable salt, hydrate, or solvate thereof: 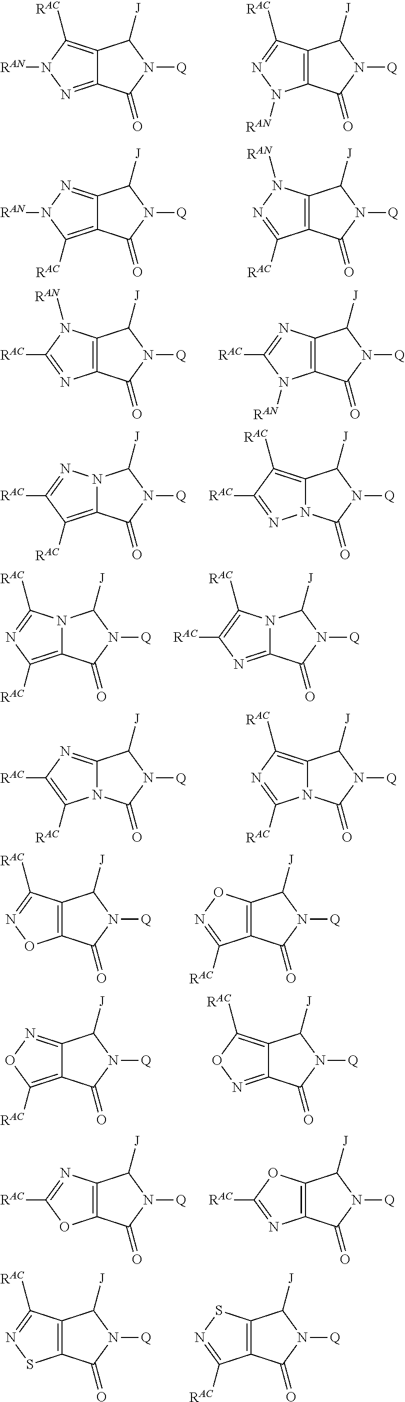 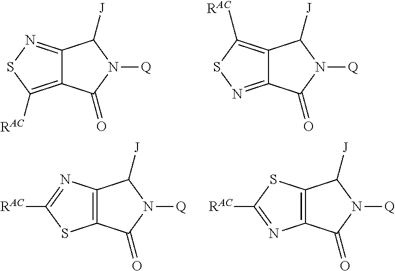
wherein:
each —R^{AC} is independently —H or —R^{ACC}; and
each —R^{AN} is independently —H or —R^{ANN}.

3-9. (canceled)

10. The compound according to claim 1, wherein:
—R^{AC}, if present, or each —R^{AC} if there are two or more, is H; and/or
—R^{AN}, if present, or each —R^{AN} if there are two or more, is H; and/or
—R^{ACC}, if present, or each —R^{ACC} if there are two or more, is independently selected from: —R^{T} and —R^{TX}; or —R^{ACC}, if present, or each —R^{ACC} if there are two or more, is —R^{T}; and/or
—R^{ANN}, if present, or each —R^{ANN} if there are two or more, is independently selected from: —R^{T}, —R^{TX}, -L^{TT}-OR^{TT}, and -L^{TT}-OR^{TX}; or —R^{ANN}, if present, or each —R^{ANN} if there are two or more, is —R^{T}; and/or
each —R^{T}, if present, is independently selected from: —R^{T1}, —R^{T2}, and -L^{T}-R^{T2}; or each —R^{T}, if present, is —R^{T1}; and/or
each —R^{TT}, if present, is —R^{T1}; and/or
each —R^{TX}, if present, is independently selected
from: —CF₃, —CHF₂, —CH₂CF₃, —CH₂CH₂F, —CH₂CHF₂, —CH(CH₃)CF₃, and —CH₂C(CH₃)₂F; and/or
each —R^{T1}, if present, is independently linear or branched saturated C₁₋₃alkyl; and/or
each —R^{T2}, if present, is independently selected from: cyclopropyl and cyclobutyl; and/or
each —R^{T3}, if present, is non-aromatic monocyclic C₄₋₇heterocyclyl; and/or
—R^{T4}, if present, is phenyl.

11-23. (canceled)

24. The compound according to claim 1, wherein -Q is one of the following formulae: 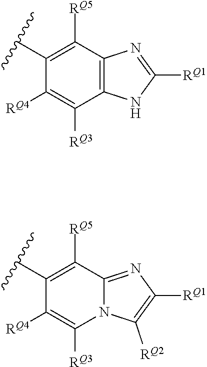
wherein:
—R^{Q1} is independently —H or —R^{QQ1};
—R^{Q2} is independently —H or —R^{QQ2};
—R^{Q3} is independently —H or —R^{QQ3};
—R^{Q4} is independently —H or —R^{QQ4};
—R^{Q5} is independently —H or —R^{QQ5}; and
each of —R^{QQ1}, —R^{QQ2}, —R^{QQ3}, —R^{QQ4}, and —R^{QQ5} is independently —R^{Q},
preferably wherein -Q is one of the following formulae:

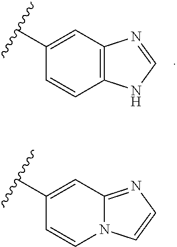 [SMILES: *c1ccc2[nH]cnc2c1 ; *c1ccn2ccnc2c1]

25-28. (canceled)

29. The compound according to claim 1, wherein:
—R^{J4} is —H; and
—R^{J5} is —H.

30. The compound according to claim 1, wherein -J is independently selected from the following groups:

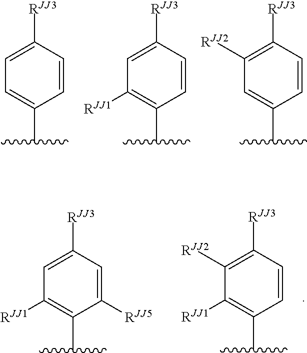

31-33. (canceled)

34. The compound according to claim 1, wherein —R^{JJ1}, if present, is independently selected from:
—R^{P},
—R^{PX},
—F, —Cl, —Br,
—OH, —OR^{PP}, and —OR^{PX}.

35. (canceled)

36. The compound according to claim 1, wherein —R^{JJ2}, if present, is independently —F.

37. The compound according to claim 1, wherein —R^{JJ3}, if present, is independently selected from —R^{P}, —OR^{PP}, —OR^{PX}, —NHR^{PP}, and —NR^{PP}₂.

38-39. (canceled)

40. The compound according to claim 1, wherein each —R^{P}, if present, is independently selected from:
—R^{P1}, —R^{P2}, —R^{P3}, —R^{P4}, and —R^{P5}.

41-43. (canceled)

44. The compound according to claim 1, wherein each —R^{PP}, if present, is —R^{P1}.

45. The compound according to claim 1, wherein each —R^{PX}, if present, is independently selected from: —CF₃, —CHF₂, —CH₂CF₃, —CH₂CH₂F, —CH₂CHF₂, —CH(CH₃)CF₃, —CH₂C(CH₃)₂F, —CH₂CF₂CH₃, —CH₂CH₂CF₂CH₃, —CH₂CH₂CHF₂, and —CH₂CH₂CF₃.

46. The compound according to claim 1, wherein each —R^{P1}, if present, is -Me.

47. The compound according to claim 1, wherein each —R^{P2}, if present, is independently selected from: cyclopropyl and cyclobutyl.

48. The compound according to claim 1, wherein:
each —R^{P3}, if present, is independently selected from: oxetanyl; tetrahydrofuranyl; tetrahydropyranyl; oxanyl; dioxanyl; azetidinyl; pyrrolidinyl; piperidinyl; piperazinyl; morpholinyl; thiomorpholinyl, 1,4-thiazinane 1,1-dioxide; azepanyl; oxazepanyl; diazepanyl; 2,5-diazabicyclo[2.2.1]heptane; 6-oxa-3-azabicyclo[3.1.1]heptane; 2-oxa-5-azabicyclo[2.2.1]heptane; 5-oxa-2-azabicyclo[4.1.0]heptane; 8-oxa-3-azabicyclo[3.2.1]octane; 3-oxa-8-azabicyclo[3.2.1]octane; 4-oxa-7-azabicyclo[3.2.0]heptane; 3,3a,4,5,6,6a-hexahydro-1H-furo[3,4-c]pyrrole; 6-oxa-3-azaspiro[3.3]heptane; 8-oxa-2-azaspiro[3.4]octane; 7-oxa-2-azaspiro[3.4]octane; 2-oxa-7-azaspiro[3.4]octane; and 8-oxa-3-azaspiro[4.4]nonane; and/or
each —R^{P3}, if present, is non-aromatic monocyclic C₄₋₇heterocyclyl; and/or
each —R^{P3}, if present, is independently selected from: non aromatic bridged C₇₋₉heterocyclyl and non aromatic spiro C₇₋₉heterocyclyl.

49-51. (canceled)

52. The compound according to claim 1, wherein each —R^{P4}, if present, is phenyl.

53. The compound according to claim 1, wherein each —R^{P5}, if present, is C₅₋₆heteroaryl, preferably independently selected from: imidazolyl; oxazolyl; isoxazolyl; thiazolyl; isothiazolyl; and pyrazolyl.

54. (canceled)

55. The compound according to claim 1, wherein:
—R^{J3} is —R^{JJ3}; and —R^{JJ3} is —R^{P}; and that —R^{P} is —R^{P3}; or
—R^{J3} is —R^{JJ3}; and —R^{JJ3} is —R^{P}; and that —R^{P} is —R^{P3}; and:
—R^{P3} is independently selected from: azetidino, pyrrolidino, piperidino, piperazino, morpholino, thiomorpholino, azepano, and diazepano: or
—R^{P3} is independently selected from N-linked: 2,5-diazabicyclo[2.2.1]heptane; 6-oxa-3-azabicyclo[3.1.1]heptane; 2-oxa-5-azabicyclo[2.2.1]heptane; 5-oxa-2-azabicyclo[4.1.0]heptane; 8-oxa-3-azabicyclo[3.2.1]octane; 3-oxa-8-azabicyclo[3.2.1]octane; 4-oxa-7-azabicyclo[3.2.0]heptane; 3,3a,4,5,6,6a-hexahydro-1H-furo[3,4-c]pyrrole; 6-oxa-3-azaspiro[3.3]heptane; 8-oxa-2-azaspiro[3.4]octane; 7-oxa-2-azaspiro[3.4]octane; 2-oxa-7-azaspiro[3.4]octane; and 8-oxa-3-azaspiro[4.4]nonane; or
—R^{J3} is —R^{JJ3}; and —R^{JJ3} is —R^{P}; and that —R^{P} is —R^{P5}; and —R^{P5} is C₅heteroaryl: or
—R^{J3} is —R^{JJ3}; and —R^{JJ3} is —R^{P}; and that —R^{P} is —R^{P5}; and —R^{P5} is independently selected from: imidazolyl; oxazolyl; isoxazolyl; thiazolyl; isothiazolyl; and pyrazolyl; or
—R^{J3} is —R^{JJ3}; and —R^{JJ3} is —R^{P}; and that —R^{P} is —R^{P5}; and —R^{P5} is independently selected from the following (and is optionally substituted with one or more groups as described herein): 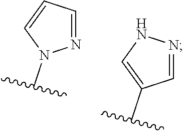 [SMILES: *n1cccn1 ; *c1cn[nH]c1]
or
—R^{J3} is —R^{JJ3}; and —R^{JJ3} is —R^{P}; and that —R^{P} is —R^{P5}; and —R^{P5} is independently selected from the following: 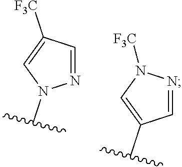 [SMILES: *n1cc(C(F)(F)F)cn1 ; *c1cnn(C(F)(F)F)c1]
or
—R^{J3} is —R^{JJ3}; and —R^{JJ3} is —OR^{PP}; and that —R^{PP} is —R^{P1}; or
—R^{J3} is —R^{JJ3}; and —R^{JJ3} is —OR^{PX}.

56-62. (canceled)

63. The compound according to claim 1, which is a compound of one of the following formulae, or a pharmaceutically acceptable salt, hydrate, or solvate thereof:
FRPPO-001, FRPPO-002, FRPPO-003, FRPPO-004, FRPPO-005, FRPPO-006, FRPPO-007, FRPPO-008, FRPPO-009, FRPPO-010, FRPPO-011, FRPPO-012, FRPPO-013, FRPPO-014, FRPPO-015, FRPPO-016, FRPPO-017, FRPPO-018, FRPPO-019, FRPPO-020, FRPPO-021, FRPPO-022, FRPPO-023, FRPPO-024, FRPPO-025, FRPPO-026, FRPPO-027, FRPPO-028, FRPPO-029, FRPPO-030, FRPPO-031, FRPPO-032, FRPPO-033, FRPPO-034, FRPPO-035, FRPPO-036, FRPPO-037, FRPPO-038, FRPPO-039, FRPPO-046, FRPPO-047, FRPPO-048, FRPPO-049, FRPPO-050, FRPPO-051, FRPPO-052, FRPPO-053, FRPPO-054, FRPPO-057, FRPPO-058, FRPPO-061, FRPPO-063, FRPPO-064, FRPPO-066, FRPPO-067, FRPPO-068, FRPPO-069, FRPPO-070, FRPPO-072, FRPPO-073, FRPPO-074, FRPPO-076, FRPPO-077, FRPPO-078, FRPPO-079, FRPPO-081, FRPPO-082, FRPPO-083, FRPPO-084, FRPPO-085, FRPPO-086, FRPPO-087, FRPPO-088, FRPPO-089, FRPPO-090, FRPPO-091, FRPPO-092, FRPPO-093, FRPPO-094, FRPPO-095, FRPPO-096, FRPPO-097, FRPPO-098, FRPPO-100, FRPPO-101, FRPPO-102, FRPPO-103, FRPPO-104, FRPPO-105, FRPPO-106, FRPPO-107, FRPPO-108, FRPPO-109, FRPPO-110, FRPPO-111, FRPPO-112, FRPPO-113, FRPPO-114, FRPPO-115, FRPPO-126, FRPPO-127, FRPPO-134, FRPPO-135, FRPPO-136, FRPPO-137, FRPPO-142, FRPPO-143, FRPPO-144, FRPPO-145, FRPPO-146, FRPPO-147, FRPPO-148, FRPPO-149, FRPPO-150, FRPPO-151, FRPPO-152, FRPPO-153, FRPPO-154, FRPPO-155, FRPPO-156, FRPPO-157, FRPPO-158, FRPPO-159, FRPPO-160, FRPPO-161, FRPPO-162, FRPPO-164, FRPPO-165, FRPPO-166, FRPPO-167, FRPPO-168, FRPPO-169, FRPPO-170, FRPPO-171, FRPPO-173, FRPPO-174, FRPPO-175, FRPPO-176, FRPPO-177, FRPPO-197, and FRPPO-198.

64. (canceled)

65. A pharmaceutical composition comprising the compound according to claim 1, and a pharmaceutically acceptable carrier or diluent.

66. (canceled)

67. A method of inhibiting glutaminyl-peptide cyclotransferase-like (isoQC) enzyme and/or glutaminyl-peptide cyclotransferase (QC) enzyme, or such an enzyme in a cell, in vitro or in vivo, comprising contacting the isoQC and/or QC enzyme, or the cell, with an effective amount of the compound according to claim 1.

68-71. (canceled)

72. A method of treatment of a disorder of the human or animal body that is ameliorated by the inhibition of glutaminyl-peptide cyclotransferase-like (isoQC) enzyme and/or glutaminyl-peptide cyclotransferase (QC) enzyme, comprising administering to a subject in need of treatment a therapeutically-effective amount of the compound according to claim 1.

73-74. (canceled)

75. A method of treatment of a disorder, comprising administering to a subject in need of treatment a therapeutically-effective amount of the compound according to claim 1, wherein the disorder is selected from:
a proliferative disorder;
cancer;
leukemia, acute myeloid leukemia (AML), acute promyelocytic leukemia (APL), chronic myeloid leukemia (CML), acute lymphoblastic leukemia (ALL), T-cell acute lymphoblastic leukemia (T-ALL), lymphoma, B-cell lymphoma, T-cell lymphoma, Hodgkin's disease, non-Hodgkin's lymphoma (NHL), hairy cell lymphoma, Burkett's lymphoma, multiple myeloma (MM), myelodysplastic syndrome, lung cancer, adenocarcinoma, small cell lung cancer (SCLC), non-small cell lung cancer (NSCLC), mediastinum cancer, peritoneal cancer, mesothelioma, gastrointestinal cancer, gastric cancer, stomach cancer, bowel cancer, small bowel cancer, large bowel cancer, colon cancer, colon adenocarcinoma, colon adenoma, rectal cancer, colorectal cancer, leiomyosarcoma, breast cancer, gynaecological cancer, genito-urinary cancer, ovarian cancer, endometrial cancer, cervical cancer, prostate cancer, testicular cancer, seminoma, teratocarcinoma, liver cancer, kidney cancer, bladder cancer, urothelial cancer, biliary tract cancer, pancreatic cancer, exocrine pancreatic carcinoma, esophageal cancer, nasopharyngeal cancer, head and neck squamous cell carcinoma (HNSCC), skin cancer, squamous cancer, squamous cell carcinoma, Kaposi's sarcoma, melanoma, malignant melanoma, xeroderma pigmentosum, keratoacanthoma, bone cancer, bone sarcoma, osteosarcoma, rhabdomyosarcoma, fibrosarcoma, thyroid gland cancer, thyroid follicular cancer, adrenal gland cancer, nervous system cancer, brain cancer, astrocytoma, neuroblastoma, glioma, schwannoma, glioblastoma, or sarcoma;
atherosclerosis;
a fibrotic disease;
scleroderma, idiopathic pulmonary fibrosis, liver cirrhosis, kidney fibrosis, lung fibrosis, bladder fibrosis, heart fibrosis, pancreas fibrosis, or myelofibrosis;
an infectious disease;
an infectious disease caused by a virus, bacterium, or protozoan;
an infectious disease caused by a pathogen selected from: a lentivirus, human T-lymphotropic virus (HTLV), an hepadna virus, hepatitis B virus, a herpes virus, human papilloma virus, la crosse virus, Yersinia sp., Yersinia pestis, Yersinia pseudotuberculosis, Yersinia enterocolitica, Franciscella sp., Helicobacter sp., Helicobacter pylori, Pasteurella sp., Vibrio sp., Vibrio cholerae, Vibrio parahemolyticus, Legionella sp., Legionella pneumophila, Listeria sp., Listeria monocytogenes, Mycoplasma sp., Mycoplasma hominis, Mycoplasma pneumoniae, Mycobacterium sp., Mycobacterium tuberculosis, Mycobacterium leprae, Rickettsia sp., Rickettsia rickettsii, Rickettsia typhi, a Plasmodium, a Trypanosoma, a Giardia, a Toxoplasma, and a Leishmania;
Alzheimer's disease;
non-alcoholic steatohepatitis (NASH);
septic arthritis;
chronic obstructive pulmonary disease (COPD);
asthma;
an allergy;
a parasitic infection;
malaria;
sickle-cell anemia;
Huntington's disease;
ischemia;
reperfusion injury;
renal ischemia or reperfusion injury; myocardial ischemia or reperfusion injury;
liver ischemia or reperfusion injury; or cerebral ischemia or reperfusion injury.

76. (canceled)


SEQUENCE LISTING (3 sequences)

SEQ ID NO: 1 - DNA, 427 bp, Artificial Sequence
  synthetic DNA fragment
cagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacggattaattaaaccgtctcagtttaagagctagaaatagcaagtttaaataaggctagtccgttatcaacttgaaaaagtggcaccgagtcggtgcttttttgaattcgctagctaggtcttgaaaggagtgg

SEQ ID NO: 2 - DNA, 75 bp, Artificial Sequence
  Oligonucleotide encoding gRNAs targeting QPCTL
tatcttgtggaaaggacgaaacaccgcggggaggcttccgatcaatgtttaagagctagaaatagcaagtttaaa

SEQ ID NO: 3 - DNA, 75 bp, Artificial Sequence
  Oligonucleotide encoding gRNAs targeting CD47
tatcttgtggaaaggacgaaacaccgctactgaagtatacgtaaaggtttaagagctagaaatagcaagtttaaa